(19) United States
(12) Patent Application Publication
(10) Pub. No.: US 2022/0160854 A1
(43) Pub. Date: May 26, 2022

(54) COMPOSITION AND PROCESS FOR PREPARING VACCINE

(71) Applicant: Treos Bio Limited, London (GB)

(72) Inventors: Levente Molnár, Felsopakony (HU); Eniko R. Toke, Felsopakony (HU); Julianna Lisziewicz, Balatonalmádi (HU); József Tóth, Gyor (HU); Orsolya Lorincz, Budapest (HU); Zsolt Csiszovszki, Budapest (HU); Eszter Somogyi, Balatonalmádi (HU); Katalin Pántya, Budapest (HU); Mónika Megyesi, Mezokeresztes (HU); Péter Páles, Budapest (HU)

(21) Appl. No.: 17/650,360

(22) Filed: Feb. 8, 2022

Related U.S. Application Data
(63) Continuation of application No. 17/249,362, filed on Feb. 26, 2021.
Application No. 17/249,362 is a continuation of application No. 16/559,430, filed on Sep. 3, 2019, now abandoned.

(30) Foreign Application Priority Data
Sep. 4, 2018 (GB) 1814362.8

(51) Int. Cl. A61K 39/00; C07K 16/28; C07K 14/74; A61K 39/39; C12Q 1/6881; A61P 37/04; A61P 35/00

(52) U.S. Cl. CPC A61K 39/001102; C07K 16/2827; C07K 14/70539; A61K 39/39; C12Q 1/6881; C07K 16/2818; A61K 2039/55577; A61P 35/00; A61K 39/0011; A61K 2039/55505; A61K 2039/55566; A61K 2039/55572; A61P 37/04

(57) ABSTRACT

The disclosure relates to polypeptides, polynucleic acids and pharmaceutical compositions comprising polypeptides that find use in the prevention or treatment of cancer. The disclosure also relates to methods of inducing a cytotoxic T cell response in a subject or treating cancer by administering pharmaceutical compositions comprising the peptides, and companion diagnostic methods. The disclosure also relates to a method of preparing a peptide or polynucleic acid for use in a method of inducing a T cell response against a target polypeptide, wherein the method comprises identifying epitopes in the antigen that bind to multiple alleles of receptors of the highest proportion of subjects in a target population.

### CROSS-REFERENCE

This application is a continuation of U.S. application Ser. No. 17/249,362, filed Feb. 26, 2021, which is a continuation of U.S. application Ser. No. 16/559,430, filed on Sep. 3, 2019, which claims priority to UK Application No. 1814362.8, filed on Sep. 4, 2018, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 28, 2021, is named TBL 006C2 SL.txt and is 1,449,565 bytes in size.

### FIELD

The disclosure relates to peptides and compositions that find use in vaccines and immunotherapy, to nucleic acids and vectors that encode such peptides, to methods of designing and producing such peptides, to methods of predicting whether an individual subject will respond to treatment with such peptides, to subject-specific compositions comprising such peptides, and to methods of treatment using such peptides.

### BACKGROUND

For decades, scientists have assumed that chronic diseases were beyond the reach of a person's natural defences. Recently, however, significant tumor regressions observed in individuals treated with antibodies that block immune inhibitory molecules have accelerated the field of cancer immunotherapy. These clinical findings demonstrate that re-activation of existing T cell responses results in meaningful clinical benefit for individuals. These advances have renewed enthusiasm for developing cancer vaccines that induce tumor specific T cell responses.

Despite the promise, current immunotherapy is effective only in a fraction of individuals. In addition, most cancer vaccine trials have failed to demonstrate statistically significant efficacy because of a low rate of tumor regression and antitumor T cell responses in individuals. Similar failures were reported with therapeutic and preventive vaccines that sought to include T cell responses in the fields of HIV and allergy. There is a need to overcome the clinical failures of immunotherapies and vaccines.

### SUMMARY

In antigen presenting cells (APC) protein antigens are processed into peptides. These peptides bind to HLA molecules and are presented on the cell surface as peptide-HLA complexes to T cells. Different individuals express different HLA molecules, and different HLA molecules present different peptides. The inventors have demonstrated that an epitope that binds to a single HLA class I allele expressed in a subject is essential, but not sufficient to induce tumor specific T cell responses. Instead tumour specific T cell responses are optimally activated when an epitope is recognised and presented by the HLA molecules encoded by at least three HLA class I genes of an individual (PCT/EP2018/055231, PCT/EP2018/055232, PCT/EP2018/055230, EP 3370065 and EP 3369431).

Based on this discovery the inventors have developed a method for designing and preparing peptides to induce T cell responses in the highest proportion of subjects in a given target human population and have used this method to design a set of peptides for use in treating cancer.

Accordingly, in a first aspect the disclosure provides a peptide of up to 50 amino acids in length and comprising the amino acid sequence of any of SEQ ID NOs: 1 to 2786 and/or 5432-5931.

In a further aspect, the disclosure provides a polynucleic acid or a vector that encodes a peptide of up to 50 amino acids in length and comprising the amino acid sequence of any of SEQ ID NOs: 1 to 2786 and/or 5432-5931.

In a further aspect, the disclosure provides a panel of two or more of the peptides or two or more of the polynucleic acids or vectors, wherein each peptide comprises, or each polynucleic acid or vector encodes a peptide that comprises, a different amino acid sequence selected from SEQ ID NOs: 1 to 2786 and/or 5432-5931.

In a further aspect, the disclosure provides a pharmaceutical composition or kit, comprising one or more of the peptides, polynucleic acids, vectors or panels, wherein the composition or kit optionally comprise at least one pharmaceutically acceptable diluent, carrier, or preservative.

In a further aspect, the disclosure provides a method of predicting that a specific human subject will have a cytotoxic T cell response and/or a helper T cell response to administration of the pharmaceutical composition or the peptides, polynucleic acids or vectors of the kit, the method comprising
(i) a. determining that the one or more peptides, or encoded peptides, of the pharmaceutical composition or kit, comprise at least one amino acid sequence that is a T cell epitope capable of binding to at least three HLA class I molecules of the subject; and
   b. predicting that the subject will have a cytotoxic T cell response to administration of the pharmaceutical composition; or
(ii) a. determining that the one or more peptides, or encoded peptides, of the pharmaceutical composition or kit comprise at least one amino acid sequence that is a T cell epitope capable of binding to at least three HLA class II molecules of the subject; and
   b. predicting that the subject will have a helper T cell response to administration of the pharmaceutical composition.

In a further aspect, the disclosure provides a method of vaccination, providing immunotherapy or inducing a cytotoxic T cell response in a subject, the method comprising administering to the subject the pharmaceutical composition or the peptides, polynucleic acids or vectors of the kit.

In further aspects, the disclosure provides
the pharmaceutical composition or the peptides, polynucleic acids or vectors of the kit described above for use in a method of vaccination, providing immunotherapy or inducing a cytotoxic T cell response in a subject; and
use of the peptides or polynucleic acids as described above in the manufacture of a medicament for vaccination, providing immunotherapy or inducing a cytotoxic T cell response in a subject.

In a further aspect, the disclosure provides a method of preparing a pharmaceutical composition or kit for use in a method of treating cancer is a specific human subject, the method comprising
a. selecting two or more peptides, or one or more polynucleic acids or vectors according that encode at least two peptides, wherein each peptide, or encoded peptide, comprises an amino acid sequence selected from SEQ ID NOs: 1 to 2786 and/or 5432-5931 that comprises a T cell epitope capable of binding to at least three HLA class I alleles and/or a T cell epitope capable of binding to at least three HLA class II alleles of the specific human subject; and
b. preparing a pharmaceutical composition or kit comprising the two or more peptides, or one or more polynucleic acids or vectors selected in step a.

In a further aspect, the disclosure provides a method of designing, or preparing a peptide, or a polynucleic acid or vector that encodes a peptide, or a panel of peptides, or one or more polynucleic acid or vectors that encode a panel of peptides, for use in a method of inducing a T cell response against a target polypeptide, the method comprising
(i) selecting or defining a model human population comprising a plurality of subjects each defined by HLA class I genotype and/or by HLA class II genotype;
(ii) identifying for each subject of the model population:
   (a) amino acid sequences of the target polypeptide that are a T cell epitope capable of binding to at least three HLA class I molecules of the subject;
   (b) amino acid sequences of the target polypeptide that are a T cell epitope capable of binding to at least three HLA class II molecules of the subject;
   (c) amino acid sequences of the target polypeptide that comprise a T cell epitope capable of binding to at least three HLA class I molecules of the subject and a T cell epitope capable of binding to at least three HLA class II molecules of the subject; or
   (d) amino acid sequences of the target polypeptide that both
      i. are a T cell epitope capable of binding to at least three HLA class II molecules; and
      ii. comprise an amino acid sequence that is a T cell epitope capable of binding to at least three HLA class I molecules of the subject;
(iii) selecting a polypeptide fragment window length of between 9 and 50 amino acids;
(iv) identifying a fragment of the target polypeptide that
   (a) has the length selected in step (iii); and
   (b) comprises an amino acid sequence identified in any one of step (ii) (a) to (d) in the highest proportion of subjects in the model population;
(v) optionally testing the fragment identified in step (iv) against additional pre-defined criteria, rejecting the fragment if the further pre-defined criteria are not met, and repeating step (iv) to identify an alternative fragment of the target polypeptide that
   (a) has the length selected in step (iii); and
   (b) comprises an amino acid sequence identified in step (iv) in the next highest proportion of subjects in the model population;
(vi) optionally repeating step (iv) and further optionally step (v) in one or more further rounds, wherein a further fragment of the target polypeptide is identified in each round, and wherein in each round subjects are excluded from the model population if any of the fragments selected in step (iv) and not rejected in step (v) of any of the preceding rounds comprises an amino acid sequence identified in step (ii) for that subject; and
(vii) designing or preparing a peptide, a polynucleic acid or vector that encodes a peptide, a panel of peptides, or one or more polynucleic acids or vectors that encode a panel of peptides, wherein each peptide comprises one or more of the target polypeptide fragments identified in step (iv), (v) or (vi), optionally wherein the polypeptide fragment is flanked at the N and/or C terminus by additional amino acids that are not part of the sequence of the target polypeptide antigen.

In a further aspect, the disclosure provides a panel peptides, polynucleic acids or vectors designed and/or prepared according to the method, or comprising or encoding two or more peptides designed and/or prepared according to the method.

In a further aspect, the disclosure provides a panel of peptides, or one or more polynucleic acids or vectors encoding a panel of peptides, for use in a method of inducing a T cell response against one or more target polypeptides in a subject of a target human population, wherein each of the peptides, or encoded peptides, comprises an amino acid sequence that is
(a) 9 to 50 amino acids in length; and
(b) comprises a fragment of the one or more target polypeptides, wherein the fragment comprises, in at least 10% of subjects of the intent-to-treat human population:
   a. an amino acid sequence of the target polypeptide that is a T cell epitope capable of binding to at least three HLA class I molecules of the subject;
   b. an amino acid sequence of the target polypeptide that is a T cell epitope capable of binding to at least three HLA class II molecules of the subject;
   c. an amino acid sequence of the target polypeptide that comprise a T cell epitope capable of binding to at least three HLA class I molecules of the subject and a T cell epitope capable of binding to at least three HLA class II molecules of the subject; or
   d. an amino acid sequence of the target polypeptide that both
      i. is a T cell epitope capable of binding to at least three HLA class II molecules; and
      ii. comprise an amino acid sequence that is a T cell epitope capable of binding to at least three HLA class I molecules of the subject.

In a further aspect, the disclosure provides a pharmaceutical composition or kit comprising the panel of peptides, or one or more polynucleic acids or vectors encoding the panel of peptides, wherein the composition or kit optionally comprises at least one pharmaceutically acceptable diluent, carrier, or preservative.

In a further aspect, the disclosure provides a method of vaccination, providing immunotherapy or inducing a cytotoxic T cell response in a subject, the method comprising administering to the subject a pharmaceutical composition or the panel of peptides, polynucleic acids or vectors of the kit.

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended claims, the singular forms “a”, “an”, and “the” include plural referents unless the content clearly dictates otherwise. Thus, for example, reference to “a peptide” includes two or more such peptides.

Section headings are used herein for convenience only and are not to be construed as limiting in any way.

### DESCRIPTION OF THE FIGURES

FIG. 1

ROC curve of HLA restricted PEPI biomarkers.

FIG. 2

ROC curve of ≥1 PEPI3+ Test for the determination of the diagnostic accuracy. AUC=0.73 classifies a fair diagnostic value for the PEPI biomarker.

FIGS. 3A-B

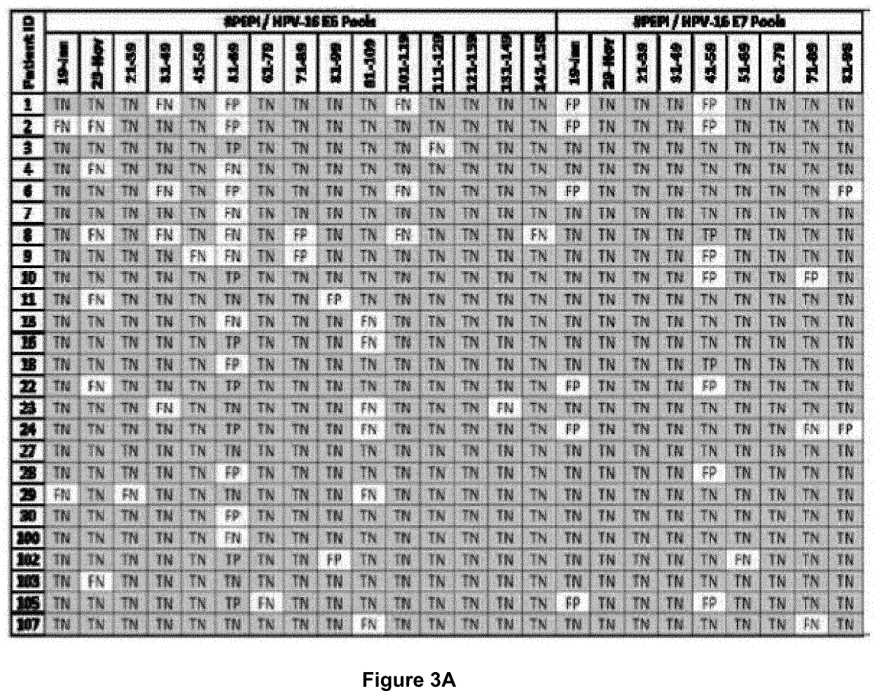

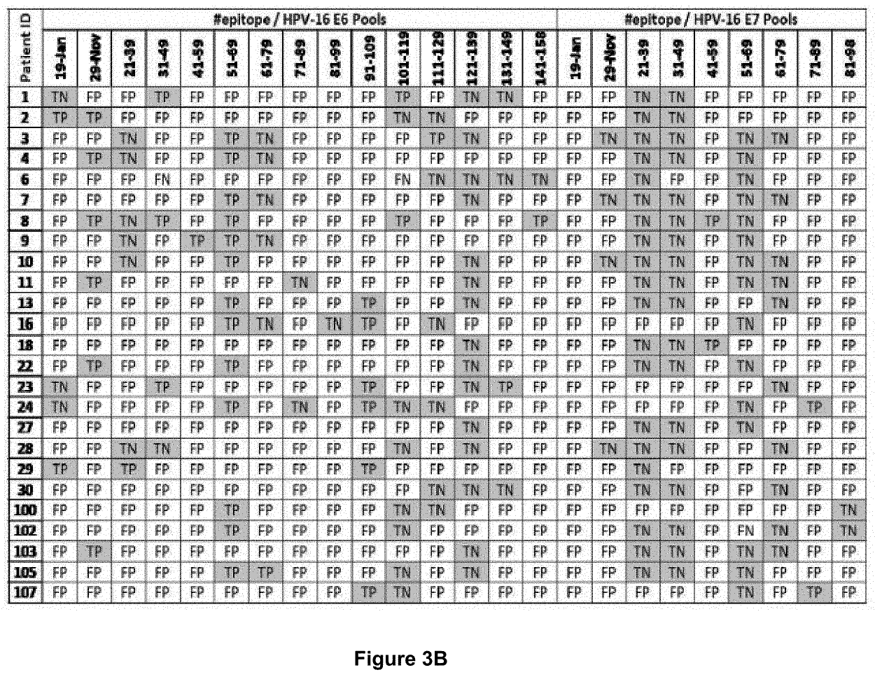

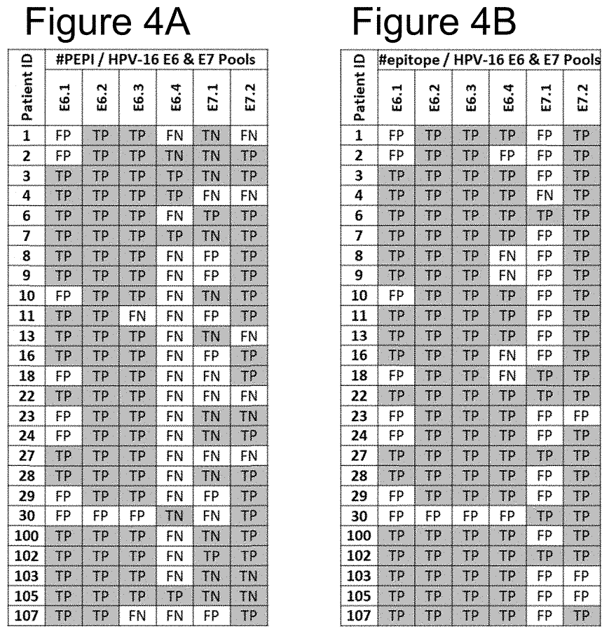

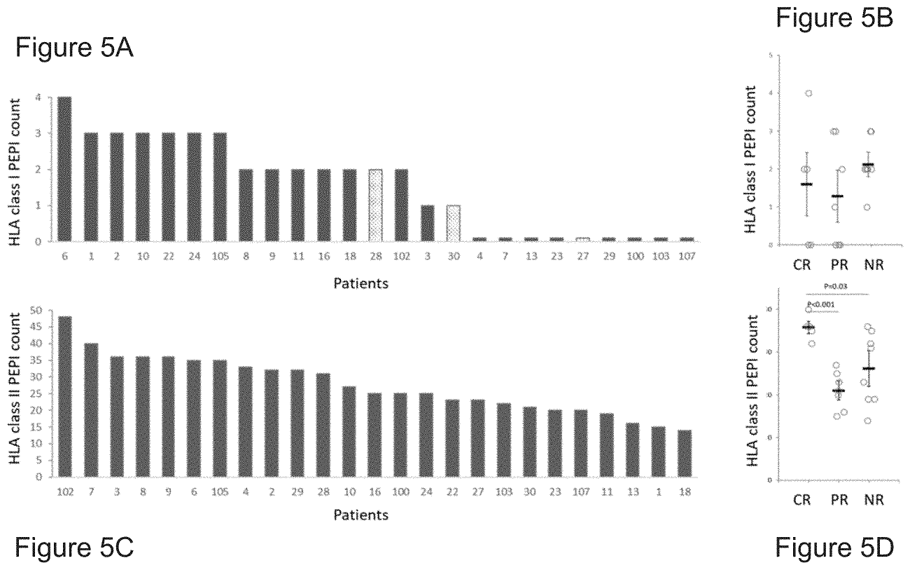

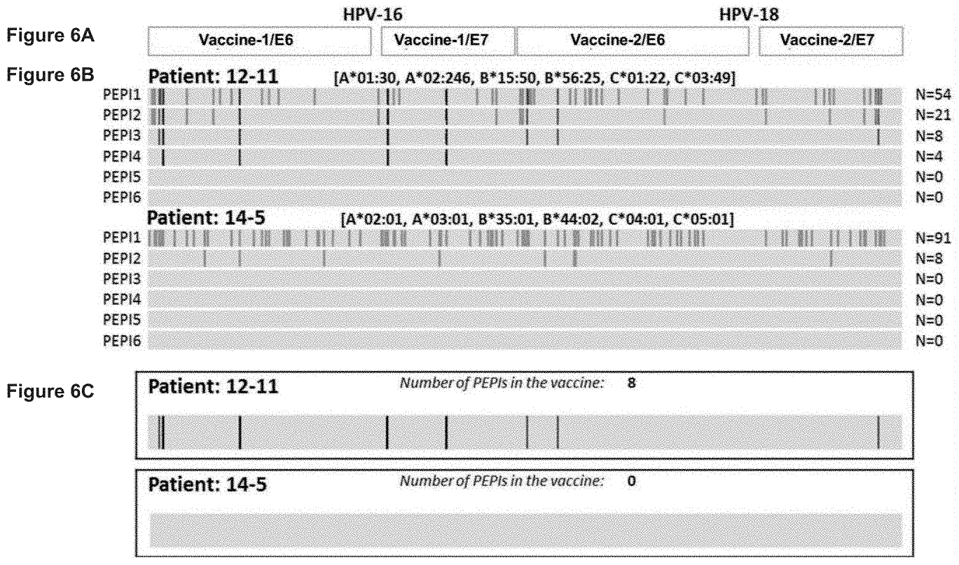

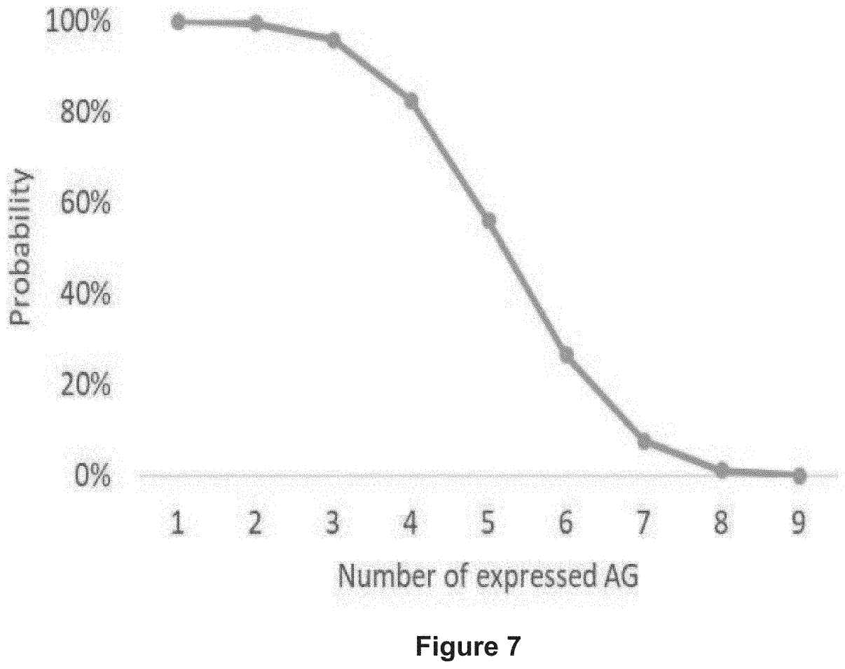

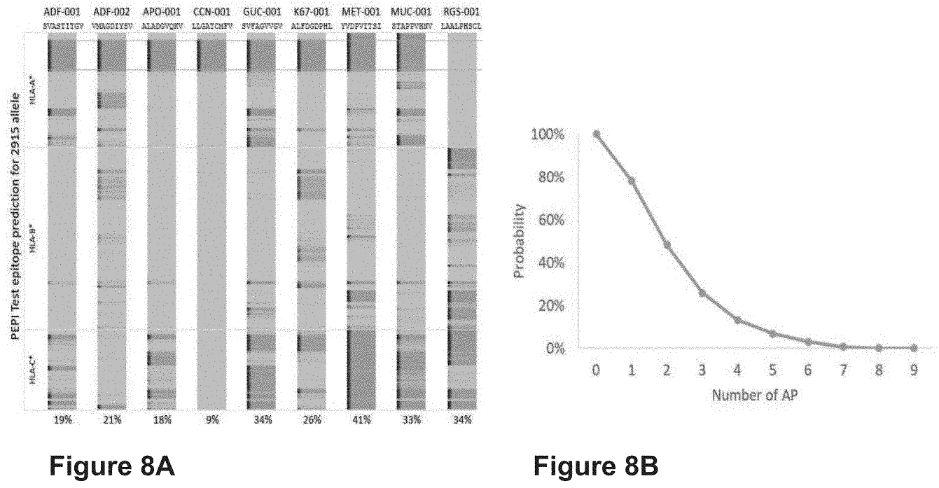

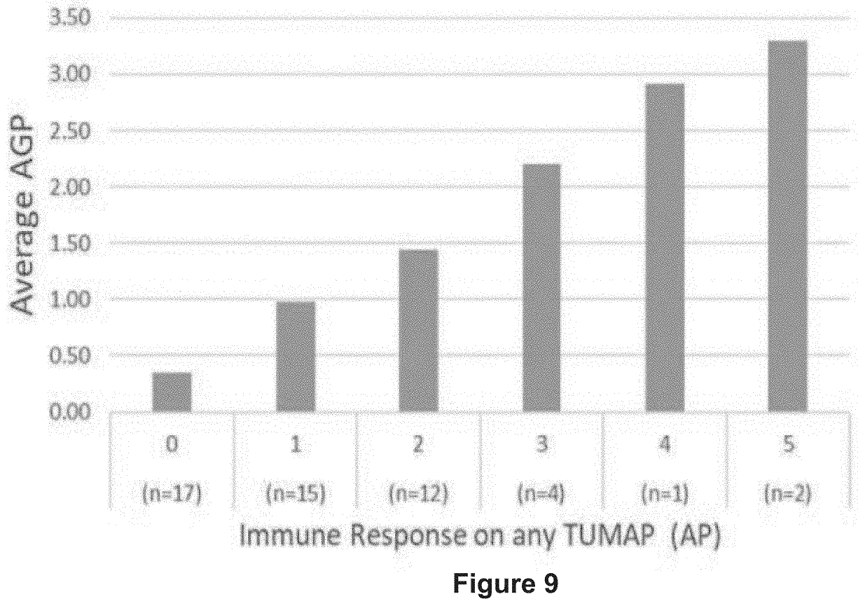

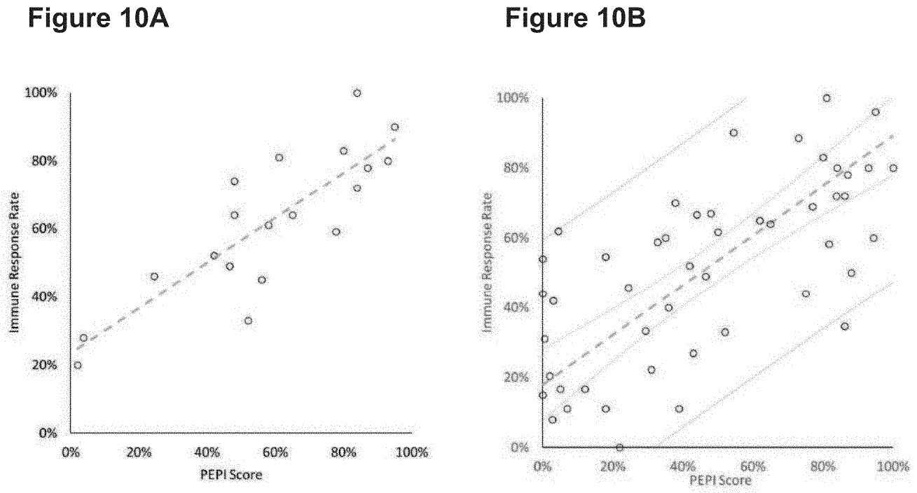

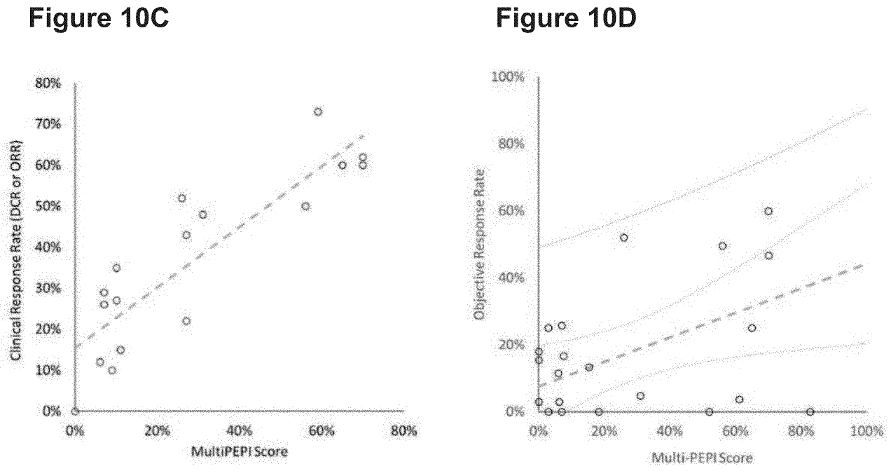

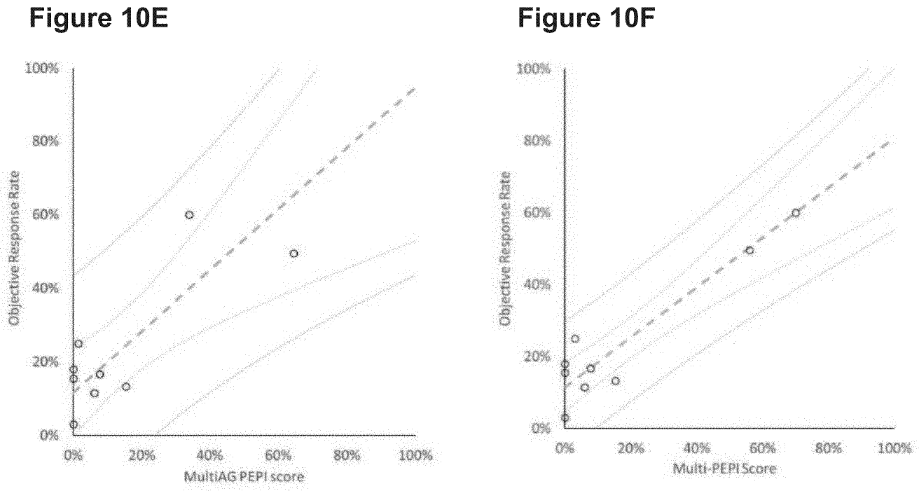

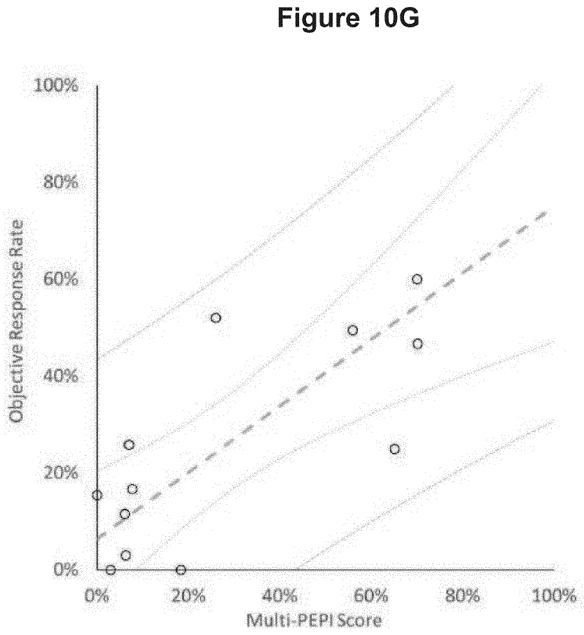

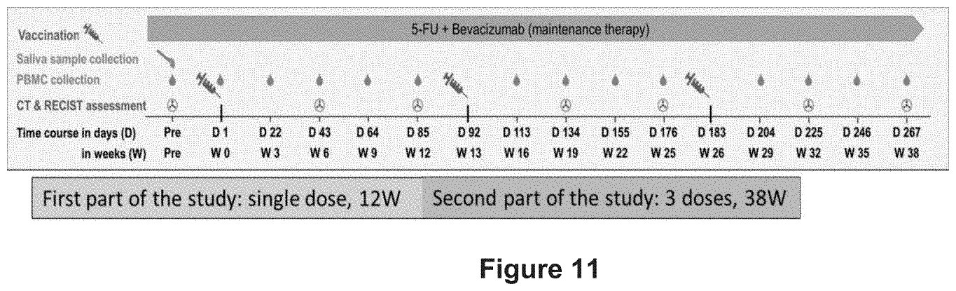

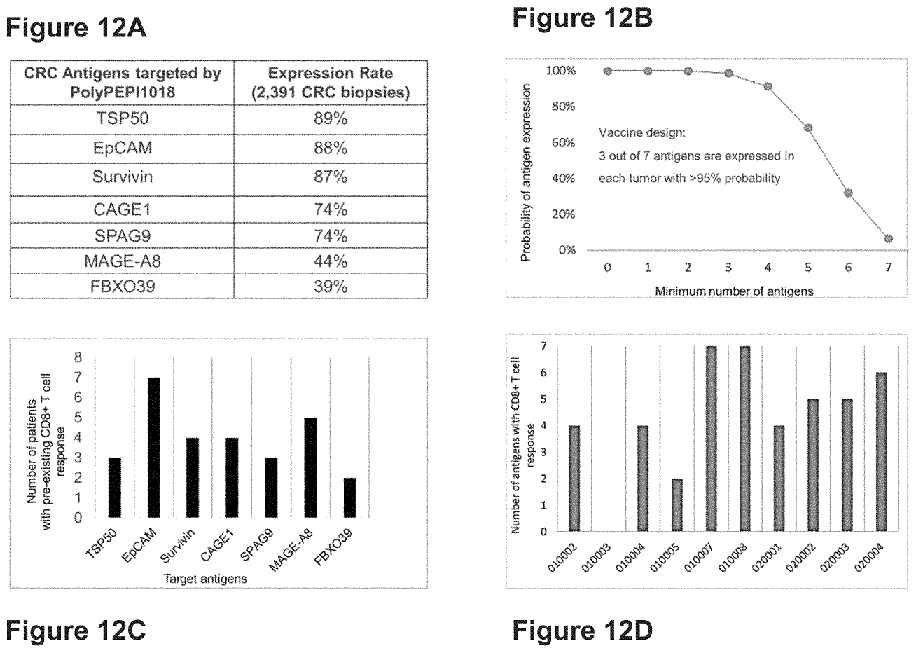

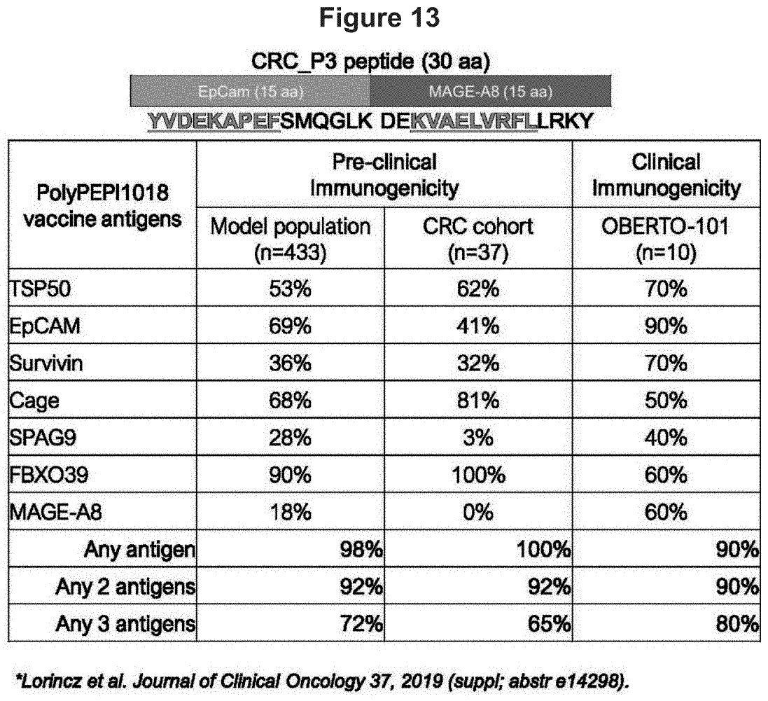

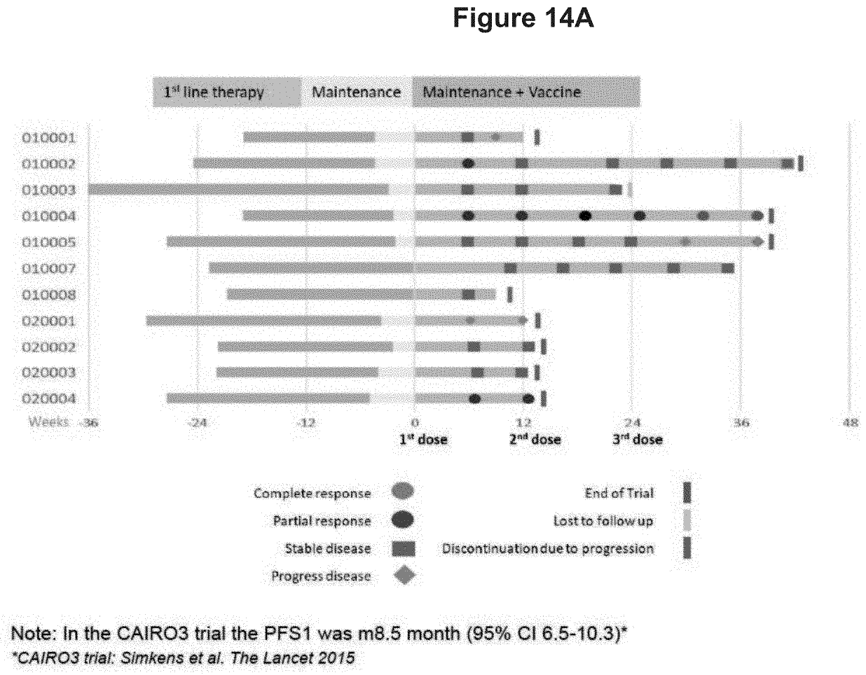

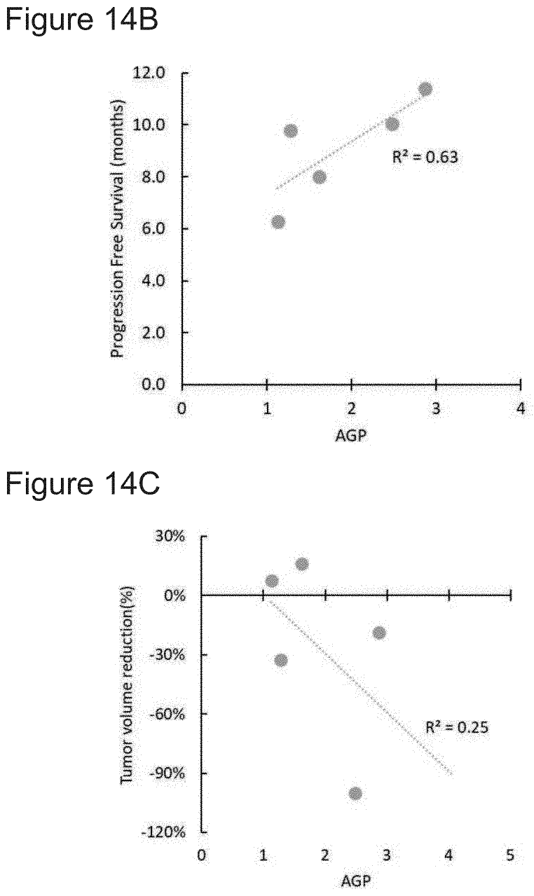

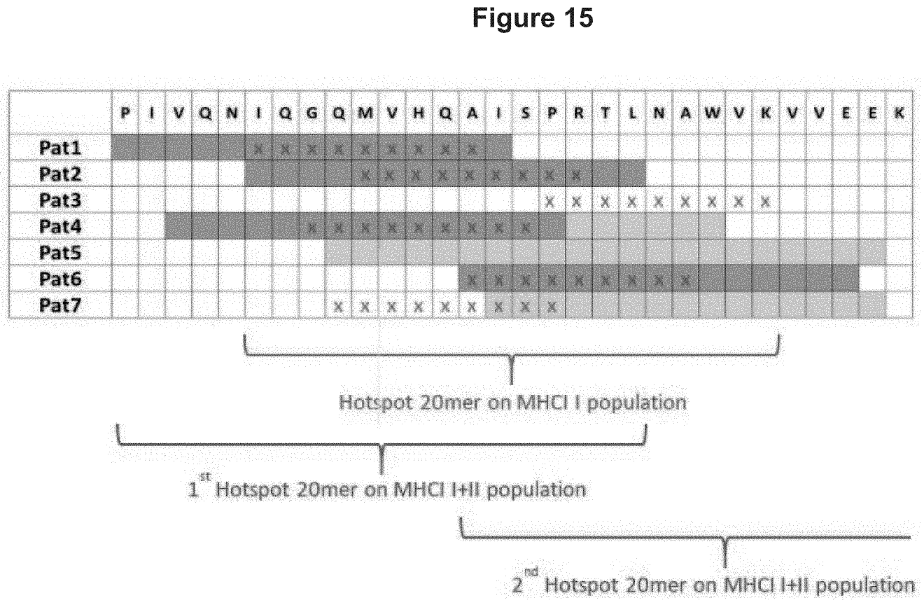

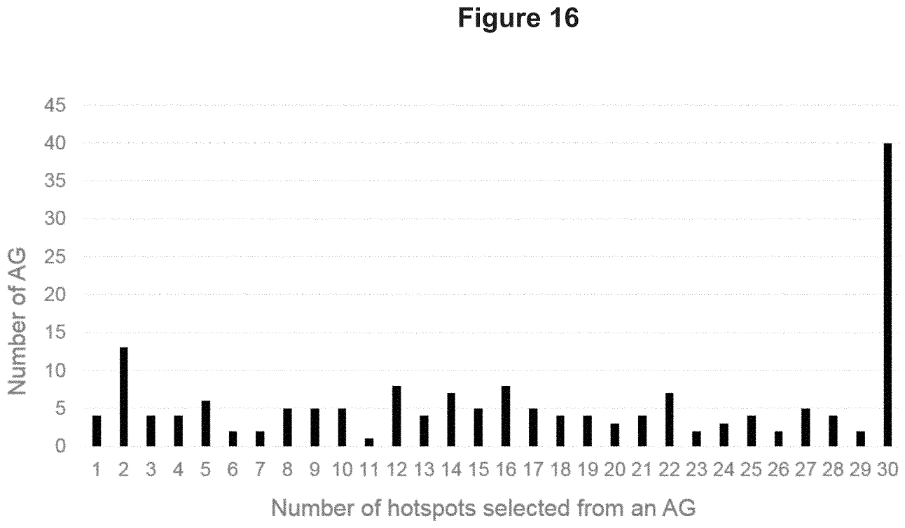

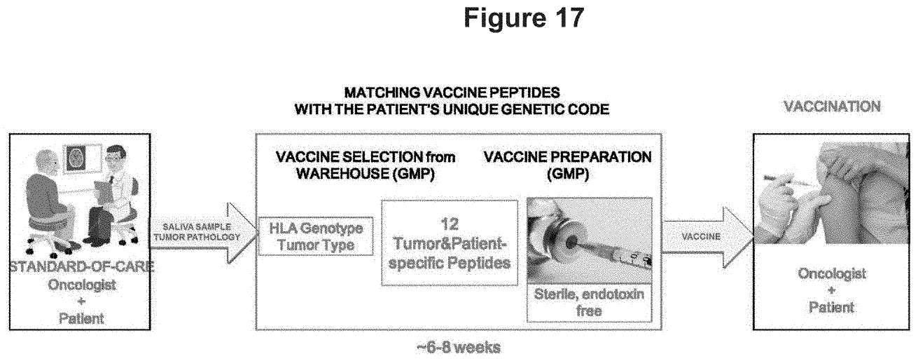

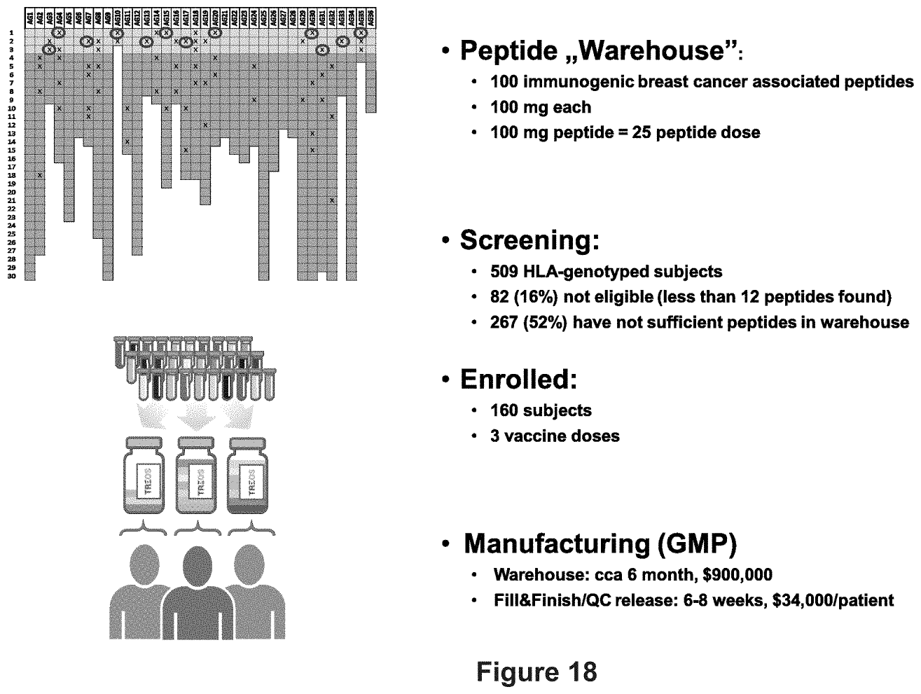

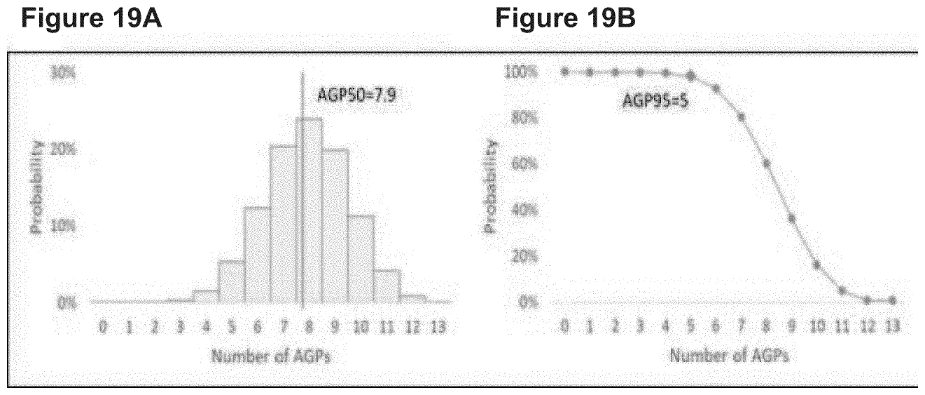

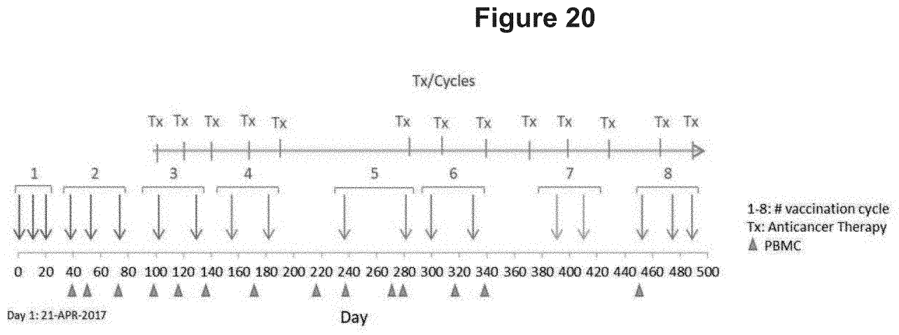

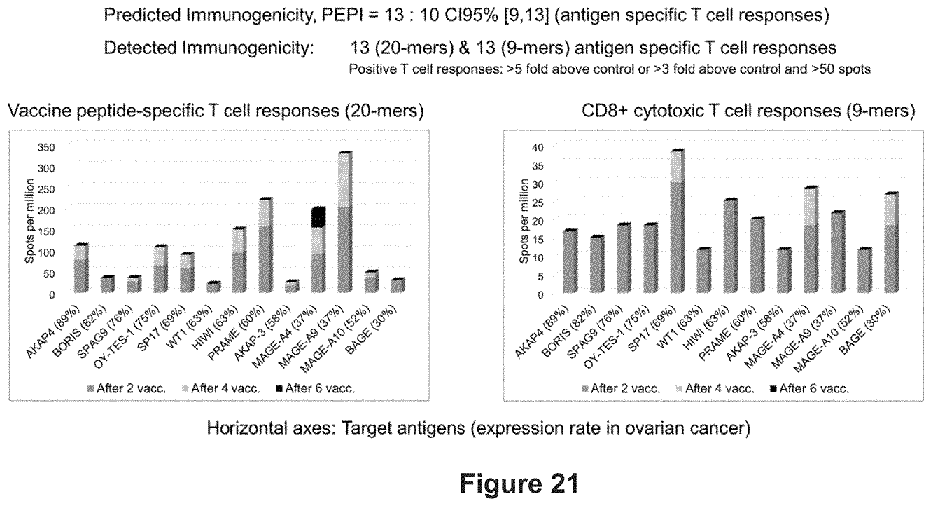

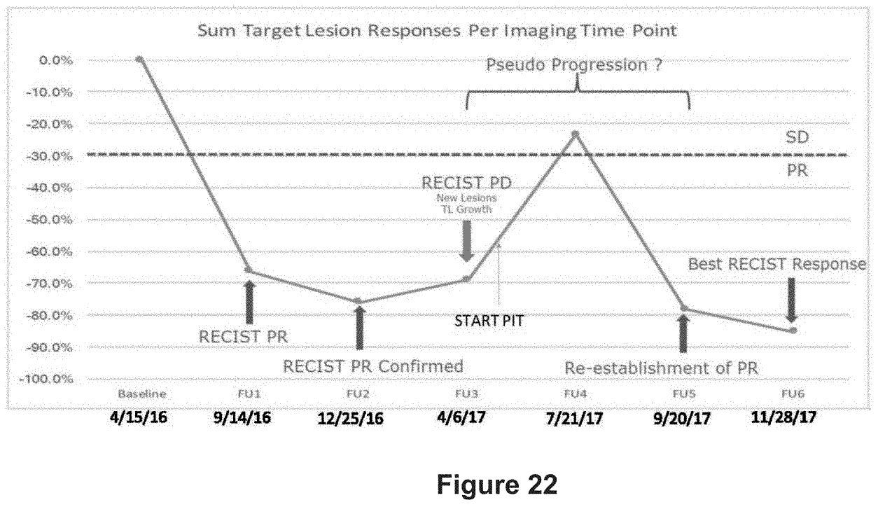

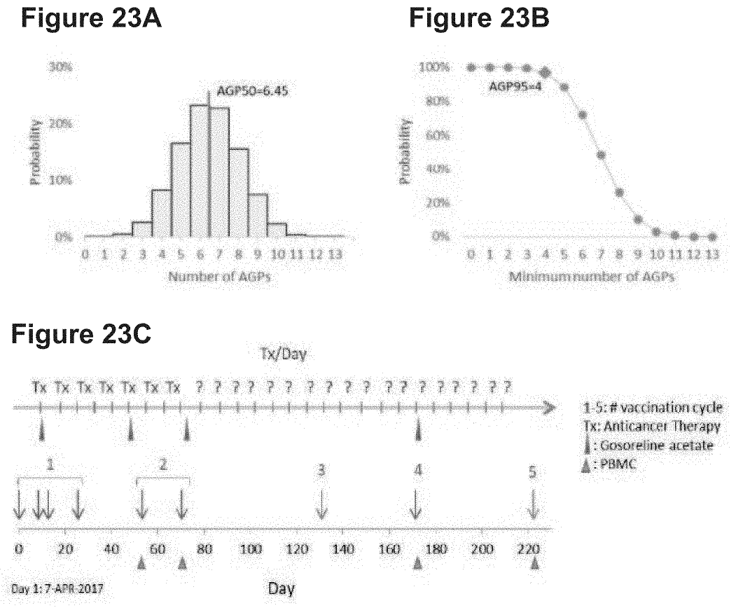

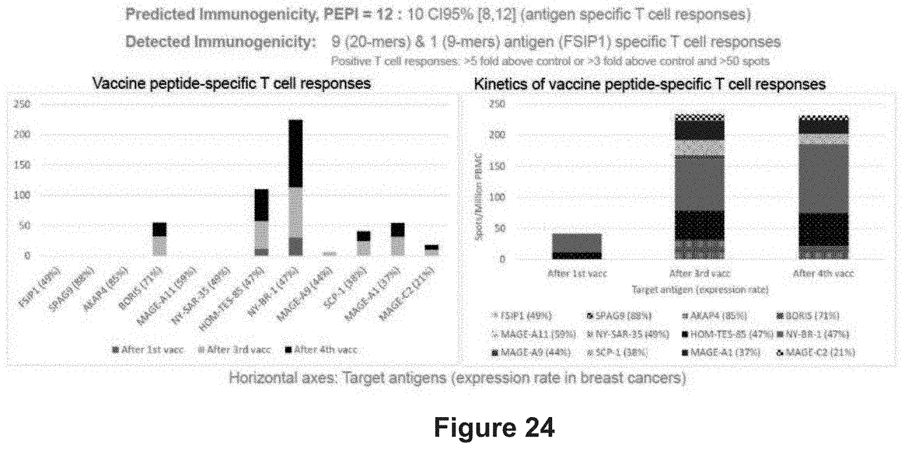

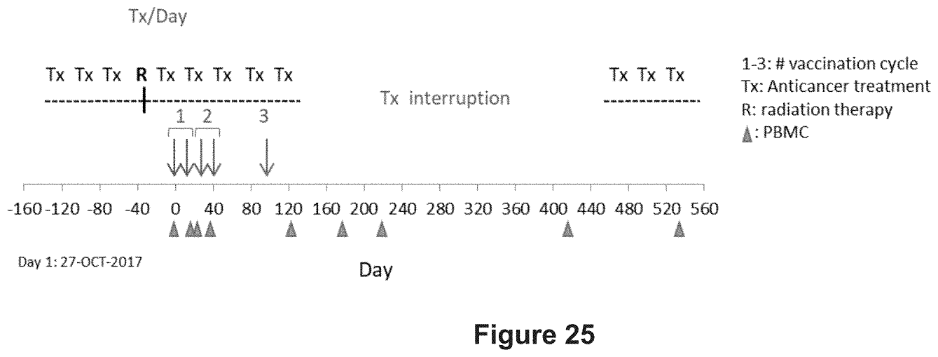

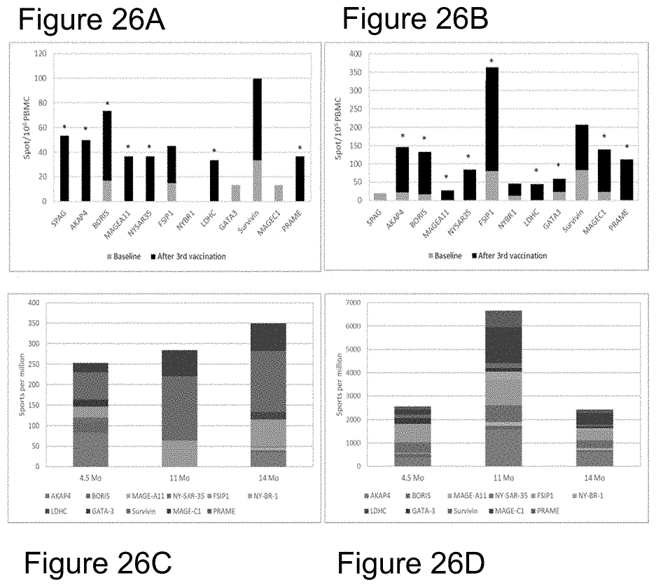

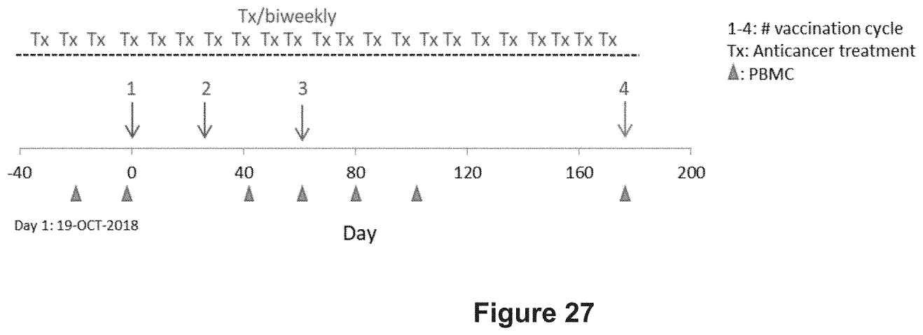

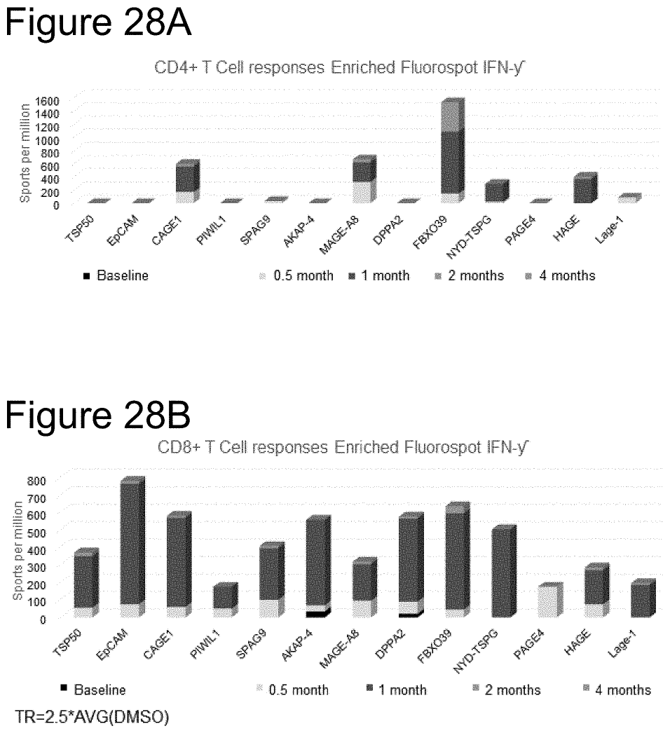

Distribution of HLA class I PEPI3+ compared to CD8+ T cell responses measured by a state of art assay among peptide pools used in the CD8+ T cell response assays. FIG. 3A: HLA class I restricted PEPI3+s. The 90% Overall Percent of Agreement (OPA) among the T cell responses and PEPI3+ peptides demonstrate the utility of the invented peptides for prediction of vaccine induced T cell response set of individuals (p<0.001). FIG. 3B: Class I HLA restricted epitopes (PEPI1+). The OPA between predicted epitopes and CD8+ T cell responses was 25% (not statistically significant). True positive (TP), both peptide and T cell responses were detected (shaded); True negative (TN): neither peptides nor T cell responses were detected (shaded); False negative (FN), only T cell responses were detected; False positive (FP), only peptide were detected.

FIGS. 4A-B

Correlation between PEPI Test predicted CD4 peptides and T-cell reactivity measured with peptide pools in patients treated with SLP vaccine. FIG. 4A: ≥3 HLA class II allele-binding PEPIs; FIG. 4B: single HLA class II allele-binding epitopes. Gray: true positive (TP) and true negative (TN) responses; White: false negative (FN) and false positive (FP) responses. TP: both peptide and T cell responses were detected; TN: neither peptides nor T cell responses were detected; FN: only T cell responses were detected; FP: only peptides were detected.

FIGS. 5A-D

Multiple HLA binding peptides that define the HPV-16 LPV vaccine specific T cell response set of 20 VIN-3 and 5 cervical cancer patients. PEPI counts were compared to clinical responses after treatment with LPV. Predicted CD8+ T cell responders according to HLA class I PEPIs (FIG. 5A) and CD4+ T cell responders according to HLA class II PEPIs (FIG. 5B). Correlation between HLA class I (FIG. 5C) and class II (FIG. 5D) PEPI count and clinical response at 3 months follow-up in VIN-3 patients. Predicted T cell responders: PEPI count ≥1. Gray column, patient with HPV16 E6- and/or E7-specific T cell response; Dashed column, patient without T cell responses. CR, complete clinical responder; PR, partial clinical responder; NR, clinical non-responder.

FIGS. 6A-C

The multiple HLA class I binding peptides that define the HPV vaccine specific T cell response set of 2 patients. FIG. 6A: Four HPV antigens in the HPV vaccine. Boxes represent the length of the amino acid sequences from the N terminus to the C terminus. FIG. 6B: Process to identify the multiple HLA binding peptides of two patients: HLA sequences of the patients labelled as 4-digit HLA genotype right from the patient's ID. The location of the 1^{st} amino acid of the 54 and 91 epitopes that can bind to the patient 12-11 and patient 14-5 HLAs (PEPI1+) respectively are depicted with lines. PEPI2 represents the peptides selected from PEPI1+s that can bind to multiple HLAs of a patient (PEPI2+). PEPI3 represent peptides that can bind to ≥3 HLAs of a patient (PEPI3+). PEPI4 represent peptides that can bind to ≥4 HLAs of a patient (PEPI4+). PEPIS represent peptides that can bind to ≥5 HLAs of a patient (PEPI5+). PEPI6 represent peptides that can bind to 6 HLAs of a patient (PEPI6). FIG. 6C: The DNA vaccine specific PEPI3+ set of two patients characterizes their vaccine specific T cell responses.

FIG. 7

TSA expression probability targeted by IMA901 vaccine.

FIGS. 8A-B

HLA Class I allele binding properties of TUMAPs of IMA901 peptide vaccine for 2,915 common alleles. (FIG. 8A) and for the Class I genotype (6 alleles) of 51 HLA-A*02+ RCC patients. Percentages at the bottom indicate the proportion of HLAs the TUMAPs can bind to. Lines in darker grey indicate binding HLA alleles. (FIG. 8B) Probability indicates the proportion of patients who can present the indicated number of TUMAPs with their three or more HLAs. AP indicates number of antigens which can generate at least one PEPI. In this case, since both the antigens and the predicted PEPIs are 9 mers (SEQ ID NOS 5958-5966, respectively, in order of appearance), AP=TUMAP=PEPI.

FIG. 9

Correlation between immune response measured for any TUMAP and immune response against expressed antigen on the tumor (AGP).

FIGS. 10A-G

Correlation study between immune response rates (IRR) and PEPI Score, between objective response rates (ORR) and MultiPEPI Scores and between objective response rates (ORR) and MultiAg PEPI Scores. FIG. 10A: Preliminary experiment to explore the relationship between PEPI Score and immune response rate of therapeutic vaccines (r²=0.7, p=0.001). FIG. 10B: IRR—PEPI Score plot. (r²=0.47, p=0.001). FIG. 10C: MultiPEPI Score and clinical response rate of therapeutic vaccines (r²=0.75, p=0.001). FIG. 10D: ORRs plotted against the MultiPEPI Score (r²=0.12, p=0.124). FIG. 10E: ORRs plotted against the MultiAg PEPI Score for vaccines with multiple antigens (r²=0.64; p=0.009). F: ORRs plotted against the MultiPEPI Score for vaccines with multiple antigens (r²=0.87; p=0.0002). FIG. 10G: ORRs plotted against the MultiPEPI Score in patients with target antigen positive disease (r²=0.56 and p=0.005). Dark grey dashed lines indicate the 95% confidence interval; light grey dashed line indicates the trendline.

FIG. 11

OBERTO trial design (NCT03391232)

FIGS. 12A-D

Antigen expression in CRC cohort of OBERTO trial (n=10). FIG. 12A: Expression frequencies of PolyPEPI1018 source antigens determined based on 2391 biopsies. FIG. 12B: PolyPEPI1018 vaccine design specified as 3 out of 7 TSAs are expressed in CRC tumors with above 95% probability. FIG. 12C: In average, 4 out of the 10 patients had pre-existing immune responses against each target antigens, referring to the real expression of the TSAs in the tumors of the patients. FIG. 12D: 7 out of the 10 patients had pre-existing immune responses against minimum of 1 TSA, in average against 3 different TSAs.

FIG. 13

Immunogenicity of PolyPEPI1018 in CRC patients confirms proper target antigen and target peptide selection. Upper part: target peptide selection and peptide design of PolyPEPI1018 vaccine composition (SEQ ID NO: 5967). Two 15 mers from CRC specific CTA (TSA) selected to contain 9 mer PEPI3+ predominant in representative Model population. Table: PolyPEPI1018 vaccine has been retrospectively tested during a preclinical study in a CRC cohort and was proven to be immunogenic in all tested individuals for at least one antigen by generating PEPI3+s. Clinical immune responses were measured specific for at least one antigen in 90% of patients, and multi-antigen immune responses were also found in 90% of patients against at least 2, and in 80% of patients against at least 3 antigens as tested with IFNy fluorospot assay specifically measured for the vaccine-comprising peptides.

FIGS. 14A-C

Clinical response for PolyPEPI1018 treatment. FIG. 14A: Swimmer plot of clinical responses of OBERTO trial (NCT03391232). FIG. 14B: Association progression free survival (PFS) and AGP count. FIG. 14C: Association tumour volume and AGP count.

FIG. 15

Illustration of hotspot analysis. Analysis identifies hotspots in sample of 7 patients (Pat1-Pat7) in a peptide of amino acid sequence PIVQNIQGQMVHQAISPRTLNAWVKVVEEK (SEQ ID NO: 5932). Crosses indicate position of a T cell epitope (9 mer) capable of binding to at least three HLA class I alleles (HLA class I-binding PEPI3+). Light shade indicates a T cell epitope (15 mer) capable of binding to at least four HLA class II alleles (HLA class II-binding PEPI4+). Dark shade indicates HLA class II-binding PEPI4+ with an embedded HLA class I-binding PEPI3+. The 20 mer containing a HLA class I-binding PEPI3+ in the maximum number of the 7 patients is indicated. The 20 mer containing HLA class II-binding PEPI4+ with an embedded HLA class I-binding PEPI3+ in the maximum number of the 7 patients is indicated as 1^{st} Hotspot 20 mer. This 1^{st} Hotspot might be selected in a first cycle of a method of the present disclosure. In a second cycle, Pat1, Pat2 and Pat4 may be disregarded and the indicated second Hotspot selected.

FIG. 16

Distribution of hotspot amino acid sequence selection after 30 cycles. Selection of fewer than 30 peptides indicates that no more sequences meeting the HLA-binding criteria (20 mer containing HLA class II-binding PEPI4+ with an embedded HLA class I-binding PEPI3+) could be identified in the model population.

FIG. 17

Process for Personalized Vaccination. Process consists of saliva sample collection and tumor sample collection for tumor pathology. Based on the determined HLA genotype of the patient and tumor type of the patient, 12 tumor and patient specific peptides are selected and personalized vaccine comprising the selected 12 peptides is prepared. Vaccine will be then administered to the patient by the oncologist.

FIG. 18

Feasibility study for a “simulated” Breast Cancer Clinical trial. This example demostrates that >80% of patients could be treated with “patient-specific” vaccine selected from a “Warehouse” of 100 different peptides.

FIGS. 19A-B

Probability of vaccine antigen expression in the Patient-A's tumor cells. There is over 95% probability that 5 out of the 13 target antigens in the vaccine regimen is expressed in the patient's tumor. Consequently, the 13 peptide vaccines together can induce immune responses against at least 5 ovarian cancer antigens with 95% probability ((AGP95) FIG. 19B). It has 84% probability that each peptide will induce immune responses in the Patient-A. AGP50 (FIG. 19A) is the mean (expected value)=7.9 (it is a measure of the effectiveness of the vaccine in attacking the tumor of Patient-A).

FIG. 20

Treatment schedule of Patient-A.

FIG. 21

T cell responses of patient-A. A. Left: Vaccine peptide-specific T cell responses (20-mers). right: CD8+ cytotoxic T cell responses (9-mers). Predicted T cell responses are confirmed by bioassay.

FIG. 22

MRI findings of Patient-A treated with personalised (PIT) vaccine. This late stage, heavily pretreated ovarian cancer patient had an unexpected objective response after the PIT vaccine treatment. These MRI findings suggest that PIT vaccine in combination with chemotherapy significantly reduced her tumor burden. not appear on normal cells of the tissue in which the tumor developed.

FIGS. 23A-C

Probability of vaccine antigen expression in the Patient-B's tumor cells and treatment schedule of Patent-B. FIG. 23A: There is over 95% probability that 4 out of the 13 target antigens in the vaccine is expressed in the patient's tumor. FIG. 23B: Consequently, the 12 peptide vaccines together can induce immune responses against at least 4 breast cancer antigens with 95% probability (AGP95). It has 84% probability that each peptide will induce immune responses in the Patient-B. AGP50=6.45; it is a measure of the effectiveness of the vaccine in attacking the tumor of Patient-B. FIG. 23C: Treatment schedule of Patient-B.

FIG. 24

T cell responses of Patient-A. Left: Vaccine peptide-specific T cell responses (20-mers) of P. Right: Kinetic of vaccine-specific CD8+ cytotoxic T cell responses (9-mers). Predicted T cell responses are confirmed by bioassay.

FIG. 25

Treatment schedule of Patient-C.

FIGS. 26A-D

T cell responses of Patient-C. FIG. 26A: Vaccine peptide-specific T cell responses (20-mers). FIG. 26B: Vaccine peptide-specific CD8+ T cell responses (9-mers). FIGS. 26C-D: Kinetics of vaccine-specific CD4+ T cells and CD8+ cytotoxic T cell responses (9-mers), respectively. Long lasting immune responses both CD4 and CD 8 T cell specific are present after 14 months.

FIG. 27

Treatment schedule of Patient-D.

FIGS. 28A-B

Immune responses of Patient-D for PIT treatment. FIG. 28A: CD4+ specific T cell responses (20 mer) and FIG. 28B: CD8+ T cell specific T cell responses (9 mer). 0.5-4 months refer to the timespan following the last vaccination until PBMC sample collection.

### DESCRIPTION OF THE SEQUENCES

SEQ ID NOs: 1 to 2786 set forth the “hotspot” sequences from cancer antigens described in Table 25A.

SEQ ID NOs: 2787 to 5431 set forth the “hotspot” sequences from cancer antigens described in Table 28.

SEQ ID NOs: 5432 to 5931 set forth the “hotspot” sequences from cancer antigens described in Table 25B.

SEQ ID NO: 5932 sets forth the amino acid sequence shown in FIG. 15.

SEQ ID NOs: 5933 to 5945 set forth sequences of personalized vaccine of Patient-A and are described in Table 31.

SEQ ID NOs: 5946 to 5957 set forth sequences of personalized vaccine of Patient-B and are described in Table 33.

SEQ ID NOs: 5958-5966 set forth the 9 mer sequences shown in FIG. 8.

SEQ ID NO: 5967 sets forth the PolyPEPI1018 vaccine peptide shown in FIG. 13.

### DETAILED DESCRIPTION

### HLA Genotypes

HLAs are encoded by the most polymorphic genes of the human genome. Each person has a maternal and a paternal allele for the three HLA class I molecules (HLA-A*, HLA-B*, HLA-C*) and four HLA class II molecules (HLA-DP*, HLA-DQ*, HLA-DRB1*, HLA-DRB3*/4*/5*). Practically, each person expresses a different combination of 6 HLA class I and 8 HLA class II molecules that present different epitopes from the same protein antigen.

The nomenclature used to designate the amino acid sequence of the HLA molecule is as follows: gene name*allele:protein number, which, for instance, can look like: HLA-A*02:25. In this example, “02” refers to the allele. In most instances, alleles are defined by serotypes—meaning that the proteins of a given allele will not react with each other in serological assays. Protein numbers (“25” in the example above) are assigned consecutively as the protein is discovered. A new protein number is assigned for any protein with a different amino acid sequence (e.g. even a one amino acid change in sequence is considered a different protein number). Further information on the nucleic acid sequence of a given locus may be appended to the HLA nomenclature, but such information is not required for the methods described herein.

The HLA class I genotype or HLA class II genotype of an individual may refer to the actual amino acid sequence of each class I or class II HLA of an individual, or may refer to the nomenclature, as described above, that designates, minimally, the allele and protein number of each HLA gene. In some embodiments, the HLA genotype of an individual is obtained or determined by assaying a biological sample from the individual. The biological sample typically contains subject DNA. The biological sample may be, for example, a blood, serum, plasma, saliva, urine, expiration, cell or tissue sample. In some embodiments the biological sample is a saliva sample. In some embodiments the biological sample is a buccal swab sample. An HLA genotype may be obtained or determined using any suitable method. For example, the sequence may be determined via sequencing the HLA gene loci using methods and protocols known in the art. In some embodiments, the HLA genotype is determined using sequence specific primer (SSP) technologies. In some embodiments, the HLA genotype is determined using sequence specific oligonucleotide (SSO) technologies. In some embodiments, the HLA genotype is determined using sequence based typing (SBT) technologies. In some embodiments, the HLA genotype is determined using next generation sequencing. Alternatively, the HLA set of an individual may be stored in a database and accessed using methods known in the art.

### HLA-Epitope Binding

A given HLA of a subject will only present to T cells a limited number of different peptides produced by the processing of protein antigens in an APC. As used herein, “display” or “present”, when used in relation to HLA, references the binding between a peptide (epitope) and an HLA. In this regard, to “display” or “present” a peptide is synonymous with “binding” a peptide.

As used herein, the term “epitope” or “T cell epitope” refers to a sequence of contiguous amino acids contained within a protein antigen that possesses a binding affinity for (is capable of binding to) one or more HLAs. An epitope is HLA- and antigen-specific (HLA-epitope pairs, predicted with known methods), but not subject specific.

The term “personal epitope”, or “PEPI” as used herein distinguishes a subject-specific epitope from an HLA specific epitope. A “PEPI” is a fragment of a polypeptide consisting of a sequence of contiguous amino acids of the polypeptide that is a T cell epitope capable of binding to one or more HLA class I molecules of a specific human subject. In other words a “PEPI” is a T cell epitope that is recognised by the HLA class I set of a specific individual. In contrast to an “epitope”, PEPIs are specific to an individual because different individuals have different HLA molecules which each bind to different T cell epitopes. In appropriate cases a “PEPI” may also refer to a fragment of a polypeptide consisting of a sequence of contiguous amino acids of the polypeptide that is a T cell epitope capable of binding to one or more HLA class II molecules of a specific human subject.

“PEPI1” as used herein refers to a peptide, or a fragment of a polypeptide, that can bind to one HLA class I molecule (or, in specific contexts, HLA class II molecule) of an individual. “PEPI1+” refers to a peptide, or a fragment of a polypeptide, that can bind to one or more HLA class I molecule of an individual.

“PEPI2” refers to a peptide, or a fragment of a polypeptide, that can bind to two HLA class I (or II) molecules of an individual. “PEPI2+” refers to a peptide, or a fragment of a polypeptide, that can bind to two or more HLA class I (or II) molecules of an individual, i.e. a fragment identified according to a method disclosed herein.

“PEPI3” refers to a peptide, or a fragment of a polypeptide, that can bind to three HLA class I (or II) molecules of an individual. “PEPI3+” refers to a peptide, or a fragment of a polypeptide, that can bind to three or more HLA class I (or II) molecules of an individual.

“PEPI4” refers to a peptide, or a fragment of a polypeptide, that can bind to four HLA class I (or II) molecules of an individual. “PEPI4+” refers to a peptide, or a fragment of a polypeptide, that can bind to four or more HLA class I (or II) molecules of an individual.

“PEPI5” refers to a peptide, or a fragment of a polypeptide, that can bind to five HLA class I (or II) molecules of an individual. “PEPI5+” refers to a peptide, or a fragment of a polypeptide, that can bind to five or more HLA class I (or II) molecules of an individual.

“PEPI6” refers to a peptide, or a fragment of a polypeptide, that can bind to all six HLA class I (or six HLA class II) molecules of an individual.

Generally speaking, epitopes presented by HLA class I molecules are about nine amino acids long. For the purposes of this disclosure, however, an epitope may be more or less than nine amino acids long, as long as the epitope is capable of binding HLA. For example, an epitope that is capable of being presented by (binding to) one or more HLA class I molecules may be between 7, or 8 or 9 and 9 or 10 or 11 amino acids long.

### Table 1. Example Software for Determining Epitope-HLA Binding

Using techniques known in the art, it is possible to determine the epitopes that will bind to a known HLA. Any suitable method may be used, provided that the same method is used to determine multiple HLA-epitope binding pairs that are directly compared. For example, biochemical analysis may be used. It is also possible to use lists of epitopes known to be bound by a given HLA. It is also possible to use predictive or modelling software to determine which epitopes may be bound by a given HLA. Examples are provided in Table 1. In some cases a T cell epitope is capable of binding to a given HLA if it has an IC50 or predicted IC50 of less than 5000 nM, less than 2000 nM, less than 1000 nM, or less than 500 nM.

TABLE 1
Example software for determining epitope-HLA binding

| EPITOPE PREDICTION TOOLS | WEB ADDRESS |
|---|---|
| BIMAS, NIH | www-bimas.cit.nih.gov/molbio/hla_bind/ |
| PPAPROC, Tubingen Univ. | |
| MHCPred, Edward Jenner Inst. of | |
| Vaccine Res. | |
| EpiJen, Edward Jenner Inst. of | http://www.ddg-pharmfac.net/epijen/EpiJen/EpiJen.htm |
| Vaccine Res. | |
| NetMHC, Center for Biological | http://www.cbs.dtu.dk/services/NetMHC/ |
| Sequence Analysis | |
| SVMHC, Tubingen Univ. | http://abi.inf.uni-tuebingen.de/Services/SVMHC/ |
| SYFPEITHI, Biomedical | http://www.syfpeithi.de/bin/MHCServer.dll/ |
| Informatics, Heidelberg | EpitopePrediction.htm |
| ETK EPITOOLKIT, Tubingen | http://etk.informatik.uni-tuebingen.de/epipred/ |
| Univ. | |
| PREDEP, Hebrew Univ. | http://margalit.huji.ac.il/Teppred/mhc-bind/index.html |
| Jerusalem | |
| RANKPEP, MIF Bioinformatics | http://bio.dfci.harvard.edu/RANKPEP/ |
| IEDB, Immune Epitope Database | http://tools.immuneepitope.org/mainlhtml/tcell_tools.html |
| EPITOPE DATABASES | WEB ADDRESS |
| MHCBN, Institute of Microbial | http://www.imtech.res.in/raghava/mhcbn/ |
| Technology, Chandigarh, INDIA | |
| SYFPEITHI, Biomedical | http://www.syfpeithi.de/ |
| Informatics, Heidelberg | |
| AntiJen, Edward Jenner Inst. of | http://www.ddg- |
| Vaccine Res. | pharmfac.net/antijen/AntiJen/antijenhomepage.htm |
| EPIMHC database of MHC | http://immunax.dfci.harvard.edu/epimhc/ |
| ligands, MIF Bioinformatics | |
| IEDB, Immune Epitope Database | http://www.iedb.org/ |

HLA molecules regulate T cell responses. Until recently, the triggering of an immune response to individual epitopes was thought to be determined by recognition of the epitope by the product of single HLA allele, i.e. HLA-restricted epitopes. However, HLA-restricted epitopes induce T cell responses in only a fraction of individuals. Peptides that activate a T cell response in one individual are inactive in others despite HLA allele matching. Therefore, it was previously unknown how an individual's HLA molecules present the antigen-derived epitopes that positively activate T cell responses.

The inventors discovered that multiple HLA expressed by an individual need to present the same peptide in order to trigger a T cell response. Therefore the fragments of a polypeptide antigen (epitopes) that are immunogenic for a specific individual (PEPIs) are those that can bind to multiple class I (activate cytotoxic T cells) or class II (activate helper T cells) HLAs expressed by that individual. This discovery is described in PCT/EP2018/055231, PCT/EP2018/055232, PCT/EP2018/055230, EP 3370065 and EP 3369431.

### Peptides

In some aspects the disclosure provides a peptide that comprises the amino acid sequence of any one of SEQ ID NOs: 1 to 2786 as shown in Table 25A and/or SEQ ID NOs: 5432 to 5931 as show in Table 25B and/or SEQ ID NOs: 2787 to 5431 shown in Table 28. Each of SEQ ID NOs: 1 to 5931 is a 20-mer fragment of a TAA, wherein the fragment comprises at least one HLA class II-binding PEPI4+ and at least one HLA class I-binding PEPI3+ embedded in the HLA class II-binding PEPI4+ in subjects of a model population of ˜16,000 subjects.

The 20-mer fragments were identified as described herein to maximise the number of subjects in the model population that would mount T cell responses to a corresponding TAA in response to administration of at least one peptide comprising one of the 20-mers for each TAA. A panel of peptides each comprising a different one or more of the 20-mer fragments, or a suitable sub-selection thereof, therefore represents an ideal panel of peptides from which to select peptides for use in vaccinating against cancer or providing immunotherapy to treat cancer in individual human subjects.

In some cases the peptides or the panel peptides of the present disclosure may (each) comprise one or more of the sequences of SEQ ID NOs: 1 to 2786 and/or 2787 to 5431 and/or 5432 to 5931 that are fragments of polypeptide antigens associated with one or more specific cancers or types of cancer, such as those of Table 24, or any other described herein. Peptides may be selected from such a panel to treat a corresponding cancer. In some cases the polypeptide antigens may have a minimum expression rate in the cancer, such as being expressed in at least about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% of such cancers. In some cases the polypeptide antigens may be those that are most frequently expressed in the cancer, for example the 50, 45, 40, 35, 30, 25, 20, 15, 10, 9, 8, 7, 6, 5, 4, 3, 2 or 1 most commonly expressed antigens, for example as set out in Table 24.

In some cases the peptides or the panel peptides may (each) comprise peptides that comprise the sequences of SEQ ID NOs: 1 to 2786 and/or 2787 to 5431 and/or 5432 to 5931 that are fragments of a specific polypeptide antigen or family of polypeptide antigens, such as any described herein. Peptides may be selected from such a panel to treat a corresponding cancer that is associated with expression of the antigen.

In some cases the peptides or the panel peptides may (each) comprise peptides that comprise the sequences of SEQ ID NOs: 1 to 2786 and/or 2787 to 5431 and/or 5432 to 5931 that were identified by the inventors as described herein in the first 29, 28, 27, 26, 25, 24, 23, 22, 21, 20, 19, 18, 17, 16, 15, 14, 13, 12, 11, 10, 9, 8, 7, 6, 5, 4, 3, 2 or 1 cycles of the method described herein. The peptides identified in earlier cycles are those that are able to induce T cell responses against the corresponding target antigen in the highest proportion of subjects in the model population.

In some cases the panel of peptides comprises peptides that together comprise any 2, 3, 4, 5, 6, 7, 8, 9, 10, 15, 20, 25, 30, 40 or 50, 100, 200, 300, 400 or 500 of the amino acid sequences of Table 25 or Table 28, or of the amino acid sequences of Table 25 or Table 28 that are a fragment of a TAA that is associated with a cancer selected from those listed in Table 24, and/or that were obtained in the first 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 cycles as described herein.

In some cases the panel comprises or encodes at least two, or at least 3, 4, 5, 6, 7, 8, 9, 10, 11 or 12 amino acid sequence selected from SEQ ID NOs: 1 to 2786 and/or 2787 to 5431 and/or 5432 to 5931, each of which comprises a T cell epitope capable of binding to at least three HLA class I alleles and/or a T cell epitope capable of binding to at least three HLA class II alleles of an individual human subject. Such a panel is a personalised, subject-specific selection of peptides that can be used to induce T cell responses in the specific subject.

In some cases the peptides of the disclosure may be up to 50, 45, 40, 35, 34, 33, 32, 32, 31, 30, 29, 28, 27, 26, 25, 24, 23, 22, 21 or 20 amino acids in length. The peptide comprises or consists of an amino acid sequence selected from any of SEQ ID NO: 1 to 2786 and/or 2787 to 5431 and/or 5432 to 5931, which is a fragment of one or more TAAs as shown in Table 24. In some cases the fragment may comprise or consist of a longer fragment of a TAA of which the sequence of SEQ ID NO: 1 to 2786 and/or 2787 to 5431 and/or 5432 to 5931 is a part. The terms “fragment” or “fragment of a polypeptide” as used herein refer to a string of amino acids or an amino acid sequence typically of reduced length relative to the or a reference polypeptide and comprising, over the common portion, an amino acid sequence identical to the reference polypeptide. Such a fragment according to the disclosure may be, where appropriate, included in a larger polypeptide of which it is a constituent.

In some cases the fragment having the amino acid sequence of any one of SEQ ID NOs: 1 to 2786, or the longer fragment of a TAA comprising the amino acid sequence of any one of SEQ ID NOs: 1 to 2786, is flanked at the N and/or C terminus of the peptide by additional amino acids that are not part of the consecutive sequence of the TAA. In some cases the sequence may be flanked by up to 30 or 25 or 20 or 15 or 10, or 9 or 8 or 7 or 6 or 5 or 4 or 3 or 2 or 1 additional amino acid at the N and/or C terminus.

In some aspects the disclosure provides a polynucleic acid or vector that encodes one or more peptides, wherein the encoded peptides comprise the amino acid sequence of any one of SEQ ID NOs: 1 to 2786 and/or 2787 to 5431 and/or 5432 to 5931 and/or 2787 to 3997, as shown in Tables 25 and 28, or panels thereof. All of the disclosure herein relating to peptides comprising the amino acid sequence of any of SEQ ID NOs: 1 to 2786 and shown in Table 25 (and methods and compositions relating to such peptides) also applies equally to polynucleic acids or vectors encoding one of more peptides comprising the amino acid sequence of any of SEQ ID NOs: 1 to 2786 and/or 2787 to 5431 and/or 5432 to 5931 and/or 2787 to 3997.

### Methods of Designing and Producing Peptides

In some embodiments the disclosure provides methods of designing and preparing one or more peptides, or polynucleotides or vectors that encode peptides, that can optimally be used to induce T cell responses against one or more given polypeptide antigens in a given target population of subjects.

### Target Polypeptides

As used herein, the term “polypeptide” refers to a full-length protein, a portion of a protein, or a peptide characterized as a string of amino acids. As used herein, the term “peptide” refers to a short polypeptide. The peptides are typically between 9, or 10, or 11, or 12, or 13, or 14, or 15 or 16 or 17 or 18 or 19 or 20 and 20, or 21, or 22, or 23, or 24, or 25, or 26, or 27, or 28, or 29, or 30, or 35, or 40, or 45, or 50 amino acid in length. In some cases the peptide is not a 9-mer or a 15-mer. Short peptides may not be processed by antigen presenting cells and therefore bind exogenously to the HLA molecules. Thus, injected short peptides may bind in large numbers to the HLA molecules of all nucleated cells that have surface HLA class I, leading to tolerance. On the other hand polypeptides are not processed as efficiently as long peptides. Accordingly in some cases the peptides may be about 20 or 25 to about 30 or 35 amino acids in length.

The method may comprise the step of selecting one or more target polypeptide antigens. The target polypeptide antigen may be any polypeptide or fragment of a polypeptide against which it is desirable to mount a T cell response in a subject of the target population, for example a CD4+ T cell response or a CD8+ T cell response. Typically the target polypeptide is a polypeptide that is expressed by a pathogenic organism (for example, a bacteria or a parasite), a virus, a cancer cell or other disease-associated cell. In some cases the polypeptide may be present in a sample taken from a subject, such as a subject of the specific or target human population.

The polypeptide may be a Tumor Specific Antigen (TSA) and/or cancer- or tumor-associated antigen (TAA). TAAs are proteins expressed in cancer or tumor cells. Examples of TAAs include new antigens (neoantigens, which are expressed during tumorigenesis and altered from the analogous protein in a normal or healthy cell), products of oncogenes and tumor suppressor genes, overexpressed or aberrantly expressed cellular proteins (e.g. HER2, MUC1), antigens produced by oncogenic viruses (e.g. EBV, HPV, HCV, HBV, HTLV), cancer testis antigens (CTA, e.g. MAGE family, NY-ESO) and cell-type-specific differentiation antigens (e.g. MART-1). TAA sequences may be found experimentally, or in published scientific papers, or through publicly available databases, such as the database of the Ludwig Institute for Cancer Research (www.cta.lncc.br/), Cancer Immunity database (cancerimmunity.org/peptide/) and the TANTIGEN Tumor T cell antigen database (cvc.dfci.harvard.edu/tadb/). Exemplary TAAs are listed in Tables 2 and 22. A TSA is an antigen produced a particular type of tumor that does not appear on normal cells of the tissue in which the tumor developed. TSA include shared antigens, neoantigens, and unique antigens. In some cases the polypeptide is not expressed or is minimally expressed in normal healthy cells or tissues, but is expressed (in those cells or tissues) in a high proportion of (with a high frequency in) subjects having a particular disease or condition, such as a type of cancer or a cancer derived from a particular cell type or tissue. Alternatively, the polypeptide may be expressed at low levels in normal healthy cells, but at high levels (overexpressed) in diseased (e.g. cancer) cells or in subjects having the disease or condition. In some cases the polypeptide is expressed in, or expressed at a high level relative to normal healthy cells or subjects, in at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or more of such individuals, or of a subject-matched human subpopulation or model or target population. For example the population may be matched by ethnicity, geographical location, gender, age, disease, disease type or stage, genotype, and/or expression of one or more biomarkers. Expression frequencies (rates) may be determined from published figures and scientific publications.

In some cases the target polypeptide is a cancer testis antigens (CTA). CTA are not typically expressed beyond embryonic development in healthy cells. In healthy adults, CTA expression is limited to male germ cells that do not express HLAs and cannot present antigens to T cells. Therefore, CTAs are considered expressional neoantigens when expressed in cancer cells. CTA expression is (i) specific for tumor cells, (ii) more frequent in metastases than in primary tumors and (iii) conserved among metastases of the same patient (Gajewski ed. Targeted Therapeutics in Melanoma. Springer New York. 2012).

In some cases the target polypeptide is one that is associated with or expressed by cancer cells or cancer cells of a particular type or cancer of a particular cell type of tissue. In some cases the cancer is a solid tumour. In some cases the cancer is a carcinoma, sarcoma, lymphoma, leukemia, germ cell tumor, or blastoma. The cancer may be a hormone related or dependent cancer (e.g., an estrogen or androgen related cancer) or a non-hormone related or dependent cancer. The tumor may be malignant or benign. The cancer may be metastatic or non-metastatic. The cancer may or may not be associated with a viral infection or viral oncogenes. In some cases the cancer is one or more selected from melanoma, lung cancer, renal cell cancer, colorectal cancer, bladder cancer, glioma, head and neck cancer, ovarian cancer, non-melanoma skin cancer, prostate cancer, kidney cancer, stomach cancer, liver cancer, cervix uteri cancer, oesophagus cancer, non-Hodgkin lymphoma, leukemia, pancreatic cancer, corpus uteri cancer, lip cancer, oral cavity cancer, thyroid cancer, brain cancer, nervous system cancer, gallbladder cancer, larynx cancer, pharynx cancer, myeloma, nasopharynx cancer, Hodgkin lymphoma, testis cancer, breast cancer, gastric cancer, colorectal cancer, renal cell cancer, hepatocellular cancer, pediatric cancer and Kaposi sarcoma.

The polypeptide may be a viral protein that is expressed intracellularly. Examples include HPV16 E6, E7; HIV Tat, Rev, Gag, Pol, Env; HTLV-Tax, Rex, Gag, Env, Human herpes virus proteins, Dengue virus proteins. The polypeptide may be a parasite protein that is expressed intracellularly, for example malaria proteins.

Non-limiting examples of suitable polypeptides include those listed in one or more of Tables 2 to 5.

TABLE 2
LIST OF NAMED TUMOUR ANTIGENS WITH CORRESPONDING
ACCESSION NUMBERS.

| | | | | | | | |
|---|---|---|---|---|---|---|---|
| | | 5T4 | Q13641.1 | A1BG | P04217.1 | A33 | Q99795.1 |
| A4GALT | Q9NPC4.1 | AACT | P01011.1 | AAG | Q9M6E9.1 | ABI1 | Q8IZP0.1 |
| ABI2 | Q9NYB9.1 | ABL1 | P00519.1 | ABL-BCR | Q8WUG5.1 | ABLIM3 | O94929.1 |
| ABLL | P42684.1 | ABTB1 | Q969K4.1 | ACACA | Q13085.1 | ACBD4 | Q8NC06.1 |
| ACO1 | P21399.1 | ACRBP | Q8NEB7.1* | ACTL6A | O96019.1 | ACTL8 | Q9H568.1* |
| ACTN4 | O43707.1 | ACVR1 | Q04771.1 | ACVR1B | P36896.1 | ACVR2B | Q13705.1 |
| ACVRL1 | P37023.1 | ACS2B | Q68CK6.1 | ACSL5 | Q9ULC5.1 | ADAM-15 | Q13444.1 |
| ADAM17 | P78536.1 | ADAM2 | Q99965.1* | ADAM29 | Q9UKF5.1* | ADAM7 | Q9H2U9.1 |
| ADAP1 | O75689.1 | ADFP | Q99541.1 | ADGRA3 | Q8IWK6.1 | ADGRF1 | Q5T601.1 |
| ADGRF2 | Q8IZF7.1 | ADGRL2 | O95490.1 | ADHFE1 | Q8IWW8.1 | AEN | Q8WTP8.1 |
| AFF1 | P51825.1 | AFF4 | Q9UHB7.1 | AFP | P02771.1 | AGAP2 | Q99490.1 |
| AGO1 | Q9UL18.1 | AGO3 | Q9H9G7.1 | AGO4 | Q9HCK5.1 | AGR2 | O95994.1 |
| AIFM2 | Q9BRQ8.1 | AIM2 | O14862.1 | AKAP-13 | Q12802.1 | AKAP-3 | O75969.1* |
| AKAP-4 | Q5JQC9.1* | AKIP1 | Q9NQ31.1 | AKT1 | P31749.1 | AKT2 | P31751.1 |
| AKT3 | Q9Y243.1 | ALDH1A1 | P00352.1 | ALK | Q9UM73.1 | ALKBH1 | Q13686.1 |
| ALPK1 | Q96QP1.1 | AMIGO2 | Q86SJ2.1 | ANG2 | O15123.1 | ANKRD45 | Q5TZF3.1* |
| ANO1 | Q5XXA6.1 | ANP32A | P39687.1 | ANXA2 | P07355.1 | APC | P25054.1 |
| APEH | P13798.1 | APOA2 | P02652.1 | APOD | P05090.1 | APOL1 | O14791.1 |
| AR | P10275.1 | ARAF | P10398.1 | ARF4L | P49703.1 | ARHGEF5 | Q12774.1 |
| ARID3A | Q99856.1 | ARID4A | P29374.1 | ARL6IP5 | O75915.1 | ARMC3 | B4DXS3.1* |
| ARMC8 | Q8IUR7.1 | ARTC1 | P52961.1 | ABX | Q96QS3.1* | ATAD2 | Q6PL18.1 |
| ATIC | P31939.1 | AURKC | Q9UQB9.1 | AXIN1 | O15169.1 | AXL | P30530.1 |
| BAAT | Q14032.1 | BAFF | Q9Y275.1 | BAGE-1 | Q13072.1* | BAGE-2 | Q86130.1* |
| BAGE-3 | Q86Y29.1* | BAGE-4 | Q86Y28.1 | BAGE-5 | Q86Y27.1* | BAI1 | O14514.1 |
| BAL | P19835.1 | BALF2 | P03227.1 | BALF4 | P03188.1 | BALF5 | P03198.1 |
| BARF1 | P03228.1 | BBRF1 | P03213.1 | BCAN | Q96GW7.1 | BCAP31 | P51572.1 |
| BCL-2 | P10415.1 | BCL2L1 | Q07817.1 | BCL6 | P41182.1 | BCL9 | O00512.1 |
| BCR | P11274.1 | BCRF1 | P03180.1 | BDLF3 | P03224.1 | BGLF4 | P13288.1 |
| BHLF1 | P03181.1 | BHRF1 | P03182.1 | BILF1 | P03208.1 | BILF2 | P03218.1 |
| BIN1 | O00499.1 | BING-4 | O15213.1 | BIRC7 | Q96CA5.1 | BLLF1 | P03200.1 |
| BLLF2 | P03199.1 | BMI1 | P35226.1 | BMLF1 | Q04360.1 | BMPR1B | O00238.1 |
| BMRF1 | P03191.1 | BNLF2a | P0C739.1 | BNLF2b | Q8AZJ3.1 | BNRF1 | P03179.1 |
| BRAF1 | P15056.1 | BRD4 | O60885.1 | BRDT | Q58F21.1* | BRI3BP | Q8WY22.1 |
| BRINP1 | O60477.1 | BRLF1 | P03209.1 | BTBD2 | Q9BX70.1 | BUB1B | O60566.1 |
| BVRF2 | P03234.1 | BXLF1 | P03177.1 | BZLF1 | P03206.1 | C15orf60 | Q7Z4M0.1* |
| CA 12-5 | Q8WXI7.1 | CA 19-9 | Q969X2.1 | CA195 | Q5TG92.1 | CA9 | Q16790.1 |
| CABYR | O75952.1* | CADM4 | Q8NFZ8.1 | CAGE1 | Q8CT20.1* | CALCA | P01258.1 |
| CALR3 | Q96L12.1 | CAN | P35658.1 | CASC3 | O15234.1 | CASC5 | Q8NG31.1* |
| CASP5 | P51878.1 | CASP8 | Q14790.1 | CBFA2T2 | O43439.1 | CBFA2T3 | O75081.1 |
| CBL | P22681.1 | CBLB | Q13191.1 | CC3 | Q9BUP3.1 | CCDC110 | Q8TBZ0.1* |
| CCDC33 | Q8N5R6.1* | CCDC36 | Q8IYA8.1* | CCDC6 | Q16204.1 | CCDC62 | Q6P9F0.1* |
| CCDC68 | Q9H2F9.1 | CCDC83 | Q8IWF9.1* | CCL13 | Q99616.1 | CCL2 | P13500.1 |
| CCL7 | P80098.1 | CCNA1 | P78396.1* | CCNA2 | P20248.1 | CCNB1 | P14635.1 |
| CCND1 | P24385.1 | CCNE2 | O96020.1 | CCNI | Q14094.1 | CCNL1 | Q9UK58.1 |
| CCR2 | P41597.1 | CD105 | P17813.1 | CD123 | P26951.1 | CD13 | P15144.1 |
| CD133 | O43490.1 | CD137 | Q07011.1 | CD138 | P18827.1 | CD157 | Q10588.1 |
| CD16A | P08637.1 | CD178 | P48023.1 | CD19 | P15391.1 | CD194 | P51679.1 |
| CD2 | P06729.1 | CD20 | P11836.1 | CD21 | P20023.1 | CD22 | P20273.1 |
| CD229 | Q9HBG7.1 | CD23 | P06734.1 | CD27 | P26842.1 | CD28 | P10747.1 |
| CD30 | P28908.1 | CD317 | Q10589.1 | CD33 | P20138.1 | CD350 | Q9ULW2.1 |
| CD36 | P16671.1 | CD37 | P11049.1 | CD4 | P01730.1 | CD40 | P25942.1 |
| CD40L | P29965.1 | CD45 | P08575.1 | CD47 | Q08722.1 | CD51 | P06756.1 |
| CD52 | P31358.1 | CD55 | P08174.1 | CD61 | P05106.1 | CD70 | P32970.1 |
| CD74 | P08922.1 | CD75 | P15907.1 | CD79B | P40259.1 | CD80 | P33681.1 |
| CD86 | P42081.1 | CD8a | P01732.1 | CD8b | P10966.1 | CD95 | P25445.1 |
| CD98 | P08195.1 | CDC123 | O75794.1 | CDC2 | P06493.1 | CDC27 | P30260.1 |
| CDC73 | Q6P1J9.1 | CDCA1 | Q9BZD4.1* | CDCP1 | Q9H5V8.1 | CDH3 | P22223.1 |
| CDK2AP1 | O14519.1 | CDK4 | P11802.1 | CDK7 | P50613.1 | CDKN1A | P38936.1 |
| CDKN2A | P42771.1 | CEA | P06731.1 | CEACAM1 | Q86UE4.1 | CENPK | Q9BS16.1 |
| CEP162 | Q5TB80.1 | CEP290 | O15078.1* | CEP55 | Q53EZ4.1* | CFL1 | P23528.1 |
| CH3L2 | Q15782.1 | CHEK1 | O14757.1 | CK2 | P19784.1 | CLCA2 | Q9UQC9.1 |
| CLOCK | O15516.1 | CLPP | Q16740.1 | CMC4 | P56277.1 | CML66 | Q96RS6.1 |
| CO-029 | P19075.1 | COTL1 | Q14019.1 | COX2 | P35354.1 | COX6B2 | Q6YFQ2.1* |
| CPSF1 | Q10570.1 | CPXCR1 | Q8N123.1* | CREBL2 | O60519.1 | CREG1 | O75629.1 |
| Cripto | P13385.1 | CRISP2 | P16562.1* | *CRK | P46108.1 | CRKL | P46109.1 |
| CRLF2 | Q9HC73.1 | CSAGE | Q6PB30.1 | CT45 | Q5HYN5.1* | CT45A2 | Q5DJT8.1* |
| CT45A3 | Q8NHU0.1* | CT45A4 | Q8N7B7.1* | CT45A5 | Q6NSH3.1* | CT45A6 | P0DMU7.1* |
| CT46 | Q86X24.1* | CT47 | Q5JQC4.1* | CT47B1 | P0C2P7.1* | CTAGE2 | Q96RT6.1* |
| cTAGE5 | O15320.1* | CTCFL | Q8NI51.1* | CTDSP2 | O14595.1 | CTGF | P29279.1 |
| CTLA4 | P16410.1 | CTNNA2 | P26232.1* | CTNNB1 | P35222.1 | CTNND1 | O60716.1 |
| CTSH | P09668.1 | CTSP1 | A0RZH4.1* | CTTN | Q14247.1 | CXCR4 | P61073.1 |
| CXorf48 | Q8WUE5.1* | CXorf61 | Q5H943.1* | Cyclin-E | P24864.1 | CYP1B1 | Q16678.1 |
| CypB | P23284.1 | CYR61 | O00622.1 | CS1 | P28290.1 | CSAG1 | Q6PB30.1* |
| CSDE1 | O75534.1 | CSF1 | P09603.1 | CSF1R | P07333.1 | CSF3R | Q99062.1 |
| CSK | P41240.1 | CSK23 | Q8NEV1.1 | DAPK3 | O43293.1 | DAZ1 | Q9NQZ3.1 |
| DBPC | Q9Y2T7.1 | DCAF12 | Q5T6F0.1* | DCT | P40126.1 | DCUN1D1 | Q96GG9.1 |
| DCUN1D3 | Q8IWE4.1 | DDR1 | Q08345.1 | DDX3X | O00571.1 | DDX6 | P26196.1 |
| CEDE | O75618.1 | DEK | P35659.1 | DENR | O43583.1 | DEPDC1 | Q5TB30.1 |
| DFNA5 | O60443.1 | DGAT2 | Q96PD7.1 | DHFR | P00374.1 | DKK1 | O94907.1 |
| DKK3 | Q9UBP4.1 | DKKL1 | Q9UK85.1* | DLEU1 | O43261.1 | DMBT1 | Q9UGM3.1 |
| DMRT1 | Q9Y5R6.1* | DNAJB8 | Q8NHS0.1* | DNAJC8 | O75937.1 | DNMT3A | Q9Y6K1.1 |
| DPPA2 | Q7Z7J5.1* | DR4 | O00220.1 | DR5 | O14763.1 | DRG1 | Q9Y295.1* |
| DSCR8 | Q96T75.1 | E2F3 | O00716.1 | E2F6 | O75461.1 | E2F8 | A0AVK6.1 |
| EBNA1 | P03211.1 | EBNA2 | P12978.1 | EBNA3 | P12977.1 | EBNA4 | P03203.1 |
| EBNA6 | P03204.1 | EBNA-LP | Q8AZK7.1 | E-cadherin | P12830.1 | ECT2 | Q9H8V3.1 |
| ECTL2 | Q00858.1 | EDAG | Q9BXL5.1* | EEF2 | P13639.1 | EFNA1 | P20827.1 |
| EFS | O43281.1 | EFTUD2 | Q15029.1 | EGFL7 | Q9UHF1.1 | EGFR | p00533.1 |
| E124 | O14681.1 | EIF4EBP1 | Q13541.1 | ELF3 | P78545.1 | ELF4 | Q99607.1 |
| ELOVL4 | Q9GZR5.1* | EMP1 | P54849.1 | ENAH | Q8N8S7.1 | Endosialin | Q9HCU0.1 |
| ENO1 | P06733.1 | ENO2 | P09104.1 | ENO3 | P13929.1 | ENTPD5 | O75356.1 |
| EpCAM | P16422.1 | EPHA2 | P29317.1 | EPHA3 | P29320.1 | EPHB2 | P29323.1 |
| EPHB4 | P54760.1 | EPHB6 | O15197.1 | EPS8 | Q12929.1 | ERBB3 | P21860.1 |
| ERBB4 | Q15303.1 | EREG | O14944.1 | ERG | P11308.1 | ERVK-18 | O42043.1 |
| ERVK-19 | O71037.1 | ESR1 | P03372.1 | ETAA1 | Q9NY74.1 | ETS1 | P14921.1 |
| ETS2 | P15036.1 | ETV1 | P50549.1 | ETV5 | P41161.1 | ETV6 | P41212.1 |
| EV15 | O60447.1 | EWSR1 | Q01844.1 | EYA2 | O00167.1 | EZH2 | Q15910.1 |
| FABP7 | O15540.1 | FAM133A | Q8N9E0.1* | FAM13A | O94988.1 | FAM46D | Q8NEK8.1* |
| FAM58BP | P0C7Q3.1 | FANCG | O15287.1 | FATE1 | Q969F0.1* | FBXO39 | Q8N4B4.1* |
| FBXW11 | Q9UKB1.1 | FCHSD2 | O94868.1 | FER | P16591.1 | FES | P07332.1 |
| FEV | Q99581.1 | FGF10 | O15520.1 | FGF23 | Q9GZV9.1 | FGF3 | P11487.1 |
| FGF4 | P08620.1 | FGF5 | P12034.1 | FGFR1 | P11362.1 | FGFR2 | P21802.1 |
| FGFR3 | P22607.1 | FGFR4 | P22455.1 | FGR | P09769.1 | FLI1 | Q01543.1 |
| FLT3 | P36888.1 | FMNL1 | O95466.1 | FMOD | Q06828.1 | FMR1NB | Q8N0W7.1* |
| FN1 | P02751.1 | Fn14 | Q9NP84.1 | FNIP2 | Q9P278.1 | FOLR1 | P15328.1 |
| FOS | P01100.1 | FosB | P53539.1 | FOSL1 | P15407.1 | FOXM1 | Q08050.1 |
| FOXO1 | Q12778.1 | FOXO3 | O43524.1 | FRAT1 | Q92837.1 | FRMD3 | A2A2Y4.1 |
| FSIP1 | Q8NA03.1 | FSIP2 | Q5CZC0.1 | FSTL3 | O95633.1 | FTHL17 | Q9BXU8.1* |
| FUNDC2 | Q9BWH2.1 | FUS | P35637.1 | FUT1 | P19526.1 | FUT3 | P21217.1 |
| FYN | P06241.1 | GAB2 | Q9UQC2.1 | GADD45G | O95257.1 | GAGE-1 | Q13065.1 |
| GAGE12B | /C/D/E | GAGE12F | P0CL80.1 | GAGE12G | P0CL81.1 | GAGE12H | A6NDE8.1 |
| A1L429.1 | | | | | | | |
| GAGE12I | P0CL82.1 | GAGE12J | A6NER3.1 | GAGE-2 | Q6NT46.1 | GAGE-3 | Q13067.1 |
| GAGE-4 | Q13068.1 | GAGE-5 | Q13069.1 | GAGE-6 | Q13070.1 | GAGE-7 | O76087.1 |
| GAGE-8 | Q9UEU5.1 | GALGT2 | Q00973.1 | GAS7 | O60861.1 | GASZ | Q8WWH4.1 |
| GATA- | P23771.1 | GBU4-5 | Q587J7.1 | GCDFP-15 | P12273.1 | GFAP | P14136.1 |
| GFI1 | Q99684.1 | Ghrelin | Q9UBU3.1 | GHSR | Q92847.1 | GIPC1 | O14908.1 |
| GITR | Q9Y5U5.1 | GKAP1 | Q5VSY0.1 | GLI1 | P08151.1 | Glypican-3 | P51654.1 |
| GML | Q99445.1 | GNA11 | P29992.1 | GNAQ | P50148.1 | GNB2L1 | P63244.1 |
| GOLGA5 | Q8TBA6.1 | gp100 | P40967.1 | gp75 | P17643.1 | Gp96 | P14625.1 |
| GPAT2 | Q6NUI2.1* | GPATCH2 | Q9NW75.1* | GPC-3 | P51654.1 | GPNMB | Q14956.1 |
| GPR143 | P51810.1 | GPR89A | B7ZAQ6.1 | GRB2 | P62993.1 | GRP78 | P11021.1 |
| GUCY1A3 | Q02108.1 | H3F3A | P84243.1 | HAGE | Q9NXZ2.1* | hANP | P01160.1 |
| HBEGF | Q99075.1 | hCG-beta | P01233.1 | HDAC1 | Q13547.1 | HDAC2 | Q92769.1 |
| HDAC3 | O15379.1 | HDAC4 | P56524.1 | HDAC5 | Q9UQL6.1 | HDAC6 | Q9UBN7.1 |
| HDAC7 | Q8WUI4.1 | HDAC8 | Q9BY41.1 | HDAC9 | Q9UKV0.1 | HEATR1 | Q9H583.1 |
| Hepsin | P05981.1 | Her2/neu | P04626.1 | HERC2 | O95714.1 | HERV-K104 | P61576.1 |
| HEXB | P07686.1 | HEXIM1 | O94992.1 | HGRG8 | Q9Y5A9.1 | HIPK2 | Q9H2X6.1 |
| HJURP | Q8NCD3.1 | HMGB1 | P09429.1 | HMOX1 | P09601.1 | HNRPL | P14866.1 |
| HOM-TES-85 | Q9P127.1* | HORMAD1 | Q86X24.1* | HORMAD2 | Q8N7B1.1* | HPSE | Q9Y251.1 |
| HPV16 E6 | P03126.1 | HPV16 E7 | P03129.1 | HPV18 E6 | P06463.1 | HPV18 E7 | P06788.1 |
| HAAS | P01112.1 | HSD17B13 | Q7Z5P4.1 | HSP105 | Q92598.1 | HSP60 | P10809.1 |
| HSPA1A | P08107.1 | HSPB9 | Q9BQS6.1* | HST-2 | P10767.1 | HT001 | Q2TB18.1 |
| hTERT | O14746.1 | HUS1 | O60921.1 | ICAM-1 | P05362.1 | IDH1 | O75874.1 |
| IDO1 | P14902.1 | IER3 | P46695.1 | IGF1R | P08069.1 | IGFS11 | Q5DX21.1* |
| IL13RA2 | Q14627.1* | IMP-3 | Q9NV31.1* | ING3 | Q9NXR8.1 | INPPL1 | O15357.1 |
| INTS6 | Q9UL03.1 | IRF4 | Q15306.1 | IRS4 | O14654.1 | ITGA5 | P08648.1 |
| ITGB8 | P26012.1 | ITPA | Q9BY32.1 | ITPR2 | Q14571.1 | JAK2 | O60674.1 |
| JAK3 | P52333.1 | JARID1B | Q9UGL1.1* | JAZF1 | Q86VZ6.1 | JNK1 | P45983.1 |
| JNK2 | P45984.1 | JNK3 | P53779.1 | JTB | O76095.1 | JUN | P05412.1 |
| JUP | P14923.1 | K19 | P08727.1 | KAAG1 | Q9UBP8.1 | Kallikrein 14 | |
| | | | | | | | Q9P0G3.1 |
| Kallikrein 4 | Q9Y5K2.1 | KAT6A | Q92794.1 | KDM1A | 060341.1 | KDM5A | P29375.1 |
| KIAA0100 | Q14667.1* | KIAA0336 | Q8IWJ2.1 | KIAA1199 | Q8WUJ3.1 | KIAA1641 | A6QL64.1 |
| KIF11 | P52732.1 | KIF1B | O60333.1 | KIF20A | O95235.1 | KIT | P10721.1 |
| KLF4 | O43474.1 | KLHL41 | O60662.1 | KLK10 | O43240.1 | KMT2D | O14686.1 |
| KOC1 | O00425.1 | K-ras | P01116.1 | KRIT1 | O00522.1 | KW-12 | P62913.1 |
| KW-2 | Q96RS0.1 | KW-5 (SEBD4) | Q9HOZ9.1 | KW-7 | O75475.1 | L1CAM | P32004.1 |
| L53 | Q96EL3.1 | L6 | Q9BTT4.1 | LAG3 | P18627.1 | Lage-1 | O75638.1* |
| LATS1 | O95835.1 | LATS2 | Q9NRM7.1 | LCMT2 | O60294.1 | LCP1 | P13796.1 |
| LDHC | P07864.1* | LDLR | P01130.1 | LEMD1 | Q68G75.1* | Lengsin | Q5TDP6.1 |
| LETMD1 | Q6P1Q0.1 | LGALS3BP | Q08380.1 | LGALS8 | O00214.1 | LIN7A | O14910.1 |
| LIPI | Q6XZB0.1* | LIV-1 | Q13433.1 | LLGL1 | Q15334.1 | LMO1 | P25800.1 |
| LMO2 | P25791.1 | LMP1 | P03230.1 | LMP2 | P13285.1 | LOC647107 | Q8TAI5.1* |
| LOXL2 | Q9Y4K0.1 | LRP1 | Q07954.1 | LRRN2 | O75325.1 | LTF | P02788.1 |
| LTK | P29376.1 | LZTS1 | Q9Y250.1 | LY6K | Q17RY6.1* | LYN | P07948.1 |
| LYPD6B | Q8NI32.1* | MAEA | Q7L5Y9.1 | MAEL | Q96JY0.1* | MAF | O75444.1 |
| MAFF | Q9ULX9.1 | MAFG | O15525.1 | MAFK | O60675.1 | MAGE-A1 | P43355.1* |
| MAGE-A10 | P43363.1* | MAGE-A11 | P43364.1* | MAGE-A12 | P43365.1* | MAGE-A2 | P43356.1* |
| MAGE-A2B | Q6P448.1* | MAGE-A3 | P43357.1* | MAGE-A4 | P43358.1* | MAGE-A5 | P43359.1* |
| MAGE-A6 | P43360.1* | MAGE-A8 | P43361.1* | MAGE-A9 | P43362.1* | MAGE-B1 | P43366.1* |
| MAGE-B2 | O15479.1* | MAGE-B3 | O15480.1* | MAGE-B4 | O15481.1* | MAGE-B5 | Q9BZ81.1* |
| MAGE-B6 | Q8N7X4.1* | MAGE-C1 | O60732.1* | MAGE-C2 | Q9UBF1.1* | MAGE-C3 | Q8TD91.1* |
| mammaglobin-A | | MANF | P55145.1 | MAP2K2 | P36507.1 | MAP2K7 | O14733.1 |
| | Q13296.1 | | | | | | |
| MAP3K7 | O43318.1 | MAP4K5 | Q9Y4K4.1 | MARTI | Q16655.1 | MART-2 | Q5VTY9.1 |
| MAS1 | P04201.1 | MC1R | Q01726.1 | MCAK | Q99661.1* | MCF2 | P10911.1 |
| MCF2L | O15068.1 | MCL1 | Q07820.1 | MCTS1 | Q9ULC4.1 | MCSP | Q6UVK1.1 |
| MDK | P21741.1 | MDM2 | Q00987.1 | MDM4 | O15151.1 | ME1 | P48163.1 |
| ME491 | P08962.1 | MECOM | Q03112.1 | MELK | Q14680.1 | MEN1 | O00255.1 |
| MERTK | Q12866.1 | MET | P08581.1 | MFGE8 | Q08431.1 | MFHAS1 | Q9Y4C4.1 |
| MFI2 | P08582.1 | MGAT5 | Q09328.1 | Midkine | P21741.1 | MIF | P14174.1 |
| MK167 | P46013.1 | MLH1 | P40692.1 | MLL | Q03164.1 | MLLT1 | Q03111.1 |
| MLLT10 | P55197.1 | MLLT11 | Q13015.1 | MLLT3 | P42568.1 | MLLT4 | P55196.1 |
| MLLT6 | P55198.1 | MMP14 | P50281.1 | MMP2 | P08253.1 | MMP7 | P09237.1 |
| MMP9 | P14780.1 | MOB3B | Q86TA1.1 | MORC1 | Q86VD1.1* | MPHOSPH1 | Q96Q89.1* |
| MPL | P40238.1 | MRAS | O14807.1 | MRP1 | P33527.1 | MRP3 | O15438.1 |
| MRPL28 | Q13084.1 | MRPL30 | Q8TCC3.1 | MRPS11 | P82912.1 | MSLN | Q13421.1 |
| MTA1 | Q13330.1 | MTA2 | O94776.1 | MTA3 | Q9BTC8.1 | MTCP1 | P56278.1 |
| MTSS1 | O43312.1 | MUC-1 | P15941.1 | MUC-2 | Q02817.1 | MUC-3 | Q02505.1 |
| MUC-4 | Q99102.1 | MUC-5AC | P98088.1 | MUC-6 | Q6W4X9.1 | MUM1 | Q2TAK8.1 |
| MUM2 | Q9Y5R8.1 | MYB | P10242.1 | MYC | P01106.1 | MYCL | P12524.1 |
| MYCLP1 | P12525.1 | MYCN | P04198.1 | MYD88 | Q99836.1 | MYEOV | Q96EZ4.1 |
| MYO1B | O43795.1 | NA88-A | P0C5K6.1* | NAE1 | Q13564.1 | Napsin-A | O96009.1 |
| NAT6 | Q93015.1 | NBAS | A2RRP1.1 | NBPF12 | Q5TAG4.1 | NCOA4 | Q13772.1 |
| NDC80 | O14777.1 | NDUFC2 | O95298.1 | Nectin-4 | Q96NY8.1 | NEK2 | P51955.1 |
| NEMF | O60524.1 | NENF | Q9UMX5.1 | NEURL1 | O76050.1 | NFIB | O00712.1 |
| NFKB2 | Q00653.1 | NF-X1 | Q12986.1 | NFYC | Q13952.1 | NGAL | P80188.1 |
| NGEP | Q6IWH7.1 | NKG2D-L1 | Q9BZM6.1 | NKG2D-L2 | Q9BZM5.1 | NKG2D-L3 | Q9BZM4.1 |
| NKG2D-L4 | Q8TD07.1 | NKX3.1 | Q99801.1 | NLGN4X | Q8N0W4.1 | NLRP4 | Q96MN2.1* |
| NNMT | P40261.1 | NOL4 | O94818.1* | NOTCH2 | Q04721.1 | NOTCH3 | Q9UM47.1 |
| NOTCH4 | Q99466.1 | NOV | P48745.1 | NPM1 | P06748.1 | NR6A1 | Q15406.1* |
| N-RAS | P01111.1 | NRCAM | Q92823.1 | NRP1 | O14786.1 | NSE1 | Q96KN4.1 |
| NSE2 | Q96KN1.1 | NTRK1 | P04629.1 | NUAK1 | O60285.1 | NUGGC | Q68CJ6.1 |
| NXF2 | Q9GZY0.1* | NXF2B | Q5JRM6.1* | NY-BR-1 | Q9BXX3.1 | NYD-TSPG | Q9BWV7.1 |
| NY-ESO-1 | P78358.1* | NY-MEL-1 | P57729.1 | OCA2 | Q04671.1 | ODF1 | Q14990.1* |
| ODF2 | Q5BJF6.1* | ODF3 | Q96PU9.1* | ODF4 | Q2M2E3.1* | OGG1 | O15527.1 |
| OGT | O15294.1 | OIP5 | O43482.1* | OS9 | Q13438.1 | OTOA | Q05BM7.1* |
| OX40 | P43489.1 | OX40L | P23510.1 | P53 | P04637.1 | P56-LCK | P06239.1 |
| PA2G4 | Q9UQ80.1 | PAGE1 | O75459.1* | PAGE2 | Q7Z2X2.1* | PAGE2B | Q5JRK9.1* |
| PAGE3 | Q5JUK9.1* | PAGE4 | 060829.1* | PAGE5 | Q96GU1.1* | PAK2 | Q13177.1 |
| PANO1 | I0J062.1 | PAP | Q06141.1 | PAPOLG | Q9BWT3.1 | PARK2 | O60260.1 |
| PARK7 | Q99497.1 | PARP12 | Q9H0J9.1 | PASD1 | Q8IV76.1* | PAX3 | P23760.1 |
| PAX5 | Q02548.1 | PBF | P00751.1 | PBK | Q96K35.1* | PBX1 | P40424.1 |
| PCDC1 | Q15116.1 | PCM1 | Q15154.1 | PCNXL2 | A6NKB5.1 | PDGFB | P01127.1 |
| PDGFRA | P16234.1 | PEPP2 | Q9HAU0.1* | PGF | P49763.1 | PGK1 | P00558.1 |
| PHLDA3 | Q9Y5J5.1 | PHLPP1 | O60346.1 | PIAS1 | O75925.1 | PIAS2 | O75928.1 |
| PIK3CA | P42336.1 | PIK3CD | O00329.1 | PIK3R2 | O00459.1 | PIM1 | P11309.1 |
| PIM2 | Q9P1W9.1 | PIM3 | Q86V86.1 | PIR | O00625.1 | PIWIL1 | Q96J94.1* |
| PIWIL2 | Q8TC59.1* | PIWIL3 | Q7Z3Z3.1 | PIWIL4 | Q7Z3Z4.1 | PKN3 | Q6P5Z2.1 |
| PLA2G16 | P53816.1 | PLAC1 | Q9HBJ0.1* | PLAG1 | Q6DJT9.1 | PLEKHG5 | O94827.1 |
| PLK3 | Q9H4B4.1 | PLS3 | P13797.1 | PLVAP | Q9BX97.1 | PLXNB1 | O43157.1 |
| PLXNB2 | O15031.1 | PML | P29590.1 | PML-RARA | Q96QH2.1 | POTEA | Q6S8J7.1* |
| POTEB | Q6S5H4.1* | POTEC | B2RU33.1* | POTED | Q86YR6.1* | POTEE | Q6S8J3.1* |
| POTEG | Q6S5H5.1* | POTEH | Q6S545.1* | PP2A | P63151.1 | PPAPDC1B | Q8NEB5.1 |
| PPFIA1 | Q13136.1 | PPIG | Q13427.1 | PPP2R1B | P30154.1 | PRAME | P78395.1* |
| PRDX5 | P30044.1 | PRKAA1 | Q13131.1 | PRKCI | P41743.1 | PRM1 | P04553.1* |
| PRM2 | P04554.1* | PRMT3 | O60678.1 | PRMT6 | Q96LA8.1 | PDL1 | Q9NZQ7.1 |
| PROM1 | O43490.1 | PRSS54 | Q6PEW0.1* | PRSS55 | Q6UWB4.1* | PRTN3 | P24158.1 |
| PRUNE | Q86TP1.1 | PRUNE2 | Q8WUY3.1 | PSA | P07288.1 | PSCA | D3DWI6.1 |
| PSMA | Q04609.1 | PSMD10 | O75832.1 | PSGR | Q9H255.1 | PSP-94 | Q1L6U9.1 |
| PTEN | P60484.1 | PTH-rP | P12272.1 | PTK6 | Q13882.1 | PTPN20A | Q4JDL3.1* |
| PTPRK | Q15262.1 | PTPRZ | P23471.1 | PTTG-1 | O95997.1 | PTTG2 | Q9NZH5.1 |
| PTTG3 | Q9NZH4.1 | PXDNL | A1KZ92.1 | RAB11FIP3 | O75154.1 | RAB8A | P61006.1 |
| RAD1 | O60671.1 | RAD17 | O75943.1 | RAD51C | O43502.1 | RAF1 | P04049.1 |
| RAGE-1 | Q9UQ07.1 | RAP1A | P62834.1 | RARA | P10276.1 | RASSF10 | A6NK89.1 |
| RB1 | P06400.1 | RBL2 | Q08999.1 | RBM46 | Q8TBY0.1* | RBP4 | P02753.1 |
| RCAS1 | O00559.1 | RCVRN | P35243.1 | RECQL4 | 094761.1 | RET | P07949.1 |
| RGS22 | Q8NE09.1* | RGS5 | O15539.1 | RHAMM | O75330.1 | RhoC | P08134.1 |
| RHOXF2 | Q9BQY4.1 | RL31 | P62888.1 | RNASET2 | O00584.1 | RNF43 | Q68DV7.1 |
| RNF8 | O76064.1 | RON | Q04912.1 | ROPN1A | Q9HAT0.1* | ROR1 | Q01973.1 |
| RPA1 | O95602.1 | RPL10A | P62906.1 | RPL7A | P62424.1 | RPS2 | P15880.1 |
| RPS6KA5 | O75582.1 | RPSA | P08865.1 | RQCD1 | Q92600.1* | RRAS2 | P62070.1 |
| RSL1D1 | O76021.1 | RTKN | Q9BST9.1 | RUNX1 | Q01196.1 | RUNX2 | Q13950.1 |
| RYK | P34925.1 | SAGE1 | Q9NXZ1.1* | SART2 | Q9UL01.1 | SART3 | Q15020.1 |
| SASH1 | O94885.1 | sCLU | P10909.1 | SCRN1 | Q12765.1 | SDCBP | O00560.1 |
| SBF-1 | P48061.1 | SDHD | 014521.1 | SEC31A | O94979.1 | SEC63 | Q9UGP8.1 |
| Semaphorin 4D | | SEMG1 | P04279.1* | SFN | P31947.1 | SH2B2 | O14492.1 |
| | Q92854.1 | | | | | | |
| SH2D1B | O14796.1 | SH3BP1 | Q9Y3L3.1 | SHB | Q15464.1 | SHC3 | Q92529.1 |
| SIRT2 | Q8IXJ6.1 | SIVA1 | 015304.1 | SKI | P12755.1 | SLBP | A9UHW6.1 |
| SLC22A10 | Q63ZE4.1 | SLC25A47 | Q6Q0C1.1 | SLC35A4 | Q96G79.1 | SLC45A3 | Q96JT2.1 |
| SLC4AlAP | Q9BWU0.1 | SLCO6A1 | Q86UG4.1* | SLITRK6 | Q9H5Y7.1 | Sm23 | P27701.1 |
| SMAD5 | Q99717.1 | SMAD6 | O43541.1 | SMO | Q99835.1 | Smt3B | P61956.1 |
| SNRPD1 | P62314.1 | SOS1 | Q07889.1 | SOX-2 | P48431.1 | SOX-6 | P35712.1 |
| SOX-11 | P35716 .1 | SPA17 | Q15506.1* | SPACA3 | Q8IXA5.1* | SPAG1 | Q07617.1* |
| SPAG17 | Q6Q759.1* | SPAG4 | Q9NPE6.1* | SPAG6 | O75602.1* | SPAG8 | Q99932.1* |
| SPAG9 | O60271.1* | SPANXA1 | Q9NS26.1* | SPANXB | Q9NS25.1* | SPANXC | Q9NY87.1* |
| SPANXD | Q9BXN6.1* | SPANXE | Q8TAD1.1* | SPANXN1 | Q5VSR9.1* | SPANXN2 | Q5MJ10.1* |
| SPANXN3 | Q5MJ09.1* | SPANXN4 | Q5MJ08.1* | SPANXN | 5Q5MJ07.1* | SPATA19 | Q7Z5L4.1* |
| SPEF2 | Q9C093.1* | SPI1 | P17947.1 | SPINLW1 | O95925.1* | SPO11 | Q9Y5K1.1* |
| SRC | P12931.1 | SSPN | Q14714.1 | SSX-1 | Q16384.1* | SSX-2 | Q16385.1* |
| SSX-3 | Q99909.1* | SSX-4 | O60224.1* | SSX-5 | O60225.1* | SSX-6 | Q7RTT6.1* |
| SSX-7 | Q7RTT5.1* | SSX-9 | Q7RTT3.1* | ST18 | O60284.1 | STAT1 | P42224.1 |
| STEAP1 | Q9UHE8.1 | STK11 | Q15831.1 | STK25 | O00506.1 | STK3 | Q13188.1 |
| STN | Q9H668.1 | SUPT7L | O94864.1 | Survivin | O15392.1 | SUV39H1 | O43463.1 |
| SYCE1 | Q8N0S2.1 | SYCP1 | Q15431.1 | SYCP3 | Q8IZU3.1 | SYT | Q15532.1 |
| TA-4 | Q96RI8.1 | TACC1 | O75410.1 | TAF1B | Q53T94.1 | TAF4 | O00268.1 |
| TAF7L | Q5H9L4.1* | TAG-1 | Q02246.1* | TALI | P17542.1 | TAL2 | Q16559.1 |
| TAPBP | O15533.1 | TATI | P00995.1 | TAX1BP3 | O14907.1 | TBC1D3 | Q8IZP1.1 |
| TBP-1 | P17980.1 | TCL1A | P56279.1 | TCL1B | O95988.1 | TDHP | Q9BT92.1 |
| TDRD1 | Q9BXT4.1* | TDRD4 | Q9BXT8.1* | TDRD6 | O60522.1* | TEKT5 | Q96M29.1* |
| TEX101 | Q9B114.1* | TEX14 | Q8IWB6.1* | TEX15 | Q9BXT5.1* | TEX38 | Q6PEX7.1* |
| TF | P02787.1 | TFDP3 | Q5H9I0.1* | TFE3 | P19532.1 | TGFBR1 | P36897.1 |
| TGFBR2 | P37173.1 | THEG | Q9P2T0.1* | TIE2 | Q02763.1 | TIPRL | O75663.1 |
| TLR2 | O60603.1 | TMEFF1 | Q8IYR6.1* | TMEFF2 | Q9UIK5.1* | TMEM108 | Q6UXF1.1* |
| TMEM127 | O75204.1 | TMPRSS12 | Q86WS5.1* | TNC | P24821.1 | TNFRSF17 | Q02223.1 |
| TNFSF15 | O95150.1 | TNK2 | Q07912.1 | TOMM34 | Q15785.1 | TOP2A | P11388.1 |
| TOP2B | Q02880.1 | TOR3A | Q9H497.1 | TP73 | O15350.1 | TPA1 | 8N543.1 |
| TPGS2 | Q68CL5.1 | TPI1 | P60174.1 | TPL2 | P41279.1 | TPM4 | P67936.1 |
| TPO | P40225.1 | TPPP2 | P59282.1* | TPR | P12270.1 | TPTE | P56180.1* |
| TRAF5 | O00463.1 | TRAG-3 | Q915P2.1* | TRGC2 | P03986.1 | TRIM24 | O15164.1 |
| TRIM37 | O94972.1 | TRIM68 | Q6AZZ1.1 | TRPM8 | Q7Z2W7.1 | TSGA10 | Q9BZW7.1* |
| TSP50 | Q9UI38.1* | TSPAN6 | O43657.1 | TSPY1 | Q01534.1* | TSPY2 | A6NKD2.1* |
| TSPY3 | Q6B019.1* | TSPYL1 | Q9H0U9.1 | TSSK6 | Q9BXA6.1* | TTC23 | Q5W5X9.1 |
| TTK | P33981.1* | TULP2 | O00295.1* | TUSC2 | O75896.1 | TWEAK | O43508.1 |
| TXNIP | Q9H3M7.1 | TYMS | P04818.1 | TYR | P14679.1 | U2 snRNP B | P08579.1 |
| U2AF1 | Q01081.1 | UBD | O15205.1 | UBE2A | P49459.1 | UBE2C | O00762.1 |
| UBE2V1 | Q13404.1 | UBE4B | O95155.1 | UBR5 | O95071.1 | UBXD5 | Q5T124.1 |
| UFL1 | O94874.1 | URI1 | O94763.1 | URLC10 | Q17RY6.1 | UROC1 | Q96N76.1 |
| USP2 | O75604.1 | USP4 | Q13107.1 | VAV1 | P15498.1 | VCX3A | Q9NNX9.1 |
| VEGFR1 | P17948.1 | VEGFR2 | P35968.1 | VHL | P40337.1 | VIM | P08670.1 |
| VWA5A | O00534.1 | WHSC2 | Q9H3P2.1 | WISP1 | O95388.1 | WNK2 | Q9Y351.1 |
| WNT10B | O00744.1 | WNT3 | P56703.1 | WNT-5a | P41221.1 | WT1 | P19544.1 |
| WWP1 | Q9H0M0.1 | XAGE-1 | Q9HD64.1* | XAGE-2 | Q96GT9.1* | XAGE-3 | Q8WTP9.1* |
| XAGE-4 | Q8WWM0.1 | XAGE-5 | Q8WWM1.1* | XBP1 | P17861.1 | XPO1 | O14980.1 |
| XRCC3 | O43542.1 | YB-1 | P67809.1 | YEATS4 | O95619.1 | YES1 | P07947.1 |
| YKL-40 | P36222.1 | ZBTB7A | O95365.1 | ZBTB7C | A1YPR0.1 | ZEB1 | P37275.1 |
| ZFYVE19 | Q96K21.1 | ZNF165 | P49910.1* | ZNF185 | O15231.1 | ZNF217 | O75362.1 |
| ZNF320 | A2RRD8.1 | ZNF395 | Q9H8N7.1 | ZNF645 | Q8N7E2.1* | ZUBR1 | Q5T457.1 |
| ZW10 | O43264.1 | ZWINT | O95229.1 | Ropporin-1A | Q9HATO | WBP2NL | Q6ICG8.1 |

CTAs/TSAs = bold and*
Table 2 optionally excludes Ropporin-JA Q9HATO and/or WBP2NL Q6ICG8.1.

TABLE 3
LIST OF ACCESSION NUMBERS FOR VIRAL ANTIGENS FROM IEDB

| | Q76R62.1 | P03182.1 | P09258.1 | P09310.1 | P03227.1 | P89466.1 | P04601.1 |
|---|---|---|---|---|---|---|---|
| P13285.1 | P09991.1 | P03468.1 | A2T3Q0.1 | P0C6X7.1 | P89448.1 | P12978.1 | P09257.1 |
| P50641.1 | P14075.1 | 20178567.1 | Q01023.1 | P03188.1 | P04585.1 | P0C767.1 | P12977.1 |
| P89467.1 | Q9W850.1 | Q00683.1 | P04591.1 | P03211.1 | 9628706.1 | P03460.1 | P08666.1 |
| P03485.1 | Q04360.1 | Q913Y7.1 | P89449.1 | Q81871.1 | P03452.1 | P17763.1 | P89430.1 |
| P03410.1 | P04012.1 | P27958.1 | Q6WB99.1 | P25212.1 | Q9PZT1.1 | P68593.1 | P03203.1 |
| P29996.1 | 9629374.1 | P59633.1 | O42053.1 | P0C6L3.1 | P59635.1 | Q9YZN9.1 | Q6WB95.1 |
| P10233.1 | P89475.1 | Q6WB98.1 | Q6SW67.1 | Q7TFA0.1 | P0CK17.1 | P59594.1 | 1980491.1 |
| P14079.1 | P15423.1 | 1891762.1 | P09259.1 | P09269.1 | Q77Q38.1 | Q786F2.1 | Q6SW99.1 |
| P24771.1 | F5HB98.1 | 9629370.1 | P68336.1 | P03300.1 | 1980486.1 | Q69027.1 | P28284.1 |
| P13290.1 | 9626585.1 | P06923.1 | P14076.1 | P03346.1 | O42062.1 | P07566.1 | P03204.1 |
| Q69091.1 | P09255.1 | P03206.1 | O36634.1 | P10205.1 | F5HCM1.1 | P0CK16.1 | Q6WB97.1 |
| Q85601.1 | P89468.1 | Q69467.1 | P03218.1 | Q786F3.1 | P59637.1 | 1891763.1 | Q6WB94.1 |
| P03231.1 | Q9IK92.1 | Q6WBA1.1 | P03466.1 | P14335.1 | P26670.1 | Q9PZT0.1 | 1985356.1 |
| Q2HR63.1 | P59634.1 | Q6SW59.1 | P03277.1 | P59595.1 | Q69028.1 | P03383.1 | P03261.1 |
| P03200.1 | P04578.1 | P06484.1 | F5HC97.1 | S5TC82.1 | P18095.1 | Q96895.1 | P18094.1 |
| 9629372.1 | P50791.1 | P03230.1 | P13845.1 | 9629712.1 | P03209.1 | P03129.1 | Q76R61.1 |
| P03228.1 | P0C206.1 | Q9WMB5.1 | P03226.1 | Q9QR69.1 | O36633.1 | O42049.1 | P03496.1 |
| P03428.1 | P03431.1 | P0C0U1.1 | P03433.1 | P03508.1 | 1980456.1 | P0C739.1 | P69726.1 |
| P69723.1 | 1980490.1 | 532129755.1 | P03120.1 | P04020.1 | P06922.1 | P03114.1 | P03314.1 |
| P06790.1 | P06788.1 | P06927.1 | P03101.1 | P03107.1 | P06794.1 | 530787712.1 | P04013.1 |
| Q80872.1 | P04014.1 | P03126.1 | P36811.1 | P06463.1 | P26554.1 | P04016.1 | P14078.1 |
| P03191.1 | 1980471.1 | P06821.1 | P0C797.1 | F5HF49.1 | P0C045.1 | P04296.1 | P04485.1 |
| P10230.1 | P10221.1 | P06487.1 | P10215.1 | P04293.1 | P10211.1 | P10209.1 | P10225.1 |
| P10224.1 | P10238.1 | P10185.1 | P08392.1 | P10231.1 | P06492.1 | P04290.1 | P08393.1 |
| P08543.1 | P10210.1 | P08617.1 | F5HB53.1 | P04019.1 | P04015.1 | P89442.1 | P89452.1 |
| P89462.1 | P59632.1 | O36635.1 | P07210.1 | Q83884.1 | Q8JUX5.1 | P03089.1 | Q66479.1 |
| P03185.1 | P0CAP6.1 | P04618.1 | 56160929.1 | 1980519.1 | P08669.1 | P14348.1 | P03212.1 |
| P03179.1 | 45617- | 1511872.1 | 302317869.1 | P69899.1 | P09247.1 | Q05127.1 | P18272.1 |
| | other.1 | | | | | | |
| Q9YMG2.1 | Q05128.1 | 302371215.1 | 302371218.1 | Q5XX08.1 | 302371214.1 | P14336.1 | 138948- |
| | | | | | | | other.1 |
| P08292.1 | 1803956.1 | P35253.1 | 1891726.1 | P09308.1 | P03189.1 | 667489389.1 | P09272.1 |
| 34365530.1 | Q05320.1 | P59596.1 | P32886.1 | 55097.1 | P03316.1 | P03276.1 | Q81870.1 |
| Q81862.1 | 64320.1 | 1933190.1 | | | | | |

TABLE 4
LIST OF ACCESSION NUMBERS FOR BACTERIAL ANTIGENS FROM IEDB

| | | | | | | | |
|---|---|---|---|---|---|---|---|
| | B8ZUD1.1 | P09621.1 | P9WPE5.1 | Q2GI62.1 | P0A5B8.1 | O50443.1 | Q5NEZ3.1 |
| P9WQF5.1 | P9WK95.1 | O05311.1 | P9WQD7.1 | P9WKG3.1 | P9WHE5.1 | P0CD83.1 | P9WHB9.1 |
| P9WH91.1 | P9WHE3.1 | P9WNK7.1 | A0A0F3MKF3.1 | A1JIP3.1 | B2RKS6.1 | P0A1D3.1 | P0A6F5.1 |
| P0C0Z7.1 | P0C923.1 | P61439.1 | Q9Z708.1 | P0A521.1 | P9WPE7.1 | Q79FJ2.1 | B8ZR84.1 |
| I6Y3P5.1 | Q2FYP2.1 | P9WG41.1 | P96890.1 | O06625.1 | I6X654.1 | Q8YIE1.1 | P9WQ81.1 |
| I6XWA1.1 | P11311.1 | O53900.1 | P9WIR7.1 | P9WQB1.1 | B8ZUC6.1 | O06802.1 | P9WMK1.1 |
| P9WG37.1 | Q2FWC4.1 | Q2GGE3.1 | O33347.1 | P9WJ09.1 | P9WJ11.1 | P9WF23.1 | O69703.1 |
| I6X4K0.1 | B2RM93.1 | P71888.1 | P9WFW3.1 | P9WPV1.1 | P9WPU7.1 | P9WPV3.1 | P9WPU5.1 |
| 050391.1 | P9W1D7.1 | P9WPC3.1 | P96901.1 | O84848.1 | Q2FUX4.1 | A0A0M1YNY3.1 | P49944.1 |
| P9WPQ9.1 | Q45010.1 | Q2FZK7.1 | P9WMN3.1 | P9WPQ1.1 | Q45013.1 | O53666.1 | Q5NEH1.1 |
| P9WHR5.1 | P9WIE5.1 | Q5NEQ3.1 | P9WNF3.1 | F2QBN0.1 | B8ZTB7.1 | P0C922.1 | P9WMJ9.1 |
| Q5NGW2.1 | P01556.1 | Q8DMZ4.1 | P33768.1 | Q2FUY2.1 | Q5NG56.1 | X8CE55.1 | Q5NGE4.1 |
| P94973.1 | O06827.1 | P96872.1 | I6X9Y7.1 | I6XFZ8.1 | O50442.1 | O53697.1 | O53978.1 |
| P95137.1 | P95144.1 | O53519.1 | Q79FZ8.1 | P9WJF5.1 | P71629.1 | P9WJS3.1 | P9WPB7.1 |
| Q7D9T1.1 | P9WHS1.1 | O06393.1 | P9WP69.1 | P9WPN5.1 | P9WNX3.1 | O53380.1 | I6YAU3.1 |
| P0A4V2.1 | P9WQP3.1 | P0C2T2.1 | P9WQP1.1 | P9WQN9.1 | O53311.1 | P9WIS7.1 | O06159.1 |
| H2GU79.1 | Q2G2Q0.1 | P9WNV1.1 | P9WNV5.1 | Q8YE98.1 | Q59191.1 | P9WGY7.1 | P9WGY9.1 |
| Q2G2W1.1 | P9WGH1.1 | P9WNG9.1 | P9WNG7.1 | O84591.1 | Q9Z7A6.1 | P9WGR1.1 | P96404.1 |
| I6YGS0.1 | Q6MX18.1 | P9WNK5.1 | O53692.1 | P9WNK3.1 | P9WNK1.1 | P9WNJ9.1 | P9WNJ7.1 |
| P9WNJ5.1 | P9WNJ3.1 | P9WNJ1.1 | P9WNI9.1 | P96903.1 | P9WNB1.1 | P9WJE1.1 | P9WJD9.1 |
| P9WJD7.1 | P9WJD3.1 | P9WJC5.1 | P9WJC3.1 | P9WJC1.1 | P9WWQ3.1 | P9WJE5.1 | P9WJC7.1 |
| O84646.1 | I6YDV4.1 | P11439.1 | Q5NFJ1.1 | P9WNE5.1 | P14738.1 | P11089.1 | H7C7G3.1 |
| L7N6B9.1 | I6XFI7.1 | O05578.1 | P96218.1 | P9WN39.1 | P9WN59.1 | Q8YBI3.1 | P9WN83.1 |
| P9WJA9.1 | P9WMY9.1 | Q5NH51.1 | O53673.1 | P9WIP9.1 | P0CE15.1 | P72041.1 | Q5NEM8.1 |
| Q5NI16.1 | P9WJA3.1 | P0A4Q1.1 | P9WIP1.1 | P9WIN9.1 | P9WNF5.1 | O50846.1 | Q59947.1 |
| H7C7N8.1 | Q5NEC6.1 | O84606.1 | P9WQJ9.1 | P9WQJ7.1 | P9WQ71.1 | O53611.1 | P9WKL1.1 |
| P9WKJ7.1 | D5V9Y8.1 | P0CC04.1 | P23700.1 | P9WJN5.1 | Q5NHJ0.1 | Q5NEY9.1 | P15917.1 |
| Q2G155.1 | O34094.1 | Q8F8E1.1 | O69661.1 | H6MMU4.1 | P9WK61.1 | P9WK55.1 | Q8YGS9.1 |
| O50811.1 | P9WQ59.1 | P9WIN7.1 | P9WIR1.1 | O50430.1 | D5VCH6.1 | Q5NHI7.1 | P9WFU9.1 |
| I6XFY8.1 | B2RH54.1 | Q46409.1 | P30690.1 | A0A0J5IWN3.1 | A0PSI5.1 | A4TAC4.1 | B1MB69.1 |
| B2HSY2.1 | B8ZSN3.1 | E4WHS0.1 | P9WK17.1 | V5XE39.1 | I6X7G8.1 | I6Y461.1 | I6YGB1.1 |
| I6YC99.1 | Q79FY7.1 | I6X5Z8.1 | I6Y479.1 | I6YA32.1 | O05461.1 | Q2G1E2.1 | P9WK19.1 |
| I6YAW3.1 | Q5NGG4.1 | O51624.1 | P9WJW5.1 | Q50584.1 | B2RHG1.1 | Q5NFL7.1 | P9WQN7.1 |
| P9WHH3.1 | 084639.1 | Q5NF24.1 | P9WJH1.1 | P9WJH5.1 | O53203.1 | P55969.1 | O50418.1 |
| Q5NGE0.1 | H7C7K8.1 | O54584.1 | G1UB30.1 | Q5NH85.1 | G1UB25.1 | P0A3N8.1 | E1X6Y5.1 |
| Q5NEP7.1 | Q8YHH0.1 | P38006.1 | P43838.1 | P43839.1 | P0CL67.1 | P0CL66.1 | Q0SIZ0.1 |
| Q07337.1 | G51X16.1 | O07721.1 | 053254.1 | P75330.1 | I6Y936.1 | L7N649.1 | L7N656.1 |
| L7N693.1 | Q79FK4.1 | Q79FR3.1 | Q79FR5.1 | Q79G04.1 | Q79FS8.1 | Q6MWX1.1 | Q79FV6.1 |
| Q79FS5.1 | Q79FQ7.1 | Q79FP3.1 | Q79FP2.1 | Q79FK9.1 | Q79FE6.1 | I6XEF1.1 | Q79FD4.1 |
| Q6MX26.1 | Q6MX50.1 | L7N680.1 | O53695.1 | I6X8R2.1 | O53246.1 | I6Y0L1.1 | Q2G282.1 |
| P14283.1 | P04977.1 | P9WMX7.1 | P9WFR1.1 | P9WN09.1 | O86345.1 | P9WGU1.1 | P9WGT9.1 |
| P9WGT7.1 | P9WPF7.1 | P9WIB3.1 | P9WMM9.1 | P9WHM5.1 | P9WQE9.1 | Q8DQ08.1 | Q8DQ07.1 |
| I6Y231.1 | P9WHV9.1 | O05877.1 | O07236.1 | O86370.1 | O06404.1 | O06410.1 | B8ZRL2.1 |
| O06807.1 | 033269.1 | Q79FA9.1 | Q79FK6.1 | Q8VKN2.1 | L7N675.1 | Q79FK5.1 | L0T7Y7.1 |
| Q79F19.1 | Q79FE1.1 | Q6MWX9.1 | O84616.1 | O84647.1 | P9WQ27.1 | O84288.1 | I6X9S5.1 |
| P9WJW3.1 | P9WPS9.1 | P95149.1 | O53632.1 | I6Y293.1 | L0T243.1 | P9WP43.1 | P9WKC9.1 |
| P96402.1 | P71810.1 | O06417.1 | P96365.1 | L0T5B2.1 | P96264.1 | P9WJK5.1 | P9WJQ9.1 |
| O84419.1 | O84818.1 | Q8YG32.1 | O06608.1 | O07175.1 | P9WGA3.1 | O53323.1 | P96354.1 |
| P9WIM9.1 | B8ZRT2.1 | P9WK93.1 | P13423.1 | O84583.1 | P9WG63.1 | P9WIM1.1 | P9WKJ3.1 |
| P9WNZ7.1 | P9WK31.1 | Q50701.1 | P9WID3.1 | Q8YC41.1 | P9WPL3.1 | P9WNI3.1 | P9WNI7.1 |
| P9WNI5.1 | P9WQ49.1 | P9WMG1.1 | Q2GGR3.1 | P9WK71.1 | O33192.1 | P9WND5.1 | P9WFL9.1 |
| P9WMB7.1 | P9WJ79.1 | P9WND7.1 | Q63RA7.1 | Q63ID0.1 | I6YET7.1 | Q9S010.1 | P9WGC9.1 |
| Q50700.1 | Q5NFR6.1 | P9WGK3.1 | P9WHI1.1 | P9WHV3.1 | Q5NIA7.1 | P9WG27.1 | P9WF73.1 |
| P9WGA1.1 | P9WIB9.1 | P9WGL3.1 | O51381.1 | P9WI83.1 | P9WI79.1 | P9WFT7.1 | Q8YGS6.1 |
| P05788.1 | P17835.1 | P9WIK9.1 | Q5NHP7.1 | P9WJU5.1 | P9WGE7.1 | Q2G2B2.1 | P04958.1 |
| P9WG67.1 | P9WKE1.1 | O07226.1 | P9WJ13.1 | P9WHF3.1 | P9WF43.1 | Q7D7L0.1 | P9WMF9.1 |
| P9WGN1.1 | P9WKJ9.1 | P60230.1 | P9WKH7.1 | O53699.1 | P9WHT7.1 | P9WJS5.1 | Q5NII0.1 |
| Q8YDZ3.1 | Q9RPX7.1 | P9WN67.1 | O05576.1 | Q5NHL4.1 | P9WN15.1 | P9WMD5.1 | P9WMF5.1 |
| P9WG85.1 | P9WJW7.1 | P9WIH1.1 | P9WIG1.1 | P9WIG3.1 | P9WIF5.1 | P9WIF1.1 | P9WIE7.1 |
| P9WHW9.1 | P9WI41.1 | P9WI39.1 | P9WI37.1 | P9WI25.1 | Q11031.1 | P9WI47.1 | P9WI23.1 |
| P9WI19.1 | P9WI11.1 | P9WI45.1 | P9WI07.1 | P9WI05.1 | Q79FH3.1 | P9WI43.1 | P9WHZ7.1 |
| P9WHZ5.1 | P9WHZ3.1 | P9WHY9.1 | P9WHY7.1 | P9WHY5.1 | Q6MX07.1 | P9WHY3.1 | Q6MWY2.1 |
| Q50703.1 | P9WHX3.1 | P96221.1 | Q7D589.1 | P9WMA3.1 | P9WKW1.1 | P9WKS9.1 | P9WM29.1 |
| P9WGC1.1 | P9WLZ5.1 | P9WLZ3.1 | P9WLX1.1 | P9WLV9.1 | P9WLS7.1 | P9WLQ1.1 | P9WLJ1.1 |
| P9WLH9.1 | P9WLF3.1 | P9WL97.1 | P9WL87.1 | P9WL85.1 | P9WL83.1 | P9WL67.1 | P9WL63.1 |
| P9WL51.1 | P9WL47.1 | P9WNH3.1 | P9WGL7.1 | P9WQM5.1 | P9WPD9.1 | A0A098A1N7.1 | A0A098A2B0.1 |
| A2RGM0.1 | A5LVF6.1 | A5MKZ9.1 | B8ZQI8.1 | B8ZQM3.1 | B8ZQT5.1 | B8ZR82.1 | B8ZRH1.1 |
| B8ZS71.1 | B8ZS85.1 | B8ZS86.1 | B8ZSJ5.1 | B8ZSL3.1 | B8ZSL7.1 | B8ZSM6.1 | B8ZT30.1 |
| B8ZTD0.1 | B8ZTS2.1 | B8ZTV5.1 | B8ZU53.1 | B8ZUA4.1 | B8ZUE5.1 | B8ZUF0.1 | B8ZUT6.1 |
| B8ZUX6.1 | C0R9U8.1 | C6DPT8.1 | C6DQ35.1 | E1XJN6.1 | G8W6L3.1 | G8W6L7.1 | G8W6U7.1 |
| H6MNY3.1 | H6MQD5.1 | H8HRN0.1 | H8HW90.1 | H8L8K3.1 | I6TQ53.1 | I6TX52.1 | P005B9.1 |
| Q1BYS7.1 | R4MDK6.1 | S5F815.1 | W6GWM1.1 | P9WFC9.1 | P9WFJ9.1 | P14916.1 | P69996.1 |
| P9WFC5.1 | Q8VKQ6.1 | P9WHS3.1 | A5MKI6.1 | | | | |

TABLE 5
LIST OF ACCESSION NUMBERS FOR FUNGAL ANTIGENS FROM IEDB and UNIPROT

| | | | | | | | |
|---|---|---|---|---|---|---|---|
| Q5ANA3.1 | Q5A3P6.1 | Q59VM7.1 | Q5A1A9.1 | Q5APF0.1 | Q8J0P4.1 | Q4WHG0.1 | Q4WQ87.1 |
| Q59X67.1 | Q59Z17.1 | Q59ZI3.1 | Q5AA33.1 | B8N4Q9.1 | Q4WAW6.1 | Q4WAJ6.1 | Q4X1V0.1 |
| A0A1D8PQ86.1 | Q59ZB1.1 | Q873N2.1 | Q59L72.1 | B8NIF0.1 | P46075.1 | Q4WCL1.1 | Q4WRP2.1 |
| Q59L12.1 | Q59LC9.1 | P48989.1 | Q5AFC2.1 | B8N406.1 | Q4WGL5.1 | Q9HEQ8.1 | Q4WVI6.1 |
| P46593.1 | P82611.1 | Q5ADV5.1 | Q59SG9.1 | P41750.1 | O00092.1 | Q4WEN1.1 | Q4WCV3.1 |
| P0DJ06.1 | 094038.1 | Q59WD3.1 | Q59RQ0.1 | B8NM71.1 | Q4WLW8.1 | Q4WI37.1 | Q4WNI1.1 |
| P29717.1 | P46589.1 | Q59W04.1 | Q59RK9.1 | B8MYS6.1 | Q8X176.1 | Q4WZS1.1 | Q4WQH4.1 |
| Q9UW14.1 | Q5AF56.1 | Q59VN0.1 | P31353.1 | B8N8Q9.1 | Q96UX3.1 | Q4WDA4.1 | Q4WDE1.1 |
| Q92207.1 | P83773.1 | Q59WB9.1 | Q5ACM4.1 | B8N8R3.1 | Q4WPF5.1 | Q4WLS7.1 | Q4WJT7.1 |
| Q5A8T7.1 | Q59YU1.1 | Q59P53.1 | Q5ACI8.1 | B8N417.1 | Q92450.1 | Q4WWM6.1 | Q4WLG1.1 |
| Q5A8T4.1 | Q59YV2.1 | Q5A432.1 | Q5AB93.1 | B8N8R0.1 | Q4WAW9.1 | Q4WP81.1 | Q4WQR6.1 |
| P43076.1 | Q5ABE5.1 | Q5AK64.1 | Q5ALL8.1 | B8NM74.1 | A4GYZ0.1 | Q6MYT0.1 | Q4WZS2.1 |
| Q5AP53.1 | Q59LF2.1 | A0A1D8PNZ7.1 | Q5A4X8.1 | B8N106.1 | Q4WAW3.1 | Q4WIL0.1 | Q4MXP0.1 |
| Q5AL52.1 | Q8NJN3.1 | Q59Q30.1 | Q5AD34.1 | B8NHY4.1 | Q70J59.1 | Q4WXV2.1 | Q4WU59.1 |
| P43079.1 | Q5ALN1.1 | A0A1D8PN12.1 | Q59V02.1 | B8NJG8.1 | Q4X1A4.1 | Q4X0Z3.1 | Q4WUG4.1 |
| Q5AD07.1 | Q59S72.1 | Q5AK24.1 | Q5AHC0.1 | B8NM66.1 | E9R876.1 | Q4WN25.1 | Q4WIK9.1 |
| Q5A0E5.1 | Q59K86.1 | Q5AFT2.1 | Q59Y11.1 | B8MYL0.1 | M4VQY9.1 | Q4WN21.1 | Q4WYP0.1 |
| Q5AKU6.1 | Q5AGD1.1 | Q5A0W6.1 | Q59QA5.1 | B8NM62.1 | Q4WF53.1 | Q4X1N0.1 | Q4X0B5.1 |
| Q59RL7.1 | P79023.1 | P0CB63.1 | Q5AMJ5.1 | B8NGT5.1 | Q4WZ64.1 | Q4WQV2.1 | Q4WYK9.1 |
| G1UB61.1 | Q59LP6.1 | Q59U11.1 | Q5AMF7.1 | B8NM64.1 | Q4WAZ0.1 | Q4WZP2.1 | Q4WY33.1 |
| Q5ABC6.1 | Q5AP87.1 | P83775.1 | Q5ABW2.1 | B8NV37.1 | Q4WR16.1 | Q4WVK2.1 | Q4X1F8.1 |
| A0A1D8PQB9.1 | P22274.1 | Q5APF2.1 | Q5APJ9.1 | B8N151.1 | Q4WLB9.1 | Q4MUA0.1 | Q4WA45.1 |
| P87020.1 | Q5AC48.1 | Q59VP2.1 | Q5AM72.1 | B8NEJ3.1 | Q4WQS0.1 | A4DA84.1 | Q4WKD7.1 |
| P0CY27.1 | Q5AP59.1 | Q5AEE1.1 | Q5ACU3.1 | B8N8M2.1 | Q4WEP7.1 | Q4WJX0.1 | Q4WCH5.1 |
| Q59XX2.1 | Q59MV1.1 | Q5AMR5.1 | Q5A1V3.1 | B8MYV0.1 | E9R9Y3.1 | Q4WP38.1 | Q4WXY3.1 |
| Q59U10.1 | Q5AL27.1 | Q59SU5.1 | Q59RF7.1 | B8N7I7.1 | P41748.1 | Q4X1D7.1 | Q4WPL7.1 |
| Q59RW5.1 | Q5AJD2.1 | Q59VP1.1 | Q5ACN3.1 | B8NJG3.1 | Q4WYG3.1 | Q4W9Z9.1 | Q4X136.1 |
| Q59MQ0.1 | P0CU38.1 | Q5ADQ0.1 | Q5AHE8.1 | B8N8R1.1 | P87184.1 | Q4WE62.1 | Q4WZ44.1 |
| Q5ABU7.1 | Q59QC5.1 | Q5AK59.1 | Q5AHA4.1 | B8NJH2.1 | Q4WBS1.1 | Q4WZL3.1 | Q4WIC7.1 |
| Q9Y7F0.1 | Q5A5N6.1 | Q59RH5.1 | Q5AEG7.1 | B8NQ51.1 | Q70DX9.1 | Q4WB37.1 | Q4WMK2.1 |
| Q5AC08.1 | Q59Q79.1 | Q5ACW8.1 | Q59V01.1 | B8NM63.1 | Q4WG16.1 | Q4W9Z4.1 | Q4WNC9.1 |
| P30575.1 | Q5AH38.1 | Q5AGM0.1 | Q5AK97.1 | B8NM73.1 | Q96X30.1 | Q4WDD0.1 | Q4WY67.1 |
| Q5AAG6.1 | Q5AMN3.1 | Q59VN2.1 | Q5A1B2.1 | B8NYX0.1 | Q4WV19.1 | Q4WKB9.1 | Q4WU12.1 |
| O74189.1 | Q5A1Z5.1 | O94069.1 | Q5AJK6.1 | B8N3P7.1 | Q4WAZ6.1 | Q4WU07.1 | Q4WA61.1 |
| Q59W62.1 | Q5A6K2.1 | P0CY20.1 | Q59L96.1 | B8NJH1.1 | Q4W944.1 | Q4WBL6.1 | Q4WA58.1 |
| POCY34.1 | Q59L25.1 | Q59XQ1.1 | Q59MD0.1 | B8MXJ7.1 | Q4WTV7.1 | Q4WX13.1 | Q4WA60.1 |
| Q5A1D3.1 | Q5A922.1 | O94048.1 | Q5AG46.1 | B8NJB0.1 | Q4WMJ9.1 | Q4WV71.1 | Q4WX36.1 |
| Q5AJU7.1 | Q5AFG1.1 | Q5ADX2.1 | Q59VW6.1 | B8NP57.1 | Q4WZ65.1 | Q4X0C2.1 | Q4WA62.1 |
| Q5A4H5.1 | Q5ALR8.1 | P46586.1 | Q5A8I6.1 | B8N7Z8.1 | A0A067Z9B6.1 | Q4WRU4.1 | Q4WA59.1 |
| Q59Y31.1 | Q5AEI2.1 | P83776.1 | Q9UW24.1 | B8N5V5.1 | Q66WM4.1 | Q4WG54.1 | Q4WXQ7.1 |
| POCY29.1 | Q5AI71.1 | Q5A895.1 | Q59Q38.1 | B8MZA3.1 | Q6T267.1 | Q4WP13.1 | Q4WVA0.1 |
| Q5ANJ4.1 | Q5ABA6.1 | Q59PP0.1 | Q5ADLO.1 | B8NLY9.1 | Q4WLW5.1 | Q4WHG5.1 | Q4WDN4.1 |
| Q59NH8.1 | Q5ABX0.1 | Q5AHH4.1 | Q5AH11.1 | B8NR69.1 | Q4WMJ0.1 | Q4WPF7.1 | Q4WK03.1 |
| P0CY33.1 | Q5A4N0.1 | Q96UX5.1 | Q59W55.1 | B8MZ41.1 | Q4WQU0.1 | Q4WH83.1 | Q4WCG2.1 |
| Q00310.1 | Q59TN9.1 | P87206.1 | Q5AC37.1 | B8N757.1 | Q4WMJ8.1 | Q4WXW1.1 | Q4WX99.1 |
| Q5A0W9.1 | Q5A557.1 | Q5A029.1 | Q5A7Q3.1 | B8NR71.1 | Q4WWN8.1 | Q8NJM2.1 | Q4WV10.1 |
| Q5A4M8.1 | Q59UG3.1 | Q5A1E0.1 | Q59PV6.1 | A0A0D9MRV9.1 | Q4WZ63.1 | Q4WWD3.1 | Q4WIS6.1 |
| Q5AJC0.1 | P0C075.1 | Q59XL0.1 | P0CH96.1 | P55790.1 | Q4WVN4.1 | Q4WPU8.1 | Q4WP65.1 |
| Q595U1.1 | Q59R09.1 | Q5A6U1.1 | P83782.1 | B8NM72.1 | Q4WAY8.1 | Q4WN99.1 | Q4WUK1.1 |
| Q5AG71.1 | Q9B8D4.1 | Q5A8I8.1 | Q5A660.1 | B8MW78.1 | Q4WY07.1 | P0C959.1 | Q4WKN3.1 |
| Q5AMT2.1 | Q9B8D3.1 | Q59PR9.1 | Q59YT1.1 | Q9P900.1 | Q4WZ66.1 | Q4X057.1 | Q4WG58.1 |
| Q59KY8.1 | Q9B8D5.1 | O74261.1 | P53709.1 | B8NDE2.1 | Q4WQZ5.1 | Q4WPW2.1 | Q4WXX9.1 |
| Q59LY1.1 | Q59LR2.1 | Q96VB9.1 | Q5ACX1.1 | B8NJF4.1 | O42630.1 | Q4X1U0.1 | Q4WC37.1 |
| Q59UT4.1 | Q5AED9.1 | Q5AQ47.1 | Q5ADP9.1 | B8NIV9.1 | P0C7S9.1 | Q4WP57.1 | Q4X1Y0.1 |
| Q5ABC5.1 | Q5A4W8.1 | Q5A985.1 | Q92210.1 | B8NG16.1 | Q4WI46.1 | Q4WPH9.1 | Q4WZL8.1 |
| Q59MV9.1 | Q5ANH2.1 | Q59ZW2.1 | Q59MA3.1 | B8NX60.1 | Q4WQY4.1 | Q4WDK5.1 | Q4WR80.1 |
| Q59MD2.1 | Q5A649.1 | P83784.1 | Q5AFK3.1 | B8NM75.1 | Q4WAY3.1 | Q4WI71.1 | Q4WY53.1 |
| Q5A8N2.1 | Q5AI22.1 | Q59P11.1 | Q59563.1 | B8MZZ6.1 | Q4WT66.1 | Q4WY57.1 | Q4WL88.1 |
| P40953.1 | Q5A950.1 | Q5ADN8.1 | Q5A0Y2.1 | B8NM67.1 | Q6MY57.1 | Q4WY08.1 | Q4WGV9.1 |
| Q5APR8.1 | Q5ANC9.1 | Q5A849.1 | Q5ALW7.1 | B8NRX2.1 | P0C954.1 | Q4WND3.1 | Q4WC29.1 |
| P10613.1 | Q59UH7.1 | Q5A7R7.1 | Q59W52.1 | B8NXJ2.1 | Q4W946.1 | Q4X1D2.1 | Q4WKV8.1 |
| Q5A5Q6.1 | Q5ALX8.1 | Q59XBO.1 | Q59542.1 | B8NMD3.1 | Q4WMJ5.1 | Q6MY91.1 | Q4WYA5.1 |
| Q5A4F3.1 | Q5AI37.1 | Q59P96.1 | Q5A961.1 | B8NBI2.1 | Q7OGH4.1 | Q4WRV2.1 | Q4WCM6.1 |
| P43094.1 | Q5ABV4.1 | Q595R6.1 | Q595T6.1 | B8NPA4.1 | Q4WUL6.1 | Q4WRX4.1 | Q4WKB2.1 |
| Q9P940.1 | Q5AKU4.1 | Q9P975.1 | Q59N74.1 | B8N803.1 | P61832.1 | Q4WP03.1 | Q4WNG7.1 |
| Q5AJY5.1 | Q59VY1.1 | O94083.1 | Q5A6P6.1 | B8NPT0.1 | Q4WG11.1 | Q4WTA6.1 | Q4WRE8.1 |
| P39827.1 | Q59Z51.1 | Q5AIA4.1 | Q59XMO.1 | B8MXP5.1 | Q4WYU4.1 | Q4WZJO.1 | Q9P8P4.1 |
| Q59WF4.1 | Q59LV8.1 | Q59YF4.1 | Q5A4N5.1 | B8NIB8.1 | Q4WYR6.1 | Q4W958.1 | Q4WJ54.1 |
| P83774.1 | Q59X11.1 | Q59XW9.1 | Q5A6M2.1 | B8N9H4.1 | Q4WNE1.1 | Q4X054.1 | Q4WHW1.1 |
| Q59Q46.1 | Q5ABQ7.1 | Q59WU8.1 | Q5A5M7.1 | B8NNK9.1 | Q4WQZ6.1 | Q4X1I3.1 | Q4WYG7.1 |
| Q59X23.1 | Q59PZ3.1 | Q5AAR0.1 | Q5A6N8.1 | B8NI03.1 | Q4WWC6.1 | Q4W9V1.1 | Q4WJH4.1 |
| P46614.1 | O13332.1 | Q5AQ62.1 | Q9UVJ4.1 | B8NM76.1 | Q6Q487.1 | Q4WDF1.1 | Q4WJM6.1 |
| Q5AQ33.1 | Q5AHD6.1 | Q59R35.1 | Q59V88.1 | B8NM79.1 | P0C957.1 | Q4WWN2.1 | Q4WMB6.1 |
| P82610.1 | A0A1D8PPG4.1 | Q5A847.1 | Q59RA0.1 | B8NJG9.1 | Q4WM08.1 | Q4WTH0.1 | Q4WMU9.1 |
| Q5AP80.1 | Q5ADW3.1 | Q5A6A4.1 | Q59XU5.1 | B8NPL7.1 | Q4W9B8.1 | Q4WJQ1.1 | Q4WIF3.1 |
| P46598.1 | Q5AML6.1 | Q5A4Q1.1 | Q5AH12.1 | B8NMR5.1 | Q4WWJ1.1 | Q4WKL7.1 | Q4WEH7.1 |
| Q5A506.1 | Q5A846.1 | P0CY22.1 | Q59ZX3.1 | B8NP65.1 | E9RCR4.1 | Q4WX90.1 | Q4WT34.1 |
| Q5A599.1 | A0A1D8PPI5.1 | P42800.1 | Q5AB48.1 | B8N556.1 | Q4WM67.1 | Q4WG69.1 | Q4WT99.1 |
| Q59NP5.1 | P0CT51.1 | Q59KI4.1 | Q5A3Q0.1 | B8NJ86.1 | Q4WUN7.1 | Q4WM32.1 | Q4XON1.1 |
| Q5AHA0.1 | Q59MA6.1 | Q59JU3.1 | Q5A6M0.1 | P41747.1 | E9QRF2.1 | Q4WTI3.1 | Q4W5A8.1 |
| Q07730.1 | Q5ALW2.1 | P83777.1 | Q5AL29.1 | P41765.1 | Q4WK60.1 | Q4WHX4.1 | Q4WLD1.1 |
| Q5AD05.1 | Q5ABU8.1 | Q5A310.1 | Q59KG2.1 | B8N6V7.1 | Q4WZ61.1 | Q4WXE9.1 | Q4WMU5.1 |
| Q5AME2.1 | Q5AEC6.1 | Q59N80.1 | O42825.1 | B8NKE9.1 | Q4W945.1 | Q4X0X6.1 | O13410.1 |
| P41797.1 | Q5A4X0.1 | Q5AJ77.1 | O59931.1 | B8NGU6.1 | Q4WMA6.1 | Q4W8Z9.1 | Q4WG40.1 |
| P0CY24.1 | Q59LX9.1 | Q59ZV4.1 | Q5AM44.1 | B8NBP9.1 | Q4WN58.1 | Q4WEB4.1 | Q4WLD5.1 |
| Q5ACZ2.1 | Q59PE7.1 | Q59XA7.1 | Q59RP7.1 | B8N8R2.1 | Q4WDE9.1 | Q4WDH3.1 | Q4WLD4.1 |
| Q5ABE2.1 | Q5ACL9.1 | Q59L13.1 | Q5AK94.1 | B8NKI4.1 | Q4WUR1.1 | Q4X1N4.1 | Q4WLD2.1 |
| Q59M56.1 | Q5ABT8.1 | Q5AG97.1 | Q5AKB1.1 | B8NQQ7.1 | Q4WQ08.1 | Q4WMP0.1 | Q4WLC9.1 |
| Q5AK51.1 | Q5AMH3.1 | Q5AB15.1 | Q59VM4.1 | B8NJHO.1 | Q4WF61.1 | A4D9B6.1 | Q4WQ54.1 |
| Q59UT5.1 | Q5AFF0.1 | Q59566.1 | Q5A246.1 | B8NKB9.1 | Q7LKT3.1 | Q4WD45.1 | Q4WAZ8.1 |
| Q5AAF4.1 | Q5AJC1.1 | Q59KN8.1 | Q5AJ92.1 | B8NM78.1 | Q4WQZ3.1 | Q4WM95.1 | Q4X161.1 |
| G1UBC2.1 | Q59VP0.1 | Q5A8X9.1 | Q5A2V2.1 | B8NTP7.1 | Q4WAZ3.1 | Q4X0I8.1 | Q4WB00.1 |
| Q5ADT1.1 | Q5AGC7.1 | Q5AFP8.1 | Q5ABP8.1 | B8MWJ5.1 | Q4WNV0.1 | Q4WLV6.1 | Q4WQ14.1 |
| O59923.1 | Q5AQ12.1 | Q9P8W1.1 | Q5AAV3.1 | B8N7G5.1 | Q4WRZ5.1 | Q4W9R2.1 | Q4WP12.1 |
| Q5AL03.1 | Q59X94.1 | Q9P8W0.1 | Q595N0.1 | B8NER4.1 | Q4WPF2.1 | Q4WAW8.1 | Q4WCR3.1 |
| Q5A2Z7.1 | Q5AFX2.1 | Q9P4E7.1 | Q5ACU6.1 | B8NJH3.1 | Q8LFZ1.1 | Q4WM50.1 | Q4WAQ9.1 |
| Q59VH7.1 | Q5A1E3.1 | Q9P8V9.1 | Q9Y7C4.1 | B8NDL1.1 | Q4WB03.1 | Q4WAW5.1 | Q6MYX6.1 |
| Q59KZ1.1 | O43101.1 | Q5A7Q6.1 | Q9HFQ7.1 | B8NWY6.1 | P40292.1 | Q4AX0.1 | Q4WZJ6.1 |
| Q5A960.1 | Q59WU0.1 | Q5A6N1.1 | Q5A3J1.1 | B8NC58.1 | Q4WPN0.1 | Q4WTQ4.1 | Q4WP59.1 |
| Q5AFA2.1 | Q5A893.1 | Q5AI58.1 | P40910.1 | B8NIM4.1 | Q4X1D4.1 | Q4WJ80.1 | Q4WLC8.1 |
| Q5A5U4.1 | P43069.1 | Q9P4E5.1 | Q5AQ57.1 | B8NXI4.1 | Q4WBW4.1 | Q4WD43.1 | Q4WVM1.1 |
| Q5AQ36.1 | Q59LN9.1 | P0CH67.1 | Q5ACL4.1 | B8NJG5.1 | Q4X180.1 | Q4WD44.1 | Q4WLP9.1 |
| Q9URB4.1 | Q5AA40.1 | Q5A387.1 | Q5A449.1 | B8NYD8.1 | Q4WQZ4.1 | Q4WD46.1 | Q4WHD2.1 |
| Q5AL36.1 | Q59S45.1 | Q59NB8.1 | Q59S27.1 | B8NYX1.1 | Q4WZ69.1 | Q4WD48.1 | Q4W9T6.1 |
| P86029.1 | Q5AM60.1 | Q92209.1 | Q59VF9.1 | B8NX76.1 | Q4WFK4.1 | Q4WD42.1 | Q4WR79.1 |
| O13289.1 | Q5AD67.1 | Q5A7M3.1 | Q5A7S5.1 | B8NL00.1 | Q4MUE0.1 | Q4WAX1.1 | Q4WHF8.1 |
| P43063.1 | Q59LV5.1 | Q59QC4.1 | O42817.1 | B8NSP6.1 | Q4WHP6.1 | Q4WY16.1 | Q4WV23.1 |
| Q5A651.1 | Q5AG86.1 | Q59PT4.1 | Q5AJ85.1 | B8N2I5.1 | Q4WWC5.1 | Q4WMJ1.1 | Q4WYA1.1 |
| Q59YH3.1 | Q59ST8.1 | Q5AKW3.1 | Q59P44.1 | B8NP78.1 | Q4WTN9.1 | Q4WBL2.1 | Q6MY48.1 |
| P82612.1 | O93803.1 | Q9P4E8.1 | Q59KC4.1 | B8NE46.1 | Q4WR17.1 | Q4WIQ0.1 | Q9UUZ6.1 |
| P53705.1 | Q5AFT3.1 | Q9P4E6.1 | Q59XV0.1 | B8NMK3.1 | Q4WA15.1 | Q4WID9.1 | Q4WRH5.1 |
| Q5AMQ6.1 | Q5A519.1 | Q59MJ2.1 | Q5ABV6.1 | B8NG97.1 | Q4WZ11.1 | Q4WPG0.1 | Q4WEU2.1 |
| Q9Y7W4.1 | O74161.1 | Q59LL4.1 | Q59UH5.1 | B8N316.1 | E9RD40.1 | Q4WDM5.1 | Q8NKF4.1 |
| Q5A688.1 | Q59RN6.1 | Q59S43.1 | Q5A869.1 | B8NYW9.1 | A4DA85.1 | Q4WYV0.1 | Q9HGV0.1 |
| P25997.1 | Q5A3Z5.1 | Q59P87.1 | Q5AEF2.1 | B8NBJ4.1 | P54267.1 | Q4WEY4.1 | Q4X156.1 |
| Q5AHG6.1 | P31225.1 | Q5AEN6.1 | Q5A0W7.1 | B8N7E5.1 | P0C958.1 | Q4WN24.1 | Q4WXZ5.1 |
| Q8TGH6.1 | Q59QC6.1 | Q59LF9.1 | P83783.1 | B8NM69.1 | Q4WQZ7.1 | Q4WQ21.1 | Q4X1R1.1 |
| Q5ABD0.1 | Q9I589.1 | Q5ADX5.1 | Q59XP0.1 | B8N306.1 | Q9Y8D9.1 | Q4WNN2.1 | Q4WXX4.1 |
| Q5AL16.1 | Q5A8W9.1 | P83778.1 | Q5AP66.1 | B8N7Z0.1 | Q4WBR2.1 | Q4WAH2.1 | Q4WJE9.1 |
| Q59RR0.1 | Q5APG6.1 | Q5AG31.1 | Q5AGZ9.1 | B8NJB2.1 | Q4WL66.1 | Q4WT40.1 | P41746.1 |
| Q59KM8.1 | Q59YD9.1 | Q5AHZ7.1 | Q5AFE4.1 | B8NWE1.1 | Q4WVG8.1 | Q4WFT3.1 | Q4WDF7.1 |
| Q5A220.1 | Q5AEN1.1 | Q5ACU4.1 | Q59PE4.1 | B8NIM7.1 | Q4WZ68.1 | Q4WQM4.1 | Q4X0T4.1 |
| Q92206.1 | Q8X1E6.1 | Q59PD6.1 | Q59LF3.1 | B8NKA3.1 | Q8TGG8.1 | Q4X0W8.1 | Q4WNX1.1 |
| Q59Z29.1 | P56553.1 | Q5A940.1 | Q9P8E3.1 | B8NK45.1 | Q4X0A9.1 | Q873N1.1 | Q4WTM9.1 |
| Q5AK66.1 | Q59WI7.1 | Q59M70.1 | Q59S78.1 | B8NBX4.1 | Q4WHG1.1 | Q4WR23.1 | Q4WQM6.1 |
| P46273.1 | Q5ALY0.1 | Q5A917.1 | Q59L89.1 | B8NVK8.1 | Q4WVE3.1 | Q4WEI5.1 | Q4WV66.1 |
| Q5AFI4.1 | Q5A0A9.1 | Q5ANA8.1 | P46250.1 | B8NI10.1 | Q4X162.1 | Q4WE68.1 | Q4WKH9.1 |
| Q5ALV2.1 | Q5A884.1 | Q5A3M6.1 | Q5AQ76.1 | B8NSW2.1 | Q6A3P9.1 | Q4WR21.1 | Q4WI01.1 |
| Q5A312.1 | Q9B8D8.1 | Q59MC8.1 | Q5AI21.1 | B8NA06.1 | Q4WQI1.1 | Q4WTC4.1 | Q873W8.1 |
| Q5A3V6.1 | Q59PZ7.1 | Q5A3K2.1 | Q96W54.1 | B8NLL0.1 | O43102.1 | Q4WPV8.1 | Q4WPW8.1 |
| Q59TB2.1 | Q9B1P9.1 | Q5A644.1 | P0CU35.1 | B8NBB2.1 | Q7Z8P9.1 | Q4WYF1.1 | Q4X1W8.1 |
| Q59KI0.1 | Q59MA9.1 | Q59ZH9.1 | O94150.1 | B8NBM3.1 | Q4WAY4.1 | Q4WJM7.1 | Q4WV30.1 |
| Q5APU2.1 | Q5ACH7.1 | Q71U11.1 | Q5ADT9.1 | B8NA66.1 | Q4X1Q4.1 | Q4WHP5.1 | Q4WUG9.1 |
| O42766.1 | P008K9.1 | Q5AJF1.1 | Q5A0L9.1 | B8NUL8.1 | Q4WJ90.1 | Q4WHU1.1 | Q4WYF4.1 |
| Q5A446.1 | Q59MF9.1 | Q59YV0.1 | Q5ACV9.1 | B8N076.1 | Q4X117.1 | Q4WT68.1 | Q4WK80.1 |
| Q59UY7.1 | Q5AI44.1 | Q59S85.1 | Q5A1D5.1 | B8NVB7.1 | Q4WBU0.1 | Q4U3Y2.1 | Q4WGU1.1 |
| Q5A6T5.1 | Q5AL10.1 | Q59PP6.1 | Q5A744.1 | B8NBC2.1 | Q4X228.1 | Q4WSM6.1 | Q4WYK1.1 |
| G1UB63.1 | Q5AED6.1 | Q59X40.1 | Q5A455.1 | B8NJL4.1 | Q6MYX3.1 | Q4W9B9.1 | Q4WNC1.1 |
| Q59QC7.1 | Q5AGE5.1 | O94030.1 | Q5AAU3.1 | B8NR70.1 | Q4X084.1 | Q4WHB7.1 | Q4WQC5.1 |
| P34948.1 | Q59LQ5.1 | Q5AL63.1 | Q9C0L9.1 | B8NGP8.1 | Q4X251.1 | Q4WNA1.1 | Q4WJS7.1 |
| P46592.1 | P0C8L0.1 | Q5A0Y8.1 | Q5AFV3.1 | B8NXS9.1 | Q4WHZ9.1 | Q4WHH4.1 | Q4WHK3.1 |
| Q5A872.1 | Q5A301.1 | Q5A723.1 | Q5A360.1 | B8NDZ1.1 | Q4WLA7.1 | Q4WA21.1 | Q4X0M4.1 |
| Q59QW5.1 | Q59X26.1 | Q5A1A0.1 | Q5AI90.1 | B8NW70.1 | Q4WXH8.1 | Q4WCP8.1 | Q4WLI5.1 |
| Q59WH0.1 | Q5AML2.1 | Q5A4G2.1 | Q5AD73.1 | B8MW97.1 | Q4WAS9.1 | Q4WVH0.1 | Q4WP54.1 |
| Q5A1N6.1 | Q59W50.1 | Q5A970.1 | Q5AD77.1 | B8N9M2.1 | Q4WZ60.1 | Q4WUJ6.1 | Q4WNH8.1 |
| Q5AAJ8.1 | Q59ZG8.1 | Q59Y46.1 | P87219.1 | B8N195.1 | Q4WYG2.1 | Q4WWP1.1 | Q4WTT2.1 |
| Q5AG40.1 | Q59VC6.1 | Q5AHC2.1 | Q59QH6.1 | B8MYS5.1 | A4D9R3.1 | Q4WS57.1 | Q4WEL6.1 |
| Q59P39.1 | Q59ZY9.1 | Q59V93.1 | Q59PT6.1 | B8NNI2.1 | Q4WR20.1 | Q4WVD9.1 | Q4WI38.1 |
| Q5AJB1.1 | Q5AL13.1 | Q59SI5.1 | Q5A5N5.1 | B8NJZ7.1 | Q4WA22.1 | Q4WK77.1 | Q4WTT7.1 |
| Q59UP6.1 | Q59NY7.1 | Q59RR3.1 | Q5ADL4.1 | B8N6H2.1 | Q4WM60.1 | Q4WCL2.1 | Q4WWS3.1 |
| Q5AMH6.1 | Q5AP89.1 | Q5APQ8.1 | Q5AM84.1 | B8NIX4.1 | Q0H904.1 | Q4WN75.1 | Q4WVH3.1 |
| Q59SF7.1 | Q59XY0.1 | P87207.1 | Q5AK73.1 | B8NGC8.1 | P78574.1 | Q4WES5.1 | Q4WD95.1 |
| Q59VX8.1 | Q5ADL9.1 | Q59MZ9.1 | Q5A4H9.1 | B8N970.1 | Q4WAR8.1 | Q4WVT3.1 | Q4WLP1.1 |
| Q59WG7.1 | P53698.1 | Q59Y41.1 | Q5ALX5.1 | B8MY73.1 | Q4WNK7.1 | Q4WVV6.1 | Q4WQI6.1 |
| Q5AFN8.1 | Q5AJX2.1 | Q59S52.1 | Q5A748.1 | B8N6W5.1 | P78746.1 | Q4WP83.1 | Q4WCJ7.1 |
| Q59TP1.1 | Q5APS5.1 | Q59U73.1 | Q5ALU2.1 | B8N3L3.1 | Q4WPF8.1 | Q4WAY7.1 | Q4WTT8.1 |
| Q5AF39.1 | Q59PG6.1 | Q9Y872.1 | Q5A2B9.1 | B8NPS8.1 | Q4WX43.1 | Q4WX89.1 | Q4WWR2.1 |
| Q5AP97.1 | Q59NP1.1 | Q5AGA9.1 | Q5ALX3.1 | B8NTI4.1 | Q4WQL4.1 | Q4WYT0.1 | Q4WWL0.1 |
| Q5A5U9.1 | Q59PD3.1 | Q59VL7.1 | Q5A1M3.1 | B8MYS7.1 | Q4WBE1.1 | Q4WNT9.1 | Q4WZT9.1 |
| Q5AF41.1 | Q5ACW2.1 | Q59KJ7.1 | Q5A4H4.1 | B8NM70.1 | Q4WQT2.1 | Q4WVS4.1 | Q4X0I7.1 |
| O13318.1 | Q5ANB2.1 | Q5AP90.1 | Q5AA26.1 | B8MYS8.1 | Q4WBT5.1 | A4D9J5.1 | Q4WU00.1 |
| Q5AA09.1 | Q5AJDO.1 | Q5AD72.1 | Q5ANL6.1 | B8N6M6.1 | Q4WQZ2.1 | Q4W9B7.1 | Q4WRW0.1 |
| Q5A762.1 | Q5A4P9.1 | Q59S59.1 | P87218.1 | B8NCU7.1 | Q4WD47.1 | Q4WNC6.1 | Q4W9V0.1 |
| P46587.1 | P78599.1 | Q5APM7.1 | Q59KF3.1 | B8N5T6.1 | Q4WCZ8.1 | Q4WJW8.1 | Q4WYJ7.1 |
| Q5A287.1 | Q5APC8.1 | Q5A2Z1.1 | Q59N29.1 | B8MVS3.1 | Q4WB01.1 | Q4WH96.1 | Q4WHY5.1 |
| Q59X49.1 | Q59LU0.1 | Q59TD3.1 | Q5A0L7.1 | B8NCM8.1 | Q4WBK6.1 | Q4X0I5.1 | Q4WEP0.1 |
| Q5ADM9.1 | Q5APT8.1 | P84149.1 | Q59UG4.1 | B8NW36.1 | Q4WRQ7.1 | Q4WMS9.1 | Q4WXD3.1 |
| Q5AH02.1 | Q59PR3.1 | Q5AI97.1 | Q5AHK2.1 | B8NJG7.1 | Q4WTQ6.1 | Q4WAH4.1 | Q4WJ02.1 |
| Q5A4X5.1 | Q5A2W2.1 | Q5A2A2.1 | Q5ADP6.1 | B8N7Z6.1 | Q4WJ21.1 | Q4WII4.1 | Q4WP96.1 |
| Q5A4E3.1 | Q5A4E2.1 | Q5A044.1 | Q5AK62.1 | B8NGU1.1 | Q4WPQ8.1 | Q4WJA1.1 | Q4WN54.1 |
| Q5A761.1 | Q5A309.1 | Q59P03.1 | Q59YF0.1 | B8NC10.1 | Q4WR62.1 | Q4W9R7.1 | Q4WCW2.1 |
| Q9UW23.1 | A0Q1D8PL26.1 | Q59TU0.1 | Q5AAJ7.1 | B8N4P0.1 | Q4WD56.1 | Q4WPP2.1 | Q4WPM6.1 |
| P53704.1 | P0CU37.1 | Q5APK7.1 | Q5A8H7.1 | B8NPN0.1 | Q4WIN6.1 | Q4WNQ6.1 | Q4WNW3.1 |
| Q59VR1.1 | Q5AF95.1 | Q5APK7.1 | Q69U81.1 | B8NQ08.1 | Q4U3E8.1 | Q4WNI0.1 | Q4WSI0.1 |
| G1UB67.1 | Q59MW2.1 | Q5A7N3.1 | Q5APB6.1 | B8N3N5.1 | Q4X195.1 | Q4WDG1.1 | Q4WNY4.1 |
| P52496.1 | Q59550.1 | Q5ANP2.1 | Q59WD5.1 | Q00049.1 | P0C955.1 | Q4X0Z7.1 | Q4WVF4.1 |
| Q9HEW1.1 | Q5AD78.1 | O59933.1 | Q5ABA2.1 | B8NDP1.1 | Q4WRH9.1 | Q4WM53.1 | Q4WP02.1 |
| Q5A6B6.1 | Q5AMM4.1 | Q3MPQ4.1 | Q5A861.1 | B8NEM4.1 | Q4WVD1.1 | Q4WN42.1 | Q4WWH6.1 |
| Q5A1W9.1 | Q5AAW3.1 | Q59MP1.1 | Q5AH87.1 | Q9P8Z9.1 | Q4WID6.1 | Q4WJH6.1 | Q4WVE5.1 |
| P30418.1 | Q59MG1.1 | Q59MB6.1 | P33181.1 | B8MZJ8.1 | Q4WFX9.1 | Q4WY51.1 | Q4WHP3.1 |
| Q595N6.1 | Q5ACK7.1 | Q5A216.1 | Q59Q43.1 | B8NX10.1 | Q4WRE4.1 | Q4WJ01.1 | Q4WRE2.1 |
| Q5A343.1 | Q5A218.1 | Q9UVL1.1 | Q5A860.1 | B8NV05.1 | Q4WC60.1 | Q4WGL2.1 | Q4WYX0.1 |
| Q5ABZ2.1 | Q595J9.1 | Q59Y57.1 | Q59ZW9.1 | B8NEI6.1 | Q4WR18.1 | Q4WP49.1 | Q4WRB8.1 |
| Q59MJ1.1 | Q5AD49.1 | Q5AGA0.1 | A0A1D8PI78.1 | B8MZI5.1 | Q4WQY6.1 | Q4WPE6.1 | Q4WI88.1 |
| Q5AJ71.1 | Q59NX9.1 | Q5A687.1 | Q59R24.1 | B8N5J0.1 | Q4WXK4.1 | Q4WWW9.1 | Q4WQL0.1 |
| O74201.1 | Q5A119.1 | Q59R28.1 | Q5AHJ5.1 | B8NDR8.1 | Q4WI96.1 | Q4WKB5.1 | Q4WDZ0.1 |
| Q5AK54.1 | Q59K07.1 | Q5AJ56.1 | P000X3.1 | B8NDQ2.1 | Q4WVH4.1 | Q4WA38.1 | Q4WA70.1 |
| O93852.1 | Q5AKA5.1 | Q5AD59.1 | Q59KL6.1 | B8N9M0.1 | A4D9R2.1 | Q4WHL1.1 | Q4WQ82.1 |
| Q5AIR7.1 | Q59QC2.1 | Q5AG73.1 | P43072.1 | B8NLN6.1 | P0C956.1 | Q4X1X0.1 | Q4WMX7.1 |
| Q5A8K2.1 | Q5AL45.1 | Q5AND1.1 | Q5AF54.1 | B8N9X2.1 | Q4WR22.1 | Q4WRX2.1 | Q4X0V2.1 |
| Q8TGB2.1 | P0CY19.1 | Q59NG5.1 | Q59W44.1 | B8NM08.1 | Q4WQY8.1 | Q4WDH9.1 | Q4WI16.1 |
| Q5A477.1 | Q5AGC4.1 | Q59N20.1 | P48990.1 | B8N5D4.1 | Q4WJJ3.1 | Q4WMG1.1 | Q4WXA1.1 |
| Q5AP95.1 | Q5ALP1.1 | Q59WJ5.1 | Q59U67.1 | B8N122.1 | Q4X265.1 | Q4WDE0.1 | Q4WCV5.1 |
| Q5AF03.1 | Q5AK42.1 | Q5AA50.1 | Q5ANB7.1 | B8NCF0.1 | Q9UVX3.1 | Q4WCX4.1 | Q4W9M7.1 |
| Q5AMQ4.1 | Q5APG7.1 | Q5A319.1 | Q5A3Y5.1 | B8NK51.1 | Q4WR19.1 | Q4X122.1 | Q4WQY9.1 |
| Q5ANI6.1 | Q59Y20.1 | Q5AD27.1 | Q59512.1 | B8N3R8.1 | Q4WIF3.1 | Q4WZF1.1 | Q4WX30.1 |
| P78595.1 | Q5ALL3.1 | Q5AHI7.1 | Q5APA2.1 | B8NG55.1 | Q4WLY1.1 | Q4WMU1.1 | Q4WUT7.1 |
| Q874I4.1 | Q5AAT0.1 | Q5ANE3.1 | P12461.1 | B8N0Q7.1 | Q4WMU3.1 | Q4WGB7.1 | Q4WIQ2.1 |
| Q9UWF6.1 | Q59QD6.1 | Q59506.1 | Q59TN1.1 | B8N513.1 | Q4WQG5.1 | A4DA73.1 | Q4X022.1 |
| Q9UW12.1 | Q5AML1.1 | P87185.1 | Q5A416.1 | B8N4F5.1 | Q4WPE9.1 | Q4WD81.1 | Q4WQZ0.1 |
| Q5AAL9.1 | Q5ACM9.1 | Q5AM50.1 | O43133.1 | B8NT06.1 | Q4WAZ4.1 | Q4WHG0.1 | Q4WE58.1 |
| Q5AD56.1 | Q59Z14.1 | Q9B8C8.1 | Q59MI8.1 | B8NHF2.1 | Q4WLN7.1 | Q4WAJ6.1 | Q4WJR4.1 |
| Q5A757.1 | Q5AAG1.1 | Q9B8C9.1 | Q5A302.1 | B8MWR8.1 | Q4WRB0.1 | Q4WCL1.1 | Q4WQZ1.1 |
| P28870.1 | Q59YL9.1 | Q9B8D2.1 | Q5AH60.1 | B8N4G0.1 | Q4WC55.1 | Q9HEQ8.1 | Q4WQY7.1 |
| Q59NX5.1 | Q59PL9.1 | Q9B8D1.1 | Q5A692.1 | B8N9M5.1 | Q4WMV5.1 | Q4WEN1.1 | Q4WQY5.1 |
| Q5ABG1.1 | Q59QL0.1 | Q59M69.1 | Q59Q39.1 | Q00278.1 | Q4WAZ2.1 | Q4WI37.1 | Q4WXT2.1 |
| Q5AP52.1 | Q5A1U8.1 | Q59VX9.1 | Q59NW5.1 | B8NPX1.1 | Q92197.1 | Q4WZ51.1 | Q8J130.1 |
| P0CY31.1 | O74198.1 | Q59YD8.1 | Q5A6Q4.1 | B8NYW8.1 | Q4W5E8.1 | Q4WDA4.1 | Q4WJX5.1 |
| P13649.1 | Q5A013.1 | Q59QH0.1 | P43075.1 | B8N219.1 | Q4WX94.1 | Q4WL57.1 | Q4X118.1 |
| Q5AG77.1 | P87163.1 | Q5A8A2.1 | Q59Q36.1 | B8NQK0.1 | Q4WLD0.1 | Q4WWM6.1 | Q4WVW4.1 |
| Q9UW13.1 | Q5AI86.1 | Q9B8D7.1 | Q92410.1 | Q12732.1 | Q4WUK5.1 | Q4WP81.1 | Q4WTH1.1 |
| P0CU34.1 | Q5AM80.1 | Q9UW25.1 | Q5A1M4.1 | Q9HEY7.1 | Q8IGG5.1 | Q6MYT0.1 | Q4WLI9.1 |
| P40954.1 | Q5A6Q7.1 | Q59XY9.1 | Q5ANC8.1 | Q6UEG8.1 | Q4WIK9.1 | Q4WIL0.1 | Q4WQJ5.1 |
| Q04802.1 | Q5AGV4.1 | Q5A2T0.1 | Q5A4K7.1 | O42716.1 | Q4WVU5.1 | Q4WXV2.1 | Q4WQJ2.1 |
| P0CY35.1 | Q5AJ82.1 | Q5AGW8.1 | Q5ADL8.1 | Q9UW95.1 | Q4WLM7.1 | Q4X0Z3.1 | Q4WK56.1 |
| Q5AAU5.1 | Q5AIA1.1 | Q5AD53.1 | Q59RQ2.1 | Q9Y8D9.1 | Q4W9P4.1 | Q4WN25.1 | Q4WJ52.1 |
| Q59VQ8.1 | Q5A9Z6.1 | Q5ACR4.1 | Q5APC0.1 | A2SZW8.1 | Q4WIT0.1 | Q4WN21.1 | Q4WJT9.1 |
| Q59VF4.1 | Q5AGC1.1 | P0CU36.1 | Q5A931.1 | Q2U2U3.1 | Q4WQB9.1 | Q4X1N0.1 | Q4WUV8.1 |
| Q5A0X8.1 | Q59ZV5.1 | Q5A2Y7.1 | Q59VW7.1 | Q00258.1 | Q4WGK6.1 | Q4WQV2.1 | Q4WX68.1 |
| O13426.1 | Q59VP7.1 | Q5A368.1 | Q5AKU5.1 | Q12437.1 | Q4WMR0.1 | Q4WZP2.1 | Q4WHN8.1 |
| Q5A0M4.1 | Q5A7P3.1 | Q9B8D6.1 | Q59MN0.1 | E9QYP0.1 | Q4WYE5.1 | Q4WVK2.1 | Q4WJU8.1 |
| Q59PF9.1 | Q5A6K8.1 | Q9B8D0.1 | Q59WH7.1 | Q4W576.1 | Q4WZ01.1 | Q4MUA0.1 | Q4WBT4.1 |
| Q5AFP3.1 | Q5AD13.1 | Q5A2K0.1 | Q96WL3.1 | Q4WMJ7.1 | Q4W930.1 | A4DA84.1 | Q4WZV6.1 |
| Q5AEK8.1 | Q04782.1 | Q5A1Q5.1 | Q59ZX6.1 | P28296.1 | Q4WBR0.1 | Q4WJX0.1 | Q4WUV9.1 |
| Q5AFK0.1 | Q5A0J9.1 | Q5AEM5.1 | Q59MU1.1 | E9RAH5.1 | Q4WHD1.1 | Q4WP38.1 | Q4WLV2.1 |
| Q5APD4.1 | Q59ZZ6.1 | Q5AK25.1 | Q5A0J0.1 | Q4WW81.1 | Q4WTB3.1 | Q4X1D7.1 | Q4WF52.1 |
| Q5ADQ9.1 | Q5AH25.1 | Q5AK10.1 | Q59WK2.1 | Q50EL0.1 | Q4WRV9.1 | Q4W9Z9.1 | Q4WBM1.1 |
| P83779.1 | Q59XM1.1 | Q5AI15.1 | P43073.1 | Q4WY82.1 | Q4X267.1 | Q4WE62.1 | Q4WAU7.1 |
| Q5AAH2.1 | Q59NN8.1 | Q5AEM8.1 | P87220.1 | Q4W5F6.1 | Q4WVZ3.1 | Q4WZL3.1 | Q4WZ53.1 |
| O74254.1 | Q5AP65.1 | Q5A4J4.1 | Q5ABD9.1 | E9RCK4.1 | Q4WR24.1 | Q4WB37.1 | Q4WPU9.1 |
| Q5AL49.1 | Q5AFF7.1 | Q59YK4.1 | P83781.1 | Q4WZA8.1 | Q4WPM8.1 | Q4W9Z4.1 | Q4WVZ0.1 |
| P53697.1 | Q59VR3.1 | Q59WV0.1 | Q5ANB1.1 | Q4WAW7.1 | Q4WE86.1 | Q4WDD0.1 | Q4WCX9.1 |
| Q5ACL7.1 | Q5AFH3.1 | Q5AHB1.1 | Q5A0E2.1 | Q92405.1 | A4DA70.1 | Q4WKB9.1 | Q4WJ38.1 |
| Q5AEM6.1 | P83780.1 | Q5APK0.1 | Q5AMG5.1 | Q4WRY5.1 | Q4WW45.1 | Q4WU07.1 | Q4WRC2.1 |
| Q8TG40.1 | Q5A4G9.1 | Q59PW0.1 | Q5A6T8.1 | Q7Z7W6.1 | Q4WVG2.1 | Q4WBL6.1 | Q4WWW5.1 |
| Q59X38.1 | Q59NQ9.1 | O74711.1 | Q59WG5.1 | Q4WZ67.1 | Q4WQG9.1 | Q4WX13.1 | Q4WC84.1 |
| Q59VQ3.1 | A0A1D8PNP3.1 | Q5ADN9.1 | Q5AI80.1 | Q4WZB3.1 | Q4WQN1.1 | Q4WV71.1 | Q4WIW3.1 |
| Q5A7Q2.1 | Q5A9Z1.1 | Q5ACP5.1 | Q5AB49.1 | Q4WLN1.1 | Q4WCF1.1 | Q4X0C2.1 | Q4WFV6.1 |
| Q5AJV5.1 | A0A1D8PK89.1 | Q5A1E1.1 | Q59R32.1 | Q4WR82.1 | Q4WZC3.1 | Q4WRU4.1 | Q4WKD9.1 |
| Q5A3Z6.1 | Q59WB3.1 | Q59L86.1 | Q5A061.1 | O14434.1 | Q4WYX7.1 | Q4WRU4.1 | Q4WP10.1 |
| Q5A201.1 | Q59ZC8.2 | Q5AD23.1 | Q59P50.1 | Q4WMK0.1 | Q4X0A5.1 | Q4WP13.1 | C5JZM2.1 |
| O93827.1 | Q5A1L6.1 | Q5A5U6.1 | Q59WC6.1 | Q4WPX2.1 | Q4WUD3.1 | Q4WHG5.1 | P0DJ06.1 |
| Q5AAI8.1 | A0A1D8PN14.1 | Q5ADQ7.1 | Q5AI48.1 | O43099.1 | Q4WS49.1 | Q4WPF7.1 | P46598.1 |
| Q5A2J7.1 | Q5A8X7.1 | Q59WJ4.1 | Q59ZU1.1 | Q4WJ81.1 | Q4WWCX7.1 | Q4WH83.1 | P87020.1 |
| P22011.1 | Q59X39.1 | Q5AGV7.1 | Q5AG56.1 | P67875.1 | Q4WXX5.1 | Q4WXW1.1 | P38110.1 |
| Q9HGT6.1 | Q5ACW6.1 | Q59NR8.1 | Q59T36.1 | Q4WZB4.1 | Q4WNB5.1 | Q8NJM2.1 | C1GK29.1 |
| Q9UW26.1 | P0CB54.1 | Q5A5K7.1 | Q9P840.1 | E9QUT3.1 | O42799.1 | Q4WWD3.1 | |
| Q59LX5.1 | A0A1D8PN88.1 | Q5A210.1 | Q5AHB8.1 | Q4WAZ9.1 | Q4WHA3.1 | Q4WPU8.1 | |
| Q59PT0.1 | A0A1D8PMB1.1 | Q59N10.1 | Q5AKU3.1 | Q4WZ70.1 | Q4W9M3.1 | Q4WN99.1 | |
| Q3MNT0.1 | Q5ABR2.1 | Q5A1B3.1 | Q59ZW4.1 | E9RBR0.1 | Q4WVH5.1 | P0C959.1 | |

### Model Population

The method may comprise the step of selecting or defining a model human population. A suitable model population is one that is relevant to the human population or a subpopulation in which it is intended to use the peptides designed or prepared by the method to induce a T cell response. This may be referred to as the target population or the intent-to-treat population. The peptides or the encoded peptides designed or produced by the method are for use in a method of inducing a T cell response against the target polypeptide in a subject of the intent-to-treat population. A relevant population is one that is representative or similar to the intent-to-treat population. In some cases the model population is representative for the whole human race. In other cases the model population may be a disease- and/or subject-matched population (subpopulation), for example a subpopulation matched to the intent-to-treat population by ethnicity, geographical location, gender, age, disease or cancer, disease or cancer type or stage, genotype, and/or expression of one or more biomarkers (for example, women having the BRCA mutation for a breast cancer vaccine), and/or partially by HLA genotype (for example subjects have one or more particular HLA alleles). In some cases the intent-to-treat population may be subjects having cancer or a type of cancer, such as any described herein. For example, the model population may have HLA class I and/or class II genomes that are representative of those found in the world population, or a subject and/or disease matched subpopulation. In some cases the model population is representative for at least 70%, or 75% or 80% or 84% or 85% or 86% or 90% or 95% of the intent-to-treat population by HLA diversity and/or HLA frequency. In some cases the model population may comprise at least 100, or 200 or 300 or 400 or 500 or 1000 or 5000 or 10000 or 15000 subjects.

Each subject in the model population is minimally defined by their HLA class I or class II genotype, e.g. complete 4-digit HLA class I genotype. Data concerning the HLA genotype of the model population may be stored or recorded in or retrieved from a database or be an in silico model human population.

### HLA-Binding Criteria

The method comprises the step of identifying, for each subject of the model population, amino acid sequences within the target polypeptide that meet certain HLA-binding criteria, such as comprising a T cell epitope that can bind to multiple HLA class I and/or class II HLA molecules as described herein. For example, amino acid sequences that comprise a T cell epitope that is capable of binding to at least three HLA class I alleles of a subject and/or a T cell epitope that is capable of binding to at least three or four HLA class II alleles of the subject are optimal for inducing CD4+ T cell and/or CD8+ T cell responses. In some cases the HLA class I-binding T cell epitope and the HLC class II binding T cell epitope may overlap. In some cases the HLA class I binding T cell epitope may be fully embedded in the sequence of the HLA class II binding T cell epitope. In some cases the multiple HLA class I and class II binding epitopes are within a minimum distance on one another, such as both within a 50, or 45, or 40, or 35, or 30, or 25 amino acid fragment of the target polypeptide.

The method comprises selecting a polypeptide fragment window length. The polypeptide fragment window length defines the fragment length across the target polypeptide used to identify hotspots where the maximum number of subjects in the model population have an amino acid sequence that meets the HLA-binding criteria. The polypeptide fragment window length may be from 9 to 50 amino acids long.

Peptides that comprise a hotspot sequence as identified by the method described herein may be particularly useful for inducing T cell responses in a high proportion of the subjects of the intent-to-treat population population. Peptides comprising such sequences may accordingly be designed or prepared according to the present disclosure and used in methods of treatment. The peptide may consist of the amino acid sequence of the hotspot fragment of the target polypeptide or may comprise the sequence of a longer fragment of the target polypeptide of which the hotpot sequence is a part. In some cases the target polypeptide fragment may be flanked at the N and/or C terminus of the peptide by additional amino acids that are not part of the consecutive sequence of the target polypeptide antigen. In some cases the fragment may be flanked by up to 30 or 25 or 20 or 15 or 10, or 9 or 8 or 7 or 6 or 5 or 4 or 3 or 2 or 1 additional amino acid at the N and/or C terminus.

In some cases the method of the disclosure may be repeated in an iterative process to identify further fragments of the target polypeptide antigen that meet the HLA-binding criteria in subjects of the model population. In some case the method may be repeated in up to 50, 45, 40, 35, 30, 25, 20, 19, 18, 17, 16, 15, 14, 13, 12, 11, 10, 9, 8, 7, 6, 5, 4, 3, 2 or 1 cycles of the method described herein.

In some cases, the object of the iterative process may be to identify the minimum number of peptides or hotspots that will induce the desired T cell responses (cytotoxic T cell response and/or helper T cell response) in the maximum number of subjects in the model or intent-to-treat population. In this case it is desirable to remove from the model population those subjects for whom the hotspots or peptides selected in any previous rounds already meet the desired criteria before repeating the method in a further cycle. The iterative method may in some cases be continued until either no more sequences meeting the HLA-binding criteria can be identified or a pre-defined number of cycles, number of hotspots, or pre-defined minimal coverage of the model or intent-to-treat population is reached.

In some cases further predefined criteria may be applied to the hotspot selection process. If a particular hotspot sequence does not meet such additional criteria then the hotspot may be disregarded and another amino acid sequence of the selected window length and meeting the HLA-binding criteria for the next highest number of subjects in the model population may be selected, until a sequence is reached that meets the additional predefined criteria. In an iterative process, the subjects of the model population for which the selected sequence meets all of the HLA-binding criteria and other criteria should be removed from the model population before proceeding to the next cycle.

In one example, the additional predefined criteria may relate to features of the peptide sequence that influence manufacturing feasibility. For example, in some cases a peptide/hotspot sequence may be rejected in it comprises a particular amino acid residue, such as a cysteine, or a particular amino acid motif, or if the peptide/hotspot sequence has less than a minimum level of hydrophilicity.

The method of the disclosure may be used to provide peptides that are useful for inducing T cell responses against a given polypeptide, or to provide an ideal set of peptides from which to select a peptide for inducing T cell responses against one or more given polypeptides in a specific subject of a given human population.

In other cases the method may be repeated for a set of polypeptides, for example a set of polypeptides that are associated with the same disease or condition, such as polypeptides that are expressed by the same pathogen or type of pathogen, or associated with the same cancer or type of cancer, such as those disclosed herein. The method may then provide an ideal set of peptides from which to select peptides to treat the disease or condition in a specific subject of a given human population.

### Panels of Peptides

In some cases the disclosure provides a panel of peptides or a panel of polynucleic acids or vectors encoding a panel of peptides. The panel may be suitable for use in a method of inducing a T cell response against one or more target polypeptides in a subject of an intent-to-treat human population. The intent-to-treat human population may be a population as described herein and may be defined by the HLA genotype distribution in the subjects of the intent-to-treat population as described herein.

In some cases the panel is a panel designed and/or prepared according to the methods described herein. In other cases the panel comprises or encodes two or more peptides designed and/or prepared according to the method described herein.

In other cases the panel comprises or encodes two or more peptides, wherein each peptide comprises a fragment of the one or more target polypeptide, wherein the fragment comprises, in a high proportion of the intent-to-treat population, a sequence that meets any of the HLA-binding criteria described herein. In some cases a “high” percentage may be at least or more than 1%, 2%, 5%, 10%, 12%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49% or 50% of an intent-to-treat population as described herein.

The peptides of the panel may have any of the characteristics of a peptide described herein. For example, each peptide may be 9-50 amino acids in length; may comprise a fragment of the one or more target polypeptides that is 9-50 amino acids in length and meets the HLA-binding requirements; the target polypeptide fragment may be flanked at the N and/or C terminus of the peptide by additional amino acids that are not part of the consecutive sequence of the target polypeptide antigen; and/or the target polypeptide(s) may be any described herein, for example any of those listed in Tables 2 to 5.

In some cases the target polypeptide of each peptide of the panel may be the same; i.e each peptide comprises a different fragment of the target polypeptide, each of which meets the HLA-binding requirements in a high proportion of the intent-to-treat population. The panel then represents a selection of peptides that may be used to induce T cell responses against the same target polypeptide in different HLA-matched subjects. In some cases the fragments of the target polypeptide in the peptides of the panel do not overlap or do not comprise any common T cell epitopes or PEPIs.

In other cases the panel may comprise peptides that are designed to induce T cell responses against different target polypeptides, that is the selected fragments of the target polypeptides comprised in the peptides are from different target polypeptides. In some cases the panel comprises such fragments from at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 different target polypeptides.

The different target polypeptides may be any different polypeptides that it is useful to target or that can be selectively targeted with different PEPIs as described herein. In some cases different target polypeptide antigens are non-homologues or non-paralogues or have less than 95%, or 90%, or 85% or 80% or 75% or 70% or 60% or 50% sequence identity across the full length of each polypeptide.

In some cases the different target polypeptides targeted by the peptides of a panel are each expressed by or associated with the same disease, condition, pathogen or cancer, such as any described herein. Such a panel of pepides may be ideal for use in treatment of the disease or condition in a subject in need thereof, particularly if the peptides are HLA/PEPI matched to the specific subject as described herein.

In some cases one or more or each of the target polypeptides is present in a sample taken from a human subject. This indicates that the polypeptide(s) are expressed in the subject, for example a cancer- or tumor-associated antigen, TSA or CTA expressed by cancer cells of the subject.

In some cases 1, 2, 3, 4, 5, 6, 7, 8, 9, 10, 11, 12, 13, 14, 15, 16, 17, 18, 19, 20, 21, 22, 23, 24, or 25 or more or each of the target polypeptide antigens is a TSA and/or a CTA.

### Selection of Polypeptides and Patients

The peptides described herein may be used to induce T cell responses or provide vaccination or immunotherapy in a subject in need therefore. More than one peptide will typically be selected for treatment of a subject. Each peptide may be selected for treatment of a subject based on (i) the disease or condition to be treated in the subject; and/or (ii) the HLA genotype of the subject.

Each peptide selected for treatment of a subject may comprise a fragment as described herein of a target polypeptide antigen that is associated with the disease or condition to be treated in the subject, or expressed by target cells of the treatment, such as cancer cells. The disease or condition and the target polypeptide antigens may be any described herein. Typically each peptide selected for treatment of the subject will comprise a fragment as described herein of a different target polypeptide antigen. The target polypeptide antigens may be selected because they are known to be expressed by target cells in the subject. For example the target polypeptide antigens may have been detected in a sample obtained from the subject, such as a tumor biopsy. In other cases, the target polypeptide antigens may be selected based on their expression rate in the cells that are targeted by the treatment, for example the expression rate of a particular TAA in cancer or a particular type of cancer, such as any described herein. Typically the peptides selected for the treatment of the subject are those that comprise a fragment as described herein of the polypeptide antigens associated with the condition at the highest expression rates for the condition to be treated. Further the fragments typically have been predicted to induce a T cell response in the specific subject, as further described herein.

Polypeptide antigens, and particularly short peptides derived from polypeptide antigens, that are commonly used in vaccination and immunotherapy, induce immune responses in only a fraction of human subjects. The peptides of the present disclosure are specifically selected to induce immune responses in a high proportion of the general population, or a high proportion of a given intent-to-treat population. However, but they may not be effective in all individuals or all subjects of the intent-to-treat population due to HLA genotype heterogeneity.

In some cases the present disclosure provides a method of predicting that a specific human subject will have a T cell response (cytotoxic and/or helper) to administration of any of the peptides, panels of peptides or pharmaceutical compositions or kits described herein. As provided herein T cell epitope presentation by multiple HLAs of an individual is generally needed to trigger a T cell response. The best predictor of a cytotoxic (CD8+) T cell response to a given polypeptide is the presence of at least one T cell epitope that is presented by at least three HLA class I alleles of a subject (≥1 PEPI3+). Similarly the presence of at least one T cell epitope that is presented by at least three or four HLA class II alleles of a subject may be predictive of a helper (CD4+) T cell response. If such T cell epitopes correspond to a fragment of a target polypeptide antigen, such as any target polypeptide antigen described herein, then the subject is predicted to mount a T cell response that targets cells in the subject that express the target polypeptide, if present. Accordingly in some cases the method may be for predicting a T cell response in a subject to a target polypeptide antigen, such as any described herein.

The inventors have further discovered that the presence in a vaccine or immunotherapy composition of at least two T cell epitopes that (i) correspond to fragments of one or more target polypeptide antigens, and (ii) can bind to at least three HLA class I alleles of an individual is predictive for a clinical response. For example, if an individual has a total of ≥2 PEPI3+ within the active ingredient peptide(s) of a vaccine or immunotherapy composition, and these PEPI3+s are derived from polypeptide antigens that are in fact expressed by target cells in the individual (for example, target tumor cells of the individual express the target tumor-associated antigens), then the individual is a likely clinical responder (i.e. a clinically relevant immune responder).

A “clinical response” or “clinical benefit” as used herein may be the prevention or a delay in the onset of a disease or condition, the amelioration of one or more symptoms, the induction or prolonging of remission, or the delay of a relapse or recurrence or deterioration, or any other improvement or stabilisation in the disease status of a subject. Where appropriate, a “clinical response” may correlate to “disease control” or an “objective response” as defined by the Response Evaluation Criteria In Solid Tumors (RECIST) guidelines.

Accordingly some aspects of the disclosure relate to a method of predicting that a specific human subject will have a clinical response to a method of treatment as described herein or to administration of a pharmaceutical composition or the peptides, nucleic acids or vectors of a pharmaceutical kit described herein.

In some cases the method comprises determining that the active ingredient peptide(s) for treatment of the subject comprise two or more different amino acid sequences each of which is a) a fragment of a target polypeptide antigen expressed by target cells of the subject (for example, polypeptide antigens that have been detected in a biopsy); and b) a T cell epitope capable of binding to at least three HLA class I of the subject.

In some cases the likelihood that a subject will have a clinical response to a peptide vaccine or immunotherapy composition, such as those described herein, can be determined without knowing whether the target antigens are expressed in target cells, such as cancer cells of the subject and/or without determining the HLA class I genotype of the subject. Known antigen expression frequencies in the disease (e.g. MAGE-A3 in a tumor type like gastric cancer) and/or known frequencies for HLA class I and class II genotype of subjects in the target population (e.g. ethnic population, general population, diseased population) may be used instead.

The likelihood that a subject will respond to treatment is increased by (i) the presence of more multiple HLA-binding PEPIs in the active ingredient polypeptides; (ii) the presence of PEPIs in more target polypeptide antigens; and (iii) expression of the target polypeptide antigens in the subject or in diseased cells of the subject. The probability that target cells in the subject (over-)express a specific or any combination of target polypeptide antigens may be determined using population expression frequency data (expression rates), e.g. probability of expression of an antigen in gastric cancer. The population expression frequency data may relate to a subject- and/or disease-matched population or the intent-to-treat population. For example, the frequency or probability of expression of a particular cancer-associated antigen in a particular cancer or subject having a particular cancer, for example breast cancer, can be determined by detecting the antigen in tumor, e.g. breast cancer tumor samples. Such expression frequencies may be determined from published figures and scientific publications. In some cases a method of the disclosure may comprise a step of determining the expression frequency of a relevant target polypeptide antigen in a relevant population.

Disclosed is a range of pharmacodynamic biomarkers to predict the activity/effect of vaccines in individual human subjects as well as in populations of human subjects. These biomarkers expedite more effective vaccine development and also decrease the development cost and may be used to assess and compare different compositions. Exemplary biomarkers are as follows.
AG95—potency of a vaccine: The number of antigens in a cancer vaccine that a specific tumor type expresses with 95% probability. AG95 is an indicator of the vaccine's potency, and is independent of the immunogenicity of the vaccine antigens. AG95 is calculated from the tumor antigen expression rate data. Such data may be obtained from experiments published in peer reviewed scientific journals. Technically, AG95 is determined from the binomial distribution of antigens in the vaccine, and takes into account all possible variations and expression rates.
PEPI3+ count—immunogenicity of a vaccine in a subject: Vaccine-derived PEPI3+ are personal epitopes that bind to at least 3 HLAs of a subject and induce T cell responses. PEPI3+ can be determined using the PEPI3+ Test in subjects who's complete 4-digit HLA genotype is known.
AP count—antigenicity of a vaccine in a subject: Number of vaccine antigens with PEPI3+. Vaccines contain sequences from target polypeptide antigens expressed by diseased cells. AP count is the number of antigens in the vaccine that contain PEPI3+, and the AP count represents the number of antigens in the vaccine that can induce T cell responses in a subject. AP count characterizes the vaccine-antigen specific T cell responses of the subject since it depends only on the HLA genotype of the subject and is independent of the subject's disease, age, and medication. The correct value is between 0 (no PEPI presented by the antigen) and maximum number of antigens (all antigens present PEPIs).
AP50—antigenicity of a vaccine in a population: The mean number of vaccine antigens with a PEPI in a population. The AP50 is suitable for the characterization of vaccine-antigen specific T cell responses in a given population since it depends on the HLA genotype of subjects in a population.
AGP count—effectiveness of a vaccine in a subject: Number of vaccine antigens expressed in the tumor with PEPI. The AGP count indicates the number of tumor antigens that vaccine recognizes and induces a T cell response against (hit the target). The AGP count depends on the vaccine-antigen expression rate in the subject's tumor and the HLA genotype of the subject. The correct value is between 0 (no PEPI presented by expressed antigen) and maximum number of antigens (all antigens are expressed and present a PEPI).
AGP50—effectiveness of a cancer vaccine in a population: The mean number of vaccine antigens expressed in the indicated tumor with PEPI (i.e., AGP) in a population. The AGP50 indicates the mean number of tumor antigens that the T cell responses induced by the vaccine can recognize. AGP50 is dependent on the expression rate of the antigens in the indicated tumor type and the immunogenicity of the antigens in the target population. AGP50 can estimate a vaccine's effectiveness in different populations and can be used to compare different vaccines in the same population. The computation of AGP50 is similar to that used for AG50, except the expression is weighted by the occurrence of the PEPI3+ in the subject on the expressed vaccine antigens. In a theoretical population, where each subject has a PEPI from each vaccine antigen, the AGP50 will be equal to AG50. In another theoretical population, where no subject has a PEPI from any vaccine antigen, the AGP50 will be 0. In general, the following statement is valid: 0≤AGP50≤AG50.
mAGP—a candidate biomarker for the selection of likely responders: Likelihood that a cancer vaccine induces T cell responses against multiple antigens expressed in the indicated tumor. mAGP is calculated from the expression rates of vaccine-antigens in the tumor and the presence of vaccine derived PEPIs in the subject. Technically, based on the AGP distribution, the mAGP is the sum of probabilities of the multiple AGP (≥2 AGPs).

The results of a prediction as set out above may be used to inform a physician's decisions concerning treatment of the subject. Accordingly, in some cases the method of the disclosure predicts that a subject will have or is likely to have a T cell response and/or a clinical response to a treatment as described herein, and the method further comprises selecting the treatment for the human subject. In some cases a subject is selected for treatment if their likelihood of a response targeted at a predefined number of target polypeptide antigens, optionally wherein the target polypeptide antigens are (predicted to be) expressed, is above a predetermined threshold. In some cases the number of target polypeptide antigens or epitopes is two. In some cases the number of target polypeptide antigens or epitopes is three, or four, or five, or six, or seven, or eight, or nine, or ten. The method may further comprise administering the treatment to the human subject. Alternatively, the method may predict that the subject will not have an immune response and/or a clinical response and further comprise selecting a different treatment for the subject.

### Pharmaceutical Compositions, Methods of Treatment and Modes of Administration

In some aspects the disclosure relates to a pharmaceutical composition or kit comprising one or more of the peptides, polynucleic acids or vectors described herein. Such pharmaceutical compositions or kits may be for use in a method of inducing an immune response, treating, vaccinating or providing immunotherapy to a subject. The pharmaceutical composition or kit may be a vaccine or immunotherapy composition or kit. Such treatment may comprise administering the pharmaceutical composition or the peptides, polynucleic acids or vectors of the kit to the subject.

The pharmaceutical compositions or kits described herein may comprise, in addition to one or more peptides, nucleic acids or vectors, a pharmaceutically acceptable excipient, carrier, diluent, buffer, stabiliser, preservative, adjuvant or other materials well known to those skilled in the art. Such materials are preferably non-toxic and preferably do not interfere with the pharmaceutical activity of the active ingredient(s). The pharmaceutical carrier or diluent may be, for example, water containing solutions. The precise nature of the carrier or other material may depend on the route of administration, e.g. oral, intravenous, cutaneous or subcutaneous, nasal, intramuscular, intradermal, and intraperitoneal routes.

The pharmaceutical compositions of the disclosure may comprise one or more “pharmaceutically acceptable carriers”. These are typically large, slowly metabolized macromolecules such as proteins, saccharides, polylactic acids, polyglycolic acids, polymeric amino acids, amino acid copolymers, sucrose (Paoletti et al., 2001, Vaccine, 19:2118), trehalose (WO 00/56365), lactose and lipid aggregates (such as oil droplets or liposomes). Such carriers are well known to those of ordinary skill in the art. The pharmaceutical compositions may also contain diluents, such as water, saline, glycerol, etc. Additionally, auxiliary substances, such as wetting or emulsifying agents, pH buffering substances, and the like, may be present. Sterile pyrogen-free, phosphate buffered physiologic saline is a typical carrier (Gennaro, 2000, Remington: The Science and Practice of Pharmacy, 20th edition, ISBN:0683306472).

The pharmaceutical compositions of the disclosure may be lyophilized or in aqueous form, i.e. solutions or suspensions. Liquid formulations of this type allow the compositions to be administered direct from their packaged form, without the need for reconstitution in an aqueous medium, and are thus ideal for injection. The pharmaceutical compositions may be presented in vials, or they may be presented in ready filled syringes. The syringes may be supplied with or without needles. A syringe will include a single dose, whereas a vial may include a single dose or multiple doses.

Liquid formulations of the disclosure are also suitable for reconstituting other medicaments from a lyophilized form. Where a pharmaceutical composition is to be used for such extemporaneous reconstitution, the disclosure provides a kit, which may comprise two vials, or may comprise one ready-filled syringe and one vial, with the contents of the syringe being used to reconstitute the contents of the vial prior to injection.

The pharmaceutical compositions of the disclosure may include an antimicrobial, particularly when packaged in a multiple dose format. Antimicrobials may be used, such as 2-phenoxyethanol or parabens (methyl, ethyl, propyl parabens). Any preservative is preferably present at low levels. Preservative may be added exogenously and/or may be a component of the bulk antigens which are mixed to form the composition (e.g. present as a preservative in pertussis antigens).

The pharmaceutical compositions of the disclosure may comprise detergent e.g. Tween (polysorbate), DMSO (dimethyl sulfoxide), DMF (dimethylformamide). Detergents are generally present at low levels, e.g. <0.01%, but may also be used at higher levels, e.g. 0.01-50%.

The pharmaceutical compositions of the disclosure may include sodium salts (e.g. sodium chloride) and free phosphate ions in solution (e.g. by the use of a phosphate buffer).

In certain embodiments, the pharmaceutical composition may be encapsulated in a suitable vehicle either to deliver the peptides into antigen presenting cells or to increase the stability. As will be appreciated by a skilled artisan, a variety of vehicles are suitable for delivering a pharmaceutical composition of the disclosure. Non-limiting examples of suitable structured fluid delivery systems may include nanoparticles, liposomes, microemulsions, micelles, dendrimers and other phospholipid-containing systems. Methods of incorporating pharmaceutical compositions into delivery vehicles are known in the art.

In order to increase the immunogenicity of the composition, the pharmacological compositions may comprise one or more adjuvants and/or cytokines.

Suitable adjuvants include an aluminum salt such as aluminum hydroxide or aluminum phosphate, but may also be a salt of calcium, iron or zinc, or may be an insoluble suspension of acylated tyrosine, or acylated sugars, or may be cationically or anionically derivatised saccharides, polyphosphazenes, biodegradable microspheres, monophosphoryl lipid A (MPL), lipid A derivatives (e.g. of reduced toxicity), 3-O-deacylated MPL [3D-MPL], quil A, Saponin, QS21, Freund's Incomplete Adjuvant (Difco Laboratories, Detroit, Mich.), Merck Adjuvant 65 (Merck and Company, Inc., Rahway, N.J.), AS-2 (Smith-Kline Beecham, Philadelphia, Pa.), CpG oligonucleotides, bioadhesives and mucoadhesives, microparticles, liposomes, polyoxyethylene ether formulations, polyoxyethylene ester formulations, muramyl peptides or imidazoquinolone compounds (e.g. imiquamod and its homologues). Human immunomodulators suitable for use as adjuvants in the disclosure include cytokines such as interleukins (e.g. IL-1, IL-2, IL-4, IL-5, IL-6, IL-7, IL-12, etc), macrophage colony stimulating factor (M-CSF), tumour necrosis factor (TNF), granulocyte, macrophage colony stimulating factor (GM-CSF) may also be used as adjuvants.

In some embodiments, the compositions comprise an adjuvant selected from the group consisting of Montanide ISA-51 (Seppic, Inc., Fairfield, N.J., United States of America), QS-21 (Aquila Biopharmaceuticals, Inc., Lexington, Mass., United States of America), GM-CSF, cyclophosamide, bacillus Calmette-Guerin (BCG), corynbacterium parvum, levamisole, azimezone, isoprinisone, dinitrochlorobenezene (DNCB), keyhole limpet hemocyanins (KLH), Freunds adjuvant (complete and incomplete), mineral gels, aluminum hydroxide (Alum), lysolecithin, pluronic polyols, polyanions, oil emulsions, dinitrophenol, diphtheria toxin (DT).

By way of example, the cytokine may be selected from the group consisting of a transforming growth factor (TGF) such as but not limited to TGF-α and TGF-β; insulin-like growth factor-I and/or insulin-like growth factor-II; erythropoietin (EPO); an osteoinductive factor; an interferon such as but not limited to interferon-.α, -β, and -γ; a colony stimulating factor (CSF) such as but not limited to macrophage-CSF (M-CSF); granulocyte-macrophage-CSF (GM-CSF); and granulocyte-CSF (G-CSF). In some embodiments, the cytokine is selected from the group consisting of nerve growth factors such as NGF-β; platelet-growth factor; a transforming growth factor (TGF) such as but not limited to TGF-α. and TGF-β; insulin-like growth factor-I and insulin-like growth factor-II; erythropoietin (EPO); an osteoinductive factor; an interferon (IFN) such as but not limited to IFN-α, IFN-β, and IFN-γ; a colony stimulating factor (CSF) such as macrophage-CSF (M-CSF); granulocyte-macrophage-CSF (GM-CSF); and granulocyte-CSF (G-CSF); an interleukin (I1) such as but not limited to IL-1, IL-1.alpha., IL-2, IL-3, IL-4, IL-5, IL-6, IL-7, IL-8, IL-9, IL-10, IL-11, IL-12; IL-13, IL-14, IL-15, IL-16, IL-17, IL-18; LIF; kit-ligand or FLT-3; angiostatin; thrombospondin; endostatin; a tumor necrosis factor (TNF); and LT.

It is expected that an adjuvant or cytokine can be added in an amount of about 0.01 mg to about 10 mg per dose, preferably in an amount of about 0.2 mg to about 5 mg per dose. Alternatively, the adjuvant or cytokine may be at a concentration of about 0.01 to 50%, preferably at a concentration of about 2% to 30%.

In certain aspects, the pharmaceutical compositions of the disclosure are prepared by physically mixing the adjuvant and/or cytokine with the PEPIs under appropriate sterile conditions in accordance with known techniques to produce the final product.

Examples of suitable compositions of polypeptide fragments and methods of administration are provided in Esseku and Adeyeye (2011) and Van den Mooter G. (2006). Vaccine and immunotherapy composition preparation is generally described in Vaccine Design (“The subunit and adjuvant approach” (eds Powell M. F. & Newman M. J. (1995) Plenum Press New York). Encapsulation within liposomes, which is also envisaged, is described by Fullerton, U.S. Pat. No. 4,235,877.

In some embodiments, the compositions disclosed herein are prepared as a nucleic acid vaccine. In some embodiments, the nucleic acid vaccine is a DNA vaccine. In some embodiments, DNA vaccines, or gene vaccines, comprise a plasmid with a promoter and appropriate transcription and translation control elements and a nucleic acid sequence encoding one or more polypeptides of the disclosure. In some embodiments, the plasmids also include sequences to enhance, for example, expression levels, intracellular targeting, or proteasomal processing. In some embodiments, DNA vaccines comprise a viral vector containing a nucleic acid sequence encoding one or more polypeptides of the disclosure. In additional aspects, the compositions disclosed herein comprise one or more nucleic acids encoding peptides determined to have immunoreactivity with a biological sample. For example, in some embodiments, the compositions comprise one or more nucleotide sequences encoding 1, 2, 3, 4, 5, 6, 7, 8, 9, 10, 11, 12, 13, 14, 15, 16, 17, 18, 19, 20, or more peptides comprising a fragment that is a T cell epitope capable of binding to at least three HLA class I molecules and/or at least three or four HLA class II molecules of a patient. In some embodiments, the peptides are derived from an antigen that is expressed in cancer. In some embodiments the DNA or gene vaccine also encodes immunomodulatory molecules to manipulate the resulting immune responses, such as enhancing the potency of the vaccine, stimulating the immune system or reducing immunosuppression. Strategies for enhancing the immunogenicity of of DNA or gene vaccines include encoding of xenogeneic versions of antigens, fusion of antigens to molecules that activate T cells or trigger associative recognition, priming with DNA vectors followed by boosting with viral vector, and utilization of immunomodulatory molecules. In some embodiments, the DNA vaccine is introduced by a needle, a gene gun, an aerosol injector, with patches, via microneedles, by abrasion, among other forms. In some forms the DNA vaccine is incorporated into liposomes or other forms of nanobodies. In some embodiments, the DNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nanoparticle. In some embodiments, the DNA vaccines are administered by inhalation or ingestion. In some embodiments, the DNA vaccine is introduced into the blood, the thymus, the pancreas, the skin, the muscle, a tumor, or other sites.

In some embodiments, the compositions disclosed herein are prepared as an RNA vaccine. In some embodiments, the RNA is non-replicating mRNA or virally derived, self-amplifying RNA. In some embodiments, the non-replicating mRNA encodes the peptides disclosed herein and contains 5′ and 3′ untranslated regions (UTRs). In some embodiments, the virally derived, self-amplifying RNA encodes not only the peptides disclosed herein but also the viral replication machinery that enables intracellular RNA amplification and abundant protein expression. In some embodiments, the RNA is directly introduced into the individual. In some embodiments, the RNA is chemically synthesized or transcribed in vitro. In some embodiments, the mRNA is produced from a linear DNA template using a T7, a T3, or an Sp6 phage RNA polymerase, and the resulting product contains an open reading frame that encodes the peptides disclosed herein, flanking UTRs, a 5′ cap, and a poly(A) tail. In some embodiments, various versions of 5′ caps are added during or after the transcription reaction using a vaccinia virus capping enzyme or by incorporating synthetic cap or anti-reverse cap analogues. In some embodiments, an optimal length of the poly(A) tail is added to mRNA either directly from the encoding DNA template or by using poly(A) polymerase. The RNA may encode one or more peptides comprising a fragment that is a T cell epitope capable of binding to at least three HLA class I and/or at least three or four HLA class II molecules of a patient. In some embodiments, the fragments are derived from an antigen that is expressed in cancer. In some embodiments, the RNA includes signals to enhance stability and translation. In some embodiments, the RNA also includes unnatural nucleotides to increase the half-life or modified nucleosides to change the immunostimulatory profile. In some embodiments, the RNAs is introduced by a needle, a gene gun, an aerosol injector, with patches, via microneedles, by abrasion, among other forms. In some forms the RNA vaccine is incorporated into liposomes or other forms of nanobodies that facilitate cellular uptake of RNA and protect it from degradation. In some embodiments, the RNA vaccine includes a delivery system selected from the group consisting of a transfection agent; protamine; a protamine liposome; a polysaccharide particle; a cationic nanoemulsion; a cationic polymer; a cationic polymer liposome; a cationic nanoparticle; a cationic lipid and cholesterol nanoparticle; a cationic lipid, cholesterol, and PEG nanoparticle; a dendrimer nanoparticle; and/or naked mRNA; naked mRNA with in vivo electroporation; protamine-complexed mRNA; mRNA associated with a positively charged oil-in-water cationic nanoemulsion; mRNA associated with a chemically modified dendrimer and complexed with polyethylene glycol (PEG)-lipid; protamine-complexed mRNA in a PEG-lipid nanoparticle; mRNA associated with a cationic polymer such as polyethylenimine (PH); mRNA associated with a cationic polymer such as PEI and a lipid component; mRNA associated with a polysaccharide (for example, chitosan) particle or gel; mRNA in a cationic lipid nanoparticle (for example, 1,2-dioleoyloxy-3-trimethylammoniumpropane (DOT AP) or dioleoylphosphatidylethanolamine (DOPE) lipids); mRNA complexed with cationic lipids and cholesterol; or mRNA complexed with cationic lipids, cholesterol and PEG-lipid. In some embodiments, the RNA vaccine is administered by inhalation or ingestion. In some embodiments, the RNA is introduced into the blood, the thymus, the pancreas, the skin, the muscle, a tumor, or other sites, and/or by an intradermal, intramuscular, subcutaneous, intranasal, intranodal, intravenous, intrasplenic, intratumoral or other delivery route.

Polynucleotide or oligonucleotide components may be naked nucleotide sequences or be in combination with cationic lipids, polymers or targeting systems. They may be delivered by any available technique. For example, the polynucleotide or oligonucleotide may be introduced by needle injection, preferably intradermally, subcutaneously or intramuscularly. Alternatively, the polynucleotide or oligonucleotide may be delivered directly across the skin using a delivery device such as particle-mediated gene delivery. The polynucleotide or oligonucleotide may be administered topically to the skin, or to mucosal surfaces for example by intranasal, oral, or intrarectal administration.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectam and transfectam. The dosage of the polynucleotide or oligonucleotide to be administered can be altered.

Administration is typically in a “prophylactically effective amount” or a “therapeutically effective amount” (as the case may be, although prophylaxis may be considered therapy), this being sufficient to result in a clinical response or to show clinical benefit to the individual, e.g. an effective amount to prevent or delay onset of the disease or condition, to ameliorate one or more symptoms, to induce or prolong remission, or to delay relapse or recurrence.

The dose may be determined according to various parameters, especially according to the substance used; the age, weight and condition of the individual to be treated; the route of administration; and the required regimen. The amount of antigen in each dose is selected as an amount which induces an immune response. A physician will be able to determine the required route of administration and dosage for any particular individual. The dose may be provided as a single dose or may be provided as multiple doses, for example taken at regular intervals, for example 2, 3 or 4 doses administered hourly. Typically peptides, polynucleotides or oligonucleotides are typically administered in the range of 1 pg to 1 mg, more typically 1 pg to 10 μg for particle mediated delivery and 1 μg to 1 mg, more typically 1-100 μg, more typically 5-50 μg for other routes. Generally, it is expected that each dose will comprise 0.01-3 mg of antigen. An optimal amount for a particular vaccine can be ascertained by studies involving observation of immune responses in subjects.

Examples of the techniques and protocols mentioned above can be found in Remington's Pharmaceutical Sciences, 20th Edition, 2000, pub. Lippincott, Williams & Wilkins.

In some cases the method of treatment may comprise administration to a subject of more than one peptide, polynucleic acid or vector. These may be administered together/simultaneously and/or at different times or sequentially. The use of combinations of different peptides, optionally targeting different antigens, may be important to overcome the challenges of genetic heterogeneity of tumors and HLA heterogeneity of individuals. The use of peptides of the disclosure in combination expands the group of individuals who can experience clinical benefit from vaccination. Multiple pharmaceutical compositions of PEPIs, manufactured for use in one regimen, may define a drug product. In some cases different peptides, polynucleic acids or vectors of a single treatment may be administered to the subject within a period of, for example, 1 year, or 6 months, or 3 months, or 60 or 50 or 40 or 30 days.

Routes of administration include but are not limited to intranasal, oral, subcutaneous, intradermal, and intramuscular. The subcutaneous administration is particularly preferred. Subcutaneous administration may for example be by injection into the abdomen, lateral and anterior aspects of upper arm or thigh, scapular area of back, or upper ventrodorsal gluteal area.

The compositions of the disclosure may also be administered in one, or more doses, as well as, by other routes of administration. For example, such other routes include, intracutaneously, intravenously, intravascularly, intraarterially, intraperitnoeally, intrathecally, intratracheally, intracardially, intralobally, intramedullarly, intrapulmonarily, and intravaginally. Depending on the desired duration of the treatment, the compositions according to the disclosure may be administered once or several times, also intermittently, for instance on a monthly basis for several months or years and in different dosages.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

One or more compositions of the disclosure may be administered, or the methods and uses for treatment according to the disclosure may be performed, alone or in combination with other pharmacological compositions or treatments, for example chemotherapy and/or immunotherapy and/or vaccine. The other therapeutic compositions or treatments may for example be one or more of those discussed herein, and may be administered either simultaneously or sequentially with (before or after) the composition or treatment of the disclosure.

In some cases the treatment may be administered in combination with checkpoint blockade therapy/checkpoint inhibitors, co-stimulatory antibodies, cytotoxic or non-cytotoxic chemotherapy and/or radiotherapy, targeted therapy or monoclonal antibody therapy. It has been demonstrated that chemotherapy sensitizes tumors to be killed by tumor specific cytotoxic T cells induced by vaccination (Ramakrishnan et al. J Clin Invest. 2010; 120(4):1111-1124). Examples of chemotherapy agents include alkylating agents including nitrogen mustards such as mechlorethamine (HN2), cyclophosphamide, ifosfamide, melphalan (L-sarcolysin) and chlorambucil; anthracyclines; epothilones; nitrosoureas such as carmustine (BCNU), lomustine (CCNU), semustine (methyl-CCNU) and streptozocin (streptozotocin); triazenes such as decarbazine (DTIC; dimethyltriazenoimidazole-carboxamide; ethylenimines/methylmelamines such as hexamethylmelamine, thiotepa; alkyl sulfonates such as busulfan; Antimetabolites including folic acid analogues such as methotrexate (amethopterin); alkylating agents, antimetabolites, pyrimidine analogs such as fluorouracil (5-fluorouracil; 5-FU), floxuridine (fluorodeoxyuridine; FUdR) and cytarabine (cytosine arabinoside); purine analogues and related inhibitors such as mercaptopurine (6-mercaptopurine; 6-MP), thioguanine (6-thioguanine; TG) and pentostatin (2′-deoxycoformycin); epipodophylotoxins; enzymes such as L-asparaginase; biological response modifiers such as IFNα, IL-2, G-CSF and GM-CSF; platinum coordination complexes such as cisplatin (cis-DDP), oxaliplatin and carboplatin; anthracenediones such as mitoxantrone and anthracycline; substituted urea such as hydroxyurea; methylhydrazine derivatives including procarbazine (N-methylhydrazine, MIH) and procarbazine; adrenocortical suppressants such as mitotane (o,p′-DDD) and aminoglutethimide; taxol and analogues/derivatives; hormones/hormonal therapy and agonists/antagonists including adrenocorticosteroid antagonists such as prednisone and equivalents, dexamethasone and aminoglutethimide, progestin such as hydroxyprogesterone caproate, medroxyprogesterone acetate and megestrol acetate, estrogen such as diethylstilbestrol and ethinyl estradiol equivalents, antiestrogen such as tamoxifen, androgens including testosterone propionate and fluoxymesterone/equivalents, antiandrogens such as flutamide, gonadotropin-releasing hormone analogs and leuprolide and non-steroidal antiandrogens such as flutamide; natural products including vinca alkaloids such as vinblastine (VLB) and vincristine, epipodophyllotoxins such as etoposide and teniposide, antibiotics such as dactinomycin (actinomycin D), daunorubicin (daunomycin; rubidomycin), doxorubicin, bleomycin, plicamycin (mithramycin) and mitomycin (mitomycin C), enzymes such as L-asparaginase, and biological response modifiers such as interferon alphenomes.

In some cases the method of treatment is a method of vaccination or a method of providing immunotherapy. As used herein, “immunotherapy” is the treatment of a disease or condition by inducing or enhancing an immune response in an individual. In certain embodiments, immunotherapy refers to a therapy that comprises the administration of one or more drugs to an individual to elicit T cell responses. In a specific embodiment, immunotherapy refers to a therapy that comprises the administration or expression of polypeptides that contain one or more PEPIs to an individual to elicit a T cell response to recognize and kill cells that display the one or more PEPIs on their cell surface in conjunction with a class I HLA. In another specific embodiment, immunotherapy comprises the administration of one or more PEPIs to an individual to elicit a cytotoxic T cell response against cells that display tumor associated antigens (TAAs), tumor specific antigens (TSAs) or cancer testis antigens (CTAs) comprising the one or more PEPIs on their cell surface. In another embodiment, immunotherapy refers to a therapy that comprises the administration or expression of polypeptides that contain one or more PEPIs presented by class II HLAs to an individual to elicit a T helper response to provide co-stimulation to cytotoxic T cells that recognize and kill diseased cells that display the one or more PEPIs on their cell surface in conjunction with a class I HLAs. In still another specific embodiment, immunotherapy refers to a therapy that comprises administration of one or more drugs to an individual that re-activate existing T cells to kill target cells. The theory is that the cytotoxic T cell response will eliminate the cells displaying the one or more PEPIs, thereby improving the clinical condition of the individual. In some instances, immunotherapy may be used to treat tumors. In other instances, immunotherapy may be used to treat intracellular pathogen-based diseases or disorders.

In some cases the disclosure relates to the treatment of cancer or any specific type of cancer described herein. In some other cases the disclosure relates to the treatment of a viral, bacterial, fungal or parasitic infection, or any other disease or condition that may be treated by immunotherapy.

### FURTHER EMBODIMENTS OF THE DISCLOSURE

1. A pharmaceutical composition, comprising two or more different peptides, wherein each peptide is up to 50 amino acids in length and comprises the amino acid sequence of any of SEQ ID NOs: 1 to 2786 and/or 5432 to 5931.
2. The pharmaceutical composition of item 1, comprising at least 3, at least 4, at least 5, at least 6, at least 7, at least 8, at least 9, at least 10, at least 11, or at least 12 different peptides, wherein each peptide is up to 50 amino acids in length and comprises the amino acid sequence of any of SEQ ID Nos: 1 to 2786 and/or 5432 to 5931.
3. A pharmaceutical composition comprising one or more polynucleic acids or vectors that encode two or more peptides wherein each peptide is up to 50 amino acids in length and comprises the amino acid sequence of any of SEQ ID NOs: 1 to 2786 and/or 5432 to 5931.
4. The pharmaceutical composition of item 2 comprising at least 2, at least 3, at least 4, at least 5, at least 6, at least 7, at least 8, at least 9, at least 10, at least 11, or at least 12 polynucleic acids or vectors.
5. The pharmaceutical composition of item 1 or item 3, wherein each peptide or encoded peptide comprises at least one amino acid sequence selected from one of the following groups:
(a) the sequences listed in Table 25A and/or 25B and indicated in Table 25A or 25B to be a fragment of a breast cancer-associated antigen listed in Table 24;
(b) the sequences listed in Table 25A and/or Table 25B and indicated in Table 25A or 25B to be a fragment of a lung cancer-associated antigen listed in Table 24;
(c) the sequences listed in Table 25A and/or Table 25B and indicated in Table 25A or 25B to be a fragment of a prostate cancer-associated antigen listed in Table 24;
(d) the sequences listed in Table 25A and/or Table 25B and indicated in Table 25A or 25B to be a fragment of a colorectal cancer-associated antigen listed in Table 24;
(e) the sequences listed in Table 25A and/or Table 25B and indicated in Table 25A or 25B to be a fragment of a bladder cancer-associated antigen listed in Table 24;
(f) the sequences listed in Table 25A and/or Table 25B and indicated in Table 25A or 25B to be a fragment of a ovarian cancer-associated antigen listed in Table 24;
(g) the sequences listed in Table 25A and/or Table 25B and indicated in Table 25A or 25B to be a fragment of a pancreatic cancer-associated antigen listed in Table 24;
(h) the sequences listed in Table 25A and/or Table 25B and indicated in Table 25A or 25B to be a fragment of a brain cancer-associated antigen listed in Table 24;
(i) the sequences listed in Table 25A and/or Table 25B and indicated in Table 25A or 25B to be a fragment of a leukemia-associated antigen listed in Table 24;
(j) the sequences listed in Table 25A and/or Table 25B and indicated in Table 25A or 25B to be a fragment of a lymphoma-associated antigen listed in Table 24;
(k) the sequences listed in Table 25A and/or Table 25B and indicated in Table 25A or 25B to be a fragment of a hepatocellular cancer-associated antigen listed in Table 24;
(l) the sequences listed in Table 25A and/or Table 25B and indicated in Table 25A or 25B to be a fragment of a melanoma-associated antigen listed in Table 24;
(m) the sequences listed in Table 25A and/or Table 25B and indicated in Table 25A or 25B to be a fragment of a thyroid cancer-associated antigen listed in Table 24;
(n) the sequences listed in Table 25A and/or Table 25B and indicated in Table 25A or 25B to be a fragment of a pediatric cancer-associated antigen listed in Table 24;
(o) the sequences listed in Table 25A and/or Table 25B and indicated in Table 25A or 25B to be a fragment of a gastric cancer-associated antigen listed in Table 24;
(p) the sequences listed in Table 25A and/or Table 25B and indicated in Table 25A or 25B to be a fragment of a kidney cancer-associated antigen listed in Table 24;
(q) the sequences listed in Table 25A and/or Table 25B and indicated in Table 25A or 25B to be a fragment of a head and neck cancer-associated antigen listed in Table 24; and
(r) the sequences listed in Table 25A and/or Table 25B and indicated in Table 25A or 25B to be a fragment of a cervical cancer-associated antigen listed in Table 24.
6. The pharmaceutical composition according to any of items 1-5 further comprising a pharmaceutically acceptable adjuvant, diluent, carrier, preservative, or a combination thereof
7. A kit comprising:
   a. a first pharmaceutical composition comprising one or more peptides, wherein each peptide comprises a different one of the amino acid sequence of any one of SEQ ID NOs: 1 to 2786 and/or 5432 to 5931; and
   b. a second different pharmaceutical composition comprising one or more peptides, wherein each peptide comprises a different one of the amino acid sequence of any one of SEQ ID NOs: 1 to 2786 and/or 5432 to 5931.
8. The kit of item 7, further comprising a package insert.
9. A method of inducing a cytotoxic T cell response and/or a helper T cell response in a subject of a target population, the method comprising administering a pharmaceutical composition according to any one of item 1 to item 7.
10. The method of item 9, further comprising prior to the administering step, determining if the subject is likely to have an have a clinical response to administration of the pharmaceutical composition by
   a. determining that each peptide, or encoded peptide of the pharmaceutical composition comprises at least one amino acid sequence that is a T cell epitope capable of binding to at least three HLA class I molecules of the subject; and
   b. predicting that the subject will have a cytotoxic T cell response to each peptide, each encoded peptide, polynucleic acid or vector of the pharmaceutical composition.
11. A method of vaccination, providing immunotherapy or inducing a cytotoxic T cell response in a subject, the method comprising administering to the subject a pharmaceutical composition according to any one of item 1 to item 6.
12. The method of item 11 wherein the peptides, polynucleic acids or vectors of the pharmaceutical composition have been predicted to induce a cytotoxic T cell response and/or a helper T cell response in the subject using a method comprising:
   a. determining that each peptide, or encoded peptide of the pharmaceutical composition comprises at least one amino acid sequence that is a T cell epitope capable of binding to at least three HLA class I molecules of the subject; and
   b. predicting that the subject will have a cytotoxic T cell response to each peptide, encoded peptide, polynucleic acid or vector of the pharmaceutical composition.
13. The method of item 12, wherein the peptides or encoded peptides of the pharmaceutical composition are fragments of two or more different cancer associated antigens selected from those listed in Table 22.
14. The method according to any one of item 9 to item 14 that is a method of treating cancer, optionally bladder cancer, brain cancer, breast cancer, colorectal cancer, gastric cancer, hepatocellular cancer, leukemia, lung cancer, lymphoma, melanoma, ovarian cancer, pancreatic cancer, pediatric cancer, thyroid cancer or prostate cancer.
15. A method of designing or preparing a peptide, or a polynucleic acid or vector that encodes
a peptide, or a panel of peptides, or one or more polynucleic acid or vectors that encode a panel of peptides, for use in a method of inducing a T cell response against a target polypeptide, the method comprising
   (i) selecting or defining a model human population comprising a plurality of subjects each defined by HLA class I genotype and/or by HLA class II genotype;
   (ii) identifying for each subject of the model population:
      (a) amino acid sequences of the target polypeptide that are a T cell epitope capable of binding to at least three HLA class I molecules of the subject;
      (b) amino acid sequences of the target polypeptide that are a T cell epitope capable of binding to at least four HLA class II molecules of the subject;
      (c) amino acid sequences of the target polypeptide that comprise a T cell epitope capable of binding to at least three HLA class I molecules of the subject and a T cell epitope capable of binding to at least four HLA class II molecules of the subject; or
      (d) amino acid sequences of the target polypeptide that both
         a. are a T cell epitope capable of binding to at least four HLA class II molecules; and
         b. comprise an amino acid sequence that is a T cell epitope capable of binding to at least three HLA class I molecules of the subject;
   (iii) selecting a polypeptide fragment window length of between 9 and 50 amino acids;
   (iv) identifying a fragment of the target polypeptide that
      (c) has the length selected in step (iii); and
      (d) comprises an amino acid sequence identified in any one of step (ii) (a) to (d) in the highest proportion of subjects in the model population;
   (v) optionally testing the fragment identified in step (iv) against additional pre-defined criteria, rejecting the fragment if the further pre-defined criteria are not met, and repeating step (iv) to identify an alternative fragment of the target polypeptide that
      (a) has the length selected in step (iii); and
      (b) comprises an amino acid sequence identified in step (iv) in the next highest proportion of subjects in the model population;
   (vi) optionally repeating step (iv) and further optionally step (v) in one or more further rounds, wherein a further fragment of the target polypeptide is identified in each round, and wherein in each round subjects are excluded from the model population if any of the fragments selected in step (iv) and not rejected in step (v) of any of the preceding rounds comprises an amino acid sequence identified in step (ii) for that subject; and
   (vii) designing or preparing a peptide, a polynucleic acid or vector that encodes a peptide, a panel of peptides, or one or more polynucleic acids or vectors that encode a panel of peptides, wherein each peptide comprises one or more of the target polypeptide fragments identified in step (iv), (v) or (vi), optionally wherein the polypeptide fragment is flanked at the N and/or C terminus by additional amino acids that are not part of the sequence of the target polypeptide antigen.
16. The method according to item 15, wherein the target polypeptide is expressed by pathogenic organism, a virus or a cancer cell, or is a cancer testes antigen, optionally wherein the target polypeptide is selected from the antigens listed in any of Tables 2 to 5.
17. The method according to item 15 or item 16, further comprising selecting two or more peptides, polynucleic acids or vectors designed or prepared according to the method of item 15 or 16 for use in a method of vaccinating, providing immunotherapy to, or inducing a cytotoxic and/or helper T cell response in a subject, optionally wherein each of the two or more peptides or encoded peptides comprises an amino acid sequence that is
   (a) a fragment of a polypeptide that is expressed by a pathogenic organism, a virus or a cancer cell; and
   (b) a T cell epitope capable of binding to at least three HLA class I molecules of the subject or a T cell epitope capable of binding to at least four HLA class II molecules of the subject;
   and wherein the method further comprises administering the one or more peptides, polynucleic acids or vectors to the subject.
18. A pharmaceutical composition comprising a panel of peptides, polynucleic acids or vectors designed and/or prepared according to the method of item 15 or item 16, or comprising or encoding two or more peptides designed and/or prepared according to the method of item 15 or item 16.
19. A pharmaceutical composition comprising a panel of peptides, or one or more polynucleic acids or vectors encoding a panel of peptides, for use in a method of inducing a T cell response against one or more target polypeptides in a subject of a target human population, wherein each of the peptides, or encoded peptides, comprises an amino acid sequence that is
   (a) 9 to 50 amino acids in length; and
   (b) comprises a fragment of the one or more target polypeptides, wherein the fragment comprises, in at least 10% of subjects of the intent-to-treat human population:
      a. an amino acid sequence of the target polypeptide that is a T cell epitope capable of binding to at least three HLA class I molecules of the subject;
      b. an amino acid sequence of the target polypeptide that is a T cell epitope capable of binding to at least four HLA class II molecules of the subject;
      c. an amino acid sequence of the target polypeptide that comprise a T cell epitope capable of binding to at least three HLA class I molecules of the subject and a T cell epitope capable of binding to at least four HLA class II molecules of the subject; or
      d. an amino acid sequence of the target polypeptide that both
         i. is a T cell epitope capable of binding to at least four HLA class II molecules; and
         ii. comprise an amino acid sequence that is a T cell epitope capable of binding to at least three HLA class I molecules of the subject
20. The pharmaceutical composition according to item 18 or item 19, further comprising a pharmaceutically acceptable adjuvant, diluent, carrier, preservative, or a combination thereof
21. A method of vaccination, providing immunotherapy or inducing a cytotoxic T cell response in a subject, the method comprising administering to the subject a pharmaceutical composition according to any of item 18 to item 20.
22. The method of item 21, wherein one or more or each of the peptides or the encoded peptides of the pharmaceutical composition comprises an amino acid sequence that is
   (a) a fragment of a polypeptide that is expressed by a pathogenic organism, a virus or a cancer cell; and
   (b) T cell epitope capable of binding to at least three HLA class I molecules of the subject or a T cell epitope capable of binding to at least four HLA class II molecules of the subject.
23. A method of providing immunotherapy to a subject in need thereof, the method comprising: administering to the individual a pharmaceutical composition, comprising i) two or more different peptides consisting of an amino acid sequence selected from the group consisting of SEQ ID Nos: 1 to 2786 and 5432 to 5931 and ii) a pharmaceutically acceptable adjuvant, diluent, carrier, preservative, or a combination thereof, thereby inducing an immune response.
24. The method of item 23, further comprising:
   predicting that the subject will have a CD8+ T cell response and/or a CD4+ T cell response to each peptide of the pharmaceutical composition by,
   (i) a) determining that each peptide comprises at least one amino acid sequence that is a T cell epitope capable of binding to at least three HLA class I molecules of the subject; and
      b) predicting that the subject will have a CD8+ T cell response to each peptide of the pharmaceutical composition or each peptide, polynucleic acid or vector of the kit; and
   (ii) a) determining that each peptide comprises at least one amino acid sequence that is a T cell epitope capable of binding to at least three HLA class II molecules of the subject; and
      b) predicting that the subject will have a CD4+ T cell response to each peptide of the pharmaceutical composition.
25. The method of item 23, wherein the pharmaceutical composition comprises at least 3, at least 4, at least 5, at least 6, at least 7, at least 8, at least 9, at least 10, at least 11, or at least 12 different peptides, wherein each peptide consists of an amino acid sequence selected from the group consisting of SEQ ID Nos: 1 to 2786 and 5432 to 5931.
26. The method of item 23, wherein the peptides of the pharmaceutical composition are from different cancer associated antigens selected from Table 24.
27. The method of item 23, wherein the adjuvant comprises an aluminium salt, saponin, Lipid A, or a water-in-oil emulsion.
28. The method according to item 23, wherein the immunotherapy is a treatment for cancer.
29. The method of item 28, wherein the cancer is bladder cancer, brain cancer, breast cancer, colorectal cancer, gastric cancer, hepatocellular cancer, leukemia, lung cancer, lymphoma, melanoma, ovarian cancer, pancreatic cancer, pediactric cancer, thyroid cancer, prostate cancer, kidney cancer, head and neck cancer, esophageal cancer and cervical cancer.
30. A pharmaceutical composition, comprising
   (a) at least two peptides consisting of 15 to 50 amino acids in length, wherein each peptide comprises a different sequence and which binds to at least three HLA class II alleles and at least three HLA class I alleles; and
   (b) an immunological adjuvant.
31. The pharmaceutical composition of item 30, wherein the at least two peptides each comprise a different sequence selected from SEQ ID Nos: 1 to 2786 and/or 5432 to 5931.
32. The pharmaceutical composition of item 30, comprising at least 3, at least 4, at least 5, at least 6, at least 7, at least 8, at least 9, at least 10, at least 11, or at least 12 different peptides, wherein each peptide comprises an amino acid sequence selected from the group consisting of SEQ ID Nos: 1 to 2786 and 5432 to 5931.
33. The pharmaceutical composition of item 30, wherein the peptides are from different cancer associated antigens selected from Table 24.
34. The pharmaceutical composition of item 30, wherein the adjuvant comprises an aluminium salt, saponin, Lipid A, or a water-in-oil emulsion.
35. A method of designing or preparing a peptide, or a polynucleic acid or vector that encodes a peptide, or a panel of peptides, or one or more polynucleic acid or vectors that encode a panel of peptides, for use in a method of inducing a T cell response against a target polypeptide, the method comprising
   (i) selecting or defining a model human population comprising a plurality of subjects each defined by HLA class I genotype and/or by HLA class II genotype;
   (ii) identifying for each subject of the model population:
      (a) amino acid sequences of the target polypeptide that are a T cell epitope capable of binding to at least three HLA class I molecules of the subject;
      (b) amino acid sequences of the target polypeptide that are a T cell epitope capable of binding to at least three HLA class II molecules of the subject;
      (c) amino acid sequences of the target polypeptide that comprise a T cell epitope capable of binding to at least three HLA class I molecules of the subject and a T cell epitope capable of binding to at least three HLA class II molecules of the subject; or
      (d) amino acid sequences of the target polypeptide that both
         a. are a T cell epitope capable of binding to at least three HLA class II molecules; and
         b. comprise an amino acid sequence that is a T cell epitope capable of binding to at least three HLA class I molecules of the subject;
   (iii) selecting a polypeptide fragment window length of between 9 and 50 amino acids;
   (iv) identifying a fragment of the target polypeptide that
      (a) has the length selected in step (iii); and
      (b) comprises an amino acid sequence identified in any one of step (ii) (a) to (d) in the highest proportion of subjects in the model population; and
   (v) designing or preparing a peptide, a polynucleic acid or vector that encodes a peptide, a panel of peptides, or one or more polynucleic acids or vectors that encode a panel of peptides, wherein each peptide comprises one or more of the target polypeptide fragments identified in step (iv).
36. The method of item 35, wherein the polypeptide fragment is flanked at the N and/or C terminus by additional amino acids that are not part of the sequence of the target polypeptide antigen.
37. The method of item 35, further comprising (v) testing the fragment identified in step (iv) against additional pre-defined criteria, rejecting the fragment if the further pre-defined criteria are not met, and repeating step (iv) to identify an alternative fragment of the target polypeptide that has the length selected in step (iii); and comprises an amino acid sequence identified in step (iv) in the next highest proportion of subjects in the model population.
38. The method of item 37, further comprising repeating step (iv) and step (v) in one or more further rounds, wherein a further fragment of the target polypeptide is identified in each round, and wherein in each round subjects are excluded from the model population if any of the fragments selected in step (iv) and not rejected in step (v) of any of the preceding rounds comprises an amino acid sequence identified in step (ii) for that subject.
39. The method according to item 35, wherein the target polypeptide is expressed by pathogenic organism, a virus or a cancer cell, or is a cancer testes antigen, optionally wherein the target polypeptide is selected from the antigens listed in any of Tables 2 to 5.
40. The method according to item 35, further comprising selecting two or more peptides, polynucleic acids or vectors designed or prepared according to the method of claim 9 for use in a method of vaccinating, providing immunotherapy to, or inducing a cytotoxic and/or helper T cell response in a subject, wherein each of the two or more peptides or encoded peptides comprises an amino acid sequence that is
   (a) a fragment of a polypeptide that is expressed by a pathogenic organism, a virus or a cancer cell; and
   (b) a T cell epitope capable of binding to at least three HLA class I molecules of the subject or a T cell epitope capable of binding to at least four HLA class II molecules of the subject;
   and wherein the method further comprises administering the one or more peptides, polynucleic acids or vectors to the subject.

### EXAMPLES

### Example 1—HLA-Epitope Binding Prediction Process and Validation

Predicted binding between particular HLA and epitopes (9 mer peptides) was based on the Immune Epitope Database tool for epitope prediction (www.iedb.org).

The HLA I-epitope binding prediction process was validated by comparison with HLA class I-epitope pairs determined by laboratory experiments. A dataset was compiled of HLA I-epitope pairs reported in peer reviewed publications or public immunological databases.

The rate of agreement with the experimentally determined dataset was determined (Table 6). The binding HLA I-epitope pairs of the dataset were correctly predicted with a 93% probability. Coincidentally the non-binding HLA I-epitope pairs were also correctly predicted with a 93% probability.

TABLE 6
Analytical specificity and sensitivity of the
HLA-epitope binding prediction process.

| | True epitopes | False epitopes |
|---|---|---|
| | (n = 327) | (n = 100) (Non- |
| HLA-epitope pairs | (Binder match) | binder match) |
| HIV | 91% (32) | 82% (14) |
| Viral | 100% (35) | 100% (11) |
| Tumor | 90% (172) | 94% (32) |
| Other (fungi, bacteria, etc.) | 100% (65) | 95% (36) |
| All | 93% (304) | 93% (93) |

The accuracy of the prediction of multiple HLA binding epitopes was also determined (Table 7). Based on the analytical specificity and sensitivity using the 93% probability for both true positive and true negative prediction and 7% (=100%-93%) probability for false positive and false negative prediction, the probability of the existence of a multiple HLA binding epitope in a person can be calculated. The probability of multiple HLA binding to an epitope shows the relationship between the number of HLAs binding an epitope and the expected minimum number of real binding. Per PEPI definition three is the expected minimum number of HLA to bind an epitope (bold).

TABLE 7
Accuracy of multiple HLA binding epitopes predictions.

| Expected | | | | | | | |
|---|---|---|---|---|---|---|---|
| minimum | | | | | | | |
| number of | | | | | | | |
| real HLA | Predicted number of HLAs binding to an epitope | | | | | | |
| binding | 0 | 1 | 2 | 3 | 4 | 5 | 6 |
| 1 | 35% | 95% | 100% | 100% | 100% | 100% | 100% |
| 2 | 6% | 29% | 90% | 99% | 100% | 100% | 100% |
| 3 | 1% | 4% | 22% | 84% | 98% | 100% | 100% |
| 4 | 0% | 0% | 2% | 16% | 78% | 96% | 99% |
| 5 | 0% | 0% | 0% | 1% | 10% | 71% | 94% |
| 6 | 0% | 0% | 0% | 0% | 0% | 5% | 65% |

The validated HLA-epitope binding prediction process was used to determine all HLA-epitope binding pairs described in the Examples below.

### Example 2—Epitope Presentation by Multiple HLA Predicts Cytotoxic T Lymphocyte (CTL) Response

This study investigates whether the presentation of one or more epitopes of a polypeptide antigen by one or more HLA class I molecule of an individual is predictive for a CTL response. The study was carried out by retrospective analysis of six clinical trials, conducted on 71 cancer patients and 9 HIV-infected patients (Table 8). Patients from these studies were treated with an HPV vaccine, three different NY-ESO-1 specific cancer vaccines, one HIV-1 vaccine and a CTLA-4 specific monoclonal antibody (Ipilimumab) that was shown to reactivate CTLs against NY-ESO-1 antigen in melanoma patients. All of these clinical trials measured antigen specific CD8+ CTL responses (immunogenicity) in the study subjects after vaccination. In some cases, correlation between CTL responses and clinical responses were reported.

No patient was excluded from the retrospective study for any reason other than data availability. The 157 patient datasets (Table 8) were randomized with a standard random number generator to create two independent cohorts for training and evaluation studies. In some cases, the cohorts contained multiple datasets from the same patient, resulting in a training cohort of 76 datasets from 48 patients and a test/validation cohort of 81 datasets from 51 patients.

TABLE 8
Summary of patient datasets

| | | | | | | Immunoassay | |
|---|---|---|---|---|---|---|---|
| | | | | | # Data sets | performed in | HLA |
| C1inical | | Target | | # | (#antigen × | the clinical | genotyping |
| trial | Immunotherapy | Antigen | Disease | Patients* | #patient) | trials** | method |
| 1 | VGX-3100 | HPV16-E6 | Cervical | 17/18 | 5 × 17 | IFN-γ | High |
| | | HPV16-E7 | cancer | | | ELISPOT | Resolution |
| | | HPV18-E6 | | | | | SBT |
| | | HPV18-E7 | | | | | |
| | | HPV16/18 | | | | | |
| 2 | HIVIS vaccine | HIV-1 | AIDS | 9/12 | 2 × 9 | IFN-γ | Low-Medium |
| | | Gag HIV- | | | | ELISPOT | Resolution |
| | | 1 RT | | | | | SSO |
| 3 | rNY-ESO-1 | NY-ESO-1 | Breast-and | 18/18 | 1 × 18 | In vitro and | High |
| | | | ovarian | | | Ex vivo | Resolution |
| | | | cancers, | | | IFN-γ | SBT |
| | | | melanoma | | | ELISPOT | |
| | | | and | | | | |
| | | | sarcoma | | | | |
| 4 | Ipilimumab | NY-ESO-1 | Metastatic | 19/20 | 1 × 19 | ICS after | Low to |
| | | | melanoma | | | T-cell | medium |
| | | | | | | stimulation | resolution |
| | | | | | | | typing, SSP |
| | | | | | | | of genomic |
| | | | | | | | DNA, high |
| | | | | | | | resolution |
| | | | | | | | sequencing |
| 5 | NY-ESO-1f | NY-ESO-1 | Esophageal-, | 10/10 | 1 × 10 | ICS after | SSO probing |
| | | (91-110) | non-small- | | | T-cell | and SSP of |
| | | | cell lung- | | | stimulation | genomic |
| | | | and gastric | | | | DNA |
| | | | cancer | | | | |
| 6 | NY-ESO-1 | NY-ESO-1 | Esophageal- | 7/9 | 1 × 7 | ICS after | SSO probing |
| | overlapping | (79-173) | and lung | | | T-cell | and SSP of |
| | peptides | | cancer, | | | stimulation | genomic |
| | | | malignant | | | DNA | |
| | | | melanoma | | | | |
| Total | 6 | 7 | | 80 | 157 | | |

The reported CD8+ T cell responses of the training dataset were compared with the HLA class I restriction profile of epitopes (9 mers) of the vaccine antigens. The antigen sequences and the HLA class I genotype of each patient were obtained from publicly available protein sequence databases or peer reviewed publications and the HLA I-epitope binding prediction process was blinded to patients' clinical CD8+ T cell response data where CD8+ T cells are IFN-γ producing CTL specific for vaccine peptides (9 mers). The number of epitopes from each antigen predicted to bind to at least 1 (PEPI1+), or at least 2 (PEPI2+), or at least 3 (PEPI3+), or at least 4 (PEPI4+), or at least 5 (PEPIS+), or all 6 (PEPI6) HLA class I molecules of each patient was determined and the number of HLA bound were used as classifiers for the reported CTL responses. The true positive rate (sensitivity) and true negative rate (specificity) were determined from the training dataset for each classifier (number of HLA bound) separately.

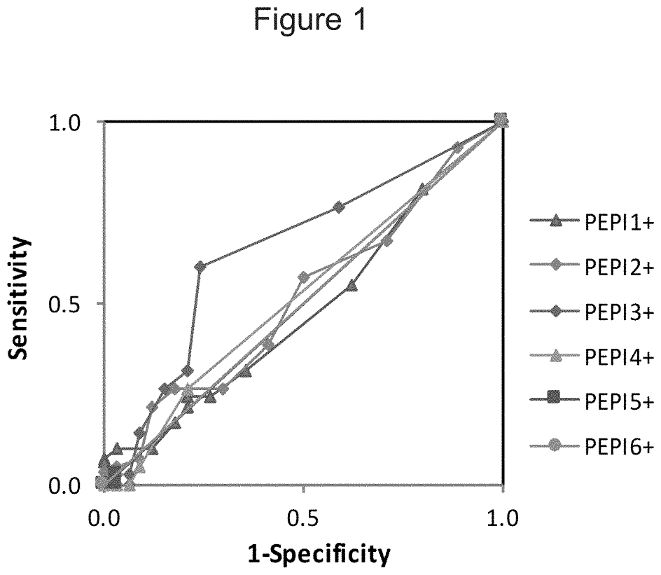

ROC analysis was performed for each classifier. In a ROC curve, the true positive rate (Sensitivity) was plotted in function of the false positive rate (1-Specificity) for different cut-off points (FIG. 1). Each point on the ROC curve represents a sensitivity/specificity pair corresponding to a particular decision threshold (epitope (PEPI) count). The area under the ROC curve (AUC) is a measure of how well the classifier can distinguish between two diagnostic groups (CTL responder or non-responder).

The analysis unexpectedly revealed that predicted epitope presentation by multiple class I HLAs of a subject (PEPI2+, PEPI3+, PEPI4+, PEPIS+, or PEPI6), was in every case a better predictor of the CD8+ T cell response or CTL response than epitope presentation by merely one or more HLA class I (PEPI1+, AUC=0.48, Table 9).

TABLE 9
Determination of diagnostic value of the
PEPI biomarker by ROC analysis

| Classifiers | AUC |
|---|---|
| PEPI1+ | 0.48 |
| PEPI2+ | 0.51 |
| PEPI3+ | 0.65 |
| PEPI4+ | 0.52 |
| PEPI5+ | 0.5 |
| PEPI6+ | 0.5 |

The CTL response of an individual was best predicted by considering the epitopes of an antigen that could be presented by at least 3 HLA class I alleles of an individual (PEPI3+, AUC=0.65, Table 9). The threshold count of PEPI3+ (number of antigen-specific epitopes presented by 3 or more HLA of an individual) that best predicted a positive CTL response was 1 (Table 10). In other words, at least one antigen-derived epitope is presented by at least 3 HLA class I of a subject (≥1 PEPI3+), then the antigen can trigger at least one CTL clone, and the subject is a likely CTL responder. Using the ≥1 PEPI3+ threshold to predict likely CTL responders (“≥1 PEPI3+ test”) provided 76% true positive rate (diagnostic sensitivity) (Table 10).

TABLE 10
Determination of the ≥1 PEPI3+ threshold to predict likely CTL
responders in the training dataset.

| | PEPI3+ Count | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Sensitivity: | 0.76 | 0.60 | 0.31 | 0.26 | 0.14 | 0.02 | 0 | 0 | 0 | 0 | 0 | 0 |
| 1-Specificity: | 0.59 | 0.24 | 0.21 | 0.15 | 0.09 | 0.06 | 0.06 | 0.03 | 0.03 | 0.03 | 0.03 | 0.03 |

### Example 3—Retrospective Validation of the ≥1 PEPI3+ Threshold as Novel Biomarker for PEPI Test

In a retrospective analysis, the test cohort of 81 datasets from 51 patients was used to validate the ≥1 PEPI3+ threshold to predict an antigen-specific CD8+ T cell response or CTL response. For each dataset in the test cohort it was determined whether the ≥1 PEPI3+ threshold was met (at least one antigen-derived epitope presented by at least three class I HLA of the individual). This was compared with the experimentally determined CD8+ T cell responses (CTL responses) reported from the clinical trials (Table 11).

The retrospective validation demonstrated that a PEPI3+ peptide induces CD8+ T cell response (CTL response) in an individual with 84% probability. 84% is the same value that was determined in the analytical validation of the PEPI3+ prediction, epitopes that binds to at least 3 HLAs of an individual (Table 7). These data provide strong evidences that immune responses are induced by PEPIs in individuals.

Table 11
Diagnostic performance characteristics of the ≥1 PEPI3+ test (n = 81).

| Performance | characteristic | Description | Result |
|---|---|---|---|
| Positive | 100% | The likelihood that an individual that meets the | 84% |
| predictive | [A/(A + B)] | ≥1 PEPI3+ threshold has antigen-specific CTL | |
| value (PPV) | | responses after treatment with immunotherapy. | |
| Sensitivity | 100% | The proportion of subjects with antigen-specific | 75% |
| | [A/(A+C)] | CTL responses after treatment with | |
| | | immunotherapy who meet the ≥1 PEPI3+ | |
| | | threshold. | |
| Specificity | 100% | The proportion of subjects without antigen- | 55% |
| | [D/B + D)] | specific CTL responses after treatment with | |
| | | immunotherapy who do not meet the ≥1 PEPI3+ | |
| | | threshold. | |
| Negative | 100% | The likelihood that an individual who does not | 42% |
| predictive | [D/(C + D)] | meet the ≥1 PEPI3+ threshold does not have | |
| value | | antigen-specific CTL responses after treatment | |
| (NPV) | | with immunotherapy. | |
| Overall | 100% | The percentage of predictions based on the ≥1 | 70% |
| percent | [(A + D)/ N] | PEPI3+ threshold that match the experimentally | |
| agreement | | determined result, whether positive or negative. | |
| (OPA) | | | |
| Fisher's exact (p) | | | 0.01 |

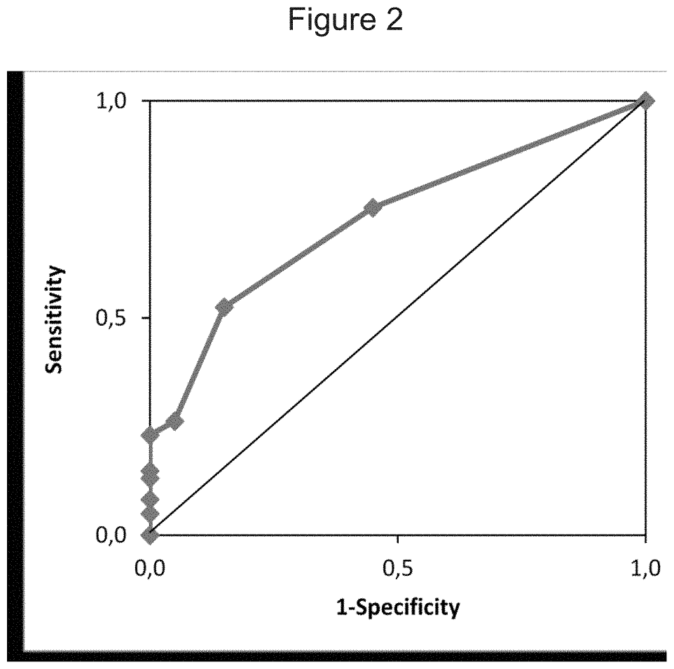

ROC analysis determined the diagnostic accuracy, using the PEPI3+ count as cut-off values (FIG. 2). The AUC value=0.73. For ROC analysis an AUC of 0.7 to 0.8 is generally considered as fair diagnostic value.

A PEPI3+ count of at least 1 (>1 PEPI3+) best predicted a CTL response in the test dataset (Table 12). This result confirmed the threshold determined during the training (Table 9).

TABLE 12
Confirmation of the ≥1 PEPI3+ threshold to predict likely CTL
responders in the test/validation dataset.

| | PEPI3+ Count | | | | | | | | | | | |
|---|---|---|---|---|---|---|---|---|---|---|---|---|
| | 1 | 2 | 3 | 4 | 5 | 6 | 7 | 8 | 9 | 10 | 11 | 12 |
| Sensitivity: | 0.75 | 0.52 | 0.26 | 0.23 | 0.15 | 0.13 | 0.08 | 0.05 | 0 | 0 | 0 | 0 |
| 1-Specificity: | 0.45 | 0.15 | 0.05 | 0 | 0 | 0 | 0 | 0 | 0 | 0 | 0 | 0 |

### Example 4—Clinical Validation of the ≥1 PEPI3+ Threshold as Novel Biomarker for PEPI Test

The PEPI3+ biomarker-based vaccine design has been tested first time in a phase I clinical trial in metastatic colorectal cancer (mCRC) patients in the OBERTO phase I/II clinical trial (NCT03391232). In this study, we evaluated the safety, tolerability and immunogenicity of a single or multiple dose(s) of PolyPEPI1018 as an add-on to maintenance therapy in subjects with mCRC. PolyPEPI1018 is a peptide vaccine containing 12 unique epitopes derived from 7 conserved TSAs frequently expressed in mCRC (WO2018158455 A1). These epitopes were designed to bind to at least three autologous HLA alleles that are more likely to induce T-cell responses than epitopes presented by a single HLA (See Examples 2 & 3). mCRC patients in the first line setting received the vaccine (dose: 0.2 mg/peptide) just after the transition to maintenance therapy with a fluoropyrimidine and bevacizumab. Vaccine-specific T-cell responses were first predicted by identification of PEPI3+-s in silico (using the patient's complete HLA genotype and antigen expression rate specifically for CRC) and then measured by ELISpot after one cycle of vaccination (phase I part of the trial).

Seventy datasets from 10 patients (Phase 1 cohort and dataset of OBERTO trial) was used to prospectively validate that PEPI3+ biomarker predicts antigen-specific CTL responses. For each dataset, predicted PEPI3+-s were determined in silico and compared to the vaccine-specific immune responses measured by ELISPOT assay from the patients' blood. Diagnostic characteristics (positive predictive value, negative predictive value, overall percent agreement) determined this way were then compared with the retrospective validation results described in Example 3.

The overall percent agreement was 64%, with high positive predictive value of 79%, representing 79% probability that the patient with predicted PEPI3+ will produce CD8 T cell specific immune response against the analyzed antigen. Clinical trial data were significantly correlated with the retrospective trial results (p=0.01) and provides evidence for the PEPI3+ calculation with PEPI test to predict antigen-specific T cell responses based on the complete HLA-genotype of patients (Table 13).

TABLE 13
Prospective validation of the ≥1 PEPI3+ and PEPI test

| | | | Prospective |
|---|---|---|---|
| | | Retrospective | validation |
| | | validation | (OBERTO) |
| Parameter | Definition | n = 81* | n = 70** |
| PPV | The likelihood that an individual with | 84% | 79% |
| Positive Predictive | a positive PEPI test result has antigen- | | |
| Value | specific T cell responses | | |
| NPV | The likelihood that an individual with | 42% | 51% |
| Negative Predictive | a negative PEPI test result does not | | |
| Value | have antigen-specific T cell responses | | |
| OPA | The percentage of results that are true | 70% | 64% |
| Overall Percent | results, whether positive or negative | | |
| Agreement | | | |
| Fisher's exact | | 0.01 | 0.01 |
| probability test (p) | | | |

*51 patients; 6 clinical trials; 81 dataset
**10 patients; Treos phase I clinical trial (OBERTO); 70 datasets

### Example 5—the ≥1 PEPI3+ Test Predicts CD8+ T Cell Reactivities

Supporting data were obtained to show that the ≥1 PEPI3+ correlates with clinical immunogenicity data but the state-of-art mono-HLA specific epitope determination does not show correlation with vaccine-specific immunogenicity.

The ≥1 PEPI3+ calculation was compared with a state-of-art method for predicting a specific human subject's CTL response to peptide antigens.

The HLA genotypes of 28 cervical cancer and VIN-3 patients that received HPV-16 specific synthetic long peptide vaccine (LPV) in two different clinical trials were determined from DNA samples. The LPV consists of long peptides covering the HPV-16 viral oncoproteins E6 and E7. The amino acid sequence of the LPV was obtained from M. J. Welters, et al. Induction of tumor-specific CD4+ and CD8+ T-cell immunity in cervical cancer patients by a human papillomavirus type 16 E6 and E7 long peptides vaccine. Clin Cancer Res 14, 178-187 (2008)., G. G. Kenter, et al. Vaccination against HPV-16 oncoproteins for vulvar intraepithelial neoplasia. N Engl J Med 361, 1838-1847 (2009). M. J. Welters, et al. Success or failure of vaccination for HPV16-positive vulvar lesions correlates with kinetics and phenotype of induced T-cell responses. Proc Natl Acad Sci USA 107, 11895-11899 (2010). The publications also report the T cell responses of each vaccinated patient to pools of overlapping peptides of the vaccine. 25 (20 having VIN-3 and 5 having cervical cancer) patients had immune response data available, and 25 had clinical response data available.

For each patient, epitopes (9 mers) of the LPV that are presented by at least three patient class I HLA (PEPI3+s) were identified and their distribution among the peptide pools was determined. Peptides that comprised at least one PEPI3+ (≥1 PEPI3+) were predicted to induce a CD8+ T cell response. Peptides that comprised no PEPI3+ were predicted not to induce a CD8+ T cell response.

The ≥1 PEPI3+ threshold correctly predicted 529 out of 555 negative CD8+ T cell responses (95% true negative (TN) rate) and 9 out of 45 positive CD8+ T cell responses (20% true positive (TP) rate) measured after vaccination (FIG. 3A). Overall, the agreement between the ≥1 PEPI3+ threshold and experimentally determined CD8+ T cell reactivity was 90% (p<0.001). For each patient the distribution among the peptide pools of epitopes that are presented by at least one patient class I HLA (≥1 PEPI1+, HLA restricted epitope prediction, prior art method) was also determined. Forty-two HLA class I binding epitopes predicted 45 CD8+ T cell responses (93% TP rate). In contrast, of the 555 negative T cell responses, only 105 were ruled out by HLA binding epitopes (19% TN rate) (FIG. 3B). Overall, the agreement between a single HLA class I allele binding epitope and CD8+ T cell response was 25%, which was not statistically significant.

### Example 6—Prediction of HLA Class II Restricted CD4+ Helper T Cell Epitopes

The 28 cervical cancer and VIN-3 patients that received the HPV-16 synthetic long peptide vaccine (LPV) in two different clinical trials (as detailed in Example 5) were investigated for CD4+ T helper responses following LPV vaccination (FIGS. 4A-B). The TP rate of the prediction of HLA class II restricted epitopes was 95%, since the State of Art tool predicted 112 positive responses (positive CD4+ T cell reactivity to a peptide pool for a person's HLA class II alleles) out of 117. The TN rate was 0% since it could rule out 0 of 33 negative T cell responses. Overall, the agreement between HLA-restricted class II PEPI prediction and CD4+ T cell reactivity was 75% (not significant).

The HLA class II-binding PEPI3+-s predicted 86 of 117 positive CD4+ T-cell responses (73% TP rate) and ruled out 17 of 33 negative T-cell responses (52% TN rate). Overall, the agreement between HLA class II PEPI3+-s and CD4+ T-cell response was 69% (p=0.005) (FIG. 4A).

### Example 7—The ≥1 PEPI3+ Test Predicts T Cell Responses to Full Length LPV Polypeptides

Using the same studies as reported in Examples 5 and 6, the ≥1 PEPI3+ test was used to predict patient CD8+ and CD4+ T cell responses to the full length E6 and E7 polypeptide antigens of the LPV vaccine. Results were compared to the experimentally determined responses reported. The test correctly predicted the CD8+ T cell reactivity (PEPI3+) of 11 out of 15 VIN-3 patients with positive CD8+ T cell reactivity test results (sensitivity 70%, PPV 85%) and of 2 out of 5 cervical cancer patients (sensitivity 40%, PPV 100%) (FIG. 5A). The CD4+ T cell reactivities (PEPI3+) were correctly predicted 100% both of VIN-3 and cervical cancer patients (FIG. 5B). Class I and class II HLA restricted PEPI3+ count was also observed to correlate with the reported clinical benefit to LPV vaccinated patients. Patients with higher PEPI3+ counts had either complete or partial response already after 3 months. There was also a correlation between the number of PEPIs and clinical response in VIN-3 patients for HLA class II PEPIs but not HLA class I PEPIs, confirming the post-hoc analysis results from the clinical trial (FIGS. 5C and 5D).

### Example 8—Case Study, PEPI3+ Correlation with Vaccine-Specific Immunogenicity

“Vaccine-1” is an HPV16 based DNA vaccine containing full length E6 and E7 antigens with a linker in between. “Vaccine-2” is an HPV18 based DNA vaccine containing full length E6 and E7 antigens with a linker in between (FIG. 6A). A Phase II clinical trial investigated the T cell responses of 17 HPV-infected patients with cervical cancer who were vaccinated with both “Vaccine-1” and “Vaccine-2” (“Vaccine-3” vaccination, Bagarazzi et al. Science Translational Medicine. 2012; 4(155):155ra138.).

FIG. 6B shows for two illustrative patients (patient 12-11 and patient 14-5) the position of each epitope (9 mer) presented by at least 1 (PEPI1+), at least 2 (PEPI2+), at least 3 (PEPI3+), at least 4 (PEPI4+), at least 5 (PEPIS+), or all 6 (PEPI6) class I HLA of these patients within the full length sequence of the two HPV-16 and two HPV-18 antigens.

Patient 12-11 had an overall PEPI1+ count of 54 for the combined vaccines (54 epitopes presented by one or more class I HLA). Patient 14-5 had a PEPI1+ count of 91. Therefore, patient 14-5 has a higher PEPI1+ count than patient 12-11 with respect to the four HPV antigens. The PEPI1+s represent the distinct vaccine antigen specific HLA restricted epitope sets of patients 12-11 and 14-5. Only 27 PEPI1+s were common between these two patients. For the PEPI3+ counts (number of epitopes presented by three or more patient class I HLA), the results for patients 12-11 and 14-5 were reversed. Patient 12-11 had a PEPI3+ count of 8, including at least one PEPI3+ in each of the four HPV16/18 antigens. Patient 14-5 had a PEPI3+ count of 0 (FIG. 6C).

The reported immune responses of these two patients matched the PEPI3+ counts, not the PEPI1+ counts. Patient 12-11 developed immune responses to each of the four antigens post-vaccination as measured by ELISpot, whilst patient 14-5 did not develop immune responses to any of the four antigens of the vaccines. A similar pattern was observed when the PEPI1+ and PEPI3+ sets of all 17 patients in the trial were compared. There was no correlation between the PEPI1+ count and the experimentally determined T cell responses reported from the clinical trial. However, correlation between the T cell immunity predicted by the ≥1 PEPI3+ test and the reported T cell immunity was observed. The ≥1 PEPI3+ test predicted the immune responders to HPV DNA vaccine.

Moreover, the diversity of the patient's PEPI3+ set resembled the diversity of T cell responses generally found in cancer vaccine trials. Patients 12-3 and 12-6, similar to patient 14-5, did not have PEPI3+s predicting that the HPV vaccine could not trigger T cell immunity. All other patients had at least one PEPI3 predicting the likelihood that the HPV vaccine can trigger T cell immunity. 11 patients had multiple PEPI3+ predicting that the HPV vaccine likely triggers polyclonal T cell responses. Patients 15-2 and 15-3 could mount high magnitude T cell immunity to E6 of both HPV, but poor immunity to E7. Other patients 15-1 and 12-11 had the same magnitude response to E7 of HPV18 and HPV16, respectively.

### Example 9—Design of a Model Population for Conducting in Silico Trials and Identifying Candidate Precision Vaccine Targets for Large Population

An in silico human trial cohort of 433 subjects with complete 4-digit HLA class I genotype (2×HLA-A*xx:xx; 2×HLA-B*xx:xx; 2×HLA-C*xx:xx) and demographic information was compiled. This Model Population has subjects with mixed ethnicity having a total of 152 different HLA alleles that are representative for >85% of presently known allele G-groups.

A database of a “Big Population” containing 7,189 subjects characterized with 4-digit HLA genotype and demographic information was also established. The Big Population has 328 different HLA class I alleles. The HLA allele distribution of the Model Population significantly correlated with the Big Population (Table 14) (Pearson p<0.001). Therefore, the 433 patient Model Population is representative for a 16 times larger population. The Model Population is representative for 85% of the human race as given by HLA diversity as well as HLA frequency.

TABLE 14
Statistical analysis of HLA distributions in “Model Population” vs. “Big Population”.

| Group name 1 | Group name 2 | Pearson R value | Correlation | P Value |
|---|---|---|---|---|
| 433 Model | 7,189 Big Population | 0.89 | Strong | P < 0.001 |
| Population | | | | |

### Example 10—in Silico Trial Based on the Identification of Multiple HLA Binding Epitopes in a Multi-Peptide Vaccine IMA901 Predict the Reported Clinical Trial Immune Response Rate

### Probability of Targeting Multiple Antigens in the Tumor of RCC Patients

IMA901 is a therapeutic vaccine for renal cell cancer (RCC) comprising 9 peptides derived from tumor-associated antigens (TUMAPs). It was demonstrated that TUMAPs are naturally presented in human cancer tissue, they are overexpressed antigens shared by a subset of patients with the given cancer entity (Table 15). We estimated the probability that a TSA is expressed in a subject treated with IMA901 vaccine using available data from the scientific literature (FIG. 7). We used the Bayesian convention assuming that the expression probabilities follow a Beta-distribution.

We defined AG50 as the number of TSAs (AG) in the cancer vaccine that a specific tumor type expresses with 50% probability. The AG50 modelling of cancer vaccines assumes that each AG produces an effect proportional to the expression rate of the AG in the tumor type (if each AG in the vaccine is immunogenic).

For IMA901 vaccine targeting 9 antigens (9 TUMAPs), the AG50 value is 4.7, meaning that about half of the antigens are overexpressed in 50% of patients' tumor. Moreover, the probability of targeting 2 expressed antigens is 100% and 3 antigens is 96%. These results suggest high potency of IMA901 vaccine based on target antigen selection.

TABLE 15
Overexpression of TAAs in RCC tumors selected for IMA901 vaccine

| | Published expression rate in | | |
|---|---|---|---|
| TAA (AG) | RCC tumors* | Estimated expression rate (95% CI) | |
| ADF-001 | 5/11¹ | 46% | (21%, 72%) |
| ADF-002 | 5/11 | 46% | (21%, 72%) |
| APO-001 | 9/11² | 77% | (52%, 95%) |
| CCN-001 | 4/11 | 38% | (15%, 65%) |
| GUC-001 | 0/2² | 25% | (1%, 71%) |
| K67-001 | 2/2 | 75% | (29%, 99%) |
| MET-001 | 11/11 | 92% | (74%, 100%) |
| MUC-001 | 0/11 | 8% | (0%, 26%) |
| RGS-001 | 7/11 | 62% | (35%, 85%) |

*expression is defined as overexpression in tumors compared to healthy tissues provided in the source publications
¹Walter S et al, Multipeptide immune response to cancer vaccine IMA901 after single-dose cyclophosphamide associates with longer patient survival, Nature Medicine, (2012), 18, 1254-1261
²Krüger T et al, Lessons to be learned from primary renal cell carcinomas, Cancer Immunol, Immunother, 2005, 54, 826-836

### Probability of Inducing Immune Responses Against Multiple Antigens in the Tumor of RCC Patients

A total of 96 HLA-A*02+ subjects with advanced RCC were treated with IMA901 in two independent clinical studies (Phase I and Phase II) (Walter S et al, Multipeptide immune response to cancer vaccine IMA901 after single-dose cyclophosphamide associates with longer patient survival, Nature Medicine, (2012), 18, 1254-1261). Each of the 9 peptides in IMA901 have been identified as HLA-A*02-restricted epitopes. Based on currently accepted standards, all 9 peptides are strong candidates to boost T cell responses against renal cancer since their presence has been detected in renal cancer patients, and because the trial patients were specifically selected to have at least one HLA molecule (HLA-A*02) capable of presenting each of the peptides. Despite this restriction the immune response rate of the phase I and phase II clinical trials measured for at least one peptide of the vaccine was 74% and 64%, respectively. We analyzed by in silico prediction the HLA binding properties of each TUMAP in IMA901 and found that 8 out of the 9 TUMAPs can bind to many HLA-A*02 alleles confirming the identification process (FIGS. 8A-B). However, we found that each TUMAP can bind to many other HLA-B* and HLA-C* alleles (FIG. 8A).

Since the complete 4-digit HLA genotype of subjects who participated in IMA901 clinical trials were not available, we used the genotype data of 51 HLA-A*02 selected RCC subjects from another clinical trial, to characterize the immunogenicity of IMA901 vaccine (REF: Chowell D, Morris L G T, Grigg C M, Weber J K, Samstein R M, et al. Patient HLA class I genotype influences cancer response to checkpoint blockade immunotherapy. Science. 2018; 359 (6375): 582-587.). As presented on FIG. 8B, only few TUMAPs are able to bind to multiple HLAs of the same subject. The most immunogenic peptide in this context turned to be MET-001 capable of generating PEPI in 35% of RCC patients. However, CCN-001 could not generate PEPI in any of the patients, in agreement with FIG. 8A; CCN-001 can bind only to HLA-A*02 alleles. Based on FIG. 8A, MUC-001 is theoretically able to bind other alleles, too (both HLA-B and HLA-C), however those alleles were not present in the patients of our model population, therefore this peptide could not generate PEPI, either.

The immunogenicity of IMA901 vaccine determined in the 2 clinical trials was compared with the PEPI response rate determined using the PEPI test in our RCC model population. We found 67% (CI95 53-78%) immune response to at least one peptide of the IMA901 vaccine. According to PEPI test, 33% (CI95 22-47%) of these HLA-A*02+ subjects did not have 3 HLAs binding to any TUMAPs. Interestingly, IMA901 did not induce T cell responses in 25% and 36% of HLA-A*02 selected subjects in the Phase I and Phase II clinical trials, respectively. Furthermore, PEPI test predicted 30% (CI95 19-43%) of subjects with 1 PEPI to one TUMAP, and 37% (CI95 25-51%) have ≥2 PEPIs to at least two IMA901 peptides, which is in agreement with the average 40% and 27% immune response to 1 or ≥2 TUMAPs in both clinical trials (Table 16). The differences between the immunogenicity found in the 3 cohorts can be explained by the differences in the HLA genotype of the study subjects as well as the potential errors in measuring T cell responses and in determining PEPIs with the PEPI test (see Example 1). The phase I and phase II study results show the variability of the immune response rates of the same vaccine in different trial cohorts. However, the agreements between PEPI response rates and immunogenicity of peptide vaccines are determined by the host HLA sequences.

TABLE 16
Immunogenicity of IMA901 vaccine is determined
by the host HLA genotype (multiple HLAs)

| | | | | RCC model population | |
|---|---|---|---|---|---|
| Immune responses | Phase I | Phase II | Phase I + II | (n = 51)** | |
| to TUMAPs | (n = 27)* | (n = 61)* | (n = 88) | (CI95%) | |
| No peptide | 25% | 36% | 33% | 33% | (22-47%) |
| ≥1 peptide | 74% | 64% | 67% | 67% | (53-78%) |
| 1 peptide | 44% | 38% | 40% | 30% | (19-43%) |
| ≥2 peptides | 29% | 26% | 27% | 37% | (25-51%) |

*reported immunological data for the trials conducted with IMA901vaccine (REF: Walter Nat Med 2012);
**Predicted by PEPI test

Similarly to the AG50, we defined AP50 as the average number of antigens with PEPI of a vaccine which shows how the vaccine can induce immune response against the antigens targeted by the composition (cancer vaccine specific immune response). AP, therefore is depending of the HLA heterogeneity of the analyzed population and is independent on the expression of the antigen on the tumor. The IMA901 composition can induce immune response against an average of 1.06 vaccine antigens (AP50=1.06) meaning that in the HLA-A*02 selected RCC model population it can induce immune response against at least one vaccine antigen. This result is far less compared to the designed intention of immunogenicity (HLA-matched patients treated with 9 peptides).

### Comparison of Immunogenicity and Clinical Response of TUMAPs in the IMA901 Peptide Vaccine

An immune response induced by a vaccine against a single antigen might not be sufficient for clinical activity, as the given antigen might not be expressed in the patient. Therefore, we defined AGP as the immune response which targets an expressed antigen, taking into account both the immunogenicity and expression probability of the vaccine antigen on the tumor, presented above. AGP depends on the antigen (AG) expression rate in the indicated tumor and the HLA genotype of subjects capable to make PEPI (P) in the study population.

Therefore, we investigated the correlation between immune responses against different number of antigens (TUMAPs) and the immune responses against likely expressed antigens (AGP). We found that an immune response elicited by one peptide (1 TUMAP) corresponds to 0.98 AGP, meaning that there is 98% probability that the immune response induced by any peptide of the IMA901 vaccine will target an expressed antigen on the tumor (FIG. 9). However, immune responses elicited by 2 or 3 TUMAPs correspond to only 1.44 and 2.21 AGP, respectively. 0.35 AGP corresponding to 0 TUMAP indicates the cumulated error of PEPI test prediction (see Example 1).
characterize the potency of a cancer vaccine we defined AGP50, a parameter showing the number of antigens that the vaccine induced CTLs can recognize in a tumor with 50% probability. The computation is similar to the AG50 but in addition to the expression, the occurrence of the PEPI presentation on certain vaccine antigen is also considered. AGP50 for IMA901 vaccine for the RCC model population is 1.10.

In a retrospective analysis, IMA901 clinical trial investigators found that significantly more subjects who responded to multiple TUMAPs of IMA901 experienced disease control (DC, stable disease or partial response) compared with subjects who had no response or responded to only 1 TUMAP (Table 17). Since the presence of PEPIs accurately predicted the responders to TUMAPs, we investigated the relationship between disease control rate in the TUMAP responder subpopulation and AGP. Similarly, to the investigators we analyzed the percentage of patients who are likely to have immune response against an expressed antigen (i.e.: >1 AGP) for the subpopulations predicted to have immune response to 0, 1 or 2 TUMAPs using our RCC model population. Interestingly, percentage of patients with 1 AGP is similar to the percentage of patients with disease control in the subpopulations: i.e.: 33% of patients had disease control vs 47% (CI95 23-67%) had 1 AGP and considerably more patients had disease control and AGP in the subgroup with immune response to 2 TUMAPs 75% vs 90% (CI95 70, 97%), respectively. These results suggest that only those patients are likely to experience clinical benefit, who have immune response against at least one expressed tumor antigen. Moreover, the percentage of patients with 1 AGP in our RCC model population is similar to the disease control rate of the phase I and phase II trials conducted with IMA901 vaccine (Table 17).

TABLE 17
Correlation between clinical benefit and AGP

| | % of pts with DC | % pts with 1 AGP in the | |
|---|---|---|---|
| Subpopulation | in the clinical subpopulation | model subpopulation (CI95) | |
| No IR | 14% | 5% | (0%, 18%) |
| IR to 1TUMAP | 33% | 47% | (23%, 67%) |
| IR to ≥2 TUMAPs | 75% | 90% | (70%, 97%) |
| Phase I | 40% | 49% | (35%, 61%) |

| | |
|---|---|
| Phase II | 31% |

### Analysis of IMA901 Vaccine Potency in Multiple Populations

As shown in Table 18, AG50 value of 4.7 was observed for IMA901 vaccine, suggesting high potency based on target antigen selection. However, AP50 for IMA901 in both the unselected general population and HLA-A*02 selected subjects were only 0.75 and 1.12, respectively. Similar results were obtained for unselected RCC model population and HLA-A*02 selected populations. This results demonstrate that HLA-A*02 enrichment improved the antigenicity of IMA901, however did not ensure the immunogenicity of the vaccine. Consequently, the AGP50 values describing the potency of the vaccine are low in each population.

TABLE 18
Potency of IMA901 vaccine in in unselected
population and HLA-A*02 selected subjects

| Model Population | AG50 | AP50 | AGP50 |
|---|---|---|---|
| All Subjects (n = 433) | 4.7 | 0.75 | 0.49 |
| HLA-A*02 Subjects (n = 180) | 4.7 | 1.12 | 0.81 |
| RCC population (n = 129) | 4.7 | 0.61 | 0.70 |
| RCC subpopulation A*02 (n = 51) | 4.7 | 1.06 | 1.10 |

### Example 11—in Silico Trials Based on the Identification of Multiple HLA Binding Epitopes Predict the Reported T Cell Response Rates of Clinical Trials

The objective of this study was to determine whether a model population, such as the one described in Example 9, may be used to predict CTL reactivity rates of vaccines, i.e. used in an in silico efficacy trial and to determine the correlation between the clinical outcome of vaccine trials and PEPI.

Published clinical trial results were collected from studies with therapeutic vaccines, which included 1,790 subjects in 64 clinical studies, treated with 42 therapeutic vaccines covering 61 different antigens (Table 19). The same vaccines used in those clinical trials were used to perform in silico trials with the model population of 433 human leukocyte antigen (HLA)-genotyped subjects (described in Example 9). No subjects were excluded for reasons other than data availability. IRR was defined as the proportion of subjects in the study population with T cell responses induced by the study vaccine. ORR was defined as the proportion of subjects in the study population with objective response (complete and partial response) after vaccination. The proportion of subjects with PEPIs (personal epitopes that bind to 3 HLA alleles of a subject), multiple PEPIs, and PEPIs in multiple antigens were computed in the in silico trials to obtain the PEPI Score, MultiPEPI Score, and MultiAgPEPI Score, respectively. The immune and objective response rates (IRR and ORR) from the published clinical trials were compared with the PEPI Score, MultiPEPI Score, and MultiAgPEPI Score. All reported and calculated scores are summarized in Table 20.

TABLE 19
Summary of patient demographics in the published clinical trials

| Characteristic | Count | Percentage |
|---|---|---|
| Total subjects | 1,790 | |
| Total studies | 64 | |
| Subjects with HIV infection | 12 | 1% |
| Subjects with neoplasia or dysplasia | 172 | 9% |
| Subjects with cancer | 1606 | 90% |
| Subjects with solid tumors | 1503 | 84% |
| Subjects with liquid tumors | 103 | 6% |
| Subjects with metastatic tumors | 788 | 44% |
| Subjects with non-metastatic tumors | 818 | 46% |
| HLA selected subjects | 918 | 51% |
| Non-HLA selected subjects | 872 | 49% |
| Trials with HLA selected subjects | 32 | 50% |
| Trials without HLA selected subjects | 32 | 50% |

TABLE 20
Response rates and PEPI Scores

| | | | | Multi | MultiAg |
|---|---|---|---|---|---|
| | | | PEPI | PEPI | PEPI |
| Immunotherapy | IRR | ORR | Score | Score | Score |
| PSMA-Survivin pulsed DC vaccine | — | 18% | 3% | 0% | 0% |
| Peptide vaccine | — | 3% | 10% | 0% | 0% |
| HPV-SLP | 83% | 60% | 73% | 70% | 34% |
| | 100% | 60% | 73% | 70% | 34% |
| VGX-3100 | 78% | 50% | 87% | 56% | 64% |
| Melanoma peptide vaccine | 52% | 12% | 42% | 6% | 6% |
| GAA peptides vaccine | 55% | 15% | 18% | 0% | 0% |
| KRM-20 peptide vaccine | 40% | 13% | 36% | 15% | 15% |
| Peptide vaccine | 100% | 25% | 81% | 3% | 1% |
| S-288310 peptide vaccine | 67% | 17% | 44% | 8% | 8% |
| KIF20A-66 peptide | 70% | 26% | 38% | 7% | — |
| PepCan | 65% | 52% | 62% | 26% | — |
| Iplilimumab (NYESO-1 specific response) | 72% | 25% | 84% | 65% | — |
| p53 SLP70-248 | 88% | 0% | 77% | 52% | — |
| | 100% | 0% | 77% | 52% | — |
| | 0% | — | 77% | 52% | — |
| p53 SLP70-235 | 21% | — | 75% | 52% | — |
| GVX301 | 64% | 0% | 65% | 7% | — |
| TroVax vaccine (OXB-301) | 65% | 0% | 94% | 83% | — |
| | 57% | 0% | 94% | 83% | — |
| StimuVax | 21% | — | 2% | — | — |
| IMA901 | 74% | — | 48% | 27% | 27% |
| | 64% | — | 48% | 27% | 27% |
| ICT107 | 33% | — | 52% | — | — |
| ProstVac | 67% | — | 50% | 23% | — |
| | 45% | — | 50% | 23% | — |
| | 76% | — | 50% | 23% | — |
| | 67% | — | 50% | 23% | — |
| | 50% | — | 50% | 23% | — |
| | 72% | — | 50% | 23% | — |
| Synchrotope TA2M | 46% | — | 24% | 7% | — |
| MELITAC 12.1 | 49% | — | 47% | 19% | — |
| HIVIS | 50% | — | 88% | — | — |
| | 80% | — | 93% | — | — |
| ImMucin | 90% | — | 95% | 70% | — |
| | 100% | 47% | 95% | 70% | — |
| NY-ESO-1 OLP | 71% | — | 84% | 65% | — |
| | 82% | 0% | 84% | 65% | — |
| WT1 vaccine | 83% | — | 80% | 77% | — |
| WT1 peptide vaccine | 72% | 6% | 86% | — | — |
| RHAMM-R3 peptide vaccine | 44% | 0% | 0% | — | — |
| GMMG-MM5 peptides | 35% | — | 86% | 21% | 21% |
| INGN-225 p53 vaccine | 58% | 4% | 82% | 61% | — |
| HR2822 | 8% | — | 3% | — | — |
| GV1001 | 17% | — | 3% | — | — |
| | 45% | — | 3% | — | — |
| Vx-001 | 51% | — | 33% | — | — |
| | 66% | 7% | 33% | — | — |
| | 58% | 4% | 33% | — | — |
| | 71% | 0% | 33% | — | — |
| NY-ESO-1f | 90% | 0% | 55% | 18% | — |
| GL-0817 (MAGE-A3 Trojan) | 33% | — | 29% | 3% | — |
| | 57% | 0% | 29% | 3% | — |
| DPX0907 (per peptide) | 0% | — | 22% | — | — |
| | 11% | — | 18% | — | — |
| | 11% | — | 7% | — | — |
| | 11% | — | 39% | — | — |
| | 17% | — | 12% | — | — |
| | 17% | — | 5% | — | — |
| | 22% | — | 31% | — | — |
| CV9103 mRNA vaccine | 80% | — | 100% | — | — |
| TG4010 vaccine | 38% | 13% | 43% | 6% | — |
| | 26% | 0% | 43% | 6% | — |
| | 21% | — | 43% | 6% | — |
| | — | 0% | — | — | — |
| SVN-2B peptide vaccine | 60% | — | 35% | — | — |
| TSPP peptide vaccine | — | 5% | 72% | 31% | — |
| Her2/neu peptide vaccine (p369) | 62% | — | 4% | — | — |
| Her2/neu peptide vaccine (p688) | 31% | — | 1% | — | — |
| Her2/neu peptide vaccine (p971) | 54% | — | 0% | — | — |
| MART-1 Peptide Vaccine | 15% | — | 0% | — | — |

We investigated the correlation between ≥1 PEPI3+ Score and immune response rate in a previous study of 12 peptide vaccines derived from cancer antigens that induced T cell responses in a subpopulation of 172 subjects from 19 clinical trials, that were identified from peer reviewed publications. The experimentally determined response rates reported from the trials were compared with the ≥1 PEPI3+ Scores and linear correlation between ≥1 PEPI3+ Score and response rate (R²=0.70) was found (p=0.001) (FIG. 10A). The correlation between ≥1 PEPI3+ Score and immune response rate was then confirmed by the analysis of 59 clinical trials involving 1,343 subjects who were treated with 40 different vaccines. Each vaccine was analyzed by comparing the published IRR from the clinical trial to the PEPI Score from the model population (FIG. 10B). The correlation between the IRR and PEPI Score was significant (r²=0.465 and p=0.001). This result demonstrated that the PEPI Score determined by in silico trials in the MP accurately predicts the IRRs observed in clinical trials.

To test whether polyclonal T cell response increases the likelihood of tumor shrinkage, ORR and MultiPEPI Score were compared. Preliminary experiments analyzed the relationship between clinical response (either ORR or DCR) and MultiPEPI Score in 17 clinical trials conducted with peptide- and DNA-based immunotherapy vaccines. The results from these experiments demonstrated a significant correlation between clinical response rate and MultiPEPI Score (r²=0.75, p<0.001). To confirm these findings, ORR data from 27 clinical trials with 21 different vaccines, involving 600 subjects, were collected and analyzed (FIG. 10C). The MultiPEPI Score was calculated as the percentage of subjects in the model population with multiple PEPIs from the study vaccine. The results from this experiment demonstrated that ORR does not correlate with MultiPEPI Score (FIG. 10D).

Results from previous studies suggested that T cell responses against multiple antigens were associated with longer progression free- and overall survival. Consequently, we hypothesized that the induction of T cell responses against multiple tumor antigens increases the likelihood of tumor shrinkage. To test this hypothesis, ORR data from 10 clinical trials conducted with 9 different vaccines, involving 263 subjects, that were treated with multiantigen-targeting vaccine were collected and analyzed. The MultiAg PEPI Score was calculated as the percentage of subjects with vaccine-specific PEPIs on at least two antigens. The results from this experiment demonstrated a significant correlation between ORR and MultiAg PEPI Score (r²=0.64; p=0.01), and ORR and MultiPEPI Score (r²=0.88 and p=0.001) (FIGS. 10E and F, respectively). These results suggest that T cell responses against multiple tumor antigens may recognize a larger tumor cell population, thereby increasing the likelihood of tumor shrinkage.

The next analysis explored whether PEPI-specific T cell responses against antigens expressed in the tumor of interest, increase the likelihood of tumor shrinkage. A total of 15 clinical trials enrolled subjects with target antigen positive disease and 11 clinical trials had no subject preselection based on antigen expression. The proportion of subjects with objective response was significantly higher in CTs with target antigen-positive subjects compared with CTs without pre-selection (21.0% vs. 3.6%, respectively, p=0.03)

The correlation between ORR and MultiPEPI Score was statistically significant in subjects with confirmed expression of target antigens (r²=0.56, p=0.005) (FIG. 10G). These results emphasize the importance of the presence of cognate PEPI in the tumor, and also that the presence of the cognate PEPI in the tumor increases the likelihood of tumor shrinkage.

This study demonstrated that the link between a subject's HLA genotype and PEPI is the most important factor in predicting clinical response to a vaccine. This study also showed that the PEPI Score can predict the clinical outcome of therapeutic vaccines.

### Example 12—Study Design of OBERTO Phase I/II Clinical Trial and Preliminary Safety Data

OBERTO trial is a Phase I/II tria of PolyPEPI1018 Vaccine and CDx for the Treatment of Metastatic Colorectal Cancer (NCT03391232). Study design is shown on FIG. 11.

### Enrollment Criteria

Histologically confirmed metastatic adenocarcinoma originating from the colon or the rectum
Presence of at least 1 measurable reference lesion according to RECIST 1.1
PR or stable disease during first-line treatment with a systemic chemotherapy regimen and 1 biological therapy regimen
Maintenance therapy with a fluoropyrimidine (5-fluorouracil or capecitabine) plus the same biologic agent (bevacizumab, cetuximab or panitumumab) used during induction, scheduled to initiate prior to the first day of treatment with the study drug
Last CT scan at 3 weeks or less before the first day of treatment

### Subject Withdrawal and Discontinuation.

During the initial study period (12W), if a patient experiences disease progression and needs to start a second-line therapy, the patient will be withdrawn from the study.
During the second part of the study (after 2nd dose) if a patient experiences disease progression and needs to start a second-line therapy, the patient will remain in the study, receive the third vaccination as scheduled and complete follow-up.
Transient local erythema and edema at the site of vaccination were observed as expected, as well as a flu-like syndrome with minor fever and fatigue. These reactions are already well-known for peptide vaccination and usually are associated with the mechanism of action, because fever and flu-like syndrome might be the consequence and sign for the induction of immune responses (this is known as typical vaccine reactions for childhood vaccinations).
Only one serious adverse event (SAE) “possibly related” to the vaccine was recorded (Table 21).
One dose limiting toxicity (DLT) not related to the vaccine occurred (syncope).

Safety results are summarized in Table 21.

TABLE 21
Serious adverse events reported in the OBERTO clinical trial.
No related SAE occurred (only 1 “possibly related”).

| Patient ID | SAE | Relatedness |
|---|---|---|
| 010001 | Death due to disease progression | Unrelated |
| 010004 | Embolism | Unlikely Related |
| 010004 | Abdominal pain | Unrelated |
| 010007 | Bowel Obstruction | Unrelated |
| 020004 | Non-Infectious Acute Encephalitis | Possibly Related |

### Example 13—Expression Frequency Based Target Antigen Selection During Vaccine Design and It's Clinical Validation for mCRC

Shared tumor antigens enable precise targeting of all tumor types—including the ones with low mutational burden. Population expression data collected previously from 2,391 CRC biopsies represents the variability of antigen expression in CRC patients worldwide (FIG. 12A).

PolyPEPI1018 is a peptide vaccine we designed to contain 12 unique epitopes derived from 7 conserved testis specific antigens (TSAs) frequently expressed in mCRC. In our model we supposed, that by selecting the TSA frequently expressed in CRC, the target identification will be correct and will eliminate the need for tumor biopsy. We have calculated that the probability of 3 out of 7 TSAs being expressed in each tumor is greater than 95%. (FIG. 12B)

In a phase I study we evaluated the safety, tolerability and immunogenicity of PolyPEPI1018 as an add-on to maintenance therapy in subjects with metastatic colorectal cancer (mCRC) (NCT03391232) (See also in Example 4).

Immunogenicity measurements proved pre-existing immune responses and indirectly confirmed target antigen expression in the patients. Immunogenicty was measured with enriched Fluorospot assay (ELISPOT) from PBMC samples isolated prior to vaccination and in different time points following a following single immunization with PolyPEPI1018 to confirm vaccine-induced T cell responses; PBMC samples were in vitro stimulated with vaccine-specific peptides (9 mers and 30 mers) to determine vaccine-induced T cell responses above baseline. In average 4, at least 2 patients had pre-existing CD8 T cell responses against each target antigen (FIG. 12C). 7 out of 10 patients had pre-existing immune responses against at least 1 antigen (average 3) (FIG. 12D). These results provide proof for the proper target selection, because CD8+ T cell response for a CRC specific target TSA prior to vaccination with PolyPEPI1018 vaccine confirms the expression of that target antigen in the analyzed patient. Targeting the real (expressed) TSAs is the prerequisite for an effective tumor vaccine.

### Example 14—Pre-Clinical and Clinical Immunogenicity of PolyPEPI1018 Vaccine Proves Proper Peptide Selection

PolyPEPI1018 vaccine contains six 30 mer peptides, each designed by joining two immunogenic 15 mer fragments (each involving a 9 mer PEPI, consequently there are 2 PEPIs in each 30 mer by design) derived from 7 TSAs (FIG. 13). These antigens are frequently expressed in CRC tumors based on analysis of 2,391 biopsies (FIG. 12).

Preclinical immunogenicity results calculated for the Model Population (n=433) and for a CRC cohort (n=37) resulted in 98% and 100% predicted immunogenicity based on PEPI test predictions and this was clinically proved in the OBERTO trial (n=10), with immune responses measured for at least one antigen in 90% of patients. More interestingly, 90% of patients had vaccine peptide specific immune responses against at least 2 antigens and 80% had CD8+ T cell response against 3 or more different vaccine antigens, showing evidence for appropriate target antigen selection during the design of PolyPEPI1018. CD4+ T cell specific and CD8+ T cell specific clinical immunogenicity is detailed in Table 22. High immune response rates were found for both effector and memory effector T cells, both for CD4+ and CD8+ T cells, and 9 of 10 patients' immune responses were boosted or de novo induced by the vaccine. Also, the fractions of CRC-reactive, polyfunctional CD8+ and CD4+ T cells have been increased in patient's PBMC after vaccination by 2.5- and 13-fold, respectively.

TABLE 22
Clinical immunogenicity results for PolyPEPI1018 in mCRC.

| Immunological responses | % Patients (n) | |
|---|---|---|
| CD4+ T cell responses | 100% | (10/10) |
| CD8+ T cell responses against ≥3 antigens | 80% | (8/10) |
| Both CD8+ and CD4+ T cell responses | 90% | (9/10) |
| Ex vivo detected CD8+ T cell response | 71% | (5/7) |
| Ex vivo detected CD4+ T cell response | 86% | (6/7) |

| | |
|---|---|
| Average increase of the fraction of | 0.39% |
| polyfunctional (IFN-γ and TNF-α positive) | |
| CD8+ T cells compared to pre-vaccination | |
| Average increase of the fraction of | 0.066% |
| polyfunctional (IL-2 and TNF-α positive) | |
| CD4+ T cells compared to pre-vaccination | |

### Example 15—Clinical Response for PolyPEPI1018 Treatment

The OBERTO clinical trial (NCT03391232), that has been further described in Examples 4, 12, 13 and 14 was analyzed for preliminary objective tumor response rates (RECIST 1.1) (FIGS. 14A-C). Of the eleven vaccinated patients on maintenance therapy, 5 had stable disease (SD) at the time point of the preliminary analysis (12 weeks), 3 experienced unexpected tumor responses (partial response, PR) observed on treatment (maintenance therapy+ vaccination) and 3 had progressed disease (PD) according to RECIST 1.1 criteria. Stable disease as best response was achieved in 69% of patients on maintenance therapy (capecitabine and bevacizumab). Patient 020004 had durable treatment effect after 12 weeks, and patient 010004 had long lasting treatment effect, qualified for curative surgery. Following the 3^{rd} vaccination this patient had no evidence of disease thus being complete responder, as shown on the swimmer plot on FIGS. 14A-C.

After one vaccination, ORR was 27%, DCR was 63%, and in patients receiving at least 2 doses (out of the 3 doses), 2 of 5 had ORR (40%) and DCR was as high as 80% (SD+PR+CR in 4 out of 5 patients) (Table 23).

TABLE 23
Clinical response for PolyPEPI1018 treatment after ≥1 and ≥2 vaccination dose

| | Objective Response Rate | | | |
|---|---|---|---|---|
| Number of vaccination dose | (CR + PR) | | Disease Control Rate (SD + PR + CR) | |
| ≥1 | 27% | (3/11) | 63% | (7/11) |
| ≥2 | 40% | (2/5) | 80% | (4/5) |

Based on the data of the 5 patients receiving multiple doses of PolyPEPI1018 vaccine in the OBERTO-101 clinical trial, preliminary data suggests that higher AGP count (>2) is associated with longer PFS and elevated tumor size reduction (FIGS. 14B and C).

### Example 16—Selection of Peptides for Treatment of Cancer

Based on the discovery of the role of PEPIs in T cell activation as described herein, a method was developed for designing peptides for the treatment of cancer. Specifically the peptides were designed to stimulate T cell responses against known tumor associated antigens in the maximum number of human subjects.

192 TSAs were selected that are known to be expressed in one or more of 19 cancer indications (Table 24). Data concerning expression rates of the TSA in the different cancer indications, where available in peer reviewed publications, was used to rank the TSA in each indication by expression frequency. The ranking order for the TSA is different in each indication.

TABLE 24
TSA by indication and Expression Rate (ER)

| | | | |
|---|---|---|---|
| # | AG ID NO | Antigen | % |

BREAST

| | | | |
|---|---|---|---|
| 1 | 111 | PIWIL1 | 100% |
| 2 | 112 | PIWIL2 | 94% |
| 3 | 131 | SPAG9 | 88% |
| 4 | 8 | AKAP-4 | 85% |
| 5 | 36 | EpCAM | 76% |
| 6 | 62 | JARID1B | 76% |
| 7 | 27 | CTCFL | 74% |
| 8 | 154 | TSGA10 | 70% |
| 9 | 140 | Survivin | 68% |
| 10 | 19 | CDCA1 | 64% |
| 11 | 102 | ODF4 | 63% |
| 12 | 110 | PEPP2 | 60% |
| 13 | 71 | MAGE-A11 | 59% |
| 14 | 160 | XAGE-1 | 58% |
| 15 | 42 | FMR1NB | 55% |
| 16 | 168 | ATAD2 | 52% |
| 17 | 191 | WBP2NL | 50% |
| 18 | 31 | DBPC | 50% |
| 19 | 116 | PRAME | 49% |
| 20 | 59 | HOM-TES-85 | 47% |
| 21 | 96 | NY-BR-1 | 47% |
| 22 | 129 | SP17 | 47% |
| 23 | 79 | MAGE-A9 | 44% |
| 24 | 43 | FSIP1 | 42% |
| 25 | 2 | ACRBP | 40% |
| 26 | 7 | AKAP-3 | 40% |
| 27 | 145 | TDRD1 | 38% |
| 28 | 41 | FBXO39 | 38% |
| 29 | 123 | SCP-1 | 37% |
| 30 | 66 | LDHC | 35% |
| 31 | 67 | LEMD1 | 35% |
| 32 | 70 | MAGE-A10 | 34% |
| 33 | 69 | MAGE-A1 | 31% |
| 34 | 49 | GAGE-7 | 31% |
| 35 | 130 | SPAG1 | 31% |
| 36 | 22 | CT46 | 29% |
| 37 | 185 | SPAG8 | 29% |
| 38 | 74 | MAGE-A3 | 28% |
| 39 | 138 | SSX-4 | 27% |
| 40 | 132 | SPAN-Xc | 26% |
| 41 | 184 | SPAG6 | 21% |
| 42 | 33 | DKKL11 | 20% |
| 43 | 86 | MAGE-C2 | 19% |
| 44 | 85 | MAGE-C1 | 16% |
| 45 | 20 | CRISP2 | 15% |
| 46 | 98 | NY-ESO-1 | 15% |
| 47 | 77 | MAGE-A6 | 15% |
| 48 | 21 | CT45 | 15% |
| 49 | 55 | HAGE | 14% |
| 50 | 44 | FTHL17 | 14% |
| 51 | 30 | CXorf61 | 13% |
| 52 | 65 | Lage-1 | 12% |
| 53 | 72 | MAGE-A12 | 12% |
| 54 | 153 | TRAG-3 | 10% |
| 55 | 10 | BAGE-1 | 9% |
| 56 | 135 | SSX-1 | 9% |
| 57 | 75 | MAGE-A4 | 9% |
| 58 | 76 | MAGE-A5 | 9% |
| 59 | 63 | KOC1 | 8% |
| 60 | 136 | SSX-2 | 7% |
| 61 | 159 | WT1 | 7% |
| 62 | 127 | SLCO6A1 | 7% |
| 63 | 115 | PLAC1 | 4% |
| 64 | 23 | CT47 | 4% |
| 65 | 121 | SAGE1 | 2% |
| 66 | 95 | NXF2 | 2% |
| 67 | 149 | TEX15 | 0% |
| 68 | 147 | TEX101 | 0% |
| 69 | 142 | TAF7L | 0% |
| 70 | 139 | SSX-5 | 0% |
| 71 | 137 | SSX-3 | 0% |
| 72 | 134 | SPO11 | 0% |
| 73 | 101 | ODF3 | 0% |
| 74 | 88 | MORC1 | 0% |
| 75 | 17 | CALR3 | 0% |
| 76 | 4 | ADAM2 | 0% |

OVARIAN

| | | | |
|---|---|---|---|
| 1 | 36 | EpCAM | 92% |
| 2 | 131 | SPAG9 | 90% |
| 3 | 8 | AKAP-4 | 89% |
| 4 | 113 | PIWIL3 | 88% |
| 5 | 140 | Survivin | 85% |
| 6 | 153 | TRAG-3 | 83% |
| 7 | 27 | CTCFL | 82% |
| 8 | 62 | JARID1B | 71% |
| 9 | 111 | PIWIL1 | 68% |
| 10 | 2 | ACRBP | 65% |
| 11 | 179 | KIF20A | 65% |
| 12 | 159 | WT1 | 63% |
| 13 | 129 | SP17 | 62% |
| 14 | 112 | PIWIL2 | 61% |
| 15 | 116 | PRAME | 59% |
| 16 | 33 | DKKL1 | 57% |
| 17 | 7 | AKAP-3 | 48% |
| 18 | 66 | LDHC | 43% |
| 19 | 63 | KOC1 | 43% |
| 20 | 70 | MAGE-A10 | 38% |
| 21 | 79 | MAGE-A9 | 37% |
| 22 | 75 | MAGE-A4 | 37% |
| 23 | 154 | TSGA10 | 35% |
| 24 | 143 | TAG-1 | 35% |
| 25 | 21 | CT45 | 33% |
| 26 | 59 | HOM-TES-85 | 32% |
| 27 | 130 | SPAG1 | 31% |
| 28 | 35 | DPPA2 | 31% |
| 29 | 74 | MAGE-A3 | 30% |
| 30 | 124 | SCP3a | 25% |
| 31 | 69 | MAGE-A1 | 24% |
| 32 | 98 | NY-ESO-1 | 23% |
| 33 | 65 | Lage-1 | 22% |
| 34 | 115 | PLAC1 | 21% |
| 35 | 31 | DBPC | 20% |
| 36 | 85 | MAGE-C1 | 20% |
| 37 | 161 | XAGE-1b | 18% |
| 38 | 123 | SCP-1 | 15% |
| 39 | 88 | MORC1 | 14% |
| 40 | 20 | CRISP2 | 14% |
| 41 | 144 | TAG-2a | 13% |
| 42 | 138 | SSX-4 | 13% |
| 43 | 162 | XAGE-1c | 11% |
| 44 | 136 | SSX-2 | 7% |
| 45 | 73 | MAGE-A2 | 7% |
| 46 | 163 | XAGE-1d | 6% |
| 47 | 160 | XAGE-1 | 5% |
| 48 | 135 | SSX-1 | 2% |
| 49 | 139 | SSX-5 | 0% |
| 50 | 137 | SSX-3 | 0% |
| 51 | 134 | SPO11 | 0% |
| 52 | 132 | SPAN-Xc | 0% |
| 53 | 18 | CCDC62 | 0% |
| 54 | 4 | ADAM2 | 0% |

PANCREATIC

| | | | |
|---|---|---|---|
| 1 | 171 | CCDC110 | 100% |
| 2 | 181 | MSLN | 96% |
| 3 | 130 | SPAG1 | 93% |
| 4 | 36 | EpCAM | 85% |
| 5 | 174 | DKK1 | 84% |
| 6 | 140 | Survivin | 83% |
| 7 | 159 | WT1 | 72% |
| 8 | 111 | PIWIL1 | 71% |
| 9 | 179 | KIF20A | 63% |
| 10 | 1 | 5T4 | 62% |
| 11 | 31 | DBPC | 60% |
| 12 | 172 | CEA | 60% |
| 13 | 63 | KOC1 | 54% |
| 14 | 115 | PLAC1 | 44% |
| 15 | 64 | KU-CT-1 | 33% |
| 16 | 123 | SCP-1 | 31% |
| 17 | 27 | CTCFL | 29% |
| 18 | 69 | MAGE-A1 | 27% |
| 19 | 170 | CASC5 | 27% |
| 20 | 178 | hTERT | 21% |
| 21 | 138 | SSX-4 | 21% |
| 22 | 46 | GAGE-2 | 17% |
| 23 | 74 | MAGE-A3 | 11% |
| 24 | 65 | Lage-1 | 7% |
| 25 | 136 | SSX-2 | 4% |
| 26 | 98 | NY-ESO-1 | 4% |
| 27 | 75 | MAGE-A4 | 2% |
| 28 | 70 | MAGE-A10 | 1% |
| 29 | 137 | SSX-3 | 0% |
| 30 | 135 | SSX-1 | 0% |
| 31 | 132 | SPAN-Xc | 0% |
| 32 | 85 | MAGE-C1 | 0% |

BRAIN

| | | | |
|---|---|---|---|
| 1 | 166 | ZNF165 | 100% |
| 2 | 109 | PBK | 100% |
| 3 | 178 | hTERT | 100% |
| 4 | 175 | EZH2 | 100% |
| 5 | 37 | EPHA2 | 95% |
| 6 | 61 | IL13RA2 | 95% |
| 7 | 157 | TYR | 91% |
| 8 | 140 | Survivin | 87% |
| 9 | 54 | gp100 | 85% |
| 10 | 71 | MAGE-A11 | 85% |
| 11 | 154 | TSGA10 | 83% |
| 12 | 44 | FTHL17 | 82% |
| 13 | 159 | WT1 | 81% |
| 14 | 111 | PIWIL1 | 76% |
| 15 | 112 | PIWIL2 | 64% |
| 16 | 165 | XAGE-3 | 60% |
| 17 | 131 | SPAG9 | 60% |
| 18 | 128 | SOX-6 | 60% |
| 19 | 179 | KIF20A | 58% |
| 20 | 116 | PRAME | 56% |
| 21 | 3 | ACTL8 | 54% |
| 22 | 1 | 5T4 | 50% |
| 23 | 103 | OIP5 | 48% |
| 24 | 186 | SPINLW1 | 47% |
| 25 | 74 | MAGE-A3 | 42% |
| 26 | 45 | GAGE-1 | 42% |
| 27 | 69 | MAGE-A1 | 41% |
| 28 | 63 | KOC1 | 38% |
| 29 | 55 | HAGE | 38% |
| 30 | 29 | CXorf48 | 35% |
| 31 | 86 | MAGE-C2 | 34% |
| 32 | 108 | PASD1 | 31% |
| 33 | 73 | MAGE-A2 | 28% |
| 34 | 127 | SLC06A1 | 19% |
| 35 | 160 | XAGE-1 | 18% |
| 36 | 95 | NXF2 | 18% |
| 37 | 36 | EpCAM | 16% |
| 38 | 123 | SCP-1 | 14% |
| 39 | 129 | SP17 | 13% |
| 40 | 85 | MAGE-C1 | 13% |
| 41 | 134 | SPO11 | 12% |
| 42 | 75 | MAGE-A4 | 11% |
| 43 | 59 | HOM-TES-85 | 10% |
| 44 | 27 | CTCFL | 9% |
| 45 | 39 | FAM46D | 8% |
| 46 | 84 | MAGE-B6 | 6% |
| 47 | 136 | SSX-2 | 4% |
| 48 | 138 | SSX-4 | 4% |
| 49 | 98 | NY-ESO-1 | 4% |
| 50 | 65 | Lage-1 | 2% |
| 51 | 153 | TRAG-3 | 0% |
| 52 | 152 | TPTE | 0% |
| 53 | 187 | SSX-7 | 0% |
| 54 | 139 | SSX-5 | 0% |
| 55 | 137 | SSX-3 | 0% |
| 56 | 135 | SSX-1 | 0% |
| 57 | 121 | SAGE1 | 0% |
| 58 | 87 | MART | 0% |
| 59 | 83 | MAGE-B4 | 0% |
| 60 | 81 | MAGE-B2 | 0% |
| 61 | 80 | MAGE-B1 | 0% |
| 62 | 30 | CXorf61 | 0% |
| 63 | 21 | CT45 | 0% |
| 64 | 14 | BRDT | 0% |
| 65 | 80 | MAGE-B1 | 0% |
| 66 | 30 | CXorf61 | 0% |
| 67 | 21 | CT45 | 0% |
| 68 | 14 | BRDT | 0% |

HCC

| | | | |
|---|---|---|---|
| 1 | 19 | CDCA1 | 100% |
| 2 | 129 | SP17 | 87% |
| 3 | 159 | WT1 | 86% |
| 4 | 2 | ACRBP | 70% |
| 5 | 135 | SSX-1 | 69% |
| 6 | 63 | KOC1 | 66% |
| 7 | 109 | PBK | 64% |
| 8 | 81 | MAGE-B2 | 61% |
| 9 | 132 | SPAN-Xc | 60% |
| 10 | 146 | TEKT5 | 60% |
| 11 | 60 | IGFS11 | 60% |
| 12 | 69 | MAGE-A1 | 56% |
| 13 | 27 | CTCFL | 55% |
| 14 | 140 | Survivin | 54% |
| 15 | 166 | ZNF165 | 52% |
| 16 | 131 | SPAG9 | 52% |
| 17 | 171 | CCDC110 | 50% |
| 18 | 7 | AKAP-3 | 50% |
| 19 | 40 | FATE1 | 49% |
| 20 | 138 | SSX-4 | 48% |
| 21 | 80 | MAGE-B1 | 47% |
| 22 | 136 | SSX-2 | 46% |
| 23 | 78 | MAGE-A8 | 45% |
| 24 | 36 | EpCAM | 41% |
| 25 | 115 | PLAC1 | 41% |
| 26 | 111 | PIWIL1 | 40% |
| 27 | 31 | DBPC | 40% |
| 28 | 16 | CAGE1 | 39% |
| 29 | 152 | TPTE | 39% |
| 30 | 86 | MAGE-C2 | 37% |
| 31 | 139 | SSX-5 | 37% |
| 32 | 65 | Lage-1 | 36% |
| 33 | 77 | MAGE-A6 | 36% |
| 34 | 72 | MAGE-A12 | 36% |
| 35 | 71 | MAGE-A1 | 36% |
| 36 | 156 | TSPY1 | 35% |
| 37 | 74 | MAGE-A3 | 34% |
| 38 | 160 | XAGE-1 | 33% |
| 39 | 62 | JARID1B | 29% |
| 40 | 153 | TRAG-3 | 26% |
| 41 | 123 | SCP-1 | 25% |
| 42 | 73 | MAGE-A2 | 25% |
| 43 | 29 | CXorf48 | 25% |
| 44 | 154 | TSGA10 | 20% |
| 45 | 85 | MAGE-C1 | 20% |
| 46 | 59 | HOM-TES-85 | 19% |
| 47 | 98 | NY-ESO-1 | 17% |
| 48 | 70 | MAGE-A10 | 15% |
| 49 | 188 | TDRD4 | 14% |
| 50 | 130 | SPAG1 | 11% |
| 51 | 75 | MAGE-A4 | 7% |
| 52 | 79 | MAGE-A9 | 5% |
| 53 | 18 | CCDC62 | 0% |

THYROID

| | | | |
|---|---|---|---|
| 1 | 159 | WT1 | 97% |
| 2 | 112 | PIWIL2 | 88% |
| 3 | 36 | EpCAM | 83% |
| 4 | 131 | SPAG9 | 78% |
| 5 | 63 | KOC1 | 65% |
| 6 | 116 | PRAME | 60% |
| 7 | 140 | Survivin | 53% |
| 8 | 108 | PASD1 | 25% |
| 9 | 66 | LDHC | 25% |
| 10 | 85 | MAGE-C1 | 22% |
| 11 | 74 | MAGE-A3 | 22% |
| 12 | 95 | NXF2 | 20% |
| 13 | 55 | HAGE | 20% |
| 14 | 69 | MAGE-A1 | 17% |
| 15 | 51 | GASZ | 13% |
| 16 | 129 | SP17 | 10% |
| 17 | 73 | MAGE-A2 | 9% |
| 18 | 39 | FAM46D | 8% |
| 19 | 136 | SSX-2 | 8% |
| 20 | 98 | NY-ESO-1 | 7% |
| 21 | 65 | Lage-1 | 4% |
| 22 | 70 | MAGE-A10 | 2% |
| 23 | 157 | TYR | 0% |
| 24 | 149 | TEX15 | 0% |
| 25 | 145 | TDRD1 | 0% |
| 26 | 142 | TAF7L | 0% |
| 27 | 139 | SSX-5 | 0% |
| 28 | 138 | SSX-4 | 0% |
| 29 | 137 | SSX-3 | 0% |
| 30 | 135 | SSX-1 | 0% |
| 31 | 134 | SPO11 | 0% |
| 32 | 123 | SCP-1 | 0% |
| 33 | 121 | SAGE1 | 0% |
| 34 | 119 | RAGE-1 | 0% |
| 35 | 92 | NKX3.1 | 0% |
| 36 | 88 | MORC1 | 0% |
| 37 | 87 | MART1 | 0% |
| 38 | 86 | MAGE-C2 | 0% |
| 39 | 75 | MAGE-A4 | 0% |
| 40 | 44 | FTHL17 | 0% |
| 41 | 33 | DKKL1 | 0% |
| 42 | 20 | CRISP2 | 0% |
| 43 | 1 | 5T4 | 0% |
| 44 | 1 | 5T4 | 0% |

LUNG

| | | | |
|---|---|---|---|
| 1 | 179 | KIF20A | 95% |
| 2 | 173 | CEP55 | 94% |
| 3 | 32 | DCAF12 | 89% |
| 4 | 19 | CDCA1 | 85% |
| 5 | 36 | EpCAM | 78% |
| 6 | 130 | SPAG1 | 71% |
| 7 | 192 | ZNF645 | 68% |
| 8 | 115 | PLAC1 | 68% |
| 9 | 112 | PIWIL2 | 66% |
| 10 | 116 | PRAME | 63% |
| 11 | 63 | KOC1 | 62% |
| 12 | 140 | Survivin | 59% |
| 13 | 131 | SPAG9 | 56% |
| 14 | 62 | JARID1B | 55% |
| 15 | 153 | TRAG-3 | 54% |
| 16 | 77 | MAGE-A6 | 51% |
| 17 | 39 | FAM46D | 50% |
| 18 | 171 | CCDC110 | 50% |
| 19 | 22 | CT46 | 47% |
| 20 | 7 | AKAP-3 | 46% |
| 21 | 81 | MAGE-B2 | 43% |
| 22 | 161 | XAGE-1b | 43% |
| 23 | 185 | SPAG8 | 43% |
| 24 | 69 | MAGE-A1 | 42% |
| 25 | 170 | CASC5 | 41% |
| 26 | 73 | MAGE-A2 | 40% |
| 27 | 177 | HSPB9 | 40% |
| 28 | 93 | NLRP4 | 38% |
| 29 | 64 | KU-CT-1 | 38% |
| 30 | 14 | BRDT | 37% |
| 31 | 74 | MAGE-A3 | 37% |
| 32 | 152 | TPTE | 36% |
| 33 | 27 | CTCFL | 36% |
| 34 | 30 | CXorf61 | 34% |
| 35 | 55 | HAGE | 32% |
| 36 | 21 | CT45 | 32% |
| 37 | 150 | MEG | 31% |
| 38 | 158 | VCX | 31% |
| 39 | 72 | MAGE-A12 | 31% |
| 40 | 31 | DBPC | 30% |
| 41 | 35 | DPPA2 | 30% |
| 42 | 20 | CRISP2 | 29% |
| 43 | 184 | SPAG6 | 29% |
| 44 | 108 | PASD1 | 29% |
| 45 | 66 | LDHC | 29% |
| 46 | 51 | GASZ | 29% |
| 47 | 65 | Lage-1 | 28% |
| 48 | 42 | FMR1NB | 28% |
| 49 | 75 | MAGE-A4 | 27% |
| 50 | 86 | MAGE-C2 | 27% |
| 51 | 160 | XAGE-1 | 25% |
| 52 | 44 | FTHL17 | 25% |
| 53 | 8 | AKAP-4 | 25% |
| 54 | 70 | MAGE-A10 | 24% |
| 55 | 167 | COX6B2 | 24% |
| 56 | 168 | ATAD2 | 23% |
| 57 | 166 | ZNF165 | 21% |
| 58 | 149 | TEX15 | 21% |
| 59 | 182 | PTTG-1 | 20% |
| 60 | 2 | ACRBP | 20% |
| 61 | 85 | MAGE-C1 | 19% |
| 62 | 88 | MORC1 | 18% |
| 63 | 98 | NY-ESO-1 | 17% |
| 64 | 132 | SPAN-Xc | 17% |
| 65 | 169 | CABYR | 16% |
| 66 | 111 | PIWIL1 | 15% |
| 67 | 95 | NXF2 | 15% |
| 68 | 29 | CXorf48 | 15% |
| 69 | 121 | SAGE1 | 15% |
| 70 | 23 | CT47 | 14% |
| 71 | 16 | CAGE1 | 14% |
| 72 | 80 | MAGE-B1 | 13% |
| 73 | 138 | SSX-4 | 13% |
| 74 | 163 | XAGE-ld | 13% |
| 75 | 61 | IL13RA2 | 12% |
| 76 | 33 | DKKL11 | 12% |
| 77 | 129 | SP17 | 12% |
| 78 | 141 | SYCE1 | 11% |
| 79 | 59 | HOM-TES-85 | 11% |
| 80 | 10 | BAGE-1 | 10% |
| 81 | 136 | SSX-2 | 10% |
| 82 | 146 | TEKT5 | 10% |
| 83 | 142 | TAF7L | 9% |
| 84 | 17 | CALR3 | 9% |
| 85 | 135 | SSX-1 | 8% |
| 86 | 113 | PIWIL3 | 7% |
| 87 | 5 | ADAM29 | 7% |
| 88 | 127 | SLCO6A1 | 7% |
| 89 | 145 | TDRD1 | 5% |
| 90 | 176 | HORMAD2 | 5% |
| 91 | 139 | SSX-5 | 4% |
| 92 | 123 | SCP-1 | 1% |
| 93 | 4 | ADAM2 | 1% |
| 94 | 162 | XAGE-1c | 0% |
| 95 | 156 | TSPY1 | 0% |
| 96 | 134 | SPO11 | 0% |
| 97 | 83 | MAGE-B4 | 0% |
| 98 | 82 | MAGE-B3 | 0% |
| 99 | 68 | LIPI | 0% |
| 100 | 41 | FBXO39 | 0% |
| 101 | 40 | FATE1 | 0% |

BLADDER

| | | | |
|---|---|---|---|
| 1 | 19 | CDCA1 | 100% |
| 2 | 89 | MPHOSPH1 | 90% |
| 3 | 112 | PIWIL2 | 82% |
| 4 | 131 | SPAG9 | 81% |
| 5 | 140 | Survivin | 62% |
| 6 | 79 | MAGE-A9 | 61% |
| 7 | 78 | MAGE-A8 | 57% |
| 8 | 36 | EpCAM | 54% |
| 9 | 24 | CTAGE1 | 53% |
| 10 | 25 | CTAGE2 | 49% |
| 11 | 109 | PBK | 43% |
| 12 | 74 | MAGE-A3 | 42% |
| 13 | 154 | TSGA10 | 38% |
| 14 | 69 | MAGE-A1 | 34% |
| 15 | 98 | NY-ESO-1 | 31% |
| 16 | 65 | Lage-1 | 30% |
| 17 | 72 | MAGE-A12 | 29% |
| 18 | 145 | TDRD1 | 29% |
| 19 | 2 | ACRBP | 28% |
| 20 | 70 | MAGE-A10 | 28% |
| 21 | 85 | MAGE-C1 | 27% |
| 22 | 75 | MAGE-A4 | 27% |
| 23 | 77 | MAGE-A6 | 25% |
| 24 | 73 | MAGE-A2 | 25% |
| 25 | 55 | HAGE | 24% |
| 26 | 44 | FTHL17 | 22% |
| 27 | 149 | TEX15 | 21% |
| 28 | 63 | KOC1 | 20% |
| 29 | 22 | CT46 | 20% |
| 30 | 116 | PRAME | 19% |
| 31 | 86 | MAGE-C2 | 19% |
| 32 | 95 | NXF2 | 19% |
| 33 | 136 | SSX-2 | 13% |
| 34 | 121 | SAGE1 | 12% |
| 35 | 138 | SSX-4 | 11% |
| 36 | 142 | TAF7L | 10% |
| 37 | 132 | SPAN-Xc | 9% |
| 38 | 135 | SSX-1 | 7% |
| 39 | 123 | SCP-1 | 2% |
| 40 | 156 | TSPY1 | 0% |
| 41 | 81 | MAGE-B2 | 0% |
| 42 | 80 | MAGE-B1 | 0% |
| 43 | 59 | HOM-TES-85 | 0% |

LEUKEMIAS

| | | | |
|---|---|---|---|
| 1 | 131 | SPAG9 | 100% |
| 2 | 38 | FAM133A | 83% |
| 3 | 26 | cTAGE5 | 74% |
| 4 | 24 | CTAGE1 | 72% |
| 5 | 85 | MAGE-C1 | 70% |
| 6 | 74 | MAGE-A3 | 65% |
| 7 | 98 | NY-ESO-1 | 65% |
| 8 | 62 | JARID1B | 60% |
| 9 | 166 | ZNF165 | 52% |
| 10 | 65 | Lage-1 | 49% |
| 11 | 124 | SCP3a | 47% |
| 12 | 28 | CTNNA2 | 45% |
| 13 | 86 | MAGE-C2 | 37% |
| 14 | 75 | MAGE-A4 | 36% |
| 15 | 135 | SSX-1 | 31% |
| 16 | 138 | SSX-4 | 30% |
| 17 | 139 | SSX-5 | 24% |
| 18 | 116 | PRAME | 23% |
| 19 | 72 | MAGE-A12 | 21% |
| 20 | 69 | MAGE-A1 | 20% |
| 21 | 136 | SSX-2 | 12% |
| 22 | 156 | TSPY1 | 12% |
| 23 | 123 | SCP-1 | 11% |
| 24 | 73 | MAGE-A2 | 11% |
| 25 | 79 | MAGE-A9 | 9% |
| 26 | 68 | LIPI | 6% |
| 27 | 4 | ADAM2 | 6% |
| 28 | 148 | TEX14 | 5% |
| 29 | 80 | MAGE-B1 | 5% |
| 30 | 105 | PAGE2 | 4% |
| 31 | 132 | SPAN-Xc | 4% |
| 32 | 137 | SSX-3 | 4% |
| 33 | 83 | MAGE-B4 | 3% |
| 34 | 70 | MAGE-A10 | 0% |

MELANOMA

| | | | |
|---|---|---|---|
| 1 | 62 | JARID1B | 100% |
| 2 | 140 | Survivin | 96% |
| 3 | 116 | PRAME | 90% |
| 4 | 87 | MART1 | 89% |
| 5 | 157 | TYR | 87% |
| 6 | 54 | gp100 | 82% |
| 7 | 132 | SPAN-Xc | 70% |
| 8 | 77 | MAGE-A6 | 65% |
| 9 | 179 | KIF20A | 64% |
| 10 | 151 | TMEM31 | 63% |
| 11 | 73 | MAGE-A2 | 62% |
| 12 | 74 | MAGE-A3 | 60% |
| 13 | 119 | RAGE-1 | 60% |
| 14 | 171 | CCDC110 | 60% |
| 15 | 71 | MAGE-A11 | 54% |
| 16 | 161 | XAGE-1b | 50% |
| 17 | 72 | MAGE-A12 | 49% |
| 18 | 63 | KOC1 | 46% |
| 19 | 85 | MAGE-C1 | 45% |
| 20 | 70 | MAGE-A10 | 45% |
| 21 | 86 | MAGE-C2 | 45% |
| 22 | 108 | PASD1 | 44% |
| 23 | 17 | CALR3 | 44% |
| 24 | 66 | LDHC | 44% |
| 25 | 163 | XAGE-1d | 43% |
| 26 | 98 | NY-ESO-1 | 37% |
| 27 | 69 | MAGE-A1 | 37% |
| 28 | 136 | SSX-2 | 35% |
| 29 | 65 | Lage-1 | 35% |
| 30 | 75 | MAGE-A4 | 30% |
| 31 | 162 | XAGE-1c | 29% |
| 32 | 39 | FAM46D | 28% |
| 33 | 149 | TEX15 | 27% |
| 34 | 27 | CTCFL | 27% |
| 35 | 10 | BAGE-1 | 26% |
| 36 | 154 | TSGA10 | 25% |
| 37 | 135 | SSX-1 | 25% |
| 38 | 76 | MAGE-A5 | 25% |
| 39 | 33 | DKKL1 | 25% |
| 40 | 11 | BAGE-2 | 23% |
| 41 | 107 | PAGES | 22% |
| 42 | 81 | MAGE-B2 | 22% |
| 43 | 142 | TAF7L | 21% |
| 44 | 138 | SSX-4 | 21% |
| 45 | 80 | MAGE-B1 | 19% |
| 46 | 88 | MORC1 | 19% |
| 47 | 55 | HAGE | 17% |
| 48 | 51 | GASZ | 17% |
| 49 | 123 | SCP-1 | 16% |
| 50 | 164 | XAGE-2 | 14% |
| 51 | 12 | BAGE-3 | 14% |
| 52 | 164 | XAGE-2 | 14% |
| 53 | 12 | BAGE-3 | 14% |
| 54 | 79 | MAGE-A9 | 12% |
| 55 | 78 | MAGE-A8 | 10% |
| 56 | 13 | BAGE-5 | 9% |
| 57 | 95 | NX2 | 8% |
| 58 | 160 | XAGE-1 | 7% |
| 59 | 134 | SPO11 | 6% |
| 60 | 20 | CRISP2 | 6% |
| 61 | 139 | SSX-5 | 5% |
| 62 | 121 | SAGE1 | 5% |
| 63 | 4 | ADAM2 | 3% |
| 64 | 165 | XAGE-3 | 0% |
| 65 | 145 | TDRD1 | 0% |
| 66 | 68 | LIPI | 0% |
| 67 | 64 | KU-CT-1 | 0% |
| 68 | 44 | FTHL17 | 0% |
| 69 | 168 | ATAD2 | 0% |

GASTRIC

| | | | |
|---|---|---|---|
| 1 | 140 | Survivin | 100% |
| 2 | 35 | DPPA2 | 100% |
| 3 | 171 | CCDC110 | 100% |
| 4 | 36 | EpCAM | 90% |
| 5 | 7 | AKAP-3 | 89% |
| 6 | 60 | IGFS11 | 88% |
| 7 | 30 | CXorf61 | 80% |
| 8 | 16 | CAGE1 | 77% |
| 9 | 111 | PIWIL1 | 76% |
| 10 | 155 | TSP50 | 57% |
| 11 | 1 | 5T4 | 52% |
| 12 | 63 | KOC1 | 52% |
| 13 | 31 | DBPC | 50% |
| 14 | 103 | OIP5 | 48% |
| 15 | 108 | PASD1 | 44% |
| 16 | 168 | ATAD2 | 43% |
| 17 | 146 | TEKT5 | 40% |
| 18 | 17 | CALR3 | 40% |
| 19 | 74 | MAGE-A3 | 37% |
| 20 | 73 | MAGE-A2 | 31% |
| 21 | 69 | MAGE-A1 | 31% |
| 22 | 70 | MAGE-A10 | 30% |
| 23 | 75 | MAGE-A4 | 24% |
| 24 | 138 | SSX-4 | 23% |
| 25 | 29 | CXorf48 | 21% |
| 26 | H6 | PRAME | 21% |
| 27 | 123 | SCP-1 | 14% |
| 28 | 65 | Lage-1 | 14% |
| 29 | 135 | SSX-1 | 13% |
| 30 | 98 | NY-ESO-1 | 12% |
| 31 | 39 | FAM46D | 11% |
| 32 | 153 | TRAG-3 | 10% |
| 33 | 86 | MAGE-C2 | 10% |
| 34 | 166 | ZNF165 | 9% |
| 35 | 161 | XAGE-1b | 9% |
| 36 | 121 | SAGE1 | 8% |
| 37 | 21 | CT45 | 8% |
| 38 | 18 | CCDC62 | 7% |
| 39 | 95 | NXF2 | 6% |
| 40 | 85 | MAGE-C1 | 6% |
| 41 | 41 | FBXO39 | 4% |
| 42 | 136 | SSX-2 | 3% |
| 43 | 40 | FATE1 | 2% |
| 44 | 156 | TSPY1 | 0% |
| 45 | 152 | TPTE | 0% |
| 46 | 139 | SSX-5 | 0% |
| 47 | 132 | SPAN-Xc | 0% |
| 48 | 81 | MAGE-B2 | 0% |
| 49 | 80 | MAGE-B1 | 0% |
| 50 | 51 | GASZ | 0% |
| 51 | 19 | CDCA1 | 0% |
| 52 | 2 | ACRBP | 0% |

Cervical

| | | | |
|---|---|---|---|
| 1 | 112 | PIWIL2 | 100% |
| 2 | 1 | 5T4 | 100% |
| 3 | 16 | CAGE1 | 98% |
| 4 | 140 | Survivin | 92% |
| 5 | 8 | AKAP-4 | 86% |
| 6 | 66 | LDHC | 83% |
| 7 | 111 | PIWIL1 | 83% |
| 8 | 131 | SPAG9 | 82% |
| 9 | 63 | KOC1 | 68% |
| 10 | 129 | SP17 | 61% |
| 11 | 108 | PASD1 | 58% |
| 12 | 51 | GASZ | 50% |
| 13 | 98 | NY-ESO-1 | 42% |
| 14 | 39 | FAM46D | 37% |
| 15 | 36 | EpCAM | 36% |
| 16 | 179 | KIF20A | 35% |
| 17 | 134 | SPO11 | 33% |
| 18 | 33 | DKKL1 | 33% |
| 19 | 27 | CTCFL | 32% |
| 20 | 65 | Lage-1 | 26% |
| 21 | 75 | MAGE-A4 | 23% |
| 22 | 138 | SSX-4 | 20% |
| 23 | 19 | CDCA1 | 18% |
| 24 | 69 | MAGE-A1 | 17% |
| 26 | 20 | CRISP2 | 17% |
| 26 | 74 | MAGE-A3 | 16% |
| 27 | 159 | WT1 | 5% |
| 28 | 136 | SSX-2 | 4% |
| 29 | 135 | SSX-1 | 4% |
| 30 | 88 | MORC1 | 0% |
| 31 | 70 | MAGE-A10 | 0% |
| 32 | 4 | ADAM2 | 0% |

PROSTATE

| | | | |
|---|---|---|---|
| 1 | 115 | PLAC1 | 100% |
| 2 | 112 | PIWIL2 | 100% |
| 3 | 106 | PAGE4 | 100% |
| 4 | 62 | JARID1B | 100% |
| 5 | 36 | EpCAM | 98% |
| 6 | 27 | CTCFL | 98% |
| 7 | 133 | SPATA19 | 88% |
| 8 | 19 | CDCA1 | 85% |
| 9 | 140 | Survivin | 83% |
| 10 | 92 | NKX3.1 | 70% |
| 11 | 8 | AKAP-4 | 65% |
| 12 | 32 | DCAF12 | 59% |
| 13 | 145 | TDRD1 | 47% |
| 14 | 31 | DBPC | 40% |
| 15 | 66 | LDHC | 38% |
| 16 | 20 | CRISP2 | 38% |
| 17 | 90 | MUC-1 | 34% |
| 18 | 39 | FAM46D | 33% |
| 19 | 108 | PASD1 | 29% |
| 20 | 136 | SSX-2 | 28% |
| 21 | 130 | SPAG1 | 27% |
| 22 | 94 | NR6A1 | 25% |
| 23 | 4 | ADAM2 | 25% |
| 24 | 34 | DMRT1 | 24% |
| 25 | 67 | LEMD1 | 23% |
| 26 | 77 | MAGE-A6 | 23% |
| 27 | 55 | HAGE | 22% |
| 28 | 18 | CCDC62 | 22% |
| 29 | 98 | NY-ESO-1 | 21% |
| 30 | 73 | MAGE-A2 | 18% |
| 31 | 154 | TSGA10 | 15% |
| 32 | 74 | MAGE-A3 | 15% |
| 33 | 95 | NXF2 | 14% |
| 34 | 69 | MAGE-A1 | 13% |
| 35 | 149 | TEX15 | 13% |
| 36 | 33 | DKKL1 | 13% |
| 37 | 146 | TEKT5 | 11% |
| 38 | 129 | SP17 | 10% |
| 39 | 116 | PRAME | 10% |
| 40 | 100 | ODF2 | 10% |
| 41 | 99 | ODF1 | 10% |
| 42 | 70 | MAGE-A10 | 10% |
| 43 | 64 | KU-CT-1 | 10% |
| 44 | 65 | Lage-1 | 8% |
| 45 | 135 | SSX-1 | 7% |
| 46 | 86 | MAGE-C2 | 6% |
| 47 | 72 | MAGE-A12 | 5% |
| 48 | 111 | PIWIL1 | 4% |
| 49 | 2 | ACRBP | 4% |
| 50 | 117 | PRSS55 | 1% |
| 51 | 142 | TAF7L | 0% |
| 52 | 134 | SPO11 | 0% |
| 53 | 102 | ODF4 | 0% |
| 54 | 101 | ODF3 | 0% |
| 55 | 88 | MORC1 | 0% |
| 56 | 81 | MAGE-B2 | 0% |
| 57 | 80 | MAGE-B1 | 0% |
| 58 | 75 | MAGE-A4 | 0% |
| 59 | 63 | KOC1 | 0% |
| 60 | 44 | FTHL17 | 0% |
| 61 | 42 | FMR1NB | 0% |
| 62 | 10 | BAGE-1 | 0% |
| 63 | 168 | ATAD2 | 0% |

CRC

| | | | |
|---|---|---|---|
| 1 | 180 | MCAK | 100% |
| 2 | 113 | PIWIL3 | 96% |
| 3 | 67 | LEMD1 | 94% |
| 4 | 159 | WT1 | 91% |
| 5 | 31 | DBPC | 90% |
| 6 | 155 | TSP50 | 89% |
| 7 | 36 | EpCAM | 88% |
| 8 | 140 | Survivin | 87% |
| 9 | 1 | 5T4 | 85% |
| 10 | 7 | AKAP-3 | 83% |
| 11 | 27 | CTCFL | 80% |
| 12 | 112 | PIWIL2 | 80% |
| 13 | 16 | CAGE1 | 74% |
| 14 | 8 | AKAP-4 | 74% |
| 15 | 131 | SPAG9 | 71% |
| 16 | 63 | KOC1 | 65% |
| 17 | 2 | ACRBP | 63% |
| 18 | 168 | ATAD2 | 58% |
| 19 | 130 | SPAG1 | 55% |
| 20 | 60 | IGFS11 | 55% |
| 21 | 146 | TEKT5 | 50% |
| 22 | 171 | CCDC110 | 50% |
| 23 | 111 | PIWIL1 | 48% |
| 24 | 108 | PASD1 | 44% |
| 25 | 78 | MAGE-A8 | 44% |
| 26 | 35 | DPPA2 | 44% |
| 27 | 166 | ZNF165 | 43% |
| 28 | 177 | HSPB9 | 40% |
| 29 | 19 | CDCA1 | 40% |
| 30 | 41 | FBXO39 | 39% |
| 31 | 106 | PAGE4 | 33% |
| 32 | 26 | cTAGE5 | 31% |
| 33 | 97 | NYD-TSPG | 30% |
| 34 | 115 | PLAC1 | 27% |
| 35 | 132 | SPAN-Xc | 24% |
| 36 | 51 | GASZ | 22% |
| 37 | 39 | FAM46D | 22% |
| 38 | 77 | MAGE-A6 | 22% |
| 39 | 74 | MAGE-A3 | 22% |
| 40 | 40 | FATE1 | 21% |
| 41 | 24 | CTAGE1 | 19% |
| 42 | 75 | MAGE-A4 | 18% |
| 43 | 73 | MAGE-A2 | 18% |
| 44 | 29 | CXorf48 | 17% |
| 45 | 17 | CALR3 | 16% |
| 46 | 66 | LDHC | 15% |
| 47 | 55 | HAGE | 15% |
| 48 | 69 | MAGE-A1 | 14% |
| 49 | 65 | Lage-1 | 13% |
| 50 | 18 | CCDC62 | 13% |
| 51 | 107 | PAGES | 11% |
| 52 | 21 | CT45 | 10% |
| 53 | 95 | NXF2 | 9% |
| 54 | 119 | RAGE-1 | 8% |
| 55 | 76 | MAGE-A5 | 7% |
| 56 | 22 | CT46 | 7% |
| 57 | 153 | TRAG-3 | 7% |
| 58 | 72 | MAGE-A12 | 6% |
| 59 | 123 | SCP-1 | 6% |
| 60 | 136 | SSX-2 | 5% |
| 61 | 154 | TSGA10 | 5% |
| 62 | 86 | MAGE-C2 | 4% |
| 63 | 152 | TPTE | 4% |
| 64 | 116 | PRAME | 4% |
| 65 | 98 | NY-ESO-1 | 3% |
| 66 | 70 | MAGE-A10 | 3% |
| 67 | 135 | SSX-1 | 2% |
| 68 | 139 | SSX-5 | 2% |
| 69 | 85 | MAGE-C1 | 1% |
| 70 | 121 | SAGE1 | 1% |
| 71 | 149 | TEX15 | 0% |
| 72 | 145 | TDRD1 | 0% |
| 73 | 142 | TAF7L | 0% |
| 74 | 137 | SSX-3 | 0% |
| 75 | 134 | SPO11 | 0% |
| 76 | 104 | PAGE1 | 0% |
| 77 | 88 | MORC1 | 0% |
| 78 | 81 | MAGE-B2 | 0% |
| 79 | 80 | MAGE-B1 | 0% |
| 80 | 68 | LIPI | 0% |
| 81 | 44 | FTHL17 | 0% |
| 82 | 33 | DKKL1 | 0% |
| 83 | 20 | CRISP2 | 0% |
| 84 | 10 | BAGE-1 | 0% |
| 85 | 4 | ADAM2 | 0% |
| 86 | 10 | BAGE-1 | 0% |
| 87 | 4 | ADAM2 | 0% |

LYMPHOMAS

| | | | |
|---|---|---|---|
| 1 | 123 | SCP-1 | 39% |
| 2 | 74 | MAGE-A3 | 17% |
| 3 | 138 | SSX-4 | 16% |
| 4 | 136 | SSX-2 | 16% |
| 5 | 59 | HOM-TES-85 | 14% |
| 6 | 85 | MAGE-C1 | 13% |
| 7 | 98 | NY-ESO-1 | 11% |
| 8 | 69 | MAGE-A1 | 9% |
| 9 | 135 | SSX-1 | 6% |
| 10 | 156 | TSPY1 | 0% |
| 11 | 86 | MAGE-C2 | 0% |

PEDIATRIC

| | | | |
|---|---|---|---|
| 1 | 37 | EPHA2 | 100% |
| 2 | 77 | MAGE-A6 | 80% |
| 3 | 135 | SSX-1 | 76% |
| 4 | 74 | MAGE-A3 | 76% |
| 5 | 116 | PRAME | 75% |
| 6 | 122 | SART3 | 73% |
| 7 | 138 | SSX-4 | 67% |
| 8 | 73 | MAGE-A2 | 60% |
| 9 | 61 | IL13RA2 | 58% |
| 10 | 160 | XAGE-1 | 53% |
| 11 | 136 | SSX-2 | 44% |
| 12 | 69 | MAGE-A1 | 44% |
| 13 | 98 | NY-ESO-1 | 42% |
| 14 | 75 | MAGE-A4 | 41% |
| 15 | 72 | MAGE-A12 | 41% |
| 16 | 86 | MAGE-C2 | 40% |
| 17 | 85 | MAGE-C1 | 39% |
| 18 | 70 | MAGE-A10 | 33% |
| 19 | 164 | XAGE-2 | 14% |
| 20 | 65 | Lage-1 | 7% |

Kidney

| | | | |
|---|---|---|---|
| 1 | 19 | CDCA1 | 100% |
| 2 | 131 | SPAG9 | 88% |
| 3 | 17 | CALR3 | 80% |
| 4 | 66 | LDHC | 57% |
| 5 | 36 | EpCAM | 55% |
| 6 | 79 | MAGE-A9 | 55% |
| 7 | 111 | PIWIL1 | 52% |
| 8 | 112 | PIWIL2 | 49% |
| 9 | 107 | PAGES | 44% |
| 10 | 116 | PRAME | 40% |
| 11 | 140 | Survivin | 40% |
| 12 | 119 | RAGE-1 | 37% |
| 13 | 149 | TEX15 | 33% |
| 14 | 74 | MAGE-A3 | 27% |
| 15 | 68 | LIPI | 25% |
| 16 | 18 | CCDC62 | 25% |
| 17 | 63 | KOC1 | 22% |
| 18 | 75 | MAGE-A4 | 17% |
| 19 | 20 | CRISP2 | 14% |
| 20 | 64 | KU-CT-1 | 13% |
| 21 | 130 | SPAG1 | 11% |
| 22 | 69 | MAGE-A1 | 11% |
| 23 | 31 | DBPC | 10% |
| 24 | 4 | ADAM2 | 9% |
| 25 | 123 | SCP-1 | 8% |
| 26 | 73 | MAGE-A2 | 7% |
| 27 | 55 | HAGE | 6% |
| 28 | 156 | TSPY1 | 5% |
| 29 | 121 | SAGE1 | 5% |
| 30 | 72 | MAGE-A12 | 4% |
| 31 | 98 | NY-ESO-1 | 2% |
| 32 | 136 | SSX-2 | 2% |
| 33 | 65 | Lage-1 | 2% |
| 34 | 70 | MAGE-A10 | 1% |
| 35 | 145 | TDRD1 | 0% |
| 36 | 142 | TAF7L | 0% |
| 37 | 139 | SSX-5 | 0% |
| 38 | 138 | SSX-4 | 0% |
| 39 | 137 | SSX-3 | 0% |
| 40 | 135 | SSX-1 | 0% |
| 41 | 134 | SPO11 | 0% |
| 42 | 95 | NXF2 | 0% |
| 43 | 88 | MORC1 | 0% |
| 44 | 85 | MAGE-C1 | 0% |
| 45 | 81 | MAGE-B2 | 0% |
| 46 | 80 | MAGE-B1 | 0% |
| 47 | 77 | MAGE-A6 | 0% |
| 48 | 44 | FTHL17 | 0% |
| 49 | 42 | FMR1NB | 0% |
| 50 | 33 | DKKL1 | 0% |
| 51 | 14 | BRDT | 0% |
| 52 | 10 | BAGE-1 | 0% |
| 53 | 2 | ACRBP | 0% |

HEAD and NECK

| | | | |
|---|---|---|---|
| 1 | 140 | Survivin | 91% |
| 2 | 171 | CCDC110 | 67% |
| 3 | 116 | PRAME | 61% |
| 4 | 77 | MAGE-A6 | 58% |
| 5 | 81 | MAGE-B2 | 45% |
| 6 | 79 | MAGE-A9 | 44% |
| 7 | 75 | MAGE-A4 | 42% |
| 8 | 160 | XAGE-1 | 40% |
| 9 | 135 | SSX-1 | 40% |
| 10 | 74 | MAGE-A3 | 40% |
| 11 | 45 | GAGE-1 | 39% |
| 12 | 73 | MAGE-A2 | 38% |
| 13 | 71 | MAGE-A11 | 37% |
| 14 | 72 | MAGE-A12 | 34% |
| 15 | 63 | KOC1 | 29% |
| 16 | 138 | SSX-4 | 29% |
| 17 | 86 | MAGE-C2 | 29% |
| 18 | 29 | CXorf48 | 27% |
| 19 | 69 | MAGE-A1 | 23% |
| 20 | 145 | TDRD1 | 22% |
| 21 | 55 | HAGE | 20% |
| 22 | 98 | NY-ESO-1 | 20% |
| 23 | 121 | SAGE1 | 17% |
| 24 | 136 | SSX-2 | 17% |
| 25 | 85 | MAGE-C1 | 15% |
| 26 | 70 | MAGE-A10 | 12% |
| 27 | 165 | XAGE-3 | 12% |
| 28 | 123 | SCP-1 | 12% |
| 29 | 103 | IP5 | 12% |
| 30 | 18 | CCDC62 | 12% |
| 31 | 65 | Lage-1 | 11% |
| 32 | 20 | CRISP2 | 11% |
| 33 | 149 | TEX15 | 11% |
| 34 | 142 | TAF7L | 10% |
| 35 | 44 | FTHL17 | 10% |
| 36 | 49 | GAGE-7 | 9% |
| 37 | 139 | SSX-5 | 7% |
| 38 | 130 | SPAG1 | 7% |
| 39 | 10 | BAGE-1 | 6% |
| 40 | 164 | XAGE-2 | 6% |
| 41 | 107 | PAGES | 6% |
| 42 | 95 | NXF2 | 5% |
| 43 | 59 | HOM-TES-85 | 4% |
| 44 | 96 | NY-BR-1 | 2% |
| 45 | 152 | TPTE | 0% |
| 46 | 146 | TEKT5 | 0% |
| 47 | 134 | SPO11 | 0% |
| 48 | 68 | LIPI | 0% |
| 49 | 66 | LDHC | 0% |

Esophageal

| | | | |
|---|---|---|---|
| 1 | 1 | 5T4 | 100% |
| 2 | 189 | TTK | 97% |
| 3 | 159 | WT1 | 88% |
| 4 | 36 | EpCAM | 81% |
| 5 | 129 | SP17 | 81% |
| 6 | 75 | MAGE-A4 | 76% |
| 7 | 63 | KOC1 | 74% |
| 8 | 111 | PIWIL1 | 70% |
| 9 | 77 | MAGE-A6 | 68% |
| 10 | 140 | Survivin | 67% |
| 11 | 74 | MAGE-A3 | 66% |
| 12 | 27 | CTCFL | 56% |
| 13 | 79 | MAGE-A9 | 55% |
| 14 | 153 | TRAG-3 | 55% |
| 15 | 73 | MAGE-A2 | 53% |
| 16 | 69 | MAGE-A1 | 46% |
| 17 | 22 | CT46 | 35% |
| 18 | 127 | SLCO6A1 | 33% |
| 19 | 65 | Lage-1 | 31% |
| 20 | 98 | NY-ESO-1 | 31% |
| 21 | 21 | CT45 | 28% |
| 22 | 70 | MAGE-A10 | 27% |
| 23 | 55 | HAGE | 27% |
| 24 | 72 | MAGE-A12 | 26% |
| 25 | 121 | SAGE1 | 22% |
| 26 | 19 | CDCA1 | 22% |
| 27 | 18 | CCDC62 | 21% |
| 28 | 149 | TEX15 | 20% |
| 29 | 177 | HSPB9 | 20% |
| 30 | 95 | NXF2 | 17% |
| 31 | 169 | CABYR | 17% |
| 32 | 179 | KIF20A | 15% |
| 33 | 23 | CT47 | 15% |
| 34 | 85 | MAGE-C1 | 12% |
| 35 | 86 | MAGE-C2 | 11% |
| 36 | 145 | TDRD1 | 10% |
| 37 | 42 | FMR1NB | 8% |
| 38 | 138 | SSX-4 | 7% |
| 39 | 10 | BAGE-1 | 6% |
| 40 | 123 | SCP-1 | 2% |
| 41 | 142 | TAF7L | 0% |
| 42 | 136 | SSX-2 | 0% |
| 43 | 135 | SSX-1 | 0% |
| 44 | 132 | SPAN-Xc | 0% |
| 45 | 44 | FTHL17 | 0% |

A model population was used that comprises 15,693 subjects with up to 500 male and 500 female subjects from each of a broad range of ethnicities. The full 6 HLA class I and DQ & DRB1 class II alleles is available for each subject. The number of HLA class II bindings was duplicated to simulate the full genome.

For each of the 15,693 subjects all 15 mer amino acid sequences in each TSA were identified that met the following HLA-binding criteria: (i) predicted to bind to at least four HLA class II alleles of the subject (HLA class II-binding PEPI4+); and (ii) comprise a 9 mer amino acid sequence that is predicted to bind to at least three HLA class I of the subject (HLA class I-binding PEPI3+).

A hotspot was identified in the amino acid sequence of each TSA, wherein the hotspot is a 20 mer that comprises a 15 mer that meets the HLA binding criteria for the maximum number of subjects in the 15,693 subject population. The hotspot analysis is illustrated in FIG. 15.

The hotspot analysis was repeated in a further 29 cycles, or until no more sequences meeting the HLA-binding criteria could be identified. Hotspot sequences were screened against manufacturing feasibility criteria. Any hotspot sequence that contained a cysteine residue, or that had a calculated hydrophilicity of less than 33%, was rejected and a different hotspot sequence comprising a 15 mer that met the HLA binding criteria for the next highest number of subjects was selected instead.

In each cycle subjects for whom the HLA-binding criteria were met for any hotspot sequence selected in any previous cycle were excluded. In this way the hotspots that were selected maximized, for each cycle, the number of subjects in the population for whom a hotspot sequence had been selected that is predicted to induce both CD4+ and CD8+ T cell responses. The hotspot sequences selected in each cycle and the TSA of which they are a fragment are shown in Table 25. A total of 3286 hotspot sequences were selected. FIG. 16 shows the distribution of hotspot sequence selection across the 192 CTA.

TABLE 25A
Hotspot Sequences and corresponding TAA

| | SEQ | | Source Antigen(s) | | SEQ | | Source Antigen(s) |
|---|---|---|---|---|---|---|---|
| | ID | | [AA position | | ID | | [AA position |
| Cycle | NO | Sequence | in Antigen] | Cycle | NO | Sequence | in Antigen] |
| 1 | 1 | PTSSASSFSSSAPFLASAVS | 5T4 [34,53] | 11 | 1394 | AESMYGDFQEAMTHLQHKLI | FAM46D |
| | | | | | | | [204,223] |
| 1 | 2 | VESTPMIMENIQELIRSAQE | ACRBP [275,294] | 11 | 1395 | VAQTSLEEFNVLEMEVMRRQ | FATE1 |
| | | | | | | | [121,140] |
| 1 | 3 | LNWEGVQYLWSFVLENHRRE | ACTL8 [73,92] | 11 | 1396 | KYRKLIESELSYFVIVYSVM | FBXO39 |
| | | | | | | | [423,442] |
| 1 | 4 | PHNERVYSYSGTGIMKPLDQ | ADAM2 [67,86] | 11 | 1397 | STEPGSFKVDTASNLNSGKE | FSIP1 [35,54] |
| 1 | 5 | RIVEIVVVIDNYLYIRYERN | ADAM29 [198,217] | 11 | 1398 | LENFERYFLRLSDDKMEHAQ | FTHL17 [49,68] |
| 1 | 6 | RETFMNKFIYEIARRHPFLY | AFP [155,174] | 11 | 1399 | PVNSQKITLEWASPQNFTSV | GAS2 [382,401] |
| 1 | 7 | DEVSFYANRLTNLVIAMARK | AKAP-3 [122,141] | 11 | 1400 | NRPLIKPKRRLSAARRAGTS | GATA-3 |
| | | | | | | | [298,317] |
| 1 | 8 | IDDLSFYVNRLSSLVIQMAH | AKAP-4 [214,233] | 11 | 1401 | EAFEIVVRHAKNYTNAMFKN | Glypican-3 |
| | | | | | | | [113,132] |
| 1 | 9 | RTKKELASALKSALSGHLET | ANXA2 [78,97] | 11 | 1402 | QLHDPSGYLAEADLSYTWDF | gp100 |
| | | | | | | | [243,262] |
| 1 | 10 | ARAVFLALSAQLLQARLMKE | BAGE-1 [3,22] | 11 | 1403 | IPEELVSMAERFKAHQQKRE | HAGE [613,632] |
| 1 | 11 | GVVFLALSAQLLQARLMKEE | BAGE-2 [4,23]; | 11 | 1404 | KQQTDIAVNWAGGLHHAKKS | HDAC1 |
| | | | BAGE-3 [4,23] | | | | [126,145] |
| 1 | 12 | GAVFLALSAQLLQARLMKEE | BAGE-5 [4,23] | 11 | 1405 | HNLLLNYGLYRKMEIYRPHK | HDAC2 [40,59] |
| 1 | 13 | NQEYKDAYKFAADVRLMFMN | BRDT [328,347] | 11 | 1406 | RFHSEDYIDFLQRVSPTNMQ | HDAC3 [60,79] |
| 1 | 14 | PSNINQFAAAYFQELTMYRG | CABYP [30,49] | 11 | 1407 | TLVVTANRGSSPQVMSRSNG | IGFS11 |
| | | | | | | | [371,390] |
| 1 | 15 | KRASQLASKMHSLLALMVGL | CAGE1 [608,627] | 11 | 1408 | IRSSYFTFQLQNIVKPLPPV | IL13RA2 |
| | | | | | | | [223,242] |
| 1 | 16 | QTTQNGRFYAISARFKPFSN | CALR3 [67,86] | 11 | 1409 | RDYTLRTFGEMADAFKSDYF | JARID1B |
| | | | | | | | [375,394] |
| 1 | 17 | SPASELIAIQDSHSLGSSKS | CCDC62 [566,585] | 11 | 1410 | EEIPLKILAHNNFVGRLIGK | KOC1 [275,294] |
| 1 | 18 | AKRTSRFLSGIINFIHFREA | CDCA1 [114,133] | 11 | 1411 | GGLKKLLSFAENSTIPDIQK | KU-CT-1 |
| | | | | | | | [274,293] |
| 1 | 19 | EGKDPAFTALLTTQLQVQRE | CRISP2 [20,39] | 11 | 1412 | DLQHGSLFFSTSKITSGKDY | LDHC [64,83] |
| 1 | 20 | QGPTAVRKRFFESIIKEAAR | CT45 [152,171] | 11 | 1413 | VKNTRKVAVSLSVHIKNLLK | LIPI [147,166] |
| 1 | 21 | ATAQLQRTPMSALVFETKIS | CT46 [2,21] | 11 | 1414 | KVLEYVIKVSARVRFFFPSL | MAGE-A1 |
| | | | | | | | [278,297] |
| 1 | 22 | LDMVHSLLHRLSHNDHILIE | CT47 [124,143] | 11 | 1415 | TSLGLTYDGMLSDVQSMPKT | MAGE-A10 |
| | | | | | | | [205,224] |
| 1 | 23 | GNNFIQNFYLPQNYIDQFLL | CTAGE1 [22,41] | 11 | 1416 | SFSQDILHDKIIDLVHLLLR | MAGE-A11 |
| | | | | | | | [216,235] |
| 1 | 24 | FAVLFLWRSERSVISRLYVR | CTAGE2 [24,43] | 11 | 1417 | APEEKIWEELSVLEASEGRE | MAGE-A12 |
| | | | | | | | [216,235] |
| 1 | 25 | FAVLEFLWRSERSVRSRLYV | CTAGE5 [53,72] | 11 | 1418 | ELVHFLLLKYRAREPVTKAE | MAGE-A2 |
| | | | | | | | [115,134]; |
| | | | | | | | MAGE-A3 |
| | | | | | | | [115,134] |
| 1 | 26 | HTRFTQSGTMKIHILQKHGE | CTCFL [404,423] | 11 | 1419 | ESAGPPQSPQGASALPTTIS | MAGE-A4 |
| | | | | | | | [58,77] |
| 1 | 27 | KKNATMLYTASQAFLRHETV | CTNNA2 [214,233] | 11 | 1420 | GSVVGNWQYFFPVIFSKASD | MAGE-A6 |
| | | | | | | | [137,156] |
| 1 | 28 | NEDNIYISNSIYESIAIVSE | CXorf48 [122,141] | 11 | 1421 | TKAEMLESVIKNYKNHEITI | MAGE-A8 |
| | | | | | | | [134,153] |
| 1 | 29 | NTGEMSSNSTALALVRPSSS | CXorf61 [27,46] | 11 | 1422 | EPRKLLTQDWVQENYLEYRQ | MAGE-A9 |
| | | | | | | | [240,259] |
| 1 | 30 | PPPFFYRRRFVRGPRPPNQQ | DBPC [225,244] | 11 | 1423 | MLMHFILRKYKMREFIMKAD | MAGE-B1 |
| | | | | | | | [114,133] |
| 1 | 31 | SRVLHGYAAQQLPSLLKERE | DCAF12 [64,83] | 11 | 1424 | FGLELNKVNPNGHTYTFIDK | MAGE-B2 |
| | | | | | | | [164,183] |
| 1 | 32 | NLLRGIDSLFSAPMDFRGLP | DKK11 [63,82] | 11 | 1425 | SKMKVLEFWAKVNKTVPSAF | MAGE-B3 |
| | | | | | | | [284,303] |
| 1 | 33 | IAERQRVMAAQVALRRQQAQ | DMRT1 [108,127] | 11 | 1426 | FRKVSQRTELVFGLALKEVN | MAGE-B4 |
| | | | | | | | [151,170] |
| 1 | 34 | GKKIEVYLRLHRHAYPEQRQ | DPPA2 [113,132] | 11 | 1427 | SASQKAIIFKRLSKDAVKKK | MAGE-B6 |
| | | | | | | | [179,198] |
| 1 | 35 | SERVRTYWIIIELKHKAREK | EpCAM [136,155] | 11 | 1428 | LQIPVSPSSSSILLSLFQSF | MAGE-C1 |
| | | | | | | | [233,252] |
| 1 | 36 | KGWELMQNIMNDMPIYMYSV | EPHA2 [50,69] | 11 | 1429 | KDYFPVILKRAREFMELLFG | MAGE-C2 |
| | | | | | | | [175,194] |
| 1 | 37 | KRDNRVAYMNPIAMARWRGP | FAM133A [3,22] | 11 | 1430 | NEVLAMKRSSSLPSWKSLLN | MORC1 |
| | | | | | | | [759,778] |
| 1 | 38 | RRQFEFSVDSFQIVLDPMLD | FAM46D [166,185] | 11 | 1431 | AFTKLNNASSRSHSIFTVKI | MPHOSPH1 |
| | | | | | | | [340,359] |
| 1 | 39 | GENQEHLVIAEMMELGSRSR | FATE1 [26,45] | 11 | 1432 | RRKNYGQLDIFPARDTYHPM | MUC-1 |
| | | | | | | | [1187,1206] |
| 1 | 40 | EHLEVKFMNPYNAVLTKKFQ | FBXO39 [90,109] | 11 | 1433 | RAHLAKNLKLTETQVKIWFQ | NKX3.1 |
| | | | | | | | [154,173] |
| 1 | 41 | SGSSYFVLANGHILPNSENA | FMR1NB [92,111] | 11 | 1434 | EKLDAFFGFQLSQEIKQQIH | NLRP4 |
| | | | | | | | [513,532] |
| 1 | 42 | ISDTKDYFMSKTLGIGRLKR | FSIP1 [515,534] | 11 | 1435 | QSYSLIHQLLSAEDLEPLGT | NR6A1 |
| | | | | | | | [249,268] |
| 1 | 43 | GWESGLVAMESAFHLEKNVN | FTH117 [93,112] | 11 | 1436 | YLKGELLRRTKRDIVDSLSA | NXF2 [454,473] |
| 1 | 44 | EEMRSHYVAQTGILWLLMNN | GAGE-1 [109,128] | 11 | 1437 | SLVEKTPDEAASLVEGTSDK | NY-BR-1 |
| | | | | | | | [263,282] |
| 1 | 45 | SRGKSTYYWPRPRRYVQPPE | GAGE-3 [4,23] | 11 | 1438 | KPAELSRGRGILIFSDFKDF | NYD-TSPG |
| | | | | | | | [212,231] |
| 1 | 46 | WRGRSTYYWPRPRRYVQPPE | GAGE-6 [3,22]; | 11 | 1439 | EAARRQFQSQLADLQQLPDI | ODF2 [583,602] |
| | | | GAGE-7 [3,22] | | | | |
| 1 | 47 | EKFKKAMTIGDVSLVQELLD | OASZ [48,67] | 11 | 1440 | LTPGPGDYFPEKSTKYVFDS | ODF3 [99,118] |
| 1 | 48 | ALSRHMSSLSHISPFSHSSH | GATA-3 [396,415] | 11 | 1441 | PFQWRITHSFRWMAQVLASE | ODF4 [67,86] |
| 1 | 49 | EFVGEFFTDVSLYILGSDIN | Glypican-3 | 11 | 1442 | SVHLAWDLSRSLGAVVFSRV | OIP5 [90,109] |
| | | | [143,162] | | | | |
| 1 | 50 | DGGNKHFLRNQPLTFALQLH | gp100 [226,245] | 11 | 1443 | EHGESSAYENVKFIVNVRDI | PASD1 |
| | | | | | | | [118,137] |
| 1 | 51 | RLNDLQMSNFVNLKNITYLV | HAGE [375,394] | 11 | 1444 | GTGVNVYLMKRSPRGLSHSP | PBK [41,60] |
| 1 | 52 | KPHRIRMTHNLLLNYGLYRK | HDAC1 [31,50]; | 11 | 1445 | TEADRVIQRTNSMQQLEQWI | PEPP2 |
| | | | HDAC2 [32,51] | | | | [399,418] |
| 1 | 53 | TIFENLKMLNHAPSVQIHDV | HDAC3 [361,380] | 11 | 1446 | KRVNTRFFAQSGGRLQNPLP | PIWIL1 |
| | | | | | | | [742,761] |
| 1 | 54 | KKVNFLDMSLDDIIIYKELE | HOM-TES-85 [21,40] | 11 | 1447 | AGPIGMRMSPPAWVELKDDR | PIWIL2 |
| | | | | | | | [629,648] |
| 1 | 55 | LINLNVIWMVTPLSNANQPE | IGFS11 [52,71] | 11 | 1448 | HDIVNRQKSIAGFVASTNAE | PIWIL3 |
| | | | | | | | [657,676] |
| 1 | 56 | PIRSSYFTFQLQNIVKPLPP | IL13RA2 [222,241] | 11 | 1449 | LNKWYKYNHDLPARIIVYRA | PIWIL4 |
| | | | | | | | [674,693] |
| 1 | 57 | KLRYRYTLDDLYPMMNALKL | JARID1B [731,750] | 11 | 1450 | KKLKIFAMPMQDIKMILKMV | PRAME |
| | | | | | | | [211,230] |
| 1 | 58 | PDESWALKAIEALSGKIELH | KOC1 [45,64] | 11 | 1451 | YSEELFPEELSVVLGINDLT | PRSS55 |
| | | | | | | | [111,130] |
| 1 | 59 | IQNHDIMHAIISPLRSANTV | KU-CT-1 [435,454] | 11 | 1452 | EENSRLLQERGVAYINADSS | PSMA [436,455] |
| 1 | 60 | PDSRLLQLHITMPFSSPMEA | Lage-1 [83,102] | 11 | 1453 | YELIRGRRYPLSEKKIMHYM | RAGE-1 |
| | | | | | | | [90,109] |
| 1 | 61 | QRNVAIMKSIIPAIVHYSPD | LDHC [111,130] | 11 | 1454 | RPRRINMTDTGISPMSTRDP | SAGE1 |
| | | | | | | | [226,245] |
| 1 | 62 | SEELNIILQGNIILSTEKSK | LEMD1 [67,86] | 11 | 1455 | AIVEAARLEKVHSLFRRQLA | SART3 |
| | | | | | | | [237,256] |
| 1 | 63 | QKYSAHAFQFSPRNVLWLLV | LIPI [12,31] | 11 | 1456 | DSDPALQKVNFLPVLEQVGN | SCP-1 [63,82] |
| 1 | 64 | VKVLEYVIKVSARVRFFFPS | MAGE-A1 [277,296] | 11 | 1457 | DVIEGKTAVIEKRRKKRSSA | SCP3a [41,60] |
| 1 | 65 | DETTAMASASSSATGSFSYP | MAGE-A10 | 11 | 1458 | VAFPPRAKDGLVVFGKNSAR | SCRN1 [11,30] |
| | | | [349,368] | | | | |
| 1 | 66 | DPTSHSYVLVTSLNLSYDGI | MAGE-A11 | 11 | 1459 | EAQNKELKTQVALSSETPRT | se57-1 |
| | | | [283,302] | | | | [289,308] |
| 1 | 67 | ALSRKMAELVHFLLLKYRAR | MAGE-A12 | 11 | 1460 | SSGISFQSKYLSFFILGQTV | SLCO6A1 |
| | | | [108,127] | | | | [219,238] |
| 1 | 68 | GREDSVFAHPRKLLMQDLVQ | MAGE-A2 [233,252] | 11 | 1461 | MITQLISLREQLLAAHDEQK | SOX-6 [194,213] |
| 1 | 69 | FPVIFSKASSSLQLVFGIEL | MAGE-A3 [147,166] | 11 | 1462 | DLNKVILLDPSIIEAKMELE | SPAG1 |
| | | | | | | | [713,732] |
| 1 | 70 | VNARVRIAYPSLREAALLEE | MAGE-A4 [294,313] | 11 | 1463 | GGSGAVMSERVSGLAGSIYR | SPAG9 [14,33] |
| 1 | 71 | KSPQGASAIPTAIDFTLWRQ | MAGE-A5 [63,82] | 11 | 1464 | FLPITSSDIDVVESEAVSVL | SPATA19 |
| | | | | | | | [20,39] |
| 1 | 72 | KKLLTQYFVQENYLEYRQVP | MAGE-A6 [243,262] | 11 | 1465 | ILSTSKGLIAGNLRYIEEDG | SPO11 |
| | | | | | | | [170,189] |
| 1 | 73 | RAPEEAIWEALSVMGLYDGR | MAGE-A8 [218,237] | 11 | 1466 | YMKRKYEAMTKLGFKAILPS | SSX-3 [50,69] |
| 1 | 74 | PAQLEFMFQEALKLKVAELV | MAGE-A9 [97,116] | 11 | 1467 | QKVGSLEPRVEVLINRINEV | SYCE1 [44,63] |
| 1 | 75 | DNPSGHTYTLVSKLNLTNEG | MAGE-B1 [168,187] | 11 | 1468 | TVRNLARSQSVKMKDKLKID | TAF7L [113,132] |
| 1 | 76 | SSDPPRFQFLWGPRAYAETS | MAGE-B2 [265,284] | 11 | 1469 | EVHIVRNGGTSMMVENMAVR | TAG-1 |
| | | | | | | | [997,1016] |
| 1 | 77 | LIMKTNMLVQFLMEMYKMKK | MAGE-B3 [111,130] | 11 | 1470 | KLVENSLSISNPGLFTSLGP | TDRD1 |
| | | | | | | | [144,163] |
| 1 | 78 | SSSVLRETASSSLAFGIPQE | MAGE-B4 [42,61] | 11 | 1471 | TLKLRLRETQDTLQLLVMTK | TEKT5 [428,447] |
| 1 | 79 | PQILNRTSQHLVVAFGVELK | MAGE-B6 [234,253] | 11 | 1472 | SREFTNAYKLPLAVGPPSLN | TEX14 |
| | | | | | | | [781,800] |
| 1 | 80 | RPVSSFFSYTLASLLQSSHE | MAGE-C1 [777,796] | 11 | 1473 | FRQPIFSQYASHQPLPQATY | TEX15 |
| | | | | | | | [2742,2761] |
| 1 | 81 | ATVMASESLSVMSSNVSFSE | MAGE-C2 [354,373] | 11 | 1474 | PSSRILQLSKPKAPATLLEE | THEG [264,283] |
| 1 | 82 | EPVVPNAPPAYEKLSAEQSP | MART1 [94,113] | 11 | 1475 | DLKIQIEMEKKVVFSTISLG | TPTE [430,449] |
| 1 | 83 | RSQAGMFIYSNNRLIKMHEK | MORC1 [363,382] | 11 | 1476 | KVLKSERDKIFLLYEQAQEE | TSGA10 [55,74] |
| 1 | 84 | ETYNEYIYDLFVPVSSKFQK | MPHOSPH1 | 11 | 1477 | GAGEAPGALSTADPADQSVQ | TSP50 [39,58] |
| | | | [277,296] | | | | |
| 1 | 85 | GVSFFFLSFHISNLQFNSSL | MUC-1 [1039,1058] | 11 | 1478 | YEVEAYRRRHHNSSLNFFNW | TSPY1 [241,260] |
| 1 | 86 | VQPTQKQQKHRLFHWQANSE | NA17-A [45,64] | 11 | 1479 | YRNGDFFISSKELGYDYSYL | TYR [433,452] |
| 1 | 87 | EEAFSRASLVSVYNSYPYYP | NKX3.1 [202,221] | 11 | 1480 | ERRFSRSDQLKRHQRRHTGV | WT1 [361,380] |
| 1 | 88 | SLLRKKMLPEASLLIAIKPV | NLRP4 [263,282] | 11 | 1481 | FINHQIIYAGEKNHQYGKSF | ZNF165 |
| | | | | | | | [307,326] |
| 1 | 89 | IERLIYLYHKFHQLKVSNEE | NR6A1 [369,388] | 12 | 1482 | LADLSPFAFSGSNASVSAPS | 5T4 [154,173] |
| 1 | 90 | QSQGSVLAFTRTFIATPGSS | NXF2 [510,529] | 12 | 1483 | PKFHSESLSSNPSSFAPRVR | ACRBP |
| | | | | | | | [254,273] |
| 1 | 91 | KRASQYSGQLKVLIAENTML | NY-BR-1 | 12 | 1484 | WLGASVVAHLSTYQSEWMSR | ACTL8 |
| | | | [1103,1122] | | | | [338,357] |
| 1 | 92 | WTLSRFFSYLRSWDVDDLLL | NYD-TSPG [322,341] | 12 | 1485 | WRVLFLLSGLGGLRMDSNFD | ADAM2 [2,21] |
| 1 | 93 | KEFTVSGNILTIRLTAADHR | NY-ESO-1 [124,143] | 12 | 1486 | VIDNYLYIRYERNDSKLLED | ADAM29 |
| | | | | | | | [205,224] |
| 1 | 94 | DEKRELAKLRRTTNRILASS | ODF1 [100,119] | 12 | 1487 | KGEEELQKYIQESQALAKRS | AFP [396,415] |
| 1 | 95 | ADKDLYVAEALSTLESWRSR | ODF2 [428,447] | 12 | 1488 | QKATDIMDAMLRKLYNVMFA | AKAP-3 |
| | | | | | | | [368,387] |
| 1 | 96 | EKSTKYVETSAPSHSISART | ODF3 [109,128] | 12 | 1489 | LAKDLIVSALKLIQYHLTQQ | AKAP-4 |
| | | | | | | | [540,559] |
| 1 | 97 | NSPLPFQWRITHSFRWMAQV | ODF4 [63,82] | 12 | 1490 | VPHLQKVETRYKSYSPYDML | ANXA2 |
| | | | | | | | [222,241] |
| 1 | 98 | ADSVHLAWDLSRSLGAVVFS | OIP5 [88,107] | 12 | 1491 | YNPPDHEVVTMARMLQDVFE | BRDT [351,370] |
| 1 | 99 | MGFLRRLIYRRRPMIYVESS | PAGE1 [1,20] | 12 | 1492 | DQAPEVTLQADIEVMSTVHI | CABYR |
| | | | | | | | [274,293] |
| 1 | 100 | GPDMEAFQQELALLKIEDEP | PAGE2 [65,84] | 12 | 1493 | KEAQEQEFLSLQEEFQKLEK | CAGE1 |
| | | | | | | | [477,496] |
| 1 | 101 | RGDGQEAPDVVAFVAPGESQ | PAGE4 [12,31] | 12 | 1494 | SGKINRHEHYFNQFHRRNEL | CALR3 |
| | | | | | | | [365,384] |
| 1 | 102 | GTDVEAFQQELALLKIEDAP | PAGES [84,103] | 12 | 1495 | EKQDYKQKLKALKIEVNKLK | CCDC62 |
| | | | | | | | [204,223] |
| 1 | 103 | NHPVRFLQAQPIVPVQRAAE | PASD1 [599,618] | 12 | 1496 | EEQEEFKQLSDGIQELQQSL | CDCA1 |
| | | | | | | | [175,194] |
| 1 | 104 | RYKASQDPFPAAIILKVALN | PBK [130,149] | 12 | 1497 | ALQKKYLRMVVLAVYTNPED | CT46 [88,107] |
| 1 | 105 | KEMELWMKAMLDAALVQTEP | PEPP2 [254,273] | 12 | 1498 | LSGPAELRSFNMPSLDKMDG | CTAGE2 |
| | | | | | | | [603,622]; |
| | | | | | | | CTAGE5 |
| | | | | | | | [634,653] |
| 1 | 106 | QYAHKLAFLVGQSIHREPNL | PIWIL1 [833,852] | 12 | 1499 | AEAKMTFKIFQMNEERLKIA | CTAGE5 |
| | | | | | | | [188,207] |
| 1 | 107 | RSVVGFVASINLTLTKWYSR | PIWIL2 [755,774] | 12 | 1500 | FRQKQLLNAHFRKYHDANFI | CTCFL [523,542] |
| 1 | 108 | QKSIAGFVASTNAELTKWYS | PIWIL3 [663,682] | 12 | 1501 | QPFEENEFIDASRLVYDGVR | CTNNA2 |
| | | | | | | | [602,621] |
| 1 | 109 | RTLNKQGMMMSIATKIAMQM | PIWIL4 [580,599] | 12 | 1502 | GDYGMIDESIYFSSDVVTGN | CXorf48 [48,67] |
| 1 | 110 | KAVSQDMVIYSTEIHYSSKG | PLAC1 [85,104] | 12 | 1503 | SRDILNNFPHSIARQKRILV | CXorf61 [63,82] |
| 1 | 111 | RLDQLLRHVMNPLETLSITN | PRAME [312,331] | 12 | 1504 | AIKRNNPRKFLRSVGDGETV | DBPC [125,144] |
| 1 | 112 | KNTPGIYTSLVNYNLWIEKV | PRSS55 [279,298] | 12 | 1505 | GAVSLEGYFHLWKAENTLSK | DCAF12 |
| | | | | | | | [268,287] |
| 1 | 113 | SNPIVLRMMNDQLMFLERAF | PSMA [656,675] | 12 | 1506 | DAQESSLGLTGLQSLLQGFS | DKKL1 [37,56] |
| 1 | 114 | TKFKQSRAMNFDFPFKKGSG | RAGE-1 [227,246] | 12 | 1507 | GISHPIPLPSAAELLVKREN | DMRT1 |
| | | | | | | | [132,151] |
| 1 | 115 | GSTGFSSRLAATQDLPFIHQ | RCAS1 [123,142] | 12 | 1508 | LVISRKKRMAKYEKAEIKEM | EpCAM |
| | | | | | | | [286,305] |
| 1 | 116 | PPAFINMAATGVSSMSTREQ | SAGE1 [602,621] | 12 | 1509 | GVPFRTVSEWLESIKMQQYT | EPHA2 |
| | | | | | | | [903,922] |
| 1 | 117 | KDSITVFVSNLPYSMQEPDT | SART3 [701,720] | 12 | 1510 | MSEIRFTNLTWDQVITLDQV | FAM46D [1,20] |
| 1 | 118 | NNNIEKMITAFEELRVQAEN | SCP-1 [215,234] | 12 | 1511 | AVNRRLRALEEQGATWRHRE | FATE1 |
| | | | | | | | [143,162] |
| 1 | 119 | KLNQEYSQQFLTLFQQWELD | SCP3a [120,139] | 12 | 1512 | VESAVWYVKKFGRYLEHLEV | FBXO39 [75,94] |
| 1 | 120 | RRHELYKAHEWARAIIESDQ | SCRN1 [343,362] | 12 | 1513 | KHNQDFIKRNIELAKESRNP | FSIP1 [249,268] |
| 1 | 121 | TSELKTEGVSPYLMLIRLRK | se57-1 [316,335] | 12 | 1514 | TSYLYLSMAFYFNREDVALE | FTHL17 [31,50] |
| 1 | 122 | QSSGISFQSKYLSFFILGQT | SLCO6A1 [218,237] | 12 | 1515 | SKDQQKILAALKELQVEEIQ | GASZ [320,339] |
| 1 | 123 | KERQLSTMITQLISLREQLL | SOX-6 [187,206] | 12 | 1516 | GPLSVYPPASSSSLSGGHAS | GATA-3 |
| | | | | | | | [128,147] |
| 1 | 124 | PDNIPAFAAAYFESLLEKRE | SP17 [32,51] | 12 | 1517 | EELVNGMYRIYDMENVLLGL | Glypican-3 |
| | | | | | | | [304,323] |
| 1 | 125 | EKBPSLVYQHLLYLSKAERF | SPAG1 [863,882] | 12 | 1518 | QVSLKVSNDGPTLIGANASF | gp100 [65,84] |
| 1 | 126 | RDAVKFFVAVPGQVISPQSS | SPAG9 [1223,1242] | 12 | 1519 | TRNDWRVASELINILERANQ | HAGE [592,611] |
| 1 | 127 | KKMKTSESSTILVVRYRRNV | SPAN-Xc [40,59] | 12 | 1520 | QLSTGGSVASAVKLNKQQTD | HDAC1 |
| | | | | | | | [111,130] |
| 1 | 128 | TRIQFIRWSHTRIFQVPSEM | SPATA19 [112,131] | 12 | 1521 | SDEYIKFLRSIRPENMSEYS | HDAC2 [70,89] |
| 1 | 129 | AEIQALTFLSSDYLSRVYLP | SPO11 [368,387] | 12 | 1522 | EDYIDFLQRVSPTNMQGFTK | HDAC3 [64,83] |
| 1 | 130 | RIQVEHPQMTFGRLHRIIPK | SSX-1 [91,110] | 12 | 1523 | TESVSHFSDLGQSFSFHSGN | IGFS11 |
| | | | | | | | [332,351] |
| 1 | 131 | VERPQMTFGRLQGISPKIMP | SSX-2 [94,113] | 12 | 1524 | LVTATVENETYTLKTTNETR | IL13RA2 |
| | | | | | | | [283,302] |
| 1 | 132 | QRPQMTFGRLQGIFPKIMPK | SSX-3 [95,114] | 12 | 1525 | PARRAKRMRAEAMNIKIEPE | JARID1B |
| | | | | | | | [227,246] |
| 1 | 133 | SEKIVYVYMKLNYEVMTKLG | SSX-4 [43,62] | 12 | 1526 | KKIRESYENDIASMNLQAHL | KOC1 [345,364] |
| 1 | 134 | NQVEHPQMTFGRLQGIFPKI | SSX-5 [92,111] | 12 | 1527 | KSDNEEVREAAALALANLTT | KU-CT-1 |
| | | | | | | | [366,385] |
| 1 | 135 | EFEETAKKVRRAIEQLAAMD | Survivin [123,142] | 12 | 1528 | AIGLSVMDLVGSILKNLRRV | LDHC [251,270] |
| 1 | 136 | LFLRSQEAAATVQLFQEEHR | SYCE1 [229,248] | 12 | 1529 | KPPYSRLDYTDAKFVDVIHS | LIPI [213,232] |
| 1 | 137 | ESGQYRANEGTSSIVMEIQK | TAF7L [377,396] | 12 | 1530 | IFGKASESLQLVFGIDVKEA | MAGE-A1 |
| | | | | | | | [143,162] |
| 1 | 138 | GADAQYFVYSNESVRPYTPF | TAG-1 [765,784] | 12 | 1531 | SSPSVVASLPLDQSDEGSSS | MAGE-A10 |
| | | | | | | | [92,111] |
| 1 | 139 | RGLPASTLSRLSNRLLLRLE | TAG-2a [35,54] | 12 | 1532 | QNWVQEKYLVYRQVPGTDPA | MAGE-A11 |
| | | | | | | | [361,380] |
| 1 | 140 | SSEVLEYMNQLSASLKETYA | TDRD1 [285,304] | 12 | 1533 | QEGPSTFPDLETSFQVALSR | MAGE-A12 |
| | | | | | | | [92,111] |
| 1 | 141 | LPGYRYLNSWRPSLFYKIAN | TEKT5 [41,60] | 12 | 1534 | DLESEFQAAISRKMVELVHF | MAGE-A2 |
| | | | | | | | [100,119] |
| 1 | 142 | LSQFWEFSETTASTVSTTLH | TEX101 [120,139] | 12 | 1535 | VGNWQYFFPVIFSKASSSLQ | MAGE-A3 |
| | | | | | | | [140,159] |
| 1 | 143 | RLLKAGVISAQNIYSFGFGK | TEX14 [193,212] | 12 | 1536 | LEHVVRVNARVRIAYPSLRE | MAGE-A4 |
| | | | | | | | [288,307] |
| 1 | 144 | GKINQNYASIITEAFPKPKD | TEX15 [368,387] | 12 | 1537 | VGNWQYFFPVIFSKASDSLQ | MAGE-A6 |
| | | | | | | | [140,159] |
| 1 | 145 | VSRAAQMAVPSSRILQLSKP | THEG [255,274] | 12 | 1538 | AASSSSTLIMGTLEEVTDSG | MAGE-A8 |
| | | | | | | | [38,57] |
| 1 | 146 | SSERTINLLEVLPWPTEWIF | TMEM31 [63,82] | 12 | 1539 | EEPSSSVDPAQLEFMFQEAL | MAGE-A9 |
| | | | | | | | [89,108] |
| 1 | 147 | DLDLTYVTERIIAMSFPSSG | TPTE [236,255] | 12 | 1540 | ETTKMKVLEFLAKMNGATPR | MAGE-B1 |
| | | | | | | | [279,298] |
| 1 | 148 | RKDQGFLEKEFYHKTNIKMR | TRAG-3 [14,33] | 12 | 1541 | AETSKMKVLEFLAKVNGTTP | MAGE-B2 |
| | | | | | | | [281,300] |
| 1 | 149 | EELQKVQFEKVSALADLSST | TSGA10 [492,511] | 12 | 1542 | LKKPQRALSTTTSVDVSYKK | MAGE-B3 |
| | | | | | | | [62,81] |
| 1 | 150 | VIMHSRYRAQRFWSWVGQAN | TSP50 [186,205] | 12 | 1543 | TTAMTSAYSRATSSSSSQPM | MAGE-B4 |
| | | | | | | | [327,346] |
| 1 | 151 | KIMLFFRSNPYFQNKVITKE | TSPY1 [200,219] | 12 | 1544 | RTSQHLVVAFGVELKEMESS | MAGE-B6 |
| | | | | | | | [239,258] |
| 1 | 152 | HNRESYMVPFIPLYRNGDFF | TYR [420,439] | 12 | 1545 | PLQSPVISFSSSTSLSPFSE | MAGE-C1 |
| | | | | | | | [850,869] |
| 1 | 153 | RGKKGAATKMAAVTAPEAES | VCX [50,69] | 12 | 1546 | EEEEEASSASSTLYLVFSPS | MAGE-C2 |
| | | | | | | | [34,53] |
| 1 | 154 | NKRYFKLSHLQMHSRKHTGE | WT1 [331,350] | 12 | 1547 | ERSQRSQIANITTVWRAQPT | MORC1 |
| | | | | | | | [668,687] |
| 1 | 155 | VGILHLGSRQKKIRIQLRSQ | XAGE-1 [13,32]; | 12 | 1548 | VQQIQSNYDIATAELHVQKS | MPHOSPH1 |
| | | | XAGE-1b [13,32]; | | | | [933,952] |
| | | | XAGE-1c [92,111] | | | | |
| 1 | 156 | SRQKKIRIQLRSQVLGREMR | XAGE-1d [20,39] | 12 | 1549 | DVSVSEVPFPFSAQSGAGVP | MUC-1 |
| | | | | | | | [1138,1157] |
| 1 | 157 | MSWRGRSTYRPRPRRSLQPP | XAGE-2 [1,20] | 12 | 1550 | LSSELGELEKHSSLPALKEE | NKX3.1 |
| | | | | | | | [184,203] |
| 1 | 158 | MIWRGRSTYRPRPRRSVPPP | XAGE-3 [1,20] | 12 | 1551 | YISEMLLRNKSVRYLDLSAN | NLRP4 |
| | | | | | | | [798,817] |
| 1 | 159 | KSFKSPKLAKHAAVFSGEKT | ZNF165 [324,343] | 12 | 1552 | EELHRFSDEGMEVIERLIYL | NR6A1 |
| | | | | | | | [356,375] |
| 2 | 160 | ELASNHFLYLPREVLAQLPS | 5T4 [216,235] | 12 | 1553 | VTIPYGIKYDKAWLMNSIQS | NXF2 [127,146] |
| 2 | 161 | SHKTPFVSPLLASQSLSIGN | ACRBP [399,418] | 12 | 1554 | ESLDQKLFQLQSKNMWLQQQ | NY-BR-1 |
| | | | | | | | [1261,1280] |
| 2 | 162 | NFSVWLGASVVAHLSTYQSE | ACTL8 [334,353] | 12 | 1555 | YWRTSSFRMTEHNSVKPWQQ | NYD-TSPG |
| | | | | | | | [124,143] |
| 2 | 163 | EANELLHTFLRWKTSYLVLR | ADAM2 [242,261] | 12 | 1556 | ELSKSMESMRGHLQAQLRSK | ODF2 [337,356] |
| 2 | 164 | IKVKKLLFSKHLPVFTYTDQ | ADAM29 [67,86] | 12 | 1557 | PINIWIFELERNVSIPIGWS | ODF4 [163,182] |
| 2 | 165 | SGEKNIFLASFVHEYSRRHP | AFP [344,363] | 12 | 1558 | GGTERAIDQASFTTSMEWDT | OIP5 [22,41] |
| 2 | 166 | SEGIMTYANSVVSEMMVSIM | AKAP-3 [282,301] | 12 | 1559 | GPPDPQAFQGPAAYQPDQMR | PASD1 |
| | | | | | | | [735,754] |
| 2 | 167 | DQVNIDYLMNRPQNLRLEMT | AKAP-4 [161,180] | 12 | 1560 | PINMEELDESYQKVIELFSV | PBK [280,299] |
| 2 | 168 | STPPSAYGSVKAYTNETAER | ANXA2 [18,37] | 12 | 1561 | YSPQRTYRSEVSSPIQRGDV | PEPP2 |
| | | | | | | | [556,575] |
| 2 | 169 | QLLQARLMKEESPVVSWRLE | BAGE-1 [13,32]; | 12 | 1562 | APLISVKPLENWLLIYTRRN | PIWIL1 |
| | | | BAGE-2 [13,32]; | | | | [485,504] |
| | | | BAGE-3 [13,32]; | | | | |
| | | | BAGE-5 [13,32] | | | | |
| 2 | 170 | TALDVHFVSTLEPLSNAVKR | BAGE-2 [37,56]; | 12 | 1563 | DIPLKQLMVIGMDVYHDPSR | PIWIL2 |
| | | | BAGE-3 [37,56] | | | | [733,752] |
| 2 | 171 | KEMLAKKHFSYAWPFYNPVD | BRDT [281,300] | 12 | 1564 | KRINTRFFLKHGSNFQNPPP | PIWIL3 |
| | | | | | | | [763,782] |
| 2 | 172 | AKYSSVYMEAEATALLSETS | CABYR [372,391] | 12 | 1565 | DYPKIIKVQENPAAFVRAIQ | PIWIL4 |
| | | | | | | | [518,537] |
| 2 | 173 | ENHPKSMTMMPALFKENRND | CAGE1 [756,775] | 12 | 1566 | DVMHLSQSPSVSQLSVLSLS | PRAME |
| | | | | | | | [338,357] |
| 2 | 174 | FSNKGKTLVIQYTVKHEQKM | CALR3 [84,103] | 12 | 1567 | FKRANMENDIALLLLASPIK | PRSS55 |
| | | | | | | | [148,167] |
| 2 | 175 | TVFGEKSVITLSSIFTKELV | CCDC62 [385,404] | 12 | 1568 | PRRTILFASWDAEEFGLLGS | PSMA [412,431] |
| 2 | 176 | TLSFPRYNVAEIVIHIRNKI | CDCA1 [3,22] | 12 | 1569 | IGEGTFSEVMKMQSLREGNY | RAGE-1 [10,29] |
| 2 | 177 | PTSWSSAIQSWYDEILDFVY | CR1SP2 [104,123] | 12 | 1570 | RKQENVLSNVLSGLINMAGA | SAGE1 |
| | | | | | | | [451,470] |
| 2 | 178 | SPSYQKRQRMALLARKQGAG | CT45 [24,43] | 12 | 1571 | PIPESVIQNYNKALQQLEKY | SART3 |
| | | | | | | | [276,295] |
| 2 | 179 | DALQKKYLRMVVLAVYTNPE | CT46 [87,106] | 12 | 1572 | FVPPRSSSSQVSAVKPQTLG | SCP-1 [10,29] |
| 2 | 180 | EEGNEAANFDLAVVARRYPA | CT47 [97,116] | 12 | 1573 | RKRLEMYTKASLKTSNQKIE | SCP3a [89,108] |
| 2 | 181 | FLFGGNNFIQNFYLPQNYID | CTAGE1 [18,37] | 12 | 1574 | RPRDEVQEVVYFSAADHEPE | SCRN1 [30,49] |
| 2 | 182 | ALKKLIHAAKLNASLKTLEG | CTAGE2 [282,301]; | 12 | 1575 | NLVQRMEKEKRTLLERKLSL | se57-1 |
| | | | CTAGE5 [312,331] | | | | [193,212] |
| 2 | 183 | MFTSSRMSSFNRHMKTHTSE | CTCFL [263,282] | 12 | 1576 | IRFGGFRKRKKAKSSVSKKP | SLCO6A1 |
| | | | | | | | [59,78] |
| 2 | 184 | SSESSMLDSATSLIQAAKNL | CTNNA2 [853,872] | 12 | 1577 | QAFPDMHNSNISKILGSRWK | SOX-6 [641,660] |
| 2 | 185 | RNGVIDYTIFFTLDSVKLPD | CXorf48 [188,207] | 12 | 1578 | EKHLQALAPESRALRKDKPA | SPAG1 [62,81] |
| 2 | 186 | DLSRDILNNETHSIARQKRI | CXorf61 [61,80] | 12 | 1579 | GLAGSIYREFERLIGRYDEE | SPAG9 [26,45] |
| 2 | 187 | DEPVLAIQVLGTVKWENVRN | DBPC [86,105] | 12 | 1580 | IQPIITSLARNEAPAFTIDN | SPO11 [55,74] |
| 2 | 188 | GQGSLLEYDIRAQRFLEERL | DCAF12 [358,377] | 12 | 1581 | KGERPGAAHQAGPDVLIGQE | SYCE1 [313,332] |
| 2 | 189 | TELHPRVAFWIIKLPRRRSH | DKKL1 [161,180] | 12 | 1582 | VIESLRTLEKKTFYKTADIS | TAF7L [159,178] |
| 2 | 190 | SESTYYSSFYQPSLEPYYNN | DMRT1 [205,224] | 12 | 1583 | PGNISWTESSSSLSIKWDPV | TAG-1 |
| | | | | | | | [916,935] |
| 2 | 191 | MLASWARIAARAVQPKALNS | DPPA2 [185,204] | 12 | 1584 | SLSISNPGLFTSLGPPLRST | TDRD1 |
| | | | | | | | [149,168] |
| 2 | 192 | PGQTLIYYVDEKAPEFSMQG | EpCAM [244,263] | 12 | 1585 | QFTETNLAFNARISEVTDVK | TEKT5 [334,353] |
| 2 | 193 | DLAPDTTYLVQVQALTQEGQ | EPHA2 [496,515] | 12 | 1586 | KILKKGIYVDAVNSLGQTAL | TEM14 [41,60] |
| 2 | 194 | EKEKEVRSLSKEREESYPED | FAM133A [168,187] | 12 | 1587 | SNAAIQIASATMPALSLNNE | TEX15 |
| | | | | | | | [598,617] |
| 2 | 195 | KNLELKEVSSLRRQFEFSVD | FAM46D [155,174] | 12 | 1588 | SRRVEELSRPKRFYLEYYNN | THEG [191,210] |
| 2 | 196 | EVMRRQLYAVNRRLRALEEQ | FATE1 [135,154] | 12 | 1589 | SSGRQSFYRNPIKEVVRFLE | TPTE [253,272] |
| 2 | 197 | NLEVNEFFERIMEYERLARI | FBXO39 [282,301] | 12 | 1590 | TERDVAFTELRRMTTERDSL | TSGA10 |
| | | | | | | | [103,122] |
| 2 | 198 | QSLEEDSALEALLNFFFPTT | FMR1NB [114,133] | 12 | 1591 | FWSWVGQANDIGLLELKQEL | TSP50 [197,216] |
| 2 | 199 | KLQELSAASPTISSFSPRLE | FSIP1 [326,345] | 12 | 1592 | KVITKEYLVNITEYRASHST | TSPY1 [214,233] |
| 2 | 200 | LELYTSYLYLSMAFYFNREE | FTHL17 [27,46] | 12 | 1593 | ASPLTGIADASQSSMHNALH | TYR [348,367] |
| 2 | 201 | MSWRGRSTYRPRPRRYVEPP | GAGE-1 [1,20]; | 12 | 1594 | AQFPNHSFEHEDPMGQQGSL | WT1 [169,188] |
| | | | GAGE-2 [1,20]; | | | | |
| | | | GAGE-8 [1,20] | | | | |
| 2 | 202 | MNLSRGKSTYYWPRPRRYVQ | GAGE-3 [1,20] | 12 | 1595 | DQSFEWNSDFINHQIIYAGE | ZNF165 |
| | | | | | | | [298,317] |
| 2 | 203 | MSWRGRSTYYWPRPRRYVQP | GAGE-6 [1,20]; | 13 | 1596 | GYHYRYEINADPRLTNLSSN | 5T4 [398,417] |
| | | | GAGE-7 [1,20] | | | | |
| 2 | 204 | DREKDHIFSSYTAFGELEVF | GASZ [263,282] | 13 | 1597 | SGWLQTEFLSFQDGDEPTKI | ACRBP |
| | | | | | | | [457,476] |
| 2 | 205 | GLYYKLHNINRPLTMKKEGI | GATA-3 [343,362] | 13 | 1598 | GSRVELTPMQRVAPEMFFSP | ACTL8 |
| | | | | | | | [243,262] |
| 2 | 206 | ELIQKLKSFISFYSALPGYI | Glypican-3 | 13 | 1599 | VNLMQKNFLPHNERVYSYSG | ADAM2 [58,77] |
| | | | [390,409] | | | | |
| 2 | 207 | VLMAVVLASLIYRRRLMKQD | gp100 [605,624] | 13 | 1600 | FQGILQINDFAYEIKPLAFS | ADAM29 |
| | | | | | | | [123,142] |
| 2 | 208 | SATWPHSVHRLAQSYLKEPM | HAGE [427,446] | 13 | 1601 | LIFLLNFTESRTLHRNEYGI | AFP [9,28] |
| 2 | 209 | GAGEGKYYAVNYPLREGIDD | HDAC1 [215,234] | 13 | 1602 | PPSEKSFFYKEVFESRNGDY | AKAP-3 |
| | | | | | | | [234,253] |
| 2 | 210 | GAGEGKYYAVNFPMRDGIDE | HDAC2 [216,235] | 13 | 1603 | SSGKPIPASVVLKRVLLRHT | AKAP-4 |
| | | | | | | | [356,375] |
| 2 | 211 | QEAFYLTDRVMTVSFHEYGN | HDAC3 [178,197] | 13 | 1604 | VNILTNRSNAQRQDIAFAYQ | ANXA2 [57,76] |
| 2 | 212 | MASFRELTLSEEVPPNHPSR | HOM-TES-85 [1,20] | 13 | 1605 | PESQQQYNVVETVEVTEQLR | BRDT [255,274] |
| 2 | 213 | QVQGTVTIRNISALSSGLYQ | IGFS11 [195,214] | 13 | 1606 | KTTSGMSEKSVESVKLAQLE | CABYR |
| | | | | | | | [350,369] |
| 2 | 214 | QLQNIVKPLPPVYLTFTRES | IL13RA2 [231,250] | 13 | 1607 | SNLYLEKRVKELQMKITKQQ | CAGE1 |
| | | | | | | | [329,348] |
| 2 | 215 | RGHYERILNPYNLELSGDSL | JARID1B [172,191] | 13 | 1608 | KMKNITYLTQYDLSEFENIG | CALR3 |
| | | | | | | | [279,298] |
| 2 | 216 | YENDIASMNLQAHLIPGLNL | KOC1 [351,370] | 13 | 1609 | APGHMSDVEWMSIFKPSKMQ | CCDC62 |
| | | | | | | | [525,544] |
| 2 | 217 | ATVLTNMAMQEPLRLNIQNH | KU-CT-1 [419,438] | 13 | 1610 | RYNVAEIVIHIRNKILTGAE | CDCA1 [8,27] |
| 2 | 218 | GAVLEDFTVSGNLLEMSVRD | Lage-1 [120,139] | 13 | 1611 | KTRSGKVFQNKMANGNQPVK | CT46 [333,352] |
| 2 | 219 | WAIGLSVMDLVGSILKNLRR | LDHC [250,269] | 13 | 1612 | SETRASLYPPTLLEGPLRLS | CTAGE2 |
| | | | | | | | [498,517] |
| 2 | 220 | LPSTRKLYEKKLVQLLVSPP | LEMD1 [27,46] | 13 | 1613 | SETRAFLSPPTLLEGPLRLS | CTAGE5 |
| | | | | | | | [529,548] |
| 2 | 221 | AQFYNDEVNISSIGLTYFQS | LIPI [409,428] | 13 | 1614 | QLLAERTKEQLFFVETMSGE | CTCFL [190,209] |
| 2 | 222 | VSARVRFFFPSLREAALREE | MAGE-A1 [286,305] | 13 | 1615 | GAIRGRAARVIHIINAEMEN | CTNNA2 |
| | | | | | | | [541,560] |
| 2 | 223 | LESVIRNYEDHFPLLFSEAS | MAGE-A10 | 13 | 1616 | GLINSNTENNLAVYDLSRDI | CXorf61 [47,66] |
| | | | [161,180] | | | | |
| 2 | 224 | YAGREHFLFGEPKRLLTQNW | MAGE-A11 | 13 | 1617 | LHKRKRLPPVKRSLVYYLKN | DCAF12 [33,52] |
| | | | [344,363] | | | | |
| 2 | 225 | SVIRNFQDFFPVIFSKASEY | MAGE-A12 | 13 | 1618 | LFSAPMDFRGLPGNYHKEEN | DKKL1 [71,90] |
| | | | [138,157] | | | | |
| 2 | 226 | PDLESEFQAAISRKMVELVH | MAGE-A2 [99,118] | 13 | 1619 | VENTPDLVSDSTYYSSFYQP | DMRT1 |
| | | | | | | | [197,216] |
| 2 | 227 | VVGNWQYFFPVIFSKASSSL | MAGE-A3 [139,158] | 13 | 1620 | YQLDPKFITSILYENNVITI | EpCAM |
| | | | | | | | [174,193] |
| 2 | 228 | ETSYVKVLEHVVRVNARVRI | MAGE-A4 [281,300]; | 13 | 1621 | AGMKYLANMNYVHRDLAARN | EPHA2 |
| | | | MAGE-A8 [283,302] | | | | [725,744] |
| 2 | 229 | ESVFRAALSKKVADLIHFLL | MAGE-AS [102,121] | 13 | 1622 | WSLISLSNNTGKNLELKFVS | FAM46D |
| | | | | | | | [144,163] |
| 2 | 230 | VVGNWQYFFPVIFSKASDSL | MAGE-A6 [139,158] | 13 | 1623 | KAAGSASAKRVWNMTATRPK | FATE1 [57,76] |
| 2 | 231 | DGREHSVYWKLRKLLTQEWV | MAGE-A8 [235,254] | 13 | 1624 | FYFKIWAFLDVSFVERILKS | FBXO39 |
| | | | | | | | [369,388] |
| 2 | 232 | ANAETSYEKVINYLVMLNAR | MAGE-A9 [276,295] | 13 | 1625 | SKTLGIGRLKRPSFLIMPLY | FSIP1 [524,543] |
| 2 | 233 | TEEEIWKFMNVLGAYDGEEH | MAGE-B1 [216,235] | 13 | 1626 | EHMTDLLKERDITLRHLLTM | GASZ [289,308] |
| 2 | 234 | QFLWGPRAYAETSKMKVLEF | MAGE-B2 [272,291] | 13 | 1627 | YGNSVRATVQRYPPTHHGSQ | GATA-3 [64,83] |
| 2 | 235 | TNKKKVSFSSPLILGATIQK | MAGE-B3 [33,52] | 13 | 1628 | SLQVTRIFLQALNLGIEVIN | Glypican-3 |
| | | | | | | | [222,241] |
| 2 | 236 | SLTRKTKMLVQFLLYKYKMK | MAGE-B4 [108,127] | 13 | 1629 | VLPDGQVIWVNNTIINGSQV | gp100 [96,115] |
| 2 | 237 | GPRAYAETTKMRVLRVLABS | MAGE-B6 [360,379] | 13 | 1630 | ALKSHFVGAVIGRGGSKIKN | HAGE [73,92] |
| 2 | 238 | NPASSFFSSALLSIFQSSPE | MAGE-C1 [130,149] | 13 | 1631 | SEDYIKFLRSIRPDNMSEYS | HDAC1 [69,88] |
| 2 | 239 | SSASSTLYLVFSPSSFSTSS | MAGE-C2 [40,59] | 13 | 1632 | QLSTGGSVAGAVKLNRQQTD | HDAC2 |
| | | | | | | | [112,131] |
| 2 | 240 | NAPPAYEKLSAEQSPPPYSP | MART1 [99,118] | 13 | 1633 | NHAPSVQIHDVPADLLTYDR | HDAC3 |
| | | | | | | | [370,389] |
| 2 | 241 | DEPQKFAMELSIIYKYSPFK | MORC1 [159,178] | 13 | 1634 | QPEQVILYQGGQMETGAPRF | IGFS11 [69,88] |
| 2 | 242 | YEQANLNMANSIKFSVWVSF | MPHOSPH1 | 13 | 1635 | QHRAQQLLSSGNLKFVQDRV | JARID1B |
| | | | [256,275] | | | | [1263,1282] |
| 2 | 243 | NIKFRPGSVVVQLTLAFREG | MUC-1 [1091,1110] | 13 | 1636 | LENFTLKVAYIPDEMAAQQN | KOC1 [144,163] |
| 2 | 244 | HRLFHWQANSERADIPASLR | NA17-A [54,73] | 13 | 1637 | AAEADGIDPLINLLSSKREG | KU-CT-1 |
| | | | | | | | [394,413] |
| 2 | 245 | PQKRSRAAFSHTQVIELERK | NKX3.1 [123,142] | 13 | 1638 | LKELADELALVDVALDKLKG | LDHC [41,60] |
| 2 | 246 | GINNVSFSGQSVLLFEVLFY | NLRP4 [673,692] | 13 | 1639 | DMNVIVVEWSRGATTFIYNR | LIPI [126,145] |
| 2 | 247 | IPHLFSYSGHSPLLPQQARS | NR6A1 [226,245] | 13 | 1640 | GEPRKLLTQDLVQEKYLEYR | MAGE-A1 |
| | | | | | | | [233,252] |
| 2 | 248 | QEMVQAFSAQSGMKLEWSQK | NXF2 [573,592] | 13 | 1641 | QSETQGLEGAQAPLAVEEDA | MAGE-A10 |
| | | | | | | | [19,38] |
| 2 | 249 | SVVAKLLSHGAVIEVHNKAS | NY-BR-1 [97,116] | 13 | 1642 | LLLRKYRVKGLITKAEMLGS | MAGE-A11 |
| | | | | | | | [232,251] |
| 2 | 250 | NAEDWNLYWRTSSFRMTEHN | NYD-TSPG [117,136] | 13 | 1643 | TKAEMLGSVIRNFQDFFPVI | MAGE-A12 |
| | | | | | | | [131,150] |
| 2 | 251 | AMPFATPMEAELARRSLAQD | NY-ESO-1 [93,112] | 13 | 1644 | KPEEGLEARGEALGLVGAQA | MAGE-A2 |
| | | | | | | | [11,30] |
| 2 | 252 | LRPSLRSLERKAIRAIEDEK | ODF1 [83,102] | 13 | 1645 | EEGPSTFPDLESEFQAALSR | MAGE-A3 |
| | | | | | | | [92,111]; |
| | | | | | | | MAGE-A6 |
| | | | | | | | [92,111] |
| 2 | 253 | GDGPYSTFLTSSPIRSRSPP | ODF2 [809,828] | 13 | 1646 | EEGVEAQEEALGLVGAQAPT | MAGE-A4 |
| | | | | | | | [13,32] |
| 2 | 254 | DVRVTKFKAPQYTMAARVEP | ODF3 [189,208] | 13 | 1647 | PAHLESLFREALDEKVAELV | MAGE-A8 |
| | | | | | | | [101,120] |
| 2 | 255 | LGQDGRLLSSTLSLSSNRSL | ODF4 [39,58] | 13 | 1648 | KEPVTKAEMLESVIKNYKRY | MAGE-A9 |
| | | | | | | | [126,145] |
| 2 | 256 | RSLGAVVFSRVTNNVVLEAP | OIP5 [99,118] | 13 | 1649 | GEPRKFITQDLVQEKYLKYE | MAGE-B1 |
| | | | | | | | [239,258] |
| 2 | 257 | NDQESSQPVGSVIVQEPTEE | PAGE2 [16,35] | 13 | 1650 | GEPWKLITKELVQEKYLEYK | MAGE-B2 |
| | | | | | | | [242,261] |
| 2 | 258 | MSARVRSRSRGRGDGQEAPD | PAGE4 [1,20] | 13 | 1651 | QSRTDPLIMKTNMLVQFLME | MAGE-B3 |
| | | | | | | | [105,124] |
| 2 | 259 | RGNDQESSQPVGPVIVQQPT | PAGE5 [33,52] | 13 | 1652 | AREEEIWEFLNMLGIYDGKR | MAGE-B4 |
| | | | | | | | [215,234] |
| 2 | 260 | SSQRKLNWIPSEPTYDYFNQ | PASD1 [16,35] | 13 | 1653 | TKMRVLRVLADSSNTSPGLY | MAGE-B6 |
| | | | | | | | [368,387] |
| 2 | 261 | LTLWEMMTLSIPHINLSNED | PBK [235,254] | 13 | 1654 | STFEGFAQSSLQIPVSPSFS | MAGE-C1 |
| | | | | | | | [258,277] |
| 2 | 262 | GWEEAYTFEGARYYINHNER | PEPP2 [61,80] | 13 | 1655 | SEEVIWEVLNAVGVYAGREH | MAGE-C2 |
| | | | | | | | [245,264] |
| 2 | 263 | NSLIQNLFKVTPAMGMQMRK | PIWIL1 [509,528] | 13 | 1656 | KLREILLYFFPEHQLPSELE | MORC1 |
| | | | | | | | [824,843] |
| 2 | 264 | LVGRNEYDPTSAMVLQQHRL | PIWIL2 [338,357] | 13 | 1657 | QQHVPFRESKLTHYFQSFFN | MPHOSPH1 |
| | | | | | | | [426,445] |
| 2 | 265 | LRYYNILFRRTFKLLEFEQV | PIWIL3 [222,241] | 13 | 1658 | ETQFNQYKTEAASRYNLTIS | MUC-1 |
| | | | | | | | [1118,1137] |
| 2 | 266 | YIPDLASRRLRIALLYSHSE | PIWIL4 [130,149] | 13 | 1659 | TTLLMKLMMAWSDNKIFRER | NLRP4 |
| | | | | | | | [162,181] |
| 2 | 267 | GIRAKAVSQDMVIYSTEIHY | PLAC1 [81,100] | 13 | 1660 | LTVYSKQIFGELADVTAKYS | NR6A1 |
| | | | | | | | [333,352] |
| 2 | 268 | SPEKEEQYIAQFTSQFLSLQ | PRAME [277,296] | 13 | 1661 | MAATLKIIERNFPELLSLNL | NXF2 [258,277] |
| 2 | 269 | DFKRANMENDIALLLLASPI | PRSSSS [147,166] | 13 | 1662 | GFHHIHEQIMEYIRKLSKNH | NY-BR-1 |
| | | | | | | | [193,212] |
| 2 | 270 | QSGAAVVHEIVRSFGTLKKE | PSMA [389,408] | 13 | 1663 | DENLKPWLLEVNYSPALTLD | NYD-TSPG |
| | | | | | | | [377,396] |
| 2 | 271 | VVRLSSYSSPTLQSVLGSGT | RAGE-1 [357,376] | 13 | 1664 | ELERKLEATSAQNIEFLQVI | ODF2 [668,687] |
| 2 | 272 | QLEPDYFKEMTPTIRKTQKI | RCAS1 [89,108] | 13 | 1665 | NRSLGQRQNSPLPFQWRITH | ODF4 [55,74] |
| 2 | 273 | RQFVEFTIKEAARFKKVVLI | SAGE1 [860,879] | 13 | 1666 | PSSPVAYDIISQELELMKKL | PASD1 |
| | | | | | | | [361,380] |
| 2 | 274 | KELEELRAAFTRALEYLKQE | SART3 [440,459] | 13 | 1667 | NDLIEERYKASQDPFPAAII | PBK [124,143] |
| 2 | 275 | QHKIAEMVALMEKHKHQYDK | SCP-1 [704,723] | 13 | 1668 | IPTSPSHGSIAAYQGYSPQR | PEPP2 |
| | | | | | | | [541,560] |
| 2 | 276 | METQQQEIASVRKSLQSMLF | SCP3a [217,236] | 13 | 1669 | KVLRSETVLDFMENFYHQTE | PIWIL1 |
| | | | | | | | [272,291] |
| 2 | 277 | DSEFFLTTASGVSVLPQNRS | SCRN1 [268,287] | 13 | 1670 | SQVVNVRTIGQPTRLRSVAQ | PIWIL2 |
| | | | | | | | [696,715] |
| 2 | 278 | EISILQEQISHLQFVIHSQH | se57-1 [235,254] | 13 | 1671 | LLANHFRVISRPQWVAYKYN | PIWIL3 |
| | | | | | | | [127,146] |
| 2 | 279 | KKHRYLRLLPEALIREGGFR | SLCO6A1 [46,65] | 13 | 1672 | QYDITVSDLNQPMLVSLLKK | PIWIL4 |
| | | | | | | | [343,362] |
| 2 | 280 | SEPHIKRPMNAFMVWAKDER | SOX-6 [617,636] | 13 | 1673 | RRLWGSIQSRYISMSVWTSP | PRAME [4,23] |
| 2 | 281 | KEEVAAVKIQAAFRGHIARE | SP17 [113,132] | 13 | 1674 | VTQLEGRPFNAEKRRTSVKQ | PRSS55 |
| | | | | | | | [298,317] |
| 2 | 282 | YYTRSISALPTVVAYNNRAQ | SPAO1 [231,250] | 13 | 1675 | RLQDFDKSNPIVLRMMNDQL | PSMA [649,668] |
| 2 | 283 | REEAQKMSSLLPTMWLGAQN | SPAG9 [959,978] | 13 | 1676 | KLKLSGVVRLSSYSSPTLQS | RAGE-1 |
| | | | | | | | [351,370] |
| 2 | 284 | NPLQMEEEEFMEIMVEIPAK | SPAN-Xc [78,97] | 13 | 1677 | NNQPQPSYDLSTVLPGLTYL | SAGE1 |
| | | | | | | | [542,561] |
| 2 | 285 | KGVGLPFLPITSSDIDVVES | SPATA19 [14,33] | 13 | 1678 | FRYSTSLEKHKLFISGLPFS | SART3 |
| | | | | | | | [792,811] |
| 2 | 286 | KSAQKFSLILKILSMIYKLV | SPO11 [106,125] | 13 | 1679 | MEKQKPFALFVPPRSSSSQV | SCP-1 [1,20] |
| 2 | 287 | EKISYVYMKRNYKAMTKLGF | SSX-1 [44,63] | 13 | 1680 | LQQSRIVQSQRLKTIKQLYE | SCP3a [161,180] |
| 2 | 288 | EWEKMKASEKIFYVYMKRKY | SSX-2 [36,55] | 13 | 1681 | TGEGEFNESEVESPVEDHLD | SCRN1 |
| | | | | | | | [215,234] |
| 2 | 289 | EKIVYVYMKRKYEAMTKLGF | SSX-3 [44,63] | 13 | 1682 | KQEDSKQLLQVNKLEKEQKL | se57-1 |
| | | | | | | | [148,167] |
| 2 | 290 | VERPQMTFGSLQRIFPKIMP | SSX-4 [94,113] | 13 | 1683 | LLPEALIREGGERKRKKAKS | SLCO6A1 |
| | | | | | | | [53,72] |
| 2 | 291 | EKIIYVYMKRKYEAMTKLGF | SSX-S [44,63] | 13 | 1684 | AAASGLSPLQLQKGHVSHPQ | SOX-6 [315,334] |
| 2 | 292 | QPFLKEHRISTEKNWPFLEG | Survivin [11,30] | 13 | 1685 | LKNNLIEKDPSLVYQHLLYL | SPAG1 |
| | | | | | | | [857,876] |
| 2 | 293 | EPRVEVLINRINEVQQAKKK | SYCE1 [50,69] | 13 | 1686 | GQGENKMKNLPVPVYLRPLD | SPAG9 |
| | | | | | | | [660,679] |
| 2 | 294 | NLTLKNHFQSVLEQLELQEK | TAF7L [424,443] | 13 | 1687 | MLKVSRRSLHILSTSKGLIA | SPO11 |
| | | | | | | | [160,179] |
| 2 | 295 | AEDTRLFAPSIKAREPAETY | TAG-1 [231,250] | 13 | 1688 | GQDTSSQKIEDLMEMVQKLQ | SYCE1 [25,44] |
| 2 | 296 | NLEPLVSRDPPASASLFQDT | TAG-2a [64,83] | 13 | 1689 | VWKHGITPPLKNVRKKRFRK | TAF7L [217,236] |
| 2 | 297 | WAERIMESDLRSLQLKKTME | TDRD1 [246,265] | 13 | 1690 | NPVGTVVSREAILREGFLQE | TAG-1 |
| | | | | | | | [115,134] |
| 2 | 298 | TLQEIFQAENTIMLLERSIM | TEKT5 [363,382] | 13 | 1691 | KVLLDAGFAVGEQSMVTDKP | TDRD1 |
| | | | | | | | [665,684] |
| 2 | 299 | NDKDSLSQFWEFSETTASTV | TEX101 [115,134] | 13 | 1692 | QEQMRKLAQRIDIQMRDNRD | TEKT5 [225,244] |
| 2 | 300 | KSDIYSFSMIMQEILTDDIP | TEX14 [436,455] | 13 | 1693 | QAKATQFNSALFTLSSHRQG | TEX14 |
| | | | | | | | [875,894] |
| 2 | 301 | TLVELQMMMETIQFIENKKR | TEX15 [1689,1708] | 13 | 1694 | PQAKEMFIDTVISSYNIETA | TEX15 |
| | | | | | | | [389,408] |
| 2 | 302 | VTKKVVASPRIISLAKPKVR | THEG [326,345] | 13 | 1695 | NEGYDRRPLASMSLPPPKAS | THEG [349,368] |
| 2 | 303 | RRTPTTSSERTINLLEVLPW | TMEM31 [57,76] | 13 | 1696 | RQQYFSDLFNILDTAIIVIL | TPTE [150,169] |
| 2 | 304 | SKIKKIVHSIVSSFAFGLFG | TPTE [79,98] | 13 | 1697 | KSPKSTTAHAILRRVETERD | TSGA10 |
| | | | | | | | [87,106] |
| 2 | 305 | MWMGLIQLVEGVKRKDQGFL | TRAG-3 [1,20] | 13 | 1698 | LSKADGMWPQFRTIQEKEVI | TSP50 [247,266] |
| 2 | 306 | RQNYSSNAYHMSSTMKPNTK | TSGA10 [650,669] | 13 | 1699 | ASAKEGTAFRMEAVQEGAAG | TSPY1 [32,51] |
| 2 | 307 | SSRPRLLWQTPTTQTLPSTT | TSR50 [67,86] | 13 | 1700 | FVDSIFEQWLRRHRPLQEVY | TYR [392,411] |
| 2 | 308 | DEDEDMLSYMVSLEVGEEKH | TSRY1 [175,194] | 13 | 1701 | PPPHSFIKQEPSWGGAEPHE | WT1 [66,85] |
| 2 | 309 | HNALHIYMNGTMSQVQGSAN | TYR [363,382] | 13 | 1702 | SQSSNLSQHQRIHMRENLLM | ZNF165 |
| | | | | | | | [466,485] |
| 2 | 310 | VAKKGKAVRRGRRGKKGAAT | VCX [38,57] | 14 | 1703 | THLESLHLEDNALKVLHNGT | 5T4 [258,277] |
| 2 | 311 | PSQASSGQARMETNAPYLPS | WT1 [117,136] | 14 | 1704 | SPISPHFTVTERQTFQPWPE | ACRBP |
| | | | | | | | [151,170] |
| 2 | 312 | KKKNQQLKVGILHLGSRQKK | XAGE-1 [5,24]; | 14 | 1705 | GETKRLFRELMGDHVSSTKA | ACTL8 |
| | | | XAGE-1b [5,24]; | | | | [306,325] |
| | | | XAGE-1c [84,103]; | | | | |
| | | | XAGE-1d [5,24] | | | | |
| 2 | 313 | PRRSLQPPELIGAMLEFTDE | XAGE-2 [13,32] | 14 | 1706 | DFAKYIEMHVIVEKQLYNHM | ADAM2 |
| | | | | | | | [175,194] |
| 2 | 314 | GRAFNLNSHLIRHQRIHTRE | ZNF165 [406,425] | 14 | 1707 | EDLYVIVNIVDSILDVIGVK | ADAM29 |
| | | | | | | | [223,242] |
| 3 | 315 | DGRLRLARLALVLLGWVSSS | 5T4 [13,32] | 14 | 1708 | KAPQLTSSELMAITRKMAAT | AFP [438,457] |
| 3 | 316 | RNQNPGSLLQLPHTEALLVL | ACRBP [309,328] | 14 | 1709 | ERQLNEAVGNVTPLQLLDWL | AKAP-3 |
| | | | | | | | [830,849] |
| 3 | 317 | DRKKMLEILFELLHVPSVLL | ACTL8 [110,129] | 14 | 1710 | QWKQNATDIMEAMLKRLVSA | AKAP-4 |
| | | | | | | | [414,433] |
| 3 | 318 | RRYIENIYHSKPMRWPFELE | ADAM2 [673,692] | 14 | 1711 | KIRSEFKRKYGKSLYYYIQQ | ANXA2 |
| | | | | | | | [302,321] |
| 3 | 319 | EIKPLAFSTTFEHLVYKMDS | ADAM29 [135,154] | 14 | 1712 | KNGRLTNQLQYLQKVVLKDL | BRDT [24,43] |
| 3 | 320 | QKLGEYYLQNAFLVAYTKKA | AFP [420,439] | 14 | 1713 | LSGEAAEAVHSGTSVKSSSG | CABYR |
| | | | | | | | [451,470] |
| 3 | 321 | SVVSEMMVSIMKTLKIQVKD | AKAR-3 [291,310] | 14 | 1714 | SDTMNVSNLSQGVMLSHSPI | CAGE1 [33,52] |
| 3 | 322 | LDSQKMDMSNIVLMLIQKLL | AKAP-4 [619,638] | 14 | 1715 | GETIKKVHVILHEKNKYHEN | CALR3 |
| | | | | | | | [138,157] |
| 3 | 323 | RDKVLIRIMVSRSEVDMLKI | ANXA2 [284,303] | 14 | 1716 | SDLQFLNENVENSQELIQMY | CCDC62 |
| | | | | | | | [294,313] |
| 3 | 324 | QARLMKEESPVVSWRLEPED | BAGE-1 [16,35]; | 14 | 1717 | TYMEFLWQYKSSADKMQQLN | CDCA1 |
| | | | BAGE-2 [16,35]; | | | | [137,156] |
| | | | BAGE-3 [16,35]; | | | | |
| | | | BAGE-5 [16,35] | | | | |
| 3 | 325 | FVQNTLTKLLKDRRKMQTVQ | BAGE-2 [82,101]; | 14 | 1718 | TILSPKQIKTFFQKILREKD | CT46 [239,258] |
| | | | BAGE-3 [82,101] | | | | |
| 3 | 326 | LKDLWKHSFSWPFQRPVDAV | BRDT [40,59] | 14 | 1719 | QQKLKVMTELYQENEMKLYR | CTAGE2 |
| | | | | | | | [349,368] |
| 3 | 327 | QADIEVMSTVHISSVYNDVP | CARYR [282,301] | 14 | 1720 | SEQDELMADISKRIQSLEDE | CTAGE5 |
| | | | | | | | [160,179] |
| 3 | 328 | SRLEKLLTQVRNLQFMSENE | CAGE1 [507,526] | 14 | 1721 | MSLLSIQQQEGVQVVVQQPG | CTCFL [97,116] |
| 3 | 329 | HLYTLILRPDLSYDVKIDGQ | CALR3 [170,189] | 14 | 1722 | GPLKNTSDVINAAKKIAEAG | CTNNA2 |
| | | | | | | | [733,752] |
| 3 | 330 | NSPTSLLIYKDAFAFNEKAS | CCDC62 [600,619] | 14 | 1723 | HAIELNPSRTLLATGGENPN | DCAF12 |
| | | | | | | | [144,163] |
| 3 | 331 | RETYMEFLWQYKSSADKMQQ | CDCA1 [135,154] | 14 | 1724 | TLSSHLQIDKMTENKTGEVL | DKKL1 [99,118] |
| 3 | 332 | GENLYMSSEPTSWSSAIQSW | CRISP2 [95,114] | 14 | 1725 | VKNSLRGLPGPYVFGQTGNQ | DMRT1 |
| | | | | | | | [253,272] |
| 3 | 333 | GQKYEKIFEMLEGVQGPTAV | CT45 [138,157] | 14 | 1726 | DVDIADVAYYFEKDVKGESL | EpCAM |
| | | | | | | | [206,225] |
| 3 | 334 | YQFKFKYTNNGPLMDFISKN | CT46 [115,134] | 14 | 1727 | ERSVGPLTRKGFYLAFQDIG | EPHA2 |
| | | | | | | | [167,186] |
| 3 | 335 | LGEEEGEQAAGLAAVPRGGS | CT47 [61,80] | 14 | 1728 | KEKLSPDIMKDAYVQKLVKV | FAM46D |
| | | | | | | | [118,137] |
| 3 | 336 | QNFYLPQNYIDQFLLTSFPT | CTAGE1 [27,46] | 14 | 1729 | FNVLEMEVMRRQLYAVNRRL | FATE1 |
| | | | | | | | [129,148] |
| 3 | 337 | LYVRREKKFAVALSGLIEEK | CTAGE2 [40,59] | 14 | 1730 | KFMNPYNAVLTKKFQVTMRG | FBXO39 |
| | | | | | | | [95,114] |
| 3 | 338 | TERLLKMKDWAAMLGEDITD | CTAGE5 [268,287] | 14 | 1731 | DIEDVTPVFPQLSRSIISKL | FSIP1 [433,452] |
| 3 | 339 | SEAVELQDMSLLSIQQQEGV | CTCFL [89,108] | 14 | 1732 | LREEVSTWNSRILKRTAITI | GASZ [439,458] |
| 3 | 340 | SVKRGTMVRAARALLSAVTR | CTNNA2 [117,136] | 14 | 1733 | GSHHTASPWNLSPFSKTSIH | GATA-3 |
| | | | | | | | [104,123] |
| 3 | 341 | SNSIYFSIAIVSEDFVPYKG | CXorf48 [129,148] | 14 | 1734 | KVKNQLRFLAELAYDLDVDD | Glypican-3 |
| | | | | | | | [515,534] |
| 3 | 342 | IVFWKYRRFQRNTGEMSSNS | CXorf61 [16,35] | 14 | 1735 | PDASSIMSTESITGSLGPLL | gp100 |
| | | | | | | | [439,458] |
| 3 | 343 | DTKEDVFVHQTAIKRNNPRK | DBPC [114,133] | 14 | 1736 | HBVTHVYNFDFPRNIEEYVH | HAGE [554,573] |
| 3 | 344 | VKRSLVYYLKNREVRLQNET | DCAF12 [42,61] | 14 | 1737 | NLLLNYGLYRKMEIYRPHKA | HDAC1 [40,59] |
| 3 | 345 | GLTGLQSLLQGFSRLFLKGN | DKKL1 [44,63] | 14 | 1738 | LNYGLYRKMEIYRPHKATAE | HDAC2 [44,63] |
| 3 | 346 | MENRHAMSSQYRMHSYYPPP | DMRT1 [278,297] | 14 | 1739 | FEYFAPDFTLHPDVSTRIEN | HDAC3 |
| | | | | | | | [329,348] |
| 3 | 347 | VDDEESVILTLVPVKDDANM | DPPA2 [18,37] | 14 | 1740 | PATNVSIFINNTQLSDTGTY | IGFS11 [99,118] |
| 3 | 348 | VVAGIVVLVISRKKRMAKYE | EpCAM [279,298] | 14 | 1741 | QDLLDVSFEETVELPQLAEM | JARID1B |
| | | | | | | | [891,910] |
| 3 | 349 | GIMGQFSHHNIIRLEGVISK | EPHA2 [665,684] | 14 | 1742 | RVPSFAAGRVIGKGGKTVNE | KOC1 [493,512] |
| 3 | 350 | NRVAYMNPIAMARWRGPTQS | FAM133A [6,25] | 14 | 1743 | SSKREGAIANAATVLTNMAM | KU-CT-1 |
| | | | | | | | [408,427] |
| 3 | 351 | NGSVASYILASHNGISYKDL | FAM46D [64,83] | 14 | 1744 | RRVHPVSTMVKGLYGIKEEL | LDHC [268,287] |
| 3 | 352 | SAKRVWNMTATRPKKMGSQL | FATE1 [63,82] | 14 | 1745 | SVKDSFRDLFIPRIETILMM | LIPI [49,68] |
| 3 | 353 | SPQFKKTMSTFHNLVSLNLN | FBXO39 [213,232] | 14 | 1746 | DINGFLLLKYRAREPVTKAE | MAGE-A1 |
| | | | | | | | [108,127] |
| 3 | 354 | RESLKMRVSKPFGMLMLSIW | FMR1NB [58,77] | 14 | 1747 | LYDGMEHLIYGEPRKLLTQD | MAGE-A10 |
| | | | | | | | [255,274] |
| 3 | 355 | RPGSRSSNASLEVLSTEPGS | FSIP1 [21,40] | 14 | 1748 | QAQEEDLGINGAQALQACEQ | MAGE-A11 |
| | | | | | | | [127,146] |
| 3 | 356 | DDVALENFFRYFLRLSDDKM | FTHL17 [45,64] | 14 | 1749 | ELVHFLLLKYRAREPFTKAE | MAGE-A12 |
| | | | | | | | [115,134] |
| 3 | 357 | RGRSTYYWPRPRRYVQPPEM | GAGE-7 [4,23] | 14 | 1750 | QSPQGASSLPTTMNYPLWSQ | MAGE-A3 |
| | | | | | | | [63,82] |
| 3 | 358 | DSGISVDSNFQYGWTPLMYA | GASZ [67,86] | 14 | 1751 | AKELVTKAEMLERVIKNYKR | MAGE-A4 |
| | | | | | | | [127,146] |
| 3 | 359 | PGLSHSYMDAAQYPLPEEVD | GATA-3 [29,48] | 14 | 1752 | DPPQSPQGASSLPTTMNYPL | MAGE-A6 |
| | | | | | | | [60,79] |
| 3 | 360 | EQLLQSASMELKFLIIQNAA | Glypican-3 [90,109] | 14 | 1753 | QENYLEYRQAPGSDPVRYEF | MAGE-A8 |
| | | | | | | | [255,274] |
| 3 | 361 | TLIGANASFSIALNFPGSQK | gp100 [76,95] | 14 | 1754 | VIFGKASEFMQVIFGTDVKE | MAGE-A9 |
| | | | | | | | [148,167] |
| 3 | 362 | DRQTVMTSATWPHSVHRLAQ | HAGE [420,439] | 14 | 1755 | VVDEKYKDHFTEILNGASRR | MAGE-B1 |
| | | | | | | | [137,156] |
| 3 | 363 | EEAFYTTDRVMTVSFHKYGE | HDAC1 [184,203]; | 14 | 1756 | KSGSLVQFLLYKYKIKKSVT | MAGE-B2 |
| | | | HDAC2 [185,204] | | | | [114,133] |
| 3 | 364 | SEYFEYFAPDFTLHPDVSTR | HDAC3 [326,345] | 14 | 1757 | NSNPARYEFLWGPRAHAETS | MAGE-B3 |
| | | | | | | | [265,284] |
| 3 | 365 | ATQRDLIATQRDLIVTQRDL | HOM-TES-85 | 14 | 1758 | ACTSKMKVLEFLAKVNETTP | MAGE-B4 |
| | | | [261,280] | | | | [278,297] |
| 3 | 366 | NQPEQVILYQGGQMFDGAPR | IGFS11 [68,87] | 14 | 1759 | EGILSGDNALPKSGLLMSLL | MAGE-B6 |
| | | | | | | | [273,292] |
| 3 | 367 | EGEDLSKKTLLRFWLPFGFI | IL13RA2 [332,351] | 14 | 1760 | SDEQGMSQNRLLILILSIIF | MAGE-C1 |
| | | | | | | | [989,1008] |
| 3 | 368 | TLDDLYPMMNALKLRAESYN | JARID1B [737,756] | 14 | 1761 | ASSTLYLVFSPSSFSTSSSL | MAGE-C2 |
| | | | | | | | [42,61] |
| 3 | 369 | QHIKQLSRFAGASIKIAPAE | KOC1 [426,445] | 14 | 1762 | KQEFLNVQEYNHLLKVMGQY | MORC1 |
| | | | | | | | [414,433] |
| 3 | 370 | DPDFSMYVYEVTKSILIITN | KU-CT-1 [720,739] | 14 | 1763 | EQEKEEIASKSALLRQIKEV | MPHOSPH1 |
| | | | | | | | [207,226] |
| 3 | 371 | HITMPFSSPMEAELVRRILS | Lage-1 [91,110] | 14 | 1764 | GSGSSTTQGQDVTLAPATEP | MUC-1 [67,86] |
| 3 | 372 | TSGKEYSVSANSRIVIVTAG | LDHC [78,97] | 14 | 1765 | LLQEANFHIIDNVELVVSAY | NLRP4 |
| | | | | | | | [572,591] |
| 3 | 373 | PSKGRRWAARAPSTRITYGT | LEMD1 [111,130] | 14 | 1766 | SPGSTLSSSRSVELNGFMAF | NR6A1 |
| | | | | | | | [191,210] |
| 3 | 374 | PEKTMMEGSFSFKLLNQLGM | LIPI [367,386] | 14 | 1767 | NNKLYQLEGLSDITEKAPKV | NXF2 [279,298] |
| 3 | 375 | RALAETSYVKVLEYVIKVSA | MAGE-A1 [269,288] | 14 | 1768 | IPESTYQKVMEINREVEEPP | NY-BR-1 |
| | | | | | | | [442,461] |
| 3 | 376 | DEKVITLVQFLLFKYQMKEP | MAGE-A10 | 14 | 1769 | GRGILIFSDFKDFIFDDMYI | NYD-TSPG |
| | | | [135,154] | | | | [219,238] |
| 3 | 377 | DKIIDLVHLLLRKYRVKGLI | MAGE-A11 | 14 | 1770 | KSQVMKTRLEADEVAAQLER | ODF2 [547,566] |
| | | | [224,243] | | | | |
| 3 | 378 | PDLETSFQVALSNMMAELVH | MAGE-A12 [99,118] | 14 | 1771 | QPDQMRSAEQTRLMPAEQRD | PASD1 |
| | | | | | | | [749,768] |
| 3 | 379 | PQGASSFSTTINYTLWRQSD | MAGE-A2 [65,84] | 14 | 1772 | KRSPRGLSHSPWAVKKINPI | PBK [50,69] |
| 3 | 380 | LSRKVAELVHFLLLKYRARE | MAGE-A3 [109,128] | 14 | 1773 | GSILLPSFQIALLTSEDHIN | PEPP2 |
| | | | | | | | [212,231] |
| 3 | 381 | TTEEQEAAVSSSSPLVPGTL | MAGE-A4 [32,51] | 14 | 1774 | GSSGIIVRLSTNHFRLTSRP | PIWIL1 |
| | | | | | | | [106,125] |
| 3 | 382 | TTEEQEAVSSSSPLVPGTLG | MAGE-A5 [32,51] | 14 | 1775 | KEITFLEYYSKNYGITVKEE | PIWIL2 |
| | | | | | | | [448,467] |
| 3 | 383 | QAALSRKVAKLVHFLLLKYR | MAGE-A6 [106,125] | 14 | 1776 | GTSLEIWLGYVTSVLQYENS | PIWIL3 |
| | | | | | | | [258,277] |
| 3 | 384 | EALDEKVAELVRFLLRKYQI | MAGE-A8 [110,129] | 14 | 1777 | VDSEATRNEWYDFYLISQVA | PIWIL4 |
| | | | | | | | [756,775] |
| 3 | 385 | VIKNYKRYFPVTFGKASEFM | MAGE-A9 [138,157] | 14 | 1778 | DSLFFLRGRLDQLLRHVMNP | PRAME |
| | | | | | | | [304,323] |
| 3 | 386 | NSDPPRYQFLWGFRAYAETT | MAGE-B1 [262,281] | 14 | 1779 | ENIKKFLYNFTQIPHLAGTE | PSMA [68,87] |
| 3 | 387 | FPEILKKASEGLSVVFGLEL | MAGE-B2 [149,168] | 14 | 1780 | IRGRRYPLSEKKIMHYMYQL | RAGE-1 |
| | | | | | | | [93,112] |
| 3 | 388 | YDGKKHFIFGEPRKLITQDL | MAGE-B3 [233,252] | 14 | 1781 | GSMKVKRQFVEFTIKEAARF | SAGE1 |
| | | | | | | | [854,873] |
| 3 | 389 | NSDPPRYQFLWGPRAHAETS | MAGE-B4 [262,281] | 14 | 1782 | PYEEALLQAEAPRLAEYQAY | SART3 |
| | | | | | | | [297,316] |
| 3 | 390 | GLYPHLYEDALIDEVERALR | MAGE-B6 [385,404] | 14 | 1783 | EETRQVYMDLNNNIEKMITA | SCP-1 [205,224] |
| 3 | 391 | EVDPEDSYVFVNTLELTSEG | MAGE-C1 [967,986] | 14 | 1784 | IEKRRKKRSSAGVVEDMGGE | SCP3a [50,69] |
| 3 | 392 | GREHFVYGEPRELLTKVWVQ | MAGE-C2 [261,280] | 14 | 1785 | SQLSLTTKMDAEHPELRSYA | SCRN1 |
| | | | | | | | [189,208] |
| 3 | 393 | EDFPARWSFRAYTSVLYFNP | MORC1 [229,248] | 14 | 1786 | QDKRIENLREKVNILEAQNK | se57-1 |
| | | | | | | | [274,293] |
| 3 | 394 | ADIKKQAEIAHLYIASLFDP | MPHOSPH1 | 14 | 1787 | TTIAFFIYKRRLNENTDFPD | SLCO6A1 |
| | | | [687,706] | | | | [684,703] |
| 3 | 395 | STEKNAVSMTSSVLSSHSPG | MUC-1 [48,67] | 14 | 1788 | QQQEQIARQQQQLLQQQHKI | SOX-6 [232,251] |
| 3 | 396 | IWFQNRRYKTKRKQLSSELG | NKX3.1 [170,189] | 14 | 1789 | LADGNVKAFYRRALAHKGLK | SPAG1 |
| | | | | | | | [686,705] |
| 3 | 397 | QPERLLFVIDSFEELQGGLN | NLRP4 [222,241] | 14 | 1790 | EKKRSSIWQFFSRLFSSSSN | SPAG9 |
| | | | | | | | [546,565] |
| 3 | 398 | MKAINFLNQDIRGLTSASQL | NR6A1 [392,411] | 14 | 1791 | SLILKILSMIYKLVQSNTYA | SPO11 |
| | | | | | | | [112,131] |
| 3 | 399 | ASPQETQSAFSIPVSTLSSS | NXF2 [544,563] | 14 | 1792 | PDVENEVKRLLRSDAEAVST | TAF7L [259,278] |
| 3 | 400 | HHIHEQIMEYIRKLSKNHQN | NY-BR-1 [195,214] | 14 | 1793 | NESVRPYTPFEVKIRSYNRR | TAG-1 |
| | | | | | | | [775,794] |
| 3 | 401 | SRHTPHKTLMPYASLEQSHS | NYD-TSPG [513,532] | 14 | 1794 | LMELNGSSSQLIIMLLKNFM | TDRD1 |
| | | | | | | | [1060,1079] |
| 3 | 402 | EAELARRSLAQBAPPLPVPG | NY-ESO-1 [101,120] | 14 | 1795 | DIPQLKLVNEVFTIDDTLQT | TEKT5 [409,428] |
| 3 | 403 | STNRSMQNYVQFLKSSYANV | ODF2 [788,807] | 14 | 1796 | AEQEHSSKLRHPYLLQLMAV | TEX14 |
| | | | | | | | [294,313] |
| 3 | 404 | SAPSHSISARTKAFRVESTP | ODF3 [118,137] | 14 | 1797 | LRSLLWYDETLYAELLGKPR | TEX15 |
| | | | | | | | [1716,1735] |
| 3 | 405 | RWPVDVSNRIHTSAHVMSMG | ODF4 [115,134] | 14 | 1798 | HVSEHNRLLHLARPKAQSDK | THEG [293,312] |
| 3 | 406 | SRVTNNVVLEAPFLVGIEGS | OIP5 [107,126] | 14 | 1799 | TTEKILIDVFDGLPLYDDVK | TPTE [458,477] |
| 3 | 407 | QAVPAFQGPDMEAFQQELAL | PAGE2 [58,77] | 14 | 1800 | TVEKEMKSLARKAMDTESEL | TSGA10 |
| | | | | | | | [174,193] |
| 3 | 408 | EPAAAAAAAAISDDQIDIAE | PASD1 [227,246] | 14 | 1801 | QMTQTASEVPVLQVIMHSRY | TSP50 [173,192] |
| 3 | 409 | EDPKDRPSAAHIVEALETDV | PBK [303,322] | 14 | 1802 | TPIEWYPDYEVEAYRRRHHN | TSPY1 [233,252] |
| 3 | 410 | PTPESSTIASYVTLRKTKKM | PEPP2 [856,875] | 14 | 1803 | SQVQGSANDPIFLLHHAFVD | TYR [375,394] |
| 3 | 411 | QWALYQYHIDYNPLMEARRL | PIWIL1 [126,145] | 14 | 1804 | LESQPAIRNQGYSTVTEDGT | WT1 [138,157] |
| 3 | 412 | FYNVVERRVMKLLEMKLVGR | PIWIL2 [322,341] | 14 | 1805 | LKPEIHTKEQILELLVLEQF | ZNF165 [78,97] |
| 3 | 413 | HYAHKLAYLVGQSIHQEPNR | PIWIL3 [854,873] | 15 | 1806 | ASNHFLYLPREVLAQLPSLR | 5T4 [218,237] |
| 3 | 414 | FERKLLFSADVSYKVLRNET | PIWIL4 [253,272] | 15 | 1807 | ALSPGKSEDVVLRWSQEFST | ACRBP |
| | | | | | | | [517,536] |
| 3 | 415 | QEAQPLQPSHELDISEDWSL | PLAC1 [184,203] | 15 | 1808 | QNLGEALDFRERQQSALDES | ACTL8 |
| | | | | | | | [216,235] |
| 3 | 416 | NLRRLLLSHIHASSYISPEK | PRAME [261,280] | 15 | 1809 | NETSLPVQITVPEKIRSIIK | ADAM2 [19,38] |
| 3 | 417 | KNSVKTELMKAPMVIMDWEE | PRSS55 [202,221] | 15 | 1810 | FRIVEIVVVIDNYLYIRYER | ADAM29 |
| | | | | | | | [197,216] |
| 3 | 418 | TPLMYSLVHNLTKELKSPDE | PSMA [467,486] | 15 | 1811 | KWVESIFLIFLLNFTESRTL | AFP [2,21] |
| 3 | 419 | EIQALRRLNPHPNILMLHEV | RAGE-1 [50,69] | 15 | 1812 | LGNGSSVDEVSFYANRLTNL | AKAP-3 |
| | | | | | | | [115,134] |
| 3 | 420 | MTPTIRKTQKIVIKKREPLN | RCAS1 [98,117] | 15 | 1813 | YSVYADQVNIDYLMNRPQNL | AKAP-4 |
| | | | | | | | [156,175] |
| 3 | 421 | NGQAASENVESTVPPAFINM | SAGE1 [589,608] | 15 | 1814 | LEKDIISETSGDFRKLMVAL | ANXA2 |
| | | | | | | | [155,174] |
| 3 | 422 | KNPDFKVERYSTSLEKHKLF | SART3 [785,804] | 15 | 1815 | GEKLGRVVHIIQSREPSLSN | BRDT [527,546] |
| 3 | 423 | ASLEIELSNLKAELLSVKKQ | SCP-1 [747,766] | 15 | 1816 | KVSSIHSEQSDVLMVDVATS | CABYR |
| | | | | | | | [182,201] |
| 3 | 424 | AKRKRLEMYTKASLKTSNQK | SCP3a [87,106] | 15 | 1817 | GAKLEKYHSLNEELDFLVTS | CAGE1 |
| | | | | | | | [688,707] |
| 3 | 425 | RSIFKPFIFVEDVKLVPKTQ | SCRN1 [302,321] | 15 | 1818 | KVHVILHFKNKYHENKKLIR | CALR3 |
| | | | | | | | [143,162] |
| 3 | 426 | MEDNSALYESTSAHIIEETE | se57-1 [16,35] | 15 | 1819 | ARNETLSNTLVELSAQVGQL | CCDC62 |
| | | | | | | | [133,152] |
| 3 | 427 | RVVPBKLRSLALGVSYVILR | SLCO6A1 [605,624] | 15 | 1820 | SQEIFLNLKTALEKYHDGIE | CDCA1 |
| | | | | | | | [419,438] |
| 3 | 428 | GSSLDILSSLNSPALFGDQB | SOX-6 [475,494] | 15 | 1821 | NVGEVSTPFHIFKVKVTTER | CT46 [211,230] |
| 3 | 429 | SSEEDKEKEEVAAVKIQAAF | SP17 [106,125] | 15 | 1822 | ATEELETYRKRAKELKEFEK | CTAGE2 |
| | | | | | | | [394,413] |
| 3 | 430 | NRALELHPFSMKPLLRRAMA | SPAG1 [511,530] | 15 | 1823 | ELYQENEMKLHRKLTVEENY | CTAGE5 |
| | | | | | | | [387,406] |
| 3 | 431 | EDGRVQAFGWSLPQKYKQVT | SPAG9 [639,658] | 15 | 1824 | AFQDSVLEEEVELVLAPSEE | CTCFL [55,74] |
| 3 | 432 | ESSTILVVRYRRNVKRTSPE | SPAN-Xc [46,65] | 15 | 1825 | TVMPRFAEQVEVAIEALSAN | CTNNA2 |
| | | | | | | | [580,599] |
| 3 | 433 | EQVRRSISRLTDVSAQDFSM | SPATA19 [141,160] | 15 | 1826 | HLGTLNKVFASQWLNHRQVV | DCAF12 |
| | | | | | | | [85,104] |
| 3 | 434 | QGIRNLVTDAKEVLIVEKDA | SPO11 [208,227] | 15 | 1827 | KEALVPIQKATDSFHTELHP | DKKL1 |
| | | | | | | | [146,165] |
| 3 | 435 | WKKMKYSEKISYVYMKRNYK | SSX-1 [37,56] | 15 | 1828 | ASGALVGAASGSSAGGSSRG | DMRT1 [34,53] |
| 3 | 436 | EKIFYVYMKRKYEAMTKLGF | SSX-2 [44,63] | 15 | 1829 | GDQPNWLRTNWVYRGEAERI | EPHA2 [75,94] |
| 3 | 437 | EWEKMKVSEKIVYVYMKRKY | SSX-3 [36,55] | 15 | 1830 | ASHNGISYKELDVIFGVELP | FAM46D |
| | | | | | | | [73,92] |
| 3 | 438 | EWEKMKSSEKIVYVYMKLNY | SSX-4 [36,55] | 15 | 1831 | ERLARILLQEIPIRSISLRS | FBXO39 |
| | | | | | | | [296,315] |
| 3 | 439 | EWEKMKASEKIIYVYMKRKY | SSX-5 [36,55] | 15 | 1832 | QGLEMRIKLWEEIKSAKYSE | FSIP1 [142,161] |
| 3 | 440 | TLPPAWQPFLKEHRISTFKN | Survivin [5,24] | 15 | 1833 | YAASVANAELVRVLLDRGAN | GASZ [85,104] |
| 3 | 441 | KNKQRQLRLAFEEQLEDLMG | SYCE1 [148,167] | 15 | 1834 | PLPDSMKLESSHSRGSMTAL | GATA-3 |
| | | | | | | | [189,208] |
| 3 | 442 | QRQKDLIMKVENLTLKNHFQ | TAF7L [413,432] | 15 | 1835 | IQNAAVFQEAFEIVVRHAKN | Glypican-3 |
| | | | | | | | [105,124] |
| 3 | 443 | DESTKSVESKFAQLNLAAED | TAG-1 [214,233] | 15 | 1836 | AFTITDQVPFSVSVSQLRAL | gp100 |
| | | | | | | | [206,225] |
| 3 | 444 | GGAESERGLPASTLSRLSNR | TAG-2a [29,48] | 15 | 1837 | FVNLKNITYLVLDEADKMLD | HAGE [384,403] |
| 3 | 445 | GDFYVQLYSSEVLEYMNQLS | TDRD1 [277,296] | 15 | 1838 | RPENMSEYSKQMQRFNVGED | HDAC1 [80,99]; |
| | | | | | | | HDAC2 [81,100] |
| 3 | 446 | DQMWRQFTETNLAFNARISE | TEKT5 [329,348] | 15 | 1839 | PTATQDQVQGTVTIRNISAL | IGFS11 |
| | | | | | | | [189,208] |
| 3 | 447 | MTVEADPANMFNWTTEEVET | TEX101 [34,53] | 15 | 1840 | PYKYKLRYRYTLDDLYPMMN | JARID1B |
| | | | | | | | [727,746] |
| 3 | 448 | QMAYLGSLPVIGEKEVIQAD | TEX14 [226,245] | 15 | 1841 | DQTPDENDQVVVKITGHFYA | KOC1 [526,545] |
| 3 | 449 | LKKSKYFISTYIDFVPYIAS | TEX15 [2172,2191] | 15 | 1842 | FHPGGLMKLRSREADLYRFI | KU-CT-1 |
| | | | | | | | [853,872] |
| 3 | 450 | TERFLEDTTLTITVPAVSRR | THEG [174,193] | 15 | 1843 | KDYSVSANSRIVIVTAGARQ | LDHC [81,100] |
| 3 | 451 | LYDDVKVQFFYSNLPTYYDN | TPTE [472,491] | 15 | 1844 | NDFVNISSIGLTYFQSSNLQ | LIPI [413,432] |
| 3 | 452 | DQVDWSRLLRDAGLVKMSRK | TRAG-3 [52,71] | 15 | 1845 | GREHSAYGEPRKLLTQDLVQ | MAGE-A1 |
| | | | | | | | [226,245] |
| 3 | 453 | MANERISMQNLEALLVANRD | TSGA10 [557,576] | 15 | 1846 | LLTQDWVQENYLEYRQVPGS | MAGE-A10 |
| | | | | | | | [270,289]; |
| | | | | | | | MAGE-A9 |
| | | | | | | | [244,263] |
| 3 | 454 | DNFYHNFTKIPTLVQIIKSQ | TSP50 [274,293] | 15 | 1847 | VGAQALQAEEQEAAFFSSTL | MAGE-A11 |
| | | | | | | | [136,155] |
| 3 | 455 | LVNITEYRASHSTPIEWYPD | TSPY1 [221,240] | 15 | 1848 | SPQGASTLPTTINYTLWSQS | MAGE-A12 |
| | | | | | | | [64,83] |
| 3 | 456 | ADASQSSMHNALHIYMNGTM | TYR [355,374] | 15 | 1849 | REPVTKAEMLGSVVGNWQYF | MAGE-A3 |
| | | | | | | | [127,146]; |
| | | | | | | | MAGE-A6 |
| | | | | | | | [127,146] |
| 3 | 457 | AVRRGRRGKKGAATKMAAVT | VCX [44,63] | 15 | 1850 | WVQENYLEYRQVPGSNPARY | MAGE-A4 |
| | | | | | | | [251,270] |
| 3 | 458 | TWNQMNLGATLKGVAAGSSS | WT1 [237,256] | 15 | 1851 | BSIFGDPKKLLTQYFVQENY | MAGE-A6 |
| | | | | | | | [236,255] |
| 3 | 459 | QTPGINLDLGSGVKVKIIPK | XAGE-1 [46,65]; | 15 | 1852 | QIKEPVTKAEMLESVIKNYK | MAGE-A8 |
| | | | XAGE-1c [125,144]; | | | | [128,147] |
| | | | XAGE-1b [46,65] | | | | |
| 3 | 460 | QLKVGILHLGSRQKKIRIQL | XAGE-1d [10,29] | 15 | 1853 | DGEEHLIYGEPRKFITQDLV | MAGE-B1 |
| | | | | | | | [231,250] |
| 3 | 461 | TPDQKREDDQGAAEIQVPDL | XAGE-2 [47,66] | 15 | 1854 | VNPNGHTYTFIDKVELTDEE | MAGE-B2 |
| | | | | | | | [171,190] |
| 3 | 462 | KEQILELLVLEQFLTILPGD | ZNF165 [85,104] | 15 | 1855 | KMKKPIMKADMLKIVQKSHK | MAGE-B3 |
| | | | | | | | [127,146] |
| 4 | 463 | NSLVSLTYVSFRNLTHLESL | 5T4 [244,263] | 15 | 1856 | LKIISKKYKEHFPEIFRKVS | MAGE-B4 |
| | | | | | | | [136,155] |
| 4 | 464 | EDVVLRWSQEFSTLTLGQFG | ACRBP [524,543] | 15 | 1857 | GSPDAVVSYSKSDVAANGQD | MAGE-B6 |
| | | | | | | | [71,90] |
| 4 | 465 | KTLEFAGQDLSAYLLKSLFK | ACTL8 [171,190] | 15 | 1858 | LQIPVSRSFSSTLLSIFQSS | MAGE-C1 |
| | | | | | | | [198,217] |
| 4 | 466 | RTISLESLAVILAQLLSLSM | ADAM2 [300,319] | 15 | 1859 | YTLDEKVAELVEFLLLKYEA | MAGE-C2 |
| | | | | | | | [139,158] |
| 4 | 467 | SENITPRMQHDTSHLFTTLG | ADAM29 [274,293] | 15 | 1860 | LYRPRKYLYVTSSFKGAFKD | MORC1 |
| | | | | | | | [268,287] |
| 4 | 468 | NFGTRTFQAITVTKLSQKFT | AFP [229,248] | 15 | 1861 | RKKWLEEKMMLITQAKEAEN | MPHOSPH1 |
| | | | | | | | [1459,1478] |
| 4 | 469 | DLRSVFFNFIRNLLSETIFK | AKAP-3 [589,608] | 15 | 1862 | STLVHNGTSARATTTPASKS | MUC-1 |
| | | | | | | | [970,989] |
| 4 | 470 | SDLQKYALGFQHALSPSTST | AKAP-4 [116,135] | 15 | 1863 | QELLVANFEKARRAHWIFLG | NLRP4 |
| | | | | | | | [481,500] |
| 4 | 471 | QNKPLYFADRLYDSMKGKGT | ANXA2 [264,283] | 15 | 1864 | QDFTEYKYTHQPNRFPDLMM | NR6A1 |
| | | | | | | | [423,442] |
| 4 | 472 | SEILKEMLAKKHFSYAWPFY | BRDT [277,296] | 15 | 1865 | KLESAWELGKVKGLKLEELW | NXF2 [307,326] |
| 4 | 473 | AYFQELTMYRGNTTMDIKEL | CARYR [39,58] | 15 | 1866 | LQRKMNVDVSSTIYNNEVLH | NY-BR-1 |
| | | | | | | | [1175,1194] |
| 4 | 474 | KITKQQVFIDVINKLKENVE | CAGE1 [343,362] | 15 | 1867 | TIYVYQEGLVRFATEKFDLS | NYD-TSPG |
| | | | | | | | [269,288] |
| 4 | 475 | RVALATVYFQEEFLDGEHWR | CALR3 [15,34] | 15 | 1868 | DSLVERLHRQTAEYSAFKLE | ODF2 [488,507] |
| 4 | 476 | HPSNFIIEAPGHMSDVEWMS | CCDC62 [517,536] | 15 | 1869 | LETPQDYIRLWQELSDSLGP | PASD1 |
| | | | | | | | [683,702] |
| 4 | 477 | QEKRGAVYERVTTINQEIQK | CDCA1 [381,400] | 15 | 1870 | EAVEENGVITDKADIFAFGL | PBK [216,235] |
| 4 | 478 | NKHNELRKAVSPPASNMLKM | CRGR2 [42,61] | 15 | 1871 | QTESAGIQRAQIQKELWRIQ | PEPP2 |
| | | | | | | | [747,766] |
| 4 | 479 | AGDSLIAGSAMSKAKKLMTG | CT45 [42,61] | 15 | 1872 | LSTNHFRLTSRPQWALYQYH | PIWIL1 |
| | | | | | | | [114,133] |
| 4 | 480 | VGEVSTPFHIFKVKVTTERE | CT46 [212,231] | 15 | 1873 | KSMRFGMLKDHQAVTGNVTA | PIWIL2 |
| | | | | | | | [254,273] |
| 4 | 481 | ENRQLSRLMVGPHAAARNLW | CT47 [143,162] | 15 | 1874 | LKFDTNFLSVPGRVLKNANI | PIWIL3 |
| | | | | | | | [458,477] |
| 4 | 482 | LTERLLKMKDGVAMLEEDVT | CTAGE2 [237,256] | 15 | 1875 | WGLHFGSQISLTGRIVPSEK | PIWIL4 |
| | | | | | | | [434,453] |
| 4 | 483 | PSSETRAFLSPPTLLEGPLR | CTAGE5 [527,546] | 15 | 1876 | PSVSQLSVLSLSGVMLTDVS | PRAME |
| | | | | | | | [346,365] |
| 4 | 484 | KSDLRVHMRNLHAYSAAELK | CTCFL [440,459] | 15 | 1877 | SVYETYELVEKFYDPMFKYH | PSMA [554,573] |
| 4 | 485 | ISDSFLETNVPLLVLIEAAK | CTNNA2 [388,407] | 15 | 1878 | RLNPHPNILMLHEVVFDRKS | RAGE-1 [56,75] |
| 4 | 486 | RLLRLALAFYGRTADPAERQ | CXorf48 [3,22] | 15 | 1879 | TIKEAARFKKVVLIQQLEKA | SAGE1 |
| | | | | | | | [866,885] |
| 4 | 487 | PHSIARQKRILVNLSMVENK | CXorf61 [71,90] | 15 | 1880 | ELTKVRMARQKMSEIFPLTE | SART3 |
| | | | | | | | [127,146] |
| 4 | 488 | PARSQADKPVLAIQVLGTVK | DBPC [80,99] | 15 | 1881 | EELKGTEQELIGLLQAREKE | SCP-1 [458,477] |
| 4 | 489 | GSEWSVYAVGSQAHVSFLDP | DCAF12 [305,324] | 15 | 1882 | EGVGVDINKALLAKRKRLEM | SCP3a [75,94] |
| 4 | 490 | SRLSPRKTHLLYILRPSRQL | DKKL1 [223,242] | 15 | 1883 | QEESITVQTMMNTLRDKASG | SCRN1 |
| | | | | | | | [245,264] |
| 4 | 491 | SSQDSGLVSLSSSSPISNKS | DMRT1 [328,347] | 15 | 1884 | IPPREKMEDNSALYESTSAH | se57-1 [10,29] |
| 4 | 492 | ERAEETNTVEVITSAPGAML | DPPA2 [167,186] | 15 | 1885 | LKTIEKLALEKSYDISSGLV | SLCO6A1 |
| | | | | | | | [139,158] |
| 4 | 493 | QKEITTRYQLDPKFITSILY | EpCAM [167,186] | 15 | 1886 | TSPTQNLFPASKTSPVNLPN | SOX-6 [441,460] |
| 4 | 494 | PGHQKRIAYSLLGLKDQVNT | EPHA2 [952,971] | 15 | 1887 | PGNVKALLRRATTYKHQNKL | SPAG1 |
| | | | | | | | [273,292] |
| 4 | 495 | RGPTQSVGPTIQDYLNRPRP | FAM133A [20,39] | 15 | 1888 | SIKLKDSILSIVHVKGIVLV | SPAG9 |
| | | | | | | | [996,1015] |
| 4 | 496 | LDPMLDFYSDKNAKLTKESY | FAm46D [180,199] | 15 | 1889 | LASSSEVLASIENIIQDIIT | SPO11 [41,60] |
| 4 | 497 | EEQGATWRHRETLIIAVLVS | FATE1 [152,171] | 15 | 1890 | QGHTSSEYDMLREMFSDSRS | TAF7L [311,330] |
| 4 | 498 | WRNSIRSSFISSLSFFLKKM | FBXO39 [141,160] | 15 | 1891 | LWSKGTEILVNSSRVTVTPD | TAG-1 |
| | | | | | | | [451,470] |
| 4 | 499 | RRSHRAMRVAHLELATYELA | FMR1NB [13,32] | 15 | 1892 | IKIVDILEEEVVTFAVEVEL | TDRD1 |
| | | | | | | | [410,429] |
| 4 | 500 | EEEDTFSSVFHTQIPPEEYE | FSIP1 [191,210] | 15 | 1893 | DESTSTLRPPTILPTLRSAL | TEKT5 [66,85] |
| 4 | 501 | LSMAFYFNRDDVALENFFRY | FTHL17 [36,55] | 15 | 1894 | GEYFYSSTAQENLALETSSP | TEX14 |
| | | | | | | | [1088,1107] |
| 4 | 502 | WPRPRRYVQPPEMIGPMRPE | GAGE-7 [11,30] | 15 | 1895 | LDKQRILTVDSFAASSTVPH | TEX15 |
| | | | | | | | [1354,1373] |
| 4 | 503 | LKERDITLRHLLTMREDEFT | GASZ [295,314] | 15 | 1896 | EDTTLTITVPAVSRRVEELS | THEG [179,198] |
| 4 | 504 | FPKNSSFNPAALSRHMSSLS | GATA-3 [386,405] | 15 | 1897 | AYDPKHFHNRVVRIMIDDHN | TPTE [289,308] |
| 4 | 505 | SMELKFLIIQNAAVFQEAFE | Glypican-3 | 15 | 1898 | SRDVAQFRNVVTQLEADLDI | TSGA10 |
| | | | | | | | [610,629] |
| 4 | 506 | AHSSSAFTITDQVPFSVSVS | gp100 [201,220] | 15 | 1899 | TTQTLPSTTMETQFPVSEGK | TSP50 [78,97] |
| 4 | 507 | EEEKWSHMQTFLQSMSSTDK | HAGE [470,489] | 15 | 1900 | LVERREEAQRAQQAVPGPGP | TSPY1 [83,102] |
| 4 | 508 | EKIKQRLFENLRMLPHAPGV | HDAC1 [360,379]; | 15 | 1901 | KNGSTPMFNDINIYELFVWM | TYR [160,179] |
| | | | HDAC2 [361,380] | | | | |
| 4 | 500 | WTYETSLLVEEAISEELPYS | HDAC3 [307,326] | 15 | 1902 | QRKFSRSDHLKTHTRTHTGK | WT1 [389,408] |
| 4 | 510 | PNHPSRKKVNFLDMSLDDII | HOM-TES-85 [15,34] | 15 | 1903 | ENSRSMPKLEIFEKIESQRI | ZNF165 |
| | | | | | | | [188,207] |
| 4 | 511 | SSDNNTLTSSNAYNSRYWSN | IGFS11 [305,324] | 16 | 1904 | HLPSLRQLDLSHNPLADLSP | 5T4 [140,159] |
| 4 | 512 | GSETWKTIITKNLHYKDGFD | IL13RA2 [77,96] | 16 | 1905 | KEGREAVSQLQTDSEPKFHS | ACRBP |
| | | | | | | | [239,258] |
| 4 | 513 | PYSAVEKAMARLQELLTVSE | JARID1B [950,969] | 16 | 1906 | TLEFAGQDLSAYLLKSLFKE | ACTL8 |
| | | | | | | | [172,191] |
| 4 | 514 | EAQDIKFTEEIPLKILAHNN | KOC1 [267,286] | 16 | 1907 | QLYNHMGSDTTVVAQKVFQL | ADAM2 |
| | | | | | | | [189,208] |
| 4 | 515 | LLRELDVMNSVIAQLAPEEE | KU-CT-1 [103,122] | 16 | 1908 | VFTYTDQGAILEDQPFVQNN | ADAM29 |
| | | | | | | | [80,99] |
| 4 | 516 | AELVRRILSRDAAPLPRPGA | Lage-1 [102,121] | 16 | 1909 | EYSRRHPQLAVSVILRVAKG | AFP [357,376] |
| 4 | 517 | NLDSARFRYLIGEKLGVHPT | LDHC [164,183] | 16 | 1910 | MDTSTDPVRVLSWLRRDLEK | AKAP-3 [32,51] |
| 4 | 518 | LQGNIILSTEKSKKLKKWPE | LEMD1 [74,93] | 16 | 1911 | SKIASEMAYEAVELTAAEMR | AKAP-4 |
| | | | | | | | [268,287] |
| 4 | 519 | GSIPLWLQNFVRILLNEEDM | LIPI [108,127] | 16 | 1912 | ETVILGLLKTPAQYDASELK | ANXA2 [96,115] |
| 4 | 520 | FPEIFGKASESLQLVEGIDV | MAGE-A1 [140,159] | 16 | 1913 | DKSKLWLLKDRDLARQKEQE | BRDT [900,919] |
| 4 | 521 | DGMEHLIYGEPRKLLTQDWV | MAGE-A10 | 16 | 1914 | PGTEQTEAVGGLSSKPATPK | CABYR |
| | | | [257,276] | | | | [129,148] |
| 4 | 522 | EEQEAAFFSSTLNVGTLEEL | MAGE-A11 | 16 | 1915 | PVQEDMALNEVLQKLKHTNR | CAGE1 |
| | | | [144,163] | | | | [298,317] |
| 4 | 523 | QDFFPVIFSKASEYLQLVFG | MAGE-A12 | 16 | 1916 | HKEKDKGLQTTQNGREYAIS | CALR3 [59,78] |
| | | | [144,163]; MAGE-A2 | | | | |
| | | | [144,163] | | | | |
| 4 | 524 | PDLESEFQAALSNKVAELVH | MAGE-A3 [99,118] | 16 | 1917 | PTLSDEKQWHDVSVYLGLTN | CCDC62 |
| | | | | | | | [453,472] |
| 4 | 525 | NKVDELAHELLRKYRAKELV | MAGE-A4 [112,131] | 16 | 1918 | DLSDNREKLASILKESLNLE | CDCA1 |
| | | | | | | | [302,321] |
| 4 | 526 | AIPTAIDFTLWRQSIKGSSN | MAGE-A5 [70,89] | 16 | 1919 | ESSMLSTDTKKASILLIRKI | CT46 [138,157] |
| 4 | 527 | PDLESEFQAALSRKVAKLVH | MAGE-A6 [99,118] | 16 | 1920 | TEIEFKIKLLEKBPYGLDVP | CTAGE2 |
| | | | | | | | [458,477] |
| 4 | 528 | EEQKAASSSSTLIMGTLEEV | MAGE-A8 [34,53] | 16 | 1921 | IEEKSKLLEKFSLVQKEYEG | CTAGE5 |
| | | | | | | | [86,105] |
| 4 | 529 | QGGASSSISVYYTLWSQFDE | MAGE-A9 [62,81] | 16 | 1922 | TSGELVEHRRYKHTHEKPFK | CTCFL [324,343] |
| 4 | 530 | ETTATTFRARSNAPFSESSH | MAGE-B1 [326,345] | 16 | 1923 | KKTRDLREQLRKAVMDHISD | CTNNA2 |
| | | | | | | | [371,390] |
| 4 | 531 | PLTRKSGSLVQFLLYKYKIK | MAGE-B2 [110,129] | 16 | 1924 | PSLLKEREFHLGTLNKVFAS | DCAF12 [76,95] |
| 4 | 532 | EERVQAAAMLNDGSSAMGRK | MAGE-B3 [315,334] | 16 | 1925 | EHQLGNNTLSSHLQIDKMTD | DKKL1 [92,111] |
| 4 | 533 | RVAANNGTTAMTSAYSRATS | MAGE-B4 [320,339] | 16 | 1926 | GSSEGGGSGSGASDLGAGSK | DMRT1 [49,68] |
| 4 | 534 | VEREYKPYFPQILNETSQHL | MAGE-B6 [225,244] | 16 | 1927 | LEGRSTTSLSVSWSIFETQQ | EPHA2 |
| | | | | | | | [444,463] |
| 4 | 535 | ISRYTGYFPVIFRKAREFIE | MAGE-C1 [939,958] | 16 | 1928 | PTMEVKPKDIIHVVKDQLIG | FAM46D |
| | | | | | | | [34,53] |
| 4 | 536 | VESPSSFSTSSSLILGGPEE | MAGE-C2 [49,68] | 16 | 1929 | FPSRVHASEVESAVWYVKKF | FBXO39 [66,85] |
| 4 | 537 | TSVLYFNPWMRIFIQAKRVK | MORC1 [241,260] | 16 | 1930 | FIKRNIELAKESENFVVMVD | FSIP1 [254,273] |
| 4 | 538 | KFSVWVSFFEIYNEYIYDLF | MPHOSPH1 | 16 | 1931 | TQVVALLVAHGAEVNTQDEN | GASZ [162,181] |
| | | | [268,287] | | | | |
| 4 | 539 | NGGSSLSYTNPAVAAASANL | MUC-1 [1236,1255] | 16 | 1932 | KDNEISTEHNLGNVHSFLKL | Glypican-3 |
| | | | | | | | [545,564] |
| 4 | 540 | ELERKFSHQKYLSAPERAHL | NKX3.1 [138,157] | 16 | 1933 | DIVQGIESAEILQAVFSGEG | gp100 |
| | | | | | | | [488,507] |
| 4 | 541 | MQDPAFVKQAVNLLQEANFH | NLRP4 [560,579] | 16 | 1934 | GSTDNNVVAGDRPLIDWDQI | HAGE [157,176] |
| 4 | 542 | SSRSVELNGFMAFREQYMGM | NR6A1 [198,217] | 16 | 1935 | GSVAGAVKLNRQQTDMAVNW | HDAC2 |
| | | | | | | | [117,136] |
| 4 | 543 | IFVNHSTAPYSVKNKLKPGQ | NXF2 [194,213] | 16 | 1936 | EGSSPQVMSRSNGSVSRKPR | IGFS11 |
| | | | | | | | [378,397] |
| 4 | 544 | EKNEEIFNYNNHLKNRIYQY | NY-BR-1 | 16 | 1937 | LPEIQELYQTLLAKFSPAQQ | JARID1B |
| | | | [1312,1331] | | | | [1360,1379] |
| 4 | 545 | SNLQNNYAHLTNSSINKSGA | NYD-TSPG [288,307] | 16 | 1938 | SDLESIFKDAKIFVSGETLV | KOC1 [16,35] |
| 4 | 546 | ILTIRLTAADHRQLQLSISS | NY-ESO-1 [132,151] | 16 | 1939 | SPLRSANTVVQSKAALAVTA | KU-CT-1 |
| | | | | | | | [446,465] |
| 4 | 547 | EDSERLMEQQGALLKELAEA | ODF2 [281,300] | 16 | 1940 | QHGSLFFSTSKITSGKDYSV | LDHC [66,85] |
| 4 | 548 | KASQPSFSIKGRSKLGGESD | ODF3 [155,174] | 16 | 1941 | PRLGYQAKLFKGVLKERMEG | LIPI [319,338] |
| 4 | 549 | RITHSFRWMAQVLASELSLV | ODF4 [71,90] | 16 | 1942 | YRQVPDSDPARYEFLWGFRA | MAGE-A1 |
| | | | | | | | [251,270] |
| 4 | 550 | THNRLKSLMKILSEVTPDQS | OIP5 [206,225] | 16 | 1943 | YRQVPGSDPARYEFLWGFRA | MAGE-A10 |
| | | | | | | | [283,302] |
| 4 | 551 | DGPDVREGIMPTFDLTKVLE | PAGE2 [86,105] | 16 | 1944 | YEFLWGPRAHAETSKMKVLE | MAGE-A11 |
| | | | | | | | [382,401] |
| 4 | 552 | EVNPLYRADPVDLEFSVDQV | PASD1 [292,311] | 16 | 1945 | ILGDPKKLLTQHFVQENYLE | MAGE-A3 |
| | | | | | | | [238,257] |
| 4 | 553 | IILKVALNMARGLKYLHQEK | PBK [142,161] | 16 | 1946 | ASESLKMIEGIDVKEVDFAS | MAGE-A4 |
| | | | | | | | [155,174] |
| 4 | 554 | DHINRKYAFKAAHPNMRTYY | PEPP2 [228,247] | 16 | 1947 | SVMGLYDGREHSVYWKLRKL | MAGE-A8 |
| | | | | | | | [229,248] |
| 4 | 555 | VTPAMGMQMRKAIMIEVDDR | PIWIL1 [518,537] | 16 | 1948 | YKMREPIMKADMLKVVDEKY | MAGE-B1 |
| | | | | | | | [123,142] |
| 4 | 556 | ESVGLVSMFRGLGIETVSKT | PIWIL2 [67,86] | 16 | 1949 | DGEEHSVFGEEWKLITKELV | MAGE-B2 |
| | | | | | | | [234,253] |
| 4 | 557 | FGERHIEDGNSLLLSRELKE | PIWIL3 [170,189] | 16 | 1950 | KVESTKESYVLVSKMDLENN | MAGE-B3 |
| | | | | | | | [170,189] |
| 4 | 558 | ADVSYKVLRNETVLEFMTAL | PIWIL4 [261,280] | 16 | 1951 | NETTHSYILVSMLGENDGNQ | MAGE-B4 |
| | | | | | | | [170,189] |
| 4 | 559 | HELDISEDWSLHTDDMIGSM | PLAC1 [193,212] | 16 | 1952 | LDEKVDELARFLLLKYQVKQ | MAGE-C1 |
| | | | | | | | [908,927] |
| 4 | 560 | YLGQMINLRRLLLSHIHASS | PRAME [255,274] | 16 | 1953 | QATIDTADDATVMASESLSV | MAGE-C2 |
| | | | | | | | [345,364] |
| 4 | 561 | ENDIALLLLASPIKLEDLKV | PRSS55 [154,173] | 16 | 1954 | FNQIQNTYMVQYEKKIKRKL | MORC1 |
| | | | | | | | [866,885] |
| 4 | 562 | EMKTYSVSEDSLFSAVKNFT | PSMA [621,640] | 16 | 1955 | RTLDSVSQISNIDLLNLREL | MPHOSPH1 |
| | | | | | | | [993,1012] |
| 4 | 563 | ELMEMNIYELIRGRRYELSE | RAGE-1 [83,102] | 16 | 1956 | HNGTSARATTTEASKSTEFS | MUC-1 |
| | | | | | | | [974,993] |
| 4 | 564 | EEEEDAAWQAEEVLRQQKLA | RCAS1 [160,179] | 16 | 1957 | MAASFFSDEGLMWYLEELKK | NLRP4 [1,20] |
| 4 | 565 | DQEQPNNVLSTVQPVITYLT | SAGE1 [308,327] | 16 | 1958 | LQMGMNRKAIREDGMEGGRN | NR6A1 |
| | | | | | | | [121,140] |
| 4 | 566 | ITYVEIWQAYLDYLRRRVDF | SART3 [415,434] | 16 | 1959 | DGYTRNMEKVTIPYGIKYDK | NXF2 [118,137] |
| 4 | 567 | DKKTQTELLETEETYWKLDS | SCP-1 [793,812] | 16 | 1960 | IDIHFLERKMQHHLLKEKNE | NY-BR-1 |
| | | | | | | | [1296,1315] |
| 4 | 568 | QNEFKKEMAMLQKKIMMETQ | SCP3a [201,220] | 16 | 1961 | RTTTPAFTLNIPSEANHTEQ | NYD-TSPG |
| | | | | | | | [19,38] |
| 4 | 569 | HSFQSAYLIVERDEAWVLET | SCRN1 [151,170] | 16 | 1962 | ITEESSEKLVSVMRLSELST | ODF2 [91,110] |
| 4 | 570 | LQFVIHSQHQNLRSVIQEME | se57-1 [246,266] | 16 | 1963 | SSTVELDTMEESPALSLQDF | PASD1 |
| | | | | | | | [268,287] |
| 4 | 571 | IIFTTIAFFIYKRRLNENTD | SLCO6A1 [681,700] | 16 | 1964 | KERETSMINEASNYNVTSDY | PEPP2 |
| | | | | | | | [115,134] |
| 4 | 572 | IGEYKQLMRSRRQEMRQFFT | SOX-6 [707,726] | 16 | 1965 | ELDNWLLIYTRRNYEAANSL | PIWIL1 |
| | | | | | | | [492,511] |
| 4 | 573 | GSKVEDREYNNHAFEEQEPE | SP17 [61,80] | 16 | 1966 | DRIETYVRTIQSTLGAEGKI | PIWIL2 |
| | | | | | | | [647,666] |
| 4 | 574 | SMKELLRRAMAYETLEQYGK | SPAG1 [520,539] | 16 | 1967 | DGDANSYIDTLRKYTRETLQ | PIWIL3 |
| | | | | | | | [549,568] |
| 4 | 575 | RLDSTLRLYHAHTYQHLQDV | SPAG9 [1107,1126] | 16 | 1968 | GQLKTLIEYEVPQLLSSVAE | PIWIL4 |
| | | | | | | | [698,717] |
| 4 | 576 | TILVVRYRRNVKRTSPEELL | SPAN-Xc [49,68] | 16 | 1969 | TSERRLVELAGQSLLKDEAL | PRAME [21,40] |
| 4 | 577 | RWSHTRIFQVPSEMTEDIMR | SPATA19 [118,137] | 16 | 1970 | PHLAGTEQNFQLAKQIQSQW | PSMA [81,100] |
| 4 | 578 | ASIENIIQDIITSLARNEAP | SPO11 [49,68] | 16 | 1971 | NETFPFKKGSGIELLTTNLS | RAGE-1 |
| | | | | | | | [236,255] |
| 4 | 579 | TEGRLHRIIPKIMEKKEAED | SSX-1 [100,119] | 16 | 1972 | AMSSRDLYATITHSVREEKM | SAGE1 |
| | | | | | | | [474,493] |
| 4 | 580 | AWTHRLRERKQLVIYEEISD | SSX-2 [163,182] | 16 | 1973 | AATEAPKMSNADFAKLFLRK | SART3 |
| | | | | | | | [944,963] |
| 4 | 581 | AMTKLGFKAILESFMRNKRV | SSX-3 [57,76] | 16 | 1974 | EVEKAKVIADEAVKLQKEID | SCP-1 [681,700] |
| 4 | 582 | TEGSLQRIFEKIMEKKPAEE | SSX-4 [100,119] | 16 | 1975 | SGKESVEDQFTRAYDFETED | SCP3a [12,31] |
| 4 | 583 | EAMTKLGFKATLEPFMRNKR | SSX-5 [56,75] | 16 | 1976 | EHPELRSYAQSQGWWTGEGE | SCRN1 |
| | | | | | | | [200,219] |
| 4 | 584 | TNNKKKEFEETAKKVRRATE | Survivin [117,136] | 16 | 1977 | LKIKLQASREAGAAALRNVA | se57-1 |
| | | | | | | | [112,131] |
| 4 | 585 | RKAEELLAAAAQRHQQLQQK | SYCE1 [248,267] | 16 | 1978 | SGSQDEVSRGVEPLEAARAQ | SLCO6A1 [9,28] |
| 4 | 586 | LRTLEKKTFYKTADISQMLV | TAF7L [163,182] | 16 | 1979 | PERRKGSLADVVDTLKQKKL | SOX-6 [120,139] |
| 4 | 687 | WMDYEFRVIASNILGTGEES | TAG-1 [678,697] | 16 | 1980 | DTELLLIQSLKNNLIEKDES | SPAG1 |
| | | | | | | | [848,867] |
| 4 | 588 | YRASVLAYASEESVLVGYVD | TDRD1 [559,578] | 16 | 1981 | NQYKERLMELQEAVRWTEMI | SPAG9 |
| | | | | | | | [516,535] |
| 4 | 589 | GEWKSELSYELDRLLTENQN | TEKT5 [149,168] | 16 | 1982 | VTDAKEVLIVEKDATFQRLL | SPO11 |
| | | | | | | | [214,233] |
| 4 | 590 | QADDEPTESFFSGEYMVMTN | TEX14 [243,262] | 16 | 1983 | DRGSETTYESSADIAGDEGT | TAF7L [36,55] |
| 4 | 591 | YIEIVMVSETIHFLKNSIAK | TEX15 [2062,2081] | 16 | 1984 | GGAEGELIVNWTEMSREYQN | TAG-1 |
| | | | | | | | [721,740] |
| 4 | 592 | MEMSEVSQVSRAAQMAVESS | THEG [247,266] | 16 | 1985 | GEGSWYRALVKEILENGHVK | TDRD1 |
| | | | | | | | [775,794] |
| 4 | 593 | FIKHFIIYSIPRYVRELKIQ | TPTE [415,434] | 16 | 1986 | EEAEHLFETLSDQMWRQFTD | TEKT5 [318,337] |
| 4 | 594 | DAGLVKMSRKPRASSPLSNN | TRAG-3 [62,81] | 16 | 1987 | QAIIQGFSYDLLKKIDSPQR | TEX14 |
| | | | | | | | [152,171] |
| 4 | 595 | LNAERSYKSQISTLHKSVVK | TSGA10 [470,489] | 16 | 1988 | LKKAHRRVHTSLQLITKVGE | TEX15 |
| | | | | | | | [1004,1023] |
| 4 | 596 | IWRDVIYSVRVGSPWIDQMT | TSR50 [156,175] | 16 | 1989 | TPVWPIPRSSLEYRASSRLK | THEG [214,233] |
| 4 | 597 | GEEAVLLLDDIMAEVEVVAE | TSRY1 [60,79] | 16 | 1990 | RRILFIKHFIIYSIPRYVRD | TRTE [411,430] |
| 4 | 598 | HYYVSMDALLGGSEIWRDID | TYR [180,199] | 16 | 1991 | LLYEQAQEEITRLRREMMKS | TSGA10 [66,85] |
| 4 | 599 | GAQYRIHTHGVFRGIQDVRR | WT1 [283,302] | 16 | 1992 | IMHSRYRAQRFWSWVGQAND | TSR50 [187,206] |
| 4 | 600 | TAITREEGGPRSGGAQAKLG | XAGE-1c [13,32] | 16 | 1993 | AVQVELEPVNAQARKAFSRQ | TSPY1 [114,133] |
| 4 | 601 | EGGTDVKGKILPKAEHFKMP | XAGE-2 [83,102] | 16 | 1994 | HPTNPNLLSPASFFSSWQIV | TYR [256,275] |
| 4 | 602 | DLERGTDEAVLQVQAHEHGQ | ZNF165 [126,145] | 16 | 1995 | GHTPSHHAAQFPNHSFKHED | WT1 [161,180] |
| 5 | 603 | EVRAGAFEHLPSLRQLDLSH | 5T4 [132,151] | 16 | 1996 | FAESSDLTRHRRIHTGERPF | ZNF165 |
| | | | | | | | [381,400] |
| 5 | 604 | SPNTLKEIEASAEVSPTTMT | ACRBP [131,150] | 17 | 1997 | HMADMVTWLKETEVVQGKER | 5T4 [301,320] |
| 5 | 605 | DQLQMSLYASGLLTGVVVDS | ACTL8 [131,150] | 17 | 1998 | SQASYKIVIEGKPYTVNLMQ | ADAM2 [43,62] |
| 5 | 606 | SYLVLRPHDVAFLLVYREKS | ADAM2 [256,275] | 17 | 1999 | IWTNKNLIVVDDVRKSVHLY | ADAM29 |
| | | | | | | | [250,269] |
| 5 | 607 | DSEEKQFSTMRSGFMQNEIT | ADAM29 [153,172] | 17 | 2000 | ESRTLHRNEYGIASILDSYQ | AFP [17,36] |
| 5 | 608 | ASFVHEYSRRHPQLAVSVIL | AFP [352,371] | 17 | 2001 | VSDLIDSFLRNLHSVTGTLM | AKAP-3 |
| | | | | | | | [330,349] |
| 5 | 609 | DEFTASVSEGIMTYANSVVS | AKAP-3 [275,294] | 17 | 2002 | SIDDLSFYVNRLSSLVIQMA | AKAP-4 |
| | | | | | | | [213,232] |
| 5 | 610 | QWIAASQFNVPMLYFMGDKD | AKAP-4 [779,798] | 17 | 2003 | MKGKGTRDKVLIRIMVSRSE | ANXA2 |
| | | | | | | | [278,297] |
| 5 | 611 | RKYGKSLYYYIQQDTKGDYQ | ANXA2 [309,328] | 17 | 2004 | SATEKVFKQQEIPSVFPKTS | BRDT [166,185] |
| 5 | 612 | EPEDGTALDVHFVSTLEPLS | RAGE-2 [32,51]; | 17 | 2005 | PADPAQLAAQMLGKVSSIHS | CABYR |
| | | | BAGE-3 [32,51] | | | | [169,188] |
| 5 | 613 | VGTIBMTLQSDIMTMFENNF | BRDT [927,946] | 17 | 2006 | ALIQPVDTISISSLRQFETV | CAGE1 |
| | | | | | | | [105,124] |
| 5 | 614 | DQSDVLMVDVATSMPVVIKE | CARYR [189,208] | 17 | 2007 | IDQKNLNGKSQYYIMFGPDI | CALR3 |
| | | | | | | | [117,136] |
| 5 | 615 | NNIENYSTNALIQPVDTISI | CAGE1 [96,115] | 17 | 2008 | AVHQQQLLSWEEDRQKVLTL | CCDC62 [49,68] |
| 5 | 616 | ESGSIEYDWNLTSLKKETSP | CALR3 [192,211] | 17 | 2009 | LQQSLNQDFHQKTIVLQEGN | CDCA1 |
| | | | | | | | [190,209] |
| 5 | 617 | ELHKRTEIIRSLTKKVKALE | CCDC62 [78,97] | 17 | 2010 | LQESQKQLLQEAEVWKEQVS | CTAGE2 |
| | | | | | | | [188,207] |
| 5 | 618 | EREKLKSQEIFLNLKTALEK | CDCA1 [413,432] | 17 | 2011 | EEPGVTPQPYLGLLLEELRR | CTAGE5 [2,21] |
| 5 | 619 | SPPASNMLKMEWSREVTTNA | CRISR2 [52,71] | 17 | 2012 | VLQFHALEENVMVASEDSKL | CTCFL [150,169] |
| 5 | 620 | TEKTEKVAVDPETVFKRPRE | CT45 [2,21] | 17 | 2013 | RDEMAAARGALKKNATMLYT | CTNNA2 |
| | | | | | | | [203,222] |
| 5 | 621 | PNKISTEHQSLVLVKRLLAV | CT46 [17,36] | 17 | 2014 | QNETSYSRVLHGYAAQQLPS | DCAF12 [58,77] |
| 5 | 622 | NDHILIENRQLSRLMVGPHA | CT47 [137,156] | 17 | 2015 | RLFLKGNLLRGIDSLFSAPM | DXKL1 [57,76] |
| 5 | 623 | SKSLKSQVAEAKMTFKRFQA | CTAGE2 [150,169] | 17 | 2016 | NSYNVRRTEGFSVTLDDLAP | EPHA2 |
| | | | | | | | [480,499] |
| 5 | 624 | VGFFAVLFFLWRSFRSVRSR | CTAGE5 [50,69] | 17 | 2017 | FGVELPGNEEFQVVKDAVLD | FAM46D |
| | | | | | | | [87,106] |
| 5 | 625 | SAVFHERYALIQHQKTHKNE | CTCFL [464,483] | 17 | 2018 | LSKSNNRLKSLSIQYLELDR | FBXO39 |
| | | | | | | | [119,138] |
| 5 | 626 | KNLMNAVVLTVKASYVASTK | CTNNA2 [870,889] | 17 | 2019 | QLAEIDIKLQELSAASPTIS | FSIP1 [319,338] |
| 5 | 627 | IDESIYFSSDVVTGNVPLKV | CXorf48 [53,72] | 17 | 2020 | TKLEISGDEFLNFLLKLNKQ | GASZ [347,366] |
| 5 | 628 | KRILVNLSMVENKLVELEHT | CXorf61 [78,97] | 17 | 2021 | DKYWREYILSLEELVNGMYR | Glypican-3 |
| | | | | | | | [293,312] |
| 5 | 629 | NRRKSRRFIPRPPSVAPPPM | DBPC [176,195] | 17 | 2022 | RNQPLTFALQLHDPSGYLAE | gp100 |
| | | | | | | | [234,253] |
| 5 | 630 | EVRLQNETSYSRVLHGYAAQ | DCAF12 [54,73] | 17 | 2023 | GTLELVAVSSVKQNIIVTTE | HAGE [451,470] |
| 5 | 631 | LQGFSRLFLKGNLLRGIDSL | DKKL1 [52,71] | 17 | 2024 | PHAPGVQMQAIPEDAVHEDS | HDAC2 |
| | | | | | | | [375,394] |
| 5 | 632 | NLIAERQRVMAAQVALRRQQ | DMRT1 [106,125] | 17 | 2025 | SSNAYNSRYWSNNPKVHRNT | IGFS11 |
| | | | | | | | [313,332] |
| 5 | 633 | RKAVTKRARLQRSYEMNERA | pPPA2 [150,169] | 17 | 2026 | AKINKKKSLVSFKALIEESE | JARID1B |
| | | | | | | | [768,787] |
| 5 | 634 | KTQNDVDIADVAYYFEKDVK | EpCAM [202,221] | 17 | 2027 | NDIASMNLQAHLIPGLNLNA | KOC1 [353,372] |
| 5 | 635 | GLVTSRSFRTASVSINQTEP | EPHA2 [420,439] | 17 | 2028 | LLGSENDGTKIAASQAISAM | KU-CT-1 |
| | | | | | | | [324,343] |
| 5 | 636 | KQLENKKTGSKALAEFEEKM | FAM133A [46,65] | 17 | 2029 | ETRLALVQRNVAIMKSIIPA | LDHC [104,123] |
| 5 | 637 | IVKDARLNGSVASYILASHN | FAM46D [57,76] | 17 | 2030 | LQNFVRILLNEEDMNVIVVD | LIPI [114,133] |
| 5 | 638 | EFNVLEMEVMRRQLYAVNRR | FATE1 [128,147] | 17 | 2031 | QGASAFPTTINFTRQRQPSE | MAGE-A1 |
| | | | | | | | [59,78] |
| 5 | 639 | RKLFYFKIWAFLDVSFVERI | FBXO39 [366,385] | 17 | 2032 | ITKAEILESVIRNYEDHFPL | MAGE-A10 |
| | | | | | | | [155,174] |
| 5 | 640 | KAKGRNRRSHRAMRVAHLEL | FMR1NB [7,26] | 17 | 2033 | FGEPKRLLTQNWVQEKYLVY | MAGE-A11 |
| | | | | | | | [352,371] |
| 5 | 641 | PVFPQLSRSIISKLLNESET | FSIP1 [439,458] | 17 | 2034 | ASEEEIWEELGVMGVYDGRE | MAGE-A4 |
| | | | | | | | [217,236] |
| 5 | 642 | KNVNQSLLDLYQLAVEKGDP | FTH117 [109,128] | 17 | 2035 | KLVHFLLLKYRAREPVTKAE | MAGE-A6 |
| | | | | | | | [115,134] |
| 5 | 643 | GWEIGYLERTSQKLKRLLPI | GASZ [24,43] | 17 | 2036 | MLLGQKSQRYKAEEGLQAQG | MAGE-A8 [1,20] |
| 5 | 644 | SSLSHISPFSHSSHMLTTPT | GATA-3 [402,421] | 17 | 2037 | NLSNDWDFPRNGLLMPLLGV | MAGE-B1 |
| | | | | | | | [188,207] |
| 5 | 645 | KHINQLLRTMSMPKGRVLDK | Glypican-3 | 17 | 2038 | SLLSSWDFPRRKLLMPLLGV | MAGE-B2 |
| | | | [467,486] | | | | [191,210] |
| 5 | 646 | LNVSLADTNSLAVVSTQLIM | gp100 [567,586] | 17 | 2039 | VDVSYKKSYKGANSKIEKKQ | MAGE-B3 |
| | | | | | | | [75,94] |
| 5 | 647 | RNRPGMLVLTPTRELALQVE | HAGE [314,333] | 17 | 2040 | ARPRVAARRGTTAMTSAYSR | MAGE-B4 |
| | | | | | | | [317,336] |
| 5 | 648 | FKPVMSKVMEMFQPSAVVLQ | HDAC1 [241,260] | 17 | 2041 | PQSPLQGEEFQSSLQSPVSI | MAGE-C1 |
| | | | | | | | [612,631] |
| 5 | 649 | RQQTDMAVNWAGGLHHAKKS | HDAC2 [127,146] | 17 | 2042 | ASEEVIWEVLNAVGVYAGRE | MAGE-C2 |
| | | | | | | | [244,263] |
| 5 | 650 | QSYKHLFQPVINQVVDFYQP | HDAC3 [230,249] | 17 | 2043 | HESLSSFELSASRRGQKRNI | MORC1 |
| | | | | | | | [606,625] |
| 5 | 651 | SPSRQQSKAHRHRHRRGYSR | HOM-TES-85 [59,78] | 17 | 2044 | VKELLKGQSRLIFTYGLINS | MPHOSPH1 |
| | | | | | | | [137,156] |
| 5 | 652 | LLLSLHGVAASLEVSESPGS | IGES11 [12,31] | 17 | 2045 | PPLTSSNHSTSPQLSTGVSF | MUC-1 |
| | | | | | | | [1023,1042] |
| 5 | 653 | DGFDLNKGIEAKIHTLLPWQ | IL13RA2 [93,112] | 17 | 2046 | EVLFYQPDLKYLSFILTKLS | NLRP4 |
| | | | | | | | [688,707] |
| 5 | 654 | EGRALRYMIERTVNWQHRAQ | JARID1B | 17 | 2047 | KENFTGSETLKHLVLQFLQQ | NXF2 [379,398] |
| | | | [1248,1267] | | | | |
| 5 | 655 | RNIPPHLQWEVLDSLLVQYG | KOC1 [86,105] | 17 | 2048 | QEKENKYFEDIKILKEKNAE | NY-BR-1 |
| | | | | | | | [1070,1089] |
| 5 | 656 | DVGYGRSISSSSSLRRSSKE | KU-CT-1 [621,640] | 17 | 2049 | SWDVDDLLLWKKIHRMVILT | NYD-TSPG |
| | | | | | | | [333,352] |
| 5 | 657 | SRLLQLHITMPFSSPMEAEL | Lage-1 [85,104] | 17 | 2050 | EKSEEYAEQLHVQLADKELY | ODF2 [414,433] |
| 5 | 658 | LKGEMMDLQHGSLFFSTSKI | LDHC [58,77] | 17 | 2051 | QKQPNTLRHVVIPELQSSEA | PASD1 |
| | | | | | | | [410,429] |
| 5 | 659 | QTIESWREEGFPVGLKLAVL | LEMD1 [142,161] | 17 | 2052 | RGTTEIGMIGSKPFSTVKYK | PEPP2 |
| | | | | | | | [779,798] |
| 5 | 660 | TYKIQRLMLKSLTYPERPPL | LIPI [437,456] | 17 | 2053 | KSIGRGYNPRLTVIVVKKRV | PIWIL1 |
| | | | | | | | [725,744] |
| 5 | 661 | QSPQGASAFPTTINFTRQRQ | MAGE-A1 [56,75] | 17 | 2054 | YHDPSRGMRSVVGFVASINL | PIWIL2 |
| | | | [747,766] | | | | |
| 5 | 662 | PRAHAEIRKMSLLKFLAKVN | MAGE-A10 | 17 | 2055 | SPNNFTLAFIVVKKRINTRF | PIWIL3 |
| | | | [300,319] | | | | [750,769] |
| 5 | 663 | EESFSPTAMDAIFGSLSDEG | MAGE-A11 | 17 | 2056 | MSGRARVKARGIARSPSATE | PIWIL4 [1,20] |
| | | | [177,196] | | | | |
| 5 | 664 | DGREDSVFAHPRKLLTQDLV | MAGE-Al2 | 17 | 2057 | DVLLAQEVRPRRWKLQVLDL | PRAME |
| | | | [232,251] | | | | [105,124] |
| 5 | 665 | AISRKMVELVWFLLLKYRAR | MAGE-A2 [108,127] | 17 | 2058 | KFSGYPLYHSVYETYELVEK | PSMA [545,564] |
| 5 | 666 | KLLTQHFVQENYLEYRQVPG | MAGE-A3 [244,263] | 17 | 2059 | RSVYSKQPYTEYISTRWYRA | RAGE-1 |
| | | | | | | | [150,169] |
| 5 | 667 | LLIIVLGTIAMEGESASEEE | MAGE-A4 [202,221] | 17 | 2060 | KMENVQPAPENVLLTLRPRR | SAGE1 |
| | | | | | | | [210,229] |
| 5 | 668 | LDTQEEALGLVGVQAATTEE | MAGE-A5 [16,35] | 17 | 2061 | LALWEAYREFESAIVEAARL | SART3 |
| | | | | | | | [225,244] |
| 5 | 669 | FPVIFSKASDSLQLVFGIEL | MAGE-A6 [147,166] | 17 | 2062 | TEKENKMKELTELLEESREK | SCP-1 [276,295] |
| 5 | 670 | QEALKIKVAELVHELIHKYR | MAGE-A9 [105,124] | 17 | 2063 | EYSQQFLTLFQQWDLDMQKA | SCP3a [124,143] |
| 5 | 671 | STESSVKDPVAWEAGMLMHE | MAGE-B1 [99,118] | 17 | 2064 | AHEWARAIIESDQEQGRKLR | SCRN1 |
| | | | | | | | [350,369] |
| 5 | 672 | KIVGKRFREHFPEILKKASE | MAGE-B2 [139,158] | 17 | 2065 | QDLQREISILQEQISHLQFV | se57-1 |
| | | | | | | | [230,249] |
| 5 | 673 | TVPSAFQFWYEEALRBEEER | MAGE-B3 [298,317] | 17 | 2066 | IKEGLITADAEGDFIDARPG | SLCO6A1 |
| | | | | | | | [550,569] |
| 5 | 674 | KEVNPTTHSYILVSMLGPND | MAGE-B4 [167,186] | 17 | 2067 | PQDTVMKAIQEARKMREQIQ | SOX-6 [492,511] |
| 5 | 675 | DEESVSASQKAIIEKRLSKD | MAGE-B6 [174,193] | 17 | 2068 | QLANDSVNRLSRILMELDGP | SPAG1 |
| | | | | | | | [555,574] |
| 5 | 676 | PVSPSFSSTLVSLEQSSPER | MAGE-C1 [271,290] | 17 | 2069 | DVLQGELEAVKQAKLKLEEK | SPAG9 |
| | | | | | | | [446,465] |
| 5 | 677 | ESESSFTYTLEEKVAELVEF | MAGE-C2 [132,151] | 17 | 2070 | TAVAVPSNIQGIRNLVTDAK | SPO11 |
| | | | | | | | [199,218] |
| 5 | 678 | STTHSFLFGALAELLDNARD | MORC1 [22,41] | 17 | 2071 | SEEYLERQLQAEFIESGQYR | TAF7L [363,382] |
| 5 | 679 | SSKKTYSLRSQASIIGVNLA | MPHOSPH1 | 17 | 2072 | KWDPVVPFRNESAVTGYKML | TAG-1 |
| | | | [1703,1722] | | | | [931,950] |
| 5 | 680 | LQRDISEMFLQIYKQGGFLG | MUC-1 [1069,1088] | 17 | 2073 | KYTSEDFWYRAVVLGTSETE | TDRD1 |
| | | | | | | | [1000,1019] |
| 5 | 681 | AETEPERHLGSYLLDSENTS | NKX3.1 [91,110] | 17 | 2074 | YRYLNSWRPSLFYKIANVQT | TEKT5 [44,63] |
| 5 | 682 | RNKSVRYLDLSANVLKDEGL | NLRP4 [805,824] | 17 | 2075 | TGLKRLSSFIGAGSPSLVKA | TEX14 |
| | | | | | | | [1283,1302] |
| 5 | 683 | STWQELILLSSLTVYSKQIF | NR6A1 [322,341] | 17 | 2076 | NDKNSKVLMQNAATYWNELP | TEX15 |
| | | | | | | | [2618,2637] |
| 5 | 684 | QMEMLKLTMNKRYNVSQQAL | NXF2 [213,232] | 17 | 2077 | RKRRRRRRLMELAEPKINWQ | THEG [120,139] |
| 5 | 685 | ITKRSEQIVEFLLIKNANAN | NY-BR-1 [124,143] | 17 | 2078 | QVKHLYNWNLPPRRILFIKH | TPTE [399,418] |
| 5 | 686 | LVERVDETTPAVVQSVILER | NYD-TSPG [87,106] | 17 | 2079 | IKLESSKELLNRQLVAKDQE | TSGA10 |
| | | | | | | | [516,535] |
| 5 | 687 | LELEIIVLNDRVTDLVNQQQ | ODF2 [458,477] | 17 | 2080 | ADGMWPQFRTIQEKEVIILN | TSP50 [250,269] |
| 5 | 688 | SPGPRYNVNPKILRTGKDLG | ODF3 [65,84] | 17 | 2081 | FFNWFSDHNFAGSNKIAEIL | TSPY1 [257,276] |
| 5 | 689 | AFSKKWLDLSRSLFYQRWPV | ODP4 [99,118] | 17 | 2082 | PWHRLELLRWEQEIQKLTGD | TYR [209,228] |
| 5 | 690 | YLLKTKAIVNASEMDIQNVP | OIP5 [172,191] | 17 | 2083 | LQPVETKAHETSSEPQLLWD | ZNF165 |
| | | | | | | | [163,182] |
| 5 | 691 | ILRTQLLQQLYTSKAVSDEA | PASD1 [175,194] | 18 | 2084 | YLPREVLAQLPSLRHLDLSN | 5T4 [224,243] |
| 5 | 692 | VYQKRLMDEAKILKSLHHPN | PBK [77,96] | 18 | 2085 | SIIKEGIESQASYKIVIEGK | ADAM2 [35,54] |
| 5 | 693 | WQQRQFYNKQSTLPRHSTLS | PEPP2 [506,525] | 18 | 2086 | KVLLFGLEIWTNKNLIVVED | ADAM29 |
| | | | | | | | [242,261] |
| 5 | 694 | RRSIAGEVASINEGMTRWFS | PIWIL1 [641,660] | 18 | 2087 | EGLSPNLNRFLGERDFITQFS | AFP [324,343] |
| 5 | 695 | KWYSRVVFQMPHQEIVDSLK | PIWIL2 [770,789] | 18 | 2088 | AQDKAESYSLISMKGMGDPK | AKAP-3 |
| | | | | | | | [397,416] |
| 5 | 696 | LHSWLILYSRSSHREAMSLK | PIWIL3 [507,526] | 18 | 2089 | RNQSLEFSTMKAEMKERDKG | AKAP-4 |
| | | | | | | | [461,480] |
| 5 | 697 | QENPAAFVRAIQQYVDPDVQ | PIWIL4 [526,545] | 18 | 2090 | AKGRRAEDGSVIDYELIDQD | ANXA2 |
| | | | | | | | [175,194] |
| 5 | 698 | HQAGAQEAQPLQPSHELDIS | PLAC1 [179,198] | 18 | 2091 | PSVFPKTSISPLNVVQGASV | BRDT [178,197] |
| 5 | 699 | SLLKDEALAIAALELLPREL | PRAME [33,52] | 18 | 2092 | YRGNTTMDIKELVKQFHQIK | CABYR [47,66] |
| 5 | 700 | GDRSIFEGRTRYSRITGGME | PRSSSS [54,73] | 18 | 2093 | ERPEIVSTWSSAGISWRSEA | CAGE1 |
| | | | | | | | [260,279] |
| 5 | 701 | STNEVTRIYNVIGTLRGAVE | PSMA [348,367] | 18 | 2094 | TQNGRFYAISARFKPFSNKG | CALR3 [69,88] |
| 5 | 702 | VEYEIASLQPLFPGVNELDQ | RAGE-1 [191,210] | 18 | 2095 | QERTNSELHNLRQIYVKQQS | CCDC62 |
| | | | | | | | [275,294] |
| 5 | 703 | QQRKKMEKEAQRLMKKEQNK | RCAS1 [188,207] | 18 | 2096 | YAKIDEKTAELKRKMFKMST | CDCA1 |
| | | | | | | | [445,464] |
| 5 | 704 | QSRTDKVLSTAPPQLVHMAA | SAM [121,140] | 18 | 2097 | EEKLSKVDEMISHATEELET | CTAGE2 |
| | | | | | | | [381,400] |
| 5 | 705 | NAKYANMWLEYYNLERAHGE | SART3 [504523] | 18 | 2098 | LLLEELRRVVAALPEGMRPD | CTAGE5 [14,33] |
| 5 | 706 | MEESNKARAAHSFVVTEFET | SCP-1 [363,382] | 18 | 2099 | QEGVQVVVQQPGPGLLWLEE | CTCPL [105,124] |
| 5 | 707 | KILQQSRIVQSQRLKTIKQL | SCP3a [159,178] | 18 | 2100 | PKSLEIRTLTVERLLEPLVT | CTNNA2 [14,33] |
| 5 | 708 | GETAKEALDVIVSLLEEHGQ | SCRN1 [120,139] | 18 | 2101 | NTLFVVDVQTSQITKIPILK | DCAF12 |
| | | | | | | | [110,129] |
| 5 | 709 | IEETEYVKKIRTTLQKIRTQ | se57-1 [31,50] | 18 | 2102 | AAPIHDADAQESSLGLTGLQ | DKKL1 [30,49] |
| 5 | 710 | EKSYDISSGLVAIFIAFYGD | SLCO6A1 [148,167] | 18 | 2103 | GKIPIRWTAPEAISYRKFTS | EPHA2 |
| | | | | | | | [777,796] |
| 5 | 711 | SYNHKQIEQLYAAQLASMQV | SOX-6 [360,379] | 18 | 2104 | LKFVSSLRRQFEFSVDSFQI | FAM46D |
| | | | | | | | [159,178] |
| 5 | 712 | ISGKEEETSVTILDSSEEDK | SP17 [92,111] | 18 | 2105 | AKLARQATNLKVNFFFERIM | FBXO39 |
| | | | | | | | [274,293] |
| 5 | 713 | SGDYEEAVMYYTRSISALPT | SPAG1 [222,241] | 18 | 2106 | TQIPPEEYEMQMQKLNKDFT | FSIP1 [202,221] |
| 5 | 714 | GREVENLILENTQLLETKNA | SPAG9 [407,426] | 18 | 2107 | IAKRNKHHEIFNLLSFTLNP | GASZ [221,240] |
| 5 | 715 | IDVVESEAVSVLHHWLKKTE | SRATA19 [28,47] | 18 | 2108 | NYTNAMFKNNYPSLTPQAFE | Glypican-3 |
| | | | | | | | [124,143] |
| 5 | 716 | YATKRDIYYTDSQLFGNQTV | SPO11 [130,149] | 18 | 2109 | VNNTIINGSQVWGGQPVYPQ | gp100 |
| | | | | | | | [105,124] |
| 5 | 717 | HRLRERKQLVIYEEISDPEE | SSX-1 [166,185]; | 18 | 2110 | KTGKVRILIATELASRGLDV | HAGE [534,553] |
| | | | SSX-2 [166,185] | | | | |
| 5 | 718 | QIPEKIQKAFDDIAKYFSKE | SSX-2 [16,35] | 18 | 2111 | DVDKLHFTPRIQRLNELEAQ | JARID1B [77,96] |
| 5 | 719 | PGKPTTSEKINMISGVLQRY | SSX-3 [142,161] | 18 | 2112 | GPPEAQFKAQGRIYGKIKEE | KOC1 [458,477] |
| 5 | 720 | GKHAWTHRLRERKQLVVYEE | SSX-4 [160,179] | 18 | 2113 | LANMSAEYTSKVQIFEHGGL | KU-CT-1 |
| | | | | | | | [133,152] |
| 5 | 721 | ATLPPFMRNKRVADFQGNDF | SSX-5 [65,84] | 18 | 2114 | GTDSDKEHWKNIHKQVIQSA | LDHC [219,238] |
| 5 | 722 | AFLSVKKQFEELTLGEFLKL | Survivin [85,104] | 18 | 2115 | LEFSQLSVKDSFRELFIPRI | LIPI [43,62] |
| 5 | 723 | AEGPSTLDEGLFLRSQEAAA | SYCE1 [219,238] | 18 | 2116 | ALAETSYVKVLEYVIKVSAR | MAGE-A1 |
| | | | | | | | [270,289] |
| 5 | 724 | DEVPDEVENQFILRLPLEHA | TAF7L [92,111] | 18 | 2117 | ESVIRNYEDHFPLLFSEASE | MAGE-A10 |
| | | | | | | | [162,181] |
| 5 | 725 | NESAVTGYKMLYQNDLHLTP | TAG-1 [940,959] | 18 | 2118 | RAHAETSKMKVLEYIANANG | MAGE-A11 |
| | | | | | | | [389,408] |
| 5 | 726 | DKTIQANVLEIISPNLFYAL | TDRD1 [939,958] | 18 | 2119 | PDAESLFREALSNKVDELAH | MAGE-A4 |
| | | | | | | | [100,119] |
| 5 | 727 | DNIKHSQNMRANSIQLREEA | TEKT5 [301,320] | 18 | 2120 | DLESEFQAALSRKVAKLVHF | MAGE-A6 |
| | | | | | | | [100,119] |
| 5 | 728 | QGFIHRSLSSYAVHIISPGE | TEX14 [370,389] | 18 | 2121 | EEIWKFMNVLGAYDGEEHLI | MAGE-B1 |
| | | | | | | | [218,237] |
| 5 | 729 | VTELEYNYNQFSTLLKNVMS | TEX15 [2198,2217] | 18 | 2122 | RGQKSKLRAREKRRKARDET | MAGE-B2 [3,22] |
| 5 | 730 | PKIRDNFWSMPMSEVSQVSR | THEG [238,257] | 18 | 2123 | EFLNKMRIYDGKKHFIFGEP | MAGE-B3 |
| | | | | | | | [225,244] |
| 5 | 731 | SFYFWLHTSFIENNRLYLPK | TPTE [493,512] | 18 | 2124 | GEPRKLITQDLVQEKYLEYQ | MAGE-B4 |
| | | | | | | | [239,258] |
| 5 | 732 | VLGEAWRDQVDWSRLLRDAG | TRAG-3 [45,64] | 18 | 2125 | DDSTATESASSSVMSPSFSS | MAGE-C1 |
| | | | | | | | [1122,1141] |
| 5 | 733 | KARQSEADNNTLKLELITAE | TSGA10 [430,449] | 18 | 2126 | LLVIYNLKLLLNGEPELDVK | MORC1 |
| | | | | | | | [197,216] |
| 5 | 734 | DPEAVARRWPWMVSVRANGT | TSP50 [116,135] | 18 | 2127 | LPRTLNVLETSLQERLYTKM | MPHOSPH1 |
| | | | | | | | [172,191] |
| 5 | 735 | FWANVIANHPQMSALITEED | TSPY1 [158,177] | 18 | 2128 | LFEMQDPAFVKQAVNLLQEA | NLRP4 |
| | | | | | | | [557,576] |
| 5 | 736 | DSFQDYIKSYLEQASRIWSW | TYR [458,477] | 18 | 2129 | FFVQBASAASALKDVSYKIY | NXF2 [166,185] |
| 5 | 737 | GATLKGVAAGSSSSVKWTEG | WT1 [244,263] | 18 | 2130 | KKEIAMLKLEIATLKHQYQE | NY-BR-1 |
| | | | | | | | [1052,1071] |
| 5 | 738 | WRGRSTYRPRPRRSLQPPEL | XAGE-2 [3,22] | 18 | 2131 | LTFVMPNDYTKEVAEYFQER | NYD-TSPG |
| | | | | | | | [180,199] |
| 5 | 739 | GKTFRVSSHLIRHFRIHTGE | ZNF165 [434,453] | 18 | 2132 | EMDGAAAAKQVMALKETIGK | ODF2 [215,234] |
| 6 | 740 | LQGLRRLELASNHFLYLPRD | 5T4 [209,228] | 18 | 2133 | KDEVYQKIILKFPLLNSETH | RASD1 [86,105] |
| 6 | 741 | EDVRVSGWLQTEFLSFQDGD | ACRBP [452,471] | 18 | 2134 | NLRENPFRTTQTRRREDKEL | PEPP2 |
| | | | | | | | [940,959] |
| 6 | 742 | MQRVAPEMFFSPQVFEQPGP | ACTL8 [251,270] | 18 | 2135 | QETAQLVGSTASQQPGYIQP | PIWIL1 [16,35] |
| 6 | 743 | GLGGLRMDSNETSLPVQITV | ADAM2 [10,29] | 18 | 2136 | AMDQARELVNMLEKIAGPIG | PIWIL2 |
| | | | | | | | [614,633] |
| 6 | 744 | LQINDFAYEIKPLAFSTTFE | ADAM29 [127,146] | 18 | 2137 | SHREAMSLKGHLQSVTAPMG | PIWIL3 |
| | | | | | | | [518,537] |
| 6 | 745 | DLATIFFAQFVQEATYKEVS | AFP [45,64] | 18 | 2138 | ARLVDNIQRNTNARFELETW | PIWIL4 |
| | | | | | | | [415,434] |
| 6 | 746 | NRNVNFAMKSETKLREKMYS | AKAP-3 [417,436] | 18 | 2139 | SQLTTLSFYGNSISISALQS | PRAME |
| | | | | | | | [406,425] |
| 6 | 747 | SKGLMVYANQVASEMMVSLM | AKAP-4 [330,349] | 18 | 2140 | WNLLHETESAVATARRPRWL | PSMA [2,21] |
| 6 | 748 | SETSGEFRKLMVALAKGRRA | ANXA2 [161,180] | 18 | 2141 | GEGTFSEVMKMQSLREGNYY | RAGE-1 [11,30] |
| 6 | 749 | VDAVKLQLPDYYTIIKNPMD | BRDT [56,75] | 18 | 2142 | MSTRDQYAAVTHNIREEKIN | SAGE1 |
| | | | | | | | [616,635] |
| 6 | 750 | SKPRLVVPYGLKTLLEGISR | CABYR [4,23] | 18 | 2143 | LERQLKVKDLVLSVHNRAIR | SART3 |
| | | | | | | | [354,373] |
| 6 | 751 | YHSLNEELDFLVTSYEEIIE | CAGE1 [694,713] | 18 | 2144 | QENRKIIEAQRKAIQELQFG | SCP-1 [135,154] |
| 6 | 752 | AISARFKPFSNKGKTLVIQY | CALR3 [76,95] | 18 | 2145 | MFRQQQKILQQSRIVQSQRL | SCP3a [153,172] |
| 6 | 753 | DKELNDMVAVHQQQLLSWEE | CCDC62 [41,60] | 18 | 2146 | SLELLMKKIKGKELQLLEMN | se57-1 [87,106] |
| 6 | 754 | LKTEENSFKRLMIVKKEKLA | CDCA1 [334,353] | 18 | 2147 | EPHIKRPMNAFMVWAKBERR | SOX-6 [618,637] |
| 6 | 755 | QLQVQREIVNKHNELRKAVS | CRGP2 [33,52] | 18 | 2148 | IIEAKMELEEVTRLLNLKDK | SPAG1 |
| | | | | | | | [724,743] |
| 6 | 756 | AMSKAKKLMTGHAIPPSQLD | CT45 [51,70] | 18 | 2149 | SVTKAERSHLIVWQVMYGNE | SPAG9 |
| | | | | | | | [1302,1321] |
| 6 | 757 | SNESSMLSTETKKASILLIR | CT46 [136,155] | 18 | 2150 | ITGKGVPDLNTRLLVKKLWE | SPO11 |
| | | | | | | | [247,266] |
| 6 | 758 | EEGEQAAGLAAVPRGGSAEE | CT47 [64,83] | 18 | 2151 | PLKNVRKKRFRKTQKKVPDV | TAF7L [225,244] |
| 6 | 759 | TELYQENEMKLYRKLIVEEK | CTAGE2 [356,375] | 18 | 2152 | SSSLSIKWEPVVPFRNESAV | TAG-1 |
| | | | | | | | [925,944] |
| 6 | 760 | SKSLKSQVAEAKMTFKIFQM | CTAGE5 [180,199] | 18 | 2153 | QKKAHVLYIDYGNEEIIPLN | TDRD1 |
| | | | | | | | [341,360] |
| 6 | 761 | NMAFVTSGELVRHRRYKHTH | CTCFL [319,338] | 18 | 2154 | ELERLLTENQNLETVKRRLE | TEKT5 [158,177] |
| 6 | 762 | RGSQKKHISPVQALSEFKAM | CTNNA2 [931,950] | 18 | 2155 | FEDWEWQNGSLSSLSLPEST | TEX14 |
| | | | | | | | [696,715] |
| 6 | 763 | VTSINEDNIYISNSIYFSIA | CXorf48 [118,137] | 18 | 2156 | QHSVEYEGNIHTSLAIAQKL | TEX15 |
| | | | | | | | [343,362] |
| 6 | 764 | NSTALALVRPSSSGLINSNT | CXorf61 [34,53] | 18 | 2157 | AAQMAVPSSRILQLSKPKAP | THEG [258,277] |
| 6 | 765 | HQTAIKRNNPRKFLRSVGDG | DBPC [122,141] | 18 | 2158 | DIKLLRNIPRWTHLLRLLRL | TPTE [180,199] |
| 6 | 766 | SRVPVYAHITHKALKDIPKE | DCAF12 [227,246] | 18 | 2159 | EESENRQMMEQLRKANEDAE | TSGA10 |
| | | | | | | | [406,425] |
| 6 | 767 | STLVIPSAAAPIHDADAQES | DKKL1 [22,41] | 18 | 2160 | SAKEGTAFRMEAVQEGAAGV | TSPY1 [33,52] |
| 6 | 768 | QSVPQFFTFEDAPSYPEARA | DMRT1 [302,321] | 18 | 2161 | TERRLLVRRNIFELSAPEKE | TYR [113,132] |
| 6 | 769 | GRLLSADTKGWVRLQFHAGQ | DPPA2 [228,247] | 18 | 2162 | AQBEGFGKILTHKNTVRGEI | ZNF165 |
| | | | | | | | [250,269] |
| 6 | 770 | FITSILYENNVITIBLVQNS | EpCAM [180,199] | 19 | 2163 | SSSAPFLASAVSAQPPLPBQ | 5T4 [42,61] |
| 6 | 771 | AISYRKFTSASEVWSFGIVM | EPHA2 [788,807] | 19 | 2164 | GLRGVLQFENVSYGIEPLES | ADAM2 |
| | | | | | | | [113,132] |
| 6 | 772 | SVESFQIVLDPMLEFYSDKN | FAM46D [172,191] | 19 | 2165 | EIPNMSEHTTVHWARFNEIM | ADAM29 |
| | | | | | | | [561,580] |
| 6 | 773 | PGTDAVAQTSLEEFNVLEME | FATE1 [116,135] | 19 | 2166 | VMKNFGTRTFQAITVTKLSQ | AFP [226,245] |
| 6 | 774 | MGKRLDYLNLKGARLTVEQG | FBXO39 [160,179] | 19 | 2167 | GRREARSFVEAAGTTNFPAN | AKAP-3 |
| | | | | | | | [543,562] |
| 6 | 775 | KRKRKSEMLQKAARGREEHG | FMR1NB [234,253] | 19 | 2168 | NRLSSLVIQMAHKEIKEKLE | AKAP-4 |
| | | | | | | | [222,241] |
| 6 | 776 | KRPSFLDEPLYGISVSLSSE | ESIP1 [533,552] | 19 | 2169 | LYYYIQQDTKGDYQKALLYL | ANXA2 |
| | | | | | | | [315,334] |
| 6 | 777 | HFLESHYLHEQVKTIKELGG | FTHL17 [132,151] | 19 | 2170 | SSEELFNQFRKAAIEKEVKA | BRDT [821,840] |
| 6 | 778 | HHEIFNLLSFTLNPLEGKLQ | GASZ [227,246] | 19 | 2171 | IKIGSEKSLHLEVEITSIVS | CABYR |
| | | | | | | | [409,428] |
| 6 | 779 | EQPRWVSHHHPAVLNGQHPB | GATA-3 [6,25] | 19 | 2172 | ESRNEKEMLQLQFKKIKANY | CAGE1 |
| | | | | | | | [398,417] |
| 6 | 780 | DMVNELFESLFPVIYTQLMN | Glypican-3 | 19 | 2173 | KIDGQSIESGSIEYDWNLTS | CALR3 |
| | | | [165,184] | | | | [185,204] |
| 6 | 781 | VLYRYGSFSVTLDIVQGIES | gp100 [476,495] | 19 | 2174 | NQKSLFKBQKFEAMLVQQNR | CCDC62 |
| | | | | | | | [348,367] |
| 6 | 782 | MSSTEKVIVFVSRKAVADHL | HAGE [484,503] | 19 | 2175 | AHQEALMKLERLDSVPVEEQ | CDCA1 |
| | | | | | | | [158,177] |
| 6 | 783 | ELLKYHQRVLYIDIDIHHGE | HDAC1 [162,181]; | 19 | 2176 | SENQKLQQKLKVMTELYQEN | CTAGE2 |
| | | | HDAC2 [163,182] | | | | (343,362) |
| 6 | 784 | FKPIISKVMEMYQPSAVVLQ | HDAC2 [242,261] | 19 | 2177 | MKLHRKLTVEENYRLEKEEK | CTAGE5 |
| | | | | | | | [394,413] |
| 6 | 785 | QNSRQYLDQIRQTIFENLKM | HDAC3 [349,368] | 19 | 2178 | SYASRETYKLKRHMRTHSGE | CTCFL [376,395] |
| 6 | 786 | GDLVDTQSRLIATQRDLIAT | HOM-TES-85 | 19 | 2179 | QVEVAIEALSANVPQPFEEN | CTNNA2 |
| | | | [236,255] | | | | [588,607] |
| 6 | 787 | TGTMPATNVSIFINNTQLSR | IGFS11 [95,114] | 19 | 2180 | KSDARHNVSRVPVYAHITHK | DCAF12 |
| | | | | | | | [219,238] |
| 6 | 788 | EIKVNPPQDFEIVDPGYLGY | IL13RA2 [29,48] | 19 | 2181 | HTELHPRVAFWIIKLPRRRS | DKKL1 |
| | | | | | | | [160,179] |
| 6 | 789 | IEEIPAYLPNGAALKDSVQR | JARID1B [997,1016] | 19 | 2182 | GTNFQKRLFTKIDTIAPDEI | EPHA2 |
| | | | | | | | [131,150] |
| 6 | 790 | LEKLNGFQLENFTLKVAYIP | KOC1 [136,155] | 19 | 2183 | KESYPVVVAESMYGDFQEAM | FAM46D |
| | | | | | | | [196,215] |
| 6 | 791 | KKNSYHFSAGEGSPIEDKSE | KU-CT-1 [643,662] | 19 | 2184 | NPYNAVLTKKFQVTMRGLLS | FBXO39 |
| | | | | | | | [98,117] |
| 6 | 792 | GARRPDSRLLQLHITMPFSS | Lage-1 [79,98] | 19 | 2185 | AWQSKEEMENTKKFLSLTAV | FSIP1 [162,181] |
| 6 | 793 | SILKNLRRVHPVSTMVKGLY | LDHC [262,281] | 19 | 2186 | GAEVNTQDENGYTALTWAAR | GASZ [172,191] |
| 6 | 794 | LSTEKSKKLKKWPEASTTKR | LEMD1 [80,99] | 19 | 2187 | KVEGNERKLIMTQVSKSLQV | Glypican-3 |
| | | | | | | | [206,225] |
| 6 | 795 | SRGATTFIYNRAVKNTRKVA | LIPI [135,154] | 19 | 2188 | FSVPQLPHSSSHWLRLPRIF | gp100 |
| | | | | | | | [625,644] |
| 6 | 796 | ITKKVADLVGFILLKYRARE | MAGE-A1 [102,121] | 19 | 2189 | LKNITYLVLDEADKMLEMGF | HAGE [387,406] |
| 6 | 797 | QDRIATTDDTTAMASASSSA | MAGE-A10 | 19 | 2190 | QTRVKLNELDQTAKYWELQG | JARID1B |
| | | | [342,361] | | | | [96,115] |
| 6 | 798 | GSVIKNYEDYFPEIFREASV | MAGE-A11 | 19 | 2191 | HSVPKRQRIRKLQIRNIPPH | KOC1 [72,91] |
| | | | [250,269] | | | | |
| 6 | 799 | KASEYLQLVEGIEVVEVVRI | MAGE-A12 | 19 | 2192 | GSKEFFNNQGIPQLIQLLKS | KU-CT-1 |
| | | | [153,172] | | | | [348,367] |
| 6 | 800 | SKASEYLQLVFGIEVVEVVP | MAGE-A2 [152,171] | 19 | 2193 | MSTVKEQLIEKLIEDDENSQ | LDHC [1,20] |
| 6 | 801 | EEQEAASSSSTLVEVTLGEV | MAGE-A3 [34,53]; | 19 | 2194 | AKFVEVIHSESNGLGIQEPL | LIPI [224,243] |
| | | | MAGE-A6 [34,53] | | | | |
| 6 | 802 | ATEEQEAASSSSTLVEVTLG | MAGE-A6 [32,51] | 19 | 2195 | HAEIRKMSLLKFLAKVNGSD | MAGE-A10 |
| | | | | | | | [303,322] |
| 6 | 803 | VRVNARVRISYPSLHEEALG | MAGE-A8 [294,313] | 19 | 2196 | VYAGREHFLFGEPKRLLTQN | MAGE-A11 |
| | | | | | | | [343,362] |
| 6 | 804 | VGKEHMFYGEPRKLLTQRWV | MAGE-A9 [231,250] | 19 | 2197 | KISGGPRISYPLLHEWALRE | MAGE-A6 |
| | | | | | | | [292,311] |
| 6 | 805 | MHFILRKYKMRERIMKADML | MAGE-B1 [116,135] | 19 | 2198 | RSSVRARRRTTATTFRARSR | MAGE-B1 |
| | | | | | | | [318,337] |
| 6 | 806 | ESLLSSWEEPRRKLLMPLLG | MAGE-B2 [190,209] | 19 | 2199 | VELTDEESLLSSWEEPRRKL | MAGE-B2 |
| | | | | | | | [184,203] |
| 6 | 807 | VQFLMEMYKMKKRIMKADML | MAGE-B3 [119,138] | 19 | 2200 | RGQTQDHQGAQITATNKKKV | MAGE-B3 |
| | | | | | | | [19,38] |
| 6 | 808 | FREIFRKVSQRTELVFGLAL | MAGE-B4 [147,166] | 19 | 2201 | AEKEESPSSSSSVLRETASS | MAGE-B4 |
| | | | | | | | [33,52] |
| 6 | 809 | ESSGESYTTVSKTGLPSEGI | MAGE-B6 [256,275] | 19 | 2202 | RTHSTFEGFPQSLLQIPMTS | MAGE-C1 |
| | | | | | | | [325,344] |
| 6 | 810 | EFLAMLKNTVPITFPSSYKD | MAGE-C1 | 19 | 2203 | PMEDVNLSSGHIARVSVSGS | MORC1 |
| | | | [1086,1105] | | | | [780,799] |
| 6 | 811 | RRELLTKVWVQGHYLEYREV | MAGE-C2 [270,289] | 19 | 2204 | ENNEGLRAFLLTIENELKNE | MPHOSPH1 |
| | | | | | | | [882,901] |
| 6 | 812 | EKPLNSFQYQRRQAMGIPFI | MORC1 [464,483] | 19 | 2205 | YQRDLKYLSFTLTKLSRDDI | NLRP4 |
| | | | | | | | [692,711] |
| 6 | 813 | RAKRKLYTSEISSPIDISGQ | MPHOSPH1 | 19 | 2206 | TIPYGIKYDKAWLMNSIQSH | NXF2 [128,147] |
| | | | [1775,1794] | | | | |
| 6 | 814 | TSPQLSTGVSFEELSFHISN | MUC-1 [1032,1051] | 19 | 2207 | LQSKNMWLQQQLVHAHKKAD | NY-BR-1 |
| | | | | | | | [1270,1289] |
| 6 | 815 | TETQVKIWFQNRRYKTKRKQ | NKX3.1 [164,183] | 19 | 2208 | RGKAPDPQAGNEVLVFPFNE | NYD-TSPG |
| | | | | | | | [545,564] |
| 6 | 816 | STTSVYSSFVFNLFTPEGAE | NLRP4 [367,386] | 19 | 2209 | HKAEVEAIMEQLKELKQKGD | ODF2 [377,396] |
| 6 | 817 | RSLDPQSYSLIHQLLSAEDL | NR6A1 [244,263] | 19 | 2210 | HDAIQNQQNALELMMEHLQK | PASD1 |
| | | | | | | | [392,411] |
| 6 | 818 | DNEWNYTRAGQAFTMLQTEG | NXF2 [596,615] | 19 | 2211 | RSWTYGITRGGRVFFINEEA | PEPP2 [14,33] |
| 6 | 819 | SSEIVGMLLQQNVEVFAADI | NY-BR-1 [161,180] | 19 | 2212 | GLITKMRNDENVMKELAVHT | PIWIL1 |
| | | | | | | | [391,410] |
| 6 | 820 | DEMYIVQKYISNPLLIGRYK | NYD-TSPG [234,253] | 19 | 2213 | FNYQPKMVVFVVQKKISTNL | PIWIL2 |
| | | | | | | | [841,860] |
| 6 | 821 | ATSAQNIEFLQVIAKREFAI | ODF2 [675,694] | 19 | 2214 | FRRTFKLLDFEQVGRNYYTK | PIWIL3 |
| | | | | | | | [229,248] |
| 6 | 822 | PAAYRQTDVRVTKFKAPQYT | ODF3 [182,201] | 19 | 2215 | SATEVGRIQASPLPRSVELS | PIWIL4 [17,36] |
| 6 | 823 | NIWIFELERNVSIPIGNSYF | ODF4 [165,184] | 19 | 2216 | MPMQDIKMILKMVQLDSIED | PRAME |
| | | | | | | | [218,237] |
| 6 | 824 | NNVVLEAPFLVGIEGSLKGS | OIP5 [111,130] | 19 | 2217 | FLDELKAFNIKKFLYNFTQI | PSMA [61,80] |
| 6 | 825 | RADPVDLEFSVDQVDSVDQE | PASD1 [298,317] | 19 | 2218 | TPAQKILTKFKQSRAMNFDF | RAGE-1 |
| | | | | | | | [220,239] |
| 6 | 826 | FDDEAYYAALGTRPPINMEE | PBK [266,285] | 19 | 2219 | NNSQPAPGNILSTAPPWLRH | SAGE1 |
| | | | | | | | [635,654] |
| 6 | 827 | SQEIEMHADNPAAIQTVVLQ | PEPP2 [674,693] | 19 | 2220 | ESDGDEYAMASSAESSPGEY | SART3 [64,83] |
| 6 | 828 | IYTRRNYEAANSLIQNLFKV | PIWIL1 [499,518] | 19 | 2221 | ELEDIKVSLQRSVSTQKALE | SCP-1 [323,342] |
| 6 | 829 | AASIRRTEGGLFLLADVSHK | PIWIL2 [364,383] | 19 | 2222 | SILQEQISHLQFVIHSQHQN | se57-1 |
| | | | | | | | [237,256] |
| 6 | 830 | KKKATQLYRHGTSLEINLGY | PIWIL3 [248,267] | 19 | 2223 | EKQPYYEEQARLSKIHLEKY | SOX-6 [666,685] |
| 6 | 831 | SNKAKAFDGAILFLSQKLEE | PIWIL4 [151,170] | 19 | 2224 | RALRKDKPAATAASFTAFEW | SPAG1 [73,92] |
| 6 | 832 | QLTTLSFYGNSISISALQSL | PRAME [407,426] | 19 | 2225 | GVEGQWELSNYHLLDLGRPH | SPAG9 |
| | | | | | | | [1028,1047] |
| 6 | 833 | EAEVGEFPWQVSIQARSEPF | PRSS55 [73,92] | 19 | 2226 | PMGPEASFFDVLDRHRESLL | SPO11 [5,24] |
| 6 | 834 | SFDSLFSAVKNFTEIASKFS | PSMA [628,647] | 19 | 2227 | NEVKRLLRSDAFAVSTRWEV | TAF7L [263,282] |
| 6 | 835 | KRRGPAYVMELPKLKLSGVV | RAGE-1 [339,358] | 19 | 2228 | QTTFGPVFEDQPLSVLFPFE | TAG-1 [32,51] |
| 6 | 836 | IRKTQKIVIKKREPLNFGIP | RCAS1 [102,121] | 19 | 2229 | DYGNEEIIPLNRIYHLNRNI | TDRD1 |
| | | | | | | | [350,369] |
| 6 | 837 | PAPDNVLLTLRPRRINMTDT | SAGE1 [216,235] | 19 | 2230 | QTQLAKTLQEIFQAENTIML | TEKT5 [357,376] |
| 6 | 838 | APRLAEYQAYIDFEMKIGDP | SART3 [307,326] | 19 | 2231 | RITIGTLFSVLHERRSQFPV | TEX14 |
| | | | | | | | [328,347] |
| 6 | 839 | HDLEIQLTAITTSEQYYSKE | SCP-1 [479,498] | 19 | 2232 | REDNAVSAATALLESEEDTI | TEX15 |
| | | | | | | | [544,563] |
| 6 | 840 | RLKTIKQLYEQFIKSMEELE | SCP3a [171,190] | 19 | 2233 | SKPKAPATLLEENDPVPKPK | THEG [272,291] |
| 6 | 841 | NTREPAAEIRALLGMBLVRL | SCRN1 [96,115] | 19 | 2234 | FTKEVNEWMAQDLENIVATH | TPTE [318,337] |
| 6 | 842 | EAGAAALRNVAQRLFENYQT | se57-1 [121,140] | 19 | 2235 | ISMQNLEALLVANRDKEYQS | TSGA10 |
| | | | | | | | [562,581] |
| 6 | 843 | DFQKEYQLKTIEKLALEKSY | SLCO6A1 [132,151] | 19 | 2236 | VNITEYRASHSTPIEWYPDY | TSPY1 [222,241] |
| 6 | 844 | AAQLASMQVSPGAKMPSTPQ | SOX-6 [371,390] | 19 | 2237 | NFSFRNTLEGFASPLTGIAD | TYR [337,356] |
| 6 | 845 | SNTHYRIPQGFGNLLEGLTR | SP17 [6,25] | 19 | 2238 | GTDEAVLQVQAHEHGQEIFQ | ZNF165 |
| | | | | | | | [130,149] |
| 6 | 846 | TPNNHFTLEDIQALKRQYEL | SPAG1 [907,926] | 20 | 2239 | AALLAGRALQGLRRLELASN | 5T4 [201,220] |
| 6 | 847 | STAHSRIRKERPISLGIFPL | SPAG9 [190,209] | 20 | 2240 | ILSSLELWIDENKIATTGEA | ADAM2 |
| | | | | | | | [224,243] |
| 6 | 848 | HLSKSDLLANQSQEVLEERT | SPATA19 [93,112] | 20 | 2241 | RDFNQFSSGEKNIFLASFVH | AFP [337,356] |
| 6 | 849 | RSSWENIKFEDSVGLQMVSH | SPO11 [75,94] | 20 | 2242 | AREGGRFFPRERKRFRGQER | AKAP-3 |
| | | | | | | | [254,273] |
| 6 | 850 | GKHAWTHRLRERKQLVIYEE | SSX-1 [160,179] | 20 | 2243 | GGGQSAKALSVKQLESHRAP | AKAP-4 |
| | | | | | | | [588,607] |
| 6 | 851 | GFKAILPSFMRNKRVTDFQG | SSX-3 [62,81] | 20 | 2244 | IQNKPLYFADRLYDSMKGKG | ANXA2 |
| | | | | | | | [263,282] |
| 6 | 852 | ISFELRKAFDDIAKYFSKKE | SSX-4 [17,36] | 20 | 2245 | YLQKVVLKDLWKHSFSWPFQ | BRDT [34,53] |
| 6 | 853 | HRVRERKQLVIYEEISDPQE | SSX-5 [166,185] | 20 | 2246 | TEKPEFQSQVYNYAKDNNIK | CAGE1 |
| | | | | | | | [138,157] |
| 6 | 854 | MGQHKELWDFHMPERLAKEI | SYCE1 [166,185] | 20 | 2247 | QMVTDLELSTLLPISHENLT | CCDC62 |
| | | | | | | | [643,662] |
| 6 | 855 | DADSSAQAAAQAPENFQEGK | TAF7L [66,85] | 20 | 2248 | PKAKRTSRFLSGIINFIHFR | CDCA1 |
| | | | | | | | [112,131] |
| 6 | 856 | AAVALVSSSAWSSALGSQTT | TAG-1 [15,34] | 20 | 2249 | KLLEKFSLVQKEYEGYEVES | CTAGE2 [61,80] |
| 6 | 857 | IMLLKNFMLNQNVMLSVKGI | TDRD1 [1072,1091] | 20 | 2250 | RRVVAALPEGMRPDSNLYGF | CTAGE5 [20,39] |
| 6 | 858 | LVNEVFTIDDTLQTLKLRLR | TEKT5 [415,434] | 20 | 2251 | EQFTKIKELELMPEKGLKEE | CTCFL [11,30] |
| 6 | 859 | LLQISDALRYLHFQGFIHRS | TEX14 [357,376] | 20 | 2252 | PQVINAALTLAARPQSKVAQ | CTNNA2 |
| | | | | | | | [460,479] |
| 6 | 860 | KELRRHKIYGRKRRLTSQDS | TEX15 [933,952] | 20 | 2253 | VGSQAHVSFLDPRQPSYNVK | DCAF12 |
| | | | | | | | [313,332] |
| 6 | 861 | EDIPEISRLSISQKLPSTTM | THEG [97,116] | 20 | 2254 | GERPYWELSNHEVMKAINDG | EPHA2 |
| | | | | | | | [814,833] |
| 6 | 862 | ERRQQYFSELFNILDTAIIV | TPTE [148,167] | 20 | 2255 | LIGQGIIVKDARLNGSVASY | FAM46D |
| | | | | | | | [51,70] |
| 6 | 863 | SEIELLRSQMANERISMQNL | TSGA10 [548,567] | 20 | 2256 | ELDRLVWRNSIRSSFISSLS | FBXO39 |
| | | | | | | | [135,154] |
| 6 | 864 | SEVPVLQVIMHSRYRAQRFW | TSP50 [179,198] | 20 | 2257 | FLIMPLYGISVSLSSEDQHL | FSIP1 [537,556] |
| 6 | 865 | DRRGAVIQSVPGFWANVIAN | TSPY1 [146,165] | 20 | 2258 | LERGANASFEKDKQSILITA | GASZ [99,118] |
| 6 | 866 | EKDKFFAYLTLAKHTISSDY | TYR [130,149] | 20 | 2259 | YRIYEMENVLLGLFSTIHDS | Glypican-3 |
| | | | | | | | [311,330] |
| 6 | 867 | TGSQALLLRTPYSSDNLYQM | WT1 [210,229] | 20 | 2260 | HRRGSRSYVPLAHSSSAFTI | gp100 |
| | | | | | | | [190,209] |
| 6 | 868 | GKAFRHSSKLARHQRIHTGE | ZNF165 [350,369] | 20 | 2261 | MGFEPQIMKILLEVRPERQT | HAGE [404,423] |
| 7 | 869 | FSGSNASVSAPSPLVELILN | 5T4 [162,181] | 20 | 2262 | KPPLEKILPLLASLQRIRVR | JARID1B |
| | | | | | | | [1226,1245] |
| 7 | 870 | SQPVSILSPNTLKEIEASAE | ACRBP [124,143] | 20 | 2263 | DAKVRMVIITGPPEAQFKAQ | KOC1 [448,467] |
| 7 | 871 | IERGRILNWEGVQYLWSFVL | ACTL8 [67,86] | 20 | 2264 | RAKLQELNAIPPILDLLKSE | KU-CT-1 |
| | | | | | | | [184,203] |
| 7 | 872 | QDFAKYIEMHVIVEKQLYNH | ADAM2 [174,193] | 20 | 2265 | MIEEPRLYEKNKPFYKLQEV | LIPI [386,405] |
| 7 | 873 | SKHLPVFTYTDQGAILEDQP | ADAM29 [75,94] | 20 | 2266 | FPTVRPADLTRVIMPLEQRS | MAGE-A11 |
| | | | | | | | [98,117] |
| 7 | 874 | YKEVSKMVKDALTAIEKPTG | AFP [60,79] | 20 | 2267 | REKRRKAREETQGLKVAHAT | MAGE-B1 |
| | | | | | | | [12,31] |
| 7 | 875 | KQLQAVLQWVAASELNVPIL | AKAP-3 [771,790] | 20 | 2268 | WGPRAYAETSKMKVLEFLAK | MAGE-B2 |
| | | | | | | | [275,294] |
| 7 | 876 | KQLQAVLQWIAASQFNVPML | AKAP-4 [772,791] | 20 | 2269 | ATEEKIWEFLNKMRIYDGKK | MAGE-B3 |
| | | | | | | | [218,237] |
| 7 | 877 | PKWISIMTERSVPHLQKVET | ANXA2 [211,230] | 20 | 2270 | STSTERSLKDSLTRKTKMLV | MAGE-B4 |
| | | | | | | | [98,117] |
| 7 | 878 | LKAVHQQLQVLSQVPFRKLN | BRDT [430,449] | 20 | 2271 | EVIKRKVVEFLAMLKNTVPI | MAGE-C1 |
| | | | | | | | [1078,1097] |
| 7 | 879 | TMYRGNTTMDIKELVKQFHQ | CABYR [45,64] | 20 | 2272 | RKLQSIIYESNTRGIHNEIS | MORC1 |
| | | | | | | | [883,902] |
| 7 | 880 | EKVSDIMLQKLKSLHLKKKT | CAGE1 [645,664] | 20 | 2273 | LEVQIQHVVEGKRALSELTQ | MPHOSPH1 |
| | | | | | | | [1135,1154] |
| 7 | 881 | DSRFGHFRLSSGKFYGHKEK | CALR3 [43,62] | 20 | 2274 | RTGYTKTYQAHAKQKFSRLW | NLRP4 [92,111] |
| 7 | 882 | SRQMVITLELSTLLPISHEN | CCDC62 [641,660] | 20 | 2275 | KYFEDSRNMKTLKEPYLKGE | NXF2 [439,458] |
| 7 | 883 | KSSADKMQQLNAAHQEALMK | CDCA1 [146,165] | 20 | 2276 | AFEPAIEMQKSVPNKALELK | NY-BR-1 |
| | | | | | | | [704,723] |
| 7 | 884 | PASNMLKMEWSREVTTNAQR | CRISP2 [54,73] | 20 | 2277 | GLNVKRIIQELQKLMNKQHS | NYD-TSPG |
| | | | | | | | [573,592] |
| 7 | 885 | SQIDEFTGFSKERMMQKPGS | CT45 [71,90] | 20 | 2278 | ANVFGDGPYSTFLTSSPIRS | ODF2 [805,824] |
| 7 | 886 | KKASILLIRKIYILMQNLGP | CT46 [147,166] | 20 | 2279 | KLNWIPSFPTYDYFNQVTLQ | PASD1 [20,39] |
| 7 | 887 | DSGPDSSDVVPAAEVVGVAG | CT47 [37,56] | 20 | 2280 | LPRNMPSHRAQIMARYPEGY | PEPP2 |
| | | | | | | | [439,458] |
| 7 | 888 | LEGERNQIYIQLSEVEKTKE | CTAGE2 [299,318] | 20 | 2281 | HAFEGTILFLPKRLQQKVTE | PIWIL1 |
| | | | | | | | [161,180] |
| 7 | 889 | LLEKEPYALDVPNTAFGREH | CTAGE5 [497,516] | 20 | 2282 | IEGRVLPMERINLKNTSFIT | PIWIL2 |
| | | | | | | | [563,582] |
| 7 | 890 | MSGDERSDEIVLTVSNSNVE | CTCFL [206,225] | 20 | 2283 | KEYSIVKELAKHTRLSPRRR | PIWIL3 |
| | | | | | | | [413,432] |
| 7 | 891 | FGKEMVKLNYVAARRQQELK | CTNNA2 [179,198] | 20 | 2284 | ILKKLSMYQIGRNFYNPSEP | PIWIL4 |
| | | | | | | | [213,232] |
| 7 | 892 | GYVPQVDDIVNVVMVESIQF | CXorf48 [208,227] | 20 | 2285 | PLQALLERASATLQDLVFDE | PRAME |
| | | | | | | | [368,387] |
| 7 | 893 | SMVENKLVELEHTLLSKGFR | CXorf61 [85,104] | 20 | 2286 | DSVELAHYDVLLSYPNKTHP | PSMA [106,125] |
| 7 | 894 | VLGTVKWFNVRNGYGFINRN | DBPC [94,113] | 20 | 2287 | DERIAAHQALQHPYFQEQRK | RAGE-1 |
| | | | | | | | [271,290] |
| 7 | 895 | HKEWIFSIAWISDTMAVSGS | DCAF12 [183,202] | 20 | 2288 | QPQPIDNILSTASTGLINVAG | SAGE1 |
| | | | | | | | [262,281] |
| 7 | 896 | VAFWIIKLPRRRSHQDALEG | DKKL1 [167,186] | 20 | 2289 | EYENESQASQAVMKMDGMTI | SART3 |
| | | | | | | | [847,866] |
| 7 | 897 | SSQYRMHSYYPPPSYLGQSV | DMRT1 [285,304] | 20 | 2290 | PSSLTTPGSTLKFGAIRKMR | SCP-1 [933,952] |
| 7 | 898 | QQLGLSTNGKKIEVYLRLHR | DPPA2 [105,124] | 20 | 2291 | EKLEEKHSQITELENLVQRM | se57-1 |
| | | | | | | | [179,198] |
| 7 | 899 | VDEKAPEFSMQGLKAGVIAV | EpCAM [252,271] | 20 | 2292 | KDERRKILQAFPDMHNSNIS | SOX-6 [633,652] |
| 7 | 900 | KETFNLYYAESDLDYGTNFQ | EPHA2 [116,135] | 20 | 2293 | ETLEQYGKAYVDYKTVLQID | SPAG1 |
| | | | | | | | [532,551] |
| 7 | 901 | NLTWDQVITLDQVLDEVIPI | FAM46D [8,27] | 20 | 2294 | REAELKKEYNALHQRHTEMI | SPAG9 |
| | | | | | | | [148,167] |
| 7 | 902 | TRPKKMGSQLPKPRMLRESG | FALE1 [73,92] | 20 | 2295 | IYYTESQLFGNQTVVDNIIN | SPO11 |
| | | | | | | | [136,155] |
| 7 | 903 | RKWNQMMYSAELWRYRTITF | FBXO39 [44,63] | 20 | 2296 | DGTKEIESQGSIPGFLISSG | TAF7L [286,305] |
| 7 | 904 | RSLFWRSEPADDLQRQDNRV | FMR1NB [205,224] | 20 | 2297 | DIGETTIQLSWSRGFENHSP | TAG-1 |
| | | | | | | | [618,637] |
| 7 | 905 | AIQKMKKLEKILAKKQRREK | FSIP1 [118,137] | 20 | 2298 | VDVADKLVTFGLAKNITPQR | TDRD1 |
| | | | | | | | [1109,1128] |
| 7 | 906 | LSDEKMEHAQKLMRLQNLRG | FLHL17 [59,78] | 20 | 2299 | LYHREKRIGIDLVHDNVEKN | TEKT5 [194,213] |
| 7 | 907 | MAASALRGLPVAGGGESSES | GASZ [1,20] | 20 | 2300 | SVSTPLSPGSVSSAASQYKD | TEX14 |
| | | | | | | | [1209,1228] |
| 7 | 908 | GLYHKMNGQNRPLIKPKRRL | GATA-3 [289,308] | 20 | 2301 | HSNLLYSQYFTYFAGEPQAN | TEX15 |
| | | | | | | | [2705,2724] |
| 7 | 909 | VLLGLFSTIHESIQYVQKNA | Glypican-3 | 20 | 2302 | YVTERIIAMSETSSGRQSFY | TPTE [241,260] |
| | | | | | | | [319,338] |
| 7 | 910 | SQKRSFVYVWKTWGQYWQVL | gp100 [144,163] | 20 | 2303 | LAMKEKTISGMKNIIAEMEQ | TSGA10 |
| | | | | | | | [291,310] |
| 7 | 911 | PKASTWVVASRRSSTVSRAP | HAGE [8,27] | 20 | 2304 | EAYRRRHHNSSLNETNWFSD | TSPY1 [244,263] |
| 7 | 912 | TKYHSEDYIKFLRSIRPENM | HDAC1 [65,84] | 20 | 2305 | ASQSSMHNALHIYMNGTMSQ | TYR [357,376] |
| 7 | 913 | EMTKYHSDEYIKFLRSIRPD | HDAC2 [64,83] | 20 | 2306 | PGPALNVKLQPVETKAHETS | ZNF165 |
| | | | | | | | [155,174] |
| 7 | 914 | SEELPYSEYFEYFAPDFTLH | HDAC3 [320,339] | 21 | 2307 | VSLTYVSFRNLTHLESLHLE | 5T4 [247,266] |
| 7 | 915 | PSQKPSGFKSGQHPLNGQPL | HOM-TES-85 | 21 | 2308 | QSVEPQQDFAKYIEMHVIVE | ADAM2 |
| | | | [92,111] | | | | [168,187] |
| 7 | 916 | IALILGAFFYWRSKNKEEEE | IGFS11 [258,277] | 21 | 2309 | TKVNFTEIQKLVLDVAHVHE | AFP [248,267] |
| 7 | 917 | IREDDTTLVTATVENETYLL | IL13RA2 [276,295] | 21 | 2310 | KGTGSAEAVLQNAYQAIHNE | AKAP-3 |
| | | | | | | | [721,740] |
| 7 | 918 | ESYNEWALNVNEALEAKINK | JARID1B [753,772] | 21 | 2311 | GMKQANGQFIDKLVESVMKL | AKAP-4 |
| | | | | | | | [688,707] |
| 7 | 919 | QDTDTKITISPLQELTLYNP | KOC1 [304,323] | 21 | 2312 | KSYSPYDMLESIRKEVKGEL | ANXA2 |
| | | | | | | | [233,252] |
| 7 | 920 | VIDLMFHPGGLMKLRSREAD | KU-CT-1 [848,867] | 21 | 2313 | AQALEKLFMQKLSQMPQEEQ | BRDT [119,138] |
| 7 | 921 | REGAGRMRVVGWGLGSASPE | Lage-1 [142,161] | 21 | 2314 | QLKKELEKATASALDLLKRE | CAGE1 |
| | | | | | | | [457,476] |
| 7 | 922 | QSAYEIIKLKGYTSWAIGLS | LDHC [236,255] | 21 | 2315 | VGQLQAREQALTTMIKLKDK | CCDC62 |
| | | | | | | | [149,168] |
| 7 | 923 | LGFSPGPILPSTRKLYEKKL | LEMD1 [19,38] | 21 | 2316 | IQESLKTKIVESPEKLKNYK | CDCA1 |
| | | | | | | | [236,255] |
| 7 | 924 | DLFIPRIETILMMYTRNNLN | LIPI [56,75] | 21 | 2317 | KSLKSQVAEAKMTFKRFQAN | CTAGE2 |
| | | | | | | | [151,170] |
| 7 | 925 | VQAATSSSSPLVLGTLEEVP | MAGE-A1 [28,47] | 21 | 2318 | ISHEKKAHENWLAARNAERN | CTAGE5 |
| | | | | | | | [454,473] |
| 7 | 926 | KLLTQDWVQENYLEYRQVPG | MAGE-A10 | 21 | 2319 | SPQEMEVLQFHALEENVMVA | CLCFL [144,163] |
| | | | [269,288]; MAGE-A4 | | | | |
| | | | [245,264] | | | | |
| 7 | 927 | VKGLITKAEMLGSVIKNYED | MAGE-A11 | 21 | 2320 | TGIGELAAALNEFENKIILD | CTNNA2 |
| | | | [239,258] | | | | [271,290] |
| 7 | 928 | ETSFQVALSRKMAELVHFLL | MAGE-A12 | 21 | 2321 | VGTGQGSLLFYDIRAQRFLE | DCAF12 |
| | | | [102,121] | | | | [355,374] |
| 7 | 929 | FAHPRKLLMQDLVQENYLEY | MAGE-A2 [239,258] | 21 | 2322 | RLEGVISKYKPMMIITEYME | EPHA2 |
| | | | | | | | [677,696] |
| 7 | 930 | APEEKIWEELSVLEVFEGRE | MAGE-A3 [216,235]; | 21 | 2323 | NNRLKSLSIQYLELDRLVWR | FBXO39 |
| | | | MAGE-A6 [216,235] | | | | [123,142] |
| 7 | 931 | PVIFGKASESLKMIFGIDVK | MAGE-A4 [149,168] | 21 | 2324 | EASKGYYLTKALTGHNMSEA | FSIP1 [477,496] |
| 7 | 932 | GGPRISYPLLHEWALREGEE | MAGE-A6 [295,314] | 21 | 2325 | VSLVQELLDSGISVDSNFQY | GASZ [59,78] |
| 7 | 933 | LRKLLTQEWVQENYLEYRQA | MAGE-A8 [245,264] | 21 | 2326 | TLSSRRRELIQKLKSFISFY | Glypican-3 |
| | | | | | | | [383,402] |
| 7 | 934 | KLKVAELVHFLLHKYRYKEP | MAGE-A9 [109,128] | 21 | 2327 | GVSRQLRTKAWNRQLYPEWT | gp100 [34,53] |
| 7 | 935 | EERAQVRSSVRARRRTTATT | MAGE-B1 [312,331] | 21 | 2328 | PPIKKNFYKESTATSAMSKV | HAGE [192,211] |
| 7 | 936 | QRAPTTAAAAAAGVSSTKSK | MAGE-B2 [63,82] | 21 | 2329 | WEEFADPFAFIHKIRPIAEQ | JARID1B [39,58] |
| 7 | 937 | IEKKQSFSQGLSSTVQSRTD | MAGE-B3 [90,109] | 21 | 2330 | GKTVNELQNLSSAEVVVPRD | KOC1 [507,526] |
| 7 | 938 | NQSSAWTLPRNGLLMPLLSV | MAGE-B4 [188,207] | 21 | 2331 | YSQTGYLSSSNIINDGFYDY | KU-CT-1 |
| | | | | | | | [556,575] |
| 7 | 939 | IYDGILHSIYGDARKIITED | MAGE-B6 [316,335] | 21 | 2332 | LSVHIKNLLKHGASLDNEHE | LIPI [157,176] |
| 7 | 940 | SLLQIPMTSSFSSTLLSIFQ | MAGE-C1 [336,355] | 21 | 2333 | AQALQAEEQEAAFFSSTLNV | MAGE-A11 |
| | | | | | | | [138,157] |
| 7 | 941 | MIVIKYKDYFPVILKRAREF | MAGE-C2 [169,188] | 21 | 2334 | LKGNSATEEEIWKFMNVLGA | MAGE-B1 |
| | | | | | | | [210,229] |
| 7 | 942 | EEESLSEVVVPMPSWLIRTR | MORC1 [135,154] | 21 | 2335 | LITKELVQEKYLEYKQVPSS | MAGE-B2 |
| | | | | | | | [247,266] |
| 7 | 943 | QKFGDFLQHSPSILQSKAKK | MPHOSPH1 | 21 | 2336 | FPEILKKASFNMEVVEGVEL | MAGE-B3 |
| | | | [1731,1750] | | | | [149,168] |
| 7 | 944 | TSASGSASGSASTLVHNGTS | MUC-1 [959,978] | 21 | 2337 | DPPRYQFLWGPRAHAETSKM | MAGE-B4 |
| | | | | | | | [264,283] |
| 7 | 945 | PTPSKPLTSFLIQDILREGA | NKX3.1 [20,39] | 21 | 2338 | DELARELLLKYQVKQPITKA | MAGE-C1 |
| | | | | | | | [913,932] |
| 7 | 946 | MMAWSDNKIERDRFLYTFYF | NLRP4 [169,188] | 21 | 2339 | PEEASDIIYFGRSKKRLSTL | MORC1 [74,93] |
| 7 | 947 | RLADELLFRQIAWIKKLPFF | NR6A1 [289,308] | 21 | 2340 | DSDDTLYGSLTNSLNISEFE | MPHOSPH1 |
| | | | | | | | [231,250] |
| 7 | 948 | FPKLLRLDGRELSAPVIVDI | NXF2 [350,369] | 21 | 2341 | MEKRPVQVLLSSLLRKKMLP | NLRP4 |
| | | | | | | | [252,271] |
| 7 | 949 | PKKPSAFKPAIEMQNSVPNK | NY-BR-1 [461,480] | 21 | 2342 | KWHSEDEIRITTWRNRKPPE | NXF2 [90,109] |
| 7 | 950 | PNDYTKEVAEYEQERQMLGT | NYD-TSPG [185,204] | 21 | 2343 | QIVEFLLIKNANANAVNKYK | NY-BR-1 |
| | | | | | | | [130,149] |
| 7 | 951 | KLVEAEMDGAAAAKQVMALK | ODF2 [210,229] | 21 | 2344 | TEKFDLSNLQNNYAHLTNSS | NYD-TSPG |
| | | | | | | | [282,301] |
| 7 | 952 | MGPNTVGKASQPSFSIKGRS | ODF3 [148,167] | 21 | 2345 | VAEALSTLESWRSRYNQVVK | ODF2 [434,453] |
| 7 | 953 | AFILLLVVAFSKKWLDLSRS | ODF4 [91,110] | 21 | 2346 | RLWQELSDSLGPVVQVNTWS | PASD1 |
| | | | | | | | [691,710] |
| 7 | 954 | LKEKIVLTHNRLKSLMKILS | OIP5 [199,218] | 21 | 2347 | PKTMVNISDQTMHSIPTSPS | PEPP2 |
| | | | | | | | [527,546] |
| 7 | 955 | EQLEERTWLLHDAIQNQQNA | PASD1 [382,401] | 21 | 2348 | YHDMTAGRRSIAGEVASINE | PIWIL1 |
| | | | | | | | [634,653] |
| 7 | 956 | NMARGLKYLHQEKKLLHGDI | PBK [149,168] | 21 | 2349 | KTPKDSFTMSDGKEITFLEY | PIWIL2 |
| | | | | | | | [436,455] |
| 7 | 957 | DLNLEWISLPRSWTYGITRG | PEPP2 [4,23] | 21 | 2350 | LLQLWELKFDTNFLSVPGRV | PIWIL3 |
| | | | | | | | [452,471] |
| 7 | 958 | YNPLMEARRLRSALLFQHED | PIWIL1 [136,155] | 21 | 2351 | ETQRGETIKMTITLKRELPS | PIWIL4 |
| | | | | | | | [178,197] |
| 7 | 959 | LVGNIVITRYNNRTYRIDDV | PIWIL2 [413,432] | 21 | 2352 | MLTDVSPEPLQALLERASAT | PRAME |
| | | | | | | | [360,379] |
| 7 | 960 | GSEGTVVQLLANHFRVISRP | PIWIL3 [119,138] | 21 | 2353 | TLRGAVEPDRYVILGGHRDS | PSMA [361,380] |
| 7 | 961 | QYAHKLTFLVAQSIHKEPSL | PIWIL4 [824,843] | 21 | 2354 | LQSVLGSGTNGRVPVLRPLK | RAGE-1 |
| | | | | | | | [368,387] |
| 7 | 962 | ETFKAVLDGLDVLLAQEVRP | PRAME [95,114] | 21 | 2355 | RSTRDLYATVIHDIQEEEME | SAGE1 |
| | | | | | | | [710,729] |
| 7 | 963 | FEGRTRYSRITGGMEAEVGE | PRSS55 [59,78] | 21 | 2356 | DGMTIKENIIKVAISNPPQR | SART3 |
| | | | | | | | [862,881] |
| 7 | 964 | KYADKIYSISMKHPQEMKTY | PSMA [606,625] | 21 | 2357 | DSSETTELLSMVSEEETLKT | SCP-1 [893,912] |
| 7 | 965 | YDPDERIAAHQALQHPYFQE | RAGE-1 [268,287] | 21 | 2358 | KLSLENKLLQLKSSATYGKS | se57-1 |
| | | | | | | | [209,228] |
| 7 | 966 | ISSTITRDLYVTATHSVHEE | SAGE1 [660,679] | 21 | 2359 | NEKERTRFENLGPQLTGKSN | SOX-6 [539,558] |
| 7 | 967 | EKVHSLERRQLAIPLYDMEA | SART3 [245,264] | 21 | 2360 | PASVPLQAWHPAKEMISKQA | SPAG1 |
| | | | | | | | [587,606] |
| 7 | 968 | EQEQSSLRASLEIELSNLKA | SCP-1 [739,758] | 21 | 2361 | SIRLDSTLRLYHAHTYQHLQ | SPAG9 |
| | | | | | | | [1105,1124] |
| 7 | 969 | NLLTGAQNEFKKEMAMLQKK | SCP3a [195,214] | 21 | 2362 | KGVPDLNTRLLVKKLWDTFH | SPO11 |
| | | | | | | | [250,269] |
| 7 | 970 | DQEQGRKLRSTMLELEKQGL | SCRN1 [361,380] | 21 | 2363 | PDVKEMEKSSFTEYIESPDV | TAF7L [242,261] |
| 7 | 971 | ERKLSLENKLLQLKSSATYG | se57-1 [207,226] | 21 | 2364 | VESKFAQLNLAAEDTRLFAP | TAG-1 |
| | | | | | | | [220,239] |
| 7 | 972 | DRKKVIWFVASSFLIGLGSL | SLCO6A1 [167,186] | 21 | 2365 | ESVLVGYVDYGNFEILSLMR | TDRD1 |
| | | | | | | | [570,589] |
| 7 | 973 | EDAEGSKAMNGSAAKLQQYY | SOX-6 [574,593] | 21 | 2366 | IDDTLQTLKLRLRETQDTLQ | TEKT5 [422,441] |
| 7 | 974 | SIPFSNTHYRIPQGFGNLLE | SP17 [2,21] | 21 | 2367 | PEHSEAFQASSETLVAVEKS | TEX14 |
| | | | | | | | [1042,1061] |
| 7 | 975 | EPAGSEIADDLSILYSNRAA | SPAG1 [476,495] | 21 | 2368 | NPETDVSLVPDASVLSKPIF | TEX15 |
| | | | | | | | [2497,2516] |
| 7 | 976 | DEEVVKELMPLVVAVLENLD | SPAG9 [43,62] | 21 | 2369 | ILADLIFTDSKLYIPLEYRS | TPTE [109,128] |
| 7 | 977 | EMTEDIMRDRIEQVRRSISR | SPATA19 [130,149] | 21 | 2370 | VANRDKEYQSQIALQEKESE | TSGA10 |
| | | | | | | | [572,591] |
| 7 | 978 | PTGGSRLASSSEVLASIENI | SPO11 [35,54] | 21 | 2371 | SANDPIFLLHHAFVDSIFEQ | TYR [380,399] |
| 7 | 979 | EKRSKAFDDIATYFSKKEWK | SSX-1 [19,38] | 22 | 2372 | NRNLTEVPTELPAYVRNLFL | 5T4 [79,98] |
| 7 | 980 | MNGDDAFARRPTVGAQIPEK | SSX-2 [1,20] | 22 | 2373 | LVLRPHDVAFLLVYREKSNY | ADAM2 |
| | | | | | | | [258,277] |
| 7 | 981 | MNGDDTFARRPTVGAQIPEK | SSX-3 [1,20] | 22 | 2374 | FQKLGEYYLQNAFLVAYTKK | AFP [419,438] |
| 7 | 982 | VTLPPFMRSKRAADFHGNDF | SSX-4 [65,84] | 22 | 2375 | SDSWAEDLIVSALLLIQYHL | AKAP-3 |
| | | | | | | | [520,539] |
| 7 | 983 | KYFSEKEWEKMKASEKIIYV | SSX-5 [30,49] | 22 | 2376 | MVYANQVASEMMVSLMKTLK | AKAP-4 |
| | | | | | | | [334,353] |
| 7 | 984 | VHLKEILSKKQETLRILRLH | SYCE1 [96,115] | 22 | 2377 | RIMVSRSEVDMLKIRSEFKR | ANXA2 |
| | | | | | | | [290,309] |
| 7 | 985 | NEGTSSIVMEIQKQIEKKEK | TAF7L [384,403] | 22 | 2378 | DVFETHFSKIPIEPVESMPL | BRDT [367,386] |
| 7 | 986 | VSREAILRFGFLQEFSKEER | TAG-1 [121,140] | 22 | 2379 | QVFIDVINKLKENVEELIED | CAGE1 |
| | | | | | | | [348,367] |
| 7 | 987 | LGVTKEIAIWAERIMFSDLR | TDRD1 [237,256] | 22 | 2380 | IYVKQQSDLQFLNFNVENSQ | CCDC62 |
| | | | | | | | [288,307] |
| 7 | 988 | TILPTLRSALFSRYSPHDWD | TEKT5 [76,95] | 22 | 2381 | KMKETVQKLKNARQEVVEKY | CDCA1 |
| | | | | | | | [257,276] |
| 7 | 989 | PPSLNYIPPVLQLSGGQKPD | TEX14 [796,815] | 22 | 2382 | ILHEKKAHDNWSAAWTAERN | CTAGE2 |
| | | | | | | | [423,442] |
| 7 | 990 | YGLEHIFFDAAKNLVWKERT | TEX15 [1882,1901] | 22 | 2383 | LTETELKFELLEKBPYALDV | CTAGE5 |
| | | | | | | | [488,507] |
| 7 | 991 | RPKRFYLEYYNNNRTTPVWP | THEG [199,218] | 22 | 2384 | KYASVEASKLKRHVRSHTGE | CTCFL [348,367] |
| 7 | 992 | IALFFLMDVLLRVFVERRQQ | TPTE [133,152] | 22 | 2385 | SNNEEGVKLVRMAATQIDSL | CTNNA2 |
| | | | | | | | [439,458] |
| 7 | 993 | RRQLDETNDELAQIARERDI | TSGA10 [330,349] | 22 | 2386 | RHNVSRVPVYAHITHKALKD | DCAF12 |
| | | | | | | | [223,242] |
| 7 | 994 | KSEAPPIYLQVSSYQHWIWD | TSP50 [336,355] | 22 | 2387 | QSPEDVYFSKSEQLKPLKTY | EPHA2 |
| | | | | | | | [569,588] |
| 7 | 995 | HHNSSLNFFNWFSDHNFAGS | TSPY1 [250,269] | 22 | 2388 | QFKKTMSTFHNLVSLNLNYN | FBXO39 |
| | | | | | | | [215,234] |
| 7 | 996 | KHTISSDYVIPIGTYGQMKN | TYR [142,161] | 22 | 2389 | LREIDEKLKMMKENVLESTS | FSIP1 [382,401] |
| 7 | 997 | IQDVRRVPGVAPTLVRSASE | WT1 [297,316] | 22 | 2390 | LITAVQNVITELPVNSQKIT | GASZ [370,389] |
| 7 | 998 | FKWNSDFINHQIIYAGEKNH | ZNF165 [301,320] | 22 | 2391 | IHDSIQYVQKNAGKLTTTIG | Glypican-3 |
| | | | | | | | [327,346] |
| 8 | 999 | LRHLDLSNNSLVSLTYVSFR | 5T4 [236,255] | 22 | 2392 | SGYLAEADLSYTWBFGESSG | gp100 |
| | | | | | | | [248,267] |
| 8 | 1000 | PWPERLSNNVEELLQSSLSL | ACRBP [167,186] | 22 | 2393 | DLASRGLDVHDVTHVYNFDF | HAGE [545,564] |
| 8 | 1001 | LYPGFTKRLFRELMGDHVSS | ACTL8 [303,322] | 22 | 2394 | DQIAKYWELQGSTLKIPHVE | JARID1B |
| | | | | | | | [105,124] |
| 8 | 1002 | IFVSFNITIILSSLELWIDE | ADAM2 [215,234] | 22 | 2395 | LQELTLYNPERTITVKGNVE | KOC1 [315,334] |
| 8 | 1003 | QKQSSYVPWWIHFRIVEIVV | ADAM29 [185,204] | 22 | 2396 | FSWELHISELKFQLKSNVIP | KU-CT-1 |
| | | | | | | | [767,786] |
| 8 | 1004 | KLSQKFTKVNFTEIQKLVLD | AFP [242,261] | 22 | 2397 | KNKPFYKLQEVKILAQFYND | LIPI [395,414] |
| 8 | 1005 | DAMLRKLYNVMFAKKVPEHV | AKAP-3 [375,394] | 22 | 2398 | SSVRARRRTTATTFRARSRA | MAGE-B1 |
| | | | | | | | [319,338] |
| 8 | 1006 | MTDSDFVSAVKRNLFNQWKQ | AKAP-4 [398,417] | 22 | 2399 | TTAAAAAAGVSSTKSKKGAK | MAGE-B2 |
| | | | | | | | [67,86] |
| 8 | 1007 | RYKSYSPYDMLESIRKEVKG | ANXA2 [231,250] | 22 | 2400 | GRSRSALKKPQRALSTTTSV | MAGE-B3 |
| | | | | | | | [56,75] |
| 8 | 1008 | PSRQTAIIVNPPPPEYINTK | BRDT [4,23] | 22 | 2401 | EEERAGARPRVAARRGTTAM | MAGE-B4 |
| | | | | | | | [311,330] |
| 8 | 1009 | EVPAQLLDAEGAIKIGSEKS | CABYR [397,416] | 22 | 2402 | FPSSTSSSLSQSSPVSSFPS | MAGE-C1 |
| | | | | | | | [817,836] |
| 8 | 1010 | ADQRLAISHSQIAHLEERNK | CAGE1 [715,734] | 22 | 2403 | VVPMPSWLIRTRESVTDDPQ | MORC1 |
| | | | | | | | [143,162] |
| 8 | 1011 | TQYDLSEFENIGAIGLELWQ | CALR3 [287,306] | 22 | 2404 | QDVKGYSFIKELQWIQVSDS | MPHOSPH1 |
| | | | | | | | [304,323] |
| 8 | 1012 | VEWMSIFKPSKMQRIVRLKS | CCDC62 [532,551] | 22 | 2405 | AAPITEIVSQPERLLFVIDS | NLRP4 |
| | | | | | | | [213,232] |
| 8 | 1013 | EKTKRLNELKLSVVSLKEIQ | CDCA1 [218,237] | 22 | 2406 | NKLKPGQMEMLKLTMNKRYN | NXF2 [207,226] |
| 8 | 1014 | ALLARKQGAGDSLIAGSAMS | CT45 [34,53] | 22 | 2407 | ASLTPLLLSITKRSEQIVEF | NY-BR-1 |
| | | | | | | | [115,134] |
| 8 | 1015 | ESPELSISHSQVEQLVNKTS | CT46 [306,325] | 22 | 2408 | NNYAHLTNSSINKSGASYEK | NYD-TSPG |
| | | | | | | | [292,311] |
| 8 | 1016 | LKEFEKTIHFYQKKIILHEK | CTAGE2 [408,427] | 22 | 2409 | LKRLAEADSEKARLLLLLQD | ODF2 [294,313] |
| 8 | 1017 | TLEGERNQIYIQLSEVEKTK | CTAGE5 [328,347] | 22 | 2410 | LVQQEQHLKEQQRQLREQLQ | Pasd1 |
| | | | | | | | [482,501] |
| 8 | 1018 | TLQTVHFTSEAVELQEMSLL | CTCFL [81,100] | 22 | 2411 | SNSIKRNPNAPVVRRGWLYK | PEPP2 |
| | | | | | | | [159,178] |
| 8 | 1019 | LVYDGVRDIRKAVLMIRTPE | CTNNA2 [615,634] | 22 | 2412 | MEARRLRSALLFQHEDLIGK | PIWIL1 |
| | | | | | | | [140,159] |
| 8 | 1020 | QQGLPQGDTQLTTVQGVVTS | CXorf48 [26,45] | 22 | 2413 | TIGQPTRLRSVAQKILLQIN | PIWIL2 |
| | | | | | | | [703,722] |
| 8 | 1021 | TENNLAVYDLSRDILNNETH | CXorf61 [53,72] | 22 | 2414 | VGQSIHQEPNRSLSTRLFYL | PIWIL3 |
| | | | | | | | [863,882] |
| 8 | 1022 | VPPPRFRPRYRRPFRPRPRQ | DBPC [272,291] | 22 | 2415 | GLITQATSDFQLMKAVAEKT | PIWIL4 |
| | | | | | | | [384,403] |
| 8 | 1023 | ERGSGIRSVSFYEHIITVGT | DCAF12 [338,357] | 22 | 2416 | NSHQDFWTVWSGNRASLYSF | PRAME |
| | | | | | | | [127,146] |
| 8 | 1024 | PRMEEKEALVPIQKATDSFH | DXKL1 [141,160] | 22 | 2417 | SDIVPPFSAFSPQGMPEGEL | PSMA [155,174] |
| 8 | 1025 | ASEGRMVIQDIPAVTSRGHV | DMRT1 [178,197] | 22 | 2418 | LMKEVRRFGQNYERIFILLE | SAGE1 |
| | | | | | | | [831,850] |
| 8 | 1026 | KIEVYLRLHRHAYPEQRQDM | DRRA2 [115,134] | 22 | 2419 | FIQATDYVEIWQAYLDYLRR | SART3 |
| | | | | | | | [411,430] |
| 8 | 1027 | TVNGEQLDLDPGQTLIYYVD | EpCAM [234,253] | 22 | 2420 | LSRVYSKLYKEAEKIKKWKV | SCP-1 [102,121] |
| 8 | 1028 | DLEPHMNYTFTVEARNGVSG | ERHA2 [401,420] | 22 | 2421 | ESTSAHIIEETEYVKKIRTT | se57-1 [24,43] |
| 8 | 1029 | AKLTKESYPVVVAESMYGDF | FAM46D [192,211] | 22 | 2422 | TQVKDEAAAQPLNLSSRPKT | SOX-6 [414,433] |
| 8 | 1030 | LQEYAGNFQGIRFHYDRNPG | FATE1 [98,117] | 22 | 2423 | EDYKEKTVIDKSHLSKIETR | SPAG1 |
| | | | | | | | [175,194] |
| 8 | 1031 | QILDSLSYMRNENVISELNI | FBXO39 [181,200] | 22 | 2424 | QRHTEMIHNYMEHLERTKLH | SPAG9 |
| | | | | | | | [161,180] |
| 8 | 1032 | ESLKMRVSKPFGMLMLSIWI | FMR1NB [59,78] | 22 | 2425 | MIYKLVQSNTYATKRDIYYT | SPO11 |
| | | | | | | | [120,139] |
| 8 | 1033 | ILKQKSIIKLSSERKKEDIE | FSIP1 [416,435] | 22 | 2426 | EYLERQLQAEFIESGQYRAN | TAF7L [365,384] |
| 8 | 1034 | HEQVKTIKELGGYVSNLRKI | FTHL17 [140,159] | 22 | 2427 | QNRVEVLAGELRFSKLSLED | TAG-1 |
| | | | | | | | [370,389] |
| 8 | 1035 | DENGYTALTWAARQGHKNIV | GASZ [179,198] | 22 | 2428 | MFSDLRSLQLKKTMEIKGTV | TDRD1 |
| | | | | | | | [251,270] |
| 8 | 1036 | KLESSHSRGSMTALGGASSS | GATA-3 [195,214] | 22 | 2429 | ERRSQFPVLHMEVIVHLLLQ | TEX14 |
| | | | | | | | [340,359] |
| 8 | 1037 | YYPEDLFIDKKVLKVAHVEH | Glypican-3 | 22 | 2430 | ELDHLALEWQITPSFESLSQ | TEX15 |
| | | | [361,380] | | | | [320,339] |
| 8 | 1038 | SYTWDFGESSGTLISRALVV | gp100 [257,276] | 22 | 2431 | RLLRLIILLRIFHLFHQKRQ | TPTE [195,214] |
| 8 | 1039 | KALENFKTGKVRILIATELA | HAGE [528,547] | 22 | 2432 | LAENKMAIQSRDVAQFRNVV | TSGA10 |
| | | | | | | | [601,620] |
| 8 | 1040 | QRLFENLRMLPHAPGVQMQA | HDAC1 [364,383]; | 22 | 2433 | RNTLEGFASPLTGIADASQS | TYR [341,360] |
| | | | HDAC2 [365,384] | | | | |
| 8 | 1041 | YGLYKKMIVFKPYQASQHDM | HDAC3 [39,58] | 23 | 2434 | LAQLPSLRHLDLSNNSLVSL | 5T4 [230,249] |
| 8 | 1042 | NHHSERSRNHLERSLSQSDR | HOM-TES-85 | 23 | 2435 | VIEGKPYTVNLMQKNFLPHN | ADAM2 [50,69] |
| | | | | | | | [156,175] |
| 8 | 1043 | RKPRPPHTHSYTISHATLER | IGFS11 [394,413] | 23 | 2436 | YLQNAFLVAYTKKAPQLTSS | AFP [426,445] |
| 8 | 1044 | TNGSEVQSSWAETTYWISPQ | IL13RA2 [114,133] | 23 | 2437 | ERPLASSPPRLYEDDETPGA | AKAP-3 |
| | | | | | | | [630,649] |
| 8 | 1045 | GEARLREMEALQSLRLANEG | JARID1B | 23 | 2438 | LIMAKYSNEGAALAELEEQA | AKAP-4 |
| | | | [1146,1165] | | | | [709,728] |
| 8 | 1046 | QVNTESETAVVNVTYSSKDQ | KOC1 [112,131] | 23 | 2439 | DTTTPATSAVKASSEFSPTF | BRDT [216,235] |
| 8 | 1047 | THEDKIVRRNATMIEGILAS | KU-CT-1 [77,96] | 23 | 2440 | KFHWVEAFDDEMTEKPEFQS | CAGE1 |
| | | | | | | | [126,145] |
| 8 | 1048 | VLKEFTVSGIILLEMSVREQD | Lage-1 [122,141] | 23 | 2441 | STIEKQRKELQLLIGELKDR | CCDC62 [21,40] |
| 8 | 1049 | GETRLALVQRNVAIMKSIIP | LDHC [103,122] | 23 | 2442 | SNSGRLSGPAELRSFNMPSL | CTAGE5 |
| | | | | | | | [629,648] |
| 8 | 1050 | LEGAQDSDESEELNIILQGN | LEMD1 [58,77] | 23 | 2443 | ATIIARKSDLRVHMRNLHAY | CTCFL [434,453] |
| 8 | 1051 | DGSFSFKLLITQLGMIEEPRL | LIPI [373,392] | 23 | 2444 | FIDASRLVYDGVRDIRKAVL | CTNNA2 |
| | | | | | | | [609,628] |
| 8 | 1052 | APEEEIWEELSVMEVYDGRE | MAGE-A1 [209,228] | 23 | 2445 | YGSEWSVYAVGSQAHVSFLD | DCAF12 |
| | | | | | | | [304,323] |
| 8 | 1053 | DPARYEFLWGPRAHAEIRKM | MAGE-A10 | 23 | 2446 | IEKVVQMTNEDIKRIGVRLP | EPHA2 |
| | | | [290,309] | | | | [933,952] |
| 8 | 1054 | REDSGEFGLQVSTMESEDDF | MAGE-A11 [23,42] | 23 | 2447 | MYSAELWRYRTITFSGRPSR | FBXO39 [50,69] |
| 8 | 1055 | EEQETASSSSTLVEVTLREV | MAGE-A12 [34,53] | 23 | 2448 | DVERNESLIKSGKKPFSNTE | FSIP1 [223,242] |
| 8 | 1056 | EEQQTASSSSTLVEVTLGEV | MAGE-A2 [34,53] | 23 | 2449 | FTKNGITSKDQQKILAALKE | GASZ [313,332] |
| 8 | 1057 | SEFQAALSRKVAELVHFLLL | MAGE-A3 [103,122] | 23 | 2450 | LGSDINVDDMVNELFDSLFP | Glypican-3 |
| | | | | | | | [157,176] |
| 8 | 1058 | DGREHTVYGEPRKLLTQDWV | MAGE-A4 [233,252] | 23 | 2451 | ANASFSIALNFPGSQKVLPD | gp100 [80,99] |
| 8 | 1059 | GSDPVRYEFLWGPRALAETS | MAGE-A8 [266,285] | 23 | 2452 | QKPTPIQSQAWPIVLQGIDL | HAGE [263,282] |
| 8 | 1060 | ETSYEKVIWYLVMLNAREPI | MAGE-A9 [279,298] | 23 | 2453 | VPTELVEKEFWRINSTIEEE | JARID1B |
| | | | | | | | [401,420] |
| 8 | 1061 | PRAYAETTKMKVLEFLAKMN | MAGE-B1 [274,293] | 23 | 2454 | IDVHRKENAGAAEKSITILS | KOC1 [229,248] |
| 8 | 1062 | KASEGLSVVFGLELNKVNPN | MAGE-B2 [155,174] | 23 | 2455 | EENKTTLLELGAVEPLTKLL | KU-CT-1 [57,76] |
| 8 | 1063 | FWAKVNKTVPSAFQFWYEEA | MAGE-B3 [291,310] | 23 | 2456 | GLEPAGPRFSRKPPYSRLDY | LIPI [202,221] |
| 8 | 1064 | DTTPNNFPLLYEEALRDEEE | MAGE-B4 [294,313] | 23 | 2457 | FSQGLSSTVQSRTDPLIMKT | MAGE-B3 |
| | | | | | | | [96,115] |
| 8 | 1065 | PHLYEBALTBEVERALRLRA | MAGE-B6 [388,407] | 23 | 2458 | VLRETASSSLAFGIPQEPQR | MAGE-B4 |
| | | | | | | | [45,64] |
| 8 | 1066 | IESEPLETYTLDEKVDELAR | MAGE-C1 [898,917] | 23 | 2459 | RELLTKVWVQEHYLEYREVP | MAGE-C1 |
| | | | | | | | [1041,1060] |
| 8 | 1067 | KKKVLEFLAKLNNTVPSSFP | MAGE-C2 [311,330] | 23 | 2460 | KISEDKLKNLRIKLALLLQK | MORC1 |
| | | | | | | | [911,930] |
| 8 | 1068 | EKQRELKTARTLSLFYGVNV | MORC1 [341,360] | 23 | 2461 | KMKESDHQIIKRRLRTKTAK | MPHOSPH1 |
| | | | | | | | [1801,1820] |
| 8 | 1069 | KKIIETMSSSKLSNVEASKE | MPHOSPH1 | 23 | 2462 | QSTTSVYSSFVFNLFTPEGA | NLRP4 |
| | | | [1749,1768] | | | | [366,385] |
| 8 | 1070 | AASRYNLTISDVSVSEVPFP | MUC-1 [1128,1147] | 23 | 2463 | NVSQQALDLQNLRFDPDLMG | NXF2 [226,245] |
| 8 | 1071 | EAFSRASLVSVYNSYPYYPY | NKX3.1 [203,222] | 23 | 2464 | NVDVSSTIYNNEVLHQPLSE | NY-BR-1 |
| | | | | | | | [1180,1199] |
| 8 | 1072 | KFKEHLKQMTLQLELKQIPW | NLPP4 [25,44] | 23 | 2465 | FELFGFDILIDDNLKPWLLE | NYD-TSPG |
| | | | | | | | [367,386] |
| 8 | 1073 | LLSSTWQELILLSSLTVYSK | NR6A1 [319,338] | 23 | 2466 | EKAQAKTASELSKSMESMRG | ODF2 [328,347] |
| 8 | 1074 | SETLKHLVLQFLQQYYSIYID | NXF2 [385,404] | 23 | 2467 | VAFNQQQLVQQEQHLKEQQR | PASD1 |
| | | | | | | | [475,494] |
| 8 | 1075 | AQRKSKSLKINLNYAGBALR | NY-BR-1 | 23 | 2468 | RSMRDDTMWQLYEWQQRQFY | PEPP2 |
| | | | [1200,1219] | | | | [493,512] |
| 8 | 1076 | INKRKLVHDIIDLIYLNGLR | NYD-TSPG [402,421] | 23 | 2469 | ADGSEVSFLEYYRKQYNQEI | PIWIL1 |
| | | | | | | | [335,354] |
| 8 | 1077 | SMQNYVQFLKSSYANVEGDG | ODF2 [792,811] | 23 | 2470 | RGPAQRESVGLVSMFRGLGI | PIWIL2 [61,80] |
| 8 | 1078 | SISARTKAFRVESTPGPAAY | ODF3 [123,142] | 23 | 2471 | RNEWYDFFIVSQSVQDGTVT | PIWIL3 |
| | | | | | | | [792,811] |
| 8 | 1079 | DLSRSLFYQRWPVDVSNRIH | ODF4 [106,125] | 23 | 2472 | NPAAFVRAIQQYVDPDVQLV | PIWIL4 |
| | | | | | | | [528,547] |
| 8 | 1080 | SMEWDTQVVKGSSPLGPAGL | OIP5 [36,55] | 23 | 2473 | SLEFLRGRLDQLLRHVMNPL | PRAME |
| | | | | | | | [305,324] |
| 8 | 1081 | SQGQRGHTSMKAVYVEPAAA | PASD1 [212,231] | 23 | 2474 | KLGSGNEFEVFFQRLGIASG | PSMA [514,533] |
| 8 | 1082 | MQKLGEGTGVNVYLMKRSPR | PBK [35,54] | 23 | 2475 | YKPDSNEFAVGTKNYSVSAG | SAGE1 |
| | | | | | | | [777,796] |
| 8 | 1083 | LHKEKYTLEQALLSASQEIE | PEPP2 [659,678] | 23 | 2476 | LQQLEKYKPYEEALLQAEAP | SART3 |
| | | | | | | | [289,308] |
| 8 | 1084 | VGQSIHREPNLSLSNRLYYL | PIWIL1 [842,861] | 23 | 2477 | TPEIYWKLDSKAVPSQTVSR | SCP-1 [803,822] |
| 8 | 1085 | HFWALFYPKRAMDQARELVN | PIWIL2 [604,623] | 23 | 2478 | VEGKKLRIGEYKQLMRSRRQ | SOX-6 [700,719] |
| 8 | 1086 | SREIRELPLLNAMPLHSWLI | PIWIL3 [493,512] | 23 | 2479 | LPTVVAYNNRAQAEIKLQNW | SPAG1 |
| | | | | | | | [239,258] |
| 8 | 1087 | QHKLSLWPGFAISVSYFERK | PIWIL4 [237,256] | 23 | 2480 | LMELQEAVRWTEMIRASREN | SPAG9 |
| | | | | | | | [522,541] |
| 8 | 1088 | LQALYVDSLFFLRGRLDQLL | PRAME [298,317] | 23 | 2481 | LLPSDLKRLNVPKDSLIPLT | SPO11 |
| | | | | | | | [313,332] |
| 8 | 1089 | LTSPSMEIKEVASIILHKDF | PPSSSS [129,148] | 23 | 2482 | LATSHMDFSTKSVFSKFAQL | TAG-1 |
| | | | | | | | [208,227] |
| 8 | 1090 | KRQIYVAAFTVQAAAETLSE | PSMA [729,748] | 23 | 2483 | YPRIISDVLIDEHLVLKSAS | TDRD1 |
| | | | | | | | [880,899] |
| 8 | 1091 | NILMLHEVVFDRKSGSLALI | RAGE-1 [62,81] | 23 | 2484 | WNSQEFIQTLSDDFISVRER | TEX14 |
| | | | | | | | [1245,1264] |
| 8 | 1092 | SQPTPDNVLSAVTPELINLA | SAGE1 [402,421] | 23 | 2485 | LSRKLDKAVVHLKKAHRRVH | TEX15 |
| | | | | | | | [993,1012] |
| 8 | 1093 | QVISVTFEKALNAGFIQATD | SART3 [397,416] | 23 | 2486 | SVLDNITTDKILIDVFDGLP | TPTE [452,471] |
| 8 | 1094 | KDKRDYLWTSAKNTLSTPLP | SCP-1 [833,852] | 23 | 2487 | AVQELRRQNYSSNAYHMSST | TSGA10 |
| | | | | | | | [644,663] |
| 8 | 1095 | QEEKILNMFRQQQKILQQSR | SCP3a [146,165] | 23 | 2488 | SPLTGIADASQSSMHNALHI | TYR [349,368] |
| 8 | 1096 | DRDEAWVLETIGKYWAAEKV | SCPN1 [161,180] | 24 | 2489 | RHLDLSNNSLVSLTYVSFRN | 5T4 [237,256] |
| 8 | 1097 | KKIRTTLQKIRTQMFKBEIR | se57-1 [38,57] | 24 | 2490 | IEPLESSVGFEHVIYQVKHK | ADAM2 |
| | | | | | | | [127,146] |
| 8 | 1098 | DISSGLVAIFIAFYGDRKKV | SLCO6A1 [152,171] | 24 | 2491 | FQTKAATVTKELRESSLLNQ | AFP [202,221] |
| 8 | 1099 | QEMRQFFTVGQQPQIPITTG | SOX-6 [719,738] | 24 | 2492 | DEKSGDASRLTSAFPDSLYE | AKAP-3 |
| | | | | | | | [697,716] |
| 8 | 1100 | REILREQPDNIPAFAAAYFE | SP17 [25,44] | 24 | 2493 | SEMMVSLMKTLKVHSSGKPI | AKAP-4 |
| | | | | | | | [342,361] |
| 8 | 1101 | YLSKAERFKMMLTLISKGQK | SPAG1 [875,894] | 24 | 2494 | NYDEKRQLSLNINKLPGDKL | BRDT [511,530] |
| 8 | 1102 | AQRKRFTRVEMARVLMERNQ | SPAG9 [498,517] | 24 | 2495 | DQLGNEYFRQPPPRSPPLIH | CAGE1 |
| | | | | | | | [176,195] |
| 8 | 1103 | EVLEERTRIQFIRWSHTRIF | SPATA19 [106,125] | 24 | 2496 | QTALQKTQLQLQEMAQKATH | CCDC62 |
| | | | | | | | [104,123] |
| 8 | 1104 | SSDYLSRVYLPNKLKFGGWI | SPO11 [377,396] | 24 | 2497 | KSLKSQVAEAKMTFKIFQMN | CTAGE5 |
| | | | | | | | [181,200] |
| 8 | 1105 | SYVYMKRNYKAMTKLGFKVT | SSX-1 [47,66] | 24 | 2498 | KKGRSKKAHVLAASVEQATQ | CTNNA2 [50,69] |
| 8 | 1106 | WEKMKASEKIFYVYMKRKYE | SSX-2 [37,56] | 24 | 2499 | SRERGSGIRSVSFYEHIITV | DCAF12 |
| | | | | | | | [336,355] |
| 8 | 1107 | FGRLQGIFPKIMPKKPAEEG | SSX-3 [101,120] | 24 | 2500 | LVLAGVGFFIHRRRKNQRAR | EPHA2 |
| | | | | | | | [549,568] |
| 8 | 1108 | EVMTKLGFKVTLPPFMRSKR | SSX-4 [56,75] | 24 | 2501 | KLARQATNLKVNFFFERIMK | FBXO39 |
| | | | | | | | [275,294] |
| 8 | 1109 | RGKHAWTHRVRERKQLVIYE | SSX-5 [159,178] | 24 | 2502 | LSAASPTISSFSPRLENRNN | FSIP1 [330,349] |
| 8 | 1110 | KERISALNLQIEEEKNKQRQ | SYCE1 [134,153] | 24 | 2503 | KFKKAMTIGDVSLVQELLDS | GASZ [49,68] |
| 8 | 1111 | KQKNEKLISLQEQLQRFLKK | TAF7L [443,462] | 24 | 2504 | HQVRSFFQRLQPGLKWVPET | Glypican-3 |
| | | | | | | | [36,55] |
| 8 | 1112 | QALAPDFRLNPVRRLIPAAR | TAG-1 [413,432] | 24 | 2505 | PSGSWSQKRSFVYVWKTWGQ | gp100 |
| | | | | | | | [139,158] |
| 8 | 1113 | SEDDQWYRASVLAYASEESV | TDRD1 [553,572] | 24 | 2506 | STDKVIVFVSRKAVADHLSS | HAGE [486,505] |
| 8 | 1114 | IMLLERSIMAKEGPLKVAQT | TEKT5 [374,393] | 24 | 2507 | DMRRLIDLGVGLAPYSAVEK | JARID1B |
| | | | | | | | [937,956] |
| 8 | 1115 | KDRTMNLQDIRYILKNDLKD | TEX14 [497,516] | 24 | 2508 | AIIGKQGQHIKQLSRFAGAS | KOC1 [419,438] |
| 8 | 1116 | ALKSRISWEGLLALDNGEME | TEX15 [712,731] | 24 | 2509 | ILETKELNDLHIEALAVIAN | KU-CT-1 |
| | | | | | | | [240,259] |
| 8 | 1117 | KPKPHVSDHNRLLHLARPKA | THEG [289,308] | 24 | 2510 | AEPLFEQNNSLNVNFNTQKK | LIPI [77,96] |
| 8 | 1118 | DSKLYIPLEYRSISLAIALF | TPTE [117,136] | 24 | 2511 | DLVKLKYLEYRQVPNSNPAR | MAGE-B3 |
| | | | | | | | [251,270] |
| 8 | 1119 | DDERMEQMSNMTLMKETIST | TSGA10 [155,174] | 24 | 2512 | NMLGIYDGKRHLIFGEPRKL | MAGE-B4 |
| | | | | | | | [225,244] |
| 8 | 1120 | RGQRPRTSAPSRAGALLLLL | TSPSO [10,29] | 24 | 2513 | PGSPSFSSTLLSLFQSSPER | MAGE-C1 |
| | | | | | | | [376,395] |
| 8 | 1121 | ESEQAALGEEAVLLLDDIMA | TSPY1 [53,72] | 24 | 2514 | RLSTLKFIGQYGNGLKSGSM | MORC1 |
| | | | | | | | [89,108] |
| 8 | 1122 | KSYLEQASRIWSWLLGAAMV | TYR [465,484] | 24 | 2515 | KSSGQMAQKFSFSKVFGPAT | MPHOSPH1 |
| | | | | | | | [104,123] |
| 8 | 1123 | LLLRTPYSSDNLYQMTSQLE | WT1 [215,234] | 24 | 2516 | KQKFSRLWSSKSVTEIHLYF | NLRP4 |
| | | | | | | | [104,123] |
| 8 | 1124 | KIESQRIISGRISGYISEAS | ZNF16S [201,220] | 24 | 2517 | NEWNYTRAGQAFTMLQTEGK | NXF2 [597,616] |
| 9 | 1125 | AYPEKMRNRVLLELNSADLD | 5T4 [324,343] | 24 | 2518 | KEILEAEIESHHPRLASAVQ | NY-BR-1 |
| | | | | | | | [1132,1151] |
| 9 | 1126 | DENSYWRNQNPGSLLQLPHT | ACRBP [303,322] | 24 | 2519 | LMAEDEPSGALLKPLVFRVD | NYD-TSPG |
| | | | | | | | [73,92] |
| 9 | 1127 | HPDTFSYPIERGRILNWEGV | ACTL8 [59,78] | 24 | 2520 | DEMNKEIEAARRQFQSQLAD | ODF2 [576,595] |
| 9 | 1128 | DVAFLLVYREKSNYVGATFQ | ADAM2 [264,283] | 24 | 2521 | DSVELGAAGASAQPLQPSSP | PASD1 |
| | | | | | | | [345,364] |
| 9 | 1129 | LFGLEIWTNKNLIVVDDVRK | ADAM29 [246,264] | 24 | 2522 | RRGWLYKQESTGMKLWKKRW | PEPP2 |
| | | | | | | | [172,191] |
| 9 | 1130 | QLAVSVILRVAKGYQELLEK | AFP [364,383] | 24 | 2523 | WYDFFIVSQAVRSGSVSPTH | PIWIL1 |
| | | | | | | | [774,793] |
| 9 | 1131 | LMTDTQFVSAVKRTVFSHGS | AKAP-3 [348,367] | 24 | 2524 | LLPELSFMTGIPEKMKKDFR | PIWIL2 |
| | | | | | | | [493,512] |
| 9 | 1132 | KEFADSISKGLMVYANQVAS | AKAP-4 [323,342] | 24 | 2525 | ALLDHEAKKMSTYLKTISPN | PIWIL3 |
| | | | | | | | [733,752] |
| 9 | 1133 | EVEMLKIRSEFKRKYGKSLY | ANXA2 [297,316] | 24 | 2526 | VAESSSNTSSRLSVIVVRKK | PIWIL4 |
| | | | | | | | [715,734] |
| 9 | 1134 | DEIEIDFETLKASTLRELEK | BRDT [550,569] | 24 | 2527 | LAKFSPYLGQMINLRRLLLS | PRAME |
| | | | | | | | [249,268] |
| 9 | 1135 | EYSEKTTQFPSVYAVPGTEQ | CABYR [114,133] | 24 | 2528 | NDQLMFLERAFIDPLGLPER | PSMA [665,684] |
| 9 | 1136 | KNKSVSQYLEMEKTLSKKEE | CAGE1 [431,450] | 24 | 2529 | MPAMSTREQHATIIHNLREE | SAGE1 |
| | | | | | | | [378,397] |
| 9 | 1137 | NIGAIGLELWQVRSGTIFEN | CALR3 [296,315] | 24 | 2530 | EEDEVKAARTRRKVLSRAVA | SART3 [25,44] |
| 9 | 1138 | LHNLRQIYVKQQSDLQFLNF | CCDC62 [282,301] | 24 | 2531 | RVQAENSRLEMHFKLKEDYE | SCP-1 [229,248] |
| 9 | 1139 | KNDLYPNPKPEVLHMIYMRA | CDCA1 [33,52] | 24 | 2532 | QQPHGVEGKLSSINNMGLNS | SOX-6 [517,536] |
| 9 | 1140 | MMQKPGSNAPVGGNVTSSFS | CT45 [84,103] | 24 | 2533 | GTTKTFKIPIEHLDFKYIEK | SPAG1 [13,32] |
| 9 | 1141 | ISTEHQSLVLVKRLLAVSVS | CT46 [20,39] | 24 | 2534 | EQKREQYRQVKAHVQKEDGR | SPAG9 |
| | | | | | | | [623,642] |
| 9 | 1142 | PPWEQDYRMMETPPGQSYPD | CTAGE2 [566,585] | 24 | 2535 | ARNEAPAFTIDNRSSWENIK | SPO11 [63,82] |
| 9 | 1143 | SVRSRLYVGREKKLALMLSG | CTAGE5 [65,84] | 24 | 2536 | EPVQQEMNGILLGYEIRYWK | TAG-1 |
| | | | | | | | [834,853] |
| 9 | 1144 | DSKLAVSLAETTGLIKLEEE | CTCFL [166,185] | 24 | 2537 | LQKQVEKHEHILLFLLNNST | TDRD1 |
| | | | | | | | [1147,1166] |
| 9 | 1145 | NAVVLTVKASYVASTKYQKV | CTNNA2 [874,893] | 24 | 2538 | LEAQLHEYVKQGNYVKVKKI | TEX14 [23,42] |
| 9 | 1146 | FIWRAISITPVHKSSSGFQD | CXorf48 [229,248] | 24 | 2539 | LVELQMMMETIQFIENKKRH | TEX15 |
| | | | | | | | [1690,1709] |
| 9 | 1147 | LEHTLLSKGFRGASPHRKST | CXorf61 [94,113] | 24 | 2540 | AARVSPISESVLARLSKFEV | TPTE [49,68] |
| 9 | 1148 | PQRPRNRPYFQRRRQQAPGP | DBPC [316,335] | 24 | 2541 | EIQGNVKVLKSERDKIFLLY | TSGA10 [49,68] |
| 9 | 1149 | VVEVQTSQITKIPILKDREP | DCAF12 [114,133] | 25 | 2542 | FLLVYREKSNYVGATFQGKM | ADAM2 |
| | | | | | | | [267,286] |
| 9 | 1150 | LEGGHWLSEKRHRLQAIRDG | DKKL1 [184,203] | 25 | 2543 | EKNIFLASFVHEYSRRHPQL | AFP [346,365] |
| 9 | 1151 | PPSYLGQSVPQFFTFEDAPS | DMRT1 [296,315] | 25 | 2544 | FRGQERPDEFTASVSEGIMT | AKAP-3 |
| | | | | | | | [268,287] |
| 9 | 1152 | SRKRKAVTKRARLQRSYEMN | DPPA2 [147,166] | 25 | 2545 | SQKMDMSNIVLMLIQKLLNE | AKAP-4 |
| | | | | | | | [621,640] |
| 9 | 1153 | DSKSLRTALQKEITTRYQLD | EpCAM [158,177] | 25 | 2546 | RRRREAMVGTIDMTLQSDIM | BRDT [920,939] |
| 9 | 1154 | RARRPKFADIVSILDKLIRA | EPHA2 [858,877] | 25 | 2547 | LEKRVKELQMKITKQQVFID | CAGE1 |
| | | | | | | | [333,352] |
| 9 | 1155 | SRFFIDFPHIEEQQKKIESY | FAM46D [264,283] | 25 | 2548 | LHDELLFTVEREKRKDELLN | CCDC62 |
| | | | | | | | [250,269] |
| 9 | 1156 | LVIAEMMELGSRSRGASQKK | FATE1 [32,51] | 25 | 2549 | SENGAYLDNPPKGALKKLIH | CTAGE5 |
| | | | | | | | [299,318] |
| 9 | 1157 | EDYFSHHLAVYNSPQFKKTM | FBXO39 [201,220] | 25 | 2550 | LANRFKEFGKEMVKLNYVAA | CTNNA2 |
| | | | | | | | [172,191] |
| 9 | 1158 | EMENTKKFLSLTAVSEETVG | FSIP1 [168,187] | 25 | 2551 | NKNKELGAVSLEGYFHLWKA | DCAF12 |
| | | | | | | | [262,281] |
| 9 | 1159 | DKMEHAQKLMRLQNLRGGHI | FTHL17 [62,81] | 25 | 2552 | EKDGEFSVLQLVGMLRGIAA | EPHA2 |
| | | | | | | | [706,725] |
| 9 | 1160 | GHLITAVQNVITELPVNSQK | GASZ [368,387] | 25 | 2553 | ALRVFKARIYTNRYETNEED | FBXO39 |
| | | | | | | | [395,414] |
| 9 | 1161 | QGNHVPPYYGNSVRATVQRY | GATA-3 [56,75] | 25 | 2554 | KELDSQLQDATQKMKKLEKT | FSIP1 [109,128] |
| 9 | 1162 | KMEEKYQLTARLNMEQLLQS | Glypican-3 [76,95] | 25 | 2555 | QLLQSASMELKFLITQNAAV | Glypican-3 |
| | | | | | | | [91,110] |
| 9 | 1163 | MEVTVYHRRGSRSYVPLAHS | gp100 [184,203] | 25 | 2556 | LADTNSLAVVSTQLIMPGQE | gp100 |
| | | | | | | | [571,590] |
| 9 | 1164 | LMPGFIHLVLQPSLKGQRNR | HAGE [297,316] | 25 | 2557 | PSLKGQRNRPGMLVLITTRE | HAGE [308,327] |
| 9 | 1165 | PYNDYFEYFGPDFKLHISPS | HDAC1 [329,348]; | 25 | 2558 | LTESKETASAMATLGEARLR | JARID1B |
| | | | HDAC2 [330,349] | | | | [1132,1151] |
| 9 | 1166 | SPTNMQGFTKSLNAFNVGDD | HDAC3 [74,93] | 25 | 2559 | RIRKLQTRNIPPHLQWEVLD | KOC1 [79,98] |
| 9 | 1167 | EVSESPGSIQVARGQPAVLP | IGPS11 [24,43] | 25 | 2560 | DLRAVLLINSKSYVSPPSSM | KU-CT-1 |
| | | | | | | | [597,616] |
| 9 | 1168 | TVEYELKYRNIGSETWKTII | IL13RA2 [66,85] | 25 | 2561 | KNLLKHGASLENFHFIGVSL | LIPI [162,181] |
| 9 | 1169 | FKSDYFNMPVHMVPTELVEK | JARID1B [389,408] | 25 | 2562 | ALSTTTSVDVSYKKSYKGAN | MAGE-B3 |
| | | | | | | | [68,87] |
| 9 | 1170 | QFEQSETETVHLFIPALSVG | KOC1 [399,418] | 25 | 2563 | VAARRGTTAMTSAYSRATSS | MAGE-B4 |
| | | | | | | | [321,340] |
| 9 | 1171 | AAYNKLLNNNLSLKYSQTGY | KU-CT-1 [542,561] | 25 | 2564 | DVQSPLQNPASSFFSSALLS | MAGE-C1 |
| | | | | | | | [123,142] |
| 9 | 1172 | ITMPFSSPMEAELVRRILSR | Lage-1 [92,111] | 25 | 2565 | EVKKAPEAVKIAESILKEAQ | MORC1 |
| | | | | | | | [288,307] |
| 9 | 1173 | WKNIHKQVIQSAYETIKLKG | LDHC [227,246] | 25 | 2566 | YLAYDETLNVLKFSATAQKV | MPHOSPH1 |
| | | | | | | | [460,479] |
| 9 | 1174 | AARAPSTRITYGTITKERDY | LEMD1 [118,137] | 25 | 2567 | GLRKTDFDEETQALLTAKEE | NLRP4 |
| | | | | | | | [956,975] |
| 9 | 1175 | NFNTQKKTVWLIHGYRPVGS | LIPI [90,109] | 25 | 2568 | SLSALPKTQHDLSSILVDVW | NXF2 [470,489] |
| 9 | 1176 | SDPARYEFLWGPRALAETSY | MAGE-A1 [257,276] | 25 | 2569 | VPNKAFELKNEQTLRADPMF | NY-BR-1 |
| | | | | | | | [477,496] |
| 9 | 1177 | GSDPRSFPLWYEEALKDEEE | MAGE-A10 | 25 | 2570 | DETTPAVVQSVLLERGWNKF | NYD-TSPG |
| | | | [320,339] | | | | [92,111] |
| 9 | 1178 | LLTQNWVQEKYLVYRQVFGT | MAGE-A11 | 25 | 2571 | TVTKSHKRGMKGETVNVRRS | GDP2 [39,58] |
| | | | [358,377] | | | | |
| 9 | 1179 | AREPFTKAEMLGSVIRNFQD | MAGE-A12 | 25 | 2572 | GASAQPLQPSSPVAYDITSQ | PASD1 |
| | | | [126,145] | | | | [353,372] |
| 9 | 1180 | APEEKIWEELSMLEVFEGRE | MAGE-A2 [216,235] | 25 | 2573 | YTLEQALLSASQETEMHADN | PEPP2 |
| | | | | | | | [664,683] |
| 9 | 1181 | KASSSLQLVEGIELMEVEPI | MAGE-A3 [153,172] | 25 | 2574 | KSQGLQISAGFQELSLAERG | PIWIL1 [61,80] |
| 9 | 1182 | PRALAETSYVKVLEHVVRVN | MAGE-A4 [276,295]; | 25 | 2575 | NFYDPTSAMVLQQHRLQIWP | PIWIL2 |
| | | | MAGE-A8 [278,297] | | | | [342,361] |
| 9 | 1183 | RKVAKLVHFLLLKYRAREPV | MAGE-A6 [111,130] | 25 | 2576 | NKLIGSTVLTKYNNKTYRVD | PIWIL3 |
| | | | | | | | [316,335] |
| 9 | 1184 | VAELVRFLLRKYQIKEPVTK | MAGE-A8 [116,135] | 25 | 2577 | ILPSNQKTYYDSIKKYLSSD | PIWIL4 |
| | | | | | | | [550,569] |
| 9 | 1185 | NYKRYFPVIFGKASEFMQVI | MAGE-A9 [141,160] | 25 | 2578 | DELFSYLIEKVKRKKNVLRL | PRAME |
| | | | | | | | [189,208] |
| 9 | 1186 | DHFTEILNGASRRLELVFGL | MAGE-B1 [144,163] | 25 | 2579 | YDVLLSYPNKTHPNYISTIN | PSMA [113,132] |
| 9 | 1187 | TKGEMLKIVGKRFREHFPEI | MAGE-B2 [133,152] | 25 | 2580 | AFINMAATGVSSMSTREQYA | SAGE1 |
| | | | | | | | [604,623] |
| 9 | 1188 | ILKKASFNMEVVFGVELKKV | MAGE-B3 [152,171] | 25 | 2581 | YVEFKEEKSALQALEMERKS | SART3 |
| | | | | | | | [750,769] |
| 9 | 1189 | EPTTKAEMLKITSKKYKEHF | MAGE-B4 [128,147] | 25 | 2582 | SEEETLKTLYRNNNPPASHL | SCP-1 [905,924] |
| 9 | 1190 | EYKPYETQILNRTSQHLVVA | MAGE-B6 [228,247] | 25 | 2583 | KQIEQLYAAQLASMQVSPGA | SOX-6 [364,383] |
| 9 | 1191 | PVSSSFSYTLLSLPQSSPER | MAGE-C1 [446,465] | 25 | 2584 | RKDKPAATAASFTAKEWEKT | SPAG1 [76,95] |
| 9 | 1192 | EEPVTEAEMLMIVIKYKDYF | MAGE-C2 [159,178] | 25 | 2585 | ESLFEELSSAGSGLIGDVDE | SPAG9 |
| | | | | | | | [380,399] |
| 9 | 1193 | RAQLRLDFIHANSTTHSFLF | MORC1 [10,29] | 25 | 2586 | IPLTKREQMKLESILRRPYV | SPO11 |
| | | | | | | | [329,348] |
| 9 | 1194 | EENIGILPRTLNVLETSLQE | MPHOSPH1 | 25 | 2587 | SPPATYRWKMNGTEMKLEPG | TAG-1 [66,85] |
| | | | [166,185] | | | | |
| 9 | 1195 | ITYYQELQRDISEMFLQTYK | MUC-1 [1063,1082] | 25 | 2588 | RPAKSKKLNKLVENSLSISN | TDRD1 |
| | | | | | | | [135,154] |
| 9 | 1196 | HQKYLSAPERAHLAKNLKLT | NKX3.1 [145,164] | 25 | 2589 | QLYNWAAPEVILQKAATVKS | TEX14 |
| | | | | | | | [418,437] |
| 9 | 1197 | SFFSDFGLMWYLEELKKEEF | NLRP4 [4,23] | 25 | 2590 | ELQTYHDQLVELLEETKREK | TEX15 |
| | | | | | | | [1758,1777] |
| 9 | 1198 | SDEGMEVIERLIYLYHKEHQ | NR6A1 [362,381] | 25 | 2591 | FHNRVVRIMIDDHNVPTLHQ | TPTE [295,314] |
| 9 | 1199 | PSLSQEQQEMVQAFSAQSGM | NXF2 [566,585] | 25 | 2592 | KQKVQDTNLEVNKLKNILKS | TSGA10 |
| | | | | | | | [386,405] |
| 9 | 1200 | QLKVLIAENTMLTSKLKEKQ | NY-BR-1 | 26 | 2593 | RVSGSSQSPPNLKYKSTLKI | AKAP-3 |
| | | | [1111,1130] | | | | [201,220] |
| 9 | 1201 | STQSQALGSLRTTTPAFTLN | NYD-TSPG [9,28] | 26 | 2594 | DEAVGKVARKQLLDWLLANL | AKAP-4 |
| | | | | | | | [835,854] |
| 9 | 1202 | KNYEGMIDNYKSQVMKTRLE | ODF2 [537,556] | 26 | 2595 | LQLPDYYTIIKNPMDLNTIK | BRDT [61,80] |
| 9 | 1203 | TKYVETSAPSHSISARTKAF | ODF3 [112,131] | 26 | 2596 | NVEELIEDKYKIILEKNETK | CAGE1 |
| | | | | | | | [360,379] |
| 9 | 1204 | STLMLFPINIWIFELERNVS | ODF4 [157,176] | 26 | 2597 | DAPAFNEKASIVLPSQDDFS | CCDC62 |
| | | | | | | | [610,629] |
| 9 | 1205 | DQASETTSMEWDTQVVKGSS | OIP5 [29,48] | 26 | 2598 | RANRDYVEKQVQEAIAGISN | CTNNA2 |
| | | | | | | | [237,256] |
| 9 | 1206 | TSNSEAISSSSIPQFPITSD | PASD1 [658,677] | 26 | 2599 | WVYRGEAERIFIELKFTVRD | EPHA2 [85,104] |
| 9 | 1207 | NDHYRSVYQKRLMDEAKILK | PBK [71,90] | 26 | 2600 | QRREKEIKKQGLEMRIKLWE | FSIP1 [133,152] |
| 9 | 1208 | RSVPAGLTLQSVSPQSLQGK | PEPP2 [595,614] | 26 | 2601 | ELKFLIIQNAAVFQEAFEIV | Glypican-3 |
| | | | | | | | [99,118] |
| 9 | 1209 | LQFYNIIFRRLLKIMNLQQI | PIWIL1 [209,228] | 26 | 2602 | EKVPVSEVMGTTLAEMSTPE | gp100 |
| | | | | | | | [377,396] |
| 9 | 1210 | RTEGGLELLADVSHKVIRND | PIWIL2 [369,388] | 26 | 2603 | GRTGRAGRTGVSITTLTRND | HAGE [576,595] |
| 9 | 1211 | KKMSTYLKTISPNNFTLAFI | PIWIL3 [740,759] | 26 | 2604 | NLNNMPVMEQSVLAHITADI | JARID1B |
| | | | | | | | [460,479] |
| 9 | 1212 | VAGSMGENVEYPKIIKVQEN | PIWIL4 [509,528] | 26 | 2605 | VNTESETAVVNVTYSSKDQA | KOC1 [113,132] |
| 9 | 1213 | SISISALQSLLQHLIGLSNL | PRAME [417,436] | 26 | 2606 | KIPKEKLPDFSWELHISELK | KU-CT-1 |
| | | | | | | | [758,777] |
| 9 | 1214 | LYSEELFPEELSVVLGTNEL | PRSS55 [110,129] | 26 | 2607 | ERMEGRPLRTTVELDTSGTY | LIPI [334,353] |
| 9 | 1215 | RPFYRHVIYAPSSHNKYAGE | PSMA [684,703] | 26 | 2608 | RAGREHFAFGEPRELLTKVW | MAGE-C1 |
| | | | | | | | [1029,1048] |
| 9 | 1216 | VYSKQPYTEYISTRWYRAPE | RAGE-1 [152,171] | 26 | 2609 | PLNSFQYQRRQAMGIPFIIQ | MORC1 |
| | | | | | | | [466,485] |
| 9 | 1217 | NVLSGLINMAGASIPAMSSR | SAGE1 [459,478] | 26 | 2610 | VSLESNSNSKILNVKRATIS | MPHOSPH1 |
| | | | | | | | [502,521] |
| 9 | 1218 | RTRRKVLSRAVAAATYKTMG | SART3 [33,52] | 26 | 2611 | FVKQAVNLLQEANFHIIDNV | NLRP4 |
| | | | | | | | [565,584] |
| 9 | 1219 | GAIRKMREDRWAVIAKMDRK | SCP-1 [946,965] | 26 | 2612 | VEMRDVHKDQQLRHTPYSIR | NXF2 [65,84] |
| 9 | 1220 | LQKKIMMETQQQEIASVRKS | SCP3a [211,230] | 26 | 2613 | ELEVKQQLEQALRIQDIELK | NY-BR-1 |
| | | | | | | | [1006,1025] |
| 9 | 1221 | EIEALLGMELVRLGLERGET | SCRN1 [103,122] | 26 | 2614 | STDVLVKRKLVHDIIDLIYL | NYD-TSPG |
| | | | | | | | [398,417] |
| 9 | 1222 | EKEKRTLLERKLSLENKLLQ | se57-1 [199,218] | 26 | 2615 | QLADLQQLPDILKITEAKLA | ODF2 [592,611] |
| 9 | 1223 | PGSTRIKARKRKQLHFETSR | SLCO6A1 [340,359] | 26 | 2616 | VAENISSLLGHLPAEIVGKK | PASD1 [57,76] |
| 9 | 1224 | EGSDQHVASHLPLHPIMHNK | SOX-6 [31,50] | 26 | 2617 | TLRKTKKMMDLRTERPRSAV | PEPP2 |
| | | | | | | | [868,887] |
| 9 | 1225 | GKYSAAIALLEPAGSEIADD | SPAG1 [466,485] | 26 | 2618 | NEYMPSRIIVYREGVGDGQL | PIWIL1 |
| | | | | | | | [690,709] |
| 9 | 1226 | KTREGGSVVGASVEYKDVAG | SPAG9 [699,718] | 26 | 2619 | RTLGRGSSDASLLPLGRAAG | PIWIL2 |
| | | | | | | | [142,161] |
| 9 | 1227 | QDIHVTREVVKHHLSKSELL | SPATA19 [81,100] | 26 | 2620 | ERRIGGVFQDLVVNTRQDMK | PIWIL3 [92,111] |
| 9 | 1228 | ADSKMKAEIQALTELSSDYL | SPO11 [362,381] | 26 | 2621 | QLYQYHVTYIPDLASRRLRI | PIWIL4 |
| | | | | | | | [122,141] |
| 9 | 1229 | AFDDIATYFSKKEWKKMKYS | SSX-1 [24,43] | 26 | 2622 | SRYISMSVWTSPRRLVELAG | PRAME [12,31] |
| 9 | 1230 | PNRGNQVERPQMTFGRLQGI | SSX-2 [88,107] | 26 | 2623 | LQDFDKSNPIVLRMMNDQLM | PSMA [650,669] |
| 9 | 1231 | AQIPEKIQKAFDDIAKYFSK | SSX-3 [15,34] | 26 | 2624 | GIPSMSTKELYATVTQNVHE | SAGE1 |
| | | | | | | | [518,537] |
| 9 | 1232 | WEKMKSSEKIVYVYMKLNYE | SSX-4 [37,56] | 26 | 2625 | NEQRMKAAEKEAALVQQEEE | SART3 |
| | | | | | | | [577,596] |
| 9 | 1233 | NGDDAFVRRPRVGSQIPEKM | SSX-5 [2,21] | 26 | 2626 | KMKELTELLEESRDKVNQLE | SCP-1 [281,300] |
| 9 | 1234 | KIEDLMEMVQKLQKVGSLEP | SYCE1 [32,51] | 26 | 2627 | SLYSERNTSTSPHKPDEGSR | SOX-6 [88,107] |
| 9 | 1235 | KDKLKIDLLPDGRHAVVEVE | TAF7L [126,145] | 26 | 2628 | RSGQFAEAAGKYSAAIALLE | SPAG1 |
| | | | | | | | [457,476] |
| 9 | 1236 | TEGRHEVSQTTGNLYIARTN | TAG-1 [179,198] | 26 | 2629 | LMPLVVAVLENLDSVFAQDQ | SPAG9 [50,69] |
| 9 | 1237 | TADELRMISSTFLNLPFQGI | TDRD1 [807,826] | 26 | 2630 | SSSEVLASIENTIQDIITSL | SPO11 [43,62] |
| 9 | 1238 | LQVRGAEASRLWASRLTDDS | TEKT5 [100,119] | 26 | 2631 | FAFGNPVPRIKWRKVEGSLS | TAG-1 |
| | | | | | | | [262,281] |
| 9 | 1239 | LVYERITIGTLFSVLHERRS | TEX14 [324,343] | 26 | 2632 | NSGKLLDHVLIEMGYGLKPS | TDRD1 |
| | | | | | | | [431,450] |
| 9 | 1240 | KQRFRGMLWFDLSLLPELVQ | TEX15 [2085,2104] | 26 | 2633 | KAVVSGNYLEADVRLPKPYY | TEX14 |
| | | | | | | | [466,485] |
| 9 | 1241 | PSTTMTKARKRRRRRRLMEL | THEG [112,131] | 26 | 2634 | NIHTSLAIAQKLMELKLGKI | TEX15 |
| | | | | | | | [351,370] |
| 9 | 1242 | MDVLLRVFVERRQQYFSDLF | TPTE [139,158] | 26 | 2635 | TYSIPRYVRELKIQIEMEKK | TPTE [421,440] |
| 9 | 1243 | DKKSENIASLGESLAMKEKT | TSGA10 [278,297] | 26 | 2636 | RMEQMSNMTLMKETISTVEK | TSGA10 |
| | | | | | | | [158,177] |
| 9 | 1244 | IDQMTQTASDVPVLQVIMHS | TSP50 [171,190] | 27 | 2637 | KETTIATILLKKVLLKHAKE | AKAP-3 |
| | | | | | | | [309,328] |
| 9 | 1245 | TPESAPEELLAVQVELEPVN | TSPY1 [104,123] | 27 | 2638 | MMAYSETTMMSDDIDWLRSH | AKAP-4 [1,20] |
| 9 | 1246 | FLLHHAFVDSIFEQWLRRHR | TYR [386,405] | 27 | 2639 | GLHNYYDVVKNPMDLGTIKE | BRDT [305,324] |
| 9 | 1247 | ELVRHHNMHQRNMTKLQLAL | WT1 [430,449] | 27 | 2640 | KKTLQNLEEVLANTQKHLQE | CAGE1 |
| | | | | | | | [379,398] |
| 9 | 1248 | GRAFSQSSNLSQHQRIHMRE | ZNF165 [462,481] | 27 | 2641 | ITLSSIFTKELVEKHNLPWS | CCDC62 |
| | | | | | | | [393,412] |
| 10 | 1249 | AFARRPPLAELAALNLSGSR | 5T4 [110,129] | 27 | 2642 | STKYQKVYGTAAVNSPVVSW | CTNNA2 |
| | | | | | | | [887,906] |
| 10 | 1250 | LQNETYSALSPGKSEDVVLR | ACRBP [510,529] | 27 | 2643 | VMWEVMTYGERPYWELSNHE | EPHA2 |
| | | | | | | | [806,825] |
| 10 | 1251 | SNTYQLPDGSRVELTPMQRV | ACTL8 [235,254] | 27 | 2644 | EGDQSGWVVPVKGYELAVTQ | FSIP1 [297,316] |
| 10 | 1252 | VILAQLLSLSMGITYDDINK | ADAM2 [309,328] | 27 | 2645 | VIYTQLMNPGLPDSALDINE | Glypican-3 |
| | | | | | | | [177,196] |
| 10 | 1253 | PFGGQKHIIHIKVKKLLFSK | ADAM29 [57,76] | 27 | 2646 | PDGQVIWVNNTIINGSQVWG | gp100 [98,117] |
| 10 | 1254 | LQTMKQEFLINLVKQKPQIT | AFP [545,564] | 27 | 2647 | PEELVSMAERFKAHQQKREM | HAGE [614,633] |
| 10 | 1255 | YDSDSWAEDLIVSALLLIQY | AKAP-3 [518,537] | 27 | 2648 | HVERKILDLFQLNKLVAEEG | JARID1B |
| | | | | | | | [122,141] |
| 10 | 1256 | DIMEAMLKRLVSALIGEEKE | AKAP-4 [421,440] | 27 | 2649 | PKDVFDPLMIESKKAATVVL | KU-CT-1 [13,32] |
| 10 | 1257 | AQRQDIAFAYQRRTKKELAS | ANXA2 [66,85] | 27 | 2650 | ATTFIYNRAVKNTRKVAVSL | LIPI [138,157] |
| 10 | 1258 | PNHHQLAFNYQELEHLQTVK | BRDT [732,751] | 27 | 2651 | SVLQIPVSAASSSTLVSIFQ | MAGE-C1 |
| | | | | | | | [161,180] |
| 10 | 1259 | PAQLAAQMLGKVSSIHSDQS | CABYR [172,191] | 27 | 2652 | GVNVENRSQAGMFIYSNNRL | MORC1 |
| | | | | | | | [357,376] |
| 10 | 1260 | SDEAKSIRDVPTLLGAKLEK | CAGE1 [674,693] | 27 | 2653 | ILQIEDSEMSRVIRVSELSL | MPHOSPH1 |
| | | | | | | | [359,378] |
| 10 | 1261 | QDWEKHFLDASTSKQSDWNG | CALR3 [226,245] | 27 | 2654 | LKKEEFRKFKEHLKQMTLQL | NLRP4 [18,37] |
| 10 | 1262 | TSKLQRLLAESRQMVTDLEL | CCDC62 [631,650] | 27 | 2655 | LSDITEKAPKVKTLNLSKNK | NXF2 [288,307] |
| 10 | 1263 | MIVKKEKLATAQFKINKKHE | CDCA1 [345,364] | 27 | 2656 | AFKPAIEMQNSVPNKAFELK | NY-BR-1 |
| | | | | | | | [466,485] |
| 10 | 1264 | AQLQRTPMSALVFPNKISTE | CT46 [4,23] | 27 | 2657 | YNEDDSVVEKAVSVRFEAAP | NYD-TSPG |
| | | | | | | | [465,484] |
| 10 | 1265 | VAEAKMTFKRFQANEERLEI | CTAGE2 [157,176] | 27 | 2658 | HELAETEHENTVLRHNIERM | ODF2 [167,186] |
| 10 | 1266 | MAATEISVLSEQFTKIKELE | CTCFL [1,20] | 27 | 2659 | DKVNPKSSQRKLNWIPSFPT | PASD1 [10,29] |
| 10 | 1267 | ADMADVMRLLSHLKIVEEAL | CTNNA2 [141,160] | 27 | 2660 | EEETRGVISYQTLPRNMPSH | PEPP2 |
| | | | | | | | [427,446] |
| 10 | 1268 | VEEDKPHYGLRAIKVDVVPR | CXorf48 [80,99] | 27 | 2661 | IDVEVTRPEWYDFFIVSQAV | PIWIL1 |
| | | | | | | | [765,784] |
| 10 | 1269 | SGLINSNTENNLAVYDLSRD | CXorf61 [46,65] | 27 | 2662 | AFEGSILYLPVKLQQVLELK | PIWIL2 |
| | | | | | | | [273,292] |
| 10 | 1270 | VPVKGSRYAPNRRKSRRFIP | DBPC [166,185] | 27 | 2663 | GSKITYIDYYRQQHKEIVTV | PIWIL3 |
| | | | | | | | [352,371] |
| 10 | 1271 | FHLWKAENTLSKLLSTKLPY | DCAF12 [276,295] | 27 | 2664 | LKKKRITENSEAQLAHLIPEL | PIWIL4 |
| | | | | | | | [360,379] |
| 10 | 1272 | KMTENKTGEVLISENVVASI | DKKL1 [108,127] | 27 | 2665 | PMQDIKMILKMVQLDSIEDL | PRAME |
| | | | | | | | [219,238] |
| 10 | 1273 | TYYSSFYQPSLFPYYNNLYN | DMRT1 [208,227] | 27 | 2666 | FSTQKVKMHIHSTNEVTRIY | PSMA [337,356] |
| 10 | 1274 | VRTYWIIIELKHKAREKPYD | EpCAM [139,158] | 27 | 2667 | PPEELHAAAYVFTNDGQQMR | SAGE1 [13,32] |
| 10 | 1275 | PPQQSRVWKYEVTYRKKGDS | EPHA2 [460,479] | 27 | 2668 | RQKMSEIFPLTEELWLEWLH | SART3 |
| | | | | | | | [135,154] |
| 10 | 1276 | EQQKKIESYLHNHFIGEGMT | FAM46D [275,294] | 27 | 2669 | ESKKKRKMAFEFDINSESSE | SCP-1 [877,896] |
| 10 | 1277 | PNTKAEMEMSLAEELNHGRQ | FATE1 [6,25] | 27 | 2670 | SKEWKEKMERLNTSELLGEI | SOX-6 [158,177] |
| 10 | 1278 | FERIMKYERLARILLQEIPI | FBXO39 [289,308] | 27 | 2671 | HPFSMKPLLRRAMAYETLEQ | SPAG1 |
| | | | | | | | [517,536] |
| 10 | 1279 | PVKGYELAVTQHQQLAEIDI | FSIP1 [306,325] | 27 | 2672 | NEQLITQYFREKALRKHAFE | SPAG9 [82,101] |
| 10 | 1280 | EAGLAEYLFEKLTLGGRVKE | FTHL17 [163,182] | 27 | 2673 | AVPSNIQGIRNLVTDAKFVL | SPO11 |
| | | | | | | | [202,221] |
| 10 | 1281 | FEDHIFSSYTAFGELEVFLH | GASZ [265,284] | 27 | 2674 | STGILSVRDATKITLAFSSA | TAG-1 |
| | | | | | | | [499,518] |
| 10 | 1282 | SSFNPAALSRHMSSLSHISP | GATA-3 [390,409] | 27 | 2675 | KEILPNGHVKVHFVEYGNIE | TDRD1 |
| | | | | | | | [785,804] |
| 10 | 1283 | ARRELKVFGNFPKLIMTQVS | Glypican-3 | 27 | 2676 | PPPSQELLDDIELLKQQQGS | TEX14 |
| | | | [201,220] | | | | [1331,1350] |
| 10 | 1284 | SLIYRRRLMKQDFSVPQLPH | gp100 [613,632] | 27 | 2677 | ENQIDTAFLSSTSKYEKLEK | TEX15 |
| | | | | | | | [1188,1207] |
| 10 | 1285 | IKNIQSTTNTTIQIIQEQPE | HAGE [90,109] | 27 | 2678 | KQKARRIYPSDFAVEILFGE | TPTE [520,539] |
| 10 | 1286 | TYETAVALDTEIPNELPYND | HDAC1 [313,332] | 27 | 2679 | QSQIALQEKESEIQLLKEHL | TSGA10 |
| | | | | | | | [580,599] |
| 10 | 1287 | LDQIRQTIFENLKMLNHAPS | HDAC3 [355,374] | 28 | 2680 | VFSHGSQKATDIMDAMLRKL | AKAP-3 |
| | | | | | | | [362,381] |
| 10 | 1288 | LGQSFSFHSGNANIPSIYAN | IGFS11 [341,360] | 28 | 2681 | NSSEDSEDERVKRLAKLQEQ | BRDT [410,429] |
| 10 | 1289 | VNGSSENKPIRSSYFTFQLQ | IL13RA2 [214,233] | 28 | 2682 | NIENYSTNALIQFVDTISIS | CAGE1 [97,116] |
| 10 | 1290 | SIPVHLNSLPRLETLVAEVQ | JARID1B | 28 | 2683 | ELNDMVAVHQQQLLSWEEDR | CCDC62 [43,62] |
| | | | [1048,1067] | | | | |
| 10 | 1291 | FEVKLEAHIRVPSFAAGRVI | KOC1 [484,503] | 28 | 2684 | NEFENKIILDPMTFSEARFR | CTNNA2 |
| | | | | | | | [281,300] |
| 10 | 1292 | LQEPSDLRAVLLINSKSYVS | KU-CT-1 [592,611] | 28 | 2685 | IVSILDKLIRAPDSLKTLAD | EPHA2 |
| | | | | | | | [867,886] |
| 10 | 1293 | EALFKKSAETLWNIQKDLIF | LDHC [313,332] | 28 | 2686 | GSRSSNASLEVLSTEPGSFK | ESIP1 [23,42] |
| 10 | 1294 | LQEVKILAQFYNDFVNISSI | LIPI [402,421] | 28 | 2687 | FTDVSLYILGSDINVDDMVN | Glypican-3 |
| | | | | | | | [149,168] |
| 10 | 1295 | AREEVIKAEMLESVIKNYKH | MAGE-A1 [119,138] | 28 | 2688 | RSYVPLAHSSSAFTITDQVP | gp100 |
| | | | | | | | [195,214] |
| 10 | 1296 | LVQFLLFKYQMKEPITKAEI | MAGE-A10 | 28 | 2689 | LLMEAQLLQVSLPEIQELYQ | JARID1B |
| | | | [141,160] | | | | [1349,1368] |
| 10 | 1297 | TVRPADLTRVIMPLEQRSQH | MAGE-A11 | 28 | 2690 | PVIQLLALKTLGVIANDKES | KU-CT-1 |
| | | | [100,119] | | | | [205,224] |
| 10 | 1298 | PRKLLTQDLVQENYLEYRQV | MAGE-A12 | 28 | 2691 | FSFKLLNQLGMIEEPRLYEK | LIPI [376,395] |
| | | | [242,261] | | | | |
| 10 | 1299 | FEGPRMFPDLESEFQAAISR | MAGE-A2 [92,111] | 28 | 2692 | QSPPEGENTHSPLQIVPSLP | MAGE-C1 |
| | | | | | | | [518,537] |
| 10 | 1300 | GSVVGNWQYFFPVIFSKASS | MAGE-A3 [137,156] | 28 | 2693 | EGELEQTETYLEALLKEDNL | MORC1 |
| | | | | | | | [937,956] |
| 10 | 1301 | GSNPARYEFLWGPFALAFTS | MAGE-A4 [264,283] | 28 | 2694 | NMANSIKFSVWVSFFEIYNE | MPHOSPH1 |
| | | | | | | | [262,281] |
| 10 | 1302 | KASDSLQLVFGIELMEVDPI | MAGE-A6 [153,172] | 28 | 2695 | HYEEQQAWNITLRIFQKMDR | NLRP4 [62,81] |
| 10 | 1303 | AEMLESVIKNYKRYFPVIFG | MAGE-A9 [132,151] | 28 | 2696 | LLRRTKRDIVDSLSALFKTQ | NXF2 [459,478] |
| 10 | 1304 | LNGASRRLELVFGLELKEEN | MAGE-B1 [150,169] | 28 | 2697 | ANILIDSGADINLVDVYGNT | NY-BR-1 [66,85] |
| 10 | 1305 | NSATEEEIWEFLNMLGVYDG | MAGE-B2 [216,235] | 28 | 2698 | TDINTLTRQKELLLQKLSTF | ODF2 [244,263] |
| 10 | 1306 | EPRKLITQBLVKLKYLEYRQ | MAGE-B3 [243,262] | 28 | 2699 | TQLLQQLYTSKAVSDEAVLT | PASD1 |
| | | | | | | | [178,197] |
| 10 | 1307 | LNMLGIYDGKRHLIFGEFRK | MAGE-B4 [224,243] | 28 | 2700 | KQESTGMKLWKKRWFVLSEL | PEPP2 |
| | | | | | | | [178,197] |
| 10 | 1308 | ARKIITEDLVQDKYVVYRQV | MAGE-B6 [328,347] | 28 | 2701 | LFLPKRLQQKVTEVFSKTRN | PIWIL1 |
| | | | | | | | [168,187] |
| 10 | 1309 | KQPITKAEMLTNVISRYTGY | MAGE-C1 [926,945] | 28 | 2702 | HRPSERQDNHGMLLKGEILL | PIWIL2 |
| | | | | | | | [474,493] |
| 10 | 1310 | PHSSPPYYEFLWGPRAHSES | MAGE-C2 [290,309] | 28 | 2703 | PMGITMKPAEMIEVEGDANS | PIWIL3 |
| | | | | | | | [535,554] |
| 10 | 1311 | LKSGSMRIGKEFILFTKKEE | MORC1 [103,122] | 28 | 2704 | ASRRLRIALLYSHSELSNKA | PIWIL4 |
| | | | | | | | [135,154] |
| 10 | 1312 | QKWREERDQLVAALEIQLKA | MPHOSPH1 | 28 | 2705 | RLSEGDVMHLSQSPSVSQLS | PRAME |
| | | | [1516,1535] | | | | [333,352] |
| 10 | 1313 | DNRPALGSTAPPVHNVTSAS | MUC-1 [943,962] | 28 | 2706 | SGKIVIARYGKVFRGNKVKN | PSMA [197,216] |
| 10 | 1314 | AFSHTQVIELERKFSHQKYL | NKX3.1 [130,149] | 28 | 2707 | RHSSSKRRKSMSSWLDKQED | SAGE1 [55,74] |
| 10 | 1315 | FKDPKRAMEAFNLVRESEQL | NLRP4 [315,334] | 28 | 2708 | YVEIWQAYLDYLRRRVDFKQ | SART3 |
| | | | | | | | [417,436] |
| 10 | 1316 | PSPGSTLSSSRSVELNGFMA | NP6A1 [190,209] | 28 | 2709 | AVPSQTVSRNFTSVPHGISK | SCP-1 [814,833] |
| 10 | 1317 | TMNKRYNVSQQALDLQNLRF | NXF2 [220,239] | 28 | 2710 | IGGSLGRGSSLDILSSLNSP | SOX-6 [468,487] |
| 10 | 1318 | IEVHNKASLTPLLLSITKRS | NY-BR-1 [109,128] | 28 | 2711 | NWREKLSPIPAVPASVPLQA | SPAG1 |
| | | | | | | | [575,594] |
| 10 | 1319 | SDFKDFIFEEMYIVQKYISN | NYD-TSPG [226,245] | 28 | 2712 | AATSPSTNGASPVMDKPPEM | SPAG9 |
| | | | | | | | [855,874] |
| 10 | 1320 | FKLENERLKASFAPMEDKLN | ODF2 [504,523] | 28 | 2713 | RKEMEIMADSKMKAEIQALT | SPO11 |
| | | | | | | | [355,374] |
| 10 | 1321 | RTGKELGPAYSILGRYQTKT | ODF3 [78,97] | 28 | 2714 | LVSSSAWSSALGSQTTFGPV | TAG-1 [19,38] |
| 10 | 1322 | YFNHKSFWSLILSHPSGAVS | ODF4 [201,220] | 28 | 2715 | YGNIEEVTADELRMISSTFL | TDRD1 |
| | | | | | | | [800,819] |
| 10 | 1323 | IVNASEMDIQNVPLSEKIAE | OIP5 [179,198] | 28 | 2716 | RVQRYGLHPDVNVYLGLTSE | TEX14 |
| | | | | | | | [530,549] |
| 10 | 1324 | SIPQFPITSDSTISTLETPQ | PASD1 [668,687] | 28 | 2717 | PSQSDLTLHSEIAYISKPGI | TEX15 |
| | | | | | | | [1550,1569] |
| 10 | 1325 | MDEAKILKSLHHPNIVGYRA | PBK [83,102] | 28 | 2718 | GVKTPSQKRYVAYFAQVKHL | TPTE [384,403] |
| 10 | 1326 | YNVTSDYAVHPMSPVGRTSR | PEPP2 [128,147] | 29 | 2719 | VSALLLIQYHLAQGGRREAR | AKAP-3 |
| | | | | | | | [529,548] |
| 10 | 1327 | RDWGLSFESNLLSFSGRILQ | PIWIL1 [439,458] | 29 | 2720 | KPGDDIVLMAQALEKLFMQK | BRDT [110,129] |
| 10 | 1328 | RINLKNTSFITSQELNWVKE | PIWIL2 [572,591] | 29 | 2721 | EERNKHLEDLIRKPREKARK | CAGE1 |
| | | | | | | | [730,749] |
| 10 | 1329 | DVSHKLLRIETAYDFIKRTS | PIWIL3 [283,302] | 29 | 2722 | NHPKVDIKREKNQKSLFKDQ | CCDC62 |
| | | | | | | | [337,356] |
| 10 | 1330 | PGFAISVSYFERKLLFSADV | PIWIL4 [244,263] | 29 | 2723 | DGVRDIRKAVLMIRTPEELE | CTNNA2 |
| | | | | | | | [618,637] |
| 10 | 1331 | KLPTLAKFSPYLGQMINLRR | PRAME [245,264] | 29 | 2724 | KEVPVAIKTLKAGYTEKQRV | EPHA2 |
| | | | | | | | [639,658] |
| 10 | 1332 | LVNYNLWIEKVTQLEGRPFN | PRSS55 [288,307] | 29 | 2725 | VVMVDREKKRLVELLKELDE | FSIP1 [269,288] |
| 10 | 1333 | LKKEGWRPRRTILFASWDAE | PSMA [405,424] | 29 | 2726 | YQLTARLNMEQLLQSASMEL | Glypican-3 |
| | | | | | | | [81,100] |
| 10 | 1334 | YVMELPKLKLSGVVRLSSYS | RAGE-1 [345,364] | 29 | 2727 | PSAAELQDLLDVSFEFEVEL | JARID1B |
| | | | | | | | [885,904] |
| 10 | 1335 | FGQNYERIFILLEEVQGSMK | SAGE1 [838,857] | 29 | 2728 | LVRGEYGRAWNEVMLQNDSR | KU-CT-1 |
| | | | | | | | [815,834] |
| 10 | 1336 | EKALNAGFIQATDYVEIWQA | SART3 [404,423] | 29 | 2729 | SLTDSESLIESEPLFTYTLD | MAGE-C1 |
| | | | | | | | [890,909] |
| 10 | 1337 | ESKQLNVYEIKVNKLELELE | SCP-1 [632,651] | 29 | 2730 | MKRSSSLPSWKSLLNVPMED | MORC1 |
| | | | | | | | [764,783] |
| 10 | 1338 | GEVQNMLEGVGVDINKALLA | SCP3a [68,87] | 29 | 2731 | LKLGIKHQSVAFTKLNNASS | MPHOSPH1 |
| | | | | | | | [330,349] |
| 10 | 1339 | FTGTPDPSRSIFKPFIFVED | SCRN1 [294,313] | 29 | 2732 | DLRRNGVVDADIPALLGTKI | NLRP4 |
| | | | | | | | [418,437] |
| 10 | 1340 | YESTSAHIIEETEYVKKIRT | se57-1 [23,42] | 29 | 2733 | PPEKPSAFEPAIEMQKSVPN | NY-BR-1 |
| | | | | | | | [698,717] |
| 10 | 1341 | RKRKQLHFFDSRLKELKLGT | SLCO6A1 [348,367] | 29 | 2734 | EQLHVQLADKELYVAEALST | ODF2 [421,440] |
| 10 | 1342 | TGGATVAEARVYRDARGRAS | SOX-6 [597,616] | 29 | 2735 | VEQYGPQENVHMFVESDSTY | PASD1 |
| | | | | | | | [247,266] |
| 10 | 1343 | IAKPNNAYEFGQIINALSTR | SPAG1 [800,819] | 29 | 2736 | DRPLTKINSVKLNSLPSEYE | PEPP2 |
| | | | | | | | [335,354] |
| 10 | 1344 | GYRNKIYVVQPKAMKIEKSF | SPAG9 [1063,1082] | 29 | 2737 | GVGDGQLKTLVNYEVPQFLD | PIWIL1 |
| | | | | | | | [703,722] |
| 10 | 1345 | PIMRDRIEQVRRSISRLTPV | SPATA19 [134,153] | 29 | 2738 | SINLTLTKWYSRVVFQMPHQ | PIWIL2 |
| | | | | | | | [763,782] |
| 10 | 1346 | DVLDRHRESLLAALRRGGRE | SPO11 [14,33] | 29 | 2739 | LAFIVVKKRINTRFFLKHGS | PIWIL3 |
| | | | | | | | [756,775] |
| 10 | 1347 | DNDHNRRIQVEHPQMTFGRL | SSX-1 [85,104] | 29 | 2740 | ASSSNGFLGTSRISTNEKYG | PIWIL4 [40,59] |
| 10 | 1348 | PNRGNQVQRPQMTFGRLQGI | SSX-3 [88,107] | 29 | 2741 | SLSGVMLTDVSPEPLQALLE | PRAME |
| | | | | | | | [355,374] |
| 10 | 1349 | HRLRERKQLVVYEEISDPEE | SSX-4 [166,185] | 29 | 2742 | MPEGELVYVNYARTEDFFKL | PSMA [169,188] |
| 10 | 1350 | QIPEKMQKAFDDIAKYFSEK | SSX-5 [16,35] | 29 | 2743 | TNEGQQMRSDEVNLVATGHQ | SAGE1 [25,44] |
| 10 | 1351 | LQIEEEKNKQRQLRLAFEEQ | SYCE1 [142,161] | 29 | 2744 | AHGTVKDLRLVTNRAGKPKG | SART3 |
| | | | | | | | [823,842] |
| 10 | 1352 | GSIPGFLISSGMSSHKQGHT | TAF7L [295,314] | 29 | 2745 | ESNKARAAHSFVVTEFETTV | SCP-1 [365,384] |
| 10 | 1353 | GLSYRWLLNEFPNFIPTEGR | TAG-1 [163,182] | 29 | 2746 | LNSPALFGRQDTVMKAIQEA | SOX-6 [484,503] |
| 10 | 1354 | IISEVLIDEHLVLKSASPHK | TDRD1 [883,902] | 29 | 2747 | LAITAPKELPMFLSNKLEGD | SPAG1 |
| | | | | | | | [829,848] |
| 10 | 1355 | FQAENTIMLLERSIMAKEGP | TEKT5 [368,387] | 29 | 2748 | MSERVSGLAGSIYREFERLI | SPAG9 [20,39] |
| 10 | 1356 | WKRLGWSESSRIIVLDQSRL | TEX14 [1476,1495] | 29 | 2749 | SGPSSKIRTREAAPSVAPKG | TAG-1 |
| | | | | | | | [697,716] |
| 10 | 1357 | TKREKNSYYVFLKYKRQVNE | TEX15 [1773,1792] | 29 | 2750 | EIIPLNRIYHLNRNIELFPP | TDRD1 |
| | | | | | | | [355,374] |
| 10 | 1358 | RSSLEYRASSRLKELAAPKI | THEG [221,240] | 29 | 2751 | DIPWKGLEGSVVKKAVVSGN | TEX14 |
| | | | | | | | [453,472] |
| 10 | 1359 | QKRYVAYFAQVKHLYNWNLP | TPTE [390,409] | 29 | 2752 | TPKKVEMQRSLPGSLLPLEN | TEX15 |
| | | | | | | | [2384,2403] |
| 10 | 1360 | ENELDSAHSEIELLRSQMAN | TSGA10 [540,559] | 29 | 2753 | HLFHQKRQLEKLIRRRVSEN | TPTE [207,226] |
| 10 | 1361 | RIGLLKLKQELKYSNYVRPI | TSP50 [206,225] | 30 | 2754 | SFYANRLTNLVIAMARKEIN | AKAP-3 |
| | | | | | | | [125,144] |
| 10 | 1362 | SNPYFQNKVITKEYLVNITE | TSPY1 [207,226] | 30 | 2755 | SVFPKTSISPLNVVQGASVN | BRDT [179,198] |
| 10 | 1363 | PAFLPWHRLFLLRWEQEIQK | TYR [205,224] | 30 | 2756 | LSKKEEEVERLQQLKKELEK | CAGE1 |
| | | | | | | | [445,464] |
| 10 | 1364 | AQYRIHTHGVFRGIQDVRRV | WT1 [284,303] | 30 | 2757 | QAFLRHPDVAATRANRDYVF | CTNNA2 |
| | | | | | | | [225,244] |
| 10 | 1365 | EHGQEIFQKKVSPPGPALNV | ZNF165 [142,161] | 30 | 2758 | LDDLAPDTTYLVQVQALTQE | EPHA2 |
| | | | | | | | [494,513] |
| 11 | 1366 | DERQNRSFEGMVVAALLAGR | 5T4 [188,207] | 30 | 2759 | LEMRIKLWEEIKSAKYSEAW | FSIP1 [144,163] |
| 11 | 1367 | TPTAKAWKYMEEEILGFGKS | ACRBP [340,359] | 30 | 2760 | NNYPSLTPQAFEFVGEFFTD | Glypican-3 |
| | | | | | | | [132,151] |
| 11 | 1368 | WEGSNRNFSVWLGASVVAHL | ACTL8 [328,347] | 30 | 2761 | PEDWENRTSYLHSPFSTGRS | JARID1B |
| | | | | | | | [1316,1335] |
| 11 | 1369 | VLIAIMVKVNFQRKKWRTED | ADAM2 [701,720] | 30 | 2762 | ISELKFQLKSNVIPIGHVKK | KU-CT-1 |
| | | | | | | | [773,792] |
| 11 | 1370 | ERNDSKLLEDLYVIVNIVDS | ADAM29 [215,234] | 30 | 2763 | ERTQSTFEGFAQSPLQIPVS | MAGE-C1 |
| | | | | | | | [219,238] |
| 11 | 1371 | QITEEQLEAVIADFSGLLEK | AFP [562,581] | 30 | 2764 | LPSIPLGTMSTISPSKNEKE | MORC1 |
| | | | | | | | [529,548] |
| 11 | 1372 | IDGHMSGQMVEHLMNSVMKL | AKAP-3 [661,680] | 30 | 2765 | NASSRSHSIFTVKILQIEDS | MPHOSPH1 |
| | | | | | | | [346,365] |
| 11 | 1373 | QVASEMMVSLMKTLKVHSSG | AKAP-4 [339,358] | 30 | 2766 | FFKDPKRAMEAFNLVRESEQ | NLRP4 |
| | | | | | | | [314,333] |
| 11 | 1374 | KELASALKSALSGHLETVIL | ANXA2 [81,100] | 30 | 2767 | VIEVHNKASLTPLLLSITKR | NY-BR-1 |
| | | | | | | | [108,127] |
| 11 | 1375 | ELEHLQTVKNISPLQILPPS | BRDT [743,762] | 30 | 2768 | APMEDKLNQAHLEVQQLKAS | ODF2 [516,535] |
| 11 | 1376 | AQLEENAKYSSVYMEAEATA | CABYR [366,385] | 30 | 2769 | LELMMEHLQKQPNTLRHVVI | PASD1 |
| | | | | | | | [402,421] |
| 11 | 1377 | SGEMLKFTEKSLAKSTAKES | CAGE1 [197,216] | 30 | 2770 | PAQTVHYRPINLSSSENKIV | PEPP2 |
| | | | | | | | [360,379] |
| 11 | 1378 | GTIFENFLITDDEEYADNFG | CALR3 [310,329] | 30 | 2771 | FRLTSRPQWALYQYHIDYNP | PIWIL1 |
| | | | | | | | [119,138] |
| 11 | 1379 | NFNVENSQELIQMYDSKMEE | CCDC62 [300,319] | 30 | 2772 | LVSMFRGLGIETVSKTPLKR | PIWIL2 [71,90] |
| 11 | 1380 | NYKEKMKETVQKLKNARQEV | CDCA1 [253,272] | 30 | 2773 | LLNAMPLHSWLILYSRSSHR | PIWIL3 |
| | | | | | | | [501,520] |
| 11 | 1381 | TERERMENIDSTILSPKQIK | CT46 [228,247] | 30 | 2774 | EKQLIGLIVLTRYNNRTYSI | PIWIL4 |
| | | | | | | | [294,313] |
| 11 | 1382 | HSEQNELMADISKRIQSLED | CTAGE2 [129,148] | 30 | 2775 | KYAGESFPGIYDALFDIESK | PSMA [699,718] |
| 11 | 1383 | EELERTIHSYQGQIISHEKK | CTAGE5 [440,459] | 30 | 2776 | INERTKYQLMKEVRRFGQNY | SAGE1 |
| | | | | | | | [823,842] |
| 11 | 1384 | EESEKYILTLQTVHFTSEAV | CTCFL [73,92] | 30 | 2777 | HVELIRLLRLEGELTKVRMA | SART3 |
| | | | | | | | [115,134] |
| 11 | 1385 | SLEERLESIISGAALMADSS | CTNNA2 [302,321] | 30 | 2778 | GSTLKFGAIRKMREDRWAVI | SCP-1 [940,959] |
| 11 | 1386 | SIYFSSEVVTGNVPLKVGQK | CXorf48 [56,75] | 30 | 2779 | GATVAEARVYRDARGRASSE | SOX-6 [599,618] |
| 11 | 1387 | ALIVFWKYRRFQRNTGEMSS | CXorf61 [14,33] | 30 | 2780 | GLQTANDSVNRLSRILMELD | SPAG1 |
| | | | | | | | [553,572] |
| 11 | 1388 | NPATAVSGTPAPPARSQADK | DBPC [68,87] | 30 | 2781 | ILENTQLLETKNALNIVKND | SPAG9 |
| | | | | | | | [414,433] |
| 11 | 1389 | ETWRNYFSDIEFFPNAVYTH | DCAF12 [405,424] | 30 | 2782 | EYQNGDGFGYLLSFRRQGST | TAG-1 |
| | | | | | | | [737,756] |
| 11 | 1390 | EVLISENVVASIQPAEGSFE | DKKL1 [116,135] | 30 | 2783 | SIKIVEILEEEVVTFAVEVE | TDRD1 |
| | | | | | | | [409,428] |
| 11 | 1391 | PQYSMALAADSASGEVGNPL | DMRT1 [229,248] | 30 | 2784 | SSTAQENTALETSSPIEEDF | TEX14 |
| | | | | | | | [1093,1112] |
| 11 | 1392 | HKAREKPYDSKSTRTALQKE | EpCAM [150,169] | 30 | 2785 | SYGENIVELSSSESSILLKE | TEX15 |
| | | | | | | | [1421,1440] |
| 11 | 1393 | MAAGYTAIEKVVQMTNEDIK | EPHA2 [926,945] | 30 | 2786 | VETGLPLYDDVKVQFFYSNL | TPTE [466,485] |

TABLE 25B
Hotspot Sequences and corresponding TAA

| | SEQ | | Source Antigen(s) | | SEQ | | Source Antigen(s) |
|---|---|---|---|---|---|---|---|
| | ID | | [AA position | | ID | | [AA position |
| Cycle | NO | Sequence | in Antigen] | Cycle | NO | Sequence | in Antigen] |
| 28 | 5432 | SVYADQVNIDYLMNRPQNLR | AKAP4 [157,176] | 11 | 5682 | SQVAGRLIIRAEELAQMWKV | Ropporin-1A |
| | | | | | | | [77-96] |
| 24 | 5433 | FTSSRMSSFNRHMKTHTSEK | CTCFL [264,283] | 11 | 5683 | YPEEIVRYYSPGYSETLLQR | SPAG6 [481- |
| | | | | | | | 500] |
| 31 | 5434 | KQQVFIDVINKLKENVEELI | CAGE1 [346,365] | 11 | 5684 | ESTEGSRSRSRSLDIQPSSE | SPAG8 [5-24] |
| 10 | 5435 | KKIEVYLRLHRHAYPEQRQD | DPPA2 [114,133] | 11 | 5685 | KSLPNENFQSLYNKELPVHI | TDRD4 [903- |
| | | | | | | | 922] |
| 15 | 5436 | GSKLGRRAKPEGALQNNEGL | EpCAM [75,94] | 11 | 5686 | FQQQQHQILATPLQNLQVLA | TTK [491-510] |
| 31 | 5437 | SETKEYFMSKTLGIGRLKRP | FSIP1 [516,535] | 11 | 5687 | ASAAARGFPLRTLNDWFSSM | WBP2NL [133- |
| | | | | | | | 152] |
| 16 | 5438 | KLHNINRPLTMKKEGIQTRN | GATA-3 [347,366] | 11 | 5688 | RTYLSQKSLQAHIKRRHKRA | ZNF645 [122- |
| | | | | | | | 141] |
| 20 | 5439 | SVMDLVGSILKNLRRVHPVS | LDHC [255,274] | 12 | 5689 | KWVGESERQLRLLFDQAYQM | ATAD2 [504- |
| | | | | | | | 523] |
| 22 | 5440 | QEESFSPTAMDAIFGSLSDE | MAGE-A11 | 12 | 5690 | KVFTHQGKVALYGKLVQSAQ | CASC5 [2009- |
| | | | [176,195] | | | | 2028] |
| 14 | 5441 | EDSVFAHPRKLLMQDLVQEN | MAGE-A2 [235,254] | 12 | 5691 | KQHREEDEVDSVLLSASKIL | CCDC110 [5- |
| | | | | | | | 24] |
| 18 | 5442 | REALDEKVAELVRFLLRKYQ | MAGE-A8 [109,128] | 12 | 5692 | ATYLWWVNNQSLPVSPRLQL | CEA [174-193] |
| 31 | 5443 | LQRPVSSFFSYTLASLLQSS | MAGE-C1 [775,794] | 12 | 5693 | LLKQQEEQTRVALLEQQMQA | CEP55 [339- |
| | | | | | | | 358] |
| 31 | 5444 | ESLVEKTPDEAASLVEGTSD | NY-BR-1 [262,281] | 12 | 5694 | SELDFPTQVIPLKTLNAVAS | EZH2 [87-106] |
| 28 | 5445 | DWNLYWRTSSFRMTEHNSVK | NYD-TSPG [120,139] | 12 | 5695 | SYGERHLDDLSLKILREEKK | HORMAD2 |
| | | | | | | | [57-76] |
| 3 | 5446 | SARVRSRSRGRGDGQEAPDV | PAGE4 [2,21] | 12 | 5696 | LRDAVVIEQSSSLNEASSGL | hTERT [786- |
| | | | | | | | 805] |
| 31 | 5447 | YLTGLITKMRNDFNVMKELA | PIWIL1 [388,407] | 12 | 5697 | PPMQLGFPSLHSFIKEHSLQ | KIF20A [511- |
| | | | | | | | 530] |
| 31 | 5448 | ENPAMQEKKRSSIWQFFSRL | SPAG9 [540,559] | 12 | 5698 | SLQARLFPGLAIKIQRSNGL | MCAK [6-25] |
| 32 | 5449 | NEQDLVREEAQKMSSLLPTM | SPAG9 [953,972] | 12 | 5699 | TRFFSRITKANVELLPRGAP | MSLN [138- |
| | | | | | | | 157] |
| 18 | 5450 | GFSYEQDPTLRDPEAVARRW | TSP50 [105,124] | 12 | 5700 | MWKVVNLPTELFNSVMNVGR | Ropporin-1A |
| | | | | | | | [93-112] |
| 1 | 5451 | RQLRLLETQAYQMRPSIIFF | ATAD2 [511-530] | 12 | 5701 | AVTNTLPVLLSLYMSTESSE | SPAG6 [375- |
| | | | | | | | 394] |
| 1 | 5452 | GVVFLALSAQLLQARLMKEE | BAGE-3 [4-23] | 12 | 5702 | YWPLRGKREAMLEMLLQHQI | SPAG8 [342- |
| | | | | | | | 361] |
| 1 | 5453 | RLLFSSSAAFAKFEITLFLS | CASC5 [2266- | 12 | 5703 | KFEDGIWYRAKVIGLPGHQE | TDRD4 [736- |
| | | | 2285] | | | | 755] |
| 1 | 5454 | QQLESFQALRMQTLQNVSMV | CCDC110 [61-80] | 12 | 5704 | EELSGRELTIDSIMNKVRDI | TTK [4-23] |
| 1 | 5455 | AQNTTYLWWVNGQSLPVSPR | CEA [527-546] | 12 | 5705 | NEGPPAGYRASPAGSGARPQ | WBP2NL [266- |
| | | | | | | | 285] |
| 1 | 5456 | KKRSEELLSQVQFLYTSLLK | CEP55 [322-341] | 12 | 5706 | RDHLSYIPPEQHTMVSLPSV | ZNF645 [174- |
| | | | | | | | 193] |
| 1 | 5457 | HPLLGVSATLNSVLNSNAIK | DKK1 [24-43] | 13 | 5707 | VLRMTRARRSQVEQQQLITV | ATAD2 [1293- |
| | | | | | | | 1312] |
| 1 | 5458 | TGEELFFDYRYSQADALKYV | EZH2 [718-737] | 13 | 5708 | TIREFFILLQVHILIQKPRQ | CASC5 [1895- |
| | | | | | | | 1914] |
| 1 | 5459 | MSWRGRSTYRPRPRRYVEPP | GAGE-2 [1-20] | 13 | 5709 | AKIQYLQNYLKESVQIQKKV | CCDC110 |
| | | | | | | | [426-445] |
| 1 | 5460 | EKRYLRMAVLTLYTDPMGSE | HORMAD2 [95- | 13 | 5710 | TPIISPPDSSYLSGANLNLS | CEA [595-614] |
| | | | 114] | | | | |
| 1 | 5461 | AERNRVATMPVRLLRDSPAA | HSPB9 [24-43] | 13 | 5711 | KLDRQHVQHQLHVILKELRK | CEP55 [367- |
| | | | | | | | 386] |
| 1 | 5462 | SFFYVTETTFQKNRLFFYRK | hTERT [559-578] | 13 | 5712 | KVMMVNGEHRIGIFAKRAIQ | EZH2 [698- |
| | | | | | | | 717] |
| 1 | 5463 | ANIRFSIWISFFEIYNELLY | KIF20A [295-314] | 13 | 5713 | GFHSMKVKVMTEATKVIDLE | HORMAD2 |
| | | | | | | | [226-245] |
| 1 | 5464 | KAQNASKGIYAMASRDVFLL | MCAK [365-384] | 13 | 5714 | DTDPRRLVQLLRQHSSPWQV | hTERT [442- |
| | | | | | | | 461] |
| 1 | 5465 | SQQNVSMDLATFMKLRTDAV | MSLN [520-539] | 13 | 5715 | VGQASFFNLTVKEMVKDVLK | KIF20A [131- |
| | | | | | | | 150] |
| 1 | 5466 | DEERELEKLFQLGPPSPVKM | PTTG-1 [150-169] | 13 | 5716 | FQIILRAKGRMHGKFSLVEL | MCAK [474- |
| | | | | | | | 493] |
| 1 | 5467 | STFQFLYTYIAKVDGEISAS | Ropporin-1A [159- | 13 | 5717 | DVLYPKARLAFQNMNGSEYF | MSLN [482- |
| | | | 178] | | | | 501] |
| 1 | 5468 | QNIETLQNAGVMSLLRTLLL | SPAG6 [32-51] | 13 | 5718 | EDFDPGVKEAAAWALRYIAR | SPAG6 [135- |
| | | | | | | | 154] |
| 1 | 5469 | FRHGHRGLLTMQLKSPMPSS | SPAG8 [309-328] | 13 | 5719 | ATAVNTRQRYYPMAGYIKED | TDRD4 [77-96] |
| 1 | 5470 | KMKSLEKISYVYMKRKYEAM | SSX-7 [39-58] | 13 | 5720 | VSDEKSSELIITDSITLKNK | TTK [340-359] |
| 1 | 5471 | GERVDVSKYLIKKGLALRER | TDRD4 [1087- | 13 | 5721 | PQRSEGSNVFSGRKTGTLFL | WBP2NL [34- |
| | | | 1106] | | | | 53] |
| 1 | 5472 | KKFAFVHISFAQFELSQGNV | TTK [140-159] | 13 | 5722 | IKRRHKRARKQVTSASLEKV | ZNF645 [134- |
| | | | | | | | 153] |
| 1 | 5473 | PKPEEKRFLLEEPMPFFYLK | URLC10 [95-114] | 14 | 5723 | ASLADVDPMQLDSSVRFDSV | ATAD2 [409- |
| | | | | | | | 428] |
| 1 | 5474 | RTLNDWFSSMGIYVITGEGN | WBP2NL [143-162] | 14 | 5724 | EITGMNTLLSAPIHTQMQQK | CASC5 [131- |
| | | | | | | | 150] |
| 1 | 5475 | IPPEQHTMVSLPSVQHMLQE | ZNF645 [180-199] | 14 | 5725 | KDKERQPFLVKQGSIISENE | CCDC110 |
| | | | | | | | [393-412] |
| 2 | 5476 | SRQDQIHSSIVSTLLALMDG | ATAD2 [542-561] | 14 | 5726 | ERVEGNRQIIGYVIGTQQAT | CEA [71-90] |
| 2 | 5477 | TALDVHFVSTLEPLSNAVKR | BAGE-3 [37-56] | 14 | 5727 | EKVAASPKSPTAALNESLVE | CEP55 [420- |
| | | | | | | | 439] |
| 2 | 5478 | WEQSLFSTTKPLFSSGQFSM | CASC5 [717-736] | 14 | 5728 | EWSGAEASMFRVLIGTYYDN | EZH2 [431- |
| | | | | | | | 450] |
| 2 | 5479 | LKFHSRVPRYTLSFLDQTKH | CCDC110 [371- | 14 | 5729 | VKVMTEATKVIDLENNLFRE | HORMAD2 |
| | | | 390] | | | | [232-251] |
| 2 | 5480 | AQYSWFVNGTFQQSTQELFI | CEA [267-286] | 14 | 5730 | EHRLREEILAKFLHWLMSVY | hTERT [533- |
| | | | | | | | 552] |
| 2 | 5481 | LTDKERHRLLEKIRVLEAEK | CEP55 [51-70] | 14 | 5731 | ERGIGQATHRFTFSQIFGPE | KIF20A [111- |
| | | | | | | | 130] |
| 2 | 5482 | STLEGYSRRTTLSSKMYHTK | DKK1 [163-182] | 14 | 5732 | EYTLNTLRYADRVKELSPHS | MCAK [573- |
| | | | | | | | 592] |
| 2 | 5483 | PRKFPSDKIFEAISSMFPDK | EZH2 [215-234] | 14 | 5733 | EDIRKWNVTSLETLKALLEV | MSLN [382- |
| | | | | | | | 401] |
| 2 | 5484 | KEPINVQVGFVSTGFHSMKV | HORMAD2 [213- | 14 | 5734 | AWALRYIARHNAELSQAVVD | SPAG6 [146- |
| | | | 232] | | | | 165] |
| 2 | 5485 | EELVVQVDGQWLMVTGQQQL | HSPB9 [66-85] | 14 | 5735 | LERSASTEKTLLNSSAVMLD | TDRD4 [125- |
| | | | | | | | 144] |
| 2 | 5486 | QQVWKNPTFFLRVISDTASL | hTERT [1023- | 14 | 5736 | ELRNLKSVQNSHFKEPLVSD | TTK [323-342] |
| | | | 1042] | | | | |
| 2 | 5487 | DSMEKVKVYLRVRPLLPSEL | KIF20A [60-79] | 14 | 5737 | ESTAAQAPENEASLPSASSS | WBP2NL [286- |
| | | | | | | | 305] |
| 2 | 5488 | ETASNEVVYRFTARPLVQTI | MCAK [317-336] | 14 | 5738 | PIQWETVSIFTRKHGNLTVD | ZNF645 [222- |
| | | | | | | | 241] |
| 2 | 5489 | DLPGRFVAESAEVLLPRLVS | MSLN [191-210] | 15 | 5739 | YQMRPSIIFFDEIDGLAPVR | ATAD2 [521- |
| | | | | | | | 540] |
| 2 | 5490 | ESNLLQSPSSILSTLDVELP | PUG-1 [175-194] | 15 | 5740 | KNDMDITKSYTIEINHRPLL | CASC5 [895- |
| | | | | | | | 914] |
| 2 | 5491 | IRVQPQDLIQWAADYFEALS | Ropporin-1A [26- | 15 | 5741 | RPLASELKGYFKVKDRTLKH | CCDC110 |
| | | | 45] | | | | [813-832] |
| 2 | 5492 | VKEAAAWALRYIARHNAELS | SPAG6 [141-160] | 15 | 5742 | GIQNELSVDHSDPVILNVLY | CEA [394-413] |
| 2 | 5493 | MQDGSESFFFRHGHRGLLTM | SPAG8 [300-319] | 15 | 5743 | KSETTLEKLKGEIAHLKTSV | CEP55 [22-41] |
| 2 | 5494 | RLQRIFPKIMPKKPAEEGND | SSX-7 [103-122] | 15 | 5744 | VEDETVLHNIPYMGDEVLDQ | EZH2 [122- |
| | | | | | | | 141] |
| 2 | 5495 | LNAAMNIARALQLSPSLRTY | TDRD4 [237-256] | 15 | 5745 | ITYLRGLFPESSYGERHLED | HORMAD2 |
| | | | | | | | [46-65] |
| 2 | 5496 | LYYMTYGKTPFQQIINQISK | TTK [724-743] | 15 | 5746 | REKRAERLTSRVKALFSVLN | hTERT [647- |
| | | | | | | | 666] |
| 2 | 5497 | LVVALPRVWTDANLTARQRD | URLC10 [8-27] | 15 | 5747 | KLNILKESLTSFYQEEIQER | KIF20A [625- |
| | | | | | | | 644] |
| 2 | 5498 | KLVFRNGDAIEFAQLMVKAA | WBP2NL [114-133] | 15 | 5748 | ESALAQQAKHFSALREVIKA | MCAK [684- |
| | | | | | | | 703] |
| 2 | 5499 | ELSLSLPFPIQWETVSIFTR | ZNF645 [214-233] | 15 | 5749 | EDLKALSQQNVSMDLATFMK | MSLN [514- |
| | | | | | | | 533] |
| 3 | 5500 | KKSQNYNIFQLENLYAVISQ | ATAD2 [1342- | 15 | 5750 | PPNILKHVVGQFSKVLPHDS | SPAG6 [425- |
| | | | 1361] | | | | 444] |
| 3 | 5501 | FVQNTLTKLLKERRKMQTVQ | BAGE-3 [82-101] | 15 | 5751 | KLAYIEPYKRTMQWSKEAKE | TDRD4 [792- |
| | | | | | | | 811] |
| 3 | 5502 | EHTTGQLTTMNRQIAVKVEK | CASC5 [814-833] | 15 | 5752 | SGTVNQIMMMANNPEDWLSL | TTK [49-68] |
| 3 | 5503 | SIEMEAMKTNILLIQDEKEM | CCDC110 [568- | 16 | 5753 | KEERTKFFEDLILKQAAKPP | ATAD2 [926- |
| | | | 587] | | | | 945] |
| 3 | 5504 | AYSGREIIYPNASLLIQNII | CEA [94-113] | 16 | 5754 | QAPPSSLLVHKLIFQYVEEK | CASC5 [2190- |
| | | | | | | | 2209] |
| 3 | 5505 | LREQLKARYSTTTLLEQLEE | CEP55 [87-106] | 16 | 5755 | NLKSKMKPLIFTTQSLIQKV | CCDC110 |
| | | | | | | | [454-473] |
| 3 | 5506 | LNSVLNSNAIKNLPPPLGGA | DKK1 [33-52] | 16 | 5756 | FVSNLATGRNNSIVKSITVS | CEA [656-675] |
| 3 | 5507 | KTLNAVASVPIMYSWSPLQQ | EZH2 [99-118] | 16 | 5757 | REQLKARYSTTTLLEQLEET | CEP55 [88- |
| | | | | | | | 107] |
| 3 | 5508 | LIRKLYILMQDLEPLPNNVV | HORMAD2 [160- | 16 | 5758 | SFLFNLNNEFVVDATRKGNK | EZH2 [664- |
| | | | 179] | | | | 683] |
| 3 | 5509 | RVSYRMSQKVHRKMLPSNLS | HSPB9 [92-111] | 16 | 5759 | VTEMYQFKFKYTKEGATMDF | HORMAD2 |
| | | | | | | | [116-135] |
| 3 | 5510 | RVKALFSVLNYERARRPGLL | hTERT [657-676] | 16 | 5760 | FQKNRLFFYRKSVWSKLQSI | hTERT [568- |
| | | | | | | | 587] |
| 3 | 5511 | STYDETLHVAKFSAIASQLV | KIF20A [489-508] | 16 | 5761 | KQIRQEEMKKLSLLNGGLQE | KIF20A [215- |
| | | | | | | | 234] |
| 3 | 5512 | ILRAKGRMHGKFSLVDLAGN | MCAK [477-496] | 16 | 5762 | RAKGRMHGKFSLVDLAGNER | MCAK [479- |
| | | | | | | | 498] |
| 3 | 5513 | LAFQNMNGSEYFVKIQSFLG | MSLN [490-509] | 16 | 5763 | TVAEVQKLLGPHVEGLKAEE | MSLN [543- |
| | | | | | | | 562] |
| 3 | 5514 | DAYPEIEKFFPFNPLEFESF | PTTG-1 [109-128] | 16 | 5764 | LNPDAKLKHQILSALSQVSK | SPAG6 [219- |
| | | | | | | | 238] |
| 3 | 5515 | GEISASHVSRMLNYMEQEVI | Ropporin-1A [173- | 16 | 5765 | SDILELGARIFVSSIKNGMW | TDRD4 [461- |
| | | | 192] | | | | 480] |
| 3 | 5516 | SLAEQNRFYKKAAAFVLRAV | SPAG6 [91-110] | 16 | 5766 | TYGKTPFQQIINQISKLHAI | TTK [728-747] |
| 3 | 5517 | IPPGFRNLVADRVPNYTSWS | SPAG8 [213-232] | 17 | 5767 | LHNHSAASATGSLELSSDFL | ATAD2 [10-29] |
| 3 | 5518 | KHAWTHRLRERKQLVIYEEI | SSX-7 [161-180] | 17 | 5768 | SFADTIKVFQTESHMKIVRK | CASC5 [60-79] |
| 3 | 5519 | NPWEKLSIHLYFDGMSLSYF | TDRD4 [1330- | 17 | 5769 | TLEFEMRHLQREYLSLSDKI | CCDC110 |
| | | | 1349] | | | | [759-778] |
| 3 | 5520 | NQTLESYRNEIAYLNKLQQH | TTK [562-581] | 17 | 5770 | QHTQELFISNITEKNSGLYT | CEA [457-476] |
| 3 | 5521 | KAASAAARGFPLRTLNDWFS | WBP2NL [131-150] | 17 | 5771 | IAELESKTNTLRLSQTVAPN | CEP55 [139- |
| | | | | | | | 158] |
| 3 | 5522 | QDVVTPNSVRSQVPALTTTY | ZNF645 [301-320] | 17 | 5772 | VYEFRVKESSIIAPAPAEDV | EZH2 [466- |
| | | | | | | | 485] |
| 4 | 5523 | VEKALAILSQPTPSLVVEHE | ATAD2 [1312- | 17 | 5773 | RTLEVQSDYSSYARTSIRAS | hTERT [938- |
| | | | 1331] | | | | 957] |
| 4 | 5524 | AQLLQARLMKEESPVVSWRL | BAGE-3 [12-31] | 17 | 5774 | QATHRFTFSQIFGPEVGQAS | KIF20A [116- |
| | | | | | | | 135] |
| 4 | 5525 | PVQNDLAYANDFASEYYLES | CASC5 [1360- | 17 | 5775 | QPDYDLETFVNKAESALAQQ | MCAK [671- |
| | | | 1379] | | | | 690] |
| 4 | 5526 | NSDILKNYNNFYRFLPTAPP | CCDC110 [190- | 17 | 5776 | GVLANPPNISSLSPRQLLGF | MSLN [50-69] |
| | | | 209] | | | | |
| 4 | 5527 | TQDATYLWWVNNQSLPVSPR | CEA [171-190] | 17 | 5777 | VAELATRPQNIETLQNAGVM | SPAG6 [24-43] |
| 4 | 5528 | LESLKQLHEFAITEPLVTFQ | CEP55 [394-413] | 17 | 5778 | QLIEGLDILFLLKTIEEFYK | TDRD4 [696- |
| | | | | | | | 715] |
| 4 | 5529 | SFGNDHSTLEGYSRRTTLSS | DKK1 [157-176] | 17 | 5779 | DPKQRISIPELLAHPYVQIQ | TTK [775-794] |
| 4 | 5530 | FRRADEVKSMFSSNRQKILE | EZH2 [32-51] | 18 | 5780 | RRSQVEQQQLITVEKALAIL | ATAD2 [1300- |
| | | | | | | | 1319] |
| 4 | 5531 | KEGVNSHFLLFDKEPINVQV | HORMAD2 [201- | 18 | 5781 | YSQDLGEMTKLNSKRVSFKL | CASC5 [1432- |
| | | | 220] | | | | 1451] |
| 4 | 5532 | EDNDHARDGFQMKLDAHGFA | HSPB9 [45-64] | 18 | 5782 | NENNRMSIEMEAMKTNILLI | CCDC110 |
| | | | | | | | [562-581] |
| 4 | 5533 | EAFTTSVRSYLPNTVTDALR | hTERT [113-132] | 18 | 5783 | NGNRTLTLFNVTRNDARAYV | CEA [551-570] |
| 4 | 5534 | FPSLHSFIKEHSLQVSPSLE | KIF20A [517-536] | 18 | 5784 | RHRLLEKIRVLEAEKEKNAY | CEP55 [56-75] |
| 4 | 5535 | MAMDSSLQARLFPGLAIKIQ | MCAK [1-20] | 18 | 5785 | NAVASVPIMYSWSPLQQNFM | EZH2 [102- |
| | | | | | | | 121] |
| 4 | 5536 | ATQMDRVNAIPFTYEQLDVL | MSLN [333-352] | 18 | 5786 | RAVRSLLRSHYREVLPLATF | hTERT [8-27] |
| 4 | 5537 | QIAHLPLSGVPLMILDEERE | PTTG-1 [135-154] | 18 | 5787 | KDVLKGQNWLIYTYGVTNSG | KIF20A [146- |
| | | | | | | | 165] |
| 4 | 5538 | IRAEELAQMWKVVNLPTELF | Ropporin-1A [85- | 18 | 5788 | QEHLVNSADDVIKMIDMGSA | MCAK [435- |
| | | | 104] | | | | 454] |
| 4 | 5539 | DILPQLVYSLAEQNRFYKKA | SPAG6 [83-102] | 18 | 5789 | QNVSMDLATFMKLRTDAVLP | MSLN [522- |
| | | | | | | | 541] |
| 4 | 5540 | DYRQEQPETFWIQRAPQLPV | SPAG8 [403-422] | 18 | 5790 | VVGQFSKVLPHDSKARRLFV | SPAG6 [432- |
| | | | | | | | 451] |
| 4 | 5541 | ISYVYMKRKYEAMTKLGFKA | SSX-7 [46-65] | 18 | 5791 | SVIKILEDNVLLVELFDSLG | TDRD4 [825- |
| | | | | | | | 844] |
| 4 | 5542 | WEEEAKVEFLKMVNNKAVSM | TDRD4 [590-609] | 18 | 5792 | SVVKDSQVGTVNYMPPEAIK | TTK [677-696] |
| 4 | 5543 | PYVQIQTHPVNQMAKGTTEE | TTK [789-808] | 19 | 5793 | QNIPSFAPVLLLATSDKPHS | ATAD2 [882- |
| | | | | | | | 901] |
| 4 | 5544 | NGDAIEFAQLMVKAASAAAR | WBP2NL [119-138] | 19 | 5794 | VQEIAEKQALAVGNKIVLHT | CASC5 [1234- |
| | | | | | | | 1253] |
| 4 | 5545 | EYNKEGKYYSKGVKLVRKKK | ZNF645 [12-31] | 19 | 5795 | LPTAPPNVMSQADTVILDKS | CCDC110 |
| | | | | | | | [204-223] |
| 5 | 5546 | GSSVKEVETYHRTRALRSLR | ATAD2 [59-78] | 19 | 5796 | SWRINGIPQQHTQVLFIAKI | CEA [626-645] |
| 5 | 5547 | EPEDGTALDVHFVSTLEPLS | BAGE-3 [32-51] | 19 | 5797 | LKDALEKNQQWLVYDQQREV | CEP55 [174- |
| | | | | | | | 193] |
| 5 | 5548 | EWSDDQAVFTFVYDTIQLTI | CASC5 [2139- | 19 | 5798 | FVVDATRKGNKIRFANHSVN | EZH2 [673- |
| | | | 2158] | | | | 692] |
| 5 | 5549 | NPQFSSEKNLVFGTRIEKEL | CCDC110 [97-116] | 19 | 5799 | IPQDRLTEVIASIIKPQNTY | hTERT [720- |
| | | | | | | | 739] |
| 5 | 5550 | DEPTISPSYTYYRPGVNLSL | CEA [416-435] | 19 | 5800 | QDQTLAELQNNMVLVKLDLR | KIF20A [785- |
| | | | | | | | 804] |
| 5 | 5551 | QQWLVYDQQREVYVKGLLAK | CEP55 [182-201] | 19 | 5801 | IKIQRSNGLIHSANVRTVNL | MCAK [17-36] |
| 5 | 5552 | GFINDEIFVELVNALGQYND | EZH2 [164-183] | 19 | 5802 | PIIRSIPQGIVAAWRQRSSR | MSLN [258- |
| | | | | | | | 277] |
| 5 | 5553 | EKVTEMYQFKFKYTKEGATM | HORMAD2 [114- | 19 | 5803 | MSQRQVLQVFEQYQKARTQF | SPAG6 [1-20] |
| | | | 133] | | | | |
| 5 | 5554 | ERNRVATMPVRLLRDSPAAQ | HSPB9 [25-44] | 19 | 5804 | SLPSPGELYAVQVKHVVSPN | TDRD4 [1406- |
| | | | | | | | 1425] |
| 5 | 5555 | TLTDLQPYMRQFVAHLQETS | hTERT [765-784] | 19 | 5805 | AKGTTEEMKYVLGQLVGLNS | TTK [802-821] |
| 5 | 5556 | IAEQYHTVLKLQGQVSAKKR | KIF20A [810-829] | 20 | 5806 | VRFDSVGGLSNHIAALKEMV | ATAD2 [423- |
| | | | | | | | 442] |
| 5 | 5557 | EVYVTFFEIYNGKLFDLLNK | MCAK [396-415] | 20 | 5807 | FPFLDKRYRKIVDVNFQSLL | CASC5 [2167- |
| | | | | | | | 2186] |
| 5 | 5558 | YPESVIQHLGYLFLKMSPED | MSLN [364-383] | 20 | 5808 | TAEENFLQEIKNAKSEASIY | CCDC110 |
| | | | | | | | [671-690] |
| 5 | 5559 | GLKLGSGPSIKALDGRSQVS | PUG-1 [23-42] | 20 | 5809 | VNEEATGQFRVYPELPKPSI | CEA [130-149] |
| 5 | 5560 | LHSQVAGRLIIRAEELAQMW | Ropporin-1A [75- | 20 | 5810 | IFELEKKTETAAHSLPQQTK | CEP55 [202- |
| | | | 94] | | | | 221] |
| 5 | 5561 | AHSENLAMAVIISKGVPQLS | SPAG6 [322-341] | 20 | 5811 | YMRLRQLKRFRRADEVKSMF | EZH2 [23-42] |
| 5 | 5562 | SSEESPRSALAAATAAAAAA | SPAG8 [35-54] | 20 | 5812 | YVVELLRSFFYVTETTFQKN | hTERT [552- |
| | | | | | | | 571] |
| 5 | 5563 | PEKIQKSFDDIAKYFSKKEW | SSX-7 [18-37] | 20 | 5813 | MSQGILSPPAGLLSEDDVVV | KIF20A [1-20] |
| 5 | 5564 | VVEKQFDQLLAFFDSRKKNL | TDRD4 [189-208] | 20 | 5814 | LLLVHEPKLKVELTKYLENQ | MCAK [288- |
| | | | | | | | 307] |
| 5 | 5565 | WERKSYWKNMLEAVHTIHQH | TTK [622-641] | 20 | 5815 | SRDPSWRQPERTILRPRFRR | MSLN [276- |
| | | | | | | | 295] |
| 5 | 5566 | SNVFSGRKTGTLFLTSYRVI | WBP2NL [40-59] | 20 | 5816 | SKHSVDLAEMVVEAEIFPVV | SPAG6 [237- |
| | | | | | | | 256] |
| 5 | 5567 | KRTYLSQKSLQAHIKRRHKR | ZNF645 [121-140] | 20 | 5817 | SILVQFVEYGSTAKLTLNRL | TDRD4 [1510- |
| | | | | | | | 1529] |
| 6 | 5568 | PASPARPRYRLSSAGPRSPY | ATAD2 [325-344] | 20 | 5818 | VERGAVPLEMLEIALRNLNL | TTK [170-189] |
| 6 | 5569 | LQARLMKEESPVVSWRLEPE | BAGE-3 [15-34] | 21 | 5819 | RKIRKKSNWYLGTIKKRRKI | ATAD2 [1164- |
| | | | | | | | 1183] |
| 6 | 5570 | NQDARILAMTPESIYSNPSI | CASC5 [429-448] | 21 | 5820 | AGKLNLSPSQYINEENLPVY | CASC5 [1726- |
| | | | | | | | 1745] |
| 6 | 5571 | LEALVKKLLPFRETVSKFHV | CCDC110 [315- | 21 | 5821 | ELKKHSQENIKFENSISRLT | CCDC110 |
| | | | 334] | | | | [721-740] |
| 6 | 5572 | SASGHSRTTVKTITVSAELP | CEA [482-501] | 21 | 5822 | QQSTQELFIPNITVNNSGSY | CEA [278-297] |
| 6 | 5573 | EEKEVLKQQLSAATSRIAEL | CEP55 [123-142] | 21 | 5823 | LKELRKARNQITQLESLKQL | CEP55 [381- |
| | | | | | | | 400] |
| 6 | 5574 | LQQNFMVEDETVLHNIPYMG | EZH2 [116-135] | 21 | 5824 | FLFNLNNDFVVDATRKGNKI | EZH2 [665- |
| | | | | | | | 684] |
| 6 | 5575 | QDLEPLPNNVVLTMKLHYYN | HORMAD2 [169- | 21 | 5825 | LWGSRHNERRFLRNTKKFIS | hTERT [477- |
| | | | 188] | | | | 496] |
| 6 | 5576 | VVQVDGQWLMVTGQQQLDVR | HSPB9 [69-88] | 21 | 5826 | LNGGLQEEELSTSLKRSVYI | KIF20A [228- |
| | | | | | | | 247] |
| 6 | 5577 | LGLEDIHRAWRTFVLRVRAQ | hTERT [681-700] | 21 | 5827 | SLRSRSTRMSTVSELRITAQ | MCAK [106- |
| | | | | | | | 125] |
| 6 | 5578 | ILIKQDQTLAELQNNMVLVK | KIF20A [781-800] | 21 | 5828 | SGKKAREIDESLIFYKKWEL | MSLN [304- |
| | | | | | | | 323] |
| 6 | 5579 | FVNKAESALAQQAKHFSALR | MCAK [679-698] | 21 | 5829 | ALALGRLANYNDDLAEAVVK | SPAG6 [62-81] |
| 6 | 5580 | LSTERVRELAVALAQKNVKL | MSLN [77-96] | 21 | 5830 | SLYLTGAVATIILQVDSEEN | TDRD4 [1051- |
| | | | | | | | 1070] |
| 6 | 5581 | NLLQSPSSILSTLEVELPPV | PTTG-1 [177-196] | 21 | 5831 | SEEEKKNLSASTVLTAQESF | TTK [196-215] |
| 6 | 5582 | NRAELTPELLKILHSQVAGR | Ropporin-1A [63- | 22 | 5832 | EIVVIGATNRLDSIDPALRR | ATAD2 [567- |
| | | | 82] | | | | 586] |
| 6 | 5583 | LQVFEQYQKARTQFVQMVAE | SPAG6 [7-26] | 22 | 5833 | ADGTSLDFSTYRSSQMESQF | CASC5 [1856- |
| | | | | | | | 1875] |
| 6 | 5584 | PQKQPPWEFLQVLEPGARGL | SPAG8 [239-258] | 22 | 5834 | STENLSSNIIIHPSENSDIL | CCDC110 |
| | | | | | | | [175-194] |
| 6 | 5585 | THRLRERKQLVIYEEISDPE | SSX-7 [165-184] | 22 | 5835 | PNASLLIQNIIQNDTGFYTL | CEA [103-122] |
| 6 | 5586 | VPKLSEFELIKMTNEIQSNL | TDRD4 [1199- | 22 | 5836 | GETENREKVAASPKSPTAAL | CEP55 [414- |
| | | | 1218] | | | | 433] |
| 6 | 5587 | GQNESFARIQVRFAELKAIQ | TTK [104-123] | 22 | 5837 | MFSSNRQKILERTEILNQEW | EZH2 [41-60] |
| 6 | 5588 | GWEGQATFKLVFRNGDAIEF | WBP2NL [106-125] | 22 | 5838 | RNTKKFISLGKHAKLSLQEL | hTERT [489- |
| | | | | | | | 508] |
| 6 | 5589 | SLSLPFPIQWETVSIFTRKH | ZNF645 [216-235] | 22 | 5839 | SPMFESTAADLGSVVRKNLL | KIF20A [21- |
| | | | | | | | 40] |
| 7 | 5590 | REDKVIPVTRSLRARNIVQS | ATAD2 [122-141] | 22 | 5840 | EEQVHSIRGSSSANPVNSVR | MCAK [170- |
| | | | | | | | 189] |
| 7 | 5591 | TPEDLMLSQYVYRPKIQIYR | CASC5 [1928- | 22 | 5841 | GLKAEERHRPVRDWILRQRQ | MSLN [557- |
| | | | 1947] | | | | 576] |
| 7 | 5592 | FHSRVPRYTLSFLDQTKHEM | CCDC110 [373- | 22 | 5842 | QEIKAEPGSLLQEYINSINS | SPAG6 [460- |
| | | | 392] | | | | 479] |
| 7 | 5593 | IIQNDTGFYTLHVIKSELVN | CEA [112-131] | 22 | 5843 | HFYIRKYSQIKDAKVLEKKV | TDRD4 [429- |
| | | | | | | | 448] |
| 7 | 5594 | VERQTITQLSFELSEFRRKY | CEP55 [250-269] | 22 | 5844 | LNKLQQHSDKIIRLYDYEIT | TTK [575-594] |
| 7 | 5595 | WRKRVKSEYMRLRQLKRFRR | EZH2 [15-34] | 23 | 5845 | KEMVVFPLLYPEVFEKFKIQ | ATAD2 [439- |
| | | | | | | | 458] |
| 7 | 5596 | DYQPLGFKEGVNSHFLLFEK | HORMAD2 [194- | 23 | 5846 | EENMDITKSHTVAIDNQIFK | CASC5 [507- |
| | | | 213] | | | | 526] |
| 7 | 5597 | QQQLDVREPERVSYRMSQKV | HSPB9 [82-101] | 23 | 5847 | NSISRLTEDKILLENYVRSI | CCDC110 |
| | | | | | | | [734-753] |
| 7 | 5598 | DYSSYARTSIRASLTFNRGF | hTERT [945-964] | 23 | 5848 | EPEIQNTTYLWWVNNQSLPV | CEA [346-365] |
| 7 | 5599 | SPYARILRSRRSPLLKSGPF | KIF20A [867-886] | 23 | 5849 | IPYMGDEVLDQDGTFIEELI | EZH2 [131- |
| | | | | | | | 150] |
| 7 | 5600 | PKESLRSRSTRMSTVSELRI | MCAK [103-122] | 23 | 5850 | HVRKAFKSHVSTLTELQPYM | hTERT [754- |
| | | | | | | | 773] |
| 7 | 5601 | DRVNAIPFTYEQLDVLKHKL | MSLN [337-356] | 23 | 5851 | DLKPLLSNEVIWLDSKQIRQ | KIF20A [200- |
| | | | | | | | 219] |
| 7 | 5602 | PALPKATRKALGTVNRATEK | PUG-1 [54-73] | 23 | 5852 | KAHTPFRESKLTQVLRDSFI | MCAK [534- |
| | | | | | | | 553] |
| 7 | 5603 | SVMNVGRFTEEIEWLKFLAL | Ropporin-1A [106- | 23 | 5853 | RELAVALAQKNVKLSTEQLR | MSLN [83- |
| | | | 125] | | | | 102] |
| 7 | 5604 | PTIQQTAALALGRLANYNDD | SPAG6 [55-74] | 23 | 5854 | VRYYSPGYSETLLQRVDSYQ | SPAG6 [486- |
| | | | | | | | 505] |
| 7 | 5605 | GSESFFFRHGHRGLLTMQLK | SPAG8 [303-322] | 23 | 5855 | KFKSQSLRSHFEKNTTLHYH | TDRD4 [650- |
| | | | | | | | 669] |
| 7 | 5606 | SGPKRGKHAWTHRLRERKQL | SSX-7 [155-174] | 23 | 5856 | NPEDWLSLLLKLEKNSVPLS | TTK [61-80] |
| 7 | 5607 | KIVAIKEFNPLSILVQFVEY | TDRD4 [1499- | 24 | 5857 | ISAKEFEVAMQKMIPASQRA | ATAD2 [674- |
| | | | 1518] | | | | 693] |
| 7 | 5608 | LSDALLNKLIGRYSQAIEAL | TTK [79-98] | 24 | 5858 | LLPNEIAIRPMEKTVLFTEN | CASC5 [1167- |
| | | | | | | | 1186] |
| 7 | 5609 | ALIPNGESLLKRSPNVELSF | WBP2NL [14-33] | 24 | 5859 | DEVDSVLLSASKILNSSEGV | CCDC110 [11- |
| | | | | | | | 30] |
| 7 | 5610 | DIQAPPPELSLSLPFPIQWE | ZNF645 [207-226] | 24 | 5860 | EPETQDATYLWWVNNQSLPV | CEA [168-187] |
| 8 | 5611 | KRYYLRQRKATVYYQAPLEK | ATAD2 [292-311] | 24 | 5861 | RQVYEFRVKESSIIAPAPAE | EZH2 [464- |
| | | | | | | | 483] |
| 8 | 5612 | PHVSKERIQQSLSNPLSISL | CASC5 [566-585] | 24 | 5862 | DGLRPIVNMEYVVGARTFRR | hTERT [628- |
| | | | | | | | 647] |
| 8 | 5613 | LKEFKKIISKYNVLQGQNKT | CCDC110 [505- | 24 | 5863 | DISMYGKEELLQVVEAMKTL | KIF20A [554- |
| | | | 524] | | | | 573] |
| 8 | 5614 | PRLQLSNENRTLTLLSVTRN | CEA [367-386] | 24 | 5864 | DEVAAINPELLQLLPLHPKD | MCAK [59-78] |
| 8 | 5615 | HQLHVILKELRKARNQITQL | CEP55 [375-394] | 24 | 5865 | LDQDQQEAARAALQGGGPPY | MSLN [215- |
| | | | | | | | 234] |
| 8 | 5616 | GPNAKSVQREQSLHSFHTLF | EZH2 [266-285] | 24 | 5866 | REIAKHTPELSQLVVNAGGV | SPAG6 [276- |
| | | | | | | | 295] |
| 8 | 5617 | STGFHSMKVKVMTEATKVID | HORMAD2 [224- | 24 | 5867 | NVWQPDAIEVLQQLLSKRQV | TDRD4 [1301- |
| | | | 243] | | | | 1320] |
| 8 | 5618 | YRMSQKVHRKMLPSNLSPTA | HSPB9 [95-114] | 24 | 5868 | NESFARIQVRFAELKAIQEP | TTK [106-125] |
| 8 | 5619 | RLPRLPQRYWQMRPLFLELL | hTERT [378-397] | 25 | 5869 | LSLEHIGRRRLRSAGAAQKK | ATAD2 [29-48] |
| 8 | 5620 | EGGILPRSLALIFNSLQGQL | KIF20A [176-195] | 25 | 5870 | NLNNLNGKTGEFLAFQTVHL | CASC5 [1586- |
| | | | | | | | 1605] |
| 8 | 5621 | ESSFPNWEFARMIKEFRATL | MCAK [224-243] | 25 | 5871 | EKNIQLSLEKQQMMEALDQL | CCDC110 |
| | | | | | | | [527-546] |
| 8 | 5622 | LLATQMERVNAIPFTYEQLD | MSLN [331-350] | 25 | 5872 | RNNSIVKSITVSASGTSPGL | CEA [664-683] |
| 8 | 5623 | KMTEKTVKAKSSVPASEDAY | PTTG-1 [92-111] | 25 | 5873 | QRGSKKHLLLAPSDVAGWGI | EZH2 [607- |
| | | | | | | | 626] |
| 8 | 5624 | KMLKEFAKAAIRVQPQDLIQ | Ropporin-1A [16- | 25 | 5874 | TYVPLLGSLRTAQTQLSRKL | hTERT [1088- |
| | | | 35] | | | | 1107] |
| 8 | 5625 | IQEPEIALKRIAASALSDIA | SPAG6 [176-195] | 25 | 5875 | QNLVPFRDSKLTRVFQGFFT | KIF20A [454- |
| | | | | | | | 473] |
| 8 | 5626 | RKLFEVESVTHHDYRMELAQ | SPAG8 [373-392] | 25 | 5876 | KREEKKAQNSEMRMKRAQEY | MCAK [204- |
| | | | | | | | 223] |
| 8 | 5627 | MNGDDAFARRPRAGAQIPEK | SSX-7 [1-20] | 25 | 5877 | PQAPRRPLPQVATLIERFVK | MSLN [409- |
| | | | | | | | 428] |
| 8 | 5628 | RRGQIIRMVTDTLVEVLLYD | TDRD4 [980-999] | 25 | 5878 | LSEEPEDHIKAAAAWALGQI | SPAG6 [344- |
| | | | | | | | 363] |
| 8 | 5629 | KIIRLYDYEITDQYIYMVME | TTK [584-603] | 25 | 5879 | EEFARTTDDYLSNLIKAKSY | TDRD4 [210- |
| | | | | | | | 229] |
| 8 | 5630 | HTENRRGALIPNGESLLKRS | WBP2NL [7-26] | 25 | 5880 | DSRGQTTKARFLYGENMPPQ | TTK [230-249] |
| 8 | 5631 | TTYDPSSGYIIVKVPPDMNS | ZNF645 [318-337] | 26 | 5881 | SEKLQLDLSSINISAKEFEV | ATAD2 [662- |
| | | | | | | | 681] |
| 9 | 5632 | SSSKYAPSYYHVMPKQNSTL | ATAD2 [1114- | 26 | 5882 | RNKKNSRRVSFADTIKVFQT | CASC5 [51-70] |
| | | | 1133] | | | | |
| 9 | 5633 | QLNNRERRNVEFTSSHATAV | CASC5 [1302- | 26 | 5883 | DLPTENQEENLSMEKSHHFE | CCDC110 |
| | | | 1321] | | | | [115-134] |
| 9 | 5634 | MSQADTVILDKSKITVPFLK | CCDC110 [212- | 26 | 5884 | TVYAEPPKPFITSNNSNPVE | CEA [317-336] |
| | | | 231] | | | | |
| 9 | 5635 | QQHTQVLFIAKITPNNNGTY | CEA [634-653] | 26 | 5885 | VVDATRKGNKIRFANHSVNP | EZH2 [674- |
| | | | | | | | 693] |
| 9 | 5636 | VYRQQREVYVKGLLAKIFEL | CEP55 [186-205] | 26 | 5886 | SLLRSHYREVLPLATFVRRL | hTERT [12-31] |
| 9 | 5637 | YQHLEGAKEFAAALTAERIK | EZH2 [325-344] | 26 | 5887 | QGTIKEGGILPRSLALIFNS | KIF20A [171- |
| | | | | | | | 190] |
| 9 | 5638 | IKKASVLLIRKLYILMQDLE | HORMAD2 [153- | 26 | 5888 | IRALGQNKAHTPFRESKLTQ | MCAK [527- |
| | | | 172] | | | | 546 |
| 9 | 5639 | RSLLRSHYREVLPLATFVRR | hTERT [11-30] | 26 | 5889 | LFSLGWVQPSRTLAGETGQE | MSLN [25-44] |
| 9 | 5640 | LNQNSSRSHSIFSIRILHLQ | KIF20A [373-392] | 26 | 5890 | TNTLPVLLSLYMSTESSEDL | SPAG6 [377- |
| | | | | | | | 396] |
| 9 | 5641 | AKHFSALRDVIKALRLAMQL | MCAK [691-710] | 26 | 5891 | ISPEKIYVQWLLTENLLNSL | TDRD4 [926- |
| | | | | | | | 945] |
| 9 | 5642 | RQLDVLYPKARLAFQNMNGS | MSLN [479-498] | 26 | 5892 | ISVKGRIYSILKQIGSGGSS | TTK [518-537] |
| 9 | 5643 | SQVSTPRFGKTFDAPPALPK | PTTG-1 [39-58] | 27 | 5893 | KIDLHKYLTVKDYLRDIDLI | ATAD2 [1037- |
| | | | | | | | 1056] |
| 9 | 5644 | PELLKILHSQVAGRLIIRAE | Ropporin-1A [69- | 27 | 5894 | QDLIKDPRNLLANQTLVYSQ | CASC5 [1415- |
| | | | 88] | | | | 1434] |
| 9 | 5645 | LKDKDEYVKKNASTLIREIA | SPAG6 [260-279] | 27 | 5895 | IKNAKSEASIYKNSLSEIGK | CCDC110 |
| | | | | | | | [680-699] |
| 9 | 5646 | FEVESVTHHDYRMELAQAGT | SPAG8 [376-395] | 27 | 5896 | ETQNPVSARRSDSVILNVLY | CEA [216-235] |
| 9 | 5647 | DIAKYFSKKEWEKMKSLEKI | SSX-7 [27-46] | 27 | 5897 | IFAKRAIQTGEELFFDYRYS | EZH2 [710- |
| | | | | | | | 729] |
| 9 | 5648 | RSHFEKNTTLHYHPPILPKE | TDRD4 [657-676] | 27 | 5898 | TDALRGSGAWGLLLRRVGDD | hTERT [128- |
| | | | | | | | 147] |
| 9 | 5649 | IGSGGSSKVFQVLNEKKQIY | TTK [531-550] | 27 | 5899 | PVPANIRFSIWISFFEIYNE | KIF20A [292- |
| | | | | | | | 311] |
| 9 | 5650 | GGWEGQATFKLVFRNGDAIE | WBP2NL [105-124] | 27 | 5900 | ETGQEAAPLEGVLANPPNIS | MSLN [40-59] |
| 9 | 5651 | SASEFASHHYNLNILPQFTE | ZNF645 [351-370] | 27 | 5901 | YYSPGYSDTLLQRVDSYQPL | SPAG6 [488- |
| | | | | | | | 507] |
| 10 | 5652 | QEEDTFRELRIFLRNVTHRL | ATAD2 [981-1000] | 27 | 5902 | YDDGLWYRAKIVAIKEFNPL | TDRD4 [1490- |
| | | | | | | | 1509] |
| 10 | 5653 | RPMEKTVVFVENHVELEMTE | CASC5 [963-982] | 27 | 5903 | QVLNEKKQIYAIKYVNLEEA | TTK [541-560] |
| 10 | 5654 | LQNVSMVQSEISEILNKSII | CCDC110 [74-93] | 28 | 5904 | SLDLSSDFLSLEHIGRRRLR | ATAD2 [21-40] |
| 10 | 5655 | PPAQYSWLIDGNIQQHTQEL | CEA [443-462] | 28 | 5905 | FQDLSINSADKIHITRSHIM | CASC5 [398- |
| | | | | | | | 417] |
| 10 | 5656 | EVHNLNQLLYSQRRADVQHL | CEP55 [275-294] | 28 | 5906 | SKMKPLIFTTQSLIQKVETY | CCDC110 |
| | | | | | | | [457-476] |
| 10 | 5657 | EWKQRRIQPVHILTSVSSLR | EZH2 [59-78] | 28 | 5907 | TYLWWVNGQSLPVSPRLQLS | CEA [531-550] |
| 10 | 5658 | KRYLRMAVLTLYTDPMGSEK | HORMAD2 [96- | 28 | 5908 | YRYSQADALKYVGIEREMEI | EZH2 [726- |
| | | | 115] | | | | 745] |
| 10 | 5659 | GLLLDTRTLEVQSDYSSYAR | hTERT [932-951] | 28 | 5909 | SVWSKLQSIGIRQHLKRVQL | hTERT [579- |
| | | | | | | | 598] |
| 10 | 5660 | TVKEMVKDVLKGQNWLIYTY | KIF20A [140-159] | 28 | 5910 | FRDSKLTRVFQGFFTGRGRS | KIF20A [459- |
| | | | | | | | 478] |
| 10 | 5661 | EELSSQMSSFNEAMTQIREL | MCAK [625-644] | 28 | 5911 | VIQHLGYLFLKMSPEDIRKW | MSLN [368- |
| | | | | | | | 387] |
| 10 | 5662 | QGGIPNGYLVLDLSMQEALS | MSLN [587-606] | 28 | 5912 | AVSMKVFREEDGVLIVELQK | TDRD4 [606- |
| | | | | | | | 625] |
| 10 | 5663 | AHLPLSGVPLMILDEERELE | PTTG-1 [137-156] | 28 | 5913 | LNKLIGRYSQAIEALPPDKY | TTK [84-103] |
| 10 | 5664 | GPDGIITVNEFTQNPRVQLE | Ropporin-1A [193- | 29 | 5914 | RIRSRYSGVNQSMLFDKLIT | ATAD2 [159- |
| | | | 212] | | | | 178] |
| 10 | 5665 | LPHDSKARRLFVTSGGLKKV | SPAG6 [440-459] | 29 | 5915 | EKLQDGRITIREFFILLQVH | CASC5 [1887- |
| | | | | | | | 1906] |
| 10 | 5666 | SYQPPGNVYWPLRGKREAML | SPAG8 [334-353] | 29 | 5916 | IAESENQIQPQSALKVLQQQ | CCDC110 [43- |
| | | | | | | | 62] |
| 10 | 5667 | SSEMPVSLRDALVFMELAKF | TDRD4 [632-651] | 29 | 5917 | NGIPQQHTQVLFIAKITPNN | CEA [630-649] |
| 10 | 5668 | KAVERGAVPLEMLEIALRNL | TTK [168-187] | 29 | 5918 | FSVLNYERARRPGLLGASVL | hTERT [662- |
| | | | | | | | 681] |
| 10 | 5669 | WEGQATFKLVFRNGDAIEFA | WBP2NL [107-126] | 29 | 5919 | PRSLALIFNSLQGQLHPTPD | KIF20A [181- |
| | | | | | | | 200] |
| 10 | 5670 | PQFTQTDAMEHRRWPAWKRL | ZNF645 [377-396] | 29 | 5920 | SLSPEELSSVPPSSIWAVRP | MSLN [451- |
| | | | | | | | 470] |
| 11 | 5671 | AVLQKMDDMKKMRRQRMREL | ATAD2 [183-202] | 29 | 5921 | QDKNQWRRGQIIRMVTDTLV | TDRD4 [974- |
| | | | | | | | 993] |
| 11 | 5672 | EKSTKIDTTSFLANLKLHTE | CASC5 [198-217] | 29 | 5922 | ITDSITLKNKTESSLLAKLE | TTK [350-369] |
| 11 | 5673 | LKNYNNFYRFLPTAPPNVMS | CCDC110 [194- | 30 | 5923 | IYKDRMKIGASLADVDPMQL | ATAD2 [400- |
| | | | 213] | | | | 419] |
| 11 | 5674 | NGNRTLTLFNVTRNDTASYK | CEA [195-214] | 30 | 5924 | RLLGEEIEYLKRWGPNYNLM | CASC5 [2237- |
| | | | | | | | 2256] |
| 11 | 5675 | FNSSINNIHEMEIQLKDALE | CEP55 [160-179] | 30 | 5925 | LHSVEEKLSGDSVNSLPQSV | CCDC110 |
| | | | | | | | [139-158] |
| 11 | 5676 | NGEHRIGIFAKRAIQTGEEL | EZH2 [703-722] | 30 | 5926 | VHNLPQHLFGYSWYKGERVD | CEA [55-74] |
| 11 | 5677 | ITNEHESLKMVKKLFATSIS | HORMAD2 [25-44] | 30 | 5927 | AFKSHVSTLTDLQPYMRQFV | hTERT [758- |
| | | | | | | | 777] |
| 11 | 5678 | DKEQLRPSFLLSSLRPSLTG | hTERT [337-356] | 30 | 5928 | RILRSRRSPLLKSGPFGKKY | KIF20A [871- |
| | | | | | | | 890] |
| 11 | 5679 | SELALRRSQRLAASASTQQL | KIF20A [670-689] | 30 | 5929 | RITKANVELLPRGAPERQRL | MSLN [143- |
| | | | | | | | 162] |
| 11 | 5680 | ITAQENDMEVELPAAANSRK | MCAK [122-141] | 30 | 5930 | VGDEGTIFVVPKLSEFELIK | TDRD4 [1190- |
| | | | | | | | 1209] |
| 11 | 5681 | FRLDALPLDLLLFLNPDAFS | MSLN [113-132] | 30 | 5931 | VQNSHFKEPLVSDEKSSELI | TTK [330-349] |

### Example 17—Personalised Selection of Peptides to Treat Cancer

Effective immunotherapy for cancer patients stimulates T cell responses (ideally CD4+ and CD8+ T cell responses) that target TAA expressed by the cancer cells of the specific patient.

For treatment of a specific cancer patient, peptides comprising one or more of the 3286 hotspot amino acid sequences can be selected based on (i) cancer type (select peptides comprising hotspot sequences that are fragments of TSAs that are associated with the patient's cancer); (ii) TSA expression or TSA expression rate (sample patient's cancer cells and select peptides comprising hotspot sequences that are fragments of a TSA in fact expressed in the patient's cancer cells; or select peptides comprising hotspot sequences that are fragments of TSAs that are most frequently expressed in the patient's type of cancer); (iii) patient HLA genotype (select peptides comprising fragments of TSAs that comprise an amino acid sequence that is a T cell epitope capable of binding to at least three HLA class I alleles of the patient (HLA class I-binding PEPI3+) (and ideally also comprise an amino acid sequence that is a T cell epitope capable of binding to multiple HLA class II alleles of the patient (HLA class II-binding PEPI2/3/4/5+)).

To illustrate the options for selecting peptides comprising hotspot amino acid sequences to treat individual patients, HLA class I-binding PEPI3+ were identified in the hotspot sequences for three colorectal cancer patients, one ovarian cancer patient and three breast cancer patients with known HLA genotypes. Only hotspot sequences that are fragments of TSA associated with the corresponding cancer (colorectal, ovarian, breast, Table 24) were considered. The number of hotspots sequences containing such a PEPI3+ and the number of antigens that could be targeted using selected peptides comprising a hotspot amino acid sequence are shown in Table 26. As expected, more hotspots containing PEPI3+ were identified and more TSA could be targeted using the hotspot sequences identified following a greater number of cycles of the method described in Example 16.

TABLE 26
Number of selectable peptides matching to the patient HLA background) from antigens
related to the indication. In case of MaxCycle = 1, each antigen has only one potential
peptide, therefore the peptide and antigen are selected together. The MaxCycle >1 means
that more than one peptides are related to a single antigen and hopefully more than one is
matching to the patient's HLA set, therefore #peptides >= #antigens.

| Indication | | MaxCycle = 1 | | MaxCycle = 3 | | MaxCycle = 5 | | MaxCycle = 30 | |
|---|---|---|---|---|---|---|---|---|---|
| max(#AG) | Patient ID | #Peptides | #AG | #Peptides | #AG | #Peptides | #AG | #Peptides | #AG |
| Colorectal | CRC_P1 | 16 | 16 | 32 | 24 | 36 | 25 | 63 | 27 |
| (70 AGs) | CRC_P2 | 13 | 13 | 29 | 15 | 34 | 27 | 54 | 28 |
| | CRC_P3 | 14 | 14 | 36 | 31 | 48 | 37 | 98 | 43 |
| Ovarian | OC_P1 | 10 | 10 | 17 | 14 | 28 | 20 | 51 | 22 |
| (54 AGs) | | | | | | | | | |
| Breast | BRC_P1 | 27 | 17 | 45 | 31 | 60 | 34 | 99 | 35 |
| (70 AGs) | BRC_P2 | 23 | 13 | 43 | 35 | 48 | 36 | 86 | 36 |
| | BRC_P3 | 13 | 13 | 26 | 19 | 33 | 23 | 60 | 28 |

Table 27 shows the peptide/hotspot amino acid sequences that are fragments of the breast cancer-associated TSA identified just in the first cycle of the method described in Example 16 that are expected to induce T cell responses in the three breast cancer patients of Table 26.

TABLE 27
Options for peptide selection for breast cancer patients. Sequences are
fragments of known breast cancer-associated CTAs and were identified in first
cycle of method described in Example 16. “+” indicates peptide comprises
subject-matched HLA class I-binding PEPI3]

| SEQ ID NO | Sequence | BRC P1 | BRC P2 | BRC P3 |
|---|---|---|---|---|
| 4 | PHNFRVYSYSGTGIMKPLDQ | - | - | - |
| 7 | DEVSFYANRLTNLVIAMARK | + | + | - |
| 8 | IDDLSFYVNRLSSLVIQMAH | - | + | + |
| 10 | ARAVFLALSAQLLQARLMKE | - | - | - |
| 16 | QTTQNGRFYAISARFKPFSN | - | - | - |
| 19 | EGKDPAFTALLTTQLQVQRE | - | - | - |
| 20 | QGPTAVRKRFFESIIKEAAR | - | - | - |
| 21 | ATAQLQRTPMSALVFPNKIS | - | + | - |
| 22 | LDMVHSLLHRLSHNDHILIE | + | - | - |
| 26 | HTRFTQSGTMKIHILQKHGE | + | - | - |
| 29 | NTGEMSSNSTALALVRPSSS | - | - | - |
| 32 | NLLRGIDSLFSAPMDFRGLP | - | + | - |
| 35 | SERVRTYWIIIELKHKAREK | + | + | - |
| 40 | EHLEVKFMNPYNAVLTKKFQ | + | + | - |
| 41 | SGSSYFVLANGHILPNSENA | - | - | - |
| 42 | ISDTKDYFMSKTLGIGRLKR | + | - | - |
| 44 | EEMRSHYVAQTGILWLLMNN | - | - | - |
| 45 | SRGKSTYYWPRPRRYVQPPE | - | - | - |
| 46 | WRGRSTYYWPRPRRYVQPPE | - | - | - |
| 48 | ALSRHMSSLSHISPFSHSSH | - | + | - |
| 51 | RLNDLQMSNFVNLKNITYLV | - | + | - |
| 54 | KKVNFLDMSLDDIIIYKELE | - | - | + |
| 57 | KLRYRYTLDDLYPMMNALKL | + | + | + |
| 60 | PDSRLLQLHITMPFSSPMEA | + | + | - |
| 61 | QRNVAIMKSIIPAIVHYSPD | + | - | - |
| 62 | SEELNIILQGNIILSTEKSK | - | - | - |
| 64 | VKVLEYVIKVSARVRFFFPS | - | + | + |
| 65 | DDTTAMASASSSATGSFSYP | - | - | - |
| 66 | DPTSHSYVLVTSLNLSYDGI | + | + | - |
| 67 | ALSRKMAELVHFLLLKYRAR | + | + | - |
| 68 | GREDSVFAHPRKLLMQDLVQ | + | - | + |
| 69 | FPVIFSKASSSLQLVFGIEL | - | - | - |
| 70 | VNARVRIAYPSLREAALLEE | - | - | - |
| 71 | KSPQGASAIPTAIDFTLWRQ | - | - | - |
| 72 | KKLLTQYFVQENYLEYRQVP | - | - | + |
| 73 | RAPEEAIWEALSVMGLYDGR | - | + | - |
| 74 | PAQLEFMFQEALKLKVAELV | + | + | + |
| 80 | RPVSSFFSYTLASLLQSSHE | - | - | + |
| 81 | ATVMASESLSVMSSNVSFSE | + | - | - |
| 83 | RSQAGMFIYSNNRLIKMHEK | + | - | + |
| 87 | EEAFSRASLVSVYNSYPYYP | + | - | - |
| 90 | QSQGSVLAFTRTFIATPGSS | - | + | - |
| 91 | KRASQYSGQLKVLIAENTML | + | - | - |
| 93 | KEFTVSGNILTIRLTAADHR | - | - | - |
| 96 | EKSTKYVFDSAPSHSISART | - | - | - |
| 97 | NSPLPFQWRITHSFRWMAQV | - | + | + |
| 105 | KEMELWMKAMLDAALVQTEP | + | - | - |
| 106 | QYAHKLAFLVGQSIHREPNL | + | - | - |
| 107 | RSVVGFVASINLTLTKWYSR | + | + | + |
| 110 | KAVSQDMVIYSTEIHYSSKG | - | - | - |
| 111 | RLDQLLRHVMNPLETLSITN | - | + | - |
| 116 | PPAFINMAATGVSSMSTRDQ | + | - | - |
| 118 | NNNIEKMITAFEELRVQAEN | - | + | - |
| 122 | QSSGISFQSKYLSFFILGQT | - | - | - |
| 124 | PDNIPAFAAAYFESLLEKRE | + | + | - |
| 126 | RDAVKFFVAVPGQVISPQSS | + | - | - |
| 127 | KKMKTSESSTILVVRYRRNV | - | - | + |
| 129 | AEIQALTFLSSDYLSRVYLP | - | - | - |
| 130 | RIQVEHPQMTFGRLHRIIPK | + | - | + |
| 131 | VERPQMTFGRLQGISPKIMP | + | - | - |
| 132 | QRPQMTFGRLQGIFPKIMPK | + | - | - |
| 133 | SEKIVYVYMKLNYEVMTKLG | - | + | - |
| 134 | NQVEHPQMTFGRLQGIFPKI | - | - | - |
| 135 | EFEETAKKVRRAIEQLAAMD | - | - | - |
| 142 | LSQFWEFSETTASTVSTTLH | - | - | - |
| 149 | EELQKVQFEKVSALADLSST | + | + | - |
| 155 | VGILHLGSRQKKIRIQLRSQ | - | - | - |

### Example 18—Selection of Polynucleic Acids for Treatment of Cancer

Some peptides that are difficult to manufacture and use as peptide vaccines can still be used in vaccines and immunotherapy if delivered to patients as peptide-encoding polynucleic acids or vectors. To optimally design a set of peptides for treating cancer to be encoded by a nucleic acid or vector for administration to patients, the method of Example 16 was repeated but without eliminating hotspot sequences that did not meet the peptide manufacturing feasibility requirements. Table 28 shows the hotspot sequences identified in the first 20 cycles and the TSA of which they are a fragment.

TABLE 28
Hotspot Sequences and corresponding TAA

| | SEQ | | Source Antigen(s) | | SEQ | | Source Antigen(s) |
|---|---|---|---|---|---|---|---|
| | ID | | [AA position | | ID | | [AA position |
| Cycle | NO | Sequence | in Antigen] | Cycle | NO | Sequence | in Antigen] |
| 1 | 2787 | AGDGRLRLARLALVLLGWVS | 5T4 [11-30] | 19 | 4110 | PFKCSMCKYASVEASKLKRH | CTCFL [341- |
| | | | | | | | 360] |
| 1 | 2788 | ELLHTFLRWKTSYLVLRPHD | ADAM2 [245- | 19 | 4111 | DSLCPQVINAALTLAARPQS | CTNNA2 [456- |
| | | | 264] | | | | 475] |
| 1 | 2789 | NKTLGTFSIAVAHHLGHNLG | ADAM29 [320- | 19 | 4112 | FYEHIITVGTGQGSLLFYDI | DCAF12 [348- |
| | | | 339] | | | | 367] |
| 1 | 2790 | IARRHPFLYAPTILLWAARY | AFP [166- | 19 | 4113 | HGRLLSADTKGWVRLQFHAG | DPPA2 [227- |
| | | | 185] | | | | 246] |
| 1 | 2791 | MAARAVFLALSAQLLQARLM | BAGE-1 [1- | 19 | 4114 | NRQCQCTSVGAQNTVICSKL | EpCAM [43- |
| | | | 20] | | | | 62] |
| 1 | 2792 | MAAGVVFLALSAQLLQARLM | BAGE-2 [1- | 19 | 4115 | CSPGFFKFEASESPCLECPE | EPHA2 [276- |
| | | | 20]; BAGE- | | | | 295] |
| | | | 3 [1-20] | | | | |
| 1 | 2793 | MAAGAVFLALSAQLLQARLM | BAGE-5 [1- | 19 | 4116 | GDFQEAMTHLQHKLICTRKP | FAM46D [209- |
| | | | 20] | | | | 228] |
| 1 | 2794 | YKDAYKFAADVRLMFMNCYK | BRDT [331- | 19 | 4117 | PQDQSCWAFLPDLCLCRVFW | FBXO39 [10- |
| | | | 350] | | | | 29] |
| 1 | 2795 | ASQLASKMHSLLALMVGLLT | CAGE1 [610- | 19 | 4118 | EQLKCLLDECILKQKSIIKL | FSIP1 [406- |
| | | | 629] | | | | 425] |
| 1 | 2796 | WAICMLRVALATVYFQEEFL | CALR3 [9-28] | 19 | 4119 | HEIFNLLSFTLNPLEGKLQQ | GASZ [228- |
| | | | | | | | 247] |
| 1 | 2797 | PEVLHMIYMRALQIVYGIRL | CDCA1 [42- | 19 | 4120 | FSHSSHMLTTPTPMHPPSSL | GATA-3 [410- |
| | | | 61] | | | | 429] |
| 1 | 2798 | PCEYYFRVYHSLCPISWVES | COX6B2 [55- | 19 | 4121 | SSRRRELIQKLKSFISFYSA | Glypican- |
| | | | 74] | | | | 3 [385-404] |
| 1 | 2799 | EAARCMRRDFVKHLKKKLKR | CT45 [168- | 19 | 4122 | LHDPSGYLAEADLSYTWDFG | gp100 [244- |
| | | | 187] | | | | 263] |
| 1 | 2800 | LIRKIYILMQNLGPLPNDVC | CT46 [153- | 19 | 4123 | CYVNDIVLAILELLKYHQRV | HDAC1 [151- |
| | | | 172] | | | | 170] |
| 1 | 2801 | HFVLLDMVHSLLHRLSHNDH | CT47 [120- | 19 | 4124 | YGAGHPMKPHRLALTHSLVL | HDAC3 [18- |
| | | | 139] | | | | 37] |
| 1 | 2802 | NYIDQFLLTSFPTFTSVGVL | CTAGE1 [34- | 19 | 4125 | IGTGAVIIIFCIALILGAFF | IGFS11 [247- |
| | | | 53] | | | | 266] |
| 1 | 2803 | FFAVLFLWRSFRSVTSRLYV | CTAGE2 [23- | 19 | 4126 | CTNGSEVQSSWAETTYWISP | IL13RA2 [113- |
| | | | 42] | | | | 132] |
| 1 | 2804 | HCDVCMFTSSRMSSFNRHMK | CTCFL [258- | 19 | 4127 | MPKTGFLIIVLVMIAMEGGH | MAGE- |
| | | | 277] | | | | A1 [189-208] |
| 1 | 2805 | MNFYLLLASSILCALIVFWK | CXorf61 [1- | 19 | 4128 | NMMGLYDGMEHLIYGEPRKL | MAGE-A10 [251-270] |
| | | | 20] | | | | |
| 1 | 2806 | AATVPATAAGVVAVVVPVPA | DBPC [15-34] | 19 | 4129 | LGLVGAQALQAEEQEAAFFS | MAGE- |
| | | | | | | | A11 [133-152] |
| 1 | 2807 | WVPTTHRRMISLFLLPACIF | DPPA2 [249- | 19 | 4130 | FTKAEMLGSVIRNFQDFFPV | MAGE-A12 [130-149] |
| | | | 268] | | | | |
| 1 | 2808 | CSERVRTYWIIIELKHKARE | EpCAM [135- | 19 | 4131 | LLGDNQVMPKTGLLIIVLAI | MAGE- |
| | | | 154] | | | | A2 [189-208] |
| 1 | 2809 | QYTEHFMAAGYTAIEKVVQM | EPHA2 [920- | 19 | 4132 | LVHFLLLKYRAREPVTKAEM | MAGE- |
| | | | 939] | | | | A3 [116-135] |
| 1 | 2810 | YQPAPYFAAEARYPIYVIPE | FAM46D [349- | 19 | 4133 | LLGDNQIMPKTGFLIIILAI | MAGE- |
| | | | 368] | | | | A6 [189-208] |
| 1 | 2811 | ETLIIAVLVSASIANLWLWM | FATE1 [162- | 19 | 4134 | HAETSYEKVINYLVMLNARE | MAGE- |
| | | | 181] | | | | A9 [277-296] |
| 1 | 2812 | CSGSSYFVLANGHILPNSEN | FMR1NB [91- | 19 | 4135 | DPPRFQFLWGPRAYAETSKM | MAGE- |
| | | | 110] | | | | B2 [267-286] |
| 1 | 2813 | TLELYTSYLYLSMAFYFNRD | FTHL17 [26- | 19 | 4136 | DGNQSSAWTLPRNGLLMPLL | MAGE- |
| | | | 45] | | | | B4 [186-205] |
| 1 | 2814 | TGILWLLMNNCFLNLSPRKP | GAGE-1 [119- | 19 | 4137 | SILKADMLKCVRREYKPYFP | MAGE- |
| | | | 138] | | | | B6 [215-234] |
| 1 | 2815 | SVDSNFQYGWTPLMYAASVA | GASZ [71-90] | 19 | 4138 | DYFPVILKRAREFMELLFGL | MAGE- |
| | | | | | | | C2 [176-195] |
| 1 | 2816 | FEFVGEFFTDVSLYILGSDI | Glypican- | 19 | 4139 | GSAATWGQDVTSVPVTRPAL | MUC-1 [89- |
| | | | 3 [142-161] | | | | 108] |
| 1 | 2817 | GKTLCYLMPGFIHLVLQPSL | HAGE [291- | 19 | 4140 | NVSCNQLDTGVPLLCEALCS | NLRP4 [757- |
| | | | 310] | | | | 776] |
| 1 | 2818 | AKCVEFVKSFNLPMLMLGGG | HDAC1 [282- | 19 | 4141 | PLGTPMLIEDGYAVTQAELF | NR6A1 [265- |
| | | | 301] | | | | 284] |
| 1 | 2819 | EASGFCYVNDIVLAILELLK | HDAC2 [147- | 19 | 4142 | AVTCGFHHIHEQIMEYIRKL | NY-BR- |
| | | | 166] | | | | 1 [189-208] |
| 1 | 2820 | GECVEYVKSFNIPLLVLGGG | HDAC3 [277- | 19 | 4143 | AEMDGAAAAKQVMALKDTIG | ODF2 [214- |
| | | | 296] | | | | 233] |
| 1 | 2821 | ALINLNVIWMVTPLSNANQP | IGFS11 [51- | 19 | 4144 | PMVMGPNTVGKASQPSFSIK | ODF3 [145- |
| | | | 70] | | | | 164] |
| 1 | 2822 | GYLGYLYLQWQPPLSLDHFK | IL13RA2 [44- | 19 | 4145 | AVFQCAQCHAVLADSVHLAW | OIP5 [76-95] |
| | | | 63] | | | | |
| 1 | 2823 | CDDSYHTFCLIPPLHDVPKG | JARID1B [330- | 19 | 4146 | GFMITLSTDGVIICVAENIS | PASD1 [43- |
| | | | 349] | | | | 62] |
| 1 | 2824 | DDAAPRVEGVPVAVHKHALH | KAAG1 [3-22] | 19 | 4147 | ADIFAFGLTLWEMMTLSIPH | PBK [228-247] |
| 1 | 2825 | CPDESWALKAIEALSGKIEL | KOC1 [44-63] | 19 | 4148 | TFKLCHMYWNWPGTIRVPAP | PIWIL2 [924- |
| | | | | | | | 943] |
| 1 | 2826 | GIFIIVVFVYLTVENKSLFG | LEMD1 [162- | 19 | 4149 | NNFTLAFIVVKKRINTRFFL | PIWIL3 [752- |
| | | | 181] | | | | 771] |
| 1 | 2827 | KYSAHAFQFSPRNVLWLLVV | LIPI [13-32] | 19 | 4150 | IPQHKLSLWPGFAISVSYFE | PIWIL4 [235- |
| | | | | | | | 254] |
| 1 | 2828 | VDPTGHSFVLVTSLGLTYDG | MAGE- | 19 | 4151 | FDECGITDDQLLALLPSLSH | PRAME [385- |
| | | | A10 [194-] | | | | 404] |
| | | | 213] | | | | |
| 1 | 2829 | VALSRKMAELVHFLLLKYRA | MAGE- | 19 | 4152 | AMNFDFPFKKGSGIPLLTTN | RAGE-1 [234- |
| | | | A12 [107- | | | | 253] |
| | | | 126] | | | | |
| 1 | 2830 | FRAALSKKVADLIHFLLLKY | MAGE- | 19 | 4153 | LEEDLQIATKTICQLTEEKE | SCP-1 [341- |
| | | | A5 [105-124] | | | | 360] |
| 1 | 2831 | VKDPVAWEAGMLMHFILRKY | MAGE- | 19 | 4154 | SCHSFQSAYLIVDRDEAWVL | SCRN1 [149- |
| | | | B1 [104-123] | | | | 168] |
| 1 | 2832 | WTLPRNGLLMPLLSVIFLNG | MAGE- | 19 | 4155 | CCSLDLLMKKIKGKDLQLLE | se57-1 [85- |
| | | | B4 [193-212] | | | | 104] |
| 1 | 2833 | FLWGPRAYAETTKMRVLRVL | MAGE- | 19 | 4156 | KATEYLVIIGASEFLPIYLE | SLCO6A1 [392- |
| | | | B6 [357-376] | | | | 411] |
| 1 | 2834 | RRRNGYRALMDKSLHVGTQC | MART1 [49- | 19 | 4157 | GSDQHVASHLPLHPIMHNKP | SOX-6 [32- |
| | | | 68] | | | | 51] |
| 1 | 2835 | PARWSFRAYTSVLYFNPWMR | MORC1 [232- | 19 | 4158 | GDTFLLLIQSLKNNLIEKDP | SPAG1 [847- |
| | | | 251] | | | | 866] |
| 1 | 2836 | VFCLTVVCWTWVPLRGPSSP | NA17-A [12- | 19 | 4159 | KSDSPKSAQKFSLILKILSM | SPO11 [101- |
| | | | 31] | | | | 120] |
| 1 | 2837 | NQKICIFVNHSTAPYSVKNK | NXF2 [189- | 19 | 4160 | NQEKLCMLTAELLEYCNAPK | TDRD1 [965- |
| | | | 208] | | | | 984] |
| 1 | 2838 | NTALHYAVYSEILSVVAKLL | NY-BR-1 [84- | 19 | 4161 | PVWGLQLLLPLLLPSFIHFS | TEX101 [230- |
| | | | 103] | | | | 249] |
| 1 | 2839 | MVILTILAIAPSVPFAANCF | NYC- | 19 | 4162 | LKRLSSFIGAGSPSLVKACD | TEX14 [1285- |
| | | | TSPG [348- | | | | 1304] |
| | | | 367] | | | | |
| 1 | 2840 | SRLLEFYLAMPFATPMEAEL | NY-ESO- | 19 | 4163 | LSKPIFCFVKDVHPDLEMND | TEX15 [2511- |
| | | | 1 [85-104] | | | | 2530] |
| 1 | 2841 | CLCDYKLYCLRPSLRSLERK | ODF1 [74-93] | 19 | 4164 | SRAAQMAVPSSRILQLSKPK | THEG [256- |
| | | | | | | | 275] |
| 1 | 2842 | LRAPAYSFRGAPMLLAENCS | ODF3 [46-65] | 19 | 4165 | IALQEKESEIQLLKEHLCLA | TSGA10 [583- |
| | | | | | | | 602] |
| 1 | 2843 | IPVGFHLYSTHAALAALRGH | OIP5 [142- | 19 | 4166 | AGCQKSEAPPIYLQVSSYQH | TSP50 [332- |
| | | | 161] | | | | 351] |
| 1 | 2844 | FPTYDYFNQVTLQLLDGFMI | PASD1 [27- | 19 | 4167 | ALLAGLVSLLCRHKRKQLPE | TYR [490-509] |
| | | | 46] | | | | |
| 1 | 2845 | ASQDPFPAAIILKVALNMAR | PBK [133- | 19 | 4168 | PVETKAHFDSSEPQLLWDCD | ZNF165 [165- |
| | | | 152] | | | | 184] |
| 1 | 2846 | EMELWMKAMLDAALVQTEPV | PEPP2 [255- | 20 | 4169 | PILPPSLQTSYVFLGIVLAL | 5T4 [346-365] |
| | | | 274] | | | | |
| 1 | 2847 | PAPCQYAHKLAFLVGQSIHR | PIWIL1 [829- | 20 | 4170 | FESFCQFTHYRCSNHVYYAK | ACRBP [100- |
| | | | 848] | | | | 119] |
| 1 | 2848 | MRSVVGFVASINLTLTKWYS | PIWIL2 [754- | 20 | 4171 | CDRRCLFQLETVAVTQMNKC | ACTL8 [193- |
| | | | 773] | | | | 212] |
| 1 | 2849 | PAPCHYAHKLAYLVGQSIHQ | PIWIL3 [850- | 20 | 4172 | KMCDANYAGGVVLHPRTISL | ADAM2 [285- |
| | | | 869] | | | | 304] |
| 1 | 2850 | LNKQGMMMSIATKIAMQMTC | PIWIL4 [582- | 20 | 4173 | CSQPRCIMHEGNPPITKFSN | ADAM29 [349- |
| | | | 601] | | | | 368] |
| 1 | 2851 | LCSIDWFMVTVHPFMLNNDV | PLAC1 [29- | 20 | 4174 | TLHRNEYGIASILDSYQCTA | AFP [20-39] |
| | | | 48] | | | | |
| 1 | 2852 | HLERLAYLHARLRELLCELG | PRAME [456- | 20 | 4175 | DILCPKAKRTSRFLSGIINF | CDCA1 [108- |
| | | | 475] | | | | 127] |
| 1 | 2853 | NASGAHALQPAAAILALLPA | PSCA [84- | 20 | 4176 | APPWEQDYRMMFPPPGQSYP | CTAGE2 [565- |
| | | | 103] | | | | 584] |
| 1 | 2854 | EKKIMHYMYQLCKSLDHIHR | RAGE-1 [102- | 20 | 4177 | KGAKGTFHCDVCMFTSSRMS | CTCFL [251- |
| | | | 21] | | | | 270] |
| 1 | 2855 | PTGLINMAATPIPAMSARDL | SAGE1 [179- | 20 | 4178 | PHCRDEMAAARGALKKNATM | CTNNA2 [200- |
| | | | 198] | | | | 219] |
| 1 | 2856 | KRCQHKIAEMVALMEKHKHQ | SCP-1 [701- | 20 | 4179 | TKCNTLFVVDVQTSQITKIP | DCAF12 [107- |
| | | | 720] | | | | 126] |
| 1 | 2857 | QVPRTYAIMISRPAWLWGAE | SCRN1 [61- | 20 | 4180 | KVCRDTLRDWCQQLGLSTNG | DPPA2 [94- |
| | | | 80] | | | | 113] |
| 1 | 2858 | YKLPLFIAFIFSTLIFSGFS | SLCO6A1 [576- | 20 | 4181 | SKLAAKCLVMKAEMNGSKLG | EpCAM [60- |
| | | | 595] | | | | 79] |
| 1 | 2859 | EEAVMYYTRSISALPTVVAY | SPAG1 [226- | 20 | 4182 | ERYMCSRFFIDFPHIEEQQK | FAM46D [259- |
| | | | 245] | | | | 278] |
| 1 | 2860 | TGKLGFSFVRITALMVSCNR | SPAG9 [1138- | 20 | 4183 | WASPQNFTSVCEELVNNVED | GASZ [392- |
| | | | 1157] | | | | 411] |
| 1 | 2861 | VEHPQMTFGRLHRIIPKIMP | SSX-1 [94- | 20 | 4184 | IDKYWREYILSLEELVNGMY | Glypican- |
| | | | 113] | | | | 3 [292-311] |
| 1 | 2862 | EKIVYVYMKLNYEVMTKLGF | SSX-4 [44- | 20 | 4185 | GTATLRLVKRQVPLDCVLYR | gp100 [460- |
| | | | 63] | | | | 479] |
| 1 | 2863 | EHPQMTFGRLQGIFPKITPE | SSX-5 [95- | 20 | 4186 | HYGLYKKMIVFKPYQASQHD | HDAC3 [38- |
| | | | 114] | | | | 57] |
| 1 | 2864 | PHPGTVISHSVAMLILIGSL | TAG-1 [1019- | 20 | 4187 | GRQCVEHYRLLHRYCVFSHD | JARID1B [611- |
| | | | 1038] | | | | 630] |
| 1 | 2865 | LPASTLSRLSNRLLLRLECN | TAG-2a [37- | 20 | 4188 | TGAVGMACAISILLKDLADE | LDHC [28-47] |
| | | | 56] | | | | |
| 1 | 2866 | YLPGYRYLNSWRPSLFYKIA | TEKT5 [40- | 20 | 4189 | GASLDNFHFIGVSLGAHISG | LIPI [168- |
| | | | 59] | | | | 187] |
| 1 | 2867 | AMIGCRLMSGILAVGPMFVR | TEX101 [187- | 20 | 4190 | LESLFRAVITKKVADLVGFL | MAGE-A1 [94- |
| | | | 206] | | | | 113] |
| 1 | 2868 | LTGATQMAYLGSLPVIGEKE | TEX14 [221- | 20 | 4191 | AQIACSSPSVVASLPLDQSD | MAGE- |
| | | | 240] | | | | A10 [87-106] |
| 1 | 2869 | TLYFYKFFLPTILSLSFFIL | TMEM31 [132- | 20 | 4192 | LGDNQIVPKTGLLIIVLAII | MAGE-A12 [190-209] |
| | | | 151] | | | | |
| 1 | 2870 | GFLEKEFYHKTNIKMRCEFL | TRAG-3 [18- | 20 | 4193 | YLQLVFGIEVVEVVPISHLY | MAGE- |
| | | | 37] | | | | A2 [157-176] |
| 1 | 2871 | APSRTLLLALPLPLSLLAAL | TSP50 [366- | 20 | 4194 | IGHLYIFATCLGLSYDGLLG | MAGE- |
| | | | 385] | | | | A3 [172-191] |
| 1 | 2872 | HLCKIMLFFRSNPYFQNKVI | TSPY1 [197- | 20 | 4195 | IWKFMNVLGAYDGEEHLIYG | MAGE- |
| | | | 216] | | | | B1 [220-239] |
| 1 | 2873 | KGAATKMAAVTAPEAESAPA | VCX [53-72] | 20 | 4196 | CATEEKIWEFLNKMRIYDGK | MAGE- |
| | | | | | | | B3 [217-236] |
| 1 | 2874 | DVRDLNALLPAVPSLGGGGG | WT1 [4-23] | 20 | 4197 | RLSKDAVKKKACTLAQFLQK | MAGE- |
| | | | | | | | B6 [189-208] |
| 1 | 2875 | SRQKKIRIQLRSQCATWKVI | XAGE-1 [20- | 20 | 4198 | RYTGYFPVIFRKAREFIEIL | MAGE- |
| | | | 39]; XAGE- | | | | C1 [941-960] |
| | | | 1b [20-39] | | | | |
| 1 | 2876 | CKECGRAFNLNSHLIRHQRI | ZNF165 [402- | 20 | 4199 | ARWSFRAYTSVLYFNPWMRI | MORC1 [233- |
| | | | 421] | | | | 252] |
| 2 | 2877 | PTSSASSFSSSAPFLASAVS | 5T4 [34-53] | 20 | 4200 | QSGAGVPGWGIALLVLVCVL | MUC-1 [1151- |
| | | | | | | | 1170] |
| 2 | 2878 | SVWLGASVVAHLSTYQSEWM | ACTL8 [336- | 20 | 4201 | LVANFEKARRAHWIFLGCFL | NLRP4 [484- |
| | | | 355] | | | | 503] |
| 2 | 2879 | VFQLIGLTNAIFVSFNITII | ADAM2 [205- | 20 | 4202 | QLNKRYWYICQDFTEYKYTH | NR6A1 [413- |
| | | | 224] | | | | 432] |
| 2 | 2880 | ETFMNKFIYEIARRHPFLYA | AFP [156- | 20 | 4203 | DQSAYVSAIRDCFPKLLRLD | NXF2 [338- |
| | | | 175] | | | | 357] |
| 2 | 2881 | SGQMVEHLMNSVMKLCVIIA | AKAP-3 [666- | 20 | 4204 | TRKDCLAKHLKHMRRMYGTS | NYC- |
| | | | 685] | | | | TSPG [154- |
| | | | | | | | 173] |
| 2 | 2882 | AVLQWIAASQFNVPMLYFMG | AKAP-4 [776- | 20 | 4205 | IDTSNSEAISSSSIPQFPIT | PASD1 [656- |
| | | | 795] | | | | 675] |
| 2 | 2883 | RCIIILVLQEPTAFRISVTS | BAGE-2 [60- | 20 | 4206 | KELTPTSPDCLRYYNILFRR | PIWIL3 [212- |
| | | | 79] | | | | 231] |
| 2 | 2884 | SVTSSCFVQNTLTKLLKDRR | BAGE-3 [76- | 20 | 4207 | LETWGLHFGSQISLTGRIVP | PIWIL4 [431- |
| | | | 95]; BAGE- | | | | 450] |
| | | | 2 [76-95] | | | | |
| 2 | 2885 | MLAKKHFSYAWPFYNPVDVN | BRDT [283- | 20 | 4208 | VLPGLAYLATADMPAMSTRD | SAGE1 [366- |
| | | | 302] | | | | 385] |
| 2 | 2886 | CKVDGFTHLYTLILRPDLSY | CALR3 [163- | 20 | 4209 | MIGYALGYVLGAPLVKVPEN | SLCO6A1 [275- |
| | | | 182] | | | | 294] |
| 2 | 2887 | CCHPSNFIIEAPGHMSDVEW | CCDC62 [515- | 20 | 4210 | FVRITALMVSCNRLWVGTGN | SPAG9 [1145- |
| | | | 534] | | | | 1164] |
| 2 | 2888 | IRLEHFYMMPVNSEVMYPHL | CDCA1 [59- | 20 | 4211 | KKLWDTFHVPVFTLVDADPH | SPO11 [262- |
| | | | 78] | | | | 281] |
| 2 | 2889 | NCYQNFLDYHRCLKTRTRRG | COX6B2 [31- | 20 | 4212 | EDVPLAAKLVDLPCVIESLR | TAF7L [145- |
| | | | 50] | | | | 164] |
| 2 | 2890 | VGHYTQLVWYSTYQVGCGIA | CRISP2 [134- | 20 | 4213 | GYVDYGNFEILSLMRLCPII | TDRD1 [575- |
| | | | 153] | | | | 594] |
| 2 | 2891 | PTAVRKRFFESIIKEAARCM | CT45 [154- | 20 | 4214 | QDTLQLLVMTKCRLEHELAI | TEKT5 [437- |
| | | | 173] | | | | 456] |
| 2 | 2892 | HAAARNLWGNLPPLLLPQRL | CT47 [155- | 20 | 4215 | AFCELQTYHDQLVELLEETK | TEX15 [1755- |
| | | | 174] | | | | 1774] |
| 2 | 2893 | MFVIISLHNCVVISFVLFLF | CTAGE1 [1- | 20 | 4216 | IKLLRNIPRWTHLLRLLRLI | TPTE [181- |
| | | | 20] | | | | 200] |
| 2 | 2894 | VRREKKFAVALSGLIEEKCK | CTAGE2 [42- | 20 | 4217 | WVLTVAHCLIWRDVIYSVRV | TSP50 [147- |
| | | | 61] | | | | 166] |
| 2 | 2895 | HICHTRFTQSGTMKIHILQK | CTCFL [401- | 20 | 4218 | STPMFNDINIYDLFVWMHYY | TYR [163-182] |
| | | | 420] | | | | |
| 2 | 2896 | HISDSFLETNVPLLVLIEAA | CTNNA2 [387- | 20 | 4219 | PFMCAYPGCNKRYFKLSHLQ | WT1 [322-341] |
| | | | 406] | | | | |
| 2 | 2897 | MLRLLRLALAFYGRTADPAE | CXorf48 [1- | 20 | 4220 | HDGCERRLNLNSNEFTHQKS | ZNF165 [272- |
| | | | 20] | | | | 291] |
| 2 | 2898 | RWCPPPFFYRRRFVRGPRPP | DBPC [222- | 1 | 4221 | VESTPMIMENIQELIRSAQE | ACRBP [275- |
| | | | 241] | | | | 294] |
| 2 | 2899 | VLAFGLLLAAATATFAAAQE | EpCAM [6-25] | 1 | 4222 | LNWEGVQYLWSFVLENHRRE | ACTL8 [73- |
| | | | | | | | 92] |
| 2 | 2900 | WDLMQNIMNDMPIYMYSVCN | EPHA2 [52- | 1 | 4223 | DEVSFYANRLTNLVIAMARK | AKAP-3 [122- |
| | | | 71] | | | | 141] |
| 2 | 2901 | VPKQMMQMFGLGAISLILVC | FMR1NB [180- | 1 | 4224 | IDDLSFYVNRLSSLVIQMAH | AKAP-4 [214- |
| | | | 199] | | | | 233] |
| 2 | 2902 | ACGLYYKLHNINRPLTMKKE | GATA-3 [341- | 1 | 4225 | RTKKELASALKSALSGHLET | ANXA2 [78- |
| | | | 360] | | | | 97] |
| 2 | 2903 | IQKLKSFISFYSALPGYICS | Glypican- | 1 | 4226 | PSNINQFAAAYFQELTMYRG | CABYR [30- |
| | | | 3 [392-411] | | | | 49] |
| 2 | 2904 | LLVLMAVVLASLIYRRRLMK | gp100 [603- | 1 | 4227 | SPASELIAIQDSHSLGSSKS | CCDC62 [566- |
| | | | 622] | | | | 585] |
| 2 | 2905 | NDLQMSNFVNLKNITYLVLD | HAGE [377- | 1 | 4228 | EGKDPAFTALLTTQLQVQRE | CRISP2 [20- |
| | | | 396] | | | | 39] |
| 2 | 2906 | ASGFCYVNDIVLAILELLKY | HDAC1 [147- | 1 | 4229 | FAVLFFLWRSFRSVRSRLYV | CTAGE5 [53- |
| | | | 166] | | | | 72] |
| 2 | 2907 | GAESGRYYCLNVPLRDGIDD | HDAC3 [210- | 1 | 4230 | KKNATMLYTASQAFLRHPDV | CTNNA2 [214- |
| | | | 229] | | | | 233] |
| 2 | 2908 | TVTIRNISALSSGLYQCVAS | IGFS11 [199- | 1 | 4231 | NEDNIYISNSIYFSIAIVSE | CXorf48 [122- |
| | | | 218] | | | | 141] |
| 2 | 2909 | YKLRYRYTLDDLYPMMNALK | JARID1B [730- | 1 | 4232 | SRVLHGYAAQQLPSLLKERE | DCAF12 [64- |
| | | | 749] | | | | 83] |
| 2 | 2910 | LHDGLRQVAGPGAAAAHLPR | KAAG1 [21- | 1 | 4233 | NLLRGIDSLFSAPMDFRGLP | DKKL1 [63- |
| | | | 40] | | | | 82] |
| 2 | 2911 | GTGAVGMACAISILLKDLAD | LDHC [27-46] | 1 | 4234 | IAERQRVMAAQVALRRQQAQ | DMRT1 [108- |
| | | | | | | | 127] |
| 2 | 2912 | STRKLYEKKLVQLLVSPPCA | LEMD1 [29- | 1 | 4235 | KRDNRVAYMNPIAMARWRGP | FAM133A [3- |
| | | | 48] | | | | 22] |
| 2 | 2913 | DYFPEIFREASVCMQLLFGI | MAGE- | 1 | 4236 | EHLEVKFMNPYNAVLTKKFQ | FBXO39 [90- |
| | | | A11 [258- | | | | 109] |
| | | | 277] | | | | |
| 2 | 2914 | FQDFFPVIFSKASEYLQLVF | MAGE- | 1 | 4237 | ISDTKDYFMSKTLGIGRLKR | FSIP1 [515- |
| | | | A12 [143- | | | | 534] |
| | | | 162] | | | | |
| 2 | 2915 | PPQSPQGASALPTTISFTCW | MAGE-A4 [62- | 1 | 4238 | MSWRGRSTYRPRPRRYVEPP | GAGE-2 [1- |
| | | | 81] | | | | 20]; GAGE- |
| | | | | | | | 1 [1-20]; |
| | | | | | | | GAGE-8 [1- |
| | | | | | | | 20] |
| 2 | 2916 | LKSPQGASAIPTAIDFTLWR | MAGE-A5 [62- | 1 | 4239 | SRGKSTYYWPRPRRYVQPPE | GAGE-3 [4- |
| | | | 81] | | | | 23] |
| 2 | 2917 | FLWGPRAYAETSKMKVLEFL | MAGE- | 1 | 4240 | WRGRSTYYWPRPRRYVQPPE | GAGE-6 [3- |
| | | | B2 [273-292] | | | | 22]; GAGE- |
| | | | | | | | 7 [3-22] |
| 2 | 2918 | PREDAHFIYGYPKKGHGHSY | MART1 [2-21] | 1 | 4241 | ALSRHMSSLSHISPFSHSSH | GATA-3 [396- |
| | | | | | | | 415] |
| 2 | 2919 | KFRPGSVVVQLTLAFREGTI | MUC-1 [1093- | 1 | 4242 | DGGNKHFLRNQPLTFALQLH | gp100 [226- |
| | | | 1112] | | | | 245] |
| 2 | 2920 | MVLPDVFIRCVVFCLTVVCW | NA17-A [1- | 1 | 4243 | KKVNFLDMSLDDIIIYKELE | HOM-TES- |
| | | | 20] | | | | 85 [21-40] |
| 2 | 2921 | QYIPHLFSYSGHSPLLPQQA | NR6A1 [224- | 1 | 4244 | IQNHDIMHAIISPLRSANTV | KU-CT- |
| | | | 243] | | | | 1 [435-454] |
| 2 | 2922 | GQSQGSVLAFTRTFIATPGS | NXF2 [509- | 1 | 4245 | PDSRLLQLHITMPFSSPMEA | Lage-1 [83- |
| | | | 528] | | | | 102] |
| 2 | 2923 | LLKEFTVSGNILTIRLTAAD | NY-ESO- | 1 | 4246 | QRNVAIMKSIIPAIVHYSPD | LDHC [111- |
| | | | 1 [122-141] | | | | 130] |
| 2 | 2924 | EKDVTYSYGLGSCVKIESPC | ODF1 [183- | 1 | 4247 | VKVLEYVIKVSARVRFFFPS | MAGE- |
| | | | 202] | | | | A1 [277-296] |
| 2 | 2925 | PPTTGFMKHTPTKLRAPAYS | ODF3 [33-52] | 1 | 4248 | DPTSHSYVLVTSLNLSYDGI | MAGE- |
| | | | | | | | A11 [283-302] |
| 2 | 2926 | SNRIHTSAHVMSMGLLHFYK | ODF4 [121- | 1 | 4249 | GREDSVFAHPRKLLMQDLVQ | MAGE- |
| | | | 140] | | | | A2 [233-252] |
| 2 | 2927 | SLGAVVFSRVTNNVVLEAPF | OIP5 [100- | 1 | 4250 | FPVIFSKASSSLQLVFGIEL | MAGE- |
| | | | 119] | | | | A3 [147-166] |
| 2 | 2928 | DGPDTKRVCLRNEEQMKLPA | PAGE1 [77- | 1 | 4251 | VNARVRIAYPSLREAALLEE | MAGE- |
| | | | 96] | | | | A4 [294-313] |
| 2 | 2929 | GLTLWEMMTLSIPHINLSND | PBK [234- | 1 | 4252 | KKLLTQYFVQENYLEYRQVP | MAGE- |
| | | | 253] | | | | A6 [243-262] |
| 2 | 2930 | NRKYAFKAAHPNMRTYYFCT | PEPP2 [231- | 1 | 4253 | RAPEEAIWEALSVMGLYDGR | MAGE- |
| | | | 250] | | | | A8 [218-237] |
| 2 | 2931 | GKQQTVMAIATKIALQMNCK | PIWIL1 [592- | 1 | 4254 | PAQLEFMFQEALKLKVAELV | MAGE-A9 [97- |
| | | | 611] | | | | 116] |
| 2 | 2932 | CLRYYNILFRRTFKLLDFEQ | PIWIL3 [221- | 1 | 4255 | SSDPPRFQFLWGPRAYAETS | MAGE- |
| | | | 240] | | | | B2 [265-284] |
| 2 | 2933 | YFERKLLFSADVSYKVLRNE | PIWIL4 [252- | 1 | 4256 | LIMKTNMLVQFLMEMYKMKK | MAGE- |
| | | | 271] | | | | B3 [111-130] |
| 2 | 2934 | FKFIGLMILLTSAFSAGSGQ | PLAC1 [4-23] | 1 | 4257 | RPVSSFFSYTLASLLQSSHE | MAGE- |
| | | | | | | | C1 [777-796] |
| 2 | 2935 | RHSQTLKAMVQAWPFTCLPL | PRAME [64- | 1 | 4258 | ATVMASESLSVMSSNVSFSE | MAGE- |
| | | | 83] | | | | C2 [354-373] |
| 2 | 2936 | PGSPRSWLLLCPLSHVLFRA | PRSS55 [330- | 1 | 4259 | EIYNEYIYDLFVPVSSKFQK | MPHOSPH1 [277- |
| | | | 349] | | | | 296] |
| 2 | 2937 | MAGLALQPGTALLCYSCKAQ | PSCA [1-20] | 1 | 4260 | GVSFFFLSFHISNLQFNSSL | MUC-1 [1039- |
| | | | | | | | 1058] |
| 2 | 2938 | QVRGGMVFELANSIVLPFDC | PSMA [578- | 1 | 4261 | EEAFSRASLVSVYNSYPYYP | NKX3.1 [202- |
| | | | 597] | | | | 221] |
| 2 | 2939 | AASDNVFSTVPPAFINMAAT | SAGE1 [592- | 1 | 4262 | SLLRKKMLPEASLLIAIKPV | NLRP4 [263- |
| | | | 611] | | | | 282] |
| 2 | 2940 | EDANSCHSFQSAYLIVDRDE | SCRN1 [145- | 1 | 4263 | IERLIYLYHKFHQLKVSNEE | NR6A1 [369- |
| | | | 164] | | | | 388] |
| 2 | 2941 | LWTWWINFLFAAVVAWCTLI | SLCO6A1 [311- | 1 | 4264 | ADKDLYVAEALSTLESWRSR | ODF2 [428- |
| | | | 330] | | | | 447] |
| 2 | 2942 | QFIPSTMAAAAASGLSPLQL | SOX-6 [306- | 1 | 4265 | NSPLPFQWRITHSFRWMAQV | ODF4 [63-82] |
| | | | 325] | | | | |
| 2 | 2943 | KGIVLVALADGTLAIFHRGV | SPAG9 [1010- | 1 | 4266 | MGFLRRLIYRRRPMIYVESS | PAGE1 [1-20] |
| | | | 1029] | | | | |
| 2 | 2944 | MIITTWIVYILARKGVGLPF | SPATA19 [1- | 1 | 4267 | GPDMEAFQQELALLKIEDEP | PAGE2 [65- |
| | | | 20] | | | | 84] |
| 2 | 2945 | DSILRRPYVTCQPFWRKEME | SPO11 [340- | 1 | 4268 | RGDGQEAPDVVAFVAPGESQ | PAGE4 [12- |
| | | | 359] | | | | 31] |
| 2 | 2946 | FLKDHRISTFKNWPFLEGCA | Survivin [13- | 1 | 4269 | GTDVEAFQQELALLKIEDAP | PAGE5 [84- |
| | | | 32] | | | | 103] |
| 2 | 2947 | VTGYKMLYQNDLHLTPTLHL | TAG-1 [944- | 1 | 4270 | KNTPGIYTSLVNYNLWIEKV | PRSS55 [279- |
| | | | 963] | | | | 298] |
| 2 | 2948 | ECNVVIIAHCNLEPLVSRDP | TAG-2a [54- | 1 | 4271 | SNPIVLRMMNDQLMFLERAF | PSMA [656- |
| | | | 73] | | | | 675] |
| 2 | 2949 | IPVWGLQLLLPLLLPSFIHF | TEX101 [229- | 1 | 4272 | GSTGFSSRLAATQDLPFIHQ | RCAS1 [123- |
| | | | 248] | | | | 142] |
| 2 | 2950 | EPTFSFFSGPYMVMTNLVWN | TEX14 [247- | 1 | 4273 | KDSITVFVSNLPYSMQEPDT | SART3 [701- |
| | | | 266] | | | | 720] |
| 2 | 2951 | GLPIILHLFALSTLYFYKFF | TMEM31 [120- | 1 | 4274 | KLNQEYSQQFLTLFQQWDLD | SCP3a [120- |
| | | | 139] | | | | 139] |
| 2 | 2952 | KIKKIVHSIVSSFAFGLFGV | TPTE [80-99] | 1 | 4275 | TSELKTEGVSPYLMLIRLRK | se57-1 [316- |
| | | | | | | | 335] |
| 2 | 2953 | IKMRCEFLACWPAFTVLGEA | TRAG-3 [30- | 1 | 4276 | KERQLSTMITQLISLREQLL | SOX-6 [187- |
| | | | 49] | | | | 206] |
| 2 | 2954 | QNYSSNAYHMSSTMKPNTKC | TSGA10 [651- | 1 | 4277 | PDNIPAFAAAYFESLLEKRE | SP17 [32-51] |
| | | | 670] | | | | |
| 2 | 2955 | IWRDIDFAHEAPAFLPWHRL | TYR [194- | 1 | 4278 | KKMKTSESSTILVVRYRRNV | SPAN-Xc [40- |
| | | | 213] | | | | 59] |
| 2 | 2956 | ASSGQARMFPNAPYLPSCLE | WT1 [120- | 1 | 4279 | TRIQFIRWSHTRIFQVPSEM | 5PATA19 [112- |
| | | | 139] | | | | 131] |
| 2 | 2957 | KKIRIQLRSQCATWKVICKS | XAGE- | 1 | 4280 | AEIQALTFLSSDYLSRVYLP | SPO11 [368- |
| | | | 1c [102-121] | | | | 387] |
| 3 | 2958 | LRRLELASNHFLYLPRDVLA | 5T4 [212- | 1 | 4281 | VERPQMTFGRLQGISPKIMP | SSX-2 [94- |
| | | | 231] | | | | 113] |
| 3 | 2959 | CDTDYIQYPNYCSFKSQQCL | ACRBP [477- | 1 | 4282 | QRPQMTFGRLQGIFPKIMPK | SSX-3 [95- |
| | | | 496] | | | | 114] |
| 3 | 2960 | WNEPQMVFPNIVNYLPCKEN | ACTL8 [22- | 1 | 4283 | EFEETAKKVRRAIEQLAAMD | Survivin [123- |
| | | | 41] | | | | 142] |
| 3 | 2961 | RYIENIYHSKPMRWPFFLFI | ADAM2 [674- | 1 | 4284 | LFLRSQEAAATVQLFQEEHR | SYCE1 [229- |
| | | | 693] | | | | 248] |
| 3 | 2962 | PRCIIILVLQEPTPFRISVT | BAGE-3 [59- | 1 | 4285 | ESGQYRANEGTSSIVMEIQK | TAF7L [377- |
| | | | 78] | | | | 396] |
| 3 | 2963 | ECIEDFNTMFSNCYLYNKPG | BRDT [93- | 1 | 4286 | SSEVLEYMNQLSASLKETYA | TDRD1 [285- |
| | | | 112] | | | | 304] |
| 3 | 2964 | EACRETYMEFLWQYKSSADK | CDCA1 [132- | 1 | 4287 | GKINQNYASIITEAFPKPKD | TEX15 [368- |
| | | | 151] | | | | 387] |
| 3 | 2965 | TRRGKSTQPCEYYFRVYHSL | COX6B2 [47- | 1 | 4288 | VSRAAQMAVPSSRILQLSKP | THEG [255- |
| | | | 66] | | | | 274] |
| 3 | 2966 | NQDSLKYYYVCQYCPAGNNM | CRISP2 [157- | 1 | 4289 | DLDLTYVTERIIAMSFPSSG | TPTE [236- |
| | | | 176] | | | | 255] |
| 3 | 2967 | ECYQFKFKYTNNGPLMDFIS | CT46 [113- | 1 | 4290 | EELQKVQFEKVSALADLSST | TSGA10 [492- |
| | | | 132] | | | | 511] |
| 3 | 2968 | VVARRYPASGIHFVLLDMVH | CT47 [109- | 1 | 4291 | HNRESYMVPFIPLYRNGDFF | TYR [420-439] |
| | | | 128] | | | | |
| 3 | 2969 | WELVIRAAVAGFFAVLFLWR | CTAGE2 [12- | 1 | 4292 | QTPGINLDLGSGVKVKIIPK | XAGE- |
| | | | 31] | | | | 1c [125- |
| | | | | | | | 144]; XAGE- |
| | | | | | | | 1 [46-65]; |
| | | | | | | | XAGE-1b [46- |
| | | | | | | | 65] |
| 3 | 2970 | CNDCNMAFVTSGELVRHRRY | CTCFL [315- | 1 | 4293 | SRQKKIRIQLRSQVLGREMR | XAGE-1d [20- |
| | | | 334] | | | | 39] |
| 3 | 2971 | SSILCALIVFWKYRRFQRNT | CXorf61 [9- | 1 | 4294 | MSWRGRSTYRPRPRRSLQPP | XAGE-2 [1- |
| | | | 28] | | | | 20] |
| 3 | 2972 | ADKPVLAIQVLGTVKWFNVR | DBPC [85- | 1 | 4295 | MIWRGRSTYRPRPRRSVPPP | XAGE-3 [1- |
| | | | 104] | | | | 20] |
| 3 | 2973 | VLLLLLSTLVIPSAAAPIHD | DKKL1 [16- | 2 | 4296 | SHKTPFVSPLLASQSLSIGN | ACRBP [399- |
| | | | 35] | | | | 418] |
| 3 | 2974 | CKKCNLIAERQRVMAAQVAL | DMRT1 [102- | 2 | 4297 | RIVEIVVVIDNYLYIRYERN | ADAM29 [198- |
| | | | 121] | | | | 217] |
| 3 | 2975 | TNTVEVITSAPGAMLASWAR | DPPA2 [172- | 2 | 4298 | STPPSAYGSVKAYTNFDAER | ANXA2 [18- |
| | | | 191] | | | | 37] |
| 3 | 2976 | MNDMPIYMYSVCNVMSGDQD | EPHA2 [59- | 2 | 4299 | QLLQARLMKEESPVVSWRLE | BAGE-1 [13- |
| | | | 78] | | | | 32]; BAGE- |
| | | | | | | | 2 [13-32]; |
| | | | | | | | BAGE-3 [13- |
| | | | | | | | 32]; BAGE- |
| | | | | | | | 5 [13-32] |
| 3 | 2977 | IWILLFVCYYLSYYLCSGSS | FMR1NB [76- | 2 | 4300 | AKYSSVYMEAEATALLSDTS | CABYR [372- |
| | | | 95] | | | | 391] |
| 3 | 2978 | YYWPRPRRYVQPPEMIGPMR | GAGE-7 [9- | 2 | 4301 | ENHPKSMTMMPALFKENRND | CAGE1 [756- |
| | | | 28] | | | | 775] |
| 3 | 2979 | HHPGLSHSYMDAAQYPLPEE | GATA-3 [27- | 2 | 4302 | ATAQLQRTPMSALVFPNKIS | CT46 [2-21] |
| | | | 46] | | | | |
| 3 | 2980 | GECCKYSYKGLRSVCVYGGG | HAGE [334- | 2 | 4303 | ALKKLIHAAKLNASLKTLEG | cTAGE5 [312- |
| | | | 353] | | | | 331]; |
| | | | | | | | CTAGE2 [282- |
| | | | | | | | 301] |
| 3 | 2981 | ISKVMEMYQPSAVVLQCGAD | HDAC2 [246- | 2 | 4304 | NTGEMSSNSTALALVRPSSS | CXorf61 [27- |
| | | | 265] | | | | 46] |
| 3 | 2982 | KPHRLALTHSLVLHYGLYKK | HDAC3 [25- | 2 | 4305 | GQGSLLFYDIRAQRFLEERL | DCAF12 [358- |
| | | | 44] | | | | 377] |
| 3 | 2983 | AVLPCTFTTSAALINLNVIW | IGFS11 [40- | 2 | 4306 | TELHPRVAFWIIKLPRRRSH | DKKL1 [161- |
| | | | 59] | | | | 180] |
| 3 | 2984 | QDMDCVYYNWQYLLCSWKPG | IL13RA2 [141- | 2 | 4307 | SDSTYYSSFYQPSLFPYYNN | DMRT1 [205- |
| | | | 160] | | | | 224] |
| 3 | 2985 | QCAGEFVITFPRAYHSGFNQ | JARID1B [573- | 2 | 4308 | GKKIEVYLRLHRHAYPEQRQ | DPPA2 [113- |
| | | | 592] | | | | 132] |
| 3 | 2986 | GVPVAVHKHALHDGLRQVAG | KAAG1 [11- | 2 | 4309 | EKEKDVRSLSKKRKKSYPDD | FAM133A [168- |
| | | | 30] | | | | 187] |
| 3 | 2987 | PVSGPFLVKTGYAFVDCPDE | KOC1 [28-47] | 2 | 4310 | RRQFEFSVDSFQIVLDPMLD | FAM46D [166- |
| | | | | | | | 185] |
| 3 | 2988 | PDFSMYVYEVTKSILPITNI | KU-CT- | 2 | 4311 | EVMRRQLYAVNRRLRALEEQ | FATE1 [135- |
| | | | 1 [721-740] | | | | 154] |
| 3 | 2989 | LHITMPFSSPMEAELVRRIL | Lage-1 [90- | 2 | 4312 | NLKVNFFFERIMKYERLARI | FBXO39 [282- |
| | | | 109] | | | | 301] |
| 3 | 2990 | PRIETILMMYTRNNLNCAEP | LIPI [60-79] | 2 | 4313 | KLQELSAASPTISSFSPRLE | FSIP1 [326- |
| | | | | | | | 345] |
| 3 | 2991 | DPACYEFLWGPRALVETSYV | MAGE- | 2 | 4314 | GWESGLVAMESAFHLEKNVN | FTHL17 [93- |
| | | | A3 [265-284] | | | | 112] |
| 3 | 2992 | PDPESVFRAALSKKVADLIH | MAGE-A5 [99- | 2 | 4315 | EEMRSHYVAQTGILWLLMNN | GAGE-1 [109- |
| | | | 118] | | | | 128] |
| 3 | 2993 | AALSRKVAKLVHFLLLKYRA | MAGE- | 2 | 4316 | MNLSRGKSTYYWPRPRRYVQ | GAGE-3 [1- |
| | | | A6 [107-126] | | | | 20] |
| 3 | 2994 | DPAGHSYILVTALGLSCDSM | MAGE- | 2 | 4317 | MSWRGRSTYYWPRPRRYVQP | GAGE-6 [1- |
| | | | A9 [169-188] | | | | 20]; GAGE- |
| | | | | | | | 7 [1-20] |
| 3 | 2995 | ALPKSGLLMSLLVVIFMNGN | MAGE- | 2 | 4318 | EKFKKAMTIGDVSLVQELLD | GASZ [48-67] |
| | | | B6 [281-300] | | | | |
| 3 | 2996 | KVVEFLAMLKNTVPITFPSS | MAGE- | 2 | 4319 | KPHRIRMTHNLLLNYGLYRK | HDAC2 [32- |
| | | | C1 [1083- | | | | 51]; |
| | | | 1102] | | | | HDAC1 [31- |
| | | | | | | | 50] |
| 3 | 2997 | AGREHFVYGEPRELLTKVWV | MAGE- | 2 | 4320 | MASFRKLTLSEKVPPNHPSR | HOM-TES- |
| | | | C2 [260-279] | | | | 85 [1-20] |
| 3 | 2998 | IGCWYCRRRNGYRALMDKSL | MART1 [43- | 2 | 4321 | PIRSSYFTFQLQNIVKPLPP | IL13RA2 [222- |
| | | | 62] | | | | 241] |
| 3 | 2999 | AYTSVLYFNPWMRIFIQAKR | MORC1 [239- | 2 | 4322 | YENDIASMNLQAHLIPGLNL | KOC1 [351- |
| | | | 258] | | | | 370] |
| 3 | 3000 | LVALAIVYLIALAVCQCRRK | MUC-1 [1170- | 2 | 4323 | ATVLTNMAMQEPLRLNIQNH | KU-CT- |
| | | | 1189] | | | | 1 [419-438] |
| 3 | 3001 | GSYRKWMCFSESQVQPTQKQ | NA17-A [32- | 2 | 4324 | GAVLKDFTVSGNLLFMSVRD | Lage-1 [120- |
| | | | 51] | | | | 139] |
| 3 | 3002 | SLVSVYNSYPYYPYLYCVGS | NKX3.1 [209- | 2 | 4325 | AQFYNDFVNISSIGLTYFQS | LIPI [409- |
| | | | 228] | | | | 428] |
| 3 | 3003 | ESSYVFLHVCIQEFCAALFY | NLRP4 [445- | 2 | 4326 | VSARVRFFFPSLREAALREE | MAGE- |
| | | | 464] | | | | A1 [286-305] |
| 3 | 3004 | AFREQYMGMSVPPHYQYIPH | NR6A1 [209- | 2 | 4327 | ESVIRNYEDHFPLLFSEASE | MAGE-A10 [162-181] |
| | | | 228] | | | | |
| 3 | 3005 | HTPHKTLMPYASLFQSHSCK | NYC- | 2 | 4328 | PDLESEFQAAISRKMVELVH | MAGE-A2 [99- |
| | | | TSPG [515- | | | | 118] |
| | | | 534] | | | | |
| 3 | 3006 | LQQLSLLMWITQCFLPVFLA | NY-ESO- | 2 | 4329 | VVGNWQYFFPVIFSKASSSL | MAGE- |
| | | | 1 [153-172] | | | | A3 [139-158] |
| 3 | 3007 | CDLHPYPYCLCYSKRSRSCG | ODF1 [47-66] | 2 | 4330 | VVGNWQYFFPVIFSKASDSL | MAGE- |
| | | | | | | | A6 [139-158] |
| 3 | 3008 | RGPIMALYSSPGPKYLIPPT | ODF3 [16-35] | 2 | 4331 | DGREHSVYWKLRKLLTQEWV | MAGE- |
| | | | | | | | A8 [235-254] |
| 3 | 3009 | QCHAVLADSVHLAWDLSRSL | OIP5 [82- | 2 | 4332 | AHAETSYEKVINYLVMLNAR | MAGE- |
| | | | 101] | | | | A9 [276-295] |
| 3 | 3010 | QEDRLYLVGNVCILRTQLLQ | PASD1 [163- | 2 | 4333 | TEEEIWKFMNVLGAYDGEEH | MAGE- |
| | | | 182] | | | | B1 [216-235] |
| 3 | 3011 | GFTTSILQYENSIMLCTDVS | PIWIL1 [251- | 2 | 4334 | TNKKKVSFSSPLILGATIQK | MAGE-B3 [33- |
| | | | 270] | | | | 52] |
| 3 | 3012 | CIPFYNVVFRRVMKLLDMKL | PIWIL2 [319- | 2 | 4335 | VAARRGTTAMTSAYSRATSS | MAGE- |
| | | | 338] | | | | B4 [321-340] |
| 3 | 3013 | NHVQPHAYQFTYRVTECGIR | PLAC1 [64- | 2 | 4336 | PQILNRTSQHLVVAFGVELK | MAGE- |
| | | | 83] | | | | B6 [234-253] |
| 3 | 3014 | LQHLIGLSNLTHVLYPVPLE | PRAME [427- | 2 | 4337 | NPASSFFSSALLSIFQSSPE | MAGE- |
| | | | 446] | | | | C1 [130-149] |
| 3 | 3015 | CWTARIRAVGLLTVISKGCS | PSCA [39-58] | 2 | 4338 | SSASSTLYLVFSPSSFSTSS | MAGE-C2 [40- |
| | | | | | | | 59] |
| 3 | 3016 | SAGCVFYEIASLQPLFPGVN | RAGE-1 [187- | 2 | 4339 | RSQAGMFIYSNNRLIKMHEK | MORC1 [363- |
| | | | 206] | | | | 382] |
| 3 | 3017 | MAITQFRLFKFCTCLATVFS | RCAS1 [1-20] | 2 | 4340 | YEQANLNMANSIKFSVWVSF | MPHOSPH1 [256- |
| | | | | | | | 275] |
| 3 | 3018 | PNNVLSTVQPVIIYLTATGI | SAGE1 [312- | 2 | 4341 | PQKRSRAAFSHTQVIELERK | NKX3.1 [123- |
| | | | 331] | | | | 142] |
| 3 | 3019 | CKALMRFIMVTSVISLILLV | SLCO6A1 [451- | 2 | 4342 | GINNVSFSGQSVLLFEVLFY | NLRP4 [673- |
| | | | 470] | | | | 692] |
| 3 | 3020 | RALELHPFSMKPLLRRAMAY | SPAG1 [512- | 2 | 4343 | KRASQYSGQLKVLIAENTML | NY-BR- |
| | | | 531] | | | | 1 [1103- |
| | | | | | | | 1122] |
| 3 | 3021 | SAQKFSLILKILSMIYKLVQ | SPO11 [107- | 2 | 4344 | WTLSRFFSYLRSWDVDDLLL | NYC- |
| | | | 126] | | | | TSPG [322- |
| | | | | | | | 341] |
| 3 | 3022 | TSEKINMISGVLQRYCRFGS | SSX-3 [147- | 2 | 4345 | GDGPYSTFLTSSPIRSRSPP | ODF2 [809- |
| | | | 166] | | | | 828] |
| 3 | 3023 | MGAPTLPPAWQPFLKDHRIS | Survivin [1- | 2 | 4346 | NDQESSQPVGSVIVQEPTEE | PAGE2 [16- |
| | | | 20] | | | | 35] |
| 3 | 3024 | DKKTFYKTADISQMLVCTAD | TAF7L [167- | 2 | 4347 | MSARVRSRSRGRGDGQEAPD | PAGE4 [1-20] |
| | | | 186] | | | | |
| 3 | 3025 | LSNRLLLRLECNVVIIAHCN | TAG-2a [45- | 2 | 4348 | RGNDQESSQPVGPVIVQQPT | PAGE5 [33- |
| | | | 64] | | | | 52] |
| 3 | 3026 | ECYQPYYLPGYRYLNSWRPS | TEKT5 [34- | 2 | 4349 | NHPVRFLQAQPIVPVQRAAE | PASD1 [599- |
| | | | 53] | | | | 618] |
| 3 | 3027 | YWTERFLEDTTLTITVPAVS | THEG [172- | 2 | 4350 | LVGRNFYDPTSAMVLQQHRL | PIWIL2 [338- |
| | | | 191] | | | | 357] |
| 3 | 3028 | PYRLPALFELYPEFLLVFKE | TMEM31 [84- | 2 | 4351 | TKFKQSRAMNFDFPFKKGSG | RAGE-1 [227- |
| | | | 103] | | | | 246] |
| 3 | 3029 | DVKVQFFYSNLPTYYDNCSF | TPTE [475- | 2 | 4352 | QLEPDYFKDMTPTIRKTQKI | RCAS1 [89- |
| | | | 494] | | | | 108] |
| 3 | 3030 | ICAGTIIASQWVLTVAHCLI | TSP50 [137- | 2 | 4353 | KELEELRAAFTRALEYLKQE | SART3 [440- |
| | | | 156] | | | | 459] |
| 3 | 3031 | LFVWMHYYVSMDALLGGSEI | TYR [175- | 2 | 4354 | NNNIEKMITAFEELRVQAEN | SCP-1 [215- |
| | | | 194] | | | | 234] |
| 3 | 3032 | GCNKRYFKLSHLQMHSRKHT | WT1 [329- | 2 | 4355 | METQQQEIASVRKSLQSMLF | SCP3a [217- |
| | | | 348] | | | | 236] |
| 3 | 3033 | VICKSCISQTPGINLDLGSG | XAGE- | 2 | 4356 | EISILQEQISHLQFVIHSQH | se57-1 [235- |
| | | | 1c [117-136] | | | | 254] |
| 3 | 3034 | QILELLVLEQFLTILPGDLQ | ZNF165 [87- | 2 | 4357 | KEEVAAVKIQAAFRGHIARE | SP17 [113- |
| | | | 106] | | | | 132] |
| 4 | 3035 | HYRCSNHVYYAKRVLCSQPV | ACRBP [108- | 2 | 4358 | EKDPSLVYQHLLYLSKAERF | SPAG1 [863- |
| | | | 127] | | | | 882] |
| 4 | 3036 | HVPSVLLADQLQMSLYASGL | ACTL8 [123- | 2 | 4359 | NPLQMEEEEFMEIMVEIPAK | SPAN-Xc [78- |
| | | | 142] | | | | 97] |
| 4 | 3037 | CAIVTFMNKTLGTFSIAVAH | ADAM29 [313- | 2 | 4360 | EKISYVYMKRNYKAMTKLGF | SSX-1 [44- |
| | | | 332] | | | | 63] |
| 4 | 3038 | LNEELDFLVTSYEEIIECAD | CAGE1 [697- | 2 | 4361 | EWEKMKASEKIFYVYMKRKY | SSX-2 [36- |
| | | | 716] | | | | 55] |
| 4 | 3039 | MARALVQLWAICMLRVALAT | CALR3 [1-20] | 2 | 4362 | EKIVYVYMKRKYEAMTKLGF | SSX-3 [44- |
| | | | | | | | 63] |
| 4 | 3040 | EVMYPHLMEGFLPFSNLVTH | CDCA1 [72- | 2 | 4363 | VERPQMTFGSLQRIFPKIMP | SSX-4 [94- |
| | | | 91] | | | | 113] |
| 4 | 3041 | RVYHSLCPISWVESWNEQIK | COX6B2 [61- | 2 | 4364 | EKIIYVYMKRKYEAMTKLGF | SSX-5 [44- |
| | | | 80] | | | | 63] |
| 4 | 3042 | SSDPTSWSSAIQSWYDEILD | CRISP2 [101- | 2 | 4365 | EPRVEVLINRINEVQQAKKK | SYCE1 [50- |
| | | | 120] | | | | 69] |
| 4 | 3043 | DSPSYQKRQRMALLARKQGA | CT45 [23-42] | 2 | 4366 | NLTLKNHFQSVLEQLELQEK | TAF7L [424- |
| | | | | | | | 443] |
| 4 | 3044 | VKRLLAVSVSCITYLRGIFP | CT46 [30-49] | 2 | 4367 | WAERIMFSDLRSLQLKKTME | TDRD1 [246- |
| | | | | | | | 265] |
| 4 | 3045 | VISFVLFLFGGNNFIQNFYL | CTAGE1 [12- | 2 | 4368 | TLQEIFQAENTIMLLERSIM | TEKT5 [363- |
| | | | 31] | | | | 382] |
| 4 | 3046 | SHPYGFPWELVIRAAVAGFF | CTAGE2 [5- | 2 | 4369 | TLVELQMMMETIQFIENKKR | TEX15 [1689- |
| | | | 24] | | | | 1708] |
| 4 | 3047 | RPDSNLYGFPWELVICAAVV | CTAGE5 [31- | 2 | 4370 | VTKKVVASPRIISLAKPKVR | THEG [326- |
| | | | 50] | | | | 345] |
| 4 | 3048 | SPPAPARRHLLVLLLLLSTL | DKKL1 [5-24] | 2 | 4371 | VIMHSRYRAQRFWSWVGQAN | TSP50 [186- |
| | | | | | | | 205] |
| 4 | 3049 | SQDSGLVSLSSSSPISNKST | DMRT1 [329- | 2 | 4372 | DEDEDMLSYMVSLEVGEEKH | TSPY1 [175- |
| | | | 348] | | | | 194] |
| 4 | 3050 | TSAPGAMLASWARIAARAVQ | DPPA2 [179- | 2 | 4373 | VAKKGKAVRRGRRGKKGAAT | VCX [38-57] |
| | | | 198] | | | | |
| 4 | 3051 | ECVCENYKLAVNCFVNNNRQ | EpCAM [26- | 2 | 4374 | KKKNQQLKVGILHLGSRQKK | XAGE-1 [5- |
| | | | 45] | | | | 24]; XAGE- |
| | | | | | | | 1b [5-24]; |
| | | | | | | | XAGE-1d [5- |
| | | | | | | | 24]; XAGE- |
| | | | | | | | 1c [84-103] |
| 4 | 3052 | ESTVCLMSYERRQILHLITM | FAM46D [308- | 2 | 4375 | PRRSLQPPELIGAMLEPTDE | XAGE-2 [13- |
| | | | 327] | | | | 32] |
| 4 | 3053 | SHYVAQTGILWLLMNNCFLN | GAGE-1 [113- | 2 | 4376 | KSFKSPKLAKHAAVFSGDKT | ZNF165 [324- |
| | | | 132] | | | | 343] |
| 4 | 3054 | LLKERDITLRHLLTMREDEF | GASZ [294- | 3 | 4377 | IKVKKLLFSKHLPVFTYTDQ | ADAM29 [67- |
| | | | 313] | | | | 86] |
| 4 | 3055 | SHISPFSHSSHMLTTPTPMH | GATA-3 [405- | 3 | 4378 | SGEKNIFLASFVHEYSRRHP | AFP [344-363] |
| | | | 424] | | | | |
| 4 | 3056 | ATWPHSVHRLAQSYLKEPMI | HAGE [428- | 3 | 4379 | SEGIMTYANSVVSDMMVSIM | AKAP-3 [282- |
| | | | 447] | | | | 301] |
| 4 | 3057 | NVARCWTYETAVALDCEIPN | HDAC2 [308- | 3 | 4380 | DQVNIDYLMNRPQNLRLEMT | AKAP-4 [161- |
| | | | 327] | | | | 180] |
| 4 | 3058 | NVARCWTYETSLLVEEAISE | HDAC3 [302- | 3 | 4381 | RDKVLIRIMVSRSEVDMLKI | ANXA2 [284- |
| | | | 321] | | | | 303] |
| 4 | 3059 | RQQSKAHRHRHRRGYSRCRS | HOM-TES- | 3 | 4382 | QARLMKEESPVVSWRLEPED | BAGE-1 [16- |
| | | | 85 [62-81] | | | | 35]; BAGE- |
| | | | | | | | 5 [16-35]; |
| | | | | | | | BAGE-2 [16- |
| | | | | | | | 35]; BAGE- |
| | | | | | | | 3 [16-35] |
| 4 | 3060 | APLLLLSLHGVAASLEVSES | IGFS11 [9- | 3 | 4383 | QADIEVMSTVHISSVYNDVP | CABYR [282- |
| | | | 28] | | | | 301] |
| 4 | 3061 | LAIGCLYTFLISTTFGCTSS | IL13RA2 [6- | 3 | 4384 | SRLEKLLTQVRNLQFMSENE | CAGE1 [507- |
| | | | 25] | | | | 526] |
| 4 | 3062 | AVHKHALHDGLRQVAGPGAA | KAAG1 [15- | 3 | 4385 | QTTQNGRFYAISARFKPFSN | CALR3 [67- |
| | | | 34] | | | | 86] |
| 4 | 3063 | HVKKGIFYHRALLFKALADR | KU-CT- | 3 | 4386 | TVFGEKSVITLSSIFTKDLV | CCDC62 [385- |
| | | | 1 [789-808] | | | | 404] |
| 4 | 3064 | WREEGFPVGLKLAVLGIFII | LEMD1 [147- | 3 | 4387 | GQKYEKIFEMLEGVQGPTAV | CT45 [138- |
| | | | 166] | | | | 157] |
| 4 | 3065 | PEEVIWEALNMMGLYDGMEH | MAGE- | 3 | 4388 | GNNFIQNFYLPQNYIDQFLL | CTAGE1 [22- |
| | | | A10 [242- | | | | 41] |
| | | | 261] | | | | |
| 4 | 3066 | KIIDLVHLLLRKYRVKGLIT | MAGE- | 3 | 4389 | TERLLKMKDWAAMLGEDITD | CTAGE5 [268- |
| | | | A11 [225- | | | | 287] |
| | | | 244] | | | | |
| 4 | 3067 | PACYEFLWGPRALVETSYVK | MAGE- | 3 | 4390 | SVKRGTMVRAARALLSAVTR | CTNNA2 [117- |
| | | | A12 [266- | | | | 136] |
| | | | 285] | | | | |
| 4 | 3068 | DPIGHLYIFATCLGLSYDGL | MAGE- | 3 | 4391 | RNGVIDYTIFFTLDSVKLPD | CXorf48 [188- |
| | | | A3 [170-189] | | | | 207] |
| 4 | 3069 | PACYEFLWGPRALIETSYVK | MAGE- | 3 | 4392 | VKRSLVYYLKNREVRLQNET | DCAF12 [42- |
| | | | A6 [266- | | | | 61] |
| | | | 285]; MAGE- | | | | |
| | | | A2 [266-285] | | | | |
| 4 | 3070 | RVQAAAMLNDGSSAMGRKCS | MAGE- | 3 | 4393 | PGQTLIYYVDEKAPEFSMQG | EpCAM [244- |
| | | | B3 [317-336] | | | | 263] |
| 4 | 3071 | KCVRREYKPYFPQILNRTSQ | MAGE- | 3 | 4394 | NRVAYMNPIAMARWRGPTQS | FAM133A [6- |
| | | | B6 [223-242] | | | | 25] |
| 4 | 3072 | HSYTTAERAAGIGILTVILG | MART1 [19- | 3 | 4395 | KNLELKFVSSLRRQFEFSVD | FAM46D [155- |
| | | | 38] | | | | 174] |
| 4 | 3073 | GKGKICMIVNISQCYLAYDE | MPHOSPH1 [446- | 3 | 4396 | GENQEHLVIAEMMELGSRSR | FATE1 [26- |
| | | | 465] | | | | 45] |
| 4 | 3074 | WSDNKIFRDRFLYTFYFCCR | NLRP4 [172- | 3 | 4397 | SPQFKKTMSTFHNLVSLNLN | FBXO39 [213- |
| | | | 191] | | | | 232] |
| 4 | 3075 | RFTPVDFHYVRNRACFFVQD | NXF2 [151- | 3 | 4398 | RPGSRSSNASLEVLSTEPGS | FSIP1 [21- |
| | | | 170] | | | | 40] |
| 4 | 3076 | ILSVVAKLLSHGAVIEVHNK | NY-BR-1 [95- | 3 | 4399 | DDVALENFFRYFLRLSDDKM | FTHL17 [45- |
| | | | 114] | | | | 64] |
| 4 | 3077 | AEDWNLYWRTSSFRMTEHNS | NYC- | 3 | 4400 | EKDHIFSSYTAFGDLEVFLH | GASZ [265- |
| | | | TSPG [118- | | | | 284] |
| | | | 137] | | | | |
| 4 | 3078 | AADHRQLQLSISSCLQQLSL | NY-ESO- | 3 | 4401 | EQLLQSASMELKFLIIQNAA | Glypican- |
| | | | 1 [139-158] | | | | 3 [90-109] |
| 4 | 3079 | LGSKKYSYMNICKEFSLPPC | ODF1 [161- | 3 | 4402 | TLIGANASFSIALNFPGSQK | gp100 [76- |
| | | | 180] | | | | 95] |
| 4 | 3080 | ENGKVTFIFSTLMLFPINIW | ODF4 [148- | 3 | 4403 | ATQRDLIATQRDLIVTQRDL | HOM-TES- |
| | | | 167] | | | | 85 [261-280] |
| 4 | 3081 | GDGPDVREGIMPTFDLTKVL | PAGE2 [85- | 3 | 4404 | WAIGLSVMDLVGSILKNLRR | LDHC [250- |
| | | | 104] | | | | 269] |
| 4 | 3082 | NEFPVVFSGLFSSHLCADFA | PASD1 [139- | 3 | 4405 | SEELNIILQGNIILSTEKSK | LEMD1 [67- |
| | | | 158] | | | | 86] |
| 4 | 3083 | ILTIPMHFWALFYPKRAMDQ | PIWIL2 [598- | 3 | 4406 | RALAETSYVKVLEYVIKVSA | MAGE- |
| | | | 617] | | | | A1 [269-288] |
| 4 | 3084 | YVTSVLQYENSITLCADVSH | PIWIL3 [267- | 3 | 4407 | DGMEHLIYGEPRKLLTQDWV | MAGE-A10 [257-276] |
| | | | 286] | | | | |
| 4 | 3085 | PAPCQYAHKLTFLVAQSIHK | PIWIL4 [820- | 3 | 4408 | YAGREHFLFGEPKRLLTQNW | MAGE-A11 [344-363] |
| | | | 839] | | | | |
| 4 | 3086 | MVTVHPFMLNNDVCVHFHEL | PLAC1 [36- | 3 | 4409 | PDLETSFQVALSRKMAELVH | MAGE- |
| | | | 55] | | | | A12 [99-118] |
| 4 | 3087 | EKEEQYIAQFTSQFLSLQCL | PRAME [279- | 3 | 4410 | PQGASSFSTTINYTLWRQSD | MAGE-A2 [65- |
| | | | 298] | | | | 84] |
| 4 | 3088 | SILNKWWILTAAHCLYSEEL | PR5555 [96- | 3 | 4411 | ETSYVKVLEHVVRVNARVRI | MAGE- |
| | | | 115] | | | | A4 [281- |
| | | | | | | | 300]; MAGE- |
| | | | | | | | A8 [283-302] |
| 4 | 3089 | LQPAAAILALLPALGLLLWG | PSCA [91- | 3 | 4412 | EALDEKVAELVRFLLRKYQI | MAGE- |
| | | | 110] | | | | A8 [110-129] |
| 4 | 3090 | VDCTPLMYSLVHNLTKELKS | PSMA [464- | 3 | 4413 | NSDPPRYQFLWGPRAYAETT | MAGE- |
| | | | 483] | | | | B1 [262-281] |
| 4 | 3091 | ALICELMDMNIYELIRGRRY | RAGE-1 [79- | 3 | 4414 | FPEILKKASEGLSVVFGLEL | MAGE- |
| | | | 98] | | | | B2 [149-168] |
| 4 | 3092 | FKFCTCLATVFSFLKRLICR | RCAS1 [9-28] | 3 | 4415 | YDGKKHFIFGEPRKLITQDL | MAGE- |
| | | | | | | | B3 [233-252] |
| 4 | 3093 | GDPSCVIMQNWARIEARLCN | SART3 [468- | 3 | 4416 | SSSVLRDTASSSLAFGIPQE | MAGE-B4 [42- |
| | | | 487] | | | | 61] |
| 4 | 3094 | LLVFIIFVRCNPVQFAGINE | SLCO6A1 [468- | 3 | 4417 | ADIKKQAEIAHLYIASLPDP | MPHOSPH1 [687- |
| | | | 487] | | | | 706] |
| 4 | 3095 | NKECAIYTNRALCYLKLCQF | SPAG1 [654- | 3 | 4418 | ASPQETQSAFSIPVSTLSSS | NXF2 [544- |
| | | | 673] | | | | 563] |
| 4 | 3096 | IRWSHTRIFQVPSEMTEDIM | SPATA19 [117- | 3 | 4419 | HHIHEQIMEYIRKLSKNHQN | NY-BR- |
| | | | 136] | | | | 1 [195-214] |
| 4 | 3097 | SWENIKFEDSVGLQMVSHCT | SPO11 [77- | 3 | 4420 | STNRSMQNYVQFLKSSYANV | ODF2 [788- |
| | | | 96] | | | | 807] |
| 4 | 3098 | AMTKLGFKVTLPPFMCNKQA | SSX-1 [57- | 3 | 4421 | LGQDGRLLSSTLSLSSNRSL | ODF4 [39-58] |
| | | | 76] | | | | |
| 4 | 3099 | AMTKLGFKATLPPFMCNKRA | SSX-2 [57- | 3 | 4422 | QAVPAFQGPDMEAFQQELAL | PAGE2 [58- |
| | | | 76] | | | | 77] |
| 4 | 3100 | AMTKLGFKATLPPFMRNKRV | SSX-5 [57- | 3 | 4423 | EDPKDRPSAAHIVEALETDV | PBK [303-322] |
| | | | 76] | | | | |
| 4 | 3101 | KHSSGCAFLSVKKQFEELTL | Survivin [79- | 3 | 4424 | GWEEAYTFEGARYYINHNER | PEPP2 [61- |
| | | | 98] | | | | 80] |
| 4 | 3102 | KAEELLAAAAQRHQQLQQKC | SYCE1 [249- | 3 | 4425 | QKSIAGFVASTNAELTKWYS | PIWIL3 [663- |
| | | | 268] | | | | 682] |
| 4 | 3103 | PASASLFQDTCAGCASLLHG | TAG-2a [74- | 3 | 4426 | YIPDLASRRLRIALLYSHSE | PIWIL4 [130- |
| | | | 93] | | | | 149] |
| 4 | 3104 | WYRASVLAYASEESVLVGYV | TDRD1 [558- | 3 | 4427 | DFKRANMDNDIALLLLASPI | PRSS55 [147- |
| | | | 577] | | | | 166] |
| 4 | 3105 | LRPPTILPTLRSALFSRYSP | TEKT5 [72- | 3 | 4428 | QSGAAVVHEIVRSFGTLKKE | PSMA [389- |
| | | | 91] | | | | 408] |
| 4 | 3106 | VEVKGCTAMIGCRLMSGILA | TEX101 [180- | 3 | 4429 | KNPDFKVFRYSTSLEKHKLF | SART3 [785- |
| | | | 199] | | | | 804] |
| 4 | 3107 | IYSFGFGKAMPWFQFYLTGA | TEX14 [205- | 3 | 4430 | ASLEIELSNLKAELLSVKKQ | SCP-1 [747- |
| | | | 224] | | | | 766] |
| 4 | 3108 | CGKGYAWISPCKMSLHFCLC | THEG [148- | 3 | 4431 | AKRKRLEMYTKASLKTSNQK | SCP3a [87- |
| | | | 167] | | | | 106] |
| 4 | 3109 | WPTEWIFNPYRLPALFELYP | TMEM31 [76- | 3 | 4432 | DSEFFLTTASGVSVLPQNRS | SCRN1 [268- |
| | | | 95] | | | | 287] |
| 4 | 3110 | LFIKHFIIYSIPRYVRDLKI | TPTE [414- | 3 | 4433 | MEDNSALYESTSAHIIEETE | se57-1 [16- |
| | | | 433] | | | | 35] |
| 4 | 3111 | NGQALALPAPSRTLLLALPL | TSP50 [358- | 3 | 4434 | SEPHIKRPMNAFMVWAKDER | SOX-6 [617- |
| | | | 377] | | | | 636] |
| 4 | 3112 | LLGAAMVGAVLTALLAGLVS | TYR [478- | 3 | 4435 | SSEEDKEKEEVAAVKIQAAF | SP17 [106- |
| | | | 497] | | | | 125] |
| 4 | 3113 | EEQCLSAFTVHFSGQFTGTA | WT1 [85-104] | 3 | 4436 | REEAQKMSSLLPTMWLGAQN | SPAG9 [959- |
| | | | | | | | 978] |
| 4 | 3114 | WKVICKSCISQTPGINLDLG | XAGE-1 [36- | 3 | 4437 | ESSTILVVRYRRNVKRTSPE | SPAN-Xc [46- |
| | | | 55]; XAGE- | | | | 65] |
| | | | 1b [36-55] | | | | |
| 4 | 3115 | LHLGSRQKKIRIQLRSQCAT | XAGE-1c [95- | 3 | 4438 | KGVGLPFLPITSSDIDVVES | SPATA19 [14- |
| | | | 114] | | | | 33] |
| 4 | 3116 | CEGGTDVKGKILPKAEHFKM | XAGE-2 [82- | 3 | 4439 | WKKMKYSEKISYVYMKRNYK | SSX-1 [37- |
| | | | 101] | | | | 56] |
| 4 | 3117 | SECGKTFRVSSHLIRHFRIH | ZNF165 [431- | 3 | 4440 | EKIFYVYMKRKYEAMTKLGF | SSX-2 [44- |
| | | | 450] | | | | 63] |
| 5 | 3118 | PTEYERFFALLTPTWKAETT | ACRBP [40- | 3 | 4441 | EWEKMKSSEKIVYVYMKLNY | SSX-4 [36- |
| | | | 59] | | | | 55] |
| 5 | 3119 | KMLEILFELLHVPSVLLADQ | ACTL8 [113- | 3 | 4442 | EWEKMKASEKIIYVYMKRKY | SSX-5 [36- |
| | | | 132] | | | | 55] |
| 5 | 3120 | ESLAVILAQLLSLSMGITYD | ADAM2 [305- | 3 | 4443 | KNKQRQLRLAFEEQLEDLMG | SYCE1 [148- |
| | | | 324] | | | | 167] |
| 5 | 3121 | NITPRMQHDTSHLFTTLGLR | ADAM29 [276- | 3 | 4444 | GADAQYFVYSNESVRPYTPF | TAG-1 [765- |
| | | | 295] | | | | 784] |
| 5 | 3122 | EPTAFRISVTSSCFVQNTLT | BAGE-2 [69- | 3 | 4445 | GDFYVQLYSSEVLEYMNQLS | TDRD1 [277- |
| | | | 88] | | | | 296] |
| 5 | 3123 | EPTPFRISVTSSCFVQNTLT | BAGE-3 [69- | 3 | 4446 | LSQFWEFSETTASTVSTTLH | TEX101 [120- |
| | | | 88] | | | | 139] |
| 5 | 3124 | PSQNQIRNCYQNFLDYHRCL | COX6B2 [24- | 3 | 4447 | KSDIYSFSMIMQEILTDDIP | TEX14 [436- |
| | | | 43] | | | | 455] |
| 5 | 3125 | MALLPVLFLVTVLLPSLPAE | CRISP2 [1- | 3 | 4448 | LKKSKYFISTYIDFVPYIAS | TEX15 [2172- |
| | | | 20] | | | | 2191] |
| 5 | 3126 | TQLVKWMLGCYDALQKKYLR | CT46 [76-95] | 3 | 4449 | DQVDWSRLLRDAGLVKMSRK | TRAG-3 [52- |
| | | | | | | | 71] |
| 5 | 3127 | MVHSLLHRLSHNDHILIENR | CT47 [126- | 3 | 4450 | MANERISMQNLEALLVANRD | TSGA10 [557- |
| | | | 145] | | | | 576] |
| 5 | 3128 | LTSFPTFTSVGVLIVLVLCS | CTAGE1 [41- | 3 | 4451 | LVNITEYRASHSTPIEWYPD | TSPY1 [221- |
| | | | 60] | | | | 240] |
| 5 | 3129 | RYCSAVFHERYALIQHQKTH | CTCFL [461- | 3 | 4452 | AVRRGRRGKKGAATKMAAVT | VCX [44-63] |
| | | | 480] | | | | |
| 5 | 3130 | AKNLMNAVVLTVKASYVAST | CTNNA2 [869- | 3 | 4453 | QLKVGILHLGSRQKKIRIQL | XAGE-1d [10- |
| | | | 888] | | | | 29]; XAGE- |
| | | | | | | | 1 [10-29]; |
| | | | | | | | XAGE-1b [10- |
| | | | | | | | 29]; XAGE- |
| | | | | | | | 1c [89-108] |
| 5 | 3131 | VMVESIQFCFIWRAISITPV | CXorf48 [220- | 3 | 4454 | TPDQKREDDQGAAEIQVPDL | XAGE-2 [47- |
| | | | 239] | | | | 66] |
| 5 | 3132 | MSEVEAAAGATAVPAATVPA | DBPC [1-20] | 4 | 4455 | EVRAGAFEHLPSLRQLDLSH | 5T4 [132-151] |
| 5 | 3133 | HAMSSQYRMHSYYPPPSYLG | DMRT1 [282- | 4 | 4456 | PHNFRVYSYSGTGIMKPLDQ | ADAM2 [67- |
| | | | 301] | | | | 86] |
| 5 | 3134 | AVVAGIVVLVISRKKRMAKY | EpCAM [278- | 4 | 4457 | QKLGEYYLQNAFLVAYTKKA | AFP [420-439] |
| | | | 297] | | | | |
| 5 | 3135 | AARACFALLWGCALAAAAAA | EPHA2 [5-24] | 4 | 4458 | DLRSVFFNFIRNLLSETIFK | AKAP-3 [589- |
| | | | | | | | 608] |
| 5 | 3136 | GSVASYILASHNGISYKDLD | FAM46D [65- | 4 | 4459 | LDSQKMDMSNIVLMLIQKLL | AKAP-4 [619- |
| | | | 84] | | | | 638] |
| 5 | 3137 | EQGATWRHRETLIIAVLVSA | FATE1 [153- | 4 | 4460 | QNKPLYFADRLYDSMKGKGT | ANXA2 [264- |
| | | | 172] | | | | 283] |
| 5 | 3138 | SCRKLFYFKIWAFLDVSFVE | FBXO39 [364- | 4 | 4461 | TALDVHFVSTLEPLSNAVKR | BAGE-2 [37- |
| | | | 383] | | | | 56]; BAGE- |
| | | | | | | | 3 [37-56] |
| 5 | 3139 | LEALLNFFFPTTCNLRENQV | FMR1NB [122- | 4 | 4462 | SEILKEMLAKKHFSYAWPFY | BRDT [277- |
| | | | 141] | | | | 296] |
| 5 | 3140 | AAINSHITLELYTSYLYLSM | FTHL17 [19- | 4 | 4463 | AYFQELTMYRGNTTMDIKDL | CABYR [39- |
| | | | 38] | | | | 58] |
| 5 | 3141 | GPVTAQVVLQAAIPLTSCGS | gp100 [284- | 4 | 4464 | NSPTSLLIYKDAPAFNEKAS | CCDC62 [600- |
| | | | 303] | | | | 619] |
| 5 | 3142 | NVARCWTYETAVALDTEIPN | HDAC1 [307- | 4 | 4465 | EEGNEAANFDLAVVARRYPA | CT47 [97-116] |
| | | | 326] | | | | |
| 5 | 3143 | ASGFCYVNDIVIGILELLKY | HDAC3 [141- | 4 | 4466 | SEAVELQDMSLLSIQQQEGV | CTCFL [89- |
| | | | 160] | | | | 108] |
| 5 | 3144 | KPGIGVLLDTNYNLFYWYEG | IL13RA2 [158- | 4 | 4467 | SSDSSMLDSATSLIQAAKNL | CTNNA2 [853- |
| | | | 177] | | | | 872] |
| 5 | 3145 | LDLLKSEYPVIQLLALKTLG | KU-CT- | 4 | 4468 | SNSIYFSIAIVSEDFVPYKG | CXorf48 [129- |
| | | | 1 [197-216] | | | | 148] |
| 5 | 3146 | AQGDGCRGVAFNVMFSAPHI | Lage-1 [191- | 4 | 4469 | PHSIARQKRILVNLSMVENK | CXorf61 [71- |
| | | | 210] | | | | 90] |
| 5 | 3147 | CTYKIQRLMLKSLTYPERPP | LIPI [436- | 4 | 4470 | DTKEDVFVHQTAIKRNNPRK | DBPC [114- |
| | | | 455] | | | | 133] |
| 5 | 3148 | GLVCVQAATSSSSPLVLGTL | MAGE-A1 [24- | 4 | 4471 | GSEWSVYAVGSQAHVSFLDP | DCAF12 [305- |
| | | | 43] | | | | 324] |
| 5 | 3149 | AAISRKMVELVHFLLLKYRA | MAGE- | 4 | 4472 | GIMGQFSHHNIIRLEGVISK | EPHA2 [665- |
| | | | A2 [107-126] | | | | 684] |
| 5 | 3150 | KTGLLIIVLGTIAMEGDSAS | MAGE- | 4 | 4473 | RGPTQSVGPTIQDYLNRPRP | FAM133A [20- |
| | | | [199-218] | | | | 39] |
| 5 | 3151 | GGASSSISVYYTLWSQFDEG | MAGE-A9 [63- | 4 | 4474 | SAKRVWNMTATRPKKMGSQL | FATE1 [63- |
| | | | 82] | | | | 82] |
| 5 | 3152 | SWDFPRRKLLMPLLGVIFLN | MAGE- | 4 | 4475 | WRNSIRSSFISSLSFFLKKM | FBXO39 [141- |
| | | | B2 [195-214] | | | | 160] |
| 5 | 3153 | PGLYPHLYEDALIDEVERAL | MAGE- | 4 | 4476 | RESLKMRVSKPFGMLMLSIW | FMR1NB [58- |
| | | | 6 [384-403] | | | | 77] |
| 5 | 3154 | GIGILTVILGVLLLIGCWYC | MART1 [29- | 4 | 4477 | EEEDTFSSVFHTQIPPEEYE | FSIP1 [191- |
| | | | 48] | | | | 210] |
| 5 | 3155 | NSYPYYPYLYCVGSWSPAFW | NKX3.1 [215- | 4 | 4478 | LSMAFYFNRDDVALENFFRY | FTHL17 [36- |
| | | | 234] | | | | 55] |
| 5 | 3156 | AIFYCLFEMQDPAFVKQAVN | NLRP4 [552- | 4 | 4479 | SMELKFLIIQNAAVFQEAFE | Glypican- |
| | | | 571] | | | | 3 [97-116] |
| 5 | 3157 | KDYTCLLSSTWQELILLSSL | NR6A1 [314- | 4 | 4480 | AHSSSAFTITDQVPFSVSVS | gp100 [201- |
| | | | 333] | | | | 220] |
| 5 | 3158 | IDIILNRRNCMAATLKIIER | NXF2 [248- | 4 | 4481 | GAGKGKYYAVNYPLRDGIDD | HDAC1 [215- |
| | | | 267] | | | | 234] |
| 5 | 3159 | MYGTSLYQFIPLTFVMPNDY | NYC- | 4 | 4482 | RGHYERILNPYNLFLSGDSL | JARID1B [172- |
| | | | TSPG [169- | | | | 1911] |
| | | | 188] | | | | |
| 5 | 3160 | KRELAKLRRTTNRILASSCC | ODF1 [102- | 4 | 4483 | QHIKQLSRFAGASIKIAPAE | KOC1 [426- |
| | | | 121] | | | | 445] |
| 5 | 3161 | SSPGPKYLIPPTTGFMKHTP | ODF3 [24-43] | 4 | 4484 | AELVRRILSRDAAPLPRPGA | Lage-1 [102- |
| | | | | | | | 121] |
| 5 | 3162 | THSFRWMAQVLASELSLVAF | ODF4 [73-92] | 4 | 4485 | NLDSARFRYLIGEKLGVHPT | LDHC [164- |
| | | | | | | | 183] |
| 5 | 3163 | LRGHFCLSSDKMVCYLLKTK | OIP5 [158- | 4 | 4486 | PDKTMMDGSFSFKLLNQLGM | LIPI [367- |
| | | | 177] | | | | 386] |
| 5 | 3164 | GNVCILRTQLLQQLYTSKAV | PASD1 [171- | 4 | 4487 | FPEIFGKASESLQLVFGIDV | MAGE- |
| | | | 190] | | | | A1 [140-159] |
| 5 | 3165 | LWKKRWFVLSDLCLFYYRDE | PEPP2 [186- | 4 | 4488 | QDFFPVIFSKASEYLQLVFG | MAGE- |
| | | | 205] | | | | A2 [144-163] |
| 5 | 3166 | VTGNVTAFDGSILYLPVKLQ | PIWIL2 [267- | 4 | 4489 | NKVDELAHFLLRKYRAKELV | MAGE- |
| | | | 286] | | | | A4 [112-131] |
| 5 | 3167 | RELPLLNAMPLHSWLILYSR | PIWIL3 [497- | 4 | 4490 | TTEEQEAVSSSSPLVPGTLG | MAGE-A5 [32- |
| | | | 516] | | | | 51] |
| 5 | 3168 | DVSYKVLRNETVLEFMTALC | PIWIL4 [262- | 4 | 4491 | EEQKAASSSSTLIMGTLEEV | MAGE-A8 [34- |
| | | | 281] | | | | 53] |
| 5 | 3169 | GTPSKFVIPVSCAAPQKSPW | PLAC1 [104- | 4 | 4492 | VIKNYKRYFPVIFGKASEFM | MAGE- |
| | | | 123] | | | | A9 [138-157] |
| 5 | 3170 | RAVGLLTVISKGCSLNCVDD | PSCA [45-64] | 4 | 4493 | DNPSGHTYTLVSKLNLTNDG | MAGE- |
| | | | | | | | B1 [168-187] |
| 5 | 3171 | PSNALSTVLPGLAYLATADM | SAGE1 [359- | 4 | 4494 | PLTRKSGSLVQFLLYKYKIK | MAGE- |
| | | | 378] | | | | B2 [110-129] |
| 5 | 3172 | GNAKYANMWLEYYNLERAHG | SART3 [503- | 4 | 4495 | SLTRKTKMLVQFLLYKYKMK | MAGE- |
| | | | 522] | | | | B4 [108-127] |
| 5 | 3173 | VPDKLRSLALGVSYVILRIF | SLCO6A1 [607- | 4 | 4496 | PVSPSFSSTLVSLFQSSPER | MAGE- |
| | | | 626] | | | | C1 [271-290] |
| 5 | 3174 | MSIPFSNTHYRIPQGFGNLL | SP17 [1-20] | 4 | 4497 | VFSPSSFSTSSSLILGGPEE | MAGE-C2 [49- |
| | | | | | | | 68] |
| 5 | 3175 | IEQVRRSISRLTDVSAQDFS | SPATA19 [140- | 4 | 4498 | DDPQKFAMELSIIYKYSPFK | MORC1 [159- |
| | | | 159] | | | | 178] |
| 5 | 3176 | MCIYKYGSMSMSFEAHHLTV | SPO11 [286- | 4 | 4499 | STEKNAVSMTSSVLSSHSPG | MUC-1 [48- |
| | | | 305] | | | | 67] |
| 5 | 3177 | EHAWTHRLRERKQLVIYEEI | SSX-2 [161- | 4 | 4500 | VQPTQKQQKHRLFHWQANSE | NA17-A [45- |
| | | | 180] | | | | 64] |
| 5 | 3178 | HKDLWDFHMPERLAKEICAL | SYCE1 [169- | 4 | 4501 | IWFQNRRYKTKRKQLSSELG | NKX3.1 [170- |
| | | | 188] | | | | 189] |
| 5 | 3179 | PDEVENQFILRLPLEHACTV | TAF7L [95- | 4 | 4502 | MKAINFLNQDIRGLTSASQL | NR6A1 [392- |
| | | | 114] | | | | 411] |
| 5 | 3180 | AQDAGVYQCLASNPVGTVVS | TAG-1 [103- | 4 | 4503 | EDSERLMEQQGALLKRLAEA | ODF2 [281- |
| | | | 122] | | | | 300] |
| 5 | 3181 | CNLEPLVSRDPPASASLFQD | TAG-2a [63- | 4 | 4504 | EKSTKYVFDSAPSHSISART | ODF3 [109- |
| | | | 82] | | | | 128] |
| 5 | 3182 | TIQANVLEIISPNLFYALPK | TDRD1 [941- | 4 | 4505 | SRVTNNVVLEAPFLVGIEGS | OIP5 [107- |
| | | | 960] | | | | 126] |
| 5 | 3183 | EEAITIVQHSSPPGLIVTSY | TEX101 [86- | 4 | 4506 | VYQKRLMDEAKILKSLHHPN | PBK [77-96] |
| | | | 105] | | | | |
| 5 | 3184 | LLKAGVISAQNIYSFGFGKA | TEX14 [194- | 4 | 4507 | PTPESSTIASYVTLRKTKKM | PEPP2 [856- |
| | | | 213] | | | | 875] |
| 5 | 3185 | TSSDRTINLLEVLPWPTEWI | TMEM31 [62- | 4 | 4508 | QWALYQYHIDYNPLMEARRL | PIWIL1 [126- |
| | | | 81] | | | | 145] |
| 5 | 3186 | YDNCSFYFWLHTSFIENNRL | TPTE [489- | 4 | 4509 | RQFVEFTIKEAARFKKVVLI | SAGE1 [860- |
| | | | 508] | | | | 879] |
| 5 | 3187 | VQELRRQNYSSNAYHMSSTM | TSGA10 [645- | 4 | 4510 | DKKTQTFLLETPEIYWKLDS | SCP-1 [793- |
| | | | 664] | | | | 812] |
| 5 | 3188 | ECDNFYHNFTKIPTLVQIIK | TSP50 [272- | 4 | 4511 | QNEFKKEMAMLQKKIMMETQ | SCP3a [201- |
| | | | 291] | | | | 220] |
| 5 | 3189 | ECMTWNQMNLGATLKGVAAG | WT1 [234- | 4 | 4512 | RSIFKPFIFVDDVKLVPKTQ | SCRN1 [302- |
| | | | 253] | | | | 321] |
| 5 | 3190 | NECGKAFRHSSKLARHQRIH | ZNF165 [347- | 4 | 4513 | LQFVIHSQHQNLRSVIQEME | se57-1 [246- |
| | | | 366] | | | | 265] |
| 6 | 3191 | LYGGLHMDFWCARLATKGCE | ACRBP [433- | 4 | 4514 | GSSLDILSSLNSPALFGDQD | SOX-6 [475- |
| | | | 452] | | | | 494] |
| 6 | 3192 | VVDSGYGLTRVQPFHQGRPL | ACTL8 [147- | 4 | 4515 | GSKVEDRFYNNHAFEEQEPP | SP17 [61-80] |
| | | | 166] | | | | |
| 6 | 3193 | KTSYLVLRPHDVAFLLVYRE | ADAM2 [254- | 4 | 4516 | RDAVKFFVAVPGQVISPQSS | SPAG9 [1223- |
| | | | 273] | | | | 1242] |
| 6 | 3194 | FTTLGLRGLSGIGAFRGMCT | ADAM29 [289- | 4 | 4517 | TILVVRYRRNVKRTSPEELL | SPAN-Xc [49- |
| | | | 308] | | | | 68] |
| 6 | 3195 | ISLADLATIFFAQFVQEATY | AFP [41-60] | 4 | 4518 | EWEKMKVSEKIVYVYMKRKY | SSX-3 [36- |
| | | | | | | | 55] |
| 6 | 3196 | FLNLVQCIQNKPLYFADRLY | ANXA2 [256- | 4 | 4519 | TFGSLQRIFPKIMPKKPAEE | SSX-4 [100- |
| | | | 275] | | | | 119] |
| 6 | 3197 | ALQIVYGIRLEHFYMMPVNS | CDCA1 [52- | 4 | 4520 | QRQKDLIMKVENLTLKNHFQ | TAF7L [413- |
| | | | 71] | | | | 432] |
| 6 | 3198 | KSPNAVVGHYTQLVWYSTYQ | CRISP2 [128- | 4 | 4521 | AEDTRLFAPSIKARFPAETY | TAG-1 [231- |
| | | | 147] | | | | 250] |
| 6 | 3199 | DKTEKVAVDPETVFKRPREC | CT45 [3-22] | 4 | 4522 | YIEIVMVSETIHFLKNSIAK | TEX15 [2062- |
| | | | | | | | 2081] |
| 6 | 3200 | NFYLPQNYIDQFLLTSFPTF | CTAGE1 [28- | 4 | 4523 | MWMGLIQLVEGVKRKDQGFL | TRAG-3 [1- |
| | | | 47] | | | | 20] |
| 6 | 3201 | PQRQDRFYSNCARLSGPAEL | CTAGE2 [590- | 4 | 4524 | LNAERSYKSQISTLHKSVVK | TSGA10 [470- |
| | | | 609] | | | | 489] |
| 6 | 3202 | AAVVGFFAVLFFLWRSFRSV | CTAGE5 [47- | 4 | 4525 | GEEAVLLLDDIMAEVEVVAE | TSPY1 [60- |
| | | | 66] | | | | 79] |
| 6 | 3203 | CHLCLKTFRTVTLLRNHVNT | CTCFL [287- | 5 | 4526 | NSLVSLTYVSFRNLTHLESL | 5T4 [244-263] |
| | | | 306] | | | | |
| 6 | 3204 | VFWKYRRFQRNTGEMSSNST | CXorf61 [17- | 5 | 4527 | NFGTRTFQAITVTKLSQKFT | AFP [229-248] |
| | | | 36] | | | | |
| 6 | 3205 | VSRVPVYAHITHKALKDIPK | DCAF12 [226- | 5 | 4528 | SVVSDMMVSIMKTLKIQVKD | AKAP-3 [291- |
| | | | 245] | | | | 310] |
| 6 | 3206 | CPDCAKRNKKMMKRLMTVEK | DPPA2 [279- | 5 | 4529 | SDLQKYALGFQHALSPSTST | AKAP-4 [116- |
| | | | 298] | | | | 135] |
| 6 | 3207 | DVWSFGIVMWEVMTYGERPY | EPHA2 [799- | 5 | 4530 | RKYGKSLYYYIQQDTKGDYQ | ANXA2 [309- |
| | | | 818] | | | | 328] |
| 6 | 3208 | IKNLERYMCSRFFIDFPHIE | FAM46D [255- | 5 | 4531 | LKDLWKHSFSWPFQRPVDAV | BRDT [40-59] |
| | | | 274] | | | | |
| 6 | 3209 | SMRPTLIDLLPTFRHTLQKL | FBXO39 [324- | 5 | 4532 | DQSDVLMVDVATSMPVVIKE | CABYR [189- |
| | | | 343] | | | | 208] |
| 6 | 3210 | SSGTTSFKCFAPFRDVPKQM | FMR1NB [165- | 5 | 4533 | KITKQQVFIDVINKLKENVE | CAGE1 [343- |
| | | | 184] | | | | 362] |
| 6 | 3211 | DPNVACRRLMTPIMYAARDG | GASZ [141- | 5 | 4534 | FSNKGKTLVIQYTVKHEQKM | CALR3 [84- |
| | | | 160] | | | | 103] |
| 6 | 3212 | ADQPRWVSHHHPAVLNGQHP | GATA-3 [5- | 5 | 4535 | ELHKRTEIIRSLTKKVKALE | CCDC62 [78- |
| | | | 24] | | | | 97] |
| 6 | 3213 | HSPLKLLTSMAISVVCFFFL | Glypican- | 5 | 4536 | AKRTSRFLSGIINFIHFREA | CDCA1 [114- |
| | | | 3 [559-578] | | | | 133] |
| 6 | 3214 | SSGTLISRALVVTHTYLEPG | gp100 [265- | 5 | 4537 | AGDSLIAGSAMSKAKKLMTG | CT45 [42-61] |
| | | | 284] | | | | |
| 6 | 3215 | MSKVMEMFQPSAVVLQCGSD | HDAC1 [245- | 5 | 4538 | PSSETRAFLSPPTLLEGPLR | CTAGE5 [527- |
| | | | 264] | | | | 546] |
| 6 | 3216 | GVEEAFYTTDRVMTVSFHKY | HDAC2 [183- | 5 | 4539 | DLSRDILNNFPHSIARQKRI | CXorf61 [61- |
| | | | 202]; | | | | 80] |
| | | | HDAC1 [182- | | | | |
| | | | 201] | | | | |
| 6 | 3217 | GVQEAFYLTDRVMTVSFHKY | HDAC3 [176- | 5 | 4540 | EVRLQNETSYSRVLHGYAAQ | DCAF12 [54- |
| | | | 195] | | | | 73] |
| 6 | 3218 | PPHTHSYTISHATLERIGAV | IGFS11 [398- | 5 | 4541 | GLTGLQSLLQGFSRLFLKGN | DKKL1 [44- |
| | | | 417] | | | | 63] |
| 6 | 3219 | FQLQNIVKPLPPVYLTFTRE | IL13RA2 [230- | 5 | 4542 | VDDEESVILTLVPVKDDANM | DPPA2 [18- |
| | | | 249] | | | | 37] |
| 6 | 3220 | SETETVHLFIPALSVGAIIG | KOC1 [403- | 5 | 4543 | KQLENKKTGSKALAEFEEKM | FAM133A [46- |
| | | | 422] | | | | 65] |
| 6 | 3221 | HEFASLCLANMSAEYTSKVQ | KU-CT- | 5 | 4544 | PVFPQLSRSIISKLLNESET | FSIP1 [439- |
| | | | 1 [126-145] | | | | 458] |
| 6 | 3222 | GTYPFCTYYFVLSIIVPDKT | LIPI [351- | 5 | 4545 | GWEIGYLDRTSQKLKRLLPI | GASZ [24-43] |
| | | | 370] | | | | |
| 6 | 3223 | CILESLFRAVITKKVADLVG | MAGE-A1 [92- | 5 | 4546 | FPKNSSFNPAALSRHMSSLS | GATA-3 [386- |
| | | | 111] | | | | 405] |
| 6 | 3224 | EKVTDLVQFLLFKYQMKEPI | MAGE- | 5 | 4547 | KHINQLLRTMSMPKGRVLDK | Glypican- |
| | | | A10 [136- | | | | 3 [467-486] |
| | | | 155] | | | | |
| 6 | 3225 | EVVEVVRIGHLYILVTCLGL | MAGE- | 5 | 4548 | DRQTVMTSATWPHSVHRLAQ | HAGE [420- |
| | | | A12 [165- | | | | 439] |
| | | | 184] | | | | |
| 6 | 3226 | SRKVAELVHFLLLKYRAREP | MAGE- | 5 | 4549 | GAGKGKYYAVNFPMRDGIDD | HDAC2 [216- |
| | | | A3 [110-129] | | | | 235] |
| 6 | 3227 | EFLWGPRALAETSYVKVLEH | MAGE- | 5 | 4550 | PNHPSRKKVNFLDMSLDDII | HOM-TES- |
| | | | A4 [271- | | | | 85 [15-34] |
| | | | 290]; MAGE- | | | | |
| | | | A8 [273-292] | | | | |
| 6 | 3228 | GLDTQEEALGLVGVQAATTE | MAGE-A5 [15- | 5 | 4551 | SSDNNTLTSSNAYNSRYWSN | IGFS11 [305- |
| | | | 34] | | | | 324] |
| 6 | 3229 | EALKLKVAELVHFLLHKYRV | MAGE- | 5 | 4552 | TLDDLYPMMNALKLRAESYN | JARID1B [737- |
| | | | A9 [106-125] | | | | 756] |
| 6 | 3230 | EVNPTTHSYILVSMLGPNDG | MAGE- | 5 | 4553 | EAQDIKFTEEIPLKILAHNN | KOC1 [267- |
| | | | B4 [168-187] | | | | 286] |
| 6 | 3231 | DPDDSYVFVNTLDLTSEGCL | MAGE- | 5 | 4554 | TSGKDYSVSANSRIVIVTAG | LDHC [78-97] |
| | | | C1 [969-988] | | | | |
| 6 | 3232 | EKNCEPVVPNAPPAYEKLSA | MART1 [90- | 5 | 4555 | PSKGRRWAARAPSTRITYGT | LEMD1 [111- |
| | | | 109] | | | | 130] |
| 6 | 3233 | IELERKFSHQKYLSAPERAH | NKX3.1 [137- | 5 | 4556 | DDTTAMASASSSATGSFSYP | MAGE-A10 [349-368] |
| | | | 156] | | | | |
| 6 | 3234 | LIEDGYAVTQAELFALLCRL | NR6A1 [271- | 5 | 4557 | EEQEAAFFSSTLNVGTLEEL | MAGE-A11 [144-163] |
| | | | 290] | | | | |
| 6 | 3235 | LKPLVFRVDETTPAVVQSVL | NYC- | 5 | 4558 | DGREDSVFAHPRKLLTQDLV | MAGE-A12 [232-251] |
| | | | TSPG [84- | | | | |
| | | | 103] | | | | |
| 6 | 3236 | NILTIRLTAADHRQLQLSIS | NY-ESO- | 5 | 4559 | PDLESEFQAALSRKVAELVH | MAGE-A3 [99- |
| | | | 1 [131-150] | | | | 118] |
| 6 | 3237 | VDRELRQLRCIDEFSTRCLC | ODF1 [18-37] | 5 | 4560 | AIPTAIDFTLWRQSIKGSSN | MAGE-A5 [70- |
| | | | | | | | 89] |
| 6 | 3238 | YDKKIDSLMNAVGCLKSEVK | ODF2 [123- | 5 | 4561 | FPVIFSKASDSLQLVFGIEL | MAGE- |
| | | | 142] | | | | A6 [147-166] |
| 6 | 3239 | DSTPGPAAYMLPMVMGPNTV | ODF3 [134- | 5 | 4562 | RTTATTFRARSRAPFSRSSH | MAGE- |
| | | | 153] | | | | B1 [326-345] |
| 6 | 3240 | SIPIGWSYFIGWLVLILYFT | ODF4 [176- | 5 | 4563 | TVPSAFQFWYEEALRDEEER | MAGE- |
| | | | 195] | | | | B3 [298-317] |
| 6 | 3241 | QVTLQLLDGFMITLSTDGVI | PASD1 [35- | 5 | 4564 | NSDPPRYQFLWGPRAHAETS | MAGE- |
| | | | 54] | | | | B4 [262-281] |
| 6 | 3242 | PICNDHYRSVYQKRLMDEAK | PBK [68-87] | 5 | 4565 | ISRYTGYFPVIFRKAREFIE | MAGE- |
| | | | | | | | C1 [939-958] |
| 6 | 3243 | PDHIQRLTYKLCHIYYNWPG | PIWIL1 [805- | 5 | 4566 | DSESSFTYTLDEKVAELVEF | MAGE- |
| | | | 824] | | | | C2 [132-151] |
| 6 | 3244 | KYTRPTLQMGMSCLLVFKVI | PIWIL3 [561- | 5 | 4567 | STTHSFLFGALAELLDNARD | MORC1 [22- |
| | | | 580] | | | | 41] |
| 6 | 3245 | LDQLLRHVMNPLETLSITNC | PRAME [313- | 5 | 4568 | SSKKTYSLRSQASIIGVNLA | MPHOSPH1 [1703- |
| | | | 332] | | | | 1722] |
| 6 | 3246 | NCVDDSQDYYVGKKNITCCD | PSCA [60-79] | 5 | 4569 | LQRDISEMFLQIYKQGGFLG | MUC-1 [1069- |
| | | | | | | | 1088] |
| 6 | 3247 | YELVEKFYDPMFKYHLTVAQ | PSMA [559- | 5 | 4570 | HRLFHWQANSERADIPASLR | NA17-A [54- |
| | | | 578] | | | | 73] |
| 6 | 3248 | SRTDKVLSTAPPQLVHMAAA | SAGE1 [122- | 5 | 4571 | EKNEEIFNYNNHLKNRIYQY | NY-BR- |
| | | | 141] | | | | 1 [1312- |
| | | | | | | | 1331] |
| 6 | 3249 | GKKHRYLRLLPEALIRFGGF | SLCO6A1 [45- | 5 | 4572 | AMPFATPMEAELARRSLAQD | NY-ESO- |
| | | | 64] | | | | 1 [93-112] |
| 6 | 3250 | TTGTGVVYPGAITMATTTPS | SOX-6 [736- | 5 | 4573 | LELEIIVLNDRVTDLVNQQQ | ODF2 [458- |
| | | | 755] | | | | 477] |
| 6 | 3251 | AEAAGKYSAAIALLEPAGSE | SPAG1 [462- | 5 | 4574 | IILKVALNMARGLKYLHQEK | PBK [142-161] |
| | | | 481] | | | | |
| 6 | 3252 | NILDSFTVCNSHVLCIASVP | SPAG9 [780- | 5 | 4575 | VTPAMGMQMRKAIMIEVDDR | PIWIL1 [518- |
| | | | 799] | | | | 537] |
| 6 | 3253 | RLRERKQLVIYEEISDPEED | SSX-2 [167- | 5 | 4576 | ETFKAVLDGLDVLLAQEVRP | PRAME [95- |
| | | | 186] | | | | 114] |
| 6 | 3254 | PPAWQPFLKDHRISTFKNWP | Survivin [7- | 5 | 4577 | KNSVKTDLMKAPMVIMDWEE | PRSS55 [202- |
| | | | 26] | | | | 221] |
| 6 | 3255 | QECKERISALNLQIEEEKNK | SYCE1 [131- | 5 | 4578 | STNEVTRIYNVIGTLRGAVE | PSMA [348- |
| | | | 150] | | | | 367] |
| 6 | 3256 | REEKCVWKHGITPPLKNVRK | TAF7L [212- | 5 | 4579 | VVRLSSYSSPTLQSVLGSGT | RAGE-1 [357- |
| | | | 231] | | | | 376] |
| 6 | 3257 | RLLLRLECNVVIIAHCNLEP | TAG-2a [48- | 5 | 4580 | MTPTIRKTQKIVIKKREPLN | RCAS1 [98- |
| | | | 67] | | | | 117] |
| 6 | 3258 | TSGLELYCQKGLSMTVEADP | TEX101 [21- | 5 | 4581 | HDLEIQLTAITTSEQYYSKE | SCP-1 [479- |
| | | | 40] | | | | 498] |
| 6 | 3259 | IVHLLLQISDALRYLHFQGF | TEX14 [353- | 5 | 4582 | KILQQSRIVQSQRLKTIKQL | SCP3a [159- |
| | | | 372] | | | | 178] |
| 6 | 3260 | TISFFLCQMLYNRRKILQLK | TEX15 [2250- | 5 | 4583 | RRHELYKAHEWARAIIESDQ | SCRN1 [343- |
| | | | 2269] | | | | 362] |
| 6 | 3261 | FYKFFLPTILSLSFFILLVL | TMEM31 [135- | 5 | 4584 | IEETEYVKKIRTTLQKIRTQ | se57-1 [31- |
| | | | 154] | | | | 50] |
| 6 | 3262 | YIPLEYRSISLAIALFFLMD | TPTE [121- | 5 | 4585 | IGEYKQLMRSRRQEMRQFFT | SOX-6 [707- |
| | | | 140] | | | | 726] |
| 6 | 3263 | FYHKTNIKMRCEFLACWPAF | TRAG-3 [24- | 5 | 4586 | SMKPLLRRAMAYETLEQYGK | SPAG1 [520- |
| | | | 43] | | | | 539] |
| 6 | 3264 | TRELCIKLDSSKELLNRQLV | TSGA10 [511- | 5 | 4587 | EDGRVQAFGWSLPQKYKQVT | SPAG9 [639- |
| | | | 530] | | | | 658] |
| 6 | 3265 | GEPLVCSMEGTWYLVGLVSW | TSP50 [311- | 5 | 4588 | HRLRERKQLVIYEEISDPEE | SSX-1 [166- |
| | | | 330] | | | | 185] |
| 6 | 3266 | LDRRGAVIQSVPGFWANVIA | TSPY1 [145- | 5 | 4589 | AMTKLGFKAILPSFMRNKRV | SSX-3 [57- |
| | | | 164] | | | | 76] |
| 6 | 3267 | LCGAQYRIHTHGVFRGIQDV | WT1 [281- | 5 | 4590 | GKHAWTHRLRERKQLVVYEE | SSX-4 [160- |
| | | | 300] | | | | 179] |
| 6 | 3268 | MRCHAHGPSCLVTAITREEG | XAGE-1c [1- | 5 | 4591 | ATLPPFMRNKRVADFQGNDF | SSX-5 [65- |
| | | | 20] | | | | 84] |
| 7 | 3269 | YVRNLFLTGNQLAVLPAGAF | 5T4 [92-111] | 5 | 4592 | TNNKKKEFEETAKKVRRAIE | Survivin [117- |
| | | | | | | | 136] |
| 7 | 3270 | NPGSLLQLPHTEALLVLCYS | ACRBP [312- | 5 | 4593 | DQMWRQFTDTNLAFNARISE | TEKT5 [329- |
| | | | 331] | | | | 348] |
| 7 | 3271 | QLQMSLYASGLLTGVVVDSG | ACTL8 [132- | 5 | 4594 | VTELEYNYNQFSTLLKNVMS | TEX15 [2198- |
| | | | 151] | | | | 2217] |
| 7 | 3272 | DRKVICFVDVSTLNVEDKDY | AKAP-4 [38- | 5 | 4595 | MPMSEVSQVSRAAQMAVPSS | THEG [247- |
| | | | 57] | | | | 266] |
| 7 | 3273 | EPLSNAVKRNVPRCIIILVL | BAGE-2 [48- | 5 | 4596 | DAGLVKMSRKPRASSPLSNN | TRAG-3 [62- |
| | | | 67]; BAGE- | | | | 81] |
| | | | 3 [48-67] | | | | |
| 7 | 3274 | ISISSLRQFETVCKFHWVEA | CAGE1 [113- | 5 | 4597 | FWANVIANHPQMSALITDED | TSPY1 [158- |
| | | | 132] | | | | 177] |
| 7 | 3275 | THLYTLILRPDLSYDVKIDG | CALR3 [169- | 5 | 4598 | ADASQSSMHNALHIYMNGTM | TYR [355-374] |
| | | | 188] | | | | |
| 7 | 3276 | SSAIQSWYDEILDFVYGVGP | CRISP2 [108- | 5 | 4599 | WRGRSTYRPRPRRSLQPPEL | XAGE-2 [3- |
| | | | 127] | | | | 22] |
| 7 | 3277 | HILIENRQLSRLMVGPHAAA | CT47 [139- | 6 | 4600 | FSGSNASVSAPSPLVELILN | 5T4 [162-181] |
| | | | 158] | | | | |
| 7 | 3278 | LIVLVLCSAFLLLWQGEGVN | CTAGE1 [53- | 6 | 4601 | NRNVNFAMKSETKLREKMYS | AKAP-3 [417- |
| | | | 72] | | | | 436] |
| 7 | 3279 | IGCVTSINEDNIYISNSIYF | CXorf48 [115- | 6 | 4602 | SKGLMVYANQVASDMMVSLM | AKAP-4 [330- |
| | | | 134] | | | | 349] |
| 7 | 3280 | NPNSLAIYRLPTLDPVCVGD | DCAF12 [161- | 6 | 4603 | VGTIDMTLQSDIMTMFENNF | BRDT [927- |
| | | | 180] | | | | 946] |
| 7 | 3281 | VSVEAFLMQASGVRWCVVHG | DPPA2 [209- | 6 | 4604 | SKPRLVVPYGLKTLLEGISR | CABYR [4-23] |
| | | | 228] | | | | |
| 7 | 3282 | NTVICSKLAAKCLVMKAEMN | EpCAM [55- | 6 | 4605 | NNIENYSTNALIQPVDTISI | CAGE1 [96- |
| | | | 74] | | | | 115] |
| 7 | 3283 | VCRKWNQMMYSAELWRYRTI | FBXO39 [42- | 6 | 4606 | ESGSIEYDWNLTSLKKETSP | CALR3 [192- |
| | | | 61] | | | | 211] |
| 7 | 3284 | RNRRSHRAMRVAHLELATYE | FMR1NB [11- | 6 | 4607 | DKELNDMVAVHQQQLLSWEE | CCDC62 [41- |
| | | | 30] | | | | 60] |
| 7 | 3285 | NKHHEIFNLLSFTLNPLEGK | GASZ [225- | 6 | 4608 | VGEVSTPFHIFKVKVTTERE | CT46 [212- |
| | | | 244] | | | | 231] |
| 7 | 3286 | ACGLYHKMNGQNRPLIKPKR | GATA-3 [287- | 6 | 4609 | LGEEEGEQAAGLAAVPRGGS | CT47 [61-80] |
| | | | 306] | | | | |
| 7 | 3287 | VNGMYRIYDMENVLLGLFST | Glypican- | 6 | 4610 | RGSQKKHISPVQALSEFKAM | CTNNA2 [931- |
| | | | 3 [307-326] | | | | 950] |
| 7 | 3288 | LSIGTGRAMLGTHTMEVTVY | gp100 [170- | 6 | 4611 | IDESIYFSSDVVTGNVPLKV | CXorf48 [53- |
| | | | 189] | | | | 72] |
| 7 | 3289 | TEEEKWSHMQTFLQSMSSTD | HAGE [469- | 6 | 4612 | HQTAIKRNNPRKFLRSVGDG | DBPC [122- |
| | | | 488] | | | | 141] |
| 7 | 3290 | AILELLKYHQRVLYIDIDIH | HDAC1 [159- | 6 | 4613 | SRLSPRKTHLLYILRPSRQL | DKKL1 [223- |
| | | | 178]; | | | | 242] |
| | | | HDAC2 [160- | | | | |
| | | | 179] | | | | |
| 7 | 3291 | ALSSGLYQCVASNAIGTSTC | IGFS11 [207- | 6 | 4614 | QSVPQFFTFEDAPSYPEARA | DMRT1 [302- |
| | | | 226] | | | | 321] |
| 7 | 3292 | NIPPHLQWEVLDSLLVQYGV | KOC1 [87- | 6 | 4615 | QKEITTRYQLDPKFITSILY | EpCAM [167- |
| | | | 106] | | | | 186] |
| 7 | 3293 | PCGARRPDSRLLQLHITMPF | Lage-1 [77- | 6 | 4616 | EFNVLEMEVMRRQLYAVNRR | FATE1 [128- |
| | | | 96] | | | | 147] |
| 7 | 3294 | IPAIVHYSPDCKILVVSNPV | LDHC [121- | 6 | 4617 | KRPSFLDDPLYGISVSLSSE | FSIP1 [533- |
| | | | 140] | | | | 552] |
| 7 | 3295 | YEKKLVQLLVSPPCAPPVME | LEMD1 [34- | 6 | 4618 | KNVNQSLLDLYQLAVEKGDP | FTHL17 [109- |
| | | | 53] | | | | 128] |
| 7 | 3296 | AVITKKVADLVGFLLLKYRA | MAGE- | 6 | 4619 | MSSTDKVIVFVSRKAVADHL | HAGE [484- |
| | | | A1 [100-119] | | | | 503] |
| 7 | 3297 | LGSVIRNFQDFFPVIFSKAS | MAGE- | 6 | 4620 | GDLVDTQSDLIATQRDLIAT | HOM-TES- |
| | | | A12 [136- | | | | 85 [236-255] |
| | | | 155] | | | | |
| 7 | 3298 | GIEVVEVVPISHLYILVTCL | MAGE- | 6 | 4621 | PYSAVEKAMARLQELLTVSE | JARID1B [950- |
| | | | A2 [163-182] | | | | 969] |
| 7 | 3299 | ASNTYTLVTCLGLSYDGLLG | MAGE- | 6 | 4622 | ITMPFSSPMEAELVRRILSR | Lage-1 [92- |
| | | | A4 [173-192] | | | | 111] |
| 7 | 3300 | AGSPGPLKSPQGASAIPTAI | MAGE-A5 [56- | 6 | 4623 | LKGEMMDLQHGSLFFSTSKI | LDHC [58-77] |
| | | | 75] | | | | |
| 7 | 3301 | DPIGHVYIFATCLGLSYDGL | MAGE- | 6 | 4624 | LQGNIILSTEKSKKLKKWPE | LEMD1 [74- |
| | | | A6 [170-189] | | | | 93] |
| 7 | 3302 | KVINYLVMLNAREPICYPSL | MAGE- | 6 | 4625 | EESFSPTAMDAIFGSLSDEG | MAGE-A11 |
| | | | A9 [284-303] | | | | [177-196] |
| 7 | 3303 | PQRAPTTAAAAAAGVSSTKS | MAGE-B2 [62- | 6 | 4626 | PDLESEFQAALSRKVAKLVH | MAGE-A6 [99- |
| | | | 81] | | | | 118] |
| 7 | 3304 | FLGLLGIYDGILHSIYGDAR | MAGE- | 6 | 4627 | VRVNARVRISYPSLHEEALG | MAGE- |
| | | | B6 [310-329] | | | | A8 [294-313] |
| 7 | 3305 | LKRAREFMELLFGLALIEVG | MAGE- | 6 | 4628 | MHFILRKYKMREPIMKADML | MAGE- |
| | | | C2 [182-201] | | | | B1 [116-135] |
| 7 | 3306 | TTAFEAAGIGILTVILGVLL | MART1 [22- | 6 | 4629 | KIVGKRFREHFPEILKKASE | MAGE- |
| | | | 41] | | | | B2 [139-158] |
| 7 | 3307 | LRLDFIHANSTTHSFLFGAL | MORC1 [13- | 6 | 4630 | VQFLMEMYKMKKPIMKADML | MAGE- |
| | | | 32] | | | | B3 [119-138] |
| 7 | 3308 | SEGCVHILDSQTVVLKEPQC | MPHOSPH1 [77- | 6 | 4631 | DEESVSASQKAIIFKRLSKD | MAGE- |
| | | | 96] | | | | B6 [174-193] |
| 7 | 3309 | ELLFRQIAWIKKLPFFCELS | NR6A1 [293- | 6 | 4632 | PRELLTKVWVQGHYLEYREV | MAGE- |
| | | | 312] | | | | C2 [270-289] |
| 7 | 3310 | KHLKHMRRMYGTSLYQFIPL | NYC- | 6 | 4633 | EKPLNSFQYQRRQAMGIPFI | MORC1 [464- |
| | | | TSPG [161- | | | | 483] |
| | | | 180] | | | | |
| 7 | 3311 | LSISSCLQQLSLLMWITQCF | NY-ESO- | 6 | 4634 | KFSVWVSFFEIYNEYIYDLF | MPHOSPH1 [268- |
| | | | 1 [147-166] | | | | 287] |
| 7 | 3312 | ERENRYDCLGSKKYSYMNIC | ODF1 [153- | 6 | 4635 | NGGSSLSYTNPAVAAASANL | MUC-1 [1236- |
| | | | 172] | | | | 1255] |
| 7 | 3313 | KHTPTKLRAPAYSFRGAPML | ODF3 [40-59] | 6 | 4636 | QPERLLFVIDSFEELQGGLN | NLRP4 [222- |
| | | | | | | | 241] |
| 7 | 3314 | TDGVIICVAENISSLLGHLP | PASD1 [50- | 6 | 4637 | QEMVQAFSAQSGMKLEWSQK | NXF2 [573- |
| | | | 69] | | | | 592] |
| 7 | 3315 | SEKKKSVLCSTPTINIPASP | PBK [14-33] | 6 | 4638 | ITKRSEQIVEFLLIKNANAN | NY-BR- |
| | | | | | | | 1 [124-143] |
| 7 | 3316 | YAASIRRTDGGLFLLADVSH | PIWIL2 [363- | 6 | 4639 | ADSVHLAWDLSRSLGAVVFS | OIP5 [88-107] |
| | | | 382] | | | | |
| 7 | 3317 | MKVFKFIGLMILLTSAFSAG | PLAC1 [1-20] | 6 | 4640 | WQQRQFYNKQSTLPRHSTLS | PEPP2 [506- |
| | | | | | | | 525] |
| 7 | 3318 | LSLQCLQALYVDSLFFLRGR | PRAME [293- | 6 | 4641 | ESVGLVSMFRGLGIETVSKT | PIWIL2 [67- |
| | | | 312] | | | | 86] |
| 7 | 3319 | PRSWLLLCPLSHVLFRAILY | PRSS55 [333- | 6 | 4642 | QENPAAFVRAIQQYVDPDVQ | PIWIL4 [526- |
| | | | 352] | | | | 545] |
| 7 | 3320 | NITCCDTDLCNASGAHALQP | PSCA [74-93] | 6 | 4643 | KAVSQDMVIYSTEIHYSSKG | PLAC1 [85- |
| | | | | | | | 104] |
| 7 | 3321 | LSPQCLSLLHAMVAYDPDER | RAGE-1 [254- | 6 | 4644 | DNDIALLLLASPIKLDDLKV | PRSS55 [154- |
| | | | 273] | | | | 173] |
| 7 | 3322 | AAGISSTITRDLYVTATHSV | SAGE1 [657- | 6 | 4645 | EIQALRRLNPHPNILMLHEV | RAGE-1 [50- |
| | | | 676] | | | | 69] |
| 7 | 3323 | TREPAAEIEALLGMDLVRLG | SCRN1 [97- | 6 | 4646 | EEEEDAAWQAEEVLRQQKLA | RCAS1 [160- |
| | | | 116] | | | | 179] |
| 7 | 3324 | IAECTSMIGYALGYVLGAPL | SLCO6A1 [269- | 6 | 4647 | FIQATDYVEIWQAYLDYLRR | SART3 [411- |
| | | | 288] | | | | 430] |
| 7 | 3325 | EIADDLSILYSNRAACYLKE | SPAG1 [481- | 6 | 4648 | MEESNKARAAHSFVVTEFET | SCP-1 [363- |
| | | | 500] | | | | 382] |
| 7 | 3326 | GVWVSIRLDSTLRLYHAHTY | SPAG9 [1101- | 6 | 4649 | RLKTIKQLYEQFIKSMEELE | SCP3a [171- |
| | | | 1120] | | | | 190] |
| 7 | 3327 | INMISGVLQRYCRFGSRPLQ | SSX-3 [151- | 6 | 4650 | GETAKEALDVIVSLLEEHGQ | SCRN1 [120- |
| | | | 170] | | | | 139] |
| 7 | 3328 | KLNYEVMTKLGFKVTLPPFM | SSX-4 [52- | 6 | 4651 | EAGAAALRNVAQRLFENYQT | se57-1 [121- |
| | | | 71] | | | | 140] |
| 7 | 3329 | FLEGCACTPERMAEAGFIHC | Survivin [27- | 6 | 4652 | ISGKEEETSVTILDSSEEDK | SP17 [92-111] |
| | | | 46] | | | | |
| 7 | 3330 | QSGRKLAELQASLSKYCDQL | TDRD1 [515- | 6 | 4653 | IDVVESEAVSVLHHWLKKTE | SPATA19 [28- |
| | | | 534] | | | | 47] |
| 7 | 3331 | ILLVLGASLLTSGLELYCQK | TEX101 [11- | 6 | 4654 | QGIRNLVTDAKFVLIVEKDA | SPO11 [208- |
| | | | 30] | | | | 227] |
| 7 | 3332 | LHFQGFIHRSLSSYAVHIIS | TEX14 [367- | 6 | 4655 | EKRSKAFDDIATYFSKKEWK | SSX-1 [19- |
| | | | 386] | | | | 38] |
| 7 | 3333 | FELYPEFLLVFKRAFHDISH | TMEM31 [91- | 6 | 4656 | GFKAILPSFMRNKRVTDFQG | SSX-3 [62- |
| | | | 110] | | | | 81] |
| 7 | 3334 | LAIALFFLMDVLLRVFVERR | TPTE [131- | 6 | 4657 | ISEKLRKAFDDIAKYFSKKE | SSX-4 [17- |
| | | | 150] | | | | 36] |
| 7 | 3335 | LLKEHLCLAENKMAIQSRDV | TSGA10 [594- | 6 | 4658 | HRVRERKQLVIYEEISDPQE | SSX-5 [166- |
| | | | 613] | | | | 185] |
| 7 | 3336 | FRSNPYFQNKVITKEYLVNI | TSPY1 [205- | 6 | 4659 | QALAPDFRLNPVRRLIPAAR | TAG-1 [413- |
| | | | 224] | | | | 432] |
| 7 | 3337 | EQASRIWSWLLGAAMVGAVL | TYR [469- | 6 | 4660 | IMLLKNFMLNQNVMLSVKGI | TDRD1 [1072- |
| | | | 488] | | | | 1091] |
| 7 | 3338 | ALPVSGAAQWAPVLDFAPPG | WT1 [25-44] | 6 | 4661 | DNIKHSQNMRANSIQLREEA | TEKT5 [301- |
| | | | | | | | 320] |
| 7 | 3339 | EEGGPRSGGAQAKLGCCWGY | XAGE-1c [18- | 6 | 4662 | PKIRDNFWSMPMSEVSQVSR | THEG [238- |
| | | | 37] | | | | 257] |
| 8 | 3340 | LVPDGAVCSNLPYASWFESF | ACRBP [84- | 6 | 4663 | HNALHIYMNGTMSQVQGSAN | TYR [363-382] |
| | | | 103] | | | | |
| 8 | 3341 | YAGGVVLHPRTISLESLAVI | ADAM2 [291- | 6 | 4664 | DLERGTDEAVLQVQAHEHGQ | ZNF165 [126- |
| | | | 310] | | | | 145] |
| 8 | 3342 | MCTPHRSCAIVTFMNKTLGT | ADAM29 [306- | 7 | 4665 | GLGGLRMDSNFDSLPVQITV | ADAM2 [10- |
| | | | 325] | | | | 29] |
| 8 | 3343 | DKLVESVMKLCLIMAKYSND | AKAP-4 [698- | 7 | 4666 | EIKPLAFSTTFEHLVYKMDS | ADAM29 [135- |
| | | | 717] | | | | 154] |
| 8 | 3344 | LEPEDGTALDVHFVSTLEPL | BAGE-2 [31- | 7 | 4667 | ASFVHEYSRRHPQLAVSVIL | AFP [352-371] |
| | | | 50]; BAGE- | | | | |
| | | | 3 [31-50] | | | | |
| 8 | 3345 | FAADVRLMFMNCYKYNPPDH | BRDT [337- | 7 | 4668 | DDFTASVSEGIMTYANSVVS | AKAP-3 [275- |
| | | | 356] | | | | 294] |
| 8 | 3346 | SGIINFIHFREACRETYMEF | CDCA1 [122- | 7 | 4669 | SDTSGDFRKLMVALAKGRRA | ANXA2 [161- |
| | | | 141] | | | | 180] |
| 8 | 3347 | RCGENLYMSSDPTSWSSAIQ | CRISP2 [93- | 7 | 4670 | VDAVKLQLPDYYTIIKNPMD | BRDT [56-75] |
| | | | 112] | | | | |
| 8 | 3348 | NESSMLSTDTKKASILLIRK | CT46 [137- | 7 | 4671 | TMYRGNTTMDIKDLVKQFHQ | CABYR [45- |
| | | | 156] | | | | 64] |
| 8 | 3349 | ANFDLAVVARRYPASGIHFV | CT47 [103- | 7 | 4672 | SRQMVTDLELSTLLPISHEN | CCDC62 [641- |
| | | | 122] | | | | 660] |
| 8 | 3350 | YIDQFLLTSFPTFTSVGVLI | CTAGE1 [35- | 7 | 4673 | TLSFPRYNVAEIVIHIRNKI | CDCA1 [3-22] |
| | | | 54] | | | | |
| 8 | 3351 | FAMRNVYLPRGFLPYRPPRP | CTAGE2 [717- | 7 | 4674 | AMSKAKKLMTGHAIPPSQLD | CT45 [51-70] |
| | | | 736] | | | | |
| 8 | 3352 | PKYQCPHCATIIARKSDLRV | CTCFL [426- | 7 | 4675 | DALQKKYLRMVVLAVYTNPE | CT46 [87-106] |
| | | | 445] | | | | |
| 8 | 3353 | VVLTVKASYVASTKYQKVYG | CTNNA2 [876- | 7 | 4676 | LTERLLKMKDGVAMLEEDVT | CTAGE2 [237- |
| | | | 895] | | | | 256] |
| 8 | 3354 | PQVDDIVNVVMVESIQFCFI | CXorf48 [211- | 7 | 4677 | SKSLKSQVAEAKMTFKIFQM | CTAGE5 [180- |
| | | | 230] | | | | 199] |
| 8 | 3355 | LGTVKWFNVRNGYGFINRND | DBPC [95- | 7 | 4678 | KSDLRVHMRNLHAYSAAELK | CTCFL [440- |
| | | | 114] | | | | 459] |
| 8 | 3356 | SGIRSVSFYEHIITVGTGQG | DCAF12 [341- | 7 | 4679 | FGKEMVKLNYVAARRQQELK | CTNNA2 [179- |
| | | | 360] | | | | 198] |
| 8 | 3357 | KNMENRHAMSSQYRMHSYYP | DMRT1 [276- | 7 | 4680 | KRILVNLSMVENKLVELEHT | CXorf61 [78- |
| | | | 295] | | | | 97] |
| 8 | 3358 | TKGWVRLQFHAGQAWVPTTH | DPPA2 [235- | 7 | 4681 | NRRKSRRFIPRPPSVAPPPM | DBPC [176- |
| | | | 254] | | | | 195] |
| 8 | 3359 | LAAATATFAAAQEECVCENY | EpCAM [13- | 7 | 4682 | LQGFSRLFLKGNLLRGIDSL | DKKL1 [52- |
| | | | 32] | | | | 71] |
| 8 | 3360 | VSGLVTSRSFRTASVSINQT | EPHA2 [418- | 7 | 4683 | ASEGRMVIQDIPAVTSRGHV | DMRT1 [178- |
| | | | 437] | | | | 197] |
| 8 | 3361 | EPPPVSFQPYHPLHFRGSNG | FAM46D [368- | 7 | 4684 | PGHQKRIAYSLLGLKDQVNT | EPHA2 [952- |
| | | | 387] | | | | 971] |
| 8 | 3362 | GQSLEEDSALEALLNFFFPT | FMR1NB [113- | 7 | 4685 | LDPMLDFYSDKNAKLTKESY | FAM46D [180- |
| | | | 132] | | | | 199] |
| 8 | 3363 | CILKQKSIIKLSSERKKEDI | FSIP1 [415- | 7 | 4686 | PGTDAVAQTSLEEFNVLEME | FATE1 [116- |
| | | | 434] | | | | 135] |
| 8 | 3364 | GWTPLMYAASVANAELVRVL | GASZ [79-98] | 7 | 4687 | AIQKMKKLDKILAKKQRREK | FSIP1 [118- |
| | | | | | | | 137] |
| 8 | 3365 | DCVLYRYGSFSVTLDIVQGI | gp100 [474- | 7 | 4688 | HFLESHYLHEQVKTIKELGG | FTHL17 [132- |
| | | | 493] | | | | 151] |
| 8 | 3366 | HEELPLCFALKSHFVGAVIG | HAGE [65-84] | 7 | 4689 | EKIKQRLFENLRMLPHAPGV | HDAC2 [361- |
| | | | | | | | 380]; |
| | | | | | | | HDAC1 [360- |
| | | | | | | | 379] |
| 8 | 3367 | GILELLKYHPRVLYIDIDIH | HDAC3 [153- | 7 | 4690 | TIFENLKMLNHAPSVQIHDV | HDAC3 [361- |
| | | | 172] | | | | 380] |
| 8 | 3368 | VGFTGTMPATNVSIFINNTQ | IGFS11 [92- | 7 | 4691 | PSQKPSGFKSGQHPLNGQPL | HOM-TES- |
| | | | 111] | | | | 85 [92-111] |
| 8 | 3369 | LPPVYLTFTRESSCEIKLKW | IL13RA2 [239- | 7 | 4692 | GSETWKTIITKNLHYKDGFD | IL13RA2 [77- |
| | | | 258] | | | | 96] |
| 8 | 3370 | VDILTYIVWKISGLPVTRVI | LDHC [140- | 7 | 4693 | IEEIPAYLPNGAALKDSVQR | JARID1B [997- |
| | | | 159] | | | | 1016] |
| 8 | 3371 | DHFPLLFSEASECMLLVFGI | MAGE- | 7 | 4694 | DVGYGRSISSSSSLRRSSKE | KU-CT- |
| | | | A10 [170- | | | | 1 [621-640] |
| | | | 189] | | | | |
| 8 | 3372 | EYLQLVFGIEVVEVVRIGHL | MAGE- | 7 | 4695 | SRGATTFIYNRAVKNTRKVA | LIPI [135- |
| | | | A12 [156- | | | | 154] |
| | | | 175] | | | | |
| 8 | 3373 | GPRALIETSYVKVLHHTLKI | MAGE- | 7 | 4696 | PRAHAEIRKMSLLKFLAKVN | MAGE-A10 [300-319] |
| | | | A2 [274-293] | | | | |
| 8 | 3374 | SSSSPLVPGTLGEVPAAGSP | MAGE-A5 [40- | 7 | 4697 | VKGLITKAEMLGSVIKNYED | MAGE-A11 [239-258] |
| | | | 59] | | | | |
| 8 | 3375 | RALIETSYVKVLHHMVKISG | MAGE- | 7 | 4698 | EEQEAASSSSTLVEVTLGEV | MAGE-A3 [34- |
| | | | A6 [276-295] | | | | 53]; MAGE- |
| | | | | | | | A6 [34-53] |
| 8 | 3376 | AGHSYILVTCLGLSYDGLLG | MAGE- | 7 | 4699 | EERAQVRSSVRARRRTTATT | MAGE- |
| | | | A8 [175-194] | | | | B1 [312-331] |
| 8 | 3377 | WGPRAYAETTKMKVLEFLAK | MAGE- | 7 | 4700 | IEKKQSFSQGLSSTVQSRTD | MAGE-B3 [90- |
| | | | B1 [272-291] | | | | 109] |
| 8 | 3378 | TDEESLLSSWDFPRRKLLMP | MAGE- | 7 | 4701 | FPEIFRKVSQRTELVFGLAL | MAGE- |
| | | | B2 [187-206] | | | | B4 [147-166] |
| 8 | 3379 | SDPPCYEFLWGPRAYAETTK | MAGE- | 7 | 4702 | SLLQIPMTSSFSSTLLSIFQ | MAGE- |
| | | | B6 [350-369] | | | | C1 [336-355] |
| 8 | 3380 | IGILTVILGVLLLIGCWYCR | MART1 [30- | 7 | 4703 | TSPQLSTGVSFFFLSFHISN | MUC-1 [1032- |
| | | | 49] | | | | 1051] |
| 8 | 3381 | EESLSEVVVPMPSWLIRTRE | MORC1 [136- | 7 | 4704 | TETQVKIWFQNRRYKTKRKQ | NKX3.1 [164- |
| | | | 155] | | | | 183] |
| 8 | 3382 | NISQCYLAYDETLNVLKFSA | MPHOSPH1 [455- | 7 | 4705 | MQDPAFVKQAVNLLQEANFH | NLRP4 [560- |
| | | | 474] | | | | 579] |
| 8 | 3383 | SSLRKLCFSVQNVFKKEDEH | NLRP4 [597- | 7 | 4706 | QMEMLKLTMNKRYNVSQQAL | NXF2 [213- |
| | | | 616] | | | | 232] |
| 8 | 3384 | QDNEWNYTRAGQAFTMLQTE | NXF2 [595- | 7 | 4707 | AQRKSKSLKINLNYAGDALR | NY-BR- |
| | | | 614] | | | | 1 [1200- |
| | | | | | | | 1219] |
| 8 | 3385 | HEEVVTFLVDRKCQLDVLDG | NY-BR-1 [29- | 7 | 4708 | ATSAQNIEFLQVIAKRERAI | ODF2 [675- |
| | | | 48] | | | | 694] |
| 8 | 3386 | CGARGPESRLLEFYLAMPFA | NY-ESO- | 7 | 4709 | PINIWIFELERNVSIPIGWS | ODF4 [163- |
| | | | 1 [78-97] | | | | 182] |
| 8 | 3387 | LASSCCSSNILGSVNVCGFE | ODF1 [116- | 7 | 4710 | THNRLKSLMKILSEVTPDQS | OIP5 [206- |
| | | | 135] | | | | 225] |
| 8 | 3388 | DKMVCYLLKTKAIVNASEMD | OIP5 [167- | 7 | 4711 | SQEIEMHADNPAAIQTVVLQ | PEPP2 [674- |
| | | | 186] | | | | 693] |
| 8 | 3389 | PEVNPLYRADPVDLEFSVDQ | PASD1 [291- | 7 | 4712 | YNPLMEARRLRSALLFQHED | PIWIL1 [136- |
| | | | 310] | | | | 155] |
| 8 | 3390 | DKADIFAFGLTLWEMMTLSI | PBK [226- | 7 | 4713 | FGERHIFDGNSLLLSRPLKE | PIWIL3 [170- |
| | | | 245] | | | | 189] |
| 8 | 3391 | GLKPDHMQRLTFKLCHLYYN | PIWIL4 [793- | 7 | 4714 | SNKAKAFDGAILFLSQKLEE | PIWIL4 [151- |
| | | | 812] | | | | 170] |
| 8 | 3392 | VTECGIRAKAVSQDMVIYST | PLAC1 [77- | 7 | 4715 | LKKEGWRPRRTILFASWDAE | PSMA [405- |
| | | | 96] | | | | 424] |
| 8 | 3393 | EVGEFPWQVSIQARSEPFCG | PRSS55 [75- | 7 | 4716 | QQRKKMEKEAQRLMKKEQNK | RCAS1 [188- |
| | | | 94] | | | | 207] |
| 8 | 3394 | LHEVVFDRKSGSLALICELM | RAGE-1 [66- | 7 | 4717 | EKVHSLFRRQLAIPLYDMEA | SART3 [245- |
| | | | 85] | | | | 264] |
| 8 | 3395 | VFSFLKRLICRSGRGRKLSG | RCAS1 [18- | 7 | 4718 | EQEQSSLRASLEIELSNLKA | SCP-1 [739- |
| | | | 37] | | | | 758] |
| 8 | 3396 | SLEKHKLFISGLPFSCTKEE | SART3 [797- | 7 | 4719 | NLLTGAQNEFKKEMAMLQKK | SCP3a [195- |
| | | | 816] | | | | 214] |
| 8 | 3397 | AEGSKAMNGSAAKLQQYYCW | SOX-6 [576- | 7 | 4720 | ERKLSLENKLLQLKSSATYG | se57-1 [207- |
| | | | 595] | | | | 226] |
| 8 | 3398 | MTFGRLHRIIPKIMPKKPAE | SSX-1 [99- | 7 | 4721 | SYNHKQIEQLYAAQLASMQV | SOX-6 [360- |
| | | | 118] | | | | 379] |
| 8 | 3399 | TENEPDLAQCFFCFKELEGW | Survivin [48- | 7 | 4722 | REILREQPDNIPAFAAAYFE | SP17 [25-44] |
| | | | 67] | | | | |
| 8 | 3400 | ACTVRNLARSQSVKMKDKLK | TAF7L [111- | 7 | 4723 | HLSKSDLLANQSQEVLEERT | SPATA19 [93- |
| | | | 130] | | | | 112] |
| 8 | 3401 | SIKARFPAETYALVGQQVTL | TAG-1 [240- | 7 | 4724 | ASIENIIQDIITSLARNEAP | SPO11 [49- |
| | | | 259] | | | | 68] |
| 8 | 3402 | PLVSRDPPASASLFQDTCAG | TAG-2a [67- | 7 | 4725 | GKHAWTHRLRERKQLVIYEE | SSX-1 [160- |
| | | | 86] | | | | 179] |
| 8 | 3403 | AVGPMFVREACPHQLLTQPR | TEX101 [199- | 7 | 4726 | QIPEKIQKAFDDIAKYFSKE | SSX-2 [16- |
| | | | 218] | | | | 35] |
| 8 | 3404 | VGPPSLNYIPPVLQLSGGQK | TEX14 [794- | 7 | 4727 | KYFSEKEWEKMKASEKIIYV | SSX-5 [30- |
| | | | 813] | | | | 49] |
| 8 | 3405 | KAQMEKIGLPIILHLFALST | TMEM31 [113- | 7 | 4728 | AEGPSTLDEGLFLRSQEAAA | SYCE1 [219- |
| | | | 132] | | | | 238] |
| 8 | 3406 | YFWLHTSFIENNRLYLPKNE | TPTE [495- | 7 | 4729 | DADSSAQAAAQAPENFQEGK | TAF7L [66- |
| | | | 514] | | | | 85] |
| 8 | 3407 | EAPPIYLQVSSYQHWIWDCL | TSP50 [338- | 7 | 4730 | VSREAILRFGFLQEFSKEER | TAG-1 [121- |
| | | | 357] | | | | 140] |
| 8 | 3408 | TLAKHTISSDYVIPIGTYGQ | TYR [139- | 7 | 4731 | GGAESERGLPASTLSRLSNR | TAG-2a [29- |
| | | | 158] | | | | 48] |
| 8 | 3409 | WAPVLDFAPPGASAYGSLGG | WT1 [34-53] | 7 | 4732 | GFWKSELSYELDRLLTENQN | TEKT5 [149- |
| | | | | | | | 168] |
| 8 | 3410 | GGAQAKLGCCWGYPSPRSTW | XAGE-1c [25- | 7 | 4733 | ALKSRISWEGLLALDNGEME | TEX15 [712- |
| | | | 44] | | | | 731] |
| 8 | 3411 | LKQELCRQLFRQFCYQDSPG | ZNF165 [43- | 7 | 4734 | RPKRFYLEYYNNNRTTPVWP | THEG [199- |
| | | | 62] | | | | 218] |
| 9 | 3412 | PAGAFARRPPLAELAALNLS | 5T4 [107- | 7 | 4735 | VLGEAWRDQVDWSRLLRDAG | TRAG-3 [45- |
| | | | 126] | | | | 64] |
| 9 | 3413 | FKSQQCLMRNRNRKVSRMRC | ACRBP [490- | 7 | 4736 | SDVPVLQVIMHSRYRAQRFW | TSP50 [179- |
| | | | 509] | | | | 198] |
| 9 | 3414 | SVESCEISLRPLLVSHVMAC | ACTL8 [279- | 7 | 4737 | FKWNSDFINHQIIYAGEKNH | ZNF165 [301- |
| | | | 298] | | | | 320] |
| 9 | 3415 | EELQKYIQESQALAKRSCGL | AFP [399- | 8 | 4738 | LRHLDLSNNSLVSLTYVSFR | 5T4 [236-255] |
| | | | 418] | | | | |
| 9 | 3416 | AVSPEFAYVPADPAQLAAQM | CABYR [160- | 8 | 4739 | KTLEFAGQDLSAYLLKSLFK | ACTL8 [171- |
| | | | 179] | | | | 190] |
| 9 | 3417 | ECADQRLAISHSQIAHLEER | CAGE1 [713- | 8 | 4740 | YKEVSKMVKDALTAIEKPTG | AFP [60-79] |
| | | | 732] | | | | |
| 9 | 3418 | REKLKSQEIFLNLKTALEKY | CDCA1 [414- | 8 | 4741 | LMTDTQFVSAVKRTVFSHGS | AKAP-3 [348- |
| | | | 433] | | | | 367] |
| 9 | 3419 | QLVWYSTYQVGCGIAYCPNQ | CRISP2 [139- | 8 | 4742 | PKWISIMTERSVPHLQKVFD | ANXA2 [211- |
| | | | 158] | | | | 230] |
| 9 | 3420 | KRKLVKELRCVGQKYEKIFE | CT45 [127- | 8 | 4743 | EVPAQLLDAEGAIKIGSEKS | CABYR [397- |
| | | | 146] | | | | 416] |
| 9 | 3421 | NKISTEHQSLVLVKRLLAVS | CT46 [18-37] | 8 | 4744 | EKVSDIMLQKLKSLHLKKKT | CAGE1 [645- |
| | | | | | | | 664] |
| 9 | 3422 | YLPRGFLPYRPPRPAFFPPA | CTAGE2 [723- | 8 | 4745 | DSRFGHFRLSSGKFYGHKEK | CALR3 [43- |
| | | | 742] | | | | 62] |
| 9 | 3423 | LPQGDTQLTTVQGVVTSFCG | CXorf48 [29- | 8 | 4746 | VEWMSIFKPSKMQRIVRLKS | CCDC62 [532- |
| | | | 48] | | | | 551] |
| 9 | 3424 | PPARSQADKPVLAIQVLGTV | DBPC [79-98] | 8 | 4747 | SQIDDFTGFSKDRMMQKPGS | CT45 [71-90] |
| 9 | 3425 | AENTLSKLLSTKLPYCRENV | DCAF12 [281- | 8 | 4748 | LLEKDPYALDVPNTAFGREH | CTAGE5 [497- |
| | | | 300] | | | | 516] |
| 9 | 3426 | PYYNNLYNCPQYSMALAADS | DMRT1 [220- | 8 | 4749 | NSTALALVRPSSSGLINSNT | CXorf61 [34- |
| | | | 239] | | | | 53] |
| 9 | 3427 | KALNSCSIPVSVEAFLMQAS | DPPA2 [200- | 8 | 4750 | PRMEEKEALVPIQKATDSFH | DKKL1 [141- |
| | | | 219] | | | | 160] |
| 9 | 3428 | ALLSVRVYYKKCPELLQGLA | EPHA2 [190- | 8 | 4751 | TRPKKMGSQLPKPRMLRESG | FATE1 [73- |
| | | | 209] | | | | 92] |
| 9 | 3429 | CQILDSLSYMRNENVISELN | FBXO39 [180- | 8 | 4752 | MGKRLDYLNLKGARLTVEQG | FBXO39 [160- |
| | | | 199] | | | | 179] |
| 9 | 3430 | CYYLSYYLCSGSSYFVLANG | FMR1NB [83- | 8 | 4753 | LSDDKMEHAQKLMRLQNLRG | FTHL17 [59- |
| | | | 102] | | | | 78] |
| 9 | 3431 | KYDTNCDAAINSHITLELYT | FTHL17 [12- | 8 | 4754 | KLESSHSRGSMTALGGASSS | GATA-3 [195- |
| | | | 31] | | | | 214] |
| 9 | 3432 | NGYTALTWAARQGHKNIVLK | GASZ [181- | 8 | 4755 | YYPEDLFIDKKVLKVAHVEH | Glypican- |
| | | | 200] | | | | 3 [361-380] |
| 9 | 3433 | HVPPYYGNSVRATVQRYPPT | GATA-3 [59- | 8 | 4756 | NHHSERSRNHLERSLSQSDR | HOM-TES- |
| | | | 78] | | | | 85 [156-175] |
| 9 | 3434 | RCLLHLAVIGALLAVGATKV | gp100 [7-26] | 8 | 4757 | EGDALRYMIERTVNWQHRAQ | JARID1B [1248- |
| | | | | | | | 1267] |
| 9 | 3435 | KCVEVVKTFNLPLLMLGGGG | HDAC2 [284- | 8 | 4758 | LDKLNGFQLENFTLKVAYIP | KOC1 [136- |
| | | | 303] | | | | 155] |
| 9 | 3436 | INQVVDFYQPTCIVLQCGAD | HDAC3 [240- | 8 | 4759 | LLRELDVMNSVIAQLAPEEE | KU-CT- |
| | | | 259] | | | | 1 [103-122] |
| 9 | 3437 | SKKTLLRFWLPFGFILILVI | IL13RA2 [337- | 8 | 4760 | REGAGRMRVVGWGLGSASPE | Lage-1 [142- |
| | | | 356] | | | | 161] |
| 9 | 3438 | DLLHQLVTIMNPNTLMTHEV | JARID1B [547- | 8 | 4761 | LSTEKSKKLKKWPEASTTKR | LEMD1 [80- |
| | | | 566] | | | | 99] |
| 9 | 3439 | EAHIRVPSFAAGRVIGKGGK | KOC1 [489- | 8 | 4762 | GSIPLWLQNFVRILLNEEDM | LIPI [108- |
| | | | 508] | | | | 127] |
| 9 | 3440 | IIKLKGYTSWAIGLSVMDLV | LDHC [241- | 8 | 4763 | QSPQGASAFPTTINFTRQRQ | MAGE-A1 [56- |
| | | | 260] | | | | 75] |
| 9 | 3441 | TTKRKAVDTYCLDYKPSKGR | LEMD1 [96- | 8 | 4764 | GSVIKNYEDYFPEIFREASV | MAGE-A11 [250-269] |
| | | | 115] | | | | |
| 9 | 3442 | FIKCNHQRAVHLFMASLETN | LIPI [269- | 8 | 4765 | KLLTQHFVQENYLEYRQVPG | MAGE- |
| | | | 88] | | | | A3 [244-263] |
| 9 | 3443 | EEALEAQQEALGLVCVQAAT | MAGE-A1 [13- | 8 | 4766 | TTEEQEAAVSSSSPLVPGTL | MAGE-A4 [32- |
| | | | 32] | | | | 51] |
| 9 | 3444 | GPITQIFPTVRPADLTRVIM | MAGE- | 8 | 4767 | VGKEHMFYGEPRKLLTQDWV | MAGE- |
| | | | A11 [92-111] | | | | A9 [231-250] |
| 9 | 3445 | ALVETSYVKVLHHLLKISGG | MAGE- | 8 | 4768 | FWAKVNKTVPSAFQFWYEEA | MAGE- |
| | | | A12 [277- | | | | B3 [291-310] |
| | | | 296] | | | | |
| 9 | 3446 | EYLQLVFGIEVVEVVPISHL | MAGE- | 8 | 4769 | EPTTKAEMLKIISKKYKEHF | MAGE- |
| | | | 2 [156-175] | | | | B4 [128-147] |
| 9 | 3447 | LERVIKNYKRCFPVIFGKAS | MAGE- | 8 | 4770 | IESEPLFTYTLDEKVDELAR | MAGE- |
| | | | A4 [137-156] | | | | C1 [898-917] |
| 9 | 3448 | APEEVIWEALSVMGVYVGKE | MAGE- | 8 | 4771 | KKKVLEFLAKLNNTVPSSFP | MAGE- |
| | | | A9 [215-234] | | | | C2 [311-330] |
| 9 | 3449 | PITKAEMLTNVISRYTGYFP | MAGE- | 8 | 4772 | AASRYNLTISDVSVSDVPFP | MUC-1 [1128- |
| | | | C1 [928-947] | | | | 1147] |
| 9 | 3450 | MPREDAHFIYGYPKKGHGHS | MART1 [1-20] | 8 | 4773 | PTPSKPLTSFLIQDILRDGA | NKX3.1 [20- |
| | | | | | | | 39] |
| 9 | 3451 | IEDSEMSRVIRVSELSLCDL | MPHOSPH1 [362- | 8 | 4774 | SSRSVELNGFMAFREQYMGM | NR6A1 [198- |
| | | | 381] | | | | 217] |
| 9 | 3452 | CFPKLLRLDGRELSAPVIVD | NXF2 [349- | 8 | 4775 | SNLQNNYAHLTNSSINKSGA | NYC- |
| | | | 368] | | | | TSPG [288- |
| | | | | | | | 307] |
| 9 | 3453 | SEIVGMLLQQNVDVFAADIC | NY-BR- | 8 | 4776 | KLVEAEMDGAAAAKQVMALK | ODF2 [210- |
| | | | 1 [162-181] | | | | 229 |
| 9 | 3454 | CIDEFSTRCLCDLYMHPYCC | ODF1 [27-46] | 8 | 4777 | SAPSHSISARTKAFRVDSTP | ODF3 [118- |
| | | | | | | | 137] |
| 9 | 3455 | IWIFELERNVSIPIGWSYFI | ODF4 [166- | 8 | 4778 | AFSKKWLDLSRSLFYQRWPV | ODF4 [99-118] |
| | | | 185] | | | | |
| 9 | 3456 | ERCAVFQCAQCHAVLADSVH | OIP5 [73-92] | 8 | 4779 | DLNLEWISLPRSWTYGITRG | PEPP2 [4-23] |
| 9 | 3457 | AVYVEPAAAAAAAAISDDQI | PASD1 [223- | 8 | 4780 | NSLIQNLFKVTPAMGMQMRK | PIWIL1 [509- |
| | | | 242] | | | | 528] |
| 9 | 3458 | DFDDEAYYAALGTRPPINME | PBK [265- | 8 | 4781 | KWYSRVVFQMPHQEIVDSLK | PIWIL2 [770- |
| | | | 284] | | | | 789] |
| 9 | 3459 | KKAIQLYRHGTSLEIWLGYV | PIWIL3 [249- | 8 | 4782 | LHSWLILYSRSSHREAMSLK | PIWIL3 [507- |
| | | | 268] | | | | 526] |
| 9 | 3460 | TRNEWYDFYLISQVACRGTV | PIWIL4 [761- | 8 | 4783 | QLTTLSFYGNSISISALQSL | PRAME [407- |
| | | | 780] | | | | 426] |
| 9 | 3461 | LLTSAFSAGSGQSPMTVLCS | PLAC1 [12- | 8 | 4784 | EMKTYSVSFDSLFSAVKNFT | PSMA [621- |
| | | | 31] | | | | 640] |
| 9 | 3462 | LLKDEALAIAALELLPRELF | PRAME [34- | 8 | 4785 | PAPDNVLLTLRPRRINMTDT | SAGE1 [216- |
| | | | 53] | | | | 235] |
| 9 | 3463 | ECGDRSIFEGRTRYSRITGG | PRSS55 [52- | 8 | 4786 | KDKRDYLWTSAKNTLSTPLP | SCP-1 [833- |
| | | | 71] | | | | 852] |
| 9 | 3464 | YPANEYAYRRGIAEAVGLPS | PSMA [272- | 8 | 4787 | QEEKILNMFRQQQKILQQSR | SCP3a [146- |
| | | | 291] | | | | 165] |
| 9 | 3465 | QPYTEYISTRWYRAPECLLT | RAGE-1 [156- | 8 | 4788 | DQEQGRKLRSTMLELEKQGL | SCRN1 [361- |
| | | | 175] | | | | 380] |
| 9 | 3466 | PPQLVHMAAAGIPSMSTRDL | SAGE1 [132- | 8 | 4789 | KKIRTTLQKIRTQMFKDEIR | se57-1 [38- |
| | | | 151] | | | | 57] |
| 9 | 3467 | RRKVLSRAVAAATYKTMGPA | SART3 [35- | 8 | 4790 | QSSGISFQSKYLSFFILGQT | SLCO6A1 [218- |
| | | | 54] | | | | 237] |
| 9 | 3468 | AAPPSYCFVAFPPRAKDGLV | SCRN1 [3-22] | 8 | 4791 | TPNNHFTLEDIQALKRQYEL | SPAG1 [907- |
| | | | | | | | 926] |
| 9 | 3469 | LCTIIFTTIAFFIYKRRLNE | SLCO6A1 [678- | 8 | 4792 | GYRNKIYVVQPKAMKIEKSF | SPAG9 [1063- |
| | | | 697] | | | | 1082] |
| 9 | 3470 | YAAQLASMQVSPGAKMPSTP | SOX-6 [370- | 8 | 4793 | EMTEDIMRDRIEQVRRSISR | SPATA19 [130- |
| | | | 389] | | | | 149] |
| 9 | 3471 | CEKHLQALAPESRALRKDKP | SPAG1 [61- | 8 | 4794 | YATKRDIYYTDSQLFGNQTV | SPO11 [130- |
| | | | 80] | | | | 149] |
| 9 | 3472 | TFIPYCSMAHAQLCFHGHRD | SPAG9 [1205- | 8 | 4795 | MNGDDAFARRPTVGAQIPEK | SSX-2 [1-20] |
| | | | 1224] | | | | |
| 9 | 3473 | PPGKPTTSEKINMISGVLQR | SSX-3 [141- | 8 | 4796 | MNGDDTFARRPTVGAQIPEK | SSX-3 [1-20] |
| | | | 160] | | | | |
| 9 | 3474 | FHMPERLAKEICALDSSKEQ | SYCE1 [175- | 8 | 4797 | VTLPPFMRSKRAADFHGNDF | SSX-4 [65- |
| | | | 194] | | | | 84] |
| 9 | 3475 | TLPLCRVQPITSSHLALPFQ | TDRD1 [1032- | 8 | 4798 | RGKHAWTHRVRERKQLVIYE | SSX-5 [159- |
| | | | 1051] | | | | 178] |
| 9 | 3476 | SSPPGLIVTSYSNYCEDSFC | TEX101 [95- | 8 | 4799 | VHLKEILSKKQETLRILRLH | SYCE1 [96- |
| | | | 114] | | | | 115] |
| 9 | 3477 | CKMSLHFCLCWPSVYWTERF | THEG [158- | 8 | 4800 | LGVTKEIAIWAERIMFSDLR | TDRD1 [237- |
| | | | 177] | | | | 256] |
| 9 | 3478 | FLLVFKEAFHDISHCLKAQM | TMEM31 [97- | 8 | 4801 | LVNEVFTIDDTLQTLKLRLR | TEKT5 [415- |
| | | | 116] | | | | 434] |
| 9 | 3479 | DRTGTMVCAFLIASEICSTA | TPTE [343- | 8 | 4802 | YGLEHIFFDAAKNLVWKERT | TEX15 [1882- |
| | | | 362] | | | | 1901] |
| 9 | 3480 | HCLIWRDVIYSVRVGSPWID | TSP50 [153- | 8 | 4803 | PSTTMTKARKRRRRRRLMEL | THEG [112- |
| | | | 172] | | | | 131] |
| 9 | 3481 | DSCTGSQALLLRTPYSSDNL | WT1 [207- | 8 | 4804 | KARQSEADNNTLKLELITAE | TSGA10 [430- |
| | | | 226] | | | | 449] |
| 10 | 3482 | LASNHFLYLPRDVLAQLPSL | 5T4 [217- | 8 | 4805 | HHNSSLNFFNWFSDHNFAGS | TSPY1 [250- |
| | | | 236] | | | | 269] |
| 10 | 3483 | RKPAAGFLPSLLKVLLLPLA | ACRBP [2-21] | 9 | 4806 | QDFAKYIEMHVIVEKQLYNH | ADAM2 [174- |
| | | | | | | | 193] |
| 10 | 3484 | ENPGPSYARRRVSLGIDICH | ACTL8 [40- | 9 | 4807 | DSEEKQFSTMRSGFMQNEIT | ADAM29 [153- |
| | | | 59] | | | | 172] |
| 10 | 3485 | LTNAIFVSFNITIILSSLEL | ADAM2 [211- | 9 | 4808 | KQLQAVLQWVAASELNVPIL | AKAP-3 [771- |
| | | | 230] | | | | 790] |
| 10 | 3486 | ILQINDFAYEIKPLAFSTTF | ADAM29 [126- | 9 | 4809 | MTDSDFVSAVKRNLFNQWKQ | AKAP-4 [398- |
| | | | 145] | | | | 417] |
| 10 | 3487 | ENCYSVYADQVNIDYLMNRP | AKAP-4 [153- | 9 | 4810 | RYKSYSPYDMLESIRKEVKG | ANXA2 [231- |
| | | | 172] | | | | 250] |
| 10 | 3488 | VSNLSQGVMLSHSPICMETT | CAGE1 [38- | 9 | 4811 | LKAVHQQLQVLSQVPFRKLN | BRDT [430- |
| | | | 57] | | | | 449] |
| 10 | 3489 | AIGLELWQVRSGTIFDNFLI | CALR3 [299- | 9 | 4812 | AISARFKPFSNKGKTLVIQY | CALR3 [76- |
| | | | 318] | | | | 95] |
| 10 | 3490 | TKKASILLIRKIYILMQNLG | CT46 [146- | 9 | 4813 | LHNLRQIYVKQQSDLQFLNF | CCDC62 [282- |
| | | | 165] | | | | 301] |
| 10 | 3491 | QLSRLMVGPHAAARNLWGNL | CT47 [146- | 9 | 4814 | EEGEQAAGLAAVPRGGSAEE | CT47 [64-83] |
| | | | 165] | | | | |
| 10 | 3492 | ELYQENEMKLYRKLIVEEKC | CTAGE2 [357- | 9 | 4815 | LSGPAELRSFNMPSLDKMDG | cTAGE5 [634- |
| | | | 376] | | | | 653] |
| 10 | 3493 | PPPAPFAMRNVYPPRGFPPY | CTAGE5 [743- | 9 | 4816 | MSGDERSDEIVLTVSNSNVE | CTCFL [206- |
| | | | 762] | | | | 225] |
| 10 | 3494 | ACKQDLLAYLQRIALYCHQL | CTNNA2 [770- | 9 | 4817 | LVYDGVRDIRKAVLMIRTPE | CTNNA2 [615- |
| | | | 789] | | | | 634] |
| 10 | 3495 | GPSDSGTRVLIGCVTSINED | CXorf48 [105- | 9 | 4818 | LEHTLLSKGFRGASPHRKST | CXorf61 [94- |
| | | | 124] | | | | 113] |
| 10 | 3496 | SGQRPRRWCPPPFFYRRRFV | DBPC [216- | 9 | 4819 | VAFWIIKLPRRRSHQDALEG | DKKL1 [167- |
| | | | 235] | | | | 186] |
| 10 | 3497 | DSSGTKLFVAGGPLPSGLHG | DCAF12 [427- | 9 | 4820 | KTQNDVDIADVAYYFEKDVK | EpCAM [202- |
| | | | 446] | | | | 221] |
| 10 | 3498 | YPPPSYLGQSVPQFFTFEDA | DMRT1 [294- | 9 | 4821 | IVKDARLNGSVASYILASHN | FAM46D [57- |
| | | | 313] | | | | 76] |
| 10 | 3499 | LASWARIAARAVQPKALNSC | DPPA2 [186- | 9 | 4822 | LQEYAGNFQGIRFHYDRNPG | FATE1 [98- |
| | | | 205] | | | | 117] |
| 10 | 3500 | EKAPEFSMQGLKAGVIAVIV | EpCAM [254- | 9 | 4823 | EMENTKKFLSLTAVSEETVG | FSIP1 [168- |
| | | | 273] | | | | 187] |
| 10 | 3501 | SCKETFNLYYAESDLDYGTN | EPHA2 [114- | 9 | 4824 | VLLGLFSTIHDSIQYVQKNA | Glypican- |
| | | | 133] | | | | 3 [319-338] |
| 10 | 3502 | LRHKCCFSSSGTTSFKCFAP | FMR1NB [157- | 9 | 4825 | PKASTWVVASRRSSTVSRAP | HAGE [8-27] |
| | | | 176] | | | | |
| 10 | 3503 | AQKLMRLQNLRGGHICLHDI | FTHL17 [67- | 9 | 4826 | NQPEQVILYQGGQMFDGAPR | IGFS11 [68- |
| | | | 86] | | | | 87] |
| 10 | 3504 | TTYPPYVPEYSSGLFPPSSL | GATA-3 [220- | 9 | 4827 | KKNSYHFSAGFGSPIEDKSE | KU-CT- |
| | | | 239] | | | | 1 [643-662] |
| 10 | 3505 | DDMVNELFDSLFPVIYTQLM | Glypican- | 9 | 4828 | VLKDFTVSGNLLFMSVRDQD | Lage-1 [122- |
| | | | 3 [164-183] | | | | 141] |
| 10 | 3506 | CLNVSLADTNSLAVVSTQLI | gp100 [566- | 9 | 4829 | KLLTQDWVQENYLEYRQVPG | MAGE- |
| | | | 585] | | | | A10 [269- |
| | | | | | | | 288]; MAGE- |
| | | | | | | | A4 [245-264] |
| 10 | 3507 | KYHSDDYIKFLRSIRPDNMS | HDAC1 [66- | 9 | 4830 | APEEKIWEELSVLEVFEGRE | MAGE- |
| | | | 85] | | | | A3 [216- |
| | | | | | | | 235]; MAGE- |
| | | | | | | | A6 [216-235] |
| 10 | 3508 | SYKHLFQPVINQVVDFYQPT | HDAC3 [231- | 9 | 4831 | KSPQGASAIPTAIDFTLWRQ | MAGE-A5 [63- |
| | | | 250] | | | | 82] |
| 10 | 3509 | ISHATLERIGAVPVMVPAQS | IGFS11 [406- | 9 | 4832 | LRKLLTQEWVQENYLEYRQA | MAGE- |
| | | | 425] | | | | A8 [245-264] |
| 10 | 3510 | PQDFEIVDPGYLGYLYLQWQ | IL13RA2 [35- | 9 | 4833 | DHFTEILNGASRRLELVFGL | MAGE- |
| | | | 54] | | | | B1 [144-163] |
| 10 | 3511 | MNPNTLMTHEVPVYRTNQCA | JARID1B [556- | 9 | 4834 | KASEGLSVVFGLELNKVNPN | MAGE- |
| | | | 575] | | | | B2 [155-174] |
| 10 | 3512 | MYVIDLMFHPGGLMKLRSRE | KU-CT- | 9 | 4835 | ILKKASFNMEVVFGVDLKKV | MAGE- |
| | | | 1 [846-865] | | | | B3 [152-171] |
| 10 | 3513 | ISSIGLTYFQSSNLQCSTCT | LIPI [418- | 9 | 4836 | DTTPNNFPLLYEEALRDEEE | MAGE- |
| | | | 437] | | | | B4 [294-313] |
| 10 | 3514 | TGHSYVLVTCLGLSYDGLLG | MAGE- | 9 | 4837 | DSSGESYTLVSKLGLPSEGI | MAGE- |
| | | | A1 [165-184] | | | | B6 [256-275] |
| 10 | 3515 | AQDRIATTDDTTAMASASSS | MAGE- | 9 | 4838 | MIVIKYKDYFPVILKRAREF | MAGE- |
| | | | A10 [341- | | | | C2 [169-188] |
| | | | 360] | | | | |
| 10 | 3516 | EVLSIMGVYAGREHFLFGEP | MAGE- | 9 | 4839 | EKQRELKTARTLSLFYGVNV | MORC1 [341- |
| | | | A11 [336- | | | | 360] |
| | | | 355] | | | | |
| 10 | 3517 | MAELVHFLLLKYRAREPFTK | MAGE- | 9 | 4840 | TDYYQELQRDISEMFLQIYK | MUC-1 [1063- |
| | | | A12 [113- | | | | 1082] |
| | | | 132] | | | | |
| 10 | 3518 | SVLRNCQDFFPVIFSKASEY | MAGE- | 9 | 4841 | AETEPERHLGSYLLDSENTS | NKX3.1 [91- |
| | | | A2 [138-157] | | | | 110] |
| 10 | 3519 | ALVETSYVKVLHHMVKISGG | MAGE- | 9 | 4842 | RNKSVRYLDLSANVLKDEGL | NLRP4 [805- |
| | | | A3 [277-296] | | | | 824] |
| 10 | 3520 | SGGPRISYPLLHEWALREGE | MAGE- | 9 | 4843 | RSLDPQSYSLIHQLLSAEDL | NR6A1 [244- |
| | | | A6 [294-313] | | | | 263] |
| 10 | 3521 | MLGDGHSMPKAALLIIVLGV | MAGE- | 9 | 4844 | PNDYTKFVAEYFQERQMLGT | NYD- |
| | | | A9 [188-207] | | | | TSPG [185- |
| | | | | | | | 204] |
| 10 | 3522 | RNGLLMPLLGVIFLKGNSAT | MAGE- | 9 | 4845 | EAELARRSLAQDAPPLPVPG | NY-ESO- |
| | | | B1 [197-216] | | | | 1 [101-120] |
| 10 | 3523 | SVTKGEMLKIVGKRFREHFP | MAGE- | 9 | 4846 | SMQNYVQFLKSSYANVFGDG | ODF2 [792- |
| | | | B2 [131-150] | | | | 811] |
| 10 | 3524 | FLNMLGIYDGKRHLIFGEPR | MAGE- | 9 | 4847 | DVRVTKFKAPQYTMAARVEP | ODF3 [189- |
| | | | B4 [223-242] | | | | 208] |
| 10 | 3525 | IITEDLVQDKYVVYRQVCNS | MAGE- | 9 | 4848 | LHKEKYTLEQALLSASQEIE | PEPP2 [659- |
| | | | B6 [331-350] | | | | 678] |
| 10 | 3526 | PVTEAEMLMIVIKYKDYFPV | MAGE- | 9 | 4849 | IYTRRNYEAANSLIQNLFKV | PIWIL1 [499- |
| | | | C2 [161-180] | | | | 518] |
| 10 | 3527 | KLKMCFNQIQNTYMVQYEKK | MORC1 [861- | 9 | 4850 | LVGNIVITRYNNRTYRIDDV | PIWIL2 [413- |
| | | | 880] | | | | 432] |
| 10 | 3528 | SDVPFPFSAQSGAGVPGWGI | MUC-1 [1142- | 9 | 4851 | IRKTQKIVIKKREPLNFGIP | RCAS1 [102- |
| | | | 1161] | | | | 121] |
| 10 | 3529 | PLCSTFSDQSAYVSAIRDCF | NXF2 [331- | 9 | 4852 | GAIRKMREDRWAVIAKMDRK | SCP-1 [946- |
| | | | 350] | | | | 965] |
| 10 | 3530 | VLVKRKLVHDIIDLIYLNGL | NYC- | 9 | 4853 | LQKKIMMETQQQEIASVRKS | SCP3a [211- |
| | | | TSPG [401- | | | | 230] |
| | | | 420] | | | | |
| 10 | 3531 | SLLMWITQCFLPVFLAQPPS | NY-ESO- | 9 | 4854 | EKEKRTLLERKLSLENKLLQ | se57-1 [199- |
| | | | 1 [157-176] | | | | 218] |
| 10 | 3532 | PYPYCLCYSKRSRSCGLCDL | ODF1 [51-70] | 9 | 4855 | EVLEERTRIQFIRWSHTRIF | SPATA19 [106- |
| | | | | | | | 125] |
| 10 | 3533 | VVTFGIKHSDYMTPLLVDVE | ODF3 [235- | 9 | 4856 | PTGGSRLASSSEVLASIENI | SPO11 [35- |
| | | | 254] | | | | 54] |
| 10 | 3534 | FTCAILCYFNHKSFWSLILS | ODF4 [194- | 9 | 4857 | AFDDIATYFSKKEWKKMKYS | SSX-1 [24- |
| | | | 213] | | | | 43] |
| 10 | 3535 | VVLEAPFLVGIEGSLKGSTY | OIP5 [113- | 9 | 4858 | WEKMKASEKIFYVYMKRKYE | SSX-2 [37- |
| | | | 132] | | | | 56] |
| 10 | 3536 | EELDESYQKVIELFSVCTNE | PBK [284- | 9 | 4859 | WEKMKSSEKIVYVYMKLNYE | SSX-4 [37- |
| | | | 303] | | | | 56] |
| 10 | 3537 | TDGGLFLLADVSHKVIRNDC | PIWIL2 [370- | 9 | 4860 | NGDDAFVRRPRVGSQIPEKM | SSX-5 [2-21] |
| | | | 389] | | | | |
| 10 | 3538 | WPGFAISVSYFERKLLFSAD | PIWIL4 [243- | 9 | 4861 | NEGTSSIVMEIQKQIEKKEK | TAF7L [384- |
| | | | 262] | | | | 403] |
| 10 | 3539 | NNDVCVHFHELHLGLGCPPN | PLAC1 [45- | 9 | 4862 | AAVALVSSSAWSSALGSQTT | TAG-1 [15- |
| | | | 64] | | | | 34] |
| 10 | 3540 | SPYLGQMINLRRLLLSHIHA | PRAME [253- | 9 | 4863 | IMLLERSIMAKEGPLKVAQT | TEKT5 [374- |
| | | | 272] | | | | 393] |
| 10 | 3541 | GEVKRQIYVAAFTVQAAAET | PSMA [726- | 9 | 4864 | KDRTMNLQDIRYILKNDLKD | TEX14 [497- |
| | | | 745] | | | | 516] |
| 10 | 3542 | DMNIYELIRGRRYPLSEKKI | RAGE-1 [86- | 9 | 4865 | KDLRRHKIYGRKRRLTSQDS | TEX15 [933- |
| | | | 105] | | | | 952] |
| 10 | 3543 | PTPDNVLSAVTPELINLAGA | SAGE1 [404- | 9 | 4866 | SEIELLRSQMANERISMQNL | TSGA10 [548- |
| | | | 423] | | | | 567] |
| 10 | 3544 | VECTYISIDQVPRTYAIMIS | SCRN1 [52- | 9 | 4867 | ESEQAALGEEAVLLLDDIMA | TSPY1 [53- |
| | | | 71] | | | | 72] |
| 10 | 3545 | LCAALWILMKNPVLICLALS | SLCO6A1 [372- | 9 | 4868 | DSFQDYIKSYLEQASRIWSW | TYR [458-477] |
| | | | 391] | | | | |
| 10 | 3546 | DQRTLAAAAAAQQGFLFPPG | SOX-6 [275- | 9 | 4869 | KIESQRIISGRISGYISEAS | ZNF165 [201- |
| | | | 294] | | | | 220] |
| 10 | 3547 | IFYVYMKRKYEAMTKLGFKA | SSX-2 [46- | 10 | 4870 | KLSQKFTKVNFTEIQKLVLD | AFP [242-261] |
| | | | 65] | | | | |
| 10 | 3548 | DLPCVIESLRTLDKKTFYKT | TAF7L [155- | 10 | 4871 | DAMLRKLYNVMFAKKVPEHV | AKAP-3 [375- |
| | | | 174] | | | | 394] |
| 10 | 3549 | VTLECFAFGNPVPRIKWRKV | TAG-1 [257- | 10 | 4872 | EVDMLKIRSEFKRKYGKSLY | ANXA2 [297- |
| | | | 276] | | | | 316] |
| 10 | 3550 | LVMTKCRLEHELAIKANTLC | TEKT5 [443- | 10 | 4873 | PSRQTAIIVNPPPPEYINTK | BRDT [4-23] |
| | | | 462] | | | | |
| 10 | 3551 | MGTPRIQHLLILLVLGASLL | TEX101 [1- | 10 | 4874 | PAQLAAQMLGKVSSIHSDQS | CABYR [172- |
| | | | 20] | | | | 191] |
| 10 | 3552 | PKPHVSDHNRLLHLARPKAQ | THEG [290- | 10 | 4875 | TSKLQRLLAESRQMVTDLEL | CCDC62 [631- |
| | | | 309] | | | | 650] |
| 10 | 3553 | VFKEAFHDISHCLKAQMEKI | TMEM31 [100- | 10 | 4876 | LKTEENSFKRLMIVKKEKLA | CDCA1 [334- |
| | | | 119] | | | | 353] |
| 10 | 3554 | QYFSDLFNILDTAIIVILLL | TPTE [152- | 10 | 4877 | SPPASNMLKMEWSREVTTNA | CRISP2 [52- |
| | | | 171] | | | | 71] |
| 10 | 3555 | HREKFCYELTGEPLVCSMEG | TSP50 [301- | 10 | 4878 | ALLARKQGAGDSLIAGSAMS | CT45 [34-53] |
| | | | 320] | | | | |
| 10 | 3556 | PEKDKFFAYLTLAKHTISSD | TYR [129- | 10 | 4879 | TLQTVHFTSEAVELQDMSLL | CTCFL [81- |
| | | | 148] | | | | 100] |
| 10 | 3557 | PYSSDNLYQMTSQLECMTWN | WT1 [220- | 10 | 4880 | TDNNLAVYDLSRDILNNFPH | CXorf61 [53- |
| | | | 239] | | | | 72] |
| 10 | 3558 | EIHTKEQILELLVLEQFLTI | ZNF165 [81- | 10 | 4881 | LEGGHWLSEKRHRLQAIRDG | DKKL1 [184- |
| | | | 100] | | | | 203] |
| 11 | 3559 | VLCSQPVSILSPNTLKEIEA | ACRBP [121- | 10 | 4882 | SVDSFQIVLDPMLDFYSDKN | FAM46D [172- |
| | | | 140] | | | | 191] |
| 11 | 3560 | GSNRNFSVWLGASVVAHLST | ACTL8 [330- | 10 | 4883 | PNTKAEMEMSLAEELNHGRQ | FATE1 [6-25] |
| | | | 349] | | | | |
| 11 | 3561 | HHCHCNYLWDPPNCLIKGYG | ADAM29 [640- | 10 | 4884 | FERIMKYERLARILLQEIPI | FBXO39 [289- |
| | | | 659] | | | | 308] |
| 11 | 3562 | DEKQWHDVSVYLGLTNCPSS | CCDC62 [457- | 10 | 4885 | PVKGYELAVTQHQQLAEIDI | FSIP1 [306- |
| | | | 476] | | | | 325] |
| 11 | 3563 | SADKMQQLNAAHQEALMKLE | CDCA1 [148- | 10 | 4886 | MAASALRGLPVAGGGESSES | GASZ [1-20] |
| | | | 167] | | | | |
| 11 | 3564 | ARCMRRDFVKHLKKKLKRMI | CT45 [170- | 10 | 4887 | RNRPGMLVLTPTRELALQVE | HAGE [314- |
| | | | 189] | | | | 333] |
| 11 | 3565 | CAYGTRYLDDLCVKILREDK | CT46 [51-70] | 10 | 4888 | EMTKYHSDEYIKFLRSIRPD | HDAC2 [64- |
| | | | | | | | 83] |
| 11 | 3566 | GAGAAARAGEGLGLIQEAAS | CT47 [175- | 10 | 4889 | QVNTDSETAVVNVTYSSKDQ | KOC1 [112- |
| | | | 194] | | | | 131] |
| 11 | 3567 | CEKLNRFNSELVHEILCLEK | CTAGE2 [101- | 10 | 4890 | SILKNLRRVHPVSTMVKGLY | LDHC [262- |
| | | | 120] | | | | 281] |
| 11 | 3568 | LEERLESIISGAALMADSSC | CTNNA2 [303- | 10 | 4891 | LGFSPGPILPSTRKLYEKKL | LEMD1 [19- |
| | | | 322] | | | | 38] |
| 11 | 3569 | ESIQFCFIWRAISITPVHKS | CXorf48 [223- | 10 | 4892 | GSDPVRYEFLWGPRALAETS | MAGE- |
| | | | 242] | | | | A8 [266-285] |
| 11 | 3570 | RPPSVAPPPMVAEIPSAGTG | DBPC [186- | 10 | 4893 | EPRKLITQDLVKLKYLEYRQ | MAGE- |
| | | | 205] | | | | B3 [243-262] |
| 11 | 3571 | KATDSFHTELHPRVAFWIIK | DKKL1 [154- | 10 | 4894 | PVSSSFSYTLLSLFQSSPER | MAGE- |
| | | | 173] | | | | C1 [446-465] |
| 11 | 3572 | SSFYQPSLFPYYNNLYNCPQ | DMRT1 [211- | 10 | 4895 | NAPPAYEKLSAEQSPPPYSP | MART1 [99- |
| | | | 230] | | | | 118] |
| 11 | 3573 | QKARCKIPALPLPTILPPIN | DPPA2 [74- | 10 | 4896 | RAKRKLYTSEISSPIDISGQ | MPHOSPH1 [1775- |
| | | | 93] | | | | 1794] |
| 11 | 3574 | IVVVVIAVVAGIVVLVISRK | EpCAM [272- | 10 | 4897 | AFSHTQVIELERKFSHQKYL | NKX3.1 [130- |
| | | | 291] | | | | 149] |
| 11 | 3575 | QDIGACVALLSVRVYYKKCP | EPHA2 [183- | 10 | 4898 | FKDPKRAMEAFNLVRESEQL | NLRP4 [315- |
| | | | 202] | | | | 334] |
| 11 | 3576 | SYERRQILHLITMMALKVLG | FAM46D [315- | 10 | 4899 | STWQELILLSSLTVYSKQIF | NR6A1 [322- |
| | | | 334] | | | | 341] |
| 11 | 3577 | VSKPFGMLMLSIWILLFVCY | FMR1NB [65- | 10 | 4900 | PKKPSAFKPAIEMQNSVPNK | NY-BR- |
| | | | 84] | | | | 1 [461-480] |
| 11 | 3578 | QVKTIKELGGYVSNLRKICS | FTHL17 [142- | 10 | 4901 | KNYEGMIDNYKSQVMKTRLE | ODF2 [537- |
| | | | 161] | | | | 556] |
| 11 | 3579 | CGHLITAVQNVITELPVNSQ | GASZ [367- | 10 | 4902 | SIPQFPITSDSTISTLETPQ | PASD1 [668- |
| | | | 386] | | | | 687] |
| 11 | 3580 | QDEKECLKYQVPLPDSMKLE | GATA-3 [178- | 10 | 4903 | RSVPAGLTLQSVSPQSLQGK | PEPP2 [595- |
| | | | 197] | | | | 614] |
| 11 | 3581 | GSLGPLLDGTATLRLVKRQV | gp100 [452- | 10 | 4904 | RRSIAGFVASINEGMTRWFS | PIWIL1 [641- |
| | | | 471] | | | | 660] |
| 11 | 3582 | GDVGNYYYGQGHPMKPHRIR | HDAC1 [17- | 10 | 4905 | GSEGTVVQLLANHFRVISRP | PIWIL3 [119- |
| | | | 36] | | | | 138] |
| 11 | 3583 | GDIGNYYYGQGHPMKPHRIR | HDAC2 [18- | 10 | 4906 | FEGRTRYSRITGGMEAEVGE | PRSS55 [59- |
| | | | 37] | | | | 78] |
| 11 | 3584 | PDVGNFHYGAGHPMKPHRLA | HDAC3 [11- | 10 | 4907 | APRLAEYQAYIDFEMKIGDP | SART3 [307- |
| | | | 30] | | | | 326] |
| 11 | 3585 | MTSQRSPLAPLLLLSLHGVA | IGFS11 [1- | 10 | 4908 | ESKQLNVYEIKVNKLELELE | SCP-1 [632- |
| | | | 20] | | | | 651] |
| 11 | 3586 | TNYNLFYWYEGLDHALQCVD | IL13RA2 [167- | 10 | 4909 | GEVQNMLEGVGVDINKALLA | SCP3a [68- |
| | | | 186] | | | | 87] |
| 11 | 3587 | DAFKSDYFNMPVHMVPTELV | JARID1B [387- | 10 | 4910 | YESTSAHIIEETEYVKKIRT | se57-1 [23- |
| | | | 406] | | | | 42] |
| 11 | 3588 | SVPLWSGVNVAGVALKTLDP | LDHC [197- | 10 | 4911 | IAKPNNAYEFGQIINALSTR | SPAG1 [800- |
| | | | 216] | | | | 819] |
| 11 | 3589 | LLGDNQIMPKTGFLIIVLVM | MAGE- | 10 | 4912 | STAHSRIRKERPISLGIFPL | SPAG9 [190- |
| | | | A1 [182-201] | | | | 209] |
| 11 | 3590 | GILILILSIIFIEGYCTPEE | MAGE- | 10 | 4913 | QDIHVTRDVVKHHLSKSDLL | SPATA19 [81- |
| | | | A10 [225- | | | | 100] |
| | | | 244] | | | | |
| 11 | 3591 | HPRKLLMQDLVQENYLEYRQ | MAGE- | 10 | 4914 | DVLDRHRESLLAALRRGGRE | SPO11 [14- |
| | | | A2 [241-260] | | | | 33] |
| 11 | 3592 | VFGIELMEVDPIGHLYIFAT | MAGE- | 10 | 4915 | DNDHNRRIQVEHPQMTFGRL | SSX-1 [85- |
| | | | A3 [161-180] | | | | 104] |
| 11 | 3593 | LLGNNQIFPKTGLLIIVLGT | MAGE- | 10 | 4916 | FGRLQGIFPKIMPKKPAEEG | SSX-3 [101- |
| | | | A4 [190-209] | | | | 120] |
| 11 | 3594 | FGIELMEVDPIGHVYIFATC | MAGE- | 10 | 4917 | HRLRERKQLVVYEEISDPEE | SSX-4 [166- |
| | | | A6 [162-181] | | | | 185] |
| 11 | 3595 | RYFPVIFGKASEFMQVIFGT | MAGE- | 10 | 4918 | QIPEKMQKAFDDIAKYFSEK | SSX-5 [16- |
| | | | A9 [144-163] | | | | 35] |
| 11 | 3596 | KSGLLMSLLVVIFMNGNCAT | MAGE- | 10 | 4919 | KIEDLMEMVQKLQKVGSLEP | SYCE1 [32- |
| | | | B6 [284-303] | | | | 51] |
| 11 | 3597 | LQGEEFQSSLQSPVSICSSS | MAGE- | 10 | 4920 | TADELRMISSTFLNLPFQGI | TDRD1 [807- |
| | | | C1 [616-635] | | | | 826] |
| 11 | 3598 | KHLCYCLYRPRKYLYVTSSF | MORC1 [262- | 10 | 4921 | RITIGTLFSVLHERRSQFPV | TEX14 [328- |
| | | | 281] | | | | 347] |
| 11 | 3599 | LVLVCVLVALAIVYLIALAV | MUC-1 [1164- | 10 | 4922 | FRQPIFSQYASHQPLPQATY | TEX15 [2742- |
| | | | 1183] | | | | 2761] |
| 11 | 3600 | EVLAGLLTNNKKLTYLNVSC | NLRP4 [741- | 10 | 4923 | RRQLDETNDELAQIARERDI | TSGA10 [330- |
| | | | 760] | | | | 349] |
| 11 | 3601 | DMYIVQKYISNPLLIGRYKC | NYC- | 10 | 4924 | TPESAPEELLAVQVELEPVN | TSPY1 [104- |
| | | | TSPG [235- | | | | 123] |
| | | | 254] | | | | |
| 11 | 3602 | DHRQLQLSISSCLQQLSLLM | NY-ESO- | 11 | 4925 | AYPEKMRNRVLLELNSADLD | 5T4 [324-343] |
| | | | 1 [141-160] | | | | |
| 11 | 3603 | DPCNPCYPCGSRFSCRKMIL | ODF1 [231- | 11 | 4926 | DVAFLLVYREKSNYVGATFQ | ADAM2 [264- |
| | | | 250] | | | | 283] |
| 11 | 3604 | VMKTRLEADEVAAQLERCDK | ODF2 [550- | 11 | 4927 | LQTMKQEFLINLVKQKPQIT | AFP [545-564] |
| | | | 569] | | | | |
| 11 | 3605 | CSPGPRYNVNPKILRTGKDL | ODF3 [64-83] | 11 | 4928 | YDSDSWAEDLIVSALLLIQY | AKAP-3 [518- |
| | | | | | | | 537] |
| 11 | 3606 | TSAHVMSMGLLHFYKSRSCS | ODF4 [126- | 11 | 4929 | KEFADSISKGLMVYANQVAS | AKAP-4 [323- |
| | | | 145] | | | | 342] |
| 11 | 3607 | QGQRGHTSMKAVYVEPAAAA | PASD1 [213- | 11 | 4930 | AQRQDIAFAYQRRTKKELAS | ANXA2 [66- |
| | | | 232] | | | | 85] |
| 11 | 3608 | VSHKVIRNDCVLDVMHAIYQ | PIWIL2 [380- | 11 | 4931 | YNPPDHEVVTMARMLQDVFE | BRDT [351- |
| | | | 399] | | | | 370] |
| 11 | 3609 | DTVQRLTYCLCHMYYNLPGI | PIWIL3 [827- | 11 | 4932 | EYSDKTTQFPSVYAVPGTEQ | CABYR [114- |
| | | | 846] | | | | 133] |
| 11 | 3610 | SAGSGQSPMTVLCSIDWFMV | PLAC1 [18- | 11 | 4933 | KEAQEQEFLSLQEEFQKLEK | CAGE1 [477- |
| | | | 37] | | | | 496] |
| 11 | 3611 | LLFSVLLLLSLVTGTQLGPR | PRSS55 [2- | 11 | 4934 | TQYDLSEFENIGAIGLELWQ | CALR3 [287- |
| | | | 21] | | | | 306] |
| 11 | 3612 | STAPPWLRHMAAAGISSTIT | SAGE1 [646- | 11 | 4935 | NKHNELRKAVSPPASNMLKM | CRISP2 [42- |
| | | | 665] | | | | 61] |
| 11 | 3613 | PAEVGDLFYDCVDTEIKFFK | SCRN1 [395- | 11 | 4936 | SVRSRLYVGREKKLALMLSG | CTAGE5 [65- |
| | | | 414] | | | | 84] |
| 11 | 3614 | PIYLENQFILTPTVATTLAG | SLCO6A1 [407- | 11 | 4937 | DSKLAVSLAETTGLIKLEEE | CTCFL [166- |
| | | | 426] | | | | 185] |
| 11 | 3615 | GDNYPVQFIPSTMAAAAASG | SOX-6 [300- | 11 | 4938 | SGLINSNTDNNLAVYDLSRD | CXorf61 [46- |
| | | | 319] | | | | 65] |
| 11 | 3616 | AQNGCLYVHSSVAQWRKCLH | SPAG9 [976- | 11 | 4939 | HKDWIFSIAWISDTMAVSGS | DCAF12 [183- |
| | | | 995] | | | | 202] |
| 11 | 3617 | NDISCMLKVSRRSLHILSTS | SPO11 [155- | 11 | 4940 | LVIAEMMELGSRSRGASQKK | FATE1 [32- |
| | | | 174] | | | | 51] |
| 11 | 3618 | ISYVYMKRNYKAMTKLGFKV | SSX-1 [46- | 11 | 4941 | EDYFSHHLAVYNSPQFKKTM | FBXO39 [201- |
| | | | 65] | | | | 220] |
| 11 | 3619 | VMTKLGFKVTLPPFMRSKRA | SSX-4 [57- | 11 | 4942 | STEPGSFKVDTASNLNSGKE | FSIP1 [35- |
| | | | 76] | | | | 54] |
| 11 | 3620 | IIYVYMKRKYEAMTKLGFKA | SSX-5 [46- | 11 | 4943 | KMEEKYQLTARLNMEQLLQS | Glypican- |
| | | | 65] | | | | 3 [76-95] |
| 11 | 3621 | EILSKKQETLRILRLHCQEK | SYCE1 [100- | 11 | 4944 | IPEELVSMAERFKAHQQKRE | HAGE [613- |
| | | | 119] | | | | 632] |
| 11 | 3622 | ISNPGLFTSLGPPLRSTTCH | TDRD1 [152- | 11 | 4945 | QDTDTKITISPLQELTLYNP | KOC1 [304- |
| | | | 71] | | | | 323] |
| 11 | 3623 | LIIKAGTETAILATKGCIPE | TEX101 [65- | 11 | 4946 | AAYNKLLNNNLSLKYSQTGY | KU-CT- |
| | | | 84] | | | | 1 [542-561] |
| 11 | 3624 | ATSREFTNAYKLPLAVGPPS | TEX14 [779- | 11 | 4947 | LDGAQDSDDSEELNIILQGN | LEMD1 [58- |
| | | | 798] | | | | 77] |
| 11 | 3625 | QRFRGMLWFDLSLLPELVQC | TEX15 [2086- | 11 | 4948 | DGSFSFKLLNQLGMIEEPRL | LIPI [373- |
| | | | 2105] | | | | 392] |
| 11 | 3626 | HLFALSTLYFYKFFLPTILS | TMEM31 [126- | 11 | 4949 | LLTQNWVQEKYLVYRQVPGT | MAGE-A11 [358-377] |
| | | | 145] | | | | |
| 11 | 3627 | PSSRPRLLWQTPTTQTLPST | TSP50 [66- | 11 | 4950 | EEQETASSSSTLVEVTLREV | MAGE- |
| | | | 85] | | | | A12 [34-53] |
| 11 | 3628 | NLNSHLIRHQRIHTREKPYE | ZNF165 [410- | 11 | 4951 | VAELVRFLLRKYQIKEPVTK | MAGE- |
| | | | 429] | | | | A8 [116-135] |
| 12 | 3629 | NRKVSRMRCLQNETYSALSP | ACRBP [501- | 11 | 4952 | LNGASRRLELVFGLDLKEDN | MAGE- |
| | | | 520] | | | | B1 [150-169] |
| 12 | 3630 | QRVAPEMFFSPQVFEQPGPS | ACTL8 [252- | 11 | 4953 | NSATEEEIWEFLNMLGVYDG | MAGE- |
| | | | 271] | | | | B2 [216-235] |
| 12 | 3631 | YVGKFLLQIPRATIIYANIS | ADAM2 [549- | 11 | 4954 | SKMKVLEFWAKVNKTVPSAF | MAGE- |
| | | | 568] | | | | B3 [284-303] |
| 12 | 3632 | LLHCLGVFLSCSGHIQDEHP | ADAM29 [6- | 11 | 4955 | FRKVSQRTELVFGLALKEVN | MAGE- |
| | | | 25] | | | | B4 [151-170] |
| 12 | 3633 | LAVSVILRVAKGYQELLEKC | AFP [365- | 11 | 4956 | PHSSPPYYEFLWGPRAHSES | MAGE- |
| | | | 384] | | | | C2 [290-309] |
| 12 | 3634 | CPGSTMGYMAQSTQYEKCGG | AKAP-4 [570- | 11 | 4957 | QKFGDFLQHSPSILQSKAKK | MPHOSPH1 [1731- |
| | | | 589] | | | | 1750] |
| 12 | 3635 | QQDTKGDYQKALLYLCGGDD | ANXA2 [320- | 11 | 4958 | HQKYLSAPERAHLAKNLKLT | NKX3.1 [145- |
| | | | 339] | | | | 164] |
| 12 | 3636 | MISSKPRLVVPYGLKTLLEG | CABYR [1-20] | 11 | 4959 | SDEGMEVIERLIYLYHKFHQ | NR6A1 [362- |
| | | | | | | | 381] |
| 12 | 3637 | LIHCSGEMLKFTEKSLAKSI | CAGE1 [193- | 11 | 4960 | PSLSQEQQEMVQAFSAQSGM | NXF2 [566- |
| | | | 212] | | | | 585] |
| 12 | 3638 | ILHFKNKYHENKKLIRCKVD | CALR3 [147- | 11 | 4961 | IEVHNKASLTPLLLSITKRS | NY-BR- |
| | | | 166] | | | | 1 [109-128] |
| 12 | 3639 | LNFNVENSQELIQMYDSKME | CCDC62 [299- | 11 | 4962 | LKEKIVLTHNRLKSLMKILS | OIP5 [199- |
| | | | 318] | | | | 218] |
| 12 | 3640 | GDCEGVIFEGEPMYLNVGEV | CT46 [196- | 11 | 4963 | MQKLGFGTGVNVYLMKRSPR | PBK [35-54] |
| | | | 215] | | | | |
| 12 | 3641 | GFVPPPLAPIRGLLFPVDTR | CTAGE2 [659- | 11 | 4964 | YNVTSDYAVHPMSPVGRTSR | PEPP2 [128- |
| | | | 678] | | | | 147] |
| 12 | 3642 | PGFVPPPLAPIRGPLFPVDA | CTAGE5 [689- | 11 | 4965 | VGQSIHREPNLSLSNRLYYL | PIWIL1 [842- |
| | | | 708] | | | | 861] |
| 12 | 3643 | GCGIHAIELNPSRTLLATGG | DCAF12 [140- | 11 | 4966 | VAGSMGFNVDYPKIIKVQEN | PIWIL4 [509- |
| | | | 159] | | | | 528] |
| 12 | 3644 | LQFHAGQAWVPTTHRRMISL | DPPA2 [241- | 11 | 4967 | NLRRLLLSHIHASSYISPEK | PRAME [261- |
| | | | 260] | | | | 280] |
| 12 | 3645 | KFITSILYENNVITIDLVQN | EpCAM [179- | 11 | 4968 | EENSRLLQERGVAYINADSS | PSMA [436- |
| | | | 198] | | | | 455] |
| 12 | 3646 | TWDQVITLDQVLDEVIPIHG | FAM46D [10- | 11 | 4969 | KRRGPAYVMELPKLKLSGVV | RAGE-1 [339- |
| | | | 29] | | | | 358] |
| 12 | 3647 | YFKIWAFLDVSFVERILKSQ | FBXO39 [370- | 11 | 4970 | QVISVTFEKALNAGFIQATD | SART3 [397- |
| | | | 389] | | | | 416] |
| 12 | 3648 | ISLILVCLPIYCRSLFWRSE | FMR1NB [193- | 11 | 4971 | DSDPALQKVNFLPVLEQVGN | SCP-1 [63- |
| | | | 212] | | | | 82] |
| 12 | 3649 | SNLRKICSPEAGLAEYLFDK | FTHL17 [154- | 11 | 4972 | DVIEGKTAVIEKRRKKRSSA | SCP3a [41- |
| | | | 173] | | | | 60] |
| 12 | 3650 | AARDGHTQVVALLVAHGAEV | GASZ [156- | 11 | 4973 | EAQNKELKTQVALSSETPRT | se57-1 [289- |
| | | | 175] | | | | 308] |
| 12 | 3651 | RDGTGHYLCNACGLYHKMNG | GATA-3 [277- | 11 | 4974 | DLNKVILLDPSIIEAKMELE | SPAG1 [713- |
| | | | 296] | | | | 732] |
| 12 | 3652 | RLAQSYLKEPMIVYVGTLDL | HAGE [436- | 11 | 4975 | DIMRDRIEQVRRSISRLTDV | SPATA19 [134- |
| | | | 455] | | | | 153] |
| 12 | 3653 | LPYSEYFEYFAPDFTLHPDV | HDAC3 [323- | 11 | 4976 | PNRGNQVERPQMTFGRLQGI | SSX-2 [88- |
| | | | 342] | | | | 107] |
| 12 | 3654 | IFCIALILGAFFYWRSKNKE | IGFS11 [255- | 11 | 4977 | AQIPEKIQKAFDDIAKYFSK | SSX-3 [15- |
| | | | 274] | | | | 34] |
| 12 | 3655 | DLNKGIEAKIHTLLPWQCTN | IL13RA2 [96- | 11 | 4978 | KDKLKIDLLPDGRHAVVEVE | TAF7L [126- |
| | | | 115] | | | | 145] |
| 12 | 3656 | LYALPCVLSQTPLLKDLLNR | JARID1B [849- | 11 | 4979 | DFSTKSVFSKFAQLNLAAED | TAG-1 [214- |
| | | | 868] | | | | 233] |
| 12 | 3657 | PCDLPLRLLVPTQFVGAIIG | KOC1 [193- | 11 | 4980 | LQVRGAEASRLWASRLTDDS | TEKT5 [100- |
| | | | 212] | | | | 119] |
| 12 | 3658 | WLIHGYRPVGSIPLWLQNFV | LIPI [99- | 11 | 4981 | EDIPEISRLSISQKLPSTTM | THEG [97-116] |
| | | | 118] | | | | |
| 12 | 3659 | EEGPSTSCILESLFRAVITK | MAGE-A1 [85- | 11 | 4982 | MDVLLRVFVERRQQYFSDLF | TPTE [139- |
| | | | 104] | | | | 158] |
| 12 | 3660 | PQSAQIACSSPSVVASLPLD | MAGE- | 11 | 4983 | DDERMEQMSNMTLMKETIST | TSGA10 [155- |
| | | | A10 [84-103] | | | | 174] |
| 12 | 3661 | CIPEEVMWEVLSIMGVYAGR | MAGE- | 11 | 4984 | YEVEAYRRRHHNSSLNFFNW | TSPY1 [241- |
| | | | A11 [328- | | | | 260] |
| | | | 347] | | | | |
| 12 | 3662 | MVELVHFLLLKYRAREPVTK | MAGE- | 11 | 4985 | FLLHHAFVDSIFEQWLRRHR | TYR [386-405] |
| | | | A2 [113-132] | | | | |
| 12 | 3663 | EFQAALSRKVAELVHFLLLK | MAGE- | 11 | 4986 | GATLKGVAAGSSSSVKWTEG | WT1 [244-263] |
| | | | A3 [104-123] | | | | |
| 12 | 3664 | FPDIFSKASECMQVIFGIDV | MAGE- | 12 | 4987 | DERQNRSFEGMVVAALLAGR | 5T4 [188-207] |
| | | | A8 [150-169] | | | | |
| 12 | 3665 | RRKLLMPLLGVIFLNGNSAT | MAGE- | 12 | 4988 | QKATDIMDAMLRKLYNVMFA | AKAP-3 [368- |
| | | | B2 [200-219] | | | | 387] |
| 12 | 3666 | NCAREEEIWEFLNMLGIYDG | MAGE- | 12 | 4989 | ELEHLQTVKNISPLQILPPS | BRDT [743- |
| | | | B4 [213-232] | | | | 762] |
| 12 | 3667 | AYAETTKMRVLRVLADSSNT | MAGE- | 12 | 4990 | QEKRGAVYERVTTINQEIQK | CDCA1 [381- |
| | | | B6 [363-382] | | | | 400] |
| 12 | 3668 | LGLSNIKFRPGSVVVQLTLA | MUC-1 [1087- | 12 | 4991 | QLQVQREIVNKHNELRKAVS | CRISP2 [33- |
| | | | 1106] | | | | 52] |
| 12 | 3669 | DRFLYTFYFCCRELRELPPT | NLRP4 [180- | 12 | 4992 | MMQKPGSNAPVGGNVTSSFS | CT45 [84- |
| | | | 199] | | | | 103] |
| 12 | 3670 | MSVPPHYQYIPHLFSYSGHS | NR6A1 [217- | 12 | 4993 | QLLAERTKEQLFFVETMSGD | CTCFL [190- |
| | | | 236] | | | | 209] |
| 12 | 3671 | LRHTPYSIRCERRMKWHSED | NXF2 [76-95] | 12 | 4994 | ADMADVMRLLSHLKIVEEAL | CTNNA2 [141- |
| | | | | | | | 160] |
| 12 | 3672 | LLWKKIHRMVILTILAIAPS | NYC- | 12 | 4995 | VEEDKPHYGLRAIKVDVVPR | CXorf48 [80- |
| | | | TSPG [340- | | | | 99] |
| | | | 359] | | | | |
| 12 | 3673 | RRDIKKVDRELRQLRCIDEF | ODF1 [12-31] | 12 | 4996 | SMVENKLVELEHTLLSKGFR | CXorf61 [85- |
| | | | | | | | 104] |
| 12 | 3674 | LPFQWRITHSFRWMAQVLAS | ODF4 [66-85] | 12 | 4997 | VPPPRFRPRYRRPFRPRPRQ | DBPC [272- |
| | | | | | | | 291] |
| 12 | 3675 | LQEPCVAFNQQQLVQQEQHL | PASD1 [470- | 12 | 4998 | KMTDNKTGEVLISENVVASI | DKKL1 [108- |
| | | | 489] | | | | 127] |
| 12 | 3676 | LSHSPWAVKKINPICNDHYR | PBK [56-75] | 12 | 4999 | PQYSMALAADSASGEVGNPL | DMRT1 [229- |
| | | | | | | | 248] |
| 12 | 3677 | TYKLCHIYYNWPGVIRVPAP | PIWIL1 [812- | 12 | 5000 | GVPFRTVSEWLESIKMQQYT | EPHA2 [903- |
| | | | 831] | | | | 922] |
| 12 | 3678 | CKYAHKLAFLSGHILHHEPA | PIWIL2 [944- | 12 | 5001 | VAQTSLEEFNVLEMEVMRRQ | FATE1 [121- |
| | | | 963] | | | | 140] |
| 12 | 3679 | SEKILMQDHICQPVSAADWS | PIWIL4 [451- | 12 | 5002 | KHNQDFIKRNIELAKESRNP | FSIP1 [249- |
| | | | 470] | | | | 268] |
| 12 | 3680 | CKKLKIFAMPMQDIKMILKM | PRAME [210- | 12 | 5003 | ARRDLKVFGNFPKLIMTQVS | Glypican- |
| | | | 229] | | | | 3 [201-220] |
| 12 | 3681 | EGNYTLRVDCTPLMYSLVHN | PSMA [457- | 12 | 5004 | SYTWDFGDSSGTLISRALVV | gp100 [257- |
| | | | 476] | | | | 276] |
| 12 | 3682 | TVPPAFINMAATGVSSMSTR | SAGE1 [600- | 12 | 5005 | PYNDYFEYFGPDFKLHISPS | HDAC1 [329- |
| | | | 619] | | | | 348]; |
| | | | | | | | HDAC2 [330- |
| | | | | | | | 349] |
| 12 | 3683 | RNCPWTVALWSRYLLAMERH | SART3 [373- | 12 | 5006 | RQQTDMAVNWAGGLHHAKKS | HDAC2 [127- |
| | | | 392] | | | | 146] |
| 12 | 3684 | GVRCICSQLSLTTKMDAEHP | SCRN1 [183- | 12 | 5007 | LQEPSDLRAVLLINSKSYVS | KU-CT- |
| | | | 202] | | | | 1 [592-611] |
| 12 | 3685 | TLIFSGFSGVPIVLAMTRVV | SLCO6A1 [588- | 12 | 5008 | GETRLALVQRNVAIMKSIIP | LDHC [103- |
| | | | 607] | | | | 122] |
| 12 | 3686 | AAAQQGFLFPPGITYKPGDN | SOX-6 [283- | 12 | 5009 | AREPFTKAEMLGSVIRNFQD | MAGE-A12 |
| | | | 302] | | | | [126-145] |
| 12 | 3687 | SKAERFKMMLTLISKGQKEL | SPAG1 [877- | 12 | 5010 | DGREHTVYGEPRKLLTQDWV | MAGE- |
| | | | 896] | | | | A4 [233-252] |
| 12 | 3688 | RYDEEVVKELMPLVVAVLEN | SPAG9 [41- | 12 | 5011 | AEMLESVIKNYKRYFPVIFG | MAGE- |
| | | | 60] | | | | A9 [132-151] |
| 12 | 3689 | SFEAHHLTVPAIRWLGLLPS | SPO11 [297- | 12 | 5012 | ETTKMKVLEFLAKMNGATPR | MAGE- |
| | | | 316] | | | | B1 [279-298] |
| 12 | 3690 | HNRRIQVEHPQMTFGRLHRI | SSX-1 [88- | 12 | 5013 | LKKPQRALSTTTSVDVSYKK | MAGE-B3 [62- |
| | | | 107] | | | | 81] |
| 12 | 3691 | PNRGNQVEHPQMTFGRLQGI | SSX-5 [88- | 12 | 5014 | LQIPVSPSSSSTLLSLFQSF | MAGE- |
| | | | 107] | | | | C1 [233-252] |
| 12 | 3692 | KDLGEARTICEALQKELDSL | SYCE1 [72- | 12 | 5015 | KDYFPVILKRAREFMELLFG | MAGE- |
| | | | 91] | | | | C2 [175-194] |
| 12 | 3693 | TPWMDYEFRVIASNILGTGE | TAG-1 [676- | 12 | 5016 | LKSGSMRIGKDFILFTKKEE | MORC1 [103- |
| | | | 695] | | | | 122] |
| 12 | 3694 | LRLRETQDTLQLLVMTKCRL | TEKT5 [431- | 12 | 5017 | EENIGILPRTLNVLFDSLQE | MPHOSPH1 [166- |
| | | | 450] | | | | 185] |
| 12 | 3695 | FCNDKDSLSQFWEFSETTAS | TEX101 [113- | 12 | 5018 | RAHLAKNLKLTETQVKIWFQ | NKX3.1 [154- |
| | | | 132] | | | | 173] |
| 12 | 3696 | EHSSKLRHPYLLQLMAVCLS | TEX14 [297- | 12 | 5019 | SLVEKTPDEAASLVEGTSDK | NY-BR- |
| | | | 316] | | | | 1 [263-282] |
| 12 | 3697 | QRILTVDSFAASSTVPHCEQ | TEX15 [1357- | 12 | 5020 | FKLENERLKASFAPMEDKLN | ODF2 [504- |
| | | | 1376] | | | | 523] |
| 12 | 3698 | IPRSSLEYRASSRLKELAAP | THEG [219- | 12 | 5021 | KASQPSFSIKGRSKLGGFSD | ODF3 [155- |
| | | | 238] | | | | 174] |
| 12 | 3699 | PTILSLSFFILLVLLLLLFI | TMEM31 [141- | 12 | 5022 | DQASFTTSMEWDTQVVKGSS | OIP5 [29-48] |
| | | | 160] | | | | |
| 12 | 3700 | LVDVVYIFFDIKLLRNIPRW | TPTE [171- | 12 | 5023 | TEADRVIQRTNSMQQLEQWI | PEPP2 [399- |
| | | | 190] | | | | 418] |
| 12 | 3701 | PEAVARRWPWMVSVRANGTH | TSP50 [117- | 12 | 5024 | DVSHKLLRIETAYDFIKRTS | PIWIL3 [283- |
| | | | 136] | | | | 302] |
| 12 | 3702 | QALVCASAKEGTAFRMEAVQ | TSPY1 [27- | 12 | 5025 | QEAQPLQPSHFLDISEDWSL | PLAC1 [184- |
| | | | 46] | | | | 203] |
| 12 | 3703 | EAPAFLPWHRLFLLRWEQEI | TYR [203- | 12 | 5026 | LTSPSMEIKEVASIILHKDF | PRSS55 [129- |
| | | | 222] | | | | 148] |
| 12 | 3704 | RNQGYSTVTFDGTPSYGHTP | WT1 [145- | 12 | 5027 | YDPDERIAAHQALQHPYFQE | RAGE-1 [268- |
| | | | 164] | | | | 287] |
| 12 | 3705 | CSECGRAFSQSSNLSQHQRI | ZNF165 [458- | 12 | 5028 | FVPPRSSSSQVSAVKPQTLG | SCP-1 [10- |
| | | | 477] | | | | 29] |
| 13 | 3706 | MNPEAIHFSGVKIFSNCSFE | ADAM2 [338- | 12 | 5029 | RKRLEMYTKASLKTSNQKIE | SCP3a [89- |
| | | | 357] | | | | 108] |
| 13 | 3707 | TMRSGFMQNEITCRMEFEEI | ADAM29 [161- | 12 | 5030 | NLVQRMEKEKRTLLERKLSL | se57-1 [193- |
| | | | 180] | | | | 212] |
| 13 | 3708 | SILDSYQCTAEISLADLATI | AFP [30-49] | 12 | 5031 | EQVRRSISRLTDVSAQDFSM | SPATA19 [141- |
| | | | | | | | 160] |
| 13 | 3709 | GDLENAFLNLVQCIQNKPLY | ANXA2 [250- | 12 | 5032 | PNRGNQVQRPQMTFGRLQGI | SSX-3 [88- |
| | | | 269] | | | | 107] |
| 13 | 3710 | LQCSNLYLEKRVKELQMKIT | CAGE1 [326- | 12 | 5033 | KQKNEKLISLQEQLQRFLKK | TAF7L [443- |
| | | | 345] | | | | 462] |
| 13 | 3711 | LYPNPKPEVLHMIYMRALQI | CDCA1 [36- | 12 | 5034 | IISDVLIDEHLVLKSASPHK | TDRD1 [883- |
| | | | 55] | | | | 902] |
| 13 | 3712 | CGIAYCPNQDSLKYYYVCQY | CRISP2 [150- | 12 | 5035 | DKKSENIASLGESLAMKEKT | TSGA10 [278- |
| | | | 169] | | | | 297] |
| 13 | 3713 | VTSRLYVRREKKFAVALSGL | CTAGE2 [36- | 13 | 5036 | LADLSPFAFSGSNASVSAPS | 5T4 [154-173] |
| | | | 55] | | | | |
| 13 | 3714 | CFRQKQLLNAHFRKYHDANF | CTCFL [522- | 13 | 5037 | SPNTLKEIEASAEVSPTTMT | ACRBP [131- |
| | | | 541] | | | | 150] |
| 13 | 3715 | GVKLVRMAATQIDSLCPQVI | CTNNA2 [444- | 13 | 5038 | IERGRILNWEGVQYLWSFVL | ACTL8 [67- |
| | | | 463] | | | | 86] |
| 13 | 3716 | SFCGDYGMIDESIYFSSDVV | CXorf48 [45- | 13 | 5039 | PPSKKSFFYKEVFESRNGDY | AKAP-3 [234- |
| | | | 64] | | | | 253] |
| 13 | 3717 | ATVPATAAGVVAVVVPVPAG | DBPC [16-35] | 13 | 5040 | DIMEAMLKRLVSALIGEEKE | AKAP-4 [421- |
| | | | | | | | 440] |
| 13 | 3718 | VVHGRLLSADTKGWVRLQFH | DPPA2 [225- | 13 | 5041 | DEIEIDFETLKASTLRELEK | BRDT [550- |
| | | | 244] | | | | 569] |
| 13 | 3719 | MAPPQVLAFGLLLAAATATF | EpCAM [1-20] | 13 | 5042 | KTTSGMSKKSVESVKLAQLE | CABYR [350- |
| | | | | | | | 369] |
| 13 | 3720 | GMTKYDYLMTLHGVVNESTV | FAM46D [292- | 13 | 5043 | QDWEKHFLDASTSKQSDWNG | CALR3 [226- |
| | | | 311] | | | | 245] |
| 13 | 3721 | VLTKKFQVTMRGLLSCLSKS | FBXO39 [103- | 13 | 5044 | EKQDYKQKLKALKIEVNKLK | CCDC62 [204- |
| | | | 122] | | | | 223] |
| 13 | 3722 | RAMRVAHLELATYELAATES | FMR1NB [17- | 13 | 5045 | ESPDLSISHSQVEQLVNKTS | CT46 [306- |
| | | | 36] | | | | 325] |
| 13 | 3723 | PASSSSLSGGHASPHLFTFP | GATA-3 [135- | 13 | 5046 | TLEGERNQIYIQLSEVDKTK | CTAGE5 [328- |
| | | | 154] | | | | 347] |
| 13 | 3724 | LQVTRIFLQALNLGIEVINT | Glypican- | 13 | 5047 | SRDILNNFPHSIARQKRILV | CXorf61 [63- |
| | | | 3 [223-242] | | | | 82] |
| 13 | 3725 | FYTTDRVMTVSFHKYGEYFP | HDAC1 [187- | 13 | 5048 | VVDVQTSQITKIPILKDREP | DCAF12 [114- |
| | | | 206]; | | | | 133] |
| | | | HDAC2 [188- | | | | |
| | | | 207] | | | | |
| 13 | 3726 | NANIPSIYANGTHLVPGQHK | IGFS11 [351- | 13 | 5049 | EVLISENVVASIQPAEGSFE | DKKL1 [116- |
| | | | 370] | | | | 135] |
| 13 | 3727 | VIFVTGLLLRKPNTYPKMIP | IL13RA2 [355- | 13 | 5050 | GISHPIPLPSAAELLVKREN | DMRT1 [132- |
| | | | 374] | | | | 151] |
| 13 | 3728 | ENDQVVVKITGHFYACQVAQ | KOC1 [531- | 13 | 5051 | DLAPDTTYLVQVQALTQEGQ | EPHA2 [496- |
| | | | 550] | | | | 515] |
| 13 | 3729 | IQSAYEIIKLKGYTSWAIGL | LDHC [235- | 13 | 5052 | KAAGSASAKRVWNMTATRPK | FATE1 [57- |
| | | | 254] | | | | 76] |
| 13 | 3730 | FNTQKKTVWLIHGYRPVGSI | LIPI [91- | 13 | 5053 | SKTLGIGRLKRPSFLDDPLY | FSIP1 [524- |
| | | | 110] | | | | 543] |
| 13 | 3731 | EALNMMGLYDGMEHLIYGEP | MAGE- | 13 | 5054 | EAGLAEYLFDKLTLGGRVKE | FTHL17 [163- |
| | | | A10 [248- | | | | 182] |
| | | | 267] | | | | |
| 13 | 3732 | KVLHHLLKISGGPHISYPPL | MAGE- | 13 | 5055 | PVNSQKITLEWASPQNFTSV | GASZ [382- |
| | | | A12 [285- | | | | 401] |
| | | | 304] | | | | |
| 13 | 3733 | EQQTASSSSTLVEVTLGEVP | MAGE-A2 [35- | 13 | 5056 | MEVTVYHRRGSRSYVPLAHS | gp100 [184- |
| | | | 54] | | | | 203] |
| 13 | 3734 | AEMLGSVVGNWQYFFPVIFS | MAGE- | 13 | 5057 | KALENFKTGKVRILIATDLA | HAGE [528- |
| | | | A3 [133- | | | | 547] |
| | | | 152]; MAGE- | | | | |
| | | | A6 [133-152] | | | | |
| 13 | 3735 | WEAGMLMHFILRKYKMREPI | MAGE- | 13 | 5058 | QNSRQYLDQIRQTIFENLKM | HDAC3 [349- |
| | | | B1 [110-129] | | | | 368] |
| 13 | 3736 | VVFGLELNKVNPNGHTYTFI | MAGE- | 13 | 5059 | SIPVHLNSLPRLETLVAEVQ | JARID1B [1048- |
| | | | B2 [162-181] | | | | 1067] |
| 13 | 3737 | LCNDVLAMKRSSSLPSWKSL | MORC1 [757- | 13 | 5060 | THEDKIVRRNATMIFGILAS | KU-CT-1 [77- |
| | | | 776] | | | | 96] |
| 13 | 3738 | MTPGTQSPFFLLLLLTVLTV | MUC-1 [1-20] | 13 | 5061 | APEEEIWEELSVMEVYDGRE | MAGE- |
| | | | | | | | A1 [209-228] |
| 13 | 3739 | AELFALLCRLADELLFRQIA | NR6A1 [281- | 13 | 5062 | TVRPADLTRVIMPLEQRSQH | MAGE-A11 |
| | | | 300] | | | | [100-119] |
| 13 | 3740 | HENENYLLHENCMLKKEIAM | NY-BR- | 13 | 5063 | PVIFGKASESLKMIFGIDVK | MAGE- |
| | | | 1 [1038- | | | | A4 [149-168] |
| | | | 1057] | | | | |
| 13 | 3741 | SQALGSLRTTTPAFTLNIPS | NYC- | 13 | 5064 | TKAEMLESVIKNYKNHFPDI | MAGE- |
| | | | TSPG [12-31] | | | | A8 [134-153] |
| 13 | 3742 | ENRYDCLGSKKYSYMNICKE | ODF1 [155- | 13 | 5065 | EPRKLLTQDWVQENYLEYRQ | MAGE- |
| | | | 174] | | | | A9 [240-259] |
| 13 | 3743 | RTKAFRVDSTPGPAAYMLPM | ODF3 [127- | 13 | 5066 | QSRTDPLIMKTNMLVQFLME | MAGE- |
| | | | 146] | | | | B3 [105-124] |
| 13 | 3744 | KVTFIFSTLMLFPINIWIFE | ODF4 [151- | 13 | 5067 | TTAMTSAYSRATSSSSSQPM | MAGE- |
| | | | 170] | | | | B4 [327-346] |
| 13 | 3745 | KSSQRKLNWIPSFPTYDYFN | PASD1 [15- | 13 | 5068 | EYKPYFPQILNRTSQHLVVA | MAGE- |
| | | | 34] | | | | B6 [228-247] |
| 13 | 3746 | MARGLKYLHQEKKLLHGDIK | PBK [150- | 13 | 5069 | PLQSPVISFSSSTSLSPFSE | MAGE- |
| | | | 169] | | | | C1 [850-869] |
| 13 | 3747 | FVASINLTLTKWYSRVVFQM | PIWIL2 [760- | 13 | 5070 | EEEEEASSASSTLYLVFSPS | MAGE-C2 [34- |
| | | | 779] | | | | 53] |
| 13 | 3748 | MSLKGHLQSVTAPMGITMKP | PIWIL3 [523- | 13 | 5071 | KKIIETMSSSKLSNVEASKE | MPHOSPH1 [1749- |
| | | | 542] | | | | 1768] |
| 13 | 3749 | PSLSHCSQLTTLSFYGNSIS | PRAME [400- | 13 | 5072 | LSSELGDLEKHSSLPALKEE | NKX3.1 [184- |
| | | | 419] | | | | 203] |
| 13 | 3750 | VLPGLTYLTVAGIPAMSTRD | SAGE1 [554- | 13 | 5073 | KFKEHLKQMTLQLELKQIPW | NLRP4 [25- |
| | | | 573] | | | | 44] |
| 13 | 3751 | FIDENVATHSAGIYLGIAEC | SLCO6A1 [253- | 13 | 5074 | SETLKHLVLQFLQQYYSIYD | NXF2 [385- |
| | | | 272] | | | | 404] |
| 13 | 3752 | CWPTGGATVAEARVYRDARG | SOX-6 [594- | 13 | 5075 | ELSKSMESMRGHLQAQLRSK | ODF2 [337- |
| | | | 613] | | | | 356] |
| 13 | 3753 | KEACAHLLAITAPKDLPMFL | SPAG1 [822- | 13 | 5076 | IVNASEMDIQNVPLSEKIAE | OIP5 [179- |
| | | | 841] | | | | 198] |
| 13 | 3754 | AGSIYREFERLIGRYDEEVV | SPAG9 [28- | 13 | 5077 | YSPQRTYRSEVSSPIQRGDV | PEPP2 [556- |
| | | | 47] | | | | 575] |
| 13 | 3755 | YVYMKRKYEAMTKLGFKAIL | SSX-3 [48- | 13 | 5078 | LQFYNIIFRRLLKIMNLQQI | PIWIL1 [209- |
| | | | 67] | | | | 228] |
| 13 | 3756 | IKATKPLLMEQYCSIKIVDI | TDRD1 [396- | 13 | 5079 | DYPKIIKVQENPAAFVRAIQ | PIWIL4 [518- |
| | | | 415] | | | | 537] |
| 13 | 3757 | KCCGLTSLPAVQAPVIQECY | TEKT5 [17- | 13 | 5080 | LYSEELFPEELSVVLGTNDL | PRSS55 [110- |
| | | | 36] | | | | 129] |
| 13 | 3758 | EVKGCTAMIGCRLMSGILAV | TEX101 [181- | 13 | 5081 | SFDSLFSAVKNFTEIASKFS | PSMA [628- |
| | | | 200] | | | | 647] |
| 13 | 3759 | ILKKGIYVDAVNSLGQTALF | TEX14 [42- | 13 | 5082 | YVMELPKLKLSGVVRLSSYS | RAGE-1 [345- |
| | | | 61] | | | | 364] |
| 13 | 3760 | PTLRSLLWYDETLYAELLGK | TEX15 [1714- | 13 | 5083 | AIVEAARLEKVHSLFRRQLA | SART3 [237- |
| | | | 1733] | | | | 256] |
| 13 | 3761 | KRYVAYFAQVKHLYNWNLPP | TPTE [391- | 13 | 5084 | EETRQVYMDLNNNIEKMITA | SCP-1 [205- |
| | | | 410] | | | | 224] |
| 13 | 3762 | PRTSAPSRAGALLLLLLLLR | TSP50 [14- | 13 | 5085 | LQQSRIVQSQRLKTIKQLYE | SCP3a [161- |
| | | | 33] | | | | 180] |
| 13 | 3763 | YRVPERLRQGFCGVGRAAQA | TSPY1 [9-28] | 13 | 5086 | DRDEAWVLETIGKYWAAEKV | SCRN1 [161- |
| | | | | | | | 180] |
| 13 | 3764 | LAVLYCLLWSFQTSAGHFPR | TYR [3-22] | 13 | 5087 | KQEDSKQLLQVNKLEKEQKL | se57-1 [148- |
| | | | | | | | 167] |
| 14 | 3765 | YASWFESFCQFTHYRCSNHV | ACRBP [96- | 13 | 5088 | FLPITSSDIDVVESEAVSVL | SPATA19 [20- |
| | | | 115] | | | | 39] |
| 14 | 3766 | MAARTVIIDHGSGFLKAGTA | ACTL8 [1-20] | 13 | 5089 | ADSKMKAEIQALTFLSSDYL | SPO11 [362- |
| | | | | | | | 381]] |
| 14 | 3767 | SKPMRWPFFLFIPFFIIFCV | ADAM2 [682- | 13 | 5090 | LQIEEEKNKQRQLRLAFEEQ | SYCE1 [142- |
| | | | 701] | | | | 161] |
| 14 | 3768 | NIVDSILDVIGVKVLLFGLE | ADAM29 [230- | 13 | 5091 | GSIPGFLISSGMSSHKQGHT | TAF7L [295- |
| | | | 249] | | | | 314] |
| 14 | 3769 | LTSSELMAITRKMAATAATC | AFP [442- | 13 | 5092 | TDGRHFVSQTTGNLYIARTN | TAG-1 [179- |
| | | | 461] | | | | 198] |
| 14 | 3770 | TNSLQKQLQAVLQWIAASQF | AKAP-4 [767- | 13 | 5093 | EDTTLTITVPAVSRRVEELS | THEG [179- |
| | | | 786] | | | | 198] |
| 14 | 3771 | ASALKSALSGHLETVILGLL | ANXA2 [84- | 13 | 5094 | ENELDSAHSEIELLRSQMAN | TSGA10 [540- |
| | | | 103] | | | | 559] |
| 14 | 3772 | NGKSQYYIMFGPDICGFDIK | CALR3 [123- | 13 | 5095 | IQDVRRVPGVAPTLVRSASE | WT1 [297-316] |
| | | | 142] | | | | |
| 14 | 3773 | KALESNQMECQTALQKTQLQ | CCDC62 [94- | 13 | 5096 | EHGQEIFQKKVSPPGPALNV | ZNF165 [142- |
| | | | 113] | | | | 161] |
| 14 | 3774 | PRYNVAEIVIHIRNKILTGA | CDCA1 [7-26] | 14 | 5097 | GYHYRYEINADPRLTNLSSN | 5T4 [398-417] |
| 14 | 3775 | SREVTTNAQRWANKCTLQHS | CRISP2 [64- | 14 | 5098 | MDTSTDPVRVLSWLRRDLEK | AKAP-3 [32- |
| | | | 83] | | | | 51] |
| 14 | 3776 | EHQSLVLVKRLLAVSVSCIT | CT46 [23-42] | 14 | 5099 | PNHHQLAFNYQELEHLQTVK | BRDT [732- |
| | | | | | | | 751] |
| 14 | 3777 | GTGVQSTFTTFYEVDCDVID | CTNNA2 [810- | 14 | 5100 | DQAPEVTLQADIEVMSTVHI | CABYR [274- |
| | | | 829] | | | | 293] |
| 14 | 3778 | IHTEEVCITSVHGRNGVIDY | CXorf48 [175- | 14 | 5101 | SDEAKSIRDVPTLLGAKLDK | CAGE1 [674- |
| | | | 194] | | | | 693] |
| 14 | 3779 | FYLLLASSILCALIVFWKYR | CXorf61 [3- | 14 | 5102 | SKSLKSQVAEAKMTFKRFQA | CTAGE2 [150- |
| | | | 22] | | | | 169] |
| 14 | 3780 | RNYFSDIDFFPNAVYTHCYD | DCAF12 [408- | 14 | 5103 | AEAKMTFKIFQMNEERLKIA | CTAGE5 [188- |
| | | | 427] | | | | 207] |
| 14 | 3781 | QASGVRWCVVHGRLLSADTK | DPPA2 [217- | 14 | 5104 | AFQDSVLEEEVELVLAPSEE | CTCFL [55- |
| | | | 236] | | | | 74] |
| 14 | 3782 | GIAAGMKYLANMNYVHRDLA | EPHA2 [722- | 14 | 5105 | PQRPRNRPYFQRRRQQAPGP | DBPC [316- |
| | | | 741] | | | | 335] |
| 14 | 3783 | LHLITMMALKVLGELNILPN | FAM46D [322- | 14 | 5106 | LFSAPMDFRGLPGNYHKEEN | DKKL1 [71- |
| | | | 341] | | | | 90] |
| 14 | 3784 | LIIAVLVSASIANLWLWMNQ | FATE1 [164- | 14 | 5107 | VENTPDLVSDSTYYSSFYQP | DMRT1 [197- |
| | | | 183] | | | | 216] |
| 14 | 3785 | LPIYCRSLFWRSEPADDLQR | FMR1NB [200- | 14 | 5108 | TVNGEQLDLDPGQTLIYYVD | EpCAM [234- |
| | | | 219] | | | | 253] |
| 14 | 3786 | YTSYLYLSMAFYFNRDDVAL | FTHL17 [30- | 14 | 5109 | KYRKLIESELSYFVIVYSVM | FBXO39 [423- |
| | | | 49] | | | | 442] |
| 14 | 3787 | VSTWNSRILKRTAITICGFG | GASZ [443- | 14 | 5110 | DIEDVTPVFPQLSRSIISKL | FSIP1 [433- |
| | | | 462] | | | | 452] |
| 14 | 3788 | RAMLGTHTMEVTVYHRRGSR | gp100 [176- | 14 | 5111 | SSFNPAALSRHMSSLSHISP | GATA-3 [390- |
| | | | 195] | | | | 409] |
| 14 | 3789 | KIKNIQSTTNTTIQIIQEQP | HAGE [89- | 14 | 5112 | EAFEIVVRHAKNYTNAMFKN | Glypican- |
| | | | 108] | | | | 3 [113-132] |
| 14 | 3790 | EAIFKPVMSKVMEMFQPSAV | HDAC1 [238- | 14 | 5113 | GEARLREMEALQSLRLANEG | JARID1B [1146- |
| | | | 57] | | | | 1165] |
| 14 | 3791 | FCSRYTGASLQGATQLNNKI | HDAC3 [103- | 14 | 5114 | GGLKKLLSFAENSTIPDIQK | KU-CT- |
| | | | 122] | | | | 1 [274-293] |
| 14 | 3792 | MPATNVSIFINNTQLSDTGT | IGFS11 [98- | 14 | 5115 | GSDPRSFPLWYEEALKDEEE | MAGE-A10 [320-339] |
| | | | 117] | | | | |
| 14 | 3793 | WEGEDLSKKTLLRFWLPFGF | IL13RA2 [331- | 14 | 5116 | REDSGDFGLQVSTMFSEDDF | MAGE- |
| | | | 350] | | | | A11 [23-42] |
| 14 | 3794 | SMNLQAHLIPGLNLNALGLF | KOC1 [357- | 14 | 5117 | APEEKIWEELSMLEVFEGRE | MAGE- |
| | | | 376] | | | | A2 [216-235] |
| 14 | 3795 | VIGSGCNLDSARFRYLIGEK | LDHC [158- | 14 | 5118 | KASSSLQLVFGIELMEVDPI | MAGE- |
| | | | 177] | | | | A3 [153-172] |
| 14 | 3796 | LETNCNFISFPCRSYKDYKT | LIPI [285- | 14 | 5119 | AASSSSTLIMGTLEEVTDSG | MAGE-A8 [38- |
| | | | 304] | | | | 57] |
| 14 | 3797 | PARYEFLWGPRALAETSYVK | MAGE- | 14 | 5120 | EEPSSSVDPAQLEFMFQRAL | MAGE-A9 [89- |
| | | | A1 [259-278] | | | | 108] |
| 14 | 3798 | CAPEEKIWEELSVLEASDGR | MAGE- | 14 | 5121 | GEPRKFITQDLVQEKYLKYE | MAGE- |
| | | | A12 [215- | | | | B1 [239-258] |
| | | | 234] | | | | |
| 14 | 3799 | ELVTKAEMLERVIKNYKRCF | MAGE- | 14 | 5122 | AETSKMKVLEFLAKVNGTTP | MAGE- |
| | | | A4 [129-148] | | | | B2 [281-300] |
| 14 | 3800 | VAKLVHFLLLKYRAREPVTK | MAGE- | 14 | 5123 | NSNPARYEFLWGPRAHAETS | MAGE- |
| | | | A6 [113-132] | | | | B3 [265-284] |
| 14 | 3801 | AGARPRVAARRGTTAMTSAY | MAGE- | 14 | 5124 | PHLYEDALIDEVERALRLRA | MAGE- |
| | | | B4 [315-334] | | | | B6 [388-407] |
| 14 | 3802 | EEVIWEVLNAVGVYAGREHF | MAGE- | 14 | 5125 | SDEQGMSQNRLLILILSIIF | MAGE- |
| | | | C2 [246-265] | | | | C1 [989- |
| | | | | | | | 1008] |
| 14 | 3803 | KCGTLLVIYNLKLLLNGEPE | MORC1 [193- | 14 | 5126 | TSASGSASGSASTLVHNGTS | MUC-1 [959- |
| | | | 212] | | | | 978] |
| 14 | 3804 | TTQKEFFQGCIMQPVKDLLK | MPHOSPH1 [123- | 14 | 5127 | EAFSRASLVSVYNSYPYYPY | NKX3.1 [203- |
| | | | 142] | | | | 222] |
| 14 | 3805 | NRFPDLMMCLPEIRYIAGKM | NR6A1 [435- | 14 | 5128 | MAASFFSDFGLMWYLEELKK | NLRP4 [1-20] |
| | | | 454] | | | | |
| 14 | 3806 | QTERMLCFSVNGVFKEVEGQ | NXF2 [491- | 14 | 5129 | RADPVDLEFSVDQVDSVDQE | PASD1 [298- |
| | | | 510] | | | | 317] |
| 14 | 3807 | INLVDVYGNTALHYAVYSEI | NY-BR-1 [76- | 14 | 5130 | SATEVGRIQASPLPRSVDLS | PIWIL4 [17- |
| | | | 95] | | | | 36] |
| 14 | 3808 | FSDFKDFIFDDMYIVQKYIS | NYC- | 14 | 5131 | KYADKIYSISMKHPQEMKTY | PSMA [606- |
| | | | TSPG [225- | | | | 625] |
| | | | 244] | | | | |
| 14 | 3809 | IEAARRQFQSQLADLQQLPD | ODF2 [582- | 14 | 5132 | YVEIWQAYLDYLRRRVDFKQ | SART3 [417- |
| | | | 601] | | | | 436] |
| 14 | 3810 | TKPCAPVVTFGIKHSDYMTP | ODF3 [229- | 14 | 5133 | MEKQKPFALFVPPRSSSSQV | SCP-1 [1-20] |
| | | | 248] | | | | |
| 14 | 3811 | KSFWSLILSHPSGAVSCSSS | ODF4 [205- | 14 | 5134 | IEKRRKKRSSAGVVEDMGGE | SCP3a [50- |
| | | | 224] | | | | 69] |
| 14 | 3812 | PLGPAGLGAEEPAAGPQLPS | OIP5 [49-68] | 14 | 5135 | QDKRIENLREKVNILEAQNK | se57-1 [274- |
| | | | | | | | 293] |
| 14 | 3813 | GYRAFTEANDGSLCLAMEYG | PBK [99-118] | 14 | 5136 | LKNNLIEKDPSLVYQHLLYL | SPAG1 [857- |
| | | | | | | | 876] |
| 14 | 3814 | GILGSILLPSFQIALLTSED | PEPP2 [209- | 14 | 5137 | KTRDGGSVVGASVFYKDVAG | SPAG9 [699- |
| | | | 228] | | | | 718] |
| 14 | 3815 | LRDWGLSFDSNLLSFSGRIL | PIWIL1 [438- | 14 | 5138 | PDVENEVKRLLRSDAEAVST | TAF7L [259- |
| | | | 457] | | | | 278] |
| 14 | 3816 | TITSCEWVDFYLLAHHVRQG | PIWIL2 [880- | 14 | 5139 | WKRLGWSESSRIIVLDQSDL | TEX14 [1476- |
| | | | 899] | | | | 1495] |
| 14 | 3817 | TYCLCHMYYNLPGIIRVPAP | PIWIL3 [833- | 14 | 5140 | PSSRILQLSKPKAPATLLEE | THEG [264- |
| | | | 852] | | | | 283] |
| 14 | 3818 | TWKLPTLAKFSPYLGQMINL | PRAME [243- | 14 | 5141 | DLKIQIEMEKKVVFSTISLG | TPTE [430- |
| | | | 262] | | | | 449] |
| 14 | 3819 | SVLLLLSLVTGTQLGPRTPL | PRSS55 [5- | 14 | 5142 | KVITKEYLVNITEYRASHST | TSPY1 [214- |
| | | | 24] | | | | 233] |
| 14 | 3820 | PLLTTNLSPQCLSLLHAMVA | RAGE-1 [248- | 14 | 5143 | FINHQIIYAGEKNHQYGKSF | ZNF165 [307- |
| | | | 267] | | | | 326] |
| 14 | 3821 | RRFGQNYERIFILLEEVQGS | SAGE1 [836- | 15 | 5144 | EDVVLRWSQEFSTLTLGQFG | ACRBP [524- |
| | | | 855] | | | | 543] |
| 14 | 3822 | IHYFTGTPDPSRSIFKPFIF | SCRN1 [291- | 15 | 5145 | QKQSSYVGWWIHFRIVEIVV | ADAM29 [185- |
| | | | 310] | | | | 204] |
| 14 | 3823 | VIVSTLEMSCKALMRFIMVT | SLCO6A1 [442- | 15 | 5146 | ERQLNEAVGNVTPLQLLDWL | AKAP-3 [830- |
| | | | 461] | | | | 849] |
| 14 | 3824 | QIQVQGHMPPLMIPIFPHDQ | SOX-6 [257- | 15 | 5147 | VPHLQKVFDRYKSYSPYDML | ANXA2 [222- |
| | | | 276] | | | | 241] |
| 14 | 3825 | NCTCGATAVAVPSNIQGIRN | SPO11 [193- | 15 | 5148 | KNGRLTNQLQYLQKVVLKDL | BRDT [24-43] |
| | | | 212] | | | | |
| 14 | 3826 | AHRKHTMLQECKERISALNL | SYCE1 [123- | 15 | 5149 | APGHMSDVEWMSIFKPSKMQ | CCDC62 [525- |
| | | | 142] | | | | 544] |
| 14 | 3827 | RNGGTSMMVENMAVRPAPHP | TAG-1 [1002- | 15 | 5150 | LKEFEKTIHFYQKKIILHEK | CTAGE2 [408- |
| | | | 1021] | | | | 427] |
| 14 | 3828 | VIYSPGEFYCHVLKEDALKK | TDRD1 [722- | 15 | 5151 | SIYFSSDVVTGNVPLKVGQK | CXorf48 [56- |
| | | | 741] | | | | 75] |
| 14 | 3829 | QAPVIQECYQPYYLPGYRYL | TEKT5 [28- | 15 | 5152 | VPVKGSRYAPNRRKSRRFIP | DBPC [166- |
| | | | 47] | | | | 185] |
| 14 | 3830 | ADPANMFNWTTEEVETCDKG | TEX101 [38- | 15 | 5153 | DAQESSLGLTGLQSLLQGFS | DKKL1 [37- |
| | | | 57] | | | | 56] |
| 14 | 3831 | LICREDNAVSAATALLESEE | TEX15 [541- | 15 | 5154 | AISYRKFTSASDVWSFGIVM | EPHA2 [788- |
| | | | 560] | | | | 807] |
| 14 | 3832 | EQAQEEITRLRREMMKSCKS | TSGA10 [69- | 15 | 5155 | VESAVWYVKKFGRYLEHLEV | FBXO39 [75- |
| | | | 88] | | | | 94] |
| 14 | 3833 | KLKQELKYSNYVRPICLPGT | TSP50 [211- | 15 | 5156 | KRKRKSEMLQKAARGREEHG | FMR1NB [234- |
| | | | 230] | | | | 253] |
| 14 | 3834 | LYRNGDFFISSKDLGYDYSY | TYR [432- | 15 | 5157 | QGLEMRIKLWEEIKSAKYSE | FSIP1 [142- |
| | | | 451] | | | | 161] |
| 14 | 3835 | SDNHTTPILCGAQYRIHTHG | WT1 [273- | 15 | 5158 | LENFFRYFLRLSDDKMEHAQ | FTHL17 [49- |
| | | | 292] | | | | 68] |
| 15 | 3836 | GLPHIRVFLDNNPWVCDCHM | 5T4 [283- | 15 | 5159 | SKDQQKILAALKELQVEEIQ | GASZ [320- |
| | | | 302] | | | | 339] |
| 15 | 3837 | TLYPGFTKRLFRELMGDHVS | ACTL8 [302- | 15 | 5160 | KVKNQLRFLAELAYDLDVDD | Glypican- |
| | | | 321] | | | | 3 [515-534] |
| 15 | 3838 | PRTISLESLAVILAQLLSLS | ADAM2 [299- | 15 | 5161 | SQKRSFVYVWKTWGQYWQVL | gp100 [144- |
| | | | 318] | | | | 163] |
| 15 | 3839 | TSSYANRRPCFSSLVVDETY | AFP [502- | 15 | 5162 | NLLLNYGLYRKMEIYRPHKA | HDAC1 [40- |
| | | | 521] | | | | 59]; |
| | | | | | | | HDAC2 [41- |
| | | | | | | | 60] |
| 15 | 3840 | PDGECSIDDLSFYVNRLSSL | AKAP-4 [208- | 15 | 5163 | KKIRESYENDIASMNLQAHL | KOC1 [345- |
| | | | 227] | | | | 364] |
| 15 | 3841 | PSSEAAEDVMVAAPLVCSGK | CABYR [210- | 15 | 5164 | EALFKKSAETLWNIQKDLIF | LDHC [313- |
| | | | 229] | | | | 332] |
| 15 | 3842 | FKKIKANYVCLQERYMTEMQ | CAGE1 [410- | 15 | 5165 | KPPYSRLDYTDAKFVDVIHS | LIPI [213- |
| | | | 429] | | | | 232] |
| 15 | 3843 | SGTIFDNFLITDDEEYADNF | CALR3 [309- | 15 | 5166 | SFSQDILHDKIIDLVHLLLR | MAGE-A11 [216-235] |
| | | | 328] | | | | |
| 15 | 3844 | FLWQYKSSADKMQQLNAAHQ | CDCA1 [141- | 15 | 5167 | PRKLLTQDLVQENYLEYRQV | MAGE-Al2 [242-261] |
| | | | 160] | | | | |
| 15 | 3845 | KGLCTNSCQYQDLLSNCDSL | CRISP2 [198- | 15 | 5168 | GSNPARYEFLWGPRALAETS | MAGE- |
| | | | 217] | | | | A4 [264-283] |
| 15 | 3846 | KWMLGCYDALQKKYLRMVVL | CT46 [80-99] | 15 | 5169 | VVDEKYKDHFTEILNGASRR | MAGE- |
| | | | | | | | B1 [137-156] |
| 15 | 3847 | AFLSPPTLLEGPLRLSPLLP | CTAGE5 [533- | 15 | 5170 | TKGEMLKIVGKRFREHFPEI | MAGE- |
| | | | 552] | | | | B2 [133-152] |
| 15 | 3848 | RQSLQQCVAISIQQELYSPQ | CTCFL [127- | 15 | 5171 | KMKKPIMKADMLKIVQKSHK | MAGE- |
| | | | 146] | | | | B3 [127-146] |
| 15 | 3849 | RAARALLSAVTRLLILADMA | CTNNA2 [125- | 15 | 5172 | LKIISKKYKEHFPEIFRKVS | MAGE- |
| | | | 144] | | | | B4 [136-155] |
| 15 | 3850 | ILCALIVFWKYRRFQRNTGE | CXorf61 [11- | 15 | 5173 | LQIPVSRSFSSTLLSIFQSS | MAGE- |
| | | | 30] | | | | C1 [198-217] |
| 15 | 3851 | AVSLDGYFHLWKAENTLSKL | DCAF12 [269- | 15 | 5174 | ASSTLYLVFSPSSFSTSSSL | MAGE-C2 [42- |
| | | | 288] | | | | 61] |
| 15 | 3852 | GGSPVKNSLRGLPGPYVPGQ | DMRT1 [249- | 15 | 5175 | ERSQRSQIANITTVWRAQPT | MORC1 [668- |
| | | | 268] | | | | 687] |
| 15 | 3853 | RKRKAVTKRARLQRSYEMNE | DPPA2 [148- | 15 | 5176 | QKWREERDQLVAALEIQLKA | MPHOSPH1 [1516- |
| | | | 167] | | | | 1535] |
| 15 | 3854 | KDTEITCSERVRTYWIIIEL | EpCAM [129- | 15 | 5177 | ETQFNQYKTEAASRYNLTIS | MUC-1 [1118- |
| | | | 148] | | | | 1137] |
| 15 | 3855 | TQKVTCFYQPAPYFAAEARY | FAM46D [342- | 15 | 5178 | EKLDAFFGFQLSQEIKQQIH | NLRP4 [513- |
| | | | 361] | | | | 532] |
| 15 | 3856 | GHASPHLFTFPPTPPKDVSP | GATA-3 [144- | 15 | 5179 | EQLEERTWLLHDAIQNQQNA | PASD1 [382- |
| | | | 163] | | | | 401] |
| 15 | 3857 | KEPMIVYVGTLDLVAVSSVK | HAGE [443- | 15 | 5180 | RPRRINMTDTGISPMSTRDP | SAGE1 [226- |
| | | | 462] | | | | 245] |
| 15 | 3858 | RLFENLRMLPHAPGVQMQAI | HDAC2 [366- | 15 | 5181 | PIPESVIQNYNKALQQLEKY | SART3 [276- |
| | | | 385]; | | | | 295] |
| | | | HDAC1 [365- | | | | |
| | | | 384] | | | | |
| 15 | 3859 | DRVMTVSFHKYGNYFFPGTG | HDAC3 [185- | 15 | 5182 | EELKGTEQELIGLLQAREKE | SCP-1 [458- |
| | | | 204] | | | | 477] |
| 15 | 3860 | LYQGGQMFDGAPRFHGRVGF | IGFS11 [75- | 15 | 5183 | EGVGVDINKALLAKRKRLEM | SCP3a [75- |
| | | | 94] | | | | 94] |
| 15 | 3861 | TRQLCFVVRSKVNIYCSDDG | IL13RA2 [301- | 15 | 5184 | EIEALLGMDLVRLGLERGET | SCRN1 [103- |
| | | | 320] | | | | 122] |
| 15 | 3862 | CDVDKLHFTPRIQRLNELEA | JARID1B [76- | 15 | 5185 | IPPRDKMEDNSALYESTSAH | se57-1 [10- |
| | | | 95] | | | | 29] |
| 15 | 3863 | NDGTKIAASQAISAMCENSG | KU-CT- | 15 | 5186 | QAFPDMHNSNISKILGSRWK | SOX-6 [641- |
| | | | 1 [329-348] | | | | 660] |
| 15 | 3864 | VADLVGFLLLKYRAREPVTK | MAGE- | 15 | 5187 | PGNVKALLRRATTYKHQNKL | SPAG1 [273- |
| | | | A1 [106-125] | | | | 292] |
| 15 | 3865 | GMLSDVQSMPKTGILILILS | MAGE- | 15 | 5188 | QKVGSLEPRVEVLINRINEV | SYCE1 [44- |
| | | | A10 [213- | | | | 63] |
| | | | 232] | | | | |
| 15 | 3866 | REDSVFAHPRKLLMQDLVQE | MAGE- | 15 | 5189 | KVLLDAGFAVGEQSMVTDKP | TDRD1 [665- |
| | | | A2 [234-253] | | | | 684] |
| 15 | 3867 | KVLHHMVKISGGPHISYPPL | MAGE- | 15 | 5190 | QAKATQFNSALFTLSSHRQG | TEX14 [875- |
| | | | A3 [285-304] | | | | 894] |
| 15 | 3868 | LHHMVKISGGPRISYPLLHE | MAGE- | 15 | 5191 | SRRVEELSRPKRFYLEYYNN | THEG [191- |
| | | | A6 [287-306] | | | | 210] |
| 15 | 3869 | FLWGPRALAETSYVKVLEHV | MAGE- | 15 | 5192 | KVLKSERDKIFLLYEQAQEE | TSGA10 [55- |
| | | | A8 [274- | | | | 74] |
| | | | 293]; MAGE- | | | | |
| | | | A4 [272-291] | | | | |
| 15 | 3870 | LKVAELVHFLLHKYRVKEPV | MAGE- | 16 | 5193 | LIFLLNFTESRTLHRNEYGI | AFP [9-28] |
| | | | A9 [110-129] | | | | |
| 15 | 3871 | YDGILHSIYGDARKIITEDL | MAGE- | 16 | 5194 | QVASDMMVSLMKTLKVHSSG | AKAP-4 [339- |
| | | | B6 [317-336] | | | | 358] |
| 15 | 3872 | KMVNVPLEQLPLLFKVVLHS | NR6A1 [453- | 16 | 5195 | VNILTNRSNAQRQDIAFAYQ | ANXA2 [57- |
| | | | 472] | | | | 76] |
| 15 | 3873 | TLKIIERNFPELLSLNLCNN | NXF2 [261- | 16 | 5196 | SGKINRHEHYFNQFHRRNEL | CALR3 [365- |
| | | | 280] | | | | 384] |
| 15 | 3874 | ACLQRKMNVDVSSTIYNNEV | NY-BR- | 16 | 5197 | LQQSLNQDFHQKTIVLQEGN | CDCA1 [190- |
| | | | 1 [1173- | | | | 209] |
| | | | 1192] | | | | |
| 15 | 3875 | CKPAELSRGRGILIFSDFKD | NYC- | 16 | 5198 | PASNMLKMEWSREVTTNAQR | CRISP2 [54- |
| | | | TSPG [211- | | | | 73] |
| | | | 230] | | | | |
| 15 | 3876 | DELERKLEATSAQNIEFLQV | ODF2 [667- | 16 | 5199 | EELERTIHSYQGQIISHEKK | CTAGE5 [440- |
| | | | 686] | | | | 459] |
| 15 | 3877 | GPAYSILGRYQTKTMLTPGP | ODF3 [84- | 16 | 5200 | MSLLSIQQQEGVQVVVQQPG | CTCFL [97- |
| | | | 103] | | | | 116] |
| 15 | 3878 | RWMAQVLASELSLVAFILLL | ODF4 [77-96] | 16 | 5201 | QPFEENEFIDASRLVYDGVR | CTNNA2 [602- |
| | | | | | | | 621] |
| 15 | 3879 | STYNLLFCGSCGIPVGFHLY | OIP5 [130- | 16 | 5202 | GLINSNTDNNLAVYDLSRDI | CXorf61 [47- |
| | | | 149] | | | | 66] |
| 15 | 3880 | CSTPTINIPASPFMQKLGFG | PBK [22-41] | 16 | 5203 | LHKRKRLPPVKRSLVYYLKN | DCAF12 [33- |
| | | | | | | | 52] |
| 15 | 3881 | VTGQPSQDNCIFVVNEQTVA | PEPP2 [88- | 16 | 5204 | TLSSHLQIDKMTDNKTGEVL | DKKL1 [99- |
| | | | 107] | | | | 118] |
| 15 | 3882 | AWNSCNEYMPSRIIVYRDGV | PIWIL1 [685- | 16 | 5205 | DSKSLRTALQKEITTRYQLD | EpCAM [158- |
| | | | 704] | | | | 177] |
| 15 | 3883 | IAGPIGMRMSPPAWVELKDD | PIWIL2 [628- | 16 | 5206 | GDQDNWLRTNWVYRGEAERI | EPHA2 [75- |
| | | | 647] | | | | 94] |
| 15 | 3884 | KMSTYLKTISPNNFTLAFIV | PIWIL3 [741- | 16 | 5207 | FIKRNIELAKESRNPVVMVD | FSIP1 [254- |
| | | | 760] | | | | 273] |
| 15 | 3885 | ADCLKVFMTGALNKWYKYNH | PIWIL4 [663- | 16 | 5208 | IQNAAVFQEAFEIVVRHAKN | Glypican- |
| | | | 682] | | | | 3 [105-124] |
| 15 | 3886 | ELFPPLFMAAFDGRHSQTLK | PRAME [51- | 16 | 5209 | QVSLKVSNDGPTLIGANASF | gp100 [65- |
| | | | 70] | | | | 84] |
| 15 | 3887 | VIMDWEECSKMFPKLTKNML | PRSS55 [215- | 16 | 5210 | SDEYIKFLRSIRPDNMSEYS | HDAC2 [70- |
| | | | 234] | | | | 89] |
| 15 | 3888 | FKYHLTVAQVRGGMVFELAN | PSMA4570- | 16 | 5211 | RDYTLRTFGEMADAFKSDYF | JARID1B [375- |
| | | | 589] | | | | 394] |
| 15 | 3889 | APECLLTDGFYTYKMDLWSA | RAGE-1 [169- | 16 | 5212 | TSLGLTYDGMLSDVQSMPKT | MAGE-A10 [205-224] |
| | | | 188] | | | | |
| 15 | 3890 | GRCWIYNKTKMAFLLVGICF | SLCO6A1 [655- | 16 | 5213 | LEHVVRVNARVRIAYPSLRE | MAGE- |
| | | | 674] | | | | A4 [288-307] |
| 15 | 3891 | QRKRFTRVEMARVLMERNQY | SPAG9 [499- | 16 | 5214 | KASDSLQLVFGIELMEVDPI | MAGE- |
| | | | 518] | | | | A6 [153-172] |
| 15 | 3892 | DADPHGIEIMCIYKYGSMSM | SPO11 [277- | 16 | 5215 | QENYLEYRQAPGSDPVRYEF | MAGE- |
| | | | 296] | | | | A8 [255-274] |
| 15 | 3893 | CSEEYLERQLQAEFIESGQY | TAF7L [362- | 16 | 5216 | DGEEHLIYGEPRKFITQDLV | MAGE- |
| | | | 81] | | | | B1 [231-250] |
| 15 | 3894 | PGLSYRWLLNEFPNFIPTDG | TAG-1 [162- | 16 | 5217 | GEPWKLITKDLVQEKYLEYK | MAGE- |
| | | | 181] | | | | B2 [242-261] |
| 15 | 3895 | LECAANEVNCPLQVALECLY | TEKT5 [176- | 16 | 5218 | KVDSTKDSYVLVSKMDLPNN | MAGE- |
| | | | 195] | | | | B3 [170-189] |
| 15 | 3896 | VREACPHQLLTQPRKTENGA | TEX101 [205- | 16 | 5219 | AETSKMKVLEFLAKVNDTTP | MAGE- |
| | | | 224] | | | | B4 [278-297] |
| 15 | 3897 | KREKNSYYVFLKYKRQVNEC | TEX15 [1774- | 16 | 5220 | DDSTATESASSSVMSPSFSS | MAGE- |
| | | | 1793] | | | | C1 [1122- |
| | | | | | | | 1141] |
| 15 | 3898 | GLFGVFLVLLDVTLILADLI | TPTE [95- | 16 | 5221 | YTLDEKVAELVEFLLLKYEA | MAGE- |
| | | | 114] | | | | C2 [139-158] |
| 15 | 3899 | LQVSSYQHWIWDCLNGQALA | TSP50 [344- | 16 | 5222 | EQEKEEIASKSALLRQIKEV | MPHOSPH1 [207- |
| | | | 363] | | | | 226] |
| 15 | 3900 | CGVGRAAQALVCASAKEGTA | TSPY1 [20- | 16 | 5223 | DSLVERLHRQTAEYSAFKLE | ODF2 [488- |
| | | | 39] | | | | 507] |
| 15 | 3901 | DINIYDLFVWMHYYVSMDAL | TYR [169- | 16 | 5224 | EHGDSSAYENVKFIVNVRDI | PASD1 [118- |
| | | | 188] | | | | 137] |
| 15 | 3902 | FKDCERRFSRSDQLKRHQRR | WT1 [357- | 16 | 5225 | MDEAKILKSLHHPNIVGYRA | PBK [83-102] |
| | | | 376] | | | | |
| 15 | 3903 | ALSRLRELCCQWLKPEIHTK | ZNF165 [66- | 16 | 5226 | IPTSPSHGSIAAYQGYSPQR | PEPP2 [541- |
| | | | 85] | | | | 560] |
| 16 | 3904 | WVCDCHMADMVTWLKETEVV | 5T4 [296- | 16 | 5227 | HDIVNRQKSIAGFVASTNAE | PIWIL3 [657- |
| | | | 315] | | | | 676] |
| 16 | 3905 | QYPNYCSFKSQQCLMRNRNR | ACRBP [483- | 16 | 5228 | ARLVDNIQRNTNARFELETW | PIWIL4 [415- |
| | | | 502] | | | | 434] |
| 16 | 3906 | SGLLTGVVVDSGYGLTRVQP | ACTL8 [140- | 16 | 5229 | SISISALQSLLQHLIGLSNL | PRAME [417- |
| | | | 159] | | | | 436] |
| 16 | 3907 | TTGEANELLHTFLRWKTSYL | ADAM2 [239- | 16 | 5230 | PYEEALLQAEAPRLAEYQAY | SART3 [297- |
| | | | 258] | | | | 316] |
| 16 | 3908 | KHLPVFTYTDQGAILEDQPF | ADAM29 [76- | 16 | 5231 | EVEKAKVIADEAVKLQKEID | SCP-1 [681- |
| | | | 95] | | | | 700] |
| 16 | 3909 | AEDLIVSALLLIQYHLAQGG | AKAP-3 [524- | 16 | 5232 | SGKPSVEDQFTRAYDFETED | SCP3a [12- |
| | | | 543] | | | | 31] |
| 16 | 3910 | YCVQDTTSANTTLVHQTTPS | BRET [708- | 16 | 5233 | LKIKLQASREAGAAALRNVA | se57-1 [112- |
| | | | 727] | | | | 131] |
| 16 | 3911 | QLPEQIVIPFTDQVACLKEN | CABYR [311- | 16 | 5234 | MITQLISLREQLLAAHDEQK | SOX-6 [194- |
| | | | 330] | | | | 213] |
| 16 | 3912 | KSVSQYLEMDKTLSKKEEEV | CAGE1 [433- | 16 | 5235 | GQGENKMKNLPVPVYLRPLD | SPAG9 [660- |
| | | | 452] | | | | 679] |
| 16 | 3913 | QSDLQFLNFNVENSQELIQM | CCDC62 [293- | 16 | 5236 | QGHTSSEYDMLREMFSDSRS | TAF7L [311- |
| | | | 312] | | | | 330] |
| 16 | 3914 | SVSCITYLRGIFPECAYGTR | CT46 [37-56] | 16 | 5237 | PGNISWTFSSSSLSIKWDPV | TAG-1 [916- |
| | | | | | | | 935]] |
| 16 | 3915 | SLYPPTLLEGPLRLSPLLPR | CTAGE2 [503- | 16 | 5238 | PQAKEMFIDTVISSYNIETA | TEX15 [389- |
| | | | 522] | | | | 408] |
| 16 | 3916 | FCGDYGMIDESIYFSSDVVT | CXorf48 [46- | 16 | 5239 | TERDVAFTELRRMTTERDSL | TSGA10 [103- |
| | | | 65] | | | | 122] |
| 16 | 3917 | EAAAGATAVPAATVPATAAG | DBPC [5-24] | 16 | 5240 | TPIEWYPDYEVEAYRRRHHN | TSPY1 [233- |
| | | | | | | | 252]] |
| 16 | 3918 | CEPASEPSSFTVTPVIEEDE | DMRT1 [354- | 17 | 5241 | EDVRVSGWLQTEFLSFQDGD | ACRBP [452- |
| | | | 373] | | | | 471] |
| 16 | 3919 | ARAVQPKALNSCSIPVSVEA | DPPA2 [194- | 17 | 5242 | SNTYQLPDGSRVELTPMQRV | ACTL8 [235- |
| | | | 213] | | | | 254] |
| 16 | 3920 | PGNEEFQVVKDAVLDCLLDF | FAM46D [92- | 17 | 5243 | LFGLEIWTNKNLIVVDDVRK | ADAM29 [245- |
| | | | 111] | | | | 264] |
| 16 | 3921 | LLSCLSKSNNRLKSLSIQYL | FBXO39 [115- | 17 | 5244 | KWVESIFLIFLLNFTESRTL | AFP [2-21] |
| | | | 134] | | | | |
| 16 | 3922 | RRKAKGRNRRSHRAMRVAHL | FMR1NB [5- | 17 | 5245 | LGNGSSVDEVSFYANRLTNL | AKAP-3 [115- |
| | | | 24] | | | | 134] |
| 16 | 3923 | GDPQLCHFLESHYLHEQVKT | FTHL17 [126- | 17 | 5246 | SSGKPIPASVVLKRVLLRHT | AKAP-4 [356- |
| | | | 145] | | | | 375] |
| 16 | 3924 | EISGDEFLNFLLKLNKQCGH | GASZ [350- | 17 | 5247 | KIRSEFKRKYGKSLYYYIQQ | ANXA2 [302- |
| | | | 369] | | | | 321] |
| 16 | 3925 | ANGDPVCNACGLYYKLHNIN | GATA-3 [333- | 17 | 5248 | PDSQQQYNVVKTVKVTEQLR | BRET [255- |
| | | | 352] | | | | 274] |
| 16 | 3926 | TLCYLMPGFIHLVLQPSLKG | HAGE [293- | 17 | 5249 | AQLEENAKYSSVYMEAEATA | CABYR [366- |
| | | | 312] | | | | 385] |
| 16 | 3927 | CWTYETSLLVEEAISEELPY | HDAC3 [306- | 17 | 5250 | KMKNTDYLTQYDLSEFENIG | CALR3 [279- |
| | | | 325] | | | | 298] |
| 16 | 3928 | SESPGSIQVARGQPAVLPCT | IGFS11 [26- | 17 | 5251 | ARNETLSNTLVELSAQVGQL | CCDC62 [133- |
| | | | 45] | | | | 152] |
| 16 | 3929 | ESSCEIKLKWSIPLGPIPAR | IL13RA2 [249- | 17 | 5252 | SEQDELMADISKRIQSLEDE | CTAGE5 [160- |
| | | | 268] | | | | 179] |
| 16 | 3930 | TEEIPLKILAHNNFVGRLIG | KOC1 [274- | 17 | 5253 | EHQLGNNTLSSHLQIDKMTD | DKKL1 [92- |
| | | | 293] | | | | 111] |
| 16 | 3931 | PDVKKNSMECIYNLVQDFQC | KU-CT- | 17 | 5254 | QQLGLSTNGKKIEVYLRLHR | DPPA2 [105- |
| | | | 1 [164-183] | | | | 124] |
| 16 | 3932 | NSQCKITIVGTGAVGMACAI | LDHC [18-37] | 17 | 5255 | HKAREKPYDSKSLRTALQKE | EpCAM [150- |
| | | | | | | | 169] |
| 16 | 3933 | KLQEVKILAQFYNDFVNISS | LIPI [401- | 17 | 5256 | EQQKKIESYLHNHFIGEGMT | FAM46D [275- |
| | | | 420] | | | | 294] |
| 16 | 3934 | LESVIKNYKHCFPEIFGKAS | MAGE- | 17 | 5257 | GPLSVYPPASSSSLSGGHAS | GATA-3 [128- |
| | | | A1 [129-148] | | | | 147] |
| 16 | 3935 | WVQEKYLVYRQVPGTDPACY | MAGE- | 17 | 5258 | SLIYRRRLMKQDFSVPQLPH | gp100 [613- |
| | | | A11 [363- | | | | 632] |
| | | | 382] | | | | |
| 16 | 3936 | IGHLYILVTCLGLSYDGLLG | MAGE- | 17 | 5259 | QLSTGGSVASAVKLNKQQTD | HDAC1 [111- |
| | | | A12 [172- | | | | 130] |
| | | | 191] | | | | |
| 16 | 3937 | ISHLYILVTCLGLSYDGLLG | MAGE- | 17 | 5260 | HNLLLNYGLYRKMEIYRPHK | HDAC2 [40- |
| | | | A2 [172-191] | | | | 59] |
| 16 | 3938 | LGENQIMPKAGLLIIVLAII | MAGE- | 17 | 5261 | IREDDTTLVTATVENETYTL | IL13RA2 [276- |
| | | | A3 [190-209] | | | | 295] |
| 16 | 3939 | GTDVKEVDPAGHSYILVTAL | MAGE- | 17 | 5262 | QEGPSTFPDLETSFQVALSR | MAGE- |
| | | | A9 [162-181] | | | | A12 [92-111] |
| 16 | 3940 | CATEEEVWEFLGLLGIYDGI | MAGE- | 17 | 5263 | EEGPRMFPDLESEFQAAISR | MAGE-A2 [92- |
| | | | B6 [301-320] | | | | 111] |
| 16 | 3941 | AGALEDFPARWSFRAYTSVL | MORC1 [225- | 17 | 5264 | VGNWQYFFPVIFSKASSSLQ | MAGE- |
| | | | 244] | | | | A3 [140-159] |
| 16 | 3942 | KNYGQLDIFPARDTYHPMSE | MUC-1 [1189- | 17 | 5265 | EEGVEAQEEALGLVGAQAPT | MAGE-A4 [13- |
| | | | 1208] | | | | 32] |
| 16 | 3943 | TPQTQHQLKALCSLAAEGMW | NLRP4 [389- | 17 | 5266 | PAHLESLFREALDEKVAELV | MAGE- |
| | | | 408] | | | | A8 [101-120] |
| 16 | 3944 | MCLPEIRYIAGKMVNVPLEQ | NR6A1 [442- | 17 | 5267 | YKMREPIMKADMLKVVDEKY | MAGE- |
| | | | 461] | | | | B1 [123-142] |
| 16 | 3945 | RNWFKVTIPYGIKYDKAWLM | NXF2 [122- | 17 | 5268 | KSGSLVQFLLYKYKIKKSVT | MAGE- |
| | | | 141] | | | | B2 [114-133] |
| 16 | 3946 | IQDAQKRTALHWACVNGHEE | NY-BR-1 [12- | 17 | 5269 | VDVSYKKSYKGANSKIEKKQ | MAGE-B3 [75- |
| | | | 31] | | | | 94] |
| 16 | 3947 | KPLTIYVYQEGLVRFATEKF | NYC- | 17 | 5270 | SASQKAIIFKRLSKDAVKKK | MAGE- |
| | | | TSPG [266- | | | | B6 [179-198] |
| | | | 285] | | | | |
| 16 | 3948 | TWRPHRPRGPIMALYSSPGP | ODF3 [9-28] | 17 | 5271 | STFEGFAQSSLQIPVSPSFS | MAGE- |
| | | | | | | | C1 [258-277] |
| 16 | 3949 | RSCSDLENGKVTFIFSTLML | ODF4 [142- | 17 | 5272 | KLREILLYFFPEHQLPSELF | MORC1 [824- |
| | | | 161] | | | | 843] |
| 16 | 3950 | GHFCLSSDKMVCYLLKTKAI | OIP5 [160- | 17 | 5273 | QQHVPFRESKLTHYFQSFFN | MPHOSPH1 [426- |
| | | | 179] | | | | 445] |
| 16 | 3951 | KMGGELWRVDIPLKLVMIVG | PIWIL1 [611- | 17 | 5274 | QLKVLIAENTMLTSKLKEKQ | NY-BR- |
| | | | 630] | | | | 1 [1111- |
| | | | | | | | 1130] |
| 16 | 3952 | DSLKLCLVGSLKKFYEVNHC | PIWIL2 [786- | 17 | 5275 | ITPPSSEKLVSVMRLSDLST | ODF2 [91-110] |
| | | | 805] | | | | |
| 16 | 3953 | SLVNYNLWIEKVTQLEGRPF | PRSS55 [287- | 17 | 5276 | SMEWDTQVVKGSSPLGPAGL | OIP5 [36-55] |
| | | | 306] | | | | |
| 16 | 3954 | LHETDSAVATARRPRWLCAG | PSMA [5-24] | 17 | 5277 | GPPDPQAFQGPAAYQPDQMR | PASD1 [735- |
| | | | | | | | 754] |
| 16 | 3955 | PQCLSLLHAMVAYBPDERIA | RAGE-1 [256- | 17 | 5278 | GTGVNVYLMKRSPRGLSHSP | PBK [41-60] |
| | | | 275] | | | | |
| 16 | 3956 | PAPGNILSTAPPWLRHMAAA | SAGE1 [639- | 17 | 5279 | DVMHLSQSPSVSQLSVLSLS | PRAME [338- |
| | | | 658] | | | | 357] |
| 16 | 3957 | WGAEMGANEHGVCIANEAIN | SCRN1 [77- | 17 | 5280 | IGEGTFSEVMKMQSLREGNY | RAGE-1 [10- |
| | | | 96] | | | | 29] |
| 16 | 3958 | FLFAAVVAWCTLIPLSCFPN | SLCO6A1 [318- | 17 | 5281 | RKQDNVLSNVLSGLINMAGA | SAGE1 [451- |
| | | | 337] | | | | 470] |
| 16 | 3959 | YPSLWGFGTTKTFKIPIEHL | SPAG1 [6-25] | 17 | 5282 | TEKENKMKELTFLLEESRDK | SCP-1 [276- |
| | | | | | | | 295] |
| 16 | 3960 | TVVDNIINDISCMLKVSRRS | SPO11 [148- | 17 | 5283 | EYSQQFLTLFQQWELDMQKA | SCP3a [124- |
| | | | 167] | | | | 143] |
| 16 | 3961 | LKGERPGAAHQAGPDVLIGQ | SYCE1 [312- | 17 | 5284 | VAFPPRAKDGLVVFGKNSAR | SCRN1 [11- |
| | | | 331] | | | | 30] |
| 16 | 3962 | QPITSSHLALPFQIIRCSLE | TDRD1 [1039- | 17 | 5285 | IQDIITSLARNEAPAFTIDN | SPO11 [55- |
| | | | 1058] | | | | 74] |
| 16 | 3963 | EKCMGMRKTFPCTPRLVGHT | TEKT5 [466- | 17 | 5286 | GQDTSSQKIEDLMEMVQKLQ | SYCE1 [25- |
| | | | 485] | | | | 44] |
| 16 | 3964 | IGCRLMSGILAVGPMFVREA | TEX101 [189- | 17 | 5287 | DRGSETTYESSADIAGDEGT | TAF7L [36- |
| | | | 208] | | | | 55] |
| 16 | 3965 | PLPTQLYNWAAPEVILQKAA | TEX14 [414- | 17 | 5288 | LMELNGSSSQLIIMLLKNFM | TDRD1 [1060- |
| | | | 433] | | | | 1079] |
| 16 | 3966 | LCWPSVYWTERFLEDTTLTI | THEG [166- | 17 | 5289 | SNAAIQIASATMPALSLNND | TEX15 [598- |
| | | | 185] | | | | 617] |
| 16 | 3967 | NLPPRRILFIKHFIIYSIPR | TPTE [407- | 17 | 5290 | NEGYDRRPLASMSLPPPKAS | THEG [349- |
| | | | 426] | | | | 368] |
| 16 | 3968 | DQMTQTASDVPVLQVIMHSR | TSP50 [172- | 17 | 5291 | SSGRQSFYRNPIKEVVRFLD | TPTE [253- |
| | | | 191] | | | | 272] |
| 16 | 3969 | NLLSPASFFSSWQIVCSRLE | TYR [261- | 17 | 5292 | ELVRHHNMHQRNMTKLQLAL | WT1 [430-449] |
| | | | 280] | | | | |
| 16 | 3970 | KRPFMCAYPGCNKRYFKLSH | WT1 [320- | 17 | 5293 | SQSSNLSQHQRIHMRENLLM | ZNF165 [466- |
| | | | 339] | | | | 485] |
| 16 | 3971 | GTCDQSFKWNSDFINHQIIY | ZNF165 [295- | 18 | 5294 | PWPERLSNNVEELLQSSLSL | ACRBP [167- |
| | | | 314] | | | | 186] |
| 17 | 3972 | TGNQLAVLPAGAFARRPPLA | 5T4 [99-118] | 18 | 5295 | LAKDLIVSALKLIQYHLTQQ | AKAP-4 [540- |
| | | | | | | | 559] |
| 17 | 3973 | GSDTTVVAQKVFQLIGLTNA | ADAM2 [195- | 18 | 5296 | LEKDIISETSGDFRKLMVAL | ANXA2 [155- |
| | | | 214] | | | | 174] |
| 17 | 3974 | RQFETVCKFHWVEAFDDEMT | CAGE1 [119- | 18 | 5297 | DKSKLWLLKDRDLARQKEQE | BRET [900- |
| | | | 138] | | | | 919] |
| 17 | 3975 | SEKTKRLNELKLSVVSLKEI | CDCA1 [217- | 18 | 5298 | KVSSIHSDQSDVLMVDVATS | CABYR [182- |
| | | | 236] | | | | 201] |
| 17 | 3976 | SDPTSWSSAIQSWYDEILDF | CRISP2 [102- | 18 | 5299 | QERTNSELHNLRQIYVKQQS | CCDC62 [275- |
| | | | 121] | | | | 294] |
| 17 | 3977 | FEGEPMYLNVGEVSTPFHIF | CT46 [203- | 18 | 5300 | AQLQRTPMSALVFPNKISTE | CT46 [4-23] |
| | | | 222] | | | | |
| 17 | 3978 | PGPPRAPFAMRNVYLPRGFL | CTAGE2 [710- | 18 | 5301 | ELYQENEMKLHRKLTVEENY | cTAGE5 [387- |
| | | | 729] | | | | 406] |
| 17 | 3979 | KTFRTVTLLRNHVNTHTGTR | CTCFL [292- | 18 | 5302 | GPLKNTSDVINAAKKIAEAG | CTNNA2 [733- |
| | | | 311] | | | | 752] |
| 17 | 3980 | AGAIRGRAARVIHIINAEME | CTNNA2 [540- | 18 | 5303 | RLFLKGNLLRGIDSLFSAPM | DKKL1 [57- |
| | | | 559] | | | | 76] |
| 17 | 3981 | PSAPGSRTPGNPATAVSGTP | DBPC [58-77] | 18 | 5304 | VRTYWIIIELKHKAREKPYD | EpCAM [139- |
| | | | | | | | 158] |
| 17 | 3982 | NVCLAYGSEWSVYAVGSQAH | DCAF12 [299- | 18 | 5305 | VLPDGQVIWVNNTIINGSQV | gp100 [96- |
| | | | 318] | | | | 115] |
| 17 | 3983 | SFYQPSLFPYYNNLYNCPQY | DMRT1 [212- | 18 | 5306 | PARRAKRMRAEAMNIKIEPE | JARID1B [227- |
| | | | 231] | | | | 246] |
| 17 | 3984 | MELQAARACFALLWGCALAA | EPHA2 [1-20] | 18 | 5307 | LENFTLKVAYIPDEMAAQQN | KOC1 [144- |
| | | | | | | | 163] |
| 17 | 3985 | RILLQEIPIRSISLRSCYFS | FBXO39 [300- | 18 | 5308 | AAEADGIDPLINLLSSKRDG | KU-CT- |
| | | | 319] | | | | 1 [394-413] |
| 17 | 3986 | SLEEDSALEALLNFFFPTTC | FMR1NB [115- | 18 | 5309 | WKNIHKQVIQSAYEIIKLKG | LDHC [227- |
| | | | 134] | | | | 246] |
| 17 | 3987 | AENMKCLQFSKDVIISDTKD | FSIP1 [501- | 18 | 5310 | VKNTRKVAVSLSVHIKNLLK | LIPI [147- |
| | | | 520] | | | | 166] |
| 17 | 3988 | AINSHITLELYTSYLYLSMA | FTHL17 [20- | 18 | 5311 | KVLEYVIKVSARVRFFFPSL | MAGE- |
| | | | 39] | | | | A1 [278-297] |
| 17 | 3989 | LLDSGISVDSNFQYGWTPLM | GASZ [65-84] | 18 | 5312 | ASEEEIWEELGVMGVYDGRE | MAGE- |
| | | | | | | | A4 [217-236] |
| 17 | 3990 | RTACLVVAMLLSLDFPGQAQ | Glypican- | 18 | 5313 | VGNWQYFFPVIFSKASDSLQ | MAGE- |
| | | | 3 [6-25] | | | | A6 [140-159] |
| 17 | 3991 | VHDVTHVYNFDFPRNIEEYV | HAGE [553- | 18 | 5314 | VNPNGHTYTFIDKVDLTDEE | MAGE- |
| | | | 572] | | | | B2 [171-190] |
| 17 | 3992 | NNKICDIAINWAGGLHHAKK | HDAC3 [119- | 18 | 5315 | EFLNKMRIYDGKKHFIFGEP | MAGE- |
| | | | 138] | | | | B3 [225-244] |
| 17 | 3993 | NIGLIAGAIGTGAVIIIFCI | IGFS11 [239- | 18 | 5316 | GEPRKLITQDLVQEKYLEYQ | MAGE- |
| | | | 258] | | | | B4 [239-258] |
| 17 | 3994 | CAVNTFLTENSPYSLLEVLC | JARID1B [1072- | 18 | 5317 | RTSQHLVVAFGVELKEMDSS | MAGE- |
| | | | 1091] | | | | B6 [239-258] |
| 17 | 3995 | LIPGLNLNALGLFPPTSGMP | KOC1 [364- | 18 | 5318 | LDEKVDELARFLLLKYQVKQ | MAGE- |
| | | | 383] | | | | C1 [908-927] |
| 17 | 3996 | PEEEVVIHEFASLCLANMSA | KU-CT- | 18 | 5319 | AFTKLNNASSRSHSIFTVKI | MPHOSPH1 [340- |
| | | | 1 [119-138] | | | | 359] |
| 17 | 3997 | YSPDCKILVVSNPVDILTYI | LDHC [127- | 18 | 5320 | PSPGSTLSSSRSVELNGFMA | NR6A1 [190- |
| | | | 146] | | | | 209] |
| 17 | 3998 | NKRPCLEFSQLSVKDSFRDL | LIPI [38-57] | 18 | 5321 | YLKGELLRRTKRDIVDSLSA | NXF2 [454- |
| | | | | | | | 473] |
| 17 | 3999 | PVTKAEMLESVIKNYKHCFP | MAGE- | 18 | 5322 | IDIHFLERKMQHHLLKEKNE | NY-BR- |
| | | | A1 [122-141] | | | | 1 [1296- |
| | | | | | | | 1315] |
| 17 | 4000 | SDPARYEFLWGPRAHAEIRK | MAGE- | 18 | 5323 | EKSEEYAEQLHVQLADKDLY | ODF2 [414- |
| | | | A10 [289- | | | | 433] |
| | | | 308] | | | | |
| 17 | 4001 | LVHLLLRKYRVKGLITKAEM | MAGE- | 18 | 5324 | QPDQMRSAEQTRLMPAEQRD | PASD1 [749- |
| | | | A11 [229- | | | | 768] |
| | | | 248] | | | | |
| 17 | 4002 | MPKTGFLIIILAIIAKEGDC | MAGE- | 18 | 5325 | SNSIKRNPNAPVVRRGWLYK | PEPP2 [159- |
| | | | A6 [196-215] | | | | 178] |
| 17 | 4003 | GLSCDSMLGDGHSMPKAALL | MAGE- | 18 | 5326 | QETAQLVGSTASQQPGYIQP | PIWIL1 [16- |
| | | | A9 [182-201] | | | | 35] |
| 17 | 4004 | RNGLLMPLLSVIFLNGNCAR | MAGE- | 18 | 5327 | MSGRARVKARGIARSPSATE | PIWIL4 [1- |
| | | | B4 [197-216] | | | | 20] |
| 17 | 4005 | VTEAEMLMIVIKYKDYFPVI | MAGE- | 18 | 5328 | RRLWGSIQSRYISMSVWTSP | PRAME [4-23] |
| | | | C2 [162-181] | | | | |
| 17 | 4006 | SPGSGSSTTQGQDVTLAPAT | MUC-1 [65- | 18 | 5329 | RPFYRHVIYAPSSHNKYAGE | PSMA [684- |
| | | | 84] | | | | 703] |
| 17 | 4007 | EYISEMLLRNKSVRYLDLSA | NLRP4 [797- | 18 | 5330 | GSMKVKRQFVEFTIKEAARF | SAGE1 [854- |
| | | | 816] | | | | 873] |
| 17 | 4008 | RKQRNRCQYCRLLKCLQMGM | NR6A1 [106- | 18 | 5331 | QENRKIIEAQRKAIQELQFG | SCP-1 [135- |
| | | | 125] | | | | 154] |
| 17 | 4009 | NLCNNKLYQLDGLSDITEKA | NXF2 [276- | 18 | 5332 | MFRQQQKILQQSRIVQSQRL | SCP3a [153- |
| | | | 295] | | | | 172] |
| 17 | 4010 | QFIPLTFVMPNDYTKFVAEY | NYC- | 18 | 5333 | PLKNVRKKRFRKTQKKVPDV | TAF7L [225- |
| | | | TSPG [176- | | | | 244] |
| | | | 195] | | | | |
| 17 | 4011 | MTEEVWMGTWRPHRPRGPIM | ODF3 [1-20] | 18 | 5334 | KLVENSLSISNPGLFTSLGP | TDRD1 [144- |
| | | | | | | | 163] |
| 17 | 4012 | IFSTLMLFPINIWIFELERN | ODF4 [155- | 18 | 5335 | GEYFYSSTAQENLALETSSP | TEX14 [1088- |
| | | | 174] | | | | 1107] |
| 17 | 4013 | KKERPISMINEASNYNVTSD | PEPP2 [114- | 18 | 5336 | LKKAHRRVHTSLQLITKVGE | TEX15 [1004- |
| | | | 133] | | | | 1023] |
| 17 | 4014 | PLISVKPLDNWLLIYTRRNY | PIWIL1 [486- | 18 | 5337 | RKRRRRRRLMELAEPKINWQ | THEG [120- |
| | | | 505] | | | | 139] |
| 17 | 4015 | ERINLKNTSFITSQELNWVK | PIWIL2 [571- | 18 | 5338 | TTDKILIDVFDGLPLYDDVK | TPTE [458- |
| | | | 590] | | | | 477] |
| 17 | 4016 | PGIIRVPAPCHYAHKLAYLV | PIWIL3 [844- | 18 | 5339 | FWSWVGQANDIGLLKLKQEL | TSP50 [197- |
| | | | 863] | | | | 216] |
| 17 | 4017 | NENSEAQLAHLIPELCFLTG | PIWIL4 [365- | 18 | 5340 | ASPLTGIADASQSSMHNALH | TYR [348-367] |
| | | | 384] | | | | |
| 17 | 4018 | EPGEKWYQVGIISWGKSCGE | PRSS55 [259- | 18 | 5341 | ENSRSMPKLEIFEKIESQRI | ZNF165 [188- |
| | | | 278] | | | | 207] |
| 17 | 4019 | GAKGVILYSDPADYFAPGVK | PSMA4 [221- | 19 | 5342 | GFTKRLFRELMGDHVSSTKA | ACTL8 [306- |
| | | | 240] | | | | 325] |
| 17 | 4020 | LSSVGLHMTKGLALWEAYRE | SART3 [214- | 19 | 5343 | QITEEQLEAVIADFSGLLEK | AFP [562-581] |
| | | | 233] | | | | |
| 17 | 4021 | ENKLLQLKSSATYGKSCQDL | se57-1 [213- | 19 | 5344 | AQDKAESYSLISMKGMGDPK | AKAP-3 [397- |
| | | | 232] | | | | 416] |
| 17 | 4022 | LVAIFIAFYGDRKKVIWFVA | SLCO6A1 [157- | 19 | 5345 | LSGEAAEAVHSGTSVKSSSG | CABYR [451- |
| | | | 176] | | | | 470] |
| 17 | 4023 | VAEARVYRDARGRASSEPHI | SOX-6 [602- | 19 | 5346 | PVQEDMALNEVLQKLKHTNR | CAGE1 [298- |
| | | | 621] | | | | 317] |
| 17 | 4024 | ADGNVKAFYRRALAHKGLKN | SPAG1 [687- | 19 | 5347 | TILSPKQIKTPFQKILREKD | CT46 [239- |
| | | | 706] | | | | 258] |
| 17 | 4025 | DSTLRLYHAHTYQHLQDVDI | SPAG9 [1109- | 19 | 5348 | VAEAKMTFKRFQANEERLEI | CTAGE2 [157- |
| | | | 1128] | | | | 176] |
| 17 | 4026 | TATRRKPHLLLVAAVALVSS | TAG-1 [3-22] | 19 | 5349 | AAPIHDADAQESSLGLTGLQ | DKKL1 [30- |
| | | | | | | | 49] |
| 17 | 4027 | NSWRPSLFYKIANVQTCPDE | TEKT5 [48- | 19 | 5350 | ASGALVGAASGSSAGGSSRG | DMRT1 [34- |
| | | | 67] | | | | 53] |
| 17 | 4028 | KTENGATCLPIPVWGLQLLL | TEX101 [219- | 19 | 5351 | TRNDWRVASELINILERANQ | HAGE [592- |
| | | | 238] | | | | 611] |
| 17 | 4029 | GQPSLCSFEINEIYSGCLIL | TEX14 [619- | 19 | 5352 | LPEIQELYQTLLAKPSPAQQ | JARID1B [1360- |
| | | | 638] | | | | 1379] |
| 17 | 4030 | SCKSPKSTTAHAILRRVETE | TSGA10 [85- | 19 | 5353 | SDLESIFKDAKIPVSGPFLV | KOC1 [16-35] |
| | | | 104] | | | | |
| 17 | 4031 | KVDPYRSCGFSYEQDPTLRD | TSP50 [97- | 19 | 5354 | SSKREGAIANAATVLTNMAM | KU-CT- |
| | | | 116] | | | | 1 [408-427] |
| 17 | 4032 | FSDHNFAGSNKIAEILCKEL | TSPY1 [261- | 19 | 5355 | DLQHGSLFFSTSKITSGKDY | LDHC [64-83] |
| | | | 280] | | | | |
| 17 | 4033 | VGAVLTALLAGLVSLLCRHK | TYR [484- | 19 | 5356 | DMNVIVVEWSRGATTFIYNR | LIPI [126- |
| | | | 503] | | | | 145] |
| 18 | 4034 | LALIGAIFLLVLYLNRKGIK | 5T4 [363- | 19 | 5357 | WVQENYLEYRQVPGSNPARY | MAGE- |
| | | | 82] | | | | A4 [251-270] |
| 18 | 4035 | ISLRPLLVSHVMACGGNTLY | ACTL8 [285- | 19 | 5358 | SVMGLYDGREHSVYWKLRKL | MAGE- |
| | | | 304] | | | | A8 [229-248] |
| 18 | 4036 | IFCVLIAIMVKVNFQRKKWR | ADAM2 [698- | 19 | 5359 | NLSNDWDFPRNGLLMPLLGV | MAGE- |
| | | | 717] | | | | B1 [188-207] |
| 18 | 4037 | FGGQKHIIHIKVKKLLFSKH | ADAM29 [58- | 19 | 5360 | RGQTQDHQGAQITATNKKKV | MAGE-B3 [19- |
| | | | 77] | | | | 38] |
| 18 | 4038 | QHACAVMKNFGTRTFQAITV | AFP [221- | 19 | 5361 | RTHSTFEGFPQSLLQIPMTS | MAGE- |
| | | | 240] | | | | C1 [325-344] |
| 18 | 4039 | EVVSDLIDSFLRNLHSVTGT | AKAP-3 [328- | 19 | 5362 | KQEFLNVQEYNHLLKVMGQY | MORC1 [414- |
| | | | 347] | | | | 433] |
| 18 | 4040 | KLDKYHSLNEELDFLVTSYE | CAGE1 [690- | 19 | 5363 | VQQIQSNYDIAIAELHVQKS | MPHOSPH1 [933- |
| | | | 709] | | | | 952] |
| 18 | 4041 | CGFDIKKVHVILHFKNKYHE | CALR3 [137- | 19 | 5364 | TMNKRYNVSQQALDLQNLRF | NXF2 [220- |
| | | | 156] | | | | 239] |
| 18 | 4042 | PFSNLVTHLDSFLPICRVND | CDCA1 [84- | 19 | 5365 | YWRTSSFRMTEHNSVKPWQQ | NYC- |
| | | | 103] | | | | TSPG [124- |
| | | | | | | | 143] |
| 18 | 4043 | RAAVAGFFAVLFLWRSFRSV | CTAGE2 [17- | 19 | 5366 | NRSLGQRQNSPLPFQWRITH | ODF4 [55-74] |
| | | | 36] | | | | |
| 18 | 4044 | CCQCSYASRETYKLKRHMRT | CTCFL [372- | 19 | 5367 | RSWTYGITRGGRVFFINEEA | PEPP2 [14- |
| | | | 391] | | | | 33] |
| 18 | 4045 | WCPPPFFYRRRFVRGPRPPN | DBPC [223- | 19 | 5368 | KVLRSETVLDFMFNFYHQTE | PIWIL1 [272- |
| | | | 242] | | | | 291] |
| 18 | 4046 | GTLNKVFASQWLNHRQVVCG | DCAF12 [87- | 19 | 5369 | FKRANMENDIALLLLASPIK | PRSS55 [148- |
| | | | 106] | | | | 167] |
| 18 | 4047 | PQGRAGGFGKASGALVGAAS | DMRT1 [24- | 19 | 5370 | RLQDFDKSNPIVLRMMNDQL | PSMA [649- |
| | | | 43] | | | | 668] |
| 18 | 4048 | ALNSCSIPVSVEAFLMQASG | DPPA2 [201- | 19 | 5371 | KMENVQPAPDNVLLTLRPRR | SAGE1 [210- |
| | | | 220] | | | | 229] |
| 18 | 4049 | LEPHMNYTFTVEARNGVSGL | EPHA2 [402- | 19 | 5372 | AATEAPKMSNADFAKLFLRK | SART3 [944- |
| | | | 421] | | | | 963] |
| 18 | 4050 | EIRFTNLTWDQVITLDQVLD | FAM46D [3- | 19 | 5373 | GGSGAVMSERVSGLAGSIYR | SPAG9 [14- |
| | | | 22] | | | | 33] |
| 18 | 4051 | EVKFMNPYNAVLTKKFQVTM | FBXO39 [93- | 19 | 5374 | NEVKRLLRSDAEAVSTRWEV | TAF7L [263- |
| | | | 112] | | | | 282] |
| 18 | 4052 | FKCFAPFRDVPKQMMQMFGL | FMR1NB [171- | 19 | 5375 | NESVRPYTPFEVKIRSYNRR | TAG-1 [775- |
| | | | 190] | | | | 794] |
| 18 | 4053 | SEECEASKGYYLTKALTGHN | FSIP1 [473- | 19 | 5376 | FQAENTIMLLERSIMAKEGP | TEKT5 [368- |
| | | | 492] | | | | 387] |
| 18 | 4054 | SHYLHEQVKTIKELGGYVSN | FTHL17 [136- | 19 | 5377 | AYDPKHFHNRVVRIMIDDHN | TPTE [289- |
| | | | 155] | | | | 308] |
| 18 | 4055 | ANASFEKDKQSILITACSAH | GASZ [103- | 19 | 5378 | LVERREEAQRAQQAVPGPGP | TSPY1 [83- |
| | | | 122] | | | | 102] |
| 18 | 4056 | PTHHGSQVCRPPLLHGSLPW | GATA-3 [77- | 19 | 5379 | AQYRIHTHGVFRGIQDVRRV | WT1 [284-303] |
| | | | 96] | | | | |
| 18 | 4057 | PDATCHQVRSFFQRLQPGLK | Glypican- | 20 | 5380 | GRRDARSFVEAAGTTNFPAN | AKAP-3 [543- |
| | | | 3 [31-50] | | | | 562] |
| 18 | 4058 | QSQAWPIVLQGIDLIGVAQT | HAGE [269- | 20 | 5381 | SKIASEMAYEAVELTAAEMR | AKAP-4 [268- |
| | | | 288] | | | | 287] |
| 18 | 4059 | HSDDYIKFLRSIRPDNMSEY | HDAC1 [68- | 20 | 5382 | MKGKGTRDKVLIRIMVSRSE | ANXA2 [278- |
| | | | 87] | | | | 297] |
| 18 | 4060 | YGQGHPMKPHRIRMTHNLLL | HDAC2 [25- | 20 | 5383 | SATEKVFKQQEIPSVFPKTS | BRDT [166- |
| | | | 44] | | | | 185] |
| 18 | 4061 | LHHAKKFEASGFCYVNDIVI | HDAC3 [133- | 20 | 5384 | IKIGSEKSLHLEVEITSIVS | CABYR [409- |
| | | | 152] | | | | 428] |
| 18 | 4062 | TFTTSAALINLNVIWMVTPL | IGFS11 [45- | 20 | 5385 | ERPEIVSTWSSAGISWRSEA | CAGE1 [260- |
| | | | 64] | | | | 279] |
| 18 | 4063 | GFILILVIFVTGLLLRKPNT | IL13RA2 [349- | 20 | 5386 | HKEKDKGLQTTQNGRFYAIS | CALR3 [59- |
| | | | 368] | | | | 78] |
| 18 | 4064 | VQFLLFKYQMKEPITKAEIL | MAGE- | 20 | 5387 | NQKSLFKDQKFEAMLVQQNR | CCDC62 [348- |
| | | | A10 [142- | | | | 367] |
| | | | 161] | | | | |
| 18 | 4065 | FREASVCMQLLFGIDVKEVD | MAGE- | 20 | 5388 | KTRSGKVFQNKMANGNQPVK | CT46 [333- |
| | | | A11 [264- | | | | 352] |
| | | | 283 | | | | |
| 18 | 4066 | LETSFQVALSRKMAELVHFL | MAGE- | 20 | 5389 | IEEKSKLLEKFSLVQKEYEG | CTAGE5 [86- |
| | | | A12 [101- | | | | 105] |
| | | | 120] | | | | |
| 18 | 4067 | VIFSKASEYLQLVFGIEVVE | MAGE- | 20 | 5390 | GSSRGGGSGSGASDLGAGSK | DMRT1 [49- |
| | | | A2 [149-168] | | | | 68] |
| 18 | 4068 | MPKAGLLIIVLAIIAREGDC | MAGE- | 20 | 5391 | NSYNVRRTEGFSVTLDDLAP | EPHA2 [480- |
| | | | A3 [196-215] | | | | 499] |
| 18 | 4069 | GHSYILVTCLGLSYDGLLGD | MAGE- | 20 | 5392 | RPSRVHASEVESAVWYVKKF | FBXO39 [66- |
| | | | A8 [176-195] | | | | 85] |
| 18 | 4070 | SMPKAALLIIVLGVILTKDN | MAGE- | 20 | 5393 | QLAEIDIKLQELSAASPTIS | FSIP1 [319- |
| | | | A9 [194-213] | | | | 338] |
| 18 | 4071 | DPPRYQFLWGPRAYAETTKM | MAGE- | 20 | 5394 | GSHHTASPWNLSPFSKTSIH | GATA-3 [104- |
| | | | B1 [264-283] | | | | 123] |
| 18 | 4072 | WEVLNAVGVYAGREHFVYGE | MAGE- | 20 | 5395 | FVNLKNITYLVLDEADKMLD | HAGE [384- |
| | | | C2 [250-269] | | | | 403] |
| 18 | 4073 | AMGIPFIIQCDLCLKWRVLP | MORC1 [477- | 20 | 5396 | GSVAGAVKLNRQQTDMAVNW | HDAC2 [117- |
| | | | 496] | | | | 136] |
| 18 | 4074 | TLVHNGTSARATTTPASKST | MUC-1 [971- | 20 | 5397 | TESVSHFSDLGQSFSFHSGN | IGFS11 [332- |
| | | | 990] | | | | 351] |
| 18 | 4075 | QEANFHIIDNVELVVSAYCL | NLRP4 [574- | 20 | 5398 | TVEYELKYRNIGSETWKTII | IL13RA2 [66- |
| | | | 593] | | | | 85] |
| 18 | 4076 | LKPWLLEVNYSPALTLDCST | NYC- | 20 | 5399 | HSVPKRQRIRKLQIRNIPPH | KOC1 [72-91] |
| | | | TSPG [380- | | | | |
| | | | 399] | | | | |
| 18 | 4077 | TPGPAAYRQTDVRVTKFKAP | ODF3 [179- | 20 | 5400 | FHPGGLMKLRSREADLYRFI | KU-CT- |
| | | | 198] | | | | 1 [853-872] |
| 18 | 4078 | SELSLVAFILLLVVAFSKKW | ODF4 [85- | 20 | 5401 | RAHAETSKMKVLEYIANANG | MAGE-A11 [389-408] |
| | | | 104] | | | | |
| 18 | 4079 | GPQLPSWLQPERCAVFQCAQ | OIP5 [63-82] | 20 | 5402 | EEGPSTFPDLESEFQAALSR | MAGE-A6 [92- |
| | | | | | | | 111] |
| 18 | 4080 | HHPNIVGYRAFTEANDGSLC | PBK [93-112] | 20 | 5403 | QIKEPVTKAEMLESVIKNYK | MAGE- |
| | | | | | | | A8 [128-147] |
| 18 | 4081 | PNVECKSMRFGMLKDHQAVT | PIWIL2 [249- | 20 | 5404 | KEPVTKAEMLESVIKNYKRY | MAGE- |
| | | | 268] | | | | A9 [126-145] |
| 18 | 4082 | LQLWDLKFDTNFLSVPGRVL | PIWIL3 [453- | 20 | 5405 | DGEEHSVFGEPWKLITKDLV | MAGE- |
| | | | 472] | | | | B2 [234-253] |
| 18 | 4083 | MLLFSVLLLLSLVTGTQLGP | PRSS55 [1- | 20 | 5406 | STSTERSLKDSLTRKTKMLV | MAGE-B4 [98- |
| | | | 20] | | | | 117] |
| 18 | 4084 | GDFGSCRSVYSKQPYTEYIS | RAGE-1 [144- | 20 | 5407 | QATIDTADDATVMASESLSV | MAGE- |
| | | | 163] | | | | C2 [345-364] |
| 18 | 4085 | VYDYNCHVELIRLLRLEGEL | SART3 [109- | 20 | 5408 | LDVQIQHVVEGKRALSELTQ | MPHOSPH1 [1135- |
| | | | 128] | | | | 1154] |
| 18 | 4086 | MAAAPPSYCFVAFPPRAKDG | SCRN1 [1-20] | 20 | 5409 | ESLDQKLFQLQSKNMWLQQQ | NY-BR- |
| | | | 1 [1261- | | | | |
| | | | 1280] | | | | |
| 18 | 4087 | SCQDLQREISILQEQISHLQ | se57-1 [228- | 20 | 5410 | HKAEVEAIMEQLKELKQKGD | ODF2 [377- |
| | | | 247] | | | | 396] |
| 18 | 4088 | NPVLICLALSKATEYLVIIG | SLCO6A1 [382- | 20 | 5411 | TKYVFDSAPSHSISARTKAF | ODF3 [112- |
| | | | 401] | | | | 131] |
| 18 | 4089 | RRQEMRQFFTVGQQPQIPIT | SOX-6 [717- | 20 | 5412 | SVHLAWDLSRSLGAVVFSRV | OIP5 [90-109] |
| | | | 736] | | | | |
| 18 | 4090 | VDYKTVLQIDCGLQLANDSV | SPAG1 [542- | 20 | 5413 | NDLIEERYKASQDPFPAAII | PBK [124-143] |
| | | | 561] | | | | |
| 18 | 4091 | LKDSILSIVHVKGIVLVALA | SPAG9 [999- | 20 | 5414 | RGTTEIGMIGSKPFSTVKYK | PEPP2 [779- |
| | | | 1018] | | | | 798] |
| 18 | 4092 | HILSTSKGLIAGNLRYIEED | SPO11 [169- | 20 | 5415 | KRVNTRFFAQSGGRLQNPLP | PIWIL1 [742- |
| | | | 188] | | | | 761] |
| 18 | 4093 | VLWSKGTEILVNSSRVTVTP | TAG-1 [450- | 20 | 5416 | KTPKDSFTMSDGKEITFLEY | PIWIL2 [436- |
| | | | 469] | | | | 455] |
| 18 | 4094 | LPTLRSALFSRYSPHDWDQS | TEKT5 [78- | 20 | 5417 | PSVSQLSVLSLSGVMLTDVS | PRAME [346- |
| | | | 97] | | | | 365] |
| 18 | 4095 | HCPTCVALGTCFSAPSLPCP | TEX101 [139- | 20 | 5418 | YSEELFPEELSVVLGTNDLT | PRSS55 [111- |
| | | | 158] | | | | 130] |
| 18 | 4096 | TEALIVCEQDVSRMRRQLDE | TSGA10 [316- | 20 | 5419 | KFSGYPLYHSVYETYELVEK | PSMA [545- |
| | | | 335] | | | | 564] |
| 18 | 4097 | MRPEGSLTYRVPERLRQGFC | TSPY1 [1-20] | 20 | 5420 | RLNPHPNILMLHEVVFDRKS | RAGE-1 [56- |
| | | | | | | | 75] |
| 18 | 4098 | PPPPSQASSGQARMFPNAPY | WT1 [114- | 20 | 5421 | ELTKVRMARQKMSEIFPLTE | SART3 [127- |
| | | | 133] | | | | 146] |
| 19 | 4099 | VPTELPAYVRNLFLTGNQLA | 5T4 [85-104] | 20 | 5422 | ELEDIKVSLQRSVSTQKALE | SCP-1 [323- |
| | | | | | | | 342] |
| 19 | 4100 | IYDENSYWRNQNPGSLLQLP | ACRBP [301- | 20 | 5423 | TGEGEFNFSEVFSPVEDHLD | SCRN1 [215- |
| | | | 320] | | | | 234] |
| 19 | 4101 | IPRATIIYANISGHLCIAVE | ADAM2 [557- | 20 | 5424 | SILQEQISHLQFVIHSQHQN | se57-1 [237- |
| | | | 576] | | | | 256] |
| 19 | 4102 | NCSYGDFWEYTVERTKCLLE | ADAM29 [368- | 20 | 5425 | QQQEQIARQQQQLLQQQHKI | SOX-6 [232- |
| | | | 387] | | | | 251] |
| 19 | 4103 | FNQWKQNATDIMEAMLKRLV | AKAP-4 [412- | 20 | 5426 | IIEAKMELEEVTRLLNLKDK | SPAG1 [724- |
| | | | 431] | | | | 743] |
| 19 | 4104 | HLETVILGLLKTPAQYDASE | ANXA2 [94- | 20 | 5427 | SSSLSIKWDPVVPFRNESAV | TAG-1 [925- |
| | | | 113] | | | | 944] |
| 19 | 4105 | EKMENQEYKDAYKFAADVRL | BRET [324- | 20 | 5428 | QAIIQGFSYDLLKKIDSPQR | TEX14 [152- |
| | | | 343] | | | | 171] |
| 19 | 4106 | YHENKKLIRCKVEGFTHLYT | CALR3 [154- | 20 | 5429 | HVSDHNRLLHLARPKAQSDK | THEG [293- |
| | | | 173] | | | | 312] |
| 19 | 4107 | MVAVHQQQLLSWEEDRQKVL | CCDC62 [47- | 20 | 5430 | TVEKEMKSLARKAMDTESEL | TSGA10 [174- |
| | | | 66] | | | | 193] |
| 19 | 4108 | EKMKETVQKLKNARQEVVEK | CDCA1 [256- | 20 | 5431 | SAKEGTAFRMEAVQEGAAGV | TSPY1 [33- |
| | | | 275] | | | | 52] |
| 19 | 4109 | RGPLFPVDARGPFLRRGPPF | CTAGE5 [700- | | | | |
| | | | 719] | | | | |

### Example 19—Process for Personalized Vaccination

Process for personalized vaccination consists of 3 main steps as shown in FIG. 17. First, during the visit at the oncologist the patient provides saliva sample and tumor sample (biopsy) for tumor pathology. Second step is the matching of vaccine peptides with the patient's unique genetic code: Based on the determined HLA genotype of the patient and determined tumor type of the patient, 12 tumor and patient specific peptides are selected for the patient from the Hotspot Sequences listed in Table-25. Then, the vaccine will be prepared and after fill&finish plus QC release, and vaccine vials shipped to the clinical site. At the clinic, as third step of the process, vaccine is administered to the patient by the oncologist. The manufacturing of the personalized vaccine is carried out under GMP conditions. Vaccine selection and preparation can be performed during 6-8 weeks.

Eligibility criteria for personalized vaccination are:
≥12 immunogenic peptides (12 PEPIs) from
≥12 different cancer-specific antigens (expression rate (ER) in the disease ≥10%) and
AGP≥3 (expected number of antigens expressed in the patient's tumor)
On stock in the PEPI PANEL “Warehouse”

### Example 20—Feasibility Study for a “Simulated” Breast Cancer Clinical Trial

We describe here a model, where the peptide set (“warehouse”) consists of 100 immunogenic peptides derived from breast cancer specific TSAs (FIG. 18). These 100 peptides represent the peptide set for vaccine selection in this example. In this stock, there are available 100 mg of each peptide, that represents 25 peptide dose (for vaccination).

During the feasibility study, we screened 509 HLA-genotyped breast cancer subjects and identified 82 patients (16%) who were not eligible because less, than 12 peptides were found for those patients. This means, that 82% of patients could be treated with “patient-specific” vaccine from a panel consisting of 100 peptides. However, the amount (100 mg each) will only be enough for 32% of the patients if we intend to administer 3 doses/patient during the treatment, therefore 267 (52%), who will have not sufficient peptides in warehouse must be also excluded from this feasibility study. Consequently, 160 patients can be enrolled and treated by the administration of 3 consecutive vaccine doses. Manufacturing (GMP) can be performed within cca. 6 months.

### Example 21—Vaccine Selection for a Breast Cancer Patient (Patient-C of Example 22) and for a Colorectal Cancer Patient (Patient-D of Example 22)

The vaccine selection process from peptides listed in Table 25 is demonstrated here by two examples.

Patient-C's PIT vaccine described in Example 22 were designed during a completely personalized design process and manufactured individually, and demonstrated very high immunogenicity: 11 out of 12 (92%) vaccine peptides induced CD8+ T cell responses and 11/12 (92%) were resulted in CD4+ T cell specific immune responses.

By taking the HLA genotype and tumor pathology report (breast cancer) of Patient-C, patient matching process according to Example 16 resulted in 116 of 3286 sequences, selected from 38 breast cancer specific TSAs (according to selection criteria of, Expression rate (ER)≥10%). These 116 peptides are usable for vaccine selection for Patient-C and contain the PIT vaccine sequences. Three examples for random selection of 12 peptides from the 116 peptides are shown in Table 25 with expected AGP numbers.

PIT vaccine of Patient-C has an expected AGP value of 6.45, meaning that at least 6 vaccine-specific TSAs are likely expressed in the patient's tumor and are targeted by immune responses with at least 50% of probability. (AGP95 is 4, meaning that PIT vaccine peptides from at least 4 different TSAs TSAs are likely expressed in the patient's tumor and are targeted by immune responses in Patient-C with at least 95% probability.)

TABLE 29
Vaccine selection for Patient-C from the PEPI PANEL. Original vaccine means the
PIT vaccine designed during in a completely personalized design process for Patient-C (Example
22). The peptide set matched with Patient-C breast cancer indication resulted in 116 of 3286
Sequences selected from 38 of 58 Breast cancer specific TSAs). These 116 peptides are usable
for vaccine selection for Patient-C. Three examples for random selection of 12 peptides from the
116 peptides are shown in this table with calculated expected AGP numbers.

| | | Calculated | |
|---|---|---|---|
| Vaccines | SEQ ID NOS (AG[ER %] from Table 25) | expected AGP | CI95% |
| PIT vaccine | 5449 (SPAG9[88%]), 5432 (AKAP-4[85%]), 5433 (CTCFL[74%]), | 6.45 | [3, 9] |
| of Patient- | 5440 (MAGE-A11[59%]), 41 (FMR1NB[55%]), 5437 | | |
| C | (FSIP1[42%]), 5444 (NY-BR-1[47%]), 5439 (LDHC[35%]), 5438 | | |
| | (GATA-3[31%]), 440 (Survivin[68%]), 5443 (MAGE-C1[16%]), | | |
| | 111 (PRAME[49%]) | | |
| Random1 | 2 (ACRBP[40%]), 7 (AKAP-3[40%]), 18 (CDCA1[64%]), 40 | 4.91 | [2, 8] |
| | (FBXO39[38%]), 41 (FMR1NB[55%]), 42 (FSIP1[42%]), 57 | | |
| | (JARID1B[76%]), 60 (Lage-1[12%]), 66 (MAGE-A11[59%]), 68 | | |
| | (MAGE-A2[12%]), 74 (MAGE-A9[44%]), 80 (MAGE-C1[16%]) | | |
| Random2 | 107 (PIWIL2[94%]), 167 (AKAP-4[85%]), 183 (CTCFL[74%]), 192 | 7.96 | [5, 11] |
| | (EpCAM[76%]), 215 (JARID1B[76%]), 405 (ODF4[63%]), 440 | | |
| | (Survivin[68%]), 663 (MAGE-A11[59%]), 1539 (MAGE-A9[44%]), | | |
| | 857 (TDRD1[38%]), 1326 (PEPP2[60%]), 1441 (ODF4[63%]) | | |
| Random3 | 18 (CDCA1[64%]), 107 (PIWIL2[94%]), 215 (JARID1B[76%]), | 5.92 | [3, 9] |
| | 297 (TDRD1[38%]), 391 (MAGE-C1[16%]), 470 (AKAP-4[85%]), 490 | | |
| | (DKKL1[20%]), 498 (FBXO39[38%]), 1088 (PRAME[49%]), 1100 | | |
| | (SP17[47%]), 1800 (TSGA10[70%]) | | |

For Patient-D, a similar analysis was performed. During the patient matching process according to Example 19, 136 of 3286 sequences were selected from Table 25. (derived from 37 of 53 CRC specific TSAs), based on the HLA genotype and tumor pathology report (colorectal cancer, CRC) data of Patient-D. These 136 peptides are usable for vaccine selection for Patient-D. Three examples for random selection of 13 peptides from the 136 peptides are shown in Table 30, with calculated AGP numbers. Patient-D had a PIT vaccine consisting of 13 peptides, therefore the random selection was also performed to result in 13 peptide sets.

Patient-D's PIT vaccine described in Example 22 were designed during a completely personalized design process and manufactured individually, and demonstrated very high immunogenicity: 13 out of 13 (100%) vaccine peptides induced CD8+ T cell responses and 7/13 (54%) were resulted in CD4+ T cell specific immune responses.

PIT vaccine of Patient-D has an expected AGP value of 6.60, meaning that at least 6 vaccine-specific TSAs are likely expressed in the patient's tumor and are targeted by immune responses with at least 50% of probability. (AGP95 is 4, meaning that PIT vaccine peptides from at least 4 different TSAs are likely expressed in the patient's tumor and are targeted by immune responses in Patient-D with at least 95% probability.)

TABLE 30
Vaccine selection for Patient-D from the PEPI PANEL. Original vaccine means the
PIT vaccine designed during in a completely personalized design process for Patient-D (Example
22). The peptide set matched with Patient-D CRC indication resulted in 136 of 3286 Sequences
selected from 37 of 53 CRC specific TSAs). These 136 peptides are usable for vaccine selection
for Patient-D and contain the PIT vaccine sequences. Three examples for random selection of 13
peptides from the 116 peptides are shown in this table with calculated AGP numbers.

| | | Calculated | |
|---|---|---|---|
| | | expected | |
| Vaccines | SEQ ID NOS (AG[ER %] from Table 25) | AGP | CI95% |
| PIT vaccine | 5450 (TSP50[89%]), 5436 (EpCAM[88%]), 5434 | 6.60 | [4, 10] |
| of Patient-D | (CAGE1[74%]), 5447 (PIWIL1[48%]), 5448 (SPAG9[71%]), | | |
| | 2168 (AKAP-4[74%]), 5442 (MAGE-A8[44%]), 5435 | | |
| | (DPPA2[44%]), 40 (FBXO39[39%]), 5445 (NYD-TSPG[30%]), | | |
| | 5446 (PAGE4[33%]), 647 (HAGE[15%]), 1172 (Lage-1[13%]) | | |
| Random1 | 315 (5T4[85%]), 347 (DPPA2[44%]), 353 (FBXO39[39%]), 371 | 5.30 | [2, 8] |
| | (Lage-1[13%]), 383 (MAGE-A6[22%]), 384 (MAGE-A8[44%]), | | |
| | 412 (PIWIL2[80%]), 470 (AKAP-4[74%]), 486 (CXorf48[17%]), | | |
| | 639 (FBXO39[39%]), 647 (HAGE[15%]), 652 (IGFS11[55%]), | | |
| | 657 (Lage-1[13%]) | | |
| Random2 | 1157 (FBXO39[39%]), 1160 (GASZ[22%]), 1172 (Lage- | 6.91 | [4, 10] |
| | 1[13%]), 1183 (MAGE-A6[22%]), 1211 (PIWIL3[96%]), 1249 | | |
| | (5T4[85%]), 1256 (AKAP-4[74%]), 1262 (CCDC62[13%]), 1270 | | |
| | (DBPC[90%]), 1274 (EpCAM[88%]), 1300 (MAGE-A3[22%]), | | |
| | 1365 (ZNF165[43%]), 1372 (AKAP-3[83%]) | | |
| Random3 | 470 (AKAP-4[74%]), 486 (CXorf48[17%]), 639 (FBXO39[39%]), | 5.32 | [3, 8] |
| | 647 (HAGE[15%]), 652 (IGFS11[55%]), 657 (Lage-1[13%]), 658 | | |
| | (LDHC[15%]), 686 (NYD-TSPG[30%]), 696 (PIWIL3[96%]), 713 | | |
| | (SPAG1[55%]), 796 (MAGE-A1[14%]), 830 (PIWIL3[96%]), 929 | | |
| | (MAGE-A2[18%]) | | |

### Example 22—Personalised Immunotherapy (PIT) Design and Treatment for Ovarian-, Breast- and Colorectal Cancer

This Example provides proof of concept data from 4 metastatic cancer patients treated with personalized immunotherapy vaccine compositions to support the principals of binding of epitopes by multiple HLAs of a subject to induce cytotoxic T cell responses, on which the present disclosure is partly based on.

Composition for Treatment of Ovarian Cancer with P0001-PIT (Patient-A)

This example describes the treatment of an ovarian cancer patient with a personalised immunotherapy composition, wherein the composition was specifically designed for the patient based on her HLA genotype based on the disclosure described herein.

The HLA class I and class II genotype of a metastatic ovarian adenocarcinoma cancer patient (Patient-A) was determined from a saliva sample.

To make a personalized pharmaceutical composition for Patient-A thirteen peptides were selected, each of which met the following two criteria: (i) derived from an antigen that is expressed in ovarian cancers, as reported in peer reviewed scientific publications; and (ii) comprises a fragment that is a T cell epitope capable of binding to at least three HLA class I of Patient-A (Table 31). In addition, each peptide is optimized to bind the maximum number of HLA class II of the patient.

TABLE 31
Personalized vaccine of
ovarian cancer Patient-A.

| POC01 | | | | | | |
|---|---|---|---|---|---|---|
| vaccine | Tar- | Anti- | | | MAX | MAX |
| for | get | gen | 20mer | SEQ | HLA | HLA |
| Patient- | Anti- | Expres- | pep- | ID | class | class |
| A | gen | sion | tides | NO: | I | II |
| POC01_ | AKAP4 | 89% | NSLQ | 5933 | 3 | 5 |
| P1 | | | KQLQ | | | |
| | | | AVLQ | | | |
| | | | WIAA | | | |
| | | | SQFN | | | |
| POC01_ | BORIS | 82% | SGDE | 5934 | 4 | 2 |
| P2 | | | RSDE | | | |
| | | | IVLT | | | |
| | | | VSNS | | | |
| | | | NVEE | | | |
| POC01_ | SPAG9 | 76% | VQKE | 5935 | 3 | 3 |
| P3 | | | DGRV | | | |
| | | | QAFG | | | |
| | | | WSLP | | | |
| | | | QKYK | | | |
| POC01_ | OY- | 75% | EVES | 5936 | 3 | 4 |
| P4 | TES-1 | | TPMI | | | |
| | | | MENI | | | |
| | | | QELI | | | |
| | | | RSAQ | | | |
| POC01_ | 5P17 | 69% | AYFE | 5937 | 3 | 1 |
| P5 | | | SLLE | | | |
| | | | KREK | | | |
| | | | TNFD | | | |
| | | | PAEW | | | |
| POC01_ | WT1 | 63% | PSQA | 5938 | 4 | 1 |
| P6 | | | SSGQ | | | |
| | | | ARMF | | | |
| | | | PNAP | | | |
| | | | YLPS | | | |
| POC01_ | HIWI | 63% | RRSI | 5939 | 3 | 4 |
| P7 | | | AGFV | | | |
| | | | ASIN | | | |
| | | | EGMT | | | |
| | | | RWFS | | | |
| POC01_ | PRAME | 60% | MQDI | 5940 | 3 | 4 |
| P8 | | | KMIL | | | |
| | | | KMVQ | | | |
| | | | LDSI | | | |
| | | | EDLE | | | |
| POC01_ | AKAP- | 58% | ANSV | 5941 | 3 | 4 |
| P9 | 3 | | VSDM | | | |
| | | | MVSI | | | |
| | | | MKTL | | | |
| | | | KIQV | | | |
| POC01_ | MAGE- | 37% | REAL | 5942 | 3 | 2 |
| P10 | A4 | | SNKV | | | |
| | | | DELA | | | |
| | | | HFLL | | | |
| | | | RKYR | | | |
| POC01_ | MAGE- | 37% | ETSY | 5943 | 3 | 4 |
| P11 | A9 | | EKVI | | | |
| | | | NYLV | | | |
| | | | MLNA | | | |
| | | | REPI | | | |
| POC01_ | MAGE- | 52% | DVKE | 5944 | 3 | 4 |
| P12a | A10 | | VDPT | | | |
| | | | GHSF | | | |
| | | | VLVT | | | |
| | | | SLGL | | | |
| POC01_ | BAGE | 30% | SAQL | 5945 | 3 | 2 |
| P12b | | | LQAR | | | |
| | | | LMKE | | | |
| | | | ESPV | | | |
| | | | VSWR | | | |

Eleven PEPI3 peptides in this immunotherapy composition can induce T cell responses in Patient-A with 84% probability and the two PEPI4 peptides (P0001-P2 and P0001-P5) with 98% probability, according to the validation of the PEPI test shown in Table 7. T cell responses target 13 antigens expressed in ovarian cancers. Expression of these cancer antigens in Patient-A was not tested. Instead the probability of successful killing of cancer cells was determined based on the probability of antigen expression in the patient's cancer cells and the positive predictive value of the ≥1 PEPI3+ test (AGP count). AGP count predicts the effectiveness of a vaccine in a subject: Number of vaccine antigens expressed in the patient's tumor (ovarian adenocarcinoma) with PEPI. The AGP count indicates the number of tumor antigens that the vaccine recognizes and induces a T cell response against the patient's tumor (hit the target). The AGP count depends on the vaccine-antigen expression rate in the subject's tumor and the HLA genotype of the subject. The correct value is between 0 (no PEPI presented by any expressed antigen) and maximum number of antigens (all antigens are expressed and present a PEPI).

The probability that Patient-A will express one or more of the 13 antigens is shown in FIGS. 19A-B. AGP95 (AGP with 95% probability)=5, AGP50 (the mean—expected value—of the discrete probability distribution)=7.9, mAGP (probability that AGP is at least 2)=100%, AP=13.

A pharmaceutical composition for Patient-A may be comprised of at least 2 from the 13 peptides (Table 31), because the presence in a vaccine or immunotherapy composition of at least two polypeptide fragments (epitopes) that can bind to at least three HLAs of an individual (≥2 PEPI3+) was determined to be predictive for a clinical response. The peptides are synthetized, dissolved in a pharmaceutically acceptable solvent and mixed with an adjuvant prior to injection. It is desirable for the patient to receive personalized immunotherapy with at least two peptide vaccines, but preferable more to increase the probability of killing cancer cells and decrease the chance of relapse.

For treatment of Patient-A, the 13 peptides were formulated as 4×3 or 4 peptide (P0001/1, P0001/2, P0001/3, P0001/4). One treatment cycle is defined as administration of all 13 peptides within 30 days.

### Patient History:

Diagnosis: Metastatic ovarian adenocarcinoma

### Age: 51

Family anamnesis: colon and ovary cancer (mother) breast cancer (grandmother)

### Tumor Pathology:

2011: first diagnosis of ovarian adenocarcinoma; Wertheim operation and chemotherapy; lymph node removal
2015: metastasis in pericardial adipose tissue, excised
2016: hepatic metastases
2017: retroperitoneal and mesenteric lymph nodes have progressed; incipient peritoneal carcinosis with small accompanying ascites

### Prior Therapy:

### 2012: Paclitaxel-carboplatin (6×)

### 2014: Caelyx-carboplatin (1×)

2016-2017 (9 months): Lymparza (Olaparib) 2×400 mg/day, oral
2017: Hycamtin inf. 5×2.5 mg (3× one seria/month)

PIT vaccine treatment began on 21 Apr. 2017. FIG. 20.

2017-2018: Patient-A received 8 cycles of vaccination as add-on therapy, and lived 17 months (528 days) after start of the treatment. During this interval, after the 3^{rd} and 4^{th} vaccine treatment she experienced partial response as best response. She died in October 2018.

An interferon (IFN)-γ ELISPOT bioassay confirmed the predicted T cell responses of Patient-A to the 13 peptides. Positive T cell responses (defined as >5 fold above control, or >3 fold above control and >50 spots) were detected for all 13 20-mer peptides and all 13 9-mer peptides having the sequence of the PEPI of each peptide capable of binding to the maximum HLA class I alleles of Patient-A (FIG. 21).

Patient' tumor MRI findings (Baseline Apr. 15, 2016) (BL: baseline for tumor response evaluation on FIG. 22)
Disease was confined primarily to liver and lymph nodes. The use of MRI limits detection of lung (pulmonary) metastasis
May 2016-January 2017: Olaparib treatment (FU1: follow up 1 on FIG. 22)
Dec. 25, 2016 (before PIT vaccine treatment) There was dramatic reduction in tumor burden with confirmation of response obtained at (FU2: follow up 2 on FIG. 22)
January-March 2017—TOPO protocol (topoisomerase)
Apr. 6, 2017 (FU3 on FIG. 22) demonstrated regrowth of existing lesions and appearance of new lesions leading to disease progression. Peritoneal carcinomatosis with increased amount of ascites. Progressive hepatic tumor and lymph node

### Apr. 21, 2017 START PIT

Jul. 26, 2017 (after the 2^{nd} Cycle of PIT): (FU4 on FIG. 22) Progression/Pseudo-Progression
Rapid progression in lymph nodes, hepatic, retroperitoneal and thoracic areas, significant pleural fluid and ascites. Initiate Carboplatin, Gemcitabine, Avastin.
   Sep. 20, 2017 (after 3 Cycles of PIT): (FU5 on FIG. 22) Partial Response
Complete remission in the pleural region/fluid and ascites
Remission in hepatic, retroperitoneal area and lymph nodes
The findings suggest pseudo progression.
   Nov. 28, 2017 (after 4 Cycles of PIT): (FU6 on FIG. 22) Partial Response
Complete remission in the thoracic region. Remission in hepatic, retroperitoneal area and lymph nodes

### Apr. 13, 2018: Progression

Complete remission in the thoracic and retroperitoneal regions. Progression in hepatic centers and lymph nodes
   Jun. 12, 2018: Stable disease
Complete remission in the thoracic and retroperitoneal regions. Minimal regression in hepatic centers and lymph nodes

### July 2018: Progression

October 2018: Patient-A died
Partial MRI data for Patient-A is shown in Table 32 and FIG. 22.

TABLE 32
Summary Table of Lesions Responses

| | Baseline | FU1 | FU2 | FU3 | FU4 | FU5 | FU6 | | |
|---|---|---|---|---|---|---|---|---|---|
| Lesion/ | (% Δ | (% Δ | (% Δ | (% Δ | (% Δ | (% Δ | (% Δ | Best | PD |
| Time | from | from | from | from | from | from | from | Response | Time |
| Point | BL) | BL) | BL) | BL) | BL) | BL) | BL) | Cycle | Point |
| TL1 | NA | −56.1 | −44.4 | −44.8 | +109.3 | −47.8 | −67.3 | FU6 | FU4 |
| TL2 | NA | −100.0 | −100.0 | −47.1 | −13.1 | −100.0 | −100.0 | FU1 | FU3 |
| TL3 | NA | −59.4 | −62.3 | −62.0 | −30.9 | −66.7 | −75.9 | FU6 | FU4 |
| TL4 | NA | −65.8 | −100.0 | −100.0 | −100.0 | −100.0 | −100.0 | FU2 | NA |
| SUM | NA | −66.3 | −76.0 | −68.9 | −23.5 | −78.2 | −85.2 | FU6 | FU4 |

### Design, Safety and Immunogenicity of Personalised Immunotherapy Composition PBRC01 for Treatment of Metastatic Breast Cancer (Patient-B)

The HLA class I and class II genotype of metastatic breast cancer Patient-B was determined from a saliva sample. To make a personalized pharmaceutical composition for Patient-B twelve peptides were selected, each of which met the following two criteria: (i) derived from an antigen that is expressed in breast cancers, as reported in peer reviewed scientific publications; and (ii) comprises a fragment that is a T cell epitope capable of binding to at least three HLA class I of Patient-B (Table 33). In addition, each peptide is optimized to bind the maximum number of HLA class II of the patient. The twelve peptides target twelve breast cancer antigens. The probability that Patient-B will express one or more of the 12 antigens is shown in FIGS. 23A-C.

TABLE 33
12 peptides for Patient-B breast
cancer patient

| BRC01 | Tar- | Anti- | | | MAXHL | |
|---|---|---|---|---|---|---|
| vaccine | get | gen | 20mer | SEQ | A | MAXHLA |
| pep- | Anti- | Expres- | pep- | ID | Class | Class |
| tides | gen | sion | tide | NO: | I | II |
| PBRC01_ | FSIP1 | 49% | ISDT | 5946 | 3 | 6 |
| cP1 | | | KDYF | | | |
| | | | MSKT | | | |
| | | | LGIG | | | |
| | | | RLKR | | | |
| PBRC01_ | SPAG9 | 88% | FDRN | 5947 | 3 | 2 |
| cP2 | | | TESL | | | |
| | | | FEEL | | | |
| | | | SSAG | | | |
| | | | SGLI | | | |
| PBRC01_ | AKAP4 | 85% | SQKM | 5948 | 3 | 6 |
| cP3 | | | DMSN | | | |
| | | | IVLM | | | |
| | | | LIQK | | | |
| | | | LLNE | | | |
| PBRC01_ | BORIS | 71% | SAVF | 5949 | 3 | 6 |
| cP4 | | | HERY | | | |
| | | | ALIQ | | | |
| | | | HQKT | | | |
| | | | HKNE | | | |
| PBRC01_ | MAGE- | 59% | DVKE | 5950 | 3 | 4 |
| cP5 | A11 | | VDPT | | | |
| | | | SHSY | | | |
| | | | VLVT | | | |
| | | | SLNL | | | |
| PBRC01_ | NY- | 49% | ENAH | 5951 | 3 | 2 |
| cP6 | SAR- | | GQSL | | | |
| | 35 | | EEDS | | | |
| | | | ALEA | | | |
| | | | LLNF | | | |
| PBRC01_ | HOM- | 47% | MASF | 5952 | 3 | 5 |
| cP7 | TES- | | RKLT | | | |
| | 85 | | LSEK | | | |
| | | | VPPN | | | |
| | | | HPSR | | | |
| PBRC01_ | NY- | 47% | KRAS | 5953 | 3 | 6 |
| cP8 | BR-1 | | QYSG | | | |
| | | | QLKV | | | |
| | | | LIAE | | | |
| | | | NTML | | | |
| PBRC01_ | MAGE- | 44% | VDPA | 5954 | 3 | 8 |
| cP9 | A9 | | QLEF | | | |
| | | | MFQE | | | |
| | | | ALKL | | | |
| | | | KVAE | | | |
| PBRC01_ | SCP- | 38% | EYER | 5955 | 3 | 3 |
| cP10 | 1 | | EETR | | | |
| | | | QVYM | | | |
| | | | DLNN | | | |
| | | | NIEK | | | |
| PBRC01_ | MAGE- | 37% | PEIF | 5956 | 3 | 3 |
| cP11 | A1 | | GKAS | | | |
| | | | ESLQ | | | |
| | | | LVFG | | | |
| | | | IDVK | | | |
| PBRC01_ | MAGE- | 21% | DSES | 5957 | 4 | 2 |
| cP12 | C2 | | SFTY | | | |
| | | | TLDE | | | |
| | | | KVAE | | | |
| | | | LVEF | | | |

Predicted efficacy: AGP95=4; 95% likelihood that the PIT Vaccine induces CTL responses against 4 TSAs expressed in the breast cancer cells of Patient-B. Additional efficacy parameters: AGP50=6.45, mAGP=100%, AP=12.

For treatment of Patient-B the 12 peptides were formulated as 4×3 peptide (PBR01/1, PBR01/2, PBR01/3, PBR01/4). One treatment cycle is defined as administration of all 12 different peptide vaccines within 30 days (FIG. 23C).

### Patient History:

2013: Diagnosis: breast carcinoma diagnosis; CT scan and bone scan ruled out metastatic disease.
2014: bilateral mastectomy, postoperative chemotherapy
2016: extensive metastatic disease with nodal involvement both above and below the diaphragm. Multiple liver and pulmonary metastases.

### Therapy:

### 2013-2014: Adriamycin-Cyclophosphamide and Paclitaxel

2017: Letrozole, Palbociclib and Gosorelin and PIT vaccine
2018: Worsening conditions, patient died in January

PIT vaccine treatment began on 7 Apr. 2017. treatment schedule of Patient-B and main characteristics of disease are shown in Table 34.

TABLE 34
Treatment and response of Patient-B

| | Date (2017) | | | | | |
|---|---|---|---|---|---|---|
| | Mar | May | Jun | Sep | Nov | Dec |
| | PIT Vaccine | | | | | |
| | Palbocyclib | | | | | |
| | Letrozole | | | | | |
| Treatment regimen | Gosorelin | | Anticancer drug treatment interruption | | | |
| Neutrophils | ND | 1.1 | 4.5 | 3.4 | 2.4 | 3 |
| (1.7-3.5/mm³) | | | | | | |
| CEA | 99 | 65 | 23 | 32 | 128 | 430 |
| (<5.0 ng/ml) | | | | | | |
| CA 15-3 | 322 | 333 | 138 | 76 | 272 | 230 |
| (<31.3 U/ml) | | | | | | |
| T1: Right axillar | 15 mm & | 9 mm & | nd* | nd | nd | 6 mm & 0 |
| lymph node | 11.6 | 2.0 | | | | SUVₘₐₓ |
| | SUVₘₐₓ | SUVₘₐₓ | | | | |
| T2: Right lung | 10 mm & | 7 mm & 0 | nd | nd | nd | 4 mm & 0 |
| metastasis | 4.8 | SUVₘₐₓ | | | | SUVₘₐₓ |
| | SUVₘₐₓ | | | | | |
| Left iliac bone | Non | Regression | nd | nd | nd | Regression & |
| metastasis | measurable | & 0 | | | | 0 SUVₘₐₓ |
| | & 4.0 | SUVₘₐₓ | | | | |
| | SUVₘₐₓ | | | | | |
| Multiple liver | Non | Partial | nd | nd | nd | Progression & |
| metastases | measurable | regression | | | | 16.8 SUVₘₐₓ |
| | & 11.5 | & 6.1 | | | | |
| | SUVₘₐₓ | SUVₘₐₓ | | | | |

*no data

It was predicted with 95% confidence that 8-12 vaccine peptides would induce T cell responses in Patient-B. Peptide-specific T cell responses were measured in all available PBMC samples using an interferon (IFN)-γ ELISPOT bioassay (FIG. 24). The results confirmed the prediction: Nine peptides reacted positive demonstrating that T cells can recognize Patient-B's tumor cells expressing FISP1, BORIS, MAGE-A11, HOM-TES-85, NY-BR-1, MAGE-A9, SCP1, MAGE-A1 and MAGE-C2 antigens. Some tumor specific T cells were present after the 1^{st} vaccination and boosted with additional treatments (e.g. MAGE-A1) others induced after boosting (e.g. MAGE-A9). Such broad tumor specific T cell responses are remarkable in a late stage cancer patient.

### Patient-B History and Results

### Mar. 7, 2017: Prior PIT Vaccine Treatment

Hepatic multi-metastatic disease with truly extrinsic compression of the origin of the choledochal duct and massive dilatation of the entire intrahepatic biliary tract. Celiac, hepatic hilar and retroperitoneal adenopathy
March 2017: Treatment initiation—Letrozole, Palbociclib, Gosorelin & PIT Vaccine
May 2017: Drug interruption
May 26 2017: After 1 cycle of PIT
83% reduction of tumor metabolic activity (PET CT) liver, lung lymphnodes and other metastases.
June 2017: Normalized Neutrophils values indicate Palbociclib interruption as affirmed by the patient

### 4 Months Delayed Rebound of Tumor Markers

March to May 2017: CEA and CA remained elevated consistently with the outcome of her anti-cancer treatment (Ban, Future Oncol 2018)
June to September 2017: CEA and CA decreased consistently with the delayed responses to immunotherapies

### Quality of Life

February to March 2017: Poor, hospitalized with jaundice

### April to October 2017: Excellent

November 2017: Worsening conditions (tumor escape?)
January 2018: Patient-B died.
Immunogenicity results are summarized in FIG. 24.

Clinical outcome measurements of the patient: One month prior to the initiation of PIT vaccine treatment PET CT documented extensive DFG avid disease with nodal involvement both above and below the diaphragm (Table 34). She had progressive multiple hepatic, multifocal osseous and pulmonary metastases and retroperitoneal adenopathy. Her intrahepatic enzymes were elevated consistent with the damage caused by her liver metastases with elevated bilirubin and jaundice. She accepted Letrozole, Palbociclib and Gosorelin as anti-cancer treatment. Two month after initiation of PIT vaccinations the patient felt very well and her quality of life normalized. In fact, her PET CT showed a significant morphometabolic regression in the liver, lung, bone and lymph node metastases. No metabolic adenopathy was identifiable at the supra-diaphragmatic stage.

The combination of Palblocyclib and the personalised vaccine was likely to have been responsible for the remarkable early response observed following administration of the vaccine. Palbocyclib has been shown to improve the activity of immunotherapies by increasing TSA presentation by HLAs and decreasing the proliferation of Tregs (Goel et al. Nature. 2017:471-475). The results of Patient-B treatment suggest that PIT vaccine may be used as add-on to the state-of-art therapy to obtain maximal efficacy.

Patient-B's tumor biomarkers were followed to disentangle the effects of state-of-art therapy from those of PIT vaccine. Tumor markers were unchanged during the initial 2-3 months of treatment then sharply dropped suggesting of a delayed effect, typical of immunotherapies (Table 34). Moreover, at the time the tumor biomarkers dropped the patient had already voluntarily interrupted treatment and confirmed by the increase in neutrophil counts.

After the 5^{th} PIT treatment the patient experienced symptoms. The levels of tumor markers and liver enzymes were increased again. 33 days after the last PIT vaccination, her PET CT showed significant metabolic progression in the liver, peritoneal, skeletal and left adrenal site confirming the laboratory findings. The discrete relapse in the distant metastases could be due to potential immune resistance; perhaps caused by downregulation of both HLA expression that impairs the recognition of the tumor by PIT induced T cells. However, the PET CT had detected complete regression of the metabolic activity of all axillary and mediastinal axillary supra-diaphragmatic targets (Table 34). These localized tumor responses may be accounted to the known delayed and durable responses to immunotherapy, as it is unlikely that after anti-cancer drug treatment interruption these tumor sites would not relapse.

Personalised Immunotherapy Composition for Treatment of a Patient with Metastatic Breast Carcinoma (Patient-C)

PIT vaccine similar in design to that described for Patient-A and Patient-B was prepared for the treatment of a patient (Patient-C) with metastatic breast carcinoma. PIT vaccine contained 12 PEPIs. The PIT vaccine has a predicted efficacy of AGP=4. The patient's treatment schedule is shown in FIG. 25.

### Tumor Pathology

2011 Original tumor: HER2−, ER+, sentinel lymph node negative
2017 Multiple bone metastases: ER+, cytokeratin 7+, cytokeratin 20−, CA125−, TTF1−, CDX2−

### Treatments

2011 Wide local resection, sentinel lymph nodes negative; radiotherapy
2017—Anti-cancer therapy (Tx): Letrozole (2.5 mg/day), Denosumab;

Radiation (Rx): one bone

PIT vaccine (3 cycles) as add-on to standard of care

Bioassay confirmed positive T cell responses (defined as >5 fold above control, or >3 fold above control and >50 spots) to 11 out of the 12 20-mer peptides of the PIT vaccine and 11 out of 12 9-mer peptides having the sequence of the PEPI of each peptide capable of binding to the maximum HLA class I alleles of the patient (FIGS. 26A-B). Long-lasting memory T-cell responses were detected after 14 months of the last vaccination (FIGS. 26C-D).

### Treatment Outcome

Clinical results of treatment of Patient-C are shown in Table 35. Patient-C has partial response and signs of healing bone metastases.

TABLE 35
Clinical results of treatment of breast cancer Patient-C

| | | | +150 days* | +388 days* |
|---|---|---|---|---|
| | Before PIT | +70 days* (10w) | (21w) | (55w) |
| Bone Biopsy | Met. breast | Not done | RIB5 is negative | Not done |
| | cancer DCIS | | | |
| PET CT | Multiple | Only RIBS is | Not done | Not done |
| | metastases | DFG avid | | |
| CT | Multiple | Not done | Not done | Healing bone |
| | metastases | | | mets (sclerotic |
| | | | | foci) |
| CA-15-3 | 87 | 50 | 32 | 24 |

*After 3rd cycle of PIT vaccination

Immune responses are shown on FIG. 26. Predicted Immunogenicity, PEPI=12 (CI95% [8, 12] Detected Immunogenicity: 11 (20-mers) & 11 (9-mers) antigen specific T cell responses following 3 PIT vaccinations (FIG. 26A, B). After 4.5, 11 or 14 months of the last vaccination, PIT vaccine-specific immune response could still be detected (FIG. 26 C, D).
Personalised Immunotherapy Composition for Treatment of Patient with Metastatic Colorectal Cancer (Patient-D)

### Tumor Pathology

2017 (February) mCRC (MSS) with liver metastases, surgery of primer tumor (in sigmoid colon). pT3 pN2b (8/16) M1. KRAS G12D, TP53-C135Y, KDR-Q472H, MET-T1010I mutations. SATB2 expression. EGFR wt, PIK3CA-I391M (non-driver).
2017 (June) Partial liver resection: KRAS-G12D (35G>A) NRAS wt,
2018 (May) 2^{nd} resection: SATB2 expression, lung metastases 3→21

### Treatments

2017 FOLFOX-4 (oxaliplatin, Ca-folinate, 5-FU)→allergic reaction during 2^{nd} treatment
   DeGramont (5-FU+Ca-folinate)
2018 (June)→FOLFIRI plus ramucirumab, biweekly; chemoembolization
2018 (October) PIT vaccination (13 patient-specific peptides, 4 doses) as add-on to standard of care.
   The patient's treatment schedule is shown in FIG. 27.

### Treatment Outcome

Patient in good overall condition, disease progression in lungs after 8 months confirmed by CT. Both PIT induced and pre-existing T cell responses were measured by enriched
Fluorospot from PBMC, using 9 mer and 20 mer peptides for stimulation (FIGS. 28A-B). Summary of immune response rate and immunogenicity results prove the proper design for target antigen selection as well as for the induction of multi-peptide targeting immune responses, both CD4+ and CD8+ specific ones.

TABLE 36
Summary table of immunological analysis of Patient A-D

| | Measured immunogenicity for the different | | | |
|---|---|---|---|---|
| | vaccine peptides* | | | |
| Patient ID | CD4+ T cells | | CD8+ T cells | |
| Patient-A | 13/13 | (100%) | 13/13 | (100%) |
| Patient-B | 9/12 | (75%) | 1/12 | (8%) |
| Patient-C | 11/12 | (92%) | 11/12 | (92%) |
| Patient-D | 7/13 | (54%) | 13/13 | (100%) |

| | | |
|---|---|---|
| IRR (ratio of immune responder patients) | 4/4 | 4/4 |
| Ratio of immunogenic peptides (median) | 10/12-13 | 10/12-13 |

*Following 1-3 cycles of vaccination

1. A method of providing immunotherapy to a subject in need thereof, the method comprising: administering to the individual a pharmaceutical composition, comprising i) two or more different peptides consisting of an amino acid sequence selected from the group consisting of SEQ ID Nos: 1 to 2786 and 5432 to 5931 and ii) a pharmaceutically acceptable adjuvant, diluent, carrier, preservative, or a combination thereof, thereby inducing an immune response.

2. The method of claim 1, wherein the pharmaceutical composition comprises at least 3, at least 4, at least 5, at least 6, at least 7, at least 8, at least 9, at least 10, at least 11, or at least 12 different peptides, wherein each peptide consists of an amino acid sequence selected from the group consisting of SEQ ID Nos: 1 to 2786 and 5432 to 5931.

3. The method of claim 1, wherein the adjuvant comprises an aluminium salt, saponin, Lipid A, or a water-in-oil emulsion.

4. The method according to claim 1, wherein the immunotherapy is a treatment for cancer.

5. The method of claim 6, wherein the cancer is bladder cancer, brain cancer, breast cancer, colorectal cancer, gastric cancer, hepatocellular cancer, leukemia, lung cancer, lymphoma, melanoma, ovarian cancer, pancreatic cancer, pediatric cancer, thyroid cancer, prostate cancer, kidney cancer, head and neck cancer, esophageal cancer and cervical cancer.

6. A pharmaceutical composition, comprising i) two or more different peptides consisting of an amino acid sequence selected from the group consisting of SEQ ID Nos: 1 to 2786 and 5432 to 5931 and ii) a pharmaceutically acceptable adjuvant, diluent, carrier, preservative, or a combination thereof.

7. The pharmaceutical composition of claim 8, comprising at least 3, at least 4, at least 5, at least 6, at least 7, at least 8, at least 9, at least 10, at least 11, or at least 12 different peptides, wherein each peptide comprises an amino acid sequence selected from the group consisting of SEQ ID Nos: 1 to 2786 and 5432 to 5931.

8. The pharmaceutical composition of claim 8, wherein the adjuvant comprises an aluminium salt, saponin, Lipid A, or a water-in-oil emulsion.
